(19) United States
(12) Patent Application Publication
(10) Pub. No.: US 2024/0216355 A1
(43) Pub. Date: Jul. 4, 2024

(54) PHARMACEUTICAL COMPOSITIONS FOR USE IN THE PREVENTION AND TREATMENT OF A DISEASE OR DISORDER CAUSED BY OR ASSOCIATED WITH ONE OR MORE PREMATURE TERMINATION CODONS

(71) Applicant: Monte Rosa Therapeutics, Inc., Boston, MA (US)

(72) Inventors: Bernhard Fasching, Basel (CH); Gerald Gavory, Basel (CH); Lev Osherovich, Basel (CH)

(21) Appl. No.: 18/471,563

(22) Filed: Sep. 21, 2023

Related U.S. Application Data
(63) Continuation of application No. PCT/IB2022/000150, filed on Mar. 22, 2022.

(30) Foreign Application Priority Data
Mar. 22, 2021 (CH) 00308/21

(51) Int. Cl. A61K 31/454; A61K 31/4545; A61K 31/496; A61K 31/4995; A61K 31/5377; A61K 31/5386; A61K 31/7048; A61P 11/00; A61P 35/00

(52) U.S. Cl. CPC A61K 31/454; A61K 31/4545; A61K 31/496; A61K 31/4995; A61K 31/5377; A61K 31/5386; A61K 31/7048; A61P 11/00; A61P 35/00

(57) ABSTRACT

The present disclosure relates to a compound of formula I or a pharmaceutically acceptable salt thereof

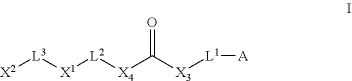

wherein A is

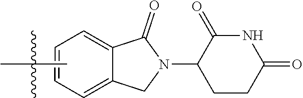

X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy; X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy; X³ is —NH—, —O—; X⁴ is —NH—, —CH₂—; L¹ is a covalent bond, C₁₋₆ alkyl, which is unsubstituted or substituted with one or more of C₁₋₄ alkyl, halogen; L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen, for use in the prevention and treatment of a disease or disorder caused by or associated with one or more premature termination codons in a monotherapy or in a combined therapy with an aminoglycoside or a pharmaceutically acceptable salt thereof.

### CROSS-REFERENCE

This application is a continuation of International Patent Application No. PCT/IB2022/000150, filed Mar. 22, 2022, which claims priority to Swiss Patent Application Number 00308/21, filed Mar. 22, 2021, the contents of each of which are incorporated herein by reference in its entirety.

### FIELD OF THE DISCLOSURE

The present disclosure relates to GSPT1 modulators, in particular a compound of formula I or a pharmaceutically acceptable salt thereof for use in the prevention and treatment of a disease or disorder caused by or associated with one or more premature termination codons in a monotherapy or a combined therapy with an aminoglycoside or a pharmaceutically acceptable salt thereof, as well as to pharmaceutical combinations comprising a compound of formula I or a pharmaceutically acceptable salt thereof and an aminoglycoside or a pharmaceutically acceptable salt thereof

### Background

Readthrough compounds have received much attention recently for their use in the treatment of genetic disorders that are caused by nonsense mutations. Nonsense mutations create premature termination codons resulting in either no formation of the target protein or formation of a truncated/defective protein. The ability to readthrough nonsense mutations allows to restore synthesis of the full-length protein, which includes maximally one amino acid difference to the wildtype protein and thus should be fully (or at least partially) active.

Thus, research has focused on compounds with readthrough activity of premature termination codons (PTCs) as a potential treatment strategy for nonsense mutation-mediated genetic disorders. For example, the aminoglycoside antibiotics class (e.g., gentamicin, paromomycin, G418 and its derivatives NB74 and NB84) has been shown to have the ability to induce readthrough of PTC (Keeling and Bedwell, 2005; Zingman et al., 2007). The therapeutic potential of aminoglycosides has been evaluated for many different genetic models, such as cystic fibrosis (see, e.g., Du et al., 2002, J. Mol. Med. 80:595-604; Howard et al., 1996; Bedwell et al., 1997), muscular dystrophy (see, e.g., Loufrani et al., 2004, Arterioscler. Thromb. Vasc. Biol. 24: 671-676; Howard et al., 2000; Loufrani et al., 2004), and others. In addition, clinical trials have also indicated that aminoglycosides can induce some functional protein production; however, the therapeutic benefits seem to be limited, for example because the systemic toxicity of most commercial aminoglycosides in mammals (Mingeot-Leclercq and Tulkens, 1999, Antimicrob. Agents Chemother. 43:1003-1012; Guan et al., 2000, Hum. Mol. Genet. 9:1787-1793).

Other compounds that have been investigated include e.g. the non-aminoglycosides Ataluren (for the treatment of Duchenne muscular dystrophie), RTC13 and RTC14, and macrolide antibiotics, such as tylosin and azithromycin) have been described as well.

Multiple GSPT1 degraders have been reported including CC-885 and CC-90009 (Matyskiela et al., 2016, Nature, 535, 252-257; Lu et al., 2019, Blood, 134, 405; Lopez-Girona et al., 2019, Blood, 134, 2703). These degraders exhibit strong tumoricidal activity against acute myeloid leukemia (AML) cells and CC-90009 has recently entered clinical trials in patients with relapsed or refractory AML (Uy et al., 2019, Blood, 134, 232; Jin et al., 2019, Blood, 134, 3940; Fan et al., 2019, Blood, 134, 2547).

However, there is still a great need for compounds with enhanced readthrough activity but reduced toxicity to restore synthesis of the full-length protein for use in the treatment of nonsense mutation mediated genetic diseases (Nudelman et al., 2006, Bioorg. Med. Chem. Lett. 16:6310-6315; Rebibo-Sabbah et al., 2007, Hum. Genet. 122:373-381).

### SUMMARY OF THE DISCLOSURE

The present disclosure relates to GSPT1 modulators for use in the prevention and treatment of a disease or disorder caused by or associated with one or more premature termination codons, in particular to a compound of formula I or pharmaceutically acceptable salts or stereoisomer thereof

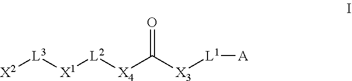

wherein A is

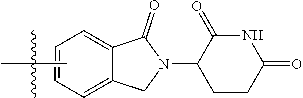

X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆ alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—; X⁴ is —NH—, —CH₂—;
L¹ is a covalent bond, C₁₋₆ alkyl, which is unsubstituted or substituted with one or more of C₁₋₄ alkyl, halogen; L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen,
for use in the prevention and treatment of a disease or disorder caused by or associated with one or more premature termination codons.

The compound or a pharmaceutically acceptable salt or stereoisomer may be used in monotherapy or in a combined therapy with an aminoglycoside or pharmaceutically acceptable salts thereof.

In some embodiments, the one or more premature termination codon is UGA or UAG or UAA.

In some embodiments, the compound of formula I or pharmaceutically acceptable salt or stereoisomer thereof for is a compound of formula IVa, IVb, Va, Vb, VIa, VIb, VIIa or VIIb,

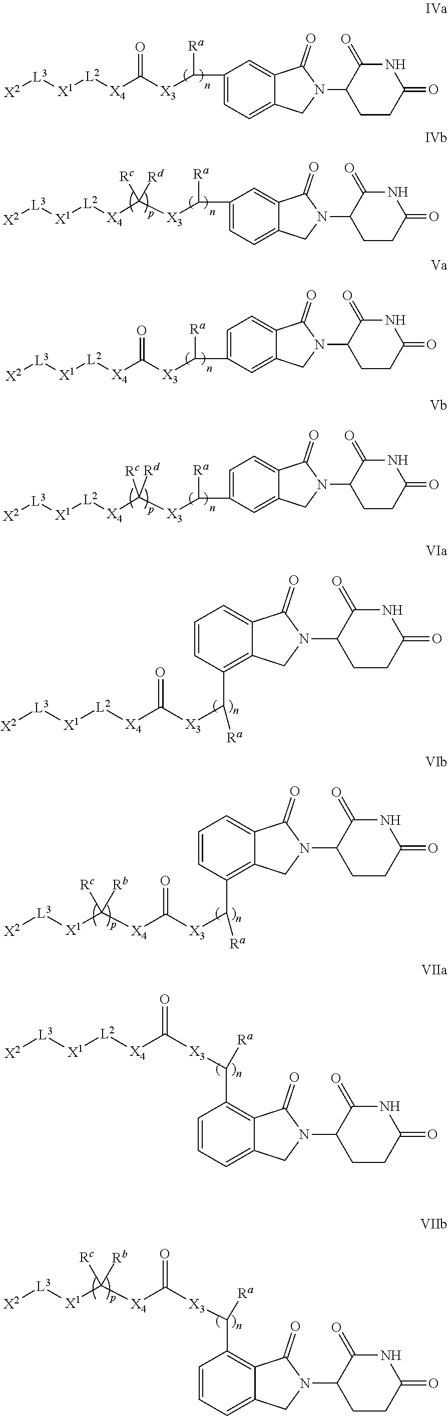

wherein X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—; X⁴ is —NH—, —CH₂—;
L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen;
R^{a} is a H or C₁₋₄ alkyl; R^{b}, R^{c} are independently of each other H, C₁₋₄ alkyl, such as methyl, ethyl, or halogen, such as F; n is 0, 1, 2; and p is 0, 1, 2.

In some embodiments, the compound of formula I or pharmaceutically acceptable salt or stereoisomer thereof is a compound of formula IXa, IXb, or IXc

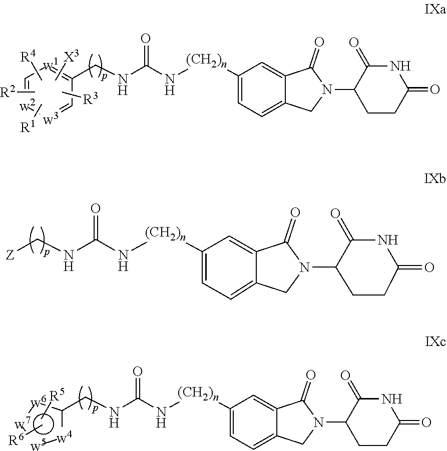

wherein in formula IXa
one of w¹, w² or w³ is selected from C and N, and the other two of w¹, w² or w³ are C;
X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁-4 alkyl-4-8 membered heterocycloalkyl, wherein X³ is unsubstituted or substituted with one or more of linear or branched C₁₋₆ alkyl, NH₂, NMe₂ or 5-6 membered heterocycloalkyl;
R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂ —O—(CH₂)₂—OMe, OCF₃, —CN, —N(H)C(O)—C₁₋₆alkyl, —OC(O)—C₁₋₄alkylamino, —OC(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; and n is 1 or 2; and p is 0, 1 or 2;
wherein in formula IXb
Z is linear or branched C₁₋₆ alkyl or C₃₋₆ cycloalkyl, C₁₋₄ alkoxy or C₁₋₄ alkyl-C₁₋₄ alkoxy, wherein Z is unsubstituted or substituted with C₁₋₄ alkyl; n is 1 or 2; and p is 0, 1 or 2
wherein in formula IXc
one or two of w⁴, w⁵, w⁶, w⁷ is selected from C, O, N, NMe, NH, or S while two or three of w⁴, w⁵, w⁶ and w⁷ are C; R⁵, R⁶ each are independently selected from hydrogen, linear or branched C₁₋₄ alkyl, CF₃, CHF₂, halogen, such as F, Cl, Br, e.g. F or Cl; and n is 1 or 2; and p is 0, 1 or 2.

In some embodiments, the compound of formula I or pharmaceutically acceptable salt or stereoisomer thereof is a compound of formula I is a compound of formula X,

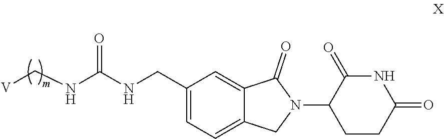

wherein m is 0, 1, 2 or 3, and
V is selected from

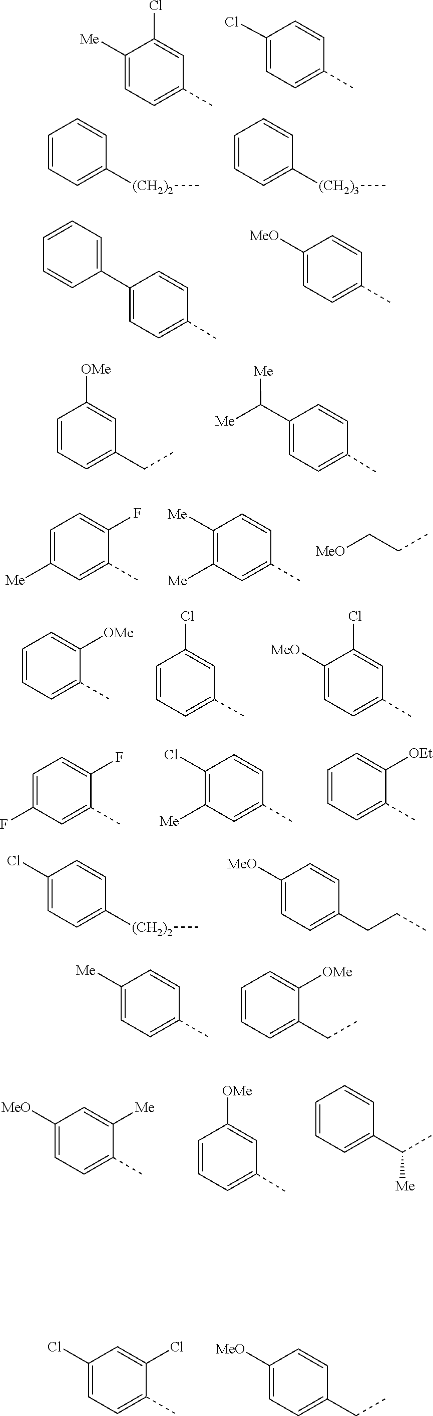

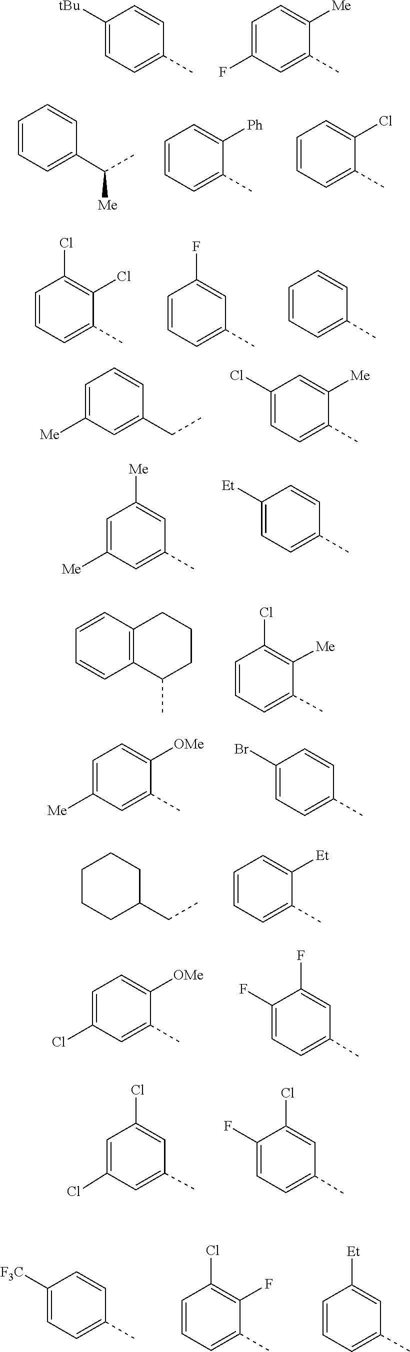

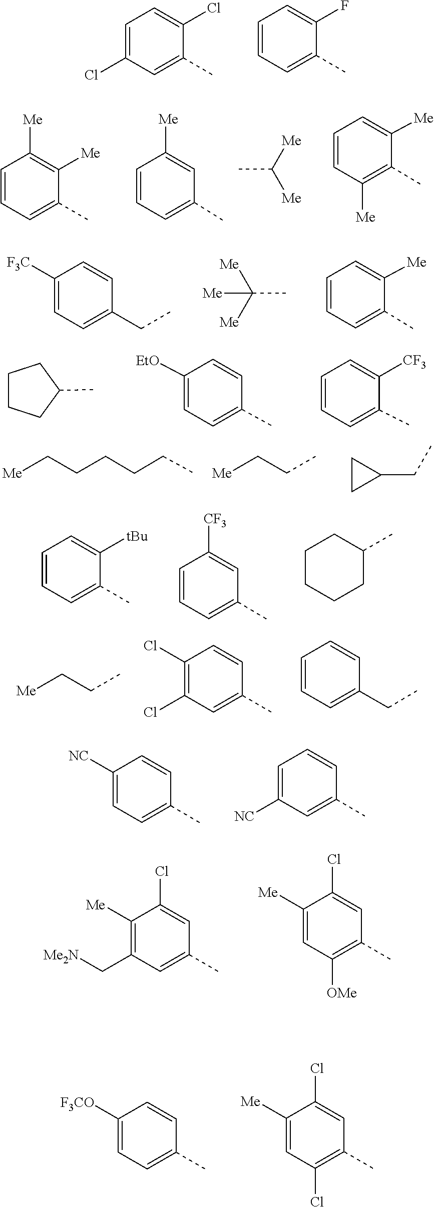

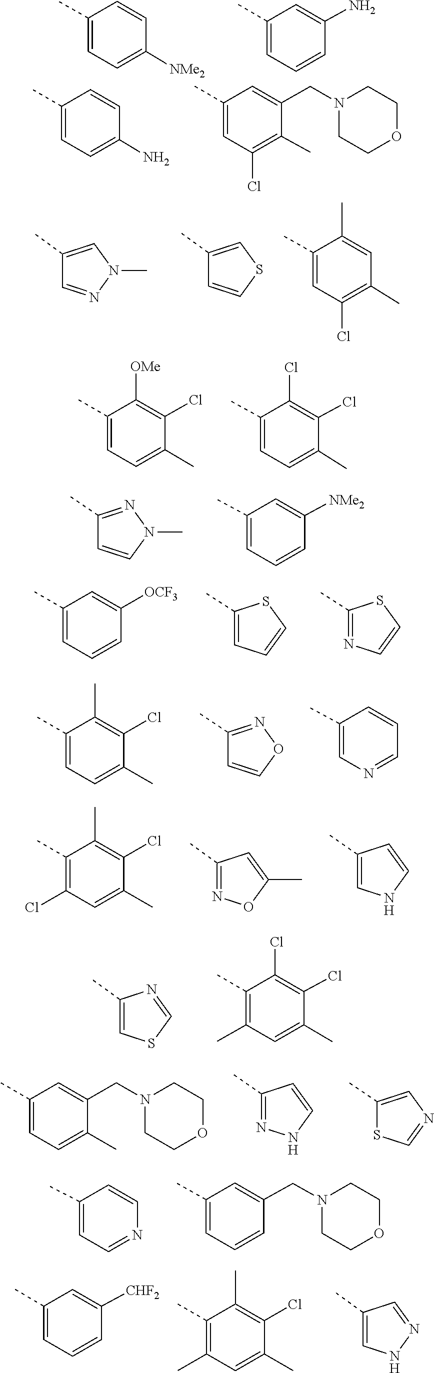

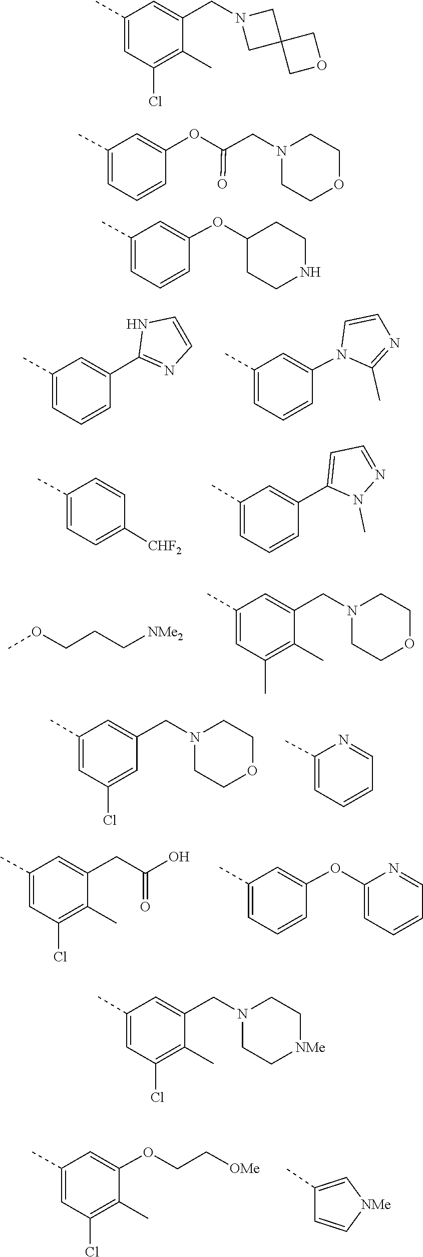

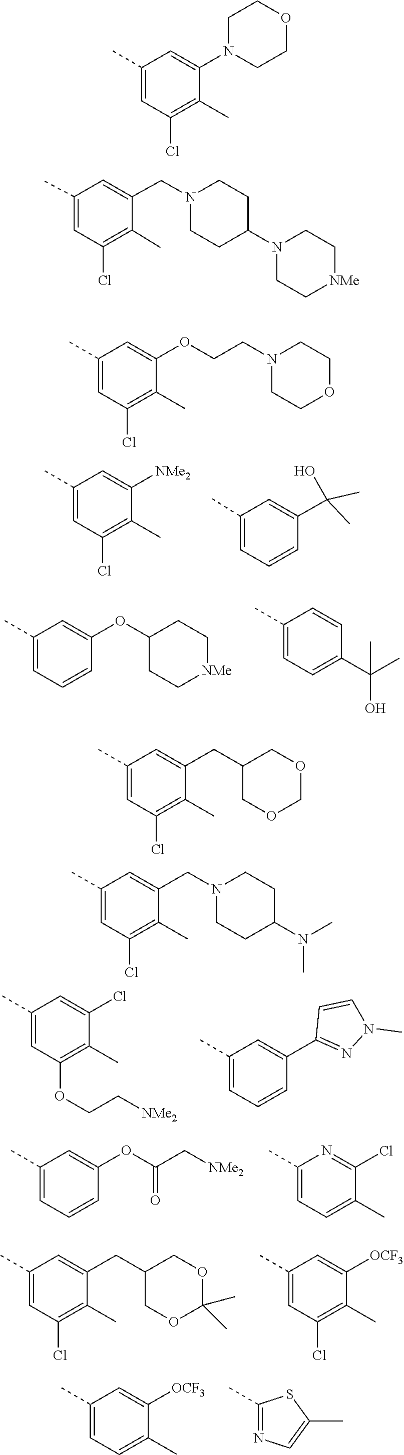

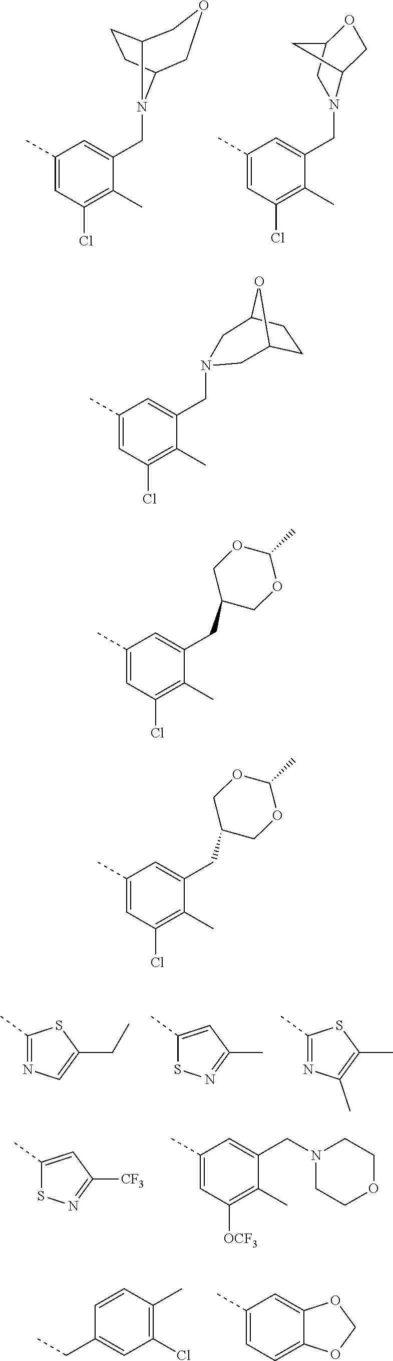

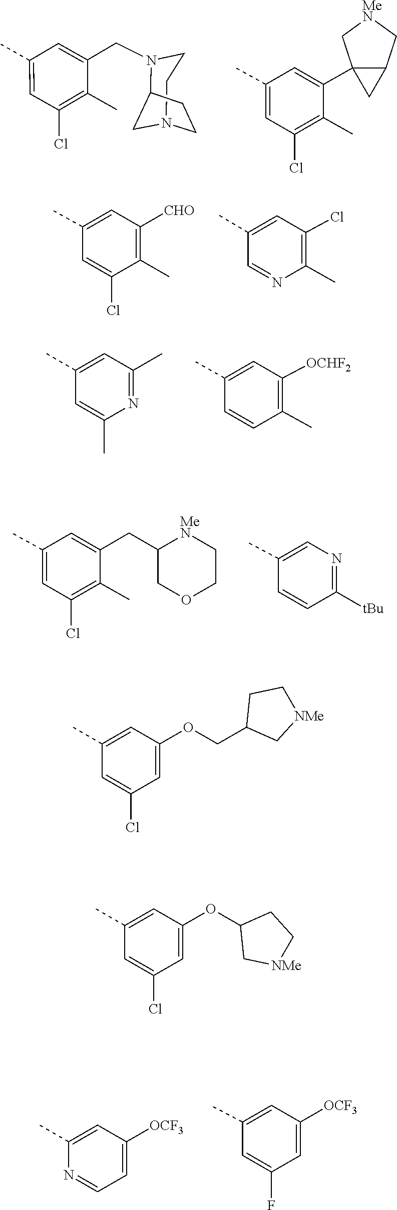

In some embodiments, the compound of formula I or pharmaceutically acceptable salt or stereoisomer thereof is a compound of formula of formula XIa, XIb, or XIc

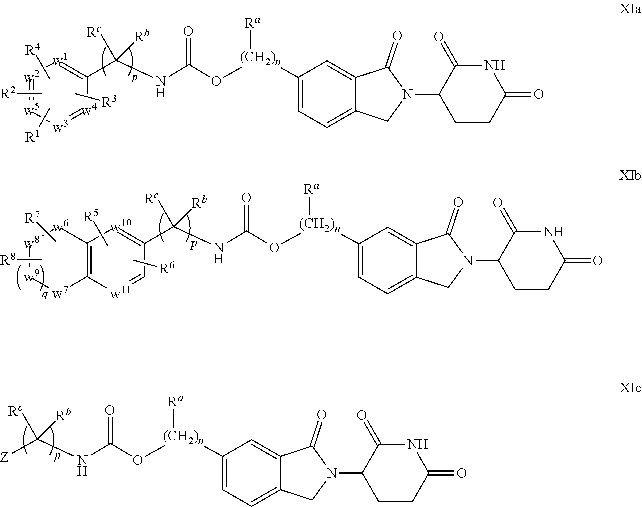

wherein in formula XIa
w¹, w², w³, w⁴, w⁵ are independently of each other selected from C and N, with the proviso that at least three of w¹, w², w³, w⁴, w⁵ are C;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl; R^{b}, R^{c} are independently of each other H, linear or branched C₁₋₄ alkyl; and n is 1, or 2, and p is 0, 1, or 2;
wherein in formula XIb
one or two of w⁶, w⁷, w⁸, w⁹ are selected from C and O and the remaining of w⁶, w⁷, w⁸, w⁹ are C; w¹⁰, w¹¹ are independently of each other selected from C and N;
R⁵, R⁶, R⁷ R⁸ are independently of each other selected from H, linear or branched C₁₋₄ alkyl, halogen, such as F or Cl, e.g. F;
R^{a} is H, linear or branched C₁₋₄ alkyl; R^{b}, R^{c} are independently of each other H, linear or branched C₁₋₄ alkyl; and q is 0 or 1; n is 1, or 2, and p is 0, 1, or 2;
wherein in formula XIc
Z is H, linear or branched —C₁₋₆ alkyl, —C₃₋₆ cycloalkyl, 4-8 membered heterocycloalkyl, wherein Z is unsubstituted or substituted with linear or branched C₁₋₄ alkyl, C₆ aryl, C₆ aryloxy, 6 membered heteroaryl or CF₃; or Z together with the N atom of the carbamate forms a 4-8 membered heterocycloalkyl, which is unsubstituted or substituted with linear or branched C₁₋₄ alkyl, C₆ aryl, C₆ aryloxy, 6 membered heteroaryl or CF₃; and
R^{a} is H, linear or branched C₁₋₄ alkyl; R^{b}, R^{c} are independently of each other H, linear or branched C₁₋₄ alkyl; and n is 1, or 2, and p is 0, 1, or 2.

In some embodiments, the compound of formula I or pharmaceutically acceptable salt or stereoisomer thereof is a compound of formula XIIa or XIIb

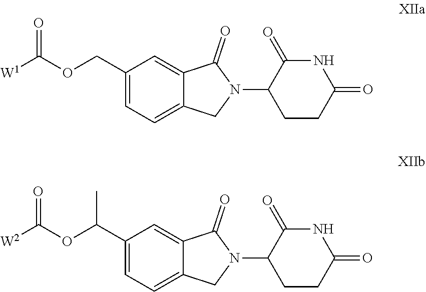

wherein W¹ and W² are selected from

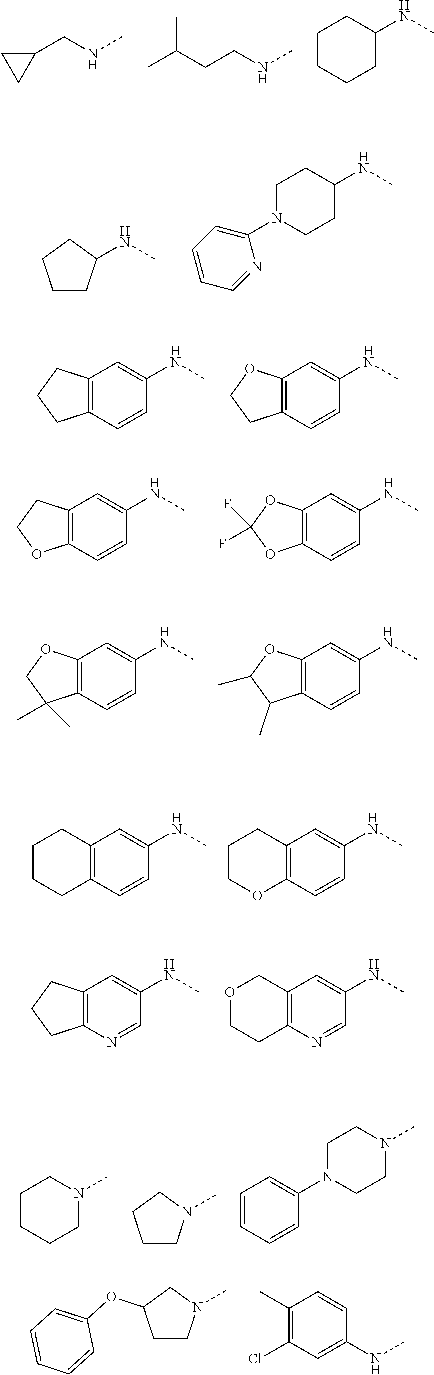

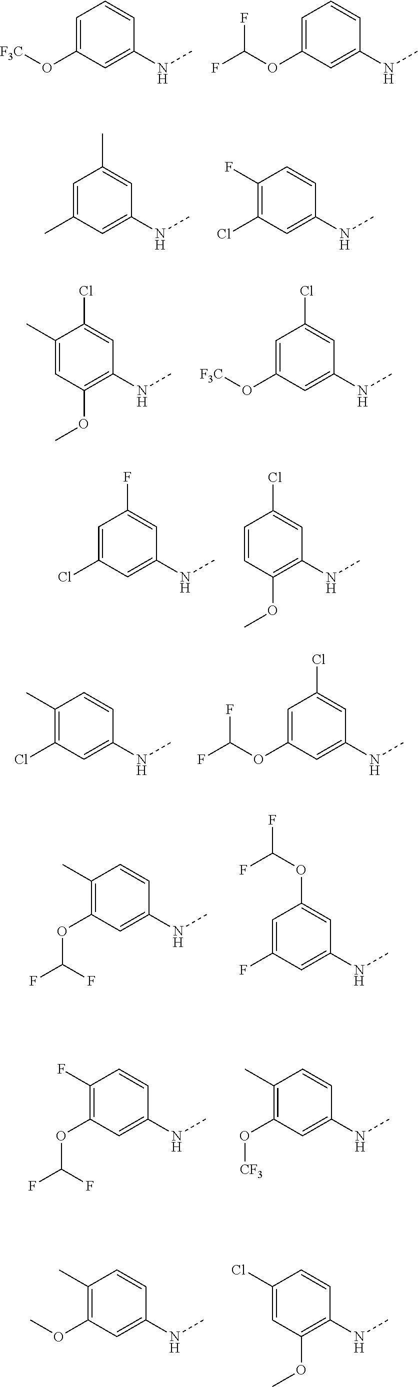

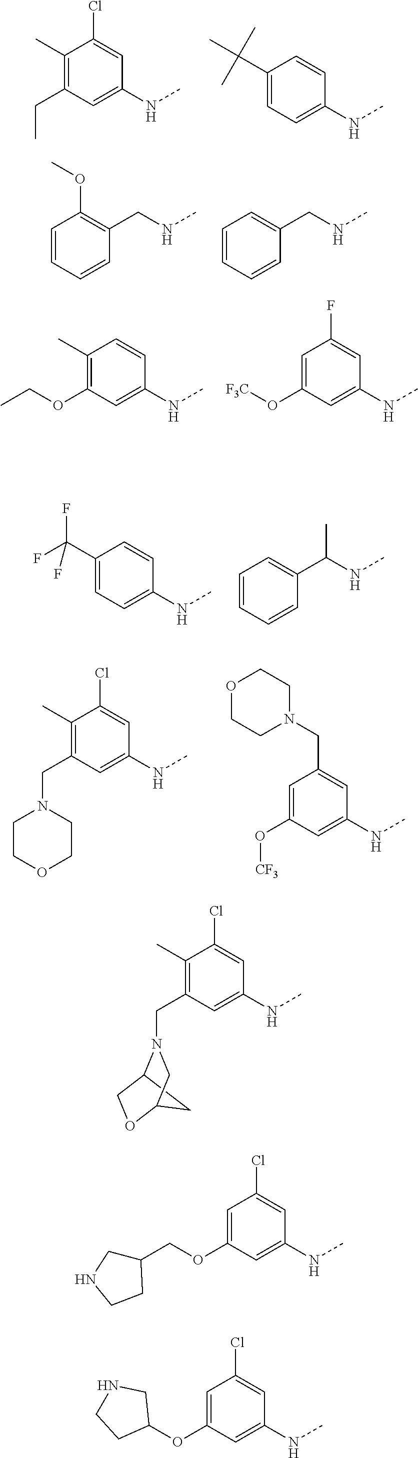

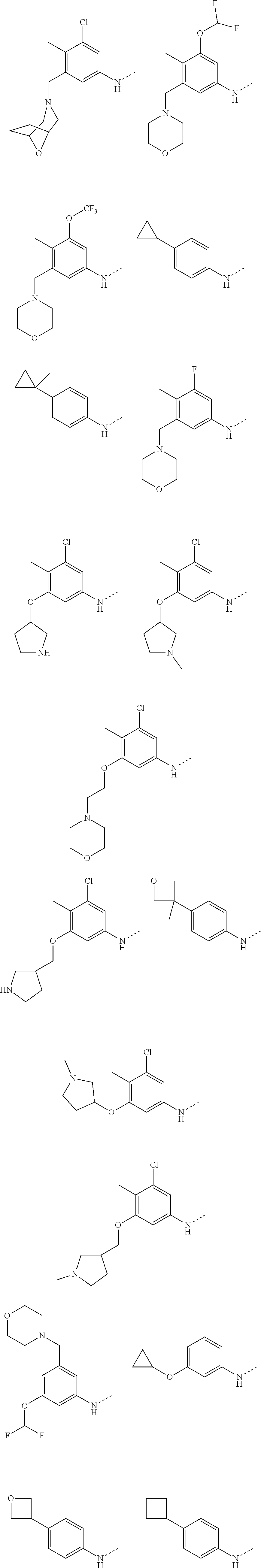

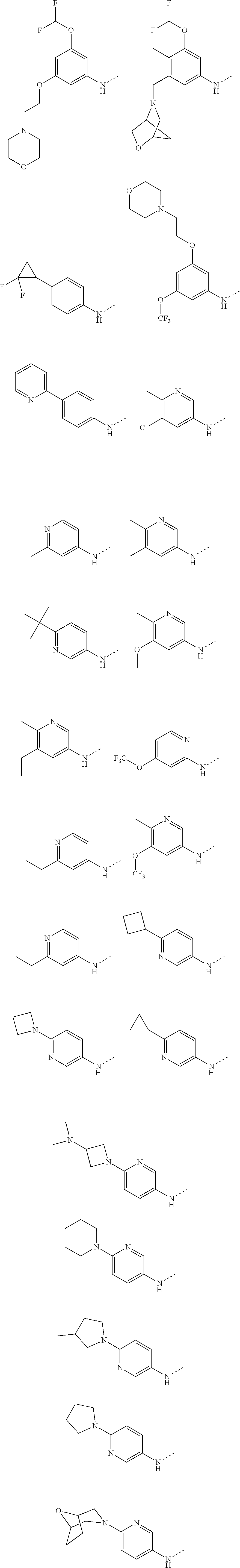

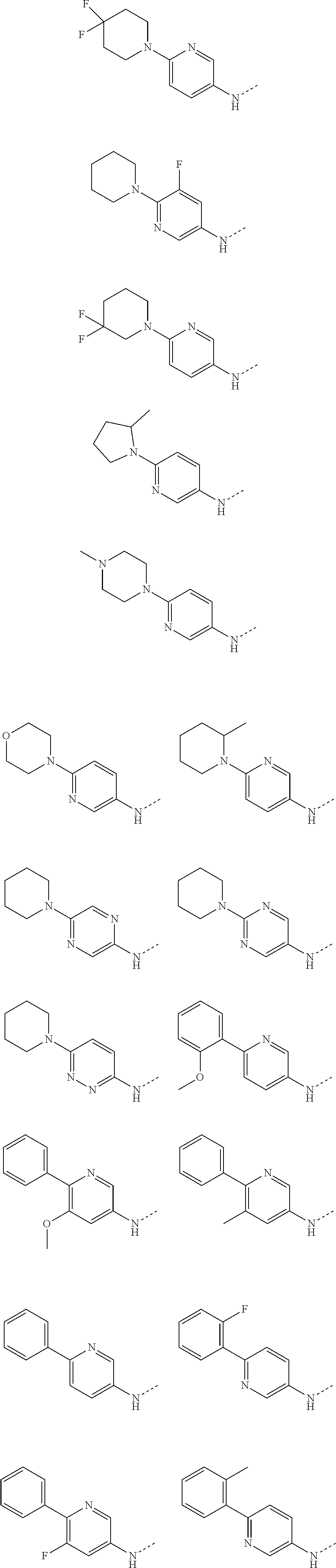

In some embodiments, the aminoglycoside used in a combined therapy with a compound of formula I or pharmaceutically acceptable salt or stereoisomer thereof is selected from geneticin, ELX-02, rhodostreptomycin, streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, paromomycin, lividomycin, apramycin, and derivatives thereof.

The present disclosure also relates to a method of preventing or treating a disease or disorder caused by or associated with a premature termination codon, the method comprising administering a compound of formula I as defined herein or a pharmaceutically acceptable salt or stereoisomer thereof to a subject in need thereof, in an amount effective for preventing or treating a disease or disorder caused by or associated with the premature termination codon.

The compound or a pharmaceutically acceptable salt or stereoisomer may be used in monotherapy or in a combined therapy with an aminoglycoside or pharmaceutically acceptable salts thereof.

In some embodiments of a combined therapy, the compound of formula I or a pharmaceutically acceptable salt or stereoisomer thereof and the aminoglycoside are administered in a simultaneous or sequential manner. In some embodiments of a combined therapy, the compound of formula I or a pharmaceutically acceptable salt or stereoisomer thereof and the aminoglycoside are in form of one single pharmaceutical composition or in form of separate pharmaceutical compositions.

In some embodiments, the disease or disorder is selected from the group consisting of, but not limited to, beta-thalassemia, Ehlers-Danlos syndrome, severe myoclonic epilepsy of infancy, achromatopsia, retinitis pigmentosa, Usher Syndrome Type 1C, adducted thumb-clubfoot syndrome, Alagille syndrome, Alstroem syndrome, antithrombin deficiency, Carney complex, Currarino syndrome, Diamond-Blackfan anemia, erythropoietic protoporphyria, Fabry disease, factor XIII deficiency, Fanconi-Bickel syndrome, fish odor syndrome, Gaucher disease, hereditary hemorrhagic telangiectasia, homocystinuria, Joubert syndrome and related disorders, Krabbe disease, L-2-hydroxyglutaric aciduria, methylmalonic academia, Niemann-Pick disease, Peters plus syndrome, Townes-Brocks disease, von Willebrand disease, Wiskott-Aldrich syndrome, Kabuki syndrome, Chanarin-Dorfman syndrome, lecithin:cholesterol acyltransferase deficiency/fish-eye disease, Marfan Syndrome, mucopolysaccharidiosis, amyloidiosis, Late Infantile Neuronal Ceroid Lipofuscinosis, coenzyme Q10 Deficiency, peroxisome biogenesis disorders, lysosomal storage disorders, colorectal cancer, congenital enteropeptidase deficiency, cystic fibrosis, Hungarian Peutz-Jeghers Syndrome, Jervell and Lange-Nielsen syndrome, Lynch syndrome, microvillus inclusion disease, Peutz-Jeghers syndrome, xanthinuria, acidosis, Alport syndrome, Bardet-Biedl syndrome, Birt-Hogg-Dube syndrome, Dent's disease, Gitelman syndrome, hereditary leiomyomatosis and renal cell cancer, hereditary spherocytosis, leber congenital amaurosis, lysinuric protein intolerance, nephronophthisis, polycystic kidney disease, pseudohypoaldosteronism, renal hypodysplasia, sporadic clear cell renal cell carcinoma, type 2 papillary renal cell cancers, urofacial syndrome, von Hippel-Lindau disease, Wilms' tumor, X-linked Alport syndrome, X-linked hypophosphatemic rickets, hyperuricaemic nephropathy (juvenile/medullary cystic kidney disease), tuberous sclerosis, nephrotic syndrome/congenital nephrotic syndrome, Finnish type nephrotic syndrome, steroid resistant nephrotic syndrome 3, early onset nephrotic syndrome/Pierson syndrome, Denys-Drash syndrome, nephrotic syndrome/Schimke immuno-osseous dysplasia, primary glucocorticoid resistance, X-linked hypophosphatemia, primary hyperoxaluria type 1, pseudohypoaldosteronism type 1, proximal renal tubular acidosis, abetalipoproteinemia and homozygous familial hypobetalipoproteinemia, Alpers syndrome, carbamyl phosphate synthetase I deficiency, cholesteryl ester storage disease, citrin deficiency, Dubin-Johnson syndrome, erythropoietic protoporphyria, factor V deficiency, glycogen storage disease, Hemophilia A (factor VIII Deficiency), Hemophilia B (factor IX Deficiency), hepatocellular carcinoma, hepatoerythropoietic porphyria, hereditary spastic paraplegias, hypobetalipoproteinemia, inherited factor XI deficiency, diabetes mellitus (Type 1 and Type 2), microcytic anemia and iron deficiency, mitochondrial DNA depletion, mitochondrial DNA depletion syndrome, phenylketonuria, polycystic liver disease, porphyria cutanea tarda, progressive familial intrahepatic cholestasis, Wilson Disease, autosomal dominant hypercholesterolemia, factor XII deficiency, factor X deficiency, hypofibrinogenaemia, afibrinogenaemia, factor VII deficiency, agammaglobulinemia, amegakaryocytic thrombocytopenia, dyserythropoietic anemia type II, Duchenne and Becker muscular dystrophy, centronuclear myopathies, limb girdle muscular dystrophy or Miyoshi myopathy, Ullrich disease, spinal muscular atrophy, dystrophic epidermolysis bullosa, Hailey-Hailey Disease, Herlitz junctional epidermolysis bullosa, Netherton syndrome, ataxia-telangiectasia, Dravet syndrome, myotonic dystrophy, infantile neuronal ceroid lipofuscinosis, Alzheimer's disease, Tay-Sachs disease, neural tissue degeneration, Parkinson's disease, lupus erythematosus, graft-versus-host disease, severe combined immunodeficiency, DNA Ligase IV deficiency, Nijmegen breakage disorders, xeroderma pigmentosum (XP), familial erythrocytosis, nephrolithiasis, osteogenesis imperfect, cirrhosis, neurofibroma, bullous disease, lysosomal storage disease, Hurler's disease, familial cholesterolemia; cerebellar ataxia; lung disease; cystic fibrosis; pigmentary retinopathy; amyloidosis, atherosclerosis, gigantism, dwarfism, hypothyroidism, hyperthyroidism, obesity.

In some embodiments, the disease or disorder is cancer. In some embodiments, the disease or disorder is cystic fibrosis. In some embodiments, the disease or disorder is DMD (dystrophin).

The present disclosure is also related to a method of promoting readthrough of a premature termination codon, comprising providing a compound of formula I as defined herein or a pharmaceutically acceptable salt or stereoisomer thereof; optionally in combination with an aminoglycoside as defined herein.

The present disclosure further relates to a pharmaceutical combination comprising or consisting of:
(a) a compound of formula I or a pharmaceutically acceptable salt or stereoisomer thereof

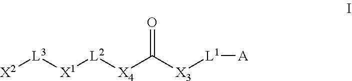

wherein A is

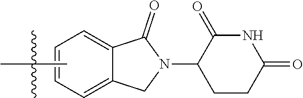

X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆ alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—; X⁴ is —NH—, —CH₂—;
L¹ is a covalent bond, C₁₋₆ alkyl, which is unsubstituted or substituted with one or more of C₁₋₄ alkyl, halogen; L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen, and
(b) an aminoglycoside or a pharmaceutically acceptable salt thereof

In some embodiments, the ratio of the compound of formula I to the aminoglycoside is between 100:1 to 1:100 (w/w), such as 50:1 to 1:50 (w/w), e.g. 10:1 to 1:10 (w/w).

The pharmaceutical combination of the disclosure may be used in the prevention and treatment of a disease or disorder caused by or associated with one or more premature termination codons.

The disclosure further relates to a kit comprising a dosage form of a compound of formula I, or a pharmaceutically acceptable salt thereof, and instructions for the use in a monotherapy as defined herein. In some embodiments, the kit further comprises a dosage form of an aminoglycoside, and instructions for the use in a combined therapy as defined herein.

### BRIEF DESCRIPTION OF THE FIGURES

FIG. 1A, FIG. 1B, and FIG. 1C show GSPT1 degradation in the three cancer cell lines DMS-114, ESS-1 and HDQ-P1 (each harboring the R213X nonsense mutation) in the absence or presence of a representative compound of the disclosure (A), CC-885 (B) and CC-90009 (C). The cells were treated with the indicated compound in concentrations ranging from 0.03 to 30 μM for 24 hours. Cells were lysed and GSPT1 levels were measured by WES.

FIG. 2A, FIG. 2B, and FIG. 2C show TP53 readthrough in cancer cell lines harboring the R213X nonsense mutation. PTC readthrough in cancer cell lines DMS-114, ESS-1 and HDQ-P1 following treatment with a representative compound of the disclosure (A), CC-885 (B) and CC-90009 (C) in the absence or presence of geneticin (G418). Cells were exposed to the indicated compound in concentrations ranging from 0.03 to 3 μM without or with 10 or 20 μg/mL G418 for 48 hr and TP53 levels were measured. Vinculin was used as a loading control. The chemiluminescence signal was plotted for each compound concentration in the presence or absence of G418.

FIG. 3A, FIG. 3B, and FIG. 3C show the effect of a representative compound of the disclosure on CFTR readthrough: Readthrough of the common G542X cystic fibrosis transmembrane conductance regulator (CFTR) nonsense mutation was carried out in the immortalized human bronchial epithelial cells HBE16. A HaloTag G542X 5×5 NanoLuc reporter assay was established in that cell line containing the CFTR readthrough cassette bearing the G542X codon as well as 5 codons upstream and downstream from the human CFTR sequence. Cells were treated with a representative compound of the disclosure at indicated concentrations ranging from 0.03 to 0.26 μM (representing GSPT1 DC₃₀, DC₄₀ and DC₅₀ values in that cell line after 24 hr) without or with geneticin (G418, used at 15 μM corresponding to EC10). Data is represented as (A) NanoLuc activity (readthrough activity) as fold-change with y-axis left indicating the readthrough activity in Nano RLU/μg protein, and y-axis right indicating readthrough/foldchange or (B) relative to G418 EC50 (35 μM) with y-axis left indicating the readthrough activity in Nano RLU/μg protein, and y-axis right indicating readthrough/% G418 EC50. The total protein assay (C) indicates no significant decrease of HBE16 cell viability following treated with the representative compound of the disclosure alone or in combination with G418 (y-axis left indicating protein μg/μL, and y-axis right indicating %).

FIG. 4A, FIG. 4B, and FIG. 4C show the effect of a compound of the disclosure on CFTR readthrough: Assay was performed as described for FIG. 3A, FIG. 3B, and FIG. 3C with cells being treated with a representative compound of the disclosure at indicated concentrations ranging from 0.03 to 0.26 μM (representing GSPT1 DC₃₀, DC₄₀ and DC₅₀ values in that cell line after 24 hr) without or with ELX-02 (used at 85 μM corresponding to EC10). Data is represented as (A) NanoLuc activity (readthrough activity) as fold-change with y-axis left indicating the readthrough activity in Nano RLU/ug protein, and y-axis right indicating readthrough/foldchange or (B) relative ELX-02 G418 EC50 (250 μM) with y-axis left indicating the readthrough activity in Nano RLU/ug protein, and y-axis right indicating readthrough/% ELX-02 EC50. The total protein assay (C) indicates no significant decrease of HBE16 cell viability following treated with the representative compound of the disclosure alone or in combination with ELX-02 (y-axis left indicating protein μg/μL, and y-axis right indicating %).

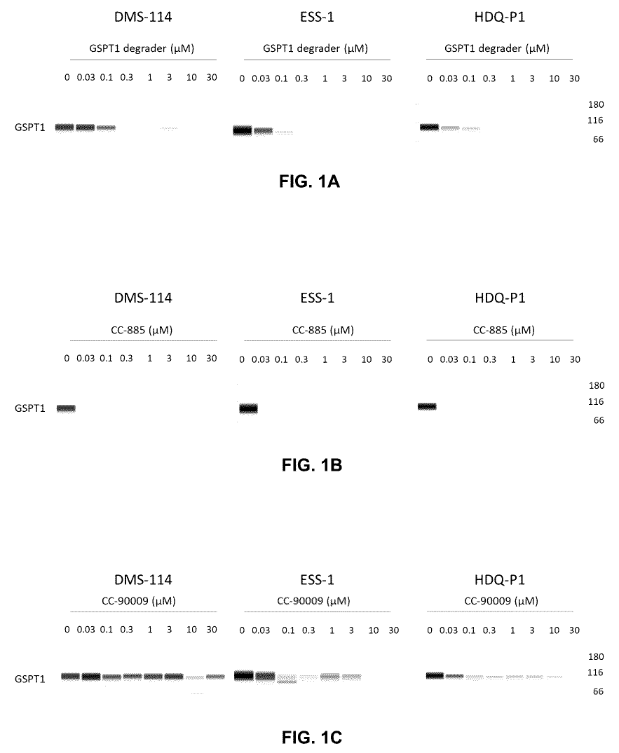

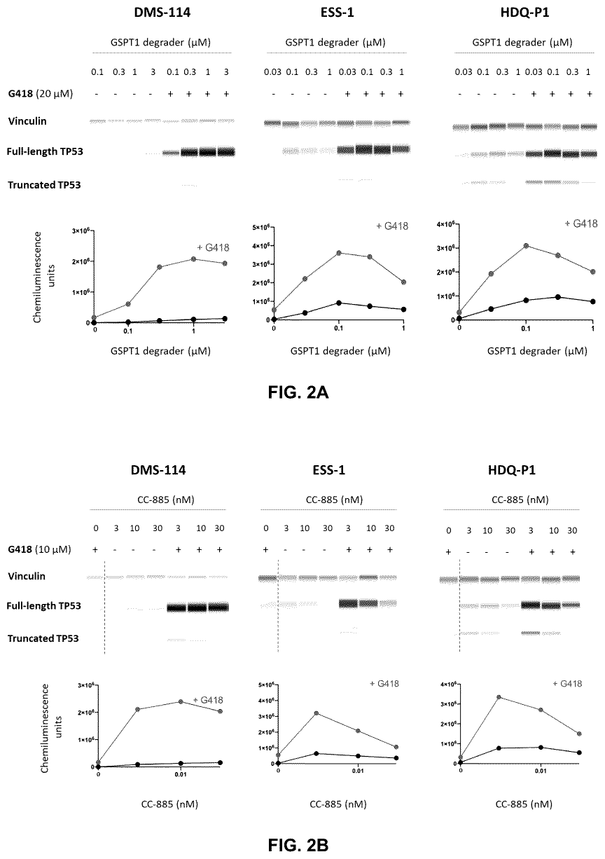

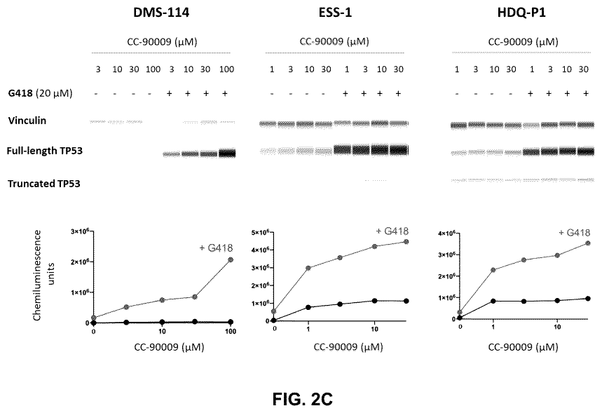

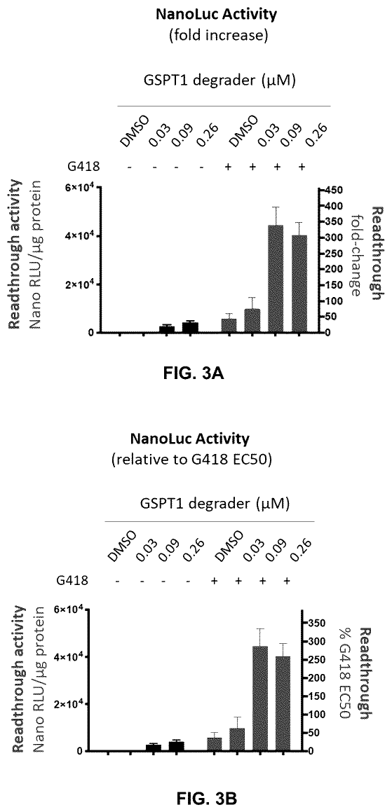

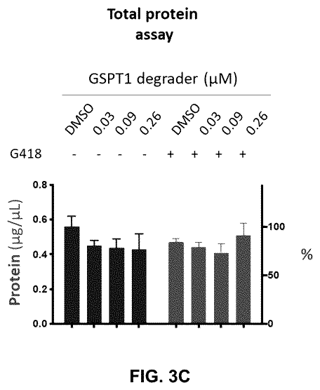

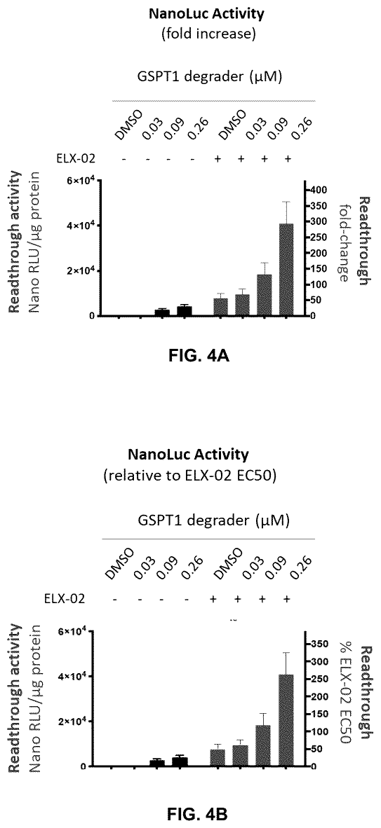

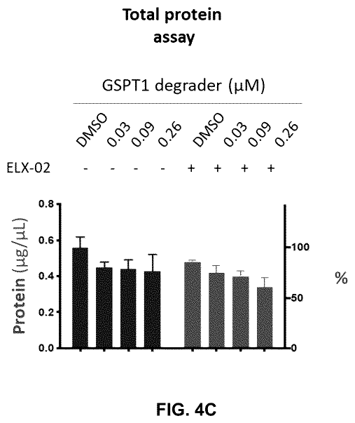

### DETAILED DESCRIPTION OF THE DISCLOSURE

Unless specified otherwise the following general definitions apply to all compounds of the disclosure and aminoglycosides according to the disclosure.

The term “compound of the disclosure” as used herein, refers to any compounds of formula I to XII, including any of the specific examples disclosed herein, as well as pharmaceutically acceptable salts and/or stereoisomers thereof.

The term “aminoglycoside” or “aminoglycoside of the disclosure” as used herein, refers to any aminoglycoside of the prior art and in particular to any aminoglycoside as defined in the description, as well as pharmaceutically acceptable salts and/or stereoisomers thereof.

The term “active agent(s)”, “active agent(s) of the disclosure” or “active ingredient(s) of the disclosure” refers to at least one compound of formula I and/or at least one aminoglycoside as defined herein.

It is understood that “independently of each other” means that when a group is occurring more than one time in any compound, its definition on each occurrence is independent from any other occurrence. It is further understood that a dashed line (or a wave being transverse to a bond) or a solid line without attachment, such as —C₁₋₄ alkyl, depicts the site of attachment of a residue (i.e. a partial formula). It is further understood that the abbreviations “C” and “N” are representative for all possible degrees of saturation, which typically do not result in radicals, nitrenes or carbenes, i.e. N includes —NH— and —N═, C includes —CH₂— and ═CH—. In addition, “C” as an atom in an aromatic or heteroaromatic ring which has a substituent R^{x} at any suitable position, includes ═CH— as well as ═CR^{x}—.

Based on the definitions given throughout the application the skilled person knows, which combinations are synthetically feasible and realistic, e.g. typically combinations of groups leading to some heteroatoms directly linked to each other, e.g. —O—O—, are not contemplated, however synthetically feasible combinations, such as —S—N═ in an isothiazole are contemplated.

The term “saturated” in reference to ring systems refers to a ring having no double or triple bonds. The term “partially unsaturated” in reference to ring systems refers to a ring that includes at least one double or triple bond, but does not include aromatic systems.

The term “aromatic” refers to monocyclic or multicyclic (e.g. bicyclic) ring systems, which show some or complete conjugation or delocalization of their electrons. Aromatic monocyclic rings, such as aryl or heteroaryl rings as defined herein, include phenyl, pyridinyl, furyl and the like. Aromatic multicyclic rings, such as aryl or heteroaryl rings as defined herein, refer to ring systems, wherein at least one ring is an aromatic ring, and thus include (i) aromatic ring systems, wherein an aromatic ring is fused to one or more aromatic rings, such as in e.g. naphthyl, indolyl, benzimidazolyl, and the like (also referred to as fully aromatic ring systems), and (ii) aromatic ring systems, wherein an aromatic ring is fused to one or more non-aromatic rings, such as in e.g. indanyl, indenyl, phthalimidyl, naphthimidyl, phenanthridinyl, tetrahydronaphthyl, 1,4-dihydronapthyl, and the like (also referred to as partially aromatic ring systems).

The term “non-aromatic” refers to (i) fully saturated rings such as monocyclic rings, e.g. cyclohexyl, and bicyclic rings, e.g. tetrahydronaphthyl, and (ii) partially unsaturated rings such as monocyclic rings, e.g. cyclohexenyl, and bicyclic rings, e.g. 1,4-dihydronapthyl.

The term “C₆₋₁₀ aryl” includes both fully aromatic C₆₋₁₀ aryl and partially aromatic C₆₋₁₀ aryl having 6, 7, 8, 9, or 10 ring atoms and includes monocycles and fused bicycles. Examples of fully aromatic C₆₋₁₀ aryl include e.g. phenyl (fully aromatic C₆ aryl), naphthyl (fully aromatic C₁₀ aryl). Examples of partially aromatic C₆₋₁₀ aryl include e.g. indenyl (partially aromatic C₉ aryl), 2,3-dihydroindenyl (partially aromatic C₉ aryl), 1, 2, 3, 4-tetrahydronaphthyl (partially aromatic C₁₀ aryl). In some embodiments for group X¹, C₆₋₁₀ aryl is phenyl, 2,3-dihydroindenyl. In some embodiments for group X², C₆₋₁₀ aryl is phenyl. The term “—C₁₋₆ alkyl-C₆₋₁₀ aryl” refers to -L²-X¹— or L³-X²— with L², L³ being a C₁₋₆ alkyl group and X¹, X² being a C₆₋₁₀ aryl, and thus refers to a C₆₋₁₀ aryl, which is linked through a C₁₋₆ alkyl group as defined herein to its neighbouring group. The term “—C₁₋₆ alkoxy-C₆₋₁₀ aryl” refers to -L²-X¹— or L³-X²— with L², L³ being a C₁₋₆ alkoxy group and X¹, X² being a C₆₋₁₀ aryl, and thus refers to a C₆₋₁₀ aryl, which is linked through a C₁₋₆ alkoxy group as defined herein to its neighbouring group. The term “—O—C₆₋₁₀ aryl” or “C₆₋₁₀ aryloxy” refers to -L²-X¹— or L³-X²— with L², L³ being —O— and X¹, X² being a C₆₋₁₀ aryl, and thus refers to a C₆₋₁₀ aryl, which is linked through a —O— group to its neighbouring group. The C₆₋₁₀ aryl group may be unsubstituted or substituted with C₁₋₄ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl.

In some embodiments for X¹, a C₆₋₁₀ aryl group refers to a fully aromatic ring system, e.g. phenyl, which is unsubstituted or substituted with C₁₋₄ alkyl, such as methyl, ethyl, t-butyl, fluorinated C₁₋₄ alkyl, such as —C(CH₃)F₂, C₁₋₄ alkoxy, such as methoxy, ethoxy, fluorinated C₁₋₄ alkoxy, such as —OCF₃, —OCHF₂, CN, halogen, such as F or Cl, In some embodiments for X¹, a C₆₋₁₀ aryl group refers to a partially aromatic ring system, e.g. 2,3-dihydroindenyl, which is unsubstituted or substituted with C₁₋₄ alkyl, such as methyl, ethyl, t-butyl, or halogen, such as F or Cl.

In some embodiments for X², a C₆₋₁₀ aryl group refers to a fully aromatic ring system, e.g. phenyl, which is unsubstituted or substituted with C₁₋₄ alkyl, such as methyl, ethyl, C₁₋₄ alkoxy, such as methoxy, ethoxy, halogen, such as F, Cl, or Br, such as F or Cl, e.g. F.

The term “5-10 membered heteroaryl” refers to a fully or partially aromatic ring system in form of monocycles or fused bicycles having 5, 6, 7, 8, 9, 10 ring atoms selected from C, N, O, and S, such as C, N, and O, or C, N, and S, with the number of N atoms being e.g. 0, 1, 2 or 3 and the number of O and S atoms each being 0, 1 or 2. In some embodiments a 5-10 membered heteroaryl refers to a fully aromatic ring system having 5, 6, 7, 8, 9, 10, such as or 6, e.g. 6 ring atoms selected from C and N, with the number of N atoms being 1, 2 or 3, such as 1 or 2. In some embodiments a 5-10 membered heteroaryl refers to a fully aromatic ring system having 5, 6, 7, 8, 9, 10, such as 5 or 6, e.g. 5 ring atoms selected from C, N, O, S with the number of N, S and O atoms each being independently 0, 1 or 2. In some embodiments the total number of N, S and O atoms is 2. In some embodiments a 5-10 membered heteroaryl refers to a fully aromatic ring system having 5 ring atoms selected from C, N, S with the number of N and S atoms each being independently 0 or 1. In some embodiments the total number of N and S atoms is 2. In some embodiments a 5-10 membered heteroaryl refers to a fully aromatic ring system having 6 ring atoms selected from C and N, with the number of N atoms being 1 or 2. In other embodiments a 5-10 membered heteroaryl refers to a partially aromatic ring system having 9 or 10 ring atoms selected from C, N and O, with the number of O atoms being 1, 2 or 3, such as 1 or 2, and the number of N atoms being 1 or 2, such as 1. In some embodiments, examples of “5-10 membered heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiophenyl, thiazolyl, thienyl, indolyl, quinazolinyl, oxazolinyl, isoxazolinyl, indazolinyl, isothiazolyl, 1,3-benzodioxolyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, benzodihydropyrane, 1,2,3,4-tetrahydronaphthyl, 2,3-dihydroindenyl and the like. In some embodiments, examples of “5-10 membered heteroaryl” include 5-10 membered heteroaryl, such as isothiazole, 6-membered heteroaryl, such as pyridinyl, pyrimidinyl, pyridazinyl, pyrazinyl, 9-membered heteroaryl, such as 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, and 10-membered heteroaryl, such as benzodihydropyrane (chromane), dihydropyrano-pyridine. The term “—C₁₋₆ alkyl 5-10 membered heteroaryl” refers to -L²-X¹— or L³-X²— with L², L³ being a C₁₋₆ alkyl group and X¹, X² being a 5-10 membered heteroaryl, and thus refers to a 5-10 membered heteroaryl, which is linked through a C₁₋₆ alkyl group as defined herein to its neighbouring group. The term “—C₁₋₆ alkoxy 5-10 membered heteroaryl” refers to -L²-X¹— or L³-X²— with L², L³ being a C₁₋₆ alkoxy group and X¹, X² being a 5-10 membered heteroaryl, and thus refers to a 5-10 membered heteroaryl, which is linked through a C₁₋₆ alkoxy group as defined herein to its neighbouring group. The term “—O-5-10 membered heteroaryl” refers to -L²-X¹— or L³-X²— with L², L³ being —O— and X¹, X² being a 5-10 membered heteroaryl, and thus refers to a 5-10 membered heteroaryl, which is linked through a —O— group to its neighbouring group. The 5-10 membered heteroaryl group may be unsubstituted or substituted with C₁₋₄ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl. In some embodiments, the 5-10 membered heteroaryl group may be unsubstituted or substituted with C₁₋₄ alkyl, such as methyl, ethyl, t-butyl, fluorinated C₁₋₄ alkyl, such as —CF₃, C₁₋₄ alkoxy, such as methoxy, ethoxy, halogen, such as F or Cl.

In some embodiments for X¹, a 5-10 membered heteroaryl refers to a fully aromatic ring system having 5 ring atoms selected from C, N and S with the number of N and S atoms being independently of each other 0 or 1, e.g. 1 or a fully aromatic ring system having 6 ring atoms selected from C and N, with the number of N atoms being 1 or 2 or a partially aromatic ring system having 9 or 10 ring atoms selected from C, N and O, with the number of 0 atoms being 1 or 2 and the number of N atoms being 1. In some embodiments for X¹, a 5-10 membered heteroaryl refers to isothiazole, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine.

In some embodiments for X² a 5-10 membered heteroaryl refers to a fully aromatic ring system having 6 ring atoms selected from C and N, with the number of N atoms being 1 or 2, such as 1. In some embodiments for X² a 5-10 membered heteroaryl refers to pyridinyl.

The term “C₃₋₆ cycloalkyl” refers to a non-aromatic, i.e. saturated or partially unsaturated alkyl ring system, such as monocycles, fused bicycles, bridged bicycles or spirobicycles, containing 3, 4, 5 or 6 carbon atoms. Examples of “C₃₋₈ cycloalkyl” include monocycles, such as cyclopropyl, cyclobutyl, cyclopentyl, cyclohexyl, bridged bicycles, such as bicyclo[1.1.1]pentyl, bicyclo[2.1.1]hexyl, fused bicycles, such as bicyclo[3.1.0]hexyl. The C₃₋₆ cycloalkyl group may be unsubstituted or substituted with C₁₋₄ alkyl, such as methyl, ethyl, t-butyl, fluorinated C₁₋₄ alkyl, such as —CF₃, —C(CH₃)F₂, C₁₋₄ alkoxy, such as methoxy, ethoxy, fluorinated C₁₋₄ alkoxy, such as —OCF₃, —OCHF₂, CN, —N(Me)₂, halogen, such as F, Cl, or Br, such as F or Cl. In some embodiments the C₃₋₆ cycloalkyl group may be unsubstituted or substituted by e.g. one or more of C₁₋₄ alkyl, such as methyl and halogen, such as F. The term “—C₁₋₄ alkyl-C₃₋₆ cycloalkyl” refers to -L²-X¹— or L³-X²— with L², L³ being a C₁₋₄ alkyl group and X¹, X² being C₃₋₆ cycloalkyl as defined herein and refers to a C₃₋₆ cycloalkyl, which is linked through a C₁₋₆ alkyl group as defined herein to its neighbouring group. The term “—O—C₃₋₆ cycloalkyl” refers to -L²-X¹— or L³-X²— with L², L³ being —O— and X¹, X² being C₃₋₆ cycloalkyl as defined herein and refers to a C₃₋₆ cycloalkyl, which is linked through —O— to its neighbouring group. The term “—C₁₋₄ alkoxy-C₃₋₆ cycloalkyl” refers to -L²-X¹— or L³-X²— with L², L³ being a C₁₋₄ alkoxy group and X¹, X² being C₃₋₆ cycloalkyl as defined herein and refers to a C₃₋₆ cycloalkyl, which is linked through a C₁₋₆ alkoxy group as defined herein to its neighbouring group. In some embodiments for X¹, a C₃₋₆ cycloalkyl refers to cyclopropyl, cyclopentyl, cyclohexyl. In some embodiments for X², a C₃₋₆ cycloalkyl refers to cyclopropyl, cyclobutyl.

The term “4-8 membered heterocycloalkyl” refers to a non-aromatic, i.e. saturated or partially unsaturated ring system having 4, 5, 6, 7 or 8 ring atoms (of which at least one is a heteroatom), which ring atoms are selected from C, N, O, and S, such as C, N, and O, the number of N atoms being 0, 1, or 2 and the number of O and S atoms each being 0, 1, or 2. In some embodiments the term “4-8 membered heterocycloalkyl” comprises saturated or partially unsaturated monocycles, fused bicycles, bridged bicycles or spirobicycles. In some embodiments the term “4-8 membered heterocycloalkyl” comprises fully saturated or partially unsaturated monocycles and bridged bicycles. Examples of 4-8 membered heterocycloalkyl groups include azetidinyl, oxetanyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxanyl, 1,3-dithianyl, piperazinyl, thiomorpholinyl, piperidinyl, morpholinyl, 1,4-diazabicyclo[3.2.1]octan-4-yl, 3-methyl-3-azabicyclo[3.1.0]hexan-1-yl, 2-oxa-5azabicyclo[2.2.1]heptan-5-yl, 8-oxa-3-azabicyclo[3.2.1]octan-3-yl and the like. The 4-8 membered heterocycloalkyl group may be unsubstituted or substituted with C₁₋₄ alkyl, such as methyl, ethyl, C₁₋₄ alkoxy, such as methoxy, ethoxy, halogen, such as F, Cl or Br, e.g. F or Cl.

In some embodiments, the 4-8 membered heterocycloalkyl representing group X¹ is a non-aromatic ring system having 5 or 6 ring atoms of which at least one is a heteroatom selected from N and O, the number of N atoms being 1 or 2 and the number of O being 0, 1, or 2, such as a non-aromatic 6 membered ring system having 1 or 2 N-atoms, such as piperidine. In some embodiments, the 4-8 membered heterocycloalkyl formed by groups X¹ together with the N atom of the carbamate forms is a non-aromatic ring system having 5 or 6 ring atoms of which at least one is a heteroatom selected from N and O, the number of N atoms being 1 or 2 and the number of O being 0, 1, or 2, e.g. a non-aromatic ring system having 5 or 6 ring atoms comprising one or two N-atoms. Examples include pyrrolidinyl, piperdinyl, morpholinyl, piperazinyl, N-methyl piperazinyl. In some embodiments, the 4-8 membered heterocycloalkyl representing X² is a non-aromatic ring system having 4, 5, 6, 7 or 8 ring atoms of which at least one is a heteroatom selected from N and O, the number of N atoms being 1 or 2 and the number of O being 0, 1, or 2. In some embodiments, 4-8 membered heterocycloalkyl include 4-membered heterocycloalkyl having at least one heteroatom selected from N and O, the number of N atoms being 1 or 2 and the number of O being 0 or 1, such as azetidinyl, oxetanyl, unsubstituted or substituted by e.g. C₁₋₄alkyl, such as methyl; 5-membered heterocycloalkyl having 1 or 2 N-atoms, such as pyrrolidinyl, unsubstituted or substituted by e.g. one or more of C₁₋₄alkyl, such as methyl; 6-membered heterocycloalkyl having N and O-atoms, such as morpholinyl, piperazinyl, piperidinyl, dioxanyl, 3-methyl-3-azabicyclo[3.1.0]hexan-1-yl, unsubstituted or substituted by e.g. one or more of C₁₋₄alkyl, such as methyl, halogen, e.g. F; 7-membered heterocycloalkyl having N and O-atoms, such as 2-oxa-5-azabicyclo[2.2.1]heptan-5-yl; 8-membered heterocycloalkyl having N and 0-atoms, such as 8-oxa-3-azabicyclo[3.2.1]octan-3-yl, 1,4-diazabicyclo[3.2.1]octan-4-yl. The term “C₁₋₄ alkyl 4-8 membered heterocycloalkyl” refers to -L²-X¹— or L³-X²— with L², L³ being C1-4 alkyl and X¹, X² being 4-8 membered heterocycloalkyl as defined herein. Thus, the 4-8 membered hetereocycloalkyl is linked through a C₁₋₄ alkyl group as defined to the neighbouring group. In some embodiments, the alkyl may be C₁, resulting in —(CH₂)-(4-8 membered heterocycloalkyl) or C₂, resulting in —(CH₂)₂-(4-8 membered heterocycloalkyl) or C₃, resulting in —(CH₂)₃-(4-8 membered heterocycloalkyl) or C₄, resulting in —(CH₂)₄-(4-8 membered heterocycloalkyl). Examples include —(CH₂)-morpholinyl, —(CH₂)₂-morpholinyl, —(CH₂)₃-morpholinyl, —(CH₂)₄-morpholinyl, —(CH₂)-piperazinyl, —(CH₂)₂—N-methyl-piperazinyl, —(CH₂)₃-piperazinyl or —(CH₂)₄-piperazinyl. The term “—C₁₋₄ alkoxy 4-8 membered heterocycloalkyl” refers to -L²-X¹— or L³-X²— with L², L³ being C₁₋₄ alkoxy and X¹, X² being 4-8 membered heterocycloalkyl as defined herein. Thus, the 4-8 membered hetereocycloalkyl is linked via a C₁₋₄ alkoxy group as defined herein to its neighbouring group. In some embodiments, the C₁₋₄ alkoxy may be C₁, resulting in —(O—CH₂)-(4-8 membered heterocycloalkyl) or C₂, resulting in —(O—CH₂)₂-(4-8 membered heterocycloalkyl) or C₃, resulting in —(O—CH₂)₃-(4-8 membered heterocycloalkyl). Examples include —(O—CH₂)—(N-morpholinyl), —(O—CH₂)₂—(N-morpholinyl). The term “—O-(4-8 membered heterocycloalkyl)” refers to -L²-X¹— or L³-X²— with L², L³ being —O— and X¹, X² being 4-8 membered heterocycloalkyl as defined herein. Thus, the 4-8 membered hetereocycloalkyl is linked through an —O-atom to the neighbouring group. Examples include —O-morpholinyl, —O-piperazinyl, —O-pyrrolidinyl and the like.

The term “halogen” or “hal” as used herein may be fluoro, chloro, bromo or iodo such as fluoro, chloro or bromo, e.g. fluoro or chloro.

The term “C₁₋₄ alkyl” and “C₁₋₆alkyl” refer to a fully saturated branched or unbranched hydrocarbon moiety having 1, 2, 3 or 4 and 1, 2, 3, 4, 5 or 6 carbon atoms, respectively. Representative examples of alkyl include, but are not limited to, methyl, ethyl, n-propyl, iso-propyl, n-butyl, sec-butyl, iso-butyl, tert-butyl, n-pentyl, iso-pentyl, neopentyl, n-hexyl, iso-hexyl or neohexyl.

The term “C₁₋₆ heteroalkyl” refers to an alkyl as defined with 1, 2, 3, 4, 5 or 6 carbon atoms in which at least one carbon atom is replaced with a heteroatom, such as N, O, or S, e.g. N, O. It is understood that the heteroatom may further be substituted with one or two C₁₋₆ alkyl. Examples include —(CH₂)₂—O-Me, —(CH₂)₃—O-Me, —(CH₂)₂—O—CH₂Me, —(CH₂)₂—NMe₂, —(CH₂)—NMe₂, —(CH₂)₂—NEt₂, —(CH₂)—NEt₂ and the like.

The term “C₁₋₄alkylamino” refers to a fully saturated branched or unbranched C₁₋₄ alkyl, which is substituted with at least one, such as only one, amino group, alkylamino group or dialkylamino group, such as NH₂, HN(C₁₋₄alkyl) or N(C₁₋₄alkyl)₂. Thus, a C₁₋₄alkylamino refers to C₁₋₄alkylamino, C₁₋₄alkyl-(C₁₋₄alkyl)amino, C₁₋₄alkyl-(C₁₋₄dialkyl)amino. Examples include but are not limited to methylaminomethyl, dimethylamonimethyl, aminomethyl, dimethylaminoethyl, aminoethyl, methylaminoethyl, n-propylamino, iso-propylamino, n-butylamino, sec-butylamino, iso-butylamino, tert-butylamino.

As used herein and unless otherwise indicated, the term “stereoisomer” refers to “optically pure” or “stereoisomerically pure” compounds used in the present disclosure, which means that the stereoisomer of a compound is substantially free of the other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein, the term “enantiomerically pure” means a stereoisomerically pure composition of a compound having one chiral center.

As used herein, the terms “about” or “approximately” mean an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “readthrough”, as used herein with regard to the process of translation, means (a) reading a premature stop codon or nonsense codon as a codon, which correctly codes for an amino acid (or “sense codon”) or (b) bypassing a premature stop codon.

The term “premature stop codon” or “nonsense codon” or “premature termination codon” (or PTC) as used herein refers to the occurrence of a stop codon (such as UAG, UGA, UAA in mRNA) at a position where a “sense codon”, i.e. a codon correctly coding for an amino acid, should be. The occurrence of such a premature stop codon is typically due to a germline or somatic or sporadic mutation in a DNA (or mRNA) sequence, also referred to herein as a “nonsense mutation” or “premature termination mutation” or “premature stop mutation”, that causes the change of a “sense” codon to a stop codon. In some embodiments, the nonsense mutation is a mutation that occurs in DNA and is then transcribed into mRNA.

Such a (nonsense mutation-mediated) premature termination codon leads to a “premature translation termination”, i.e. an incorrect termination of translation, which prevents the wild-type protein synthesis and leads to the silencing of the mutated gene and no generation of the active, functional protein (total lack) or the generation of a truncated, incomplete and thus non-functional or defective protein (partial lack). This (partial or total) lack of protein may then cause the respective pathology. Currently, it is believed that nonsense mutations that generate PTCs cause at least one-third of all known human genetic diseases (herein also referred to as “nonsense-mutation-mediated (genetic) disease”). For example, a premature termination codon in the gene coding for the dystrophin protein causes nonsense-mutation-mediated-Duchenne Muscular Dystrophy in boys; a premature termination codon in JAKI, SYNJ2 or CLPTM1 genes causes nonsense-mutation-mediated prostatic cancers, etc.

In the process of readthrough as defined above, a suppression (and thus inhibition) of premature translation termination (also referred to as a “nonsense suppression”) or a modulation of premature translation termination takes place and thus allows to restore synthesis of the full length protein. The term “modulation” in the context of premature translation termination refers to the regulation of gene expression by altering the level of nonsense suppression. It refers to an up-regulation of nonsense suppression, if it is desirable to increase production of a functional, active protein, which is otherwise defective as the gene encoding it includes a premature stop codon. This is achieved by permitting readthrough of the premature stop codon in the gene such that translation of the mRNA can occur, and typically requires the use of a nonsense suppression compound. Alternatively, it refers to a down-regulation of nonsense suppression, if it is desirable to promote the degradation of an mRNA with a premature stop codon.

Thus, a compound of formula I, which is capable of a translational readthrough of a premature termination codon (i.e. achieving a nonsense suppression) when used in a monotherapy or in a combined therapy with an aminoglycoside allows to restore functional protein expression and to reduce disease symptoms associated with the production of otherwise (partially) non-functional protein(s).

As used herein, the term “combination” (as in e.g. “in combination” or “a combination of”) refers to the use (e.g. administration) of one or more compounds of the disclosure and one or more aminoglycosides of the disclosure leading to a synergistic therapeutic effect. The term “combination” or “pharmaceutical combination” includes any of the combinations of one or more compounds of the disclosure and one or more aminoglycosides of the disclosure (or any therapy comprising one or more compounds of the disclosure and one or more aminoglycosides of the disclosure), which are suitable for use in the (or methods of) prevention or treatment of a disease or disorder caused by or associated with one or more premature termination codons in a subject, e.g. a human, comprising administering the active agents, pharmaceutical compositions comprising the active agents for simultaneous or sequential use.

A combination or pharmaceutical combination may include both a fixed combination of the active agents in one dosage form, or non-fixed combination of the active agents in separate dosage forms for a combined therapy, wherein the active agents may be administered independently but at the same time, or sequentially without restriction of the order or the timing of administration (i.e. the time interval between administration of a compound of formula I and an aminoglycoside of the disclosure or pharmaceutical compositions thereof).

It is understood that the time intervals are designed such that the combination of active agents leads to a synergistic effect.

The term “monotherapy” refers to the administration of one (or more) compound of the disclosure to treat a disease or disorder defined herein. The term “combined therapy” or “combination therapy” refers to the administration of two (or more) active agents to treat a disease or disorder defined in the present disclosure. Such an administration encompasses co-administration of the active agents in a substantially simultaneous manner, e.g. in a single dosage form having a fixed ratio of specific amounts of active agents or in separate dosage forms (e.g., capsules, intravenous formulations, and the like) for each active agent in specific amounts. Alternatively, such an administration also encompasses co-administration of the active agents in a sequential manner, namely in separate dosage forms (e.g., capsules, intravenous formulations, and the like) for each active agent in specific amounts. The term “fixed” or “single” in reference to a combination of active agents refers to a single carrier or vehicle or dosage form comprising the two or more active agents in specific amounts (and ratio). The term “non-fixed” in reference to a combination of active agents means that the two or more active agents are administered to a patient as separate dosage forms either simultaneously or sequentially in time intervals such that the active agents exert a synergistic therapeutic effect.

As used herein, the term “synergistic” refers to a combination of a compound of formula I, and an aminoglycoside compound as defined herein, which combination is more effective than the additive effects of treatments with or administration of a compound of formula I only or an aminoglycoside only. Thus, a synergistic effect may result in improved efficacy of treatments in the prevention, treatment, management or amelioration of a disorder. A synergistic effect of a combined therapy may also permit the use of lower dosages of one or more of the compounds as defined herein and/or less frequent administration of the treatments to a subject with the disorder. The ability to utilize lower dosages of a treatment and/or to administer the treatment less frequently may have the effect of eliminating or reducing adverse or unwanted side effects, including toxicity, associated with the use of either treatment or administration alone either without reducing the efficacy or improving the efficacy of the treatments in the prevention, treatment, management or amelioration of a disorder. Synergistically effective amounts are the amounts to effect a desired therapeutic or prophylactic effect.

The term “prevention” or “preventing” refers to reducing or eliminating the onset of the symptoms or complications of a disease. In some embodiments, such prevention comprises the step of administering a therapeutically effective amount of a compound of formula I in combination with a therapeutically effective amount of an aminoglycoside compound as disclosed herein (or a pharmaceutical composition or combination of pharmaceutical compositions disclosed herein) to a subject in need thereof (e.g., a mammal, e.g., a human).

The term “treatment” or “treating” is intended to encompass therapy and cure. In some embodiments, such treatment comprises the step of administering a therapeutically effective amount of a compound of formula I in combination with a therapeutically effective amount of an aminoglycoside compound as disclosed herein (or a pharmaceutical composition or combination of pharmaceutical compositions disclosed herein) to a subject in need thereof (e.g., a mammal, e.g., a human). In some embodiments, the term “treating” or “treatment” refers to a therapeutic combination treatment of the disclosure leading to an amelioration of the targeted pathologic condition or disorder. In some embodiments, the term “treating” or “treatment” refers to a therapeutic combination treatment or combined therapy of the disclosure leading to curing the targeted pathologic condition or disorder. Subjects in need of therapeutic combination treatment of the disclosure include subjects having the disorder as well as subjects prone to have the disorder. For example, when treating cancer according to a method of the disclosure, a subject or mammal is successfully “treated” for cancer if, after receiving a therapeutic amount of a compound of formula I and an aminoglycoside as defined herein, the subject shows observable and/or measurable reduction in or absence of (i) a reduction in the number of cancer cells (or absence of cancer cells) and/or (ii) a reduction in the proliferation or survival of cancer cells; and/or (iii) relief of one or more of the symptoms associated with the specific cancer and/or (iv) reduced mortality, and/or (v) improved quality of life. Any of these parameters are readily measurable by routine procedures familiar to a physician.

A “therapeutically effective amount” includes the amount of a compound of formula I and the amount of an aminoglycoside as identified in the present disclosure sufficient to allow readthrough of the premature termination codon(s) causing the pathologic condition or disease. A “therapeutically effective amount” also includes the amount of a compound of formula I and the amount of an aminoglycoside as identified in the present disclosure sufficient to achieve suppression of the premature stop mutations within a gene. The therapeutically effective amounts used in the present combined therapy are the synergistically effective (therapeutic) amounts used to effect treatment due to the synergistic effects of the compound of formula I when administered in combination with an aminoglycoside. Thus, a “therapeutically effective amount” as used herein, refers to the amount of a compound of formula I and/or an aminoglycoside as identified in the present disclosure such that upon administration of a compound of formula I in combination with an aminoglycoside as identified in the present disclosure to a subject in need thereof it is sufficient to effect partial or total treatment of a disorder or disease as defined herein or prevention or recurrence or spread of a disease or disorder as defined herein. For example, a disease or disorder such as cancer (i.e. by eliminating, modifying, or controlling a primary, regional or metastatic cancer cell or tissue; delaying or minimizing spreading of cancer; and thus providing an improvement of treatment and management of cancer).

It is understood that the “therapeutically effective amount” may vary depending on the compound of formula I used and/or the aminoglycoside of the disclosure used, the specific nature of the disease and its status or severity, the age, weight, other medical conditions, route of administration, sequential or simultaneous use of the active agents, etc., of the subject to be treated. The therapeutically effective amount may also vary depending on one or more past or concurrent medical, surgical, or other therapy interventions. The therapeutically effective amount may also vary depending on whether it is use for treatment of a disease or disorder as defined herein or whether it is used prophylactically in the prevention or recurrence or spread of the disease or disorder as defined herein, which includes (but is not limited to) those subjects that are predisposed to the disease (e.g., known to possess a gene(s) having one or more nonsense mutations).

According to the disclosure, the term “subject” (or “patient”) refers to any mammal, including humans, domestic and farm animals, and zoo, sports, or pet animals, such as dogs, cats, cattle, horses, sheep, pigs, goats, rabbits, etc. In some embodiments, the subject is a human. In some embodiments, the subject is a fetus, embryo, infant, child, adolescent or adult. In some embodiments, the subject possesses a nonsense mutation (as determined by suitable screening techniques). In some embodiments, the subject possesses a specific premature stop codon, i.e., UAA or UGA or UAG (as determined by suitable screening techniques.

The term “pharmaceutical composition(s) (of the disclosure)” refers to a composition comprising a compound of formula I or an aminoglycoside of the disclosure or a compound of formula I and an aminoglycoside of the disclosure and at least one pharmaceutically acceptable excipient.

The term “dose(s)” as used herein refers to a predetermined amount of a compound of formula I or an aminoglycoside as defined herein to be administered to a patient at one time. The term “dosage” as used herein refers to the number of doses administered to a patient over a specified period of time, e.g. 1 day, 1 week, which is sufficient to achieve the synergistic, therapeutic effect. The term “(unit) dosage form” as used herein refers to a predetermined amount of a pharmaceutical composition (comprising one or more active agents according to the disclosure, i.e. one or more doses, and at least one pharmaceutically acceptable excipient) formulated as a (unit) dosage form suitable for a specific administration route, e.g. liquid dosage forms suitable for parenteral administration (e.g. iv solutions) or oral administration (e.g. syrups); solid dosage forms, such as tablets, capsules, etc., suitable for oral administration; and the like. The term “unit dosage form” refers to discrete units to be administered to a patient. In a combined therapy according to the invention a unit dosage form may be a single dosage form comprising the active agents, e.g. an iv solution comprising two (or more) of the active agents, or a unit dosage form may be multiple dosage forms each comprising one of the two or more active agents, for example two intravenous solutions each comprising one of two active agents or capsules, of which two capsules comprise each a predetermined amount of one of two active agents and a third capsule comprises a predetermined amount of the second of two active agents.

As used herein, the terms “dosing regimen” or “dosage(s)” as used herein refer to the predetermined amount of a compound and/or an aminoglycoside as defined herein, the frequency and the duration of administration during a given time period to achieve a desired synergistic therapeutic or prophylactic effect.

The present disclosure provides in a first aspect a compound or pharmaceutically acceptable salt or stereoisomer thereof of formula I:

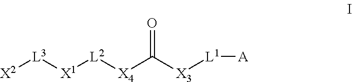

wherein A is

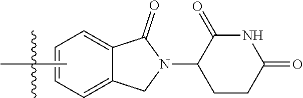

X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆ alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—; X⁴ is —NH—, —CH₂—; L¹ is a covalent bond, C₁₋₆ alkyl, which is unsubstituted or substituted with one or more of C₁₋₄ alkyl, halogen; L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen for use in the prevention and treatment of a disease or disorder caused by or associated with one or more premature termination codons.

Thus, the compound or pharmaceutically acceptable salt or stereoisomer thereof of formula I may be used for enhancing production in a subject of a functional protein from a gene, which is disrupted by the presence of a premature termination codon or the presence of a genetic mutation in the coding region of the gene, or enhancing readthrough of a mRNA premature stop-codon, said mRNA premature stop-codon arising from the translation of a gene comprising a nonsense-mutation.

The compound or pharmaceutically acceptable salt or stereoisomer thereof of formula I may be used in monotherapy for the prevention and treatment of a disease or disorder caused by or associated with one or more premature termination codons.

Alternatively, the compound or pharmaceutically acceptable salt or stereoisomer thereof of formula I may be used in a combined therapy with an aminoglycoside or a pharmaceutically acceptable salt or stereoisomer thereof for the prevention and treatment of a disease or disorder caused by or associated with one or more premature termination codons.

Accordingly, the present disclosure also provides a pharmaceutical combination comprising or consisting of:
(a) a compound of formula I

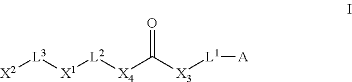

wherein A is

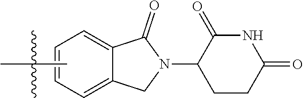

X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆ alkylC(O)OH, —C₁₋₆alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—; X⁴ is —NH—, —CH₂—; L¹ is a covalent bond, C₁₋₆ alkyl, which is unsubstituted or substituted with one or more of C₁₋₄ alkyl, halogen; L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen, and
(b) an aminoglycoside or a pharmaceutically acceptable salt thereof.

The pharmaceutical combination of the disclosure may be used for (i) enhancing production of a functional protein during translation of a mRNA in a subject, which mRNA is disrupted by the presence of a premature termination codon or a genetic mutation in the coding region of the gene, or (ii) enhancing readthrough of a mRNA premature stop-codon, said mRNA premature stop-codon arising from the translation of a gene comprising a nonsense-mutation, and thus may be used in the prevention and treatment of a disease or disorder caused by or associated with one or more premature termination codons. In some embodiments, the premature termination codon is UGA. In some embodiments, the premature termination codon is UAG. In some embodiments, the premature termination codon is UAA.

In some embodiments, the aminoglycoside for use in the combined therapy according to the present disclosure is a compound that typically is characterized by two to three aminosugars linked to form a 2-deoxystreptamine ring by glycosidic linkages. In some embodiments, an aminoglycoside for use in the present disclosure is selected from the group consisting of, but not limited to, geneticin (G418), ELX-02, rhodostreptomycin, streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, paromomycin, lividomycin, apramycin, as well as derivatives of any of these aminoglycosides, including synthetic and semi-synthetic derivatives, (see also, for example, “Aminoglycosides: Molecular Insights on the Recognition of RNA and Aminoglycoside Mimics,” Chittapragada M. et al., Perspectives in Medicinal Chemistry, 2009: 3 21-37). In some embodiments, the aminoglycoside for use in the present disclosure is geneticin (G418) and ELX-02.

In some embodiments of a compound of formula I, —X⁴—CO—X³— is —NH—CO—NH—. In some embodiments of a compound of formula I, —X⁴—CO—X³— is —NH—CO—O—. In some embodiments of a compound of formula I, —X⁴—CO—X³— is —CH₂—CO—NH—. In some embodiments of a compound of formula I, —X⁴—CO—X³— is —CH₂—CO—O—.

In some embodiments of a compound of formula I, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X1 is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, —NH₂, C₁₋₄ alkylhydroxy, or C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, or C₁₋₆ alkoxy.

In some embodiments of a compound of formula I, X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, —C₁₋₄ alkylhydroxy.

In some embodiments of a compound of formula I, L¹ is a linear or branched C₁₋₆ alkyl. In some embodiments of a compound of formula I, L¹ is linear or branched C₁₋₄ alkyl, such as —CH₂— or —CH(CH₃)—.

In some embodiments of a compound of formula I, L² is a covalent bond. In some embodiments of a compound of formula I, L² is linear or branched C₁₋₆ alkyl, such as linear or branched C₁₋₄ alkyl, e.g. —CH₂— or —CH(CH₃)—.

In some embodiments of a compound of formula I, L³ is a covalent bond. In some embodiments L³ is linear or branched C₁₋₄ alkyl. In some embodiments of a compound of formula I, L³ is —O—. In some embodiments of a compound of formula I, L³ is linear or branched C₁₋₄ alkoxy, such as —O—CH₂—, —O—CH₂—CH₂—.

In some embodiments of a compound of formula I, L¹ is —CH₂— and L² is a covalent bond. In some embodiments of a compound of formula I, L¹ is —CH₂— and L² is —CH₂—. In some embodiments of a compound of formula I, L¹ is —CH₂— and L² is —CH(CH₂)—.

In some embodiments of a compound of formula I, L¹ is —CH₂—, L² is a covalent bond and L³ is a covalent bond. In some embodiments of a compound of formula I, L¹ is —CH₂—, L² is a covalent bond and L³ is —CH₂—. In some embodiments of a compound of formula I, L¹ is —CH₂—, L² is a covalent bond and L³ is —O—. In some embodiments of a compound of formula I, L¹ is —CH₂—, L² is a covalent bond and L³ is —O—CH₂—. In some embodiments of a compound of formula I, L¹ is —CH₂—, L² is a covalent bond and L³ is —O—CH₂—CH₂—.

In some embodiments the compound of the disclosure used in the methods of the disclosure is a compound or a pharmaceutically acceptable salt or stereoisomer thereof of formula II or III

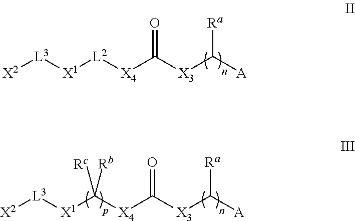

wherein A is

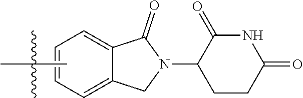

X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆ alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—; X⁴ is —NH—, —CH₂—;
L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen;
R^{a} is a H or C₁₋₄ alkyl; R^{b}, R^{c} are independently of each other H, C₁₋₄ alkyl, such as methyl, ethyl, or halogen, e.g. F;
n is 1, 2; and p is 0, 1, 2.

In some embodiments of a compound of formula II, L² is a covalent bond. In some embodiments of a compound of formula II or III, L² is linear or branched C₁₋₆ alkyl, such as linear or branched C₁₋₄ alkyl, e.g. —CH₂— or —CH(CH₃)—.

In some embodiments of a compound of formula II or III, R^{a} is H. In some embodiments of a compound of formula II or III, R^{a} is linear or branched C₁₋₄ alkyl, such as methyl.

In some embodiments of a compound of formula II or III, n is 1.

In some embodiments of a compound of formula II, R^{a} is H, n is 1 and L² is a covalent bond. In some embodiments of a compound of formula II, R^{a} is H, n is 1 and L² is —CH₂—. In some embodiments of a compound of formula II, R^{a} is H, n is 1 and L² is —CH(CH₂)—.

In some embodiments of a compound of formula II, R^{a} is H, n is 1, L² is a covalent bond and L³ is a covalent bond. In some embodiments of a compound of formula II, R^{a} is H, n is 1, L² is a covalent bond and L³ is —CH₂—. In some embodiments of a compound of formula II, R^{a} is H, n is 1, L² is a covalent bond and L³ is —O—. In some embodiments of a compound of formula II, R^{a} is H, n is 1, L² is a covalent bond and L³ is —O—CH₂—. In some embodiments of a compound of formula II, R^{a} is H, n is 1, L² is a covalent bond and L³ is —O—CH₂—CH₂—.

In some embodiments of a compound of formula III, R^{b} and R^{c} are H. In some embodiments of a compound of formula III, R^{b} is linear or branched C₁₋₄ alkyl, such as methyl and R^{c} is H.

In some embodiments of a compound of formula III, R^{a} is H, n is 1 and p is 0. In some embodiments of a compound of formula II or III, R^{a} is H, n is 1, p is 1.

In some embodiments of a compound of formula II or III, —X⁴—CO—X³— is —NH—CO—NH—. In some embodiments —X⁴—CO—X³— is —NH—CO—O—. In some embodiments of a compound of formula II or III, —X⁴—CO—X³— is —CH₂—CO—NH—. In some embodiments of a compound of formula II or III, —X⁴—CO—X³— is —CH₂—CO—O—.

In some embodiments of a compound of formula II or III, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, —NH₂, C₁₋₄ alkylhydroxy, or C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, or C₁₋₆ alkoxy.

In some embodiments of a compound of formula II or III, X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, —C₁₋₄ alkylhydroxy.

In some embodiments of a compound of formula II or III, L³ is a covalent bond. In some embodiments L³ is linear or branched C₁₋₄ alkyl. In some embodiments of a compound of formula II or III, L³ is —O—. In some embodiments of a compound of formula II or III, L³ is linear or branched C₁₋₄ alkoxy, such as —O—CH₂—, —O—CH₂—CH₂—.

In some embodiments of a compound of formula III, R^{a} is H, n is 1, p is 0 and L³ is a covalent bond. In some embodiments of a compound of formula III, R^{a} is H, n is 1, p is 0 and L³ is —CH₂—. In some embodiments of a compound of formula III, L¹ is —CH₂—, p is 0 and L³ is —O—. In some embodiments of a compound of formula III, L¹ is —CH₂—, p is 0 and L³ is —O—CH₂—. In some embodiments of a compound of formula III, L¹ is —CH₂—, p is 0 and L³ is —O—CH₂—CH₂—.

In some embodiments the compound of the disclosure used in the methods of the disclosure is a compound or a pharmaceutically acceptable salt or stereoisomer thereof of formula IV, V, VI or VII,

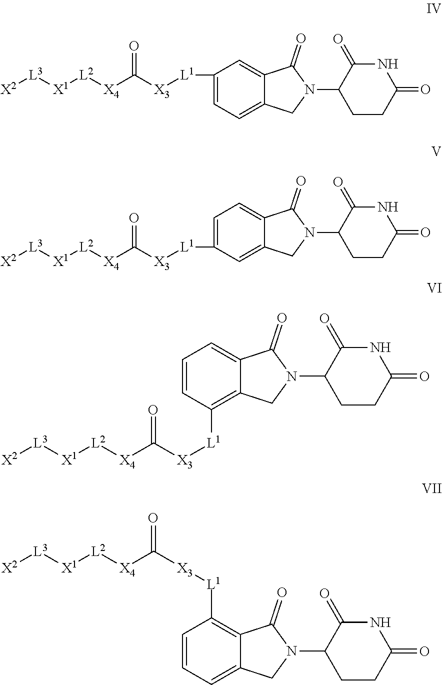

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆ alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—; X⁴ is —NH—, —CH₂—;
L¹ is a covalent bond, C₁₋₆ alkyl, which is unsubstituted or substituted with one or more of C₁₋₄ alkyl, halogen; L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen.

In some embodiments of a compound of formula IV, V, VI or VII, —X⁴—CO—X³— is —NH—CO—NH—. In some embodiments of a compound of formula I, —X⁴—CO—X³— is —NH—CO—O—. In some embodiments of a compound of formula I, —X⁴—CO—X³— is —CH₂—CO—NH—. In some embodiments of a compound of formula I, —X⁴—CO—X³— is —CH₂—CO—O—.

In some embodiments of a compound of formula IV, V, VI or VII, L¹ is a linear or branched C₁₋₆ alkyl. In some embodiments of a compound of formula I, L¹ is linear or branched C₁₋₄ alkyl, such as —CH₂— or —CH(CH₃)—.

In some embodiments of a compound of formula IV, V, VI or VII, L¹ is —CH₂— and L² is a covalent bond. In some embodiments of a compound of formula IV, V, VI or VII, L¹ is —CH₂— and L² is —CH₂—. In some embodiments of a compound of formula IV, V, VI or VII, L¹ is —CH₂— and L² is —CH(CH₂)—.

In some embodiments of a compound of formula IV, V, VI or VII, L¹ is —CH₂—, L² is a covalent bond and L³ is a covalent bond. In some embodiments of a compound of formula IV, V, VI or VII, L¹ is —CH₂—, L² is a covalent bond and L³ is —CH₂—. In some embodiments of a compound of formula IV, V, VI or VII, L¹ is —CH₂—, L² is a covalent bond and L³ is —O—. In some embodiments of a compound of formula IV, V, VI or VII, L¹ is —CH₂—, L² is a covalent bond and L³ is —O—CH₂—. In some embodiments of a compound of formula IV, V, VI or VII, L¹ is —CH₂—, L² is a covalent bond and L³ is —O—CH₂—CH₂—.

In some embodiments the compound of formula I is a compound of formula IVa and IVb, Va and Vb, VIa and VIb, or VIIa and VIIb or a pharmaceutically acceptable salt or stereoisomer thereof,

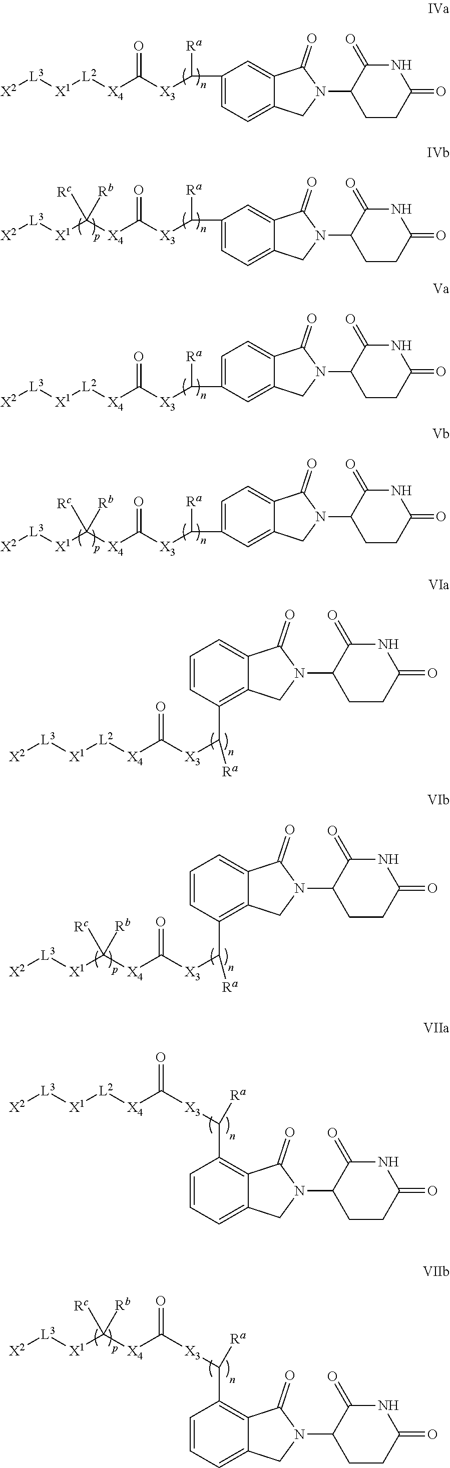

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆ alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—; X⁴ is —NH—, —CH₂—;
L² is a covalent bond, C₁₋₆ alkyl, which is unsubstituted or substituted with one or more of C₁₋₄ alkyl, halogen; L³ is a covalent bond, —O—, —C₁₋₄ alkoxy or C₁₋₆ alkyl, which is unsubstituted or substituted with one or more of C₁₋₄ alkyl, halogen;
R^{a} is a H or C₁₋₄ alkyl; R^{b}, R^{c} are independently of each other H, C₁₋₄ alkyl, such as methyl, ethyl, or halogen, such as F;
n is 0, 1, 2; and p is 0, 1, 2.

In some embodiments of a compound of formula IVa, IVb, Va, Vb, VIa, VIb, VIIa and VIIb, —X⁴—CO—X³— is —NH—CO—NH—. In some embodiments of a compound of formula IVa, IVb, Va, Vb, VIa, VIb, VIIa and VIIb, —X⁴—CO—X³— is —NH—CO—O—. In some embodiments of a compound of formula IVa, IVb, Va, Vb, VIa, VIb, VIIa and VIIb, —X⁴—CO—X³— is —CH₂—CO—NH—. In some embodiments of a compound of formula IVa, IVb, Va, Vb, VIa, VIb, VIIa and VIIb, —X⁴—CO—X³— is —CH₂—CO—O—.

In some embodiments of a compound of formula IV, IVa, IVb, V, Va, Vb, VI, VIa, VIb, VII, VIIa and VIIb, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X1 is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, —NH₂, C₁₋₄ alkylhydroxy, or C₁₋₄ alkoxy; or X¹ together with X⁴ forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, or C₁₋₆ alkoxy.

In some embodiments of a compound of formula IV, IVa, IVb, V, Va, Vb, VI, VIa, VIb, VII, VIIa and VIIb, X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, —C₁₋₄ alkylhydroxy.

In some embodiments of a compound of formula IV, IVa, IVb, V, Va, Vb, VI, VIa, VIb, VII, VIIa and VIIb, n is 1.

In some embodiments of a compound of formula IV, IVa, IVb, V, Va, Vb, VI, VIa, VIb, VII, VIIa and VIIb, R^{a} is H. In some embodiments of a compound of formula IV, IVa, IVb, V, Va, Vb, VI, VIa, VIb, VII, VIIa and VIIb, R^{a} is linear or branched C₁₋₄ alkyl, such as methyl.

In some embodiments of a compound of formula IV, IVa, V, Va, VI, VIa, VII and VIIa, L² is a covalent bond. In some embodiments of a compound of formula IV, IVa, V, Va, VI, VIa, VII and VIIa, L² is linear or branched C₁₋₆ alkyl, such as linear or branched C₁₋₄ alkyl, e.g. —CH₂— or —CH(CH₃)—.

In some embodiments of a compound of formula IV, IVb, V, Vb, VI, VIb, VII and VIIb, p is 0. In some embodiments of a compound of formula IV, IVb, V, Vb, VI, VIb, VII and VIIb, p is 1. In some embodiments of a compound of formula IV, IVb, V, Vb, VI, VIb, VII and VIIb, p is 1 and R^{b} and R^{c} are H. In some embodiments of a compound of formula IV, IVb, V, Vb, VI, VIb, VII and VIIb, p is 1, R^{b} is linear or branched C₁₋₄ alkyl, such as methyl and R^{c} is H. In some embodiments of a compound of formula IV, IVb, V, Vb, VI, VIb, VII and VIIb, p is 1, R^{b} is halogen, such as F, and R^{c} is H.

In some embodiments of a compound of formula IV, IVa, IVb, V, Va, Vb, VI, VIa, VIb, VII, VIIa and VIIb, L³ is a covalent bond. In some embodiments L³ is linear or branched C₁₋₄ alkyl. In some embodiments of a compound of formula I, L³ is —O—. In some embodiments of a compound of formula I, L³ is linear or branched C₁₋₄ alkoxy, such as —O—CH₂—, —O—CH₂— CH₂—.

In some embodiments the compound of the disclosure used in the methods of the disclosure is a compound or a pharmaceutically acceptable salt or stereoisomer thereof of formula VIII

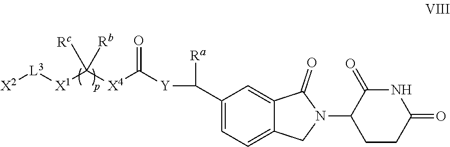

wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆alkyl, —OC(O)—C₁₋₄alkylamino, —C(O)O—C₁₋₆alkyl, —COOH, —CHO, —C₁₋₆ alkylC(O)OH, —C₁₋₆alkylC(O)O—C₁₋₆alkyl, NH₂, C₁₋₆ alkoxy or C₁₋₆ alkylhydroxy; or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
Y is N or O;
R^{a} is a H or C₁₋₄ alkyl; R^{b}, R^{c} are independently of each other H, C₁₋₄ alkyl, such as methyl, ethyl, or halogen, such as F;
L³ is a covalent bond, —O—, —C₁₋₄ alkoxy or C₁₋₆ alkyl, which is unsubstituted or substituted with one or more of C₁₋₄ alkyl, halogen;
p is 0, 1, 2.

In some embodiments of a compound of formula VIII, Y is NH. In some embodiments of a compound of formula VIII, Y is O.

In some embodiments of a compound of formula VIII, R^{a} is H. In some embodiments of a compound of formula VIII, R^{a} is methyl.

In some embodiments of a compound of formula VIII, p is 0. In some embodiments of a compound of formula VIII, p is 1. In some embodiments of a compound of formula VIII, p is 1 and R^{b} and R^{c} are H. In some embodiments of a compound of formula VIII, p is 1, R^{b} is linear or branched C₁₋₄ alkyl, such as methyl and R^{c} is H. In some embodiments of a compound of formula VIII, p is 1, R^{b} is halogen, such as F, and R^{c} is H.

In some embodiments of a compound of formula VIII, L³ is a covalent bond. In some embodiments of a compound of formula VIII, L³ is linear or branched C₁₋₄ alkyl. In some embodiments of a compound of formula VIII, L³ is —O—. In some embodiments of a compound of formula VIII, L³ is linear or branched C₁₋₄ alkoxy, such as —O—CH₂—, —O—CH₂—CH₂—, O—CH₂—CH₂—CH₂—.

In some embodiments of a compound of formula VIII, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, and C₁₋₄ alkylhydroxy, or C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula VIII, X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, p is 0, R^{a} is H and, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 0, R^{a} is H and X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 0, R^{a} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 0, R^{a} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 0, R^{a} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula VIII, p is 0, R^{a} is H and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, p is 0, R^{a} is H and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, p is 0, R^{a} is H and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, p is 0, R^{a} is methyl and, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 0, R^{a} is methyl and X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 0, R^{a} is methyl and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 0, R^{a} is methyl and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 0, R^{a} is methyl and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula VIII, p is 0, R^{a} is methyl and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, p is 0, R^{a} is methyl and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, p is 0, R^{a} is methyl and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, p is 1, R^{b} and R^{c} are H and, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 1, R^{b} and R^{c} are H and X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 1, R^{b} and R^{c} are H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 1, R^{b} and R^{c} are H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 1, R^{b} and R^{c} are H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula VIII, p is 1, R^{b} and R^{c} are H and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, p is 1, R^{b} and R^{c} are H and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, p is 1, R^{b} and R^{c} are H and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, p is 1, R^{b} is methyl and R^{c} is H and, X¹ is linear or branched —C₁₋₆ alkyl, —C₃₋₆ cycloalkyl, —C₆₋₁₀ aryl, 5-10 membered heteroaryl, 5-6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 1, R^{b} is methyl and R^{c} is H and X¹ is linear or branched —C₁₋₆ alkyl, —C₃₋₆ cycloalkyl, —C₆₋₁₀ aryl, 5-10 membered heteroaryl, 6 membered heterocycloalkyl, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 1, R^{b} is methyl and R^{c} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a 4-8 membered heterocycloalkyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 1, R^{b} is methyl and R^{c} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, —O—(CH₂)₂—OMe, OCF₃, OCHF₂, C₁₋₆ alkylamino, —CN, NH₂, C₁₋₄ alkylhydroxy, and C₁₋₆ alkoxy.

In some embodiments of a compound of formula VIII, p is 1, R^{b} is methyl and R^{c} is H and X¹ is linear or branched —C₁₋₆ alkyl, cyclopropyl, cyclobutyl, cyclopentyl, cyclohexyl, piperidinyl, phenyl, pyridinyl, pyrazinyl, pyridazinyl, pyrimidinyl, 2,2-difluoro-1,3-benzodioxolyl, 2,3-dihydrobenzofuryl, 2-methyl-2,3-dihydrobenzofuryl, 3-methyl-2,3-dihydrobenzofuryl, 3,3-dimethyl-2,3-dihydrobenzofuryl, 2,3-dimethyl-2,3-dihydrobenzofuryl, cyclopentenopyridine, benzodihydropyrane, dihydropyrano-pyridine, wherein X¹ is unsubstituted or substituted with one or more of halogen, linear or branched —C₁₋₄ alkyl, CF₃, CHF₂, CMeF₂, —OCF₃, OCHF₂, C₁₋₄ alkylhydroxy, and C₁₋₄ alkoxy; or X¹ together with the N atom of the carbamate forms a pyrrolidinyl, piperdinyl, morpholinyl, piperazinyl, N-methyl piperazinyl, which is unsubstituted or substituted with one or more of halogen, linear or branched —C₁₋₄ alkyl, CF₃, CHF₂, CMeF₂, OCF₃, OCHF₂, C₁₋₄ alkylhydroxy, and C₁₋₆ alkoxy.

In some embodiments of a compound of formula VIII, p is 1, R^{b} is methyl and R is H and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl,
C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, p is 1, R^{b} is methyl and R^{c} is H and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, p is 1, R^{b} is methyl and R^{c} is H and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, L³ is a covalent bond and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is a covalent bond and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is a covalent bond and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, L³ is a covalent bond and X² is cyclopropyl, azetidinyl, oxetanyl, cyclobutyl, pyrrolidinyl, piperdinyl, piperazinyl, morpholinyl, 8-oxa-3-azabicyclo[3.2.1]octan-3-yl, pyridyl, wherein X² is unsubstituted or substituted with one or more of linear or branched C₁₋₆ alkyl, such as methyl, —C₁₋₄ alkoxy, such as methoxy, NH₂, NMe₂ and halogen, such as fluoro.

In some embodiments of a compound of formula VIII, L³ is linear or branched C₁₋₄ alkyl, such as —CH₂—, and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is linear or branched C₁₋₄ alkyl, such as —CH₂—, and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is linear or branched C₁₋₄ alkyl, such as —CH₂—, and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, L³ is linear or branched C₁₋₄ alkyl, such as —CH₂—, and X² is morpholinyl, 2-oxa-5-azabicyclo[2.2.1]heptanyl, 8-oxa-3-azabicyclo[3.2.1]octan-3-yl.

In some embodiments of a compound of formula VIII, L³ is —O— and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is —O— and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is —O— and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, L³ is —O— and X² is cyclopropyl, pyrrolidinyl, N-methyl-pyrrolidinyl,

In some embodiments of a compound of formula VIII, L³ is —O—CH₂— and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is —O—CH₂— and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is —O—CH₂— and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, L³ is —O—CH₂— and X² is pyrrolidinyl, N-methyl-pyrrolidinyl.

In some embodiments of a compound of formula VIII, L³ is —O—CH₂—CH₂— and X² is H, C₃₋₆ cycloalkyl, C₆ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is —O—CH₂—CH₂— and X² is H, C₃₋₆ cycloalkyl, C₆ aryl, 5-6 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy.

In some embodiments of a compound of formula VIII, L³ is —O—CH₂—CH₂— and X² is H, cyclopropyl, cyclobutyl, C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, oxetanyl, methyl-oxetanyl, furanyl, piperazinyl, N-methyl-piperazinyl, azetidinyl, methyl-azetidinyl, N-dimethyl-azetidinyl, 2-oxa-5-azabicyclo[2.2.1]heptanyl, 1,4-diazabicyclo[3.2.1]octan-4-yl, 3-methyl-3-azabicyclo[3.1.0]hexan-1-yl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂ and halogen.

In some embodiments of a compound of formula VIII, L³ is —O—CH₂—CH₂— and X² is morpholinyl.

In some embodiments of a compound of formula VIII, X¹ is a C₆ aryl or 6-membered heteroaryl, such as a pyridine, pyridazine, pyrimidine or pyrazine. In some embodiments of a compound of formula I, X¹ is a partially aromatic 6 to 10 membered heteroaryl, such as a 5-6 or 6-6 fused ring system with a 6 membered ring being a phenyl or pyridyl group. In some embodiments of a compound of formula I, X¹ is a C₁₋₆ alkyl, C₃₋₆ cycloalkyl.

In some embodiments, the present disclosure is directed towards a compound or a pharmaceutically acceptable salt or stereoisomer thereof of formula IXa, IXb, or IXc

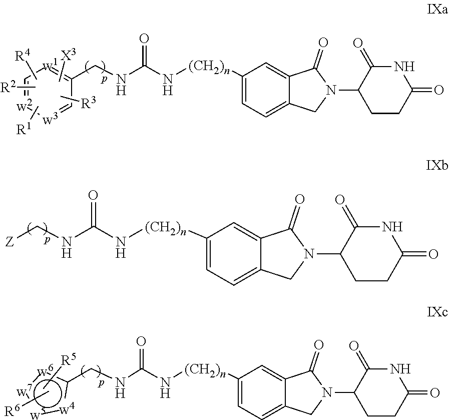

wherein
one of w¹, w² or w³ is selected from C and N, and the other two of w¹, w² or w³ are C;
one or two of w⁴, w⁵, w⁶, w⁷ is selected from C, O, N, NMe, NH, or S while two or three of w⁴, w⁵, w⁶ and w⁷ are C;
X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄ alkyl-4-8 membered heterocycloalkyl, wherein X³ is unsubstituted or substituted with one or more of linear or branched C₁₋₆ alkyl, NH₂, NMe₂ or 5-6 membered heterocycloalkyl;
Z is linear or branched C₁₋₆ alkyl or C₃₋₆ cycloalkyl, C₁₋₄ alkoxy or C₁₋₄ alkyl-C₁₋₄ alkoxy, wherein Z is unsubstituted or substituted with C₁₋₄ alkyl
R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂ —O—(CH₂)₂—OMe, OCF₃, —CN, —N(H)C(O)—C₁₋₆alkyl, —OC(O)—C₁₋₄alkylamino, —OC(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl;
R⁵, R⁶ each are independently selected from hydrogen, linear or branched C₁₋₄ alkyl, CF₃, CHF₂, halogen, such as F, Cl, Br, e.g. F or Cl; n is 1 or 2, and p is 0, 1 or 2.

In some embodiments of a compound of formula IXa, IXb, or IXc, n is 1. In some embodiments of a compound of formula IIIa, IIIb or IIIc, p is 0. In some embodiments of a compound of formula IIIa, IIIb or IIIc, p is 1. In some embodiments of a compound of formula IIIa, IIIb or IIIc, n is 1 and p is 0 or 1. In some embodiments of a compound of formula IIIa, IIIb or IIIc, n is 1 and p is 0.

In some embodiments of a compound of formula IXa, R¹, R², R³ are defined as above and R⁴ is hydrogen such that the aromatic ring contains 4 or 5 substituents which are not hydrogen.

In some embodiments of a compound of formula IXa, R¹ and R² are defined as above and R³ and R⁴ each are hydrogen, such that the aromatic ring contains 3 or 4 substituents which are not hydrogen.

In some embodiments of a compound of formula IXa, IXb, or IXc, R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, n is 1 and R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, such as F, Cl, Br, e.g. F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, n is 1 and R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, p is 0, 1 or 2 and R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, such as F, Cl, Br, e.g. F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, p is 0, 1 or 2 and R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, p is 0 or 1 and R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, such as F, Cl, Br, e.g. F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, p is 0 or 1 and R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, n is 1, p is 0, 1 or 2 and R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂ —O—(CH₂)₂—OMe, OCF₃, —CN, —N(H)C(O)—C₁₋₆alkyl, —OC(O)—C₁₋₄alkylamino, —OC(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, such as F, Cl, Br, e.g. F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, n is 1, p is 0, 1 or 2 and R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, n is 1, p is 0 or 1 and R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂ —O—(CH₂)₂—OMe, OCF₃, —CN, —N(H)C(O)—C₁₋₆alkyl, —OC(O)—C₁₋₄alkylamino, —OC(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl, CF₃, CHF₂, halogen, such as F, Cl, Br, e.g. F or Cl; and X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa, IXb, or IXc, n is 1, p is 0 or 1 and R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; R⁵, R⁶ each are independently selected from hydrogen, linear or branched C₁₋₄ alkyl and CF₃; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXb, C₁₋₆ alkyl is selected from methyl, ethyl, n-propyl, iso-propyl, n-butyl, iso-butyl, t-butyl, n-pentyl, iso-pentyl, neopentyl, n-hexyl, iso-hexyl, and neohexyl.

In some embodiments of a compound of formula IXb, C₃₋₆ cycloalkyl is selected from cyclopropyl, cyclobutyl, cyclopentyl, and cyclohexyl.

In some embodiments of a compound of formula IXb, C₁₋₄ alkoxy is selected from methoxy, ethoxy, n-propoxy, iso-propoxy, n-butoxy, iso-butoxy, and t-butoxy.

In some embodiments of a compound of formula IXb, “C₁₋₄ alkyl-C₁₋₄ alkoxy” is selected from methyl-methoxy, methyl-ethoxy, methyl-n-propoxy, methyl-iso-propoxy, methyl-n-butoxy, methyl-iso-butoxy, methyl-t-butoxy, ethyl-methoxy, ethyl-ethoxy, ethyl-n-propoxy, ethyl-iso-propoxy, ethyl-n-butoxy, ethyl-iso-butoxy, ethyl-t-butoxy, propyl-methoxy, propyl-ethoxy, propyl-n-propoxy, propyl-iso-propoxy, propyl-n-butoxy, propyl-iso-butoxy, and propyl-t-butoxy.

In some embodiments of a compound of formula IXb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl.

In some embodiments of a compound of formula IXb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy.

In some embodiments of a compound of formula IXb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and n is 1.

In some embodiments of a compound of formula IXb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy and n is 1.

In some embodiments of a compound of formula IXb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 0, 1 or 2.

In some embodiments of a compound of formula IXb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 0 or 1.

In some embodiments of a compound of formula IXb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and n is 1 and p is 0, 1 or 2.

In some embodiments of a compound of formula IXb, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and n is 1 and p is 0 or 1.

In some embodiments, the present disclosure is directed to compounds or a pharmaceutically acceptable salt or stereoisomer thereof of formula IXa-1

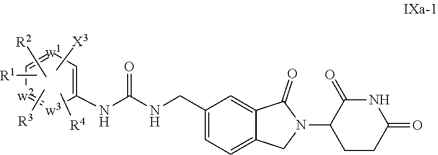

wherein
one of w¹, w² or w³ is selected from C and N, and the other two of w¹, w² or w³ are C;
R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂ —O—(CH₂)₂—OMe, OCF₃, —CN, —N(H)C(O)—C₁₋₆alkyl, —OC(O)—C₁₋₄alkylamino, —OC(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl;
X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄ alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa-1, R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa-1, R¹, R², R³ are defined as above and R⁴ is hydrogen such that the aromatic ring contains 4 or 5 substituents which are not hydrogen.

In some embodiments of a compound of formula IXa-1, R¹ and R² are defined as above and R³ and R⁴ each are hydrogen, such that the aromatic ring contains 3 or 4 substituents which are not hydrogen.

In some embodiments, the present disclosure is directed to compounds or a pharmaceutically acceptable salt or stereoisomer thereof of formula IXa-2, IXa-3, IXa-4 or IXa-5

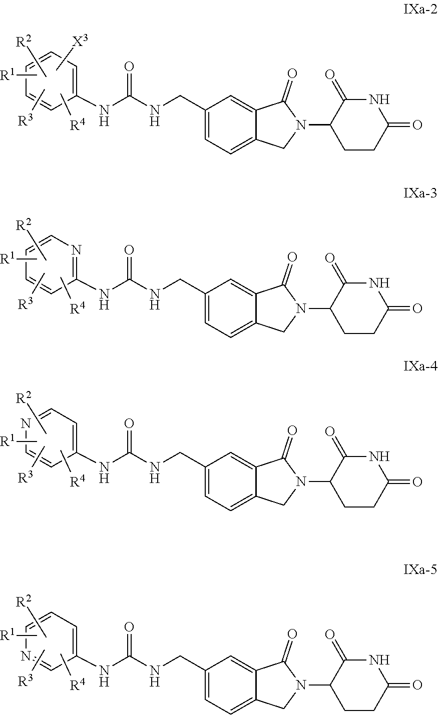

wherein
R¹, R², R³, R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂ —O—(CH₂)₂—OMe, OCF₃, —CN, —N(H)C(O)—C₁₋₆alkyl, —OC(O)—C₁₋₄alkylamino, —OC(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl;
X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁-4 alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa-2, IXa-3, IXa-4 or IXa-5 R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl.

In some embodiments of a compound of formula IXa-2, R¹, R², R³ and R⁴ each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂, OCF₃, —CN, —CHO, —C₁₋₆alkylC(O)OH, NH₂, C₁₋₄ alkylhydroxy, halogen, such as F, Cl, Br, e.g. F or Cl; and/or two of R¹, R², R³, R⁴ form together a 5-6 membered heterocycloalkyl or a 5-6 membered heteroaryl; X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁₋₄alkyl-4-8 membered heterocycloalkyl, wherein X³ is unsubstituted or substituted with one or more of linear or branched C₁₋₆ alkyl, NH₂, NMe₂ or 5-6 membered heterocycloalkyl; while in compounds of formula IIIa-3, IIIa-4 or IIIa-5 R¹ and R² each are independently selected from hydrogen, linear or branched C₁₋₆ alkyl, and halogen, such as Cl, while R³ and R⁴ are hydrogen.

In some embodiments the compound of formula IXb has formula IXb-1

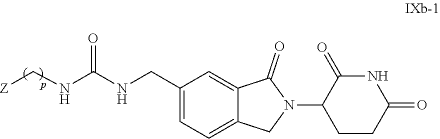

wherein p is 0, 1 or 2; and
Z is linear or branched C₁₋₆ alkyl or C₃₋₆ cycloalkyl, C₁₋₄ alkoxy or C₁₋₄ alkyl-C₁₋₄ alkoxy, wherein Z is unsubstituted or substituted with C₁₋₄ alkyl.

In some embodiments of the compound of formula IXb-1, C₁₋₆ alkyl is selected from methyl, ethyl, n-propyl, iso-propyl, n-butyl, iso-butyl, t-butyl, n-pentyl, iso-pentyl, neopentyl, n-hexyl, iso-hexyl, and neohexyl.

In some embodiments of the compound of formula IXb-1, C₃₋₆ cycloalkyl is selected from cyclopropyl, cyclobutyl, cyclopentyl, and cyclohexyl.

In some embodiments of the compound of formula IXb-1, C₁₋₄ alkoxy is selected from methoxy, ethoxy, n-propoxy, iso-propoxy, n-butoxy, iso-butoxy, and t-butoxy.

In some embodiments of the compound of formula IXb-1, C₁₋₄ alkyl-C₁₋₄ alkoxy” is selected from methyl-methoxy, methyl-ethoxy, methyl-n-propoxy, methyl-iso-propoxy, methyl-n-butoxy, methyl-iso-butoxy, methyl-t-butoxy, ethyl-methoxy, ethyl-ethoxy, ethyl-n-propoxy, ethyl-iso-propoxy, ethyl-n-butoxy, ethyl-iso-butoxy, ethyl-t-butoxy, propyl-methoxy, propyl-ethoxy, propyl-n-propoxy, propyl-iso-propoxy, propyl-n-butoxy, propyl-iso-butoxy, and propyl-t-butoxy.

In some embodiments of the compound of formula IXb-1, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 0.

In some embodiments of the compound of formula IXb-1, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 1.

In some embodiments of the compound of formula IXb-1, Z is methyl, ethyl, n-propyl, iso-propyl, n-butyl, iso-butyl, t-butyl, n-pentyl, iso-pentyl, neopentyl, n-hexyl, iso-hexyl, neohexyl or cyclopropyl, cyclobutyl, cyclopentyl, cyclohexyl, methoxy, ethoxy, n-propoxy, iso-propoxy, n-butoxy, iso-butoxy, t-butoxy, wherein Z is unsubstituted or substituted with methyl, ethyl, n-propyl, iso-propyl, n-butyl, iso-butyl, t-butyl and p is 2.

In some embodiments, the compounds of formula IXc are of formula IXc-1

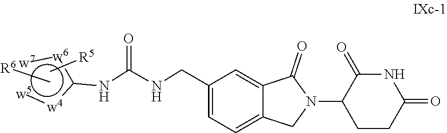

wherein one or two of w⁴, w⁵, w⁶, w⁷ is selected from C, O, N, NMe, NH, or S while two or three of w⁴, w⁵, w⁶ and w⁷ are C;
R⁵, R⁶ each are independently selected from hydrogen, linear or branched C₁₋₄ alkyl, CF₃, CHF₂, halogen, such as F, Cl, Br, e.g. F or Cl.

In some embodiments of compounds of formula IXc-1 or a pharmaceutically acceptable salt or stereoisomer thereof, R⁵, R⁶ each are independently selected from hydrogen, methyl, ethyl and CF₃.

In some embodiments of compounds of formula IXc-1 or a pharmaceutically acceptable salt or stereoisomer thereof, w⁵ is N, w⁷ is NMe, w⁶ and w⁴ are C; or w⁵ is C, w⁷ is S, w⁶ and w⁴ are C; or w⁵ is C, w⁷ is NMe, w⁶ is N and w⁴ is C; or w⁵ is C, w⁷ is C, w⁶ is C and w⁴ is S; or w⁵ is C, w⁷ is C, w⁶ is N and w⁴ is N; or w⁵ is O, w⁷ is C, w⁶ is C and w⁴ is S; or w⁵ is NH, w⁷ is C, w⁶ is C and w⁴ is C; or w⁵ is C, w⁷ is S, w⁶ is C and w⁴ is N; or w⁵ is NH, w⁷ is C, w⁶ is C and w⁴ is N; or w⁵ is C, w⁷ is N, w⁶ is C and w⁴ is S; or w⁵ is NH, w⁷ is N, w⁶ is C and w⁴ is C; or w⁵ is C, w⁷ is NMe, w⁶ is C and w⁴ is C; or w⁵ is N, w⁷ is C, w⁶ is C and w⁴ is S; or w⁵ is C, w⁷ is C, w⁶ is S and w⁴ is N.

In some embodiments of compounds of formula IXc-1 or a pharmaceutically acceptable salt or stereoisomer thereof, R⁵, R⁶ each are independently selected from hydrogen, methyl, ethyl and CF₃ and w⁵ is N, w⁷ is NMe, w⁶ and w⁴ are C; or w⁵ is C, w⁷ is S, w⁶ and w⁴ are C; or w⁵ is C, w⁷ is NMe, w⁶ is N and w⁴ is C; or w⁵ is C, w⁷ is C, w⁶ is C and w⁴ is S; or w⁵ is C, w⁷ is C, w⁶ is N and w⁴ is N; or w⁵ is O, w⁷ is C, w⁶ is C and w⁴ is S; or w⁵ is NH, w⁷ is C, w⁶ is C and w⁴ is C; or w⁵ is C, w⁷ is S, w⁶ is C and w⁴ is N; or w⁵ is NH, w⁷ is C, w⁶ is C and w⁴ is N; or w⁵ is C, w⁷ is N, w⁶ is C and w⁴ is S; or w⁵ is NH, w⁷ is N, w⁶ is C and w⁴ is C; or w⁵ is C, w⁷ is NMe, w⁶ is C and w⁴ is C; or w⁵ is N, w⁷ is C, w⁶ is C and w⁴ is S; or w⁵ is C, w⁷ is C, w⁶ is S and w⁴ is N.

In some embodiments, the present disclosure is directed towards a compound or a pharmaceutically acceptable salt or stereoisomer thereof of formula X, such as Xa, Xb, Xc or Xd

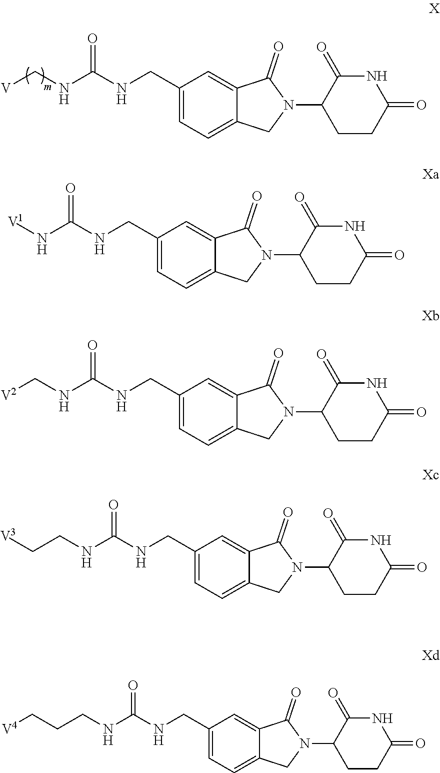

wherein m is 0, 1, 2 or 3, and V, V¹, V², V³, V⁴ is selected from

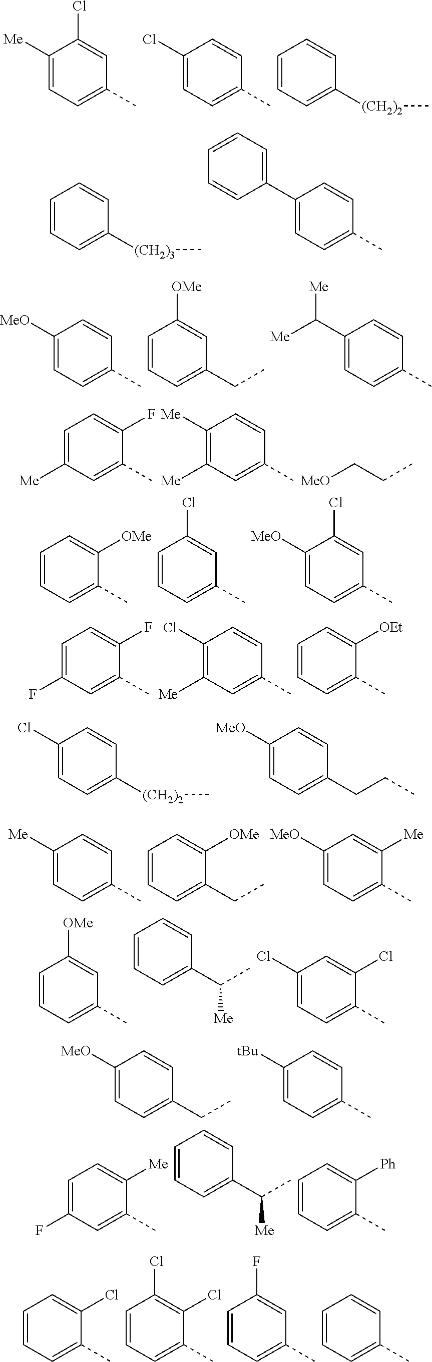

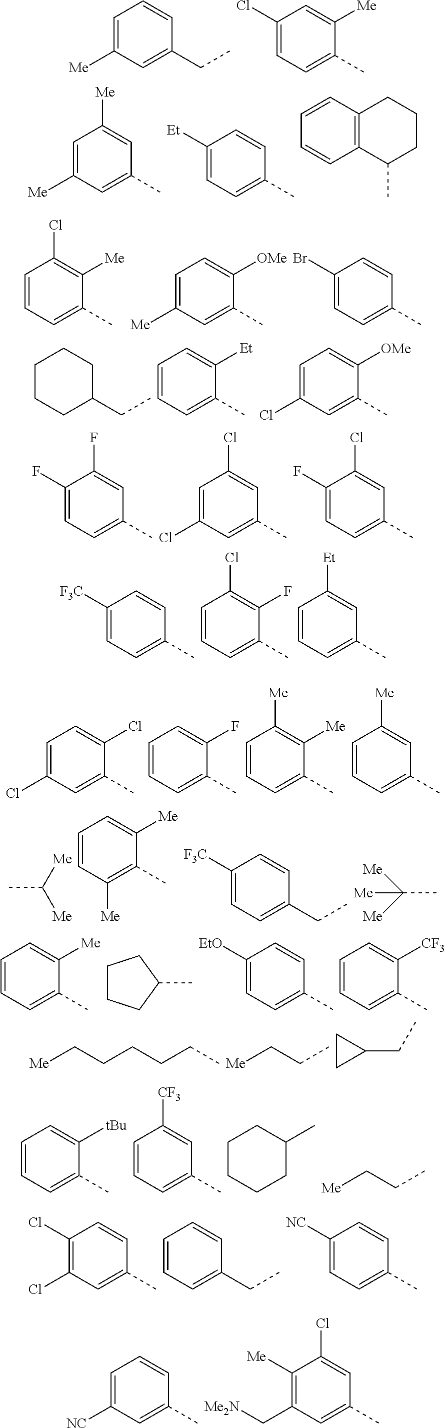

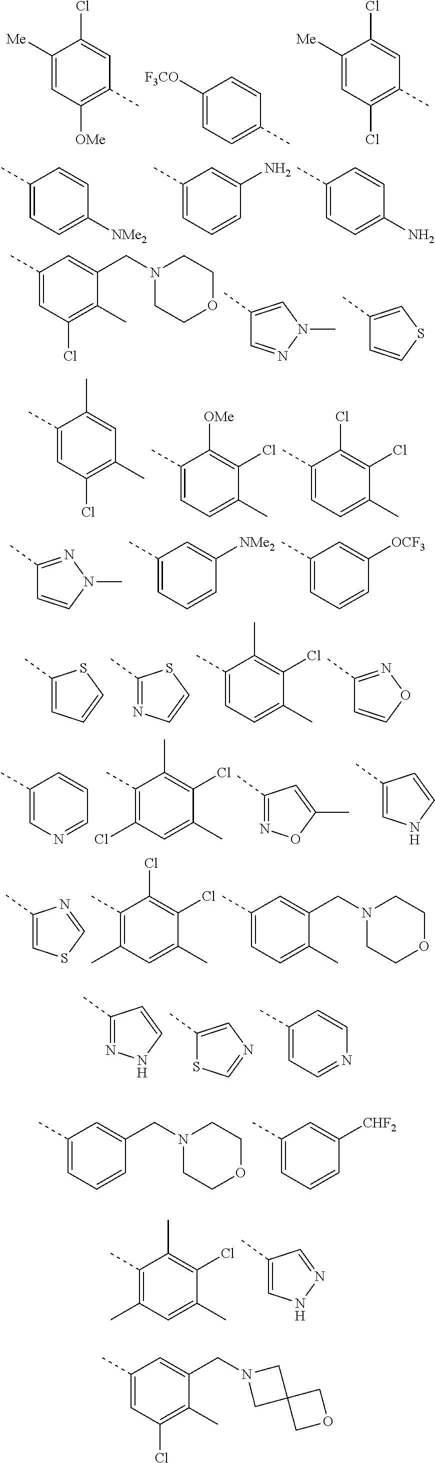

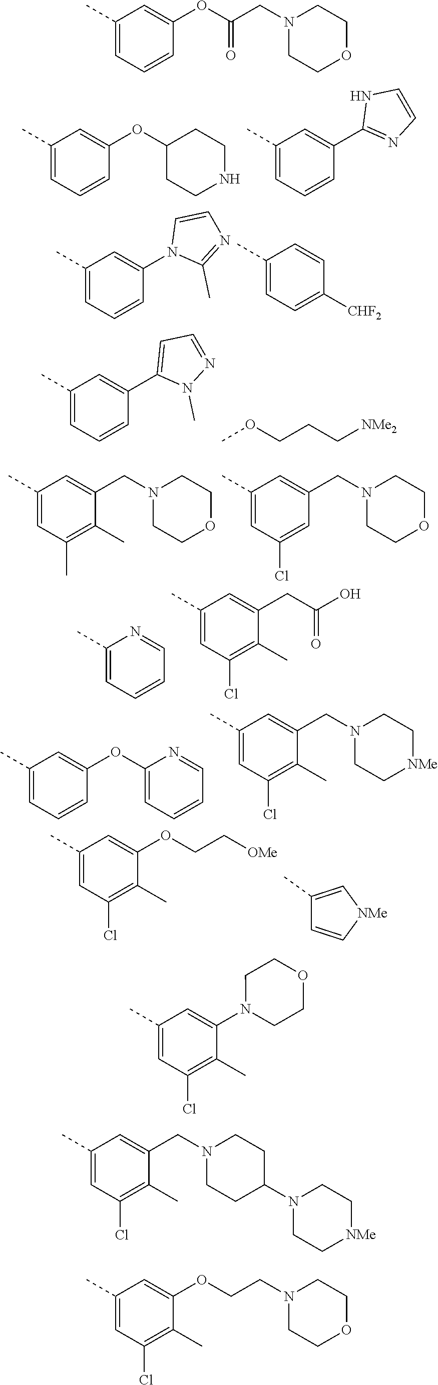

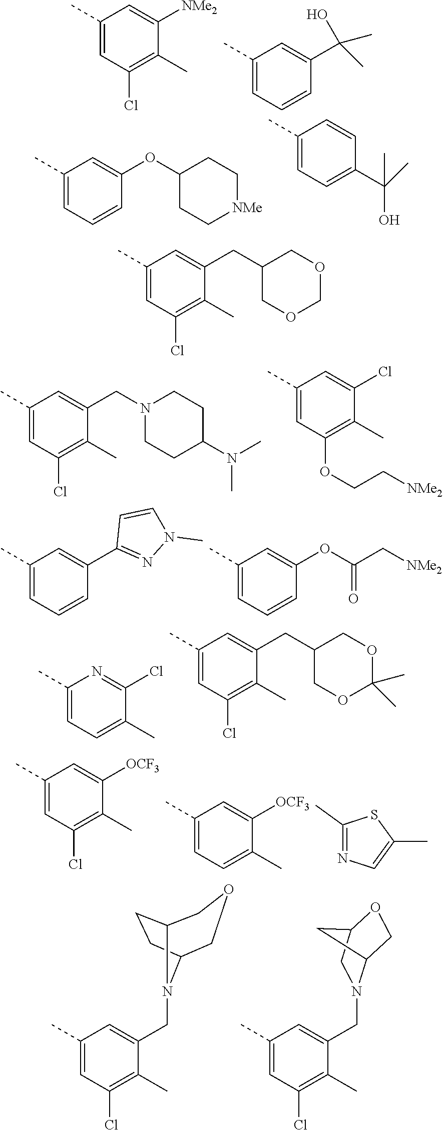

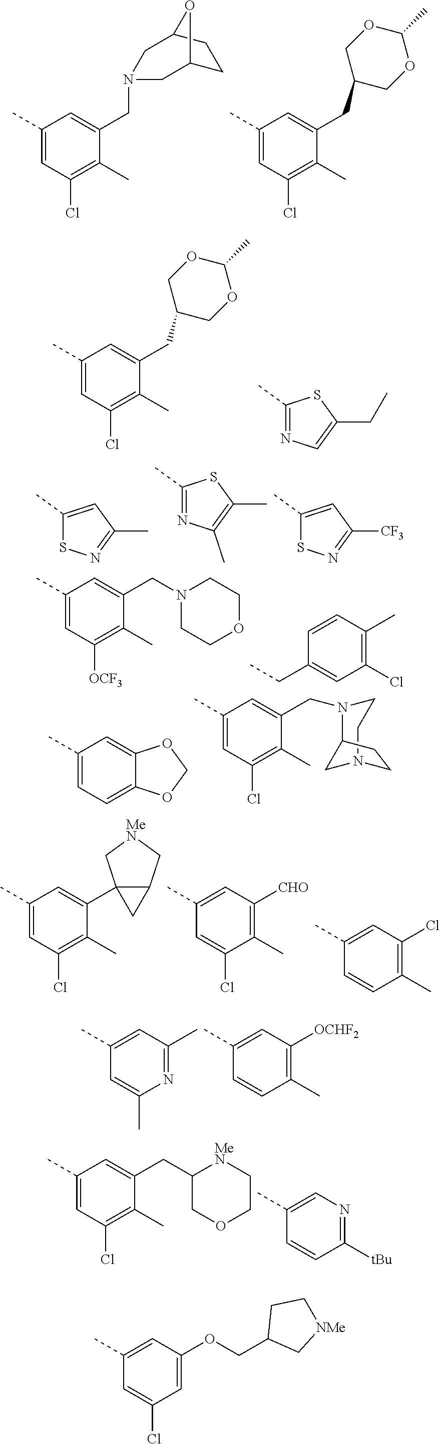

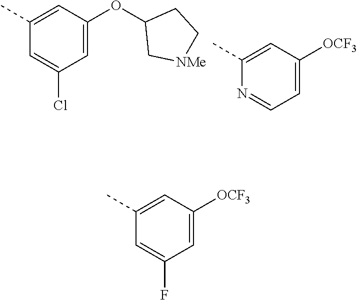

In some embodiments the present disclosure is directed towards a compound or a pharmaceutically acceptable salt or stereoisomer thereof of formula XIa:

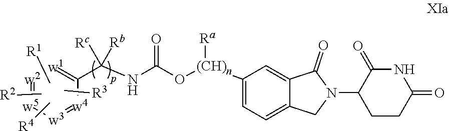

wherein w¹, w², w³, w⁴, w⁵ are independently of each other selected from C and N, with the proviso that at least three of w¹, w², w³, w⁴ w⁵ are C;
R¹, R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R^{a} is H, linear or branched C₁₋₄ alkyl; R^{b}, R^{c} are independently of each other H, linear or branched C₁₋₄ alkyl; n is 1, or 2; and p is 0, 1, or 2.

In some embodiments of a compound of formula XIa, n is 1. In some embodiments n is 1 and R^{a} is H. In some embodiments of a compound of formula XIa, n is 1 and R^{a} is methyl. In some embodiments of a compound of formula XIa, n is 1, p is 0 and R^{a} is H. In some embodiments of a compound of formula XIa, n is 1, p is 0 and R^{a} is methyl.

In some embodiments of a compound of formula XIa, p is 0. In some embodiments of a compound of formula XIa, p is 1. In some embodiments of a compound of formula Va, p is 1, and R^{b} and R^{c} are H. In some embodiments of a compound of formula XIa, p is 1, R^{b} is methyl and R^{c} is H.

In some embodiments of a compound of formula XIa, w¹, w², w³, w⁴, w⁵ are C. In some embodiments of a compound of formula XIa, either w¹ or w² or w³ or w⁴ or w⁵ is N and the remaining 4 of w¹, w², w³, w⁴, w⁵ are C. In some embodiments of a compound of formula XIa, w¹, w² or w¹, w³ or w¹, w⁴ or w², w³ are N and the remaining 3 of w¹, w², w³, w⁴, w⁵ are C. In some embodiments of a compound of formula XIa, w¹, w², w³, w⁴, w⁵ are C.

In some embodiments of a compound of formula XIa, L³ is a covalent bond. In some embodiments of a compound of formula XIa, L³ is linear or branched C₁₋₄ alkyl, such as —CH₂—. In some embodiments of a compound of formula XIa, L³ is —O—. In some embodiments of a compound of formula XIa, L³ is linear or branched C₁₋₄ alkoxy, such as —O—CH₂—, —O—(CH₂)₂—.

In some embodiments of a compound of formula XIa, R¹ is selected from linear or branched —C₁₋₆ alkyl, —C₁₋₆ alkoxy, CF₃, CHF₂, CMeF₂, OCF₃, OCHF₂, CN, and halogen, e.g. F or Cl, and R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa, R¹ is a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa, R¹ is C₃₋₆ cycloalkyl, C₆₋₁₀ aryl, C₆₋₁₀ aryloxy, 5-10 membered heteroaryl, 4-8 membered heterocycloalkyl, wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa, R¹ is —C₁₋₄ alkyl-C₃₋₆ cycloalkyl, —C₁₋₄ alkyl-C₆₋₁₀ aryl, —C₁₋₄ alkyl-(5-10 membered heteroaryl), —C₁₋₄ alkyl-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa, R¹ is —O—C₃₋₆ cycloalkyl, —O—C₆₋₁₀ aryl, —O-(5-10 membered heteroaryl), —O-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa, R¹ is —C₁₋₄ alkoxy-C₃₋₆ cycloalkyl, —C₁₋₄ alkoxy-C₆₋₁₀ aryl, —C₁₋₄ alkoxy-(5-10 membered heteroaryl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is H; and p is 0.

In some embodiments of a compound of formula XIa, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is H; and p is 0.

In some embodiments of a compound of formula XIa, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆ alkyl, NH₂, —C₁₋₆ alkylhydroxy, and halogen, such as F, Cl or Br, e.g. F or Cl; and n is 1, R^{a} is H; and p is 0.

In some embodiments of a compound of formula XIa, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is H; and p is 0.

In some embodiments of a compound of formula XIa, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is H; and p is 0.

In some embodiments of a compound of formula XIa, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is CH₃; and p is 0.

In some embodiments of a compound of formula XIa, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is CH₃; and p is 0.

In some embodiments of a compound of formula XIa, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆ alkyl, NH₂, —C₁₋₆ alkylhydroxy, and halogen, such as F, Cl or Br, e.g. F or Cl; and n is 1, R^{a} is CH₃; and p is 0.

In some embodiments of a compound of formula XIa, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is CH₃; and p is 0.

In some embodiments of a compound of formula XIa, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is CH₃; and p is 0.

In some embodiments of a compound of formula XIa, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{b}, R^{c} are H.

In some embodiments of a compound of formula XIa, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{b}, R^{c} are H.

In some embodiments of a compound of formula XIa, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆ alkyl, NH₂, —C₁₋₆ alkylhydroxy, and halogen, such as F, Cl or Br, e.g. F or Cl; and n, p are 1, R^{a}, R^{b}, R^{c} are H.

In some embodiments of a compound of formula XIa, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n, p are 1, R^{a}, R^{b}, R^{c} are H.

In some embodiments of a compound of formula XIa, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n, p are 1, R^{a}, R^{b}, R^{c} are H.

In some embodiments of a compound of formula XIa, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{c} are H, R^{b} is CH₃.

In some embodiments of a compound of formula XIa, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{c} are H, R^{b} is CH₃.

In some embodiments of a compound of formula XIa, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆ alkyl, NH₂, —C₁₋₆ alkylhydroxy, and halogen, such as F, Cl or Br, e.g. F or Cl; and n, p are 1, R^{a}, R^{c} are H, R^{b} is CH₃.

In some embodiments of a compound of formula XIa, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n, p are 1, R^{a}, R^{c} are H, R^{b} is CH₃.

In some embodiments of a compound of formula XIa, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n, p are 1, R^{a}, R^{c} are H, R^{b} is CH₃.

Some embodiments of the compound of formula XIa are provided by formula XIa-1, wherein w¹ to w⁵ are C, and by formula XIa-2, XIa-3, and XIa-4, wherein one of w¹ to w⁵ is N, for example, wherein w¹ is N, w² to w⁵ are C; or w² is N, w¹ and w³ to w⁵ are C; or w³ is N, w¹, w² and w⁴, w⁵ are C

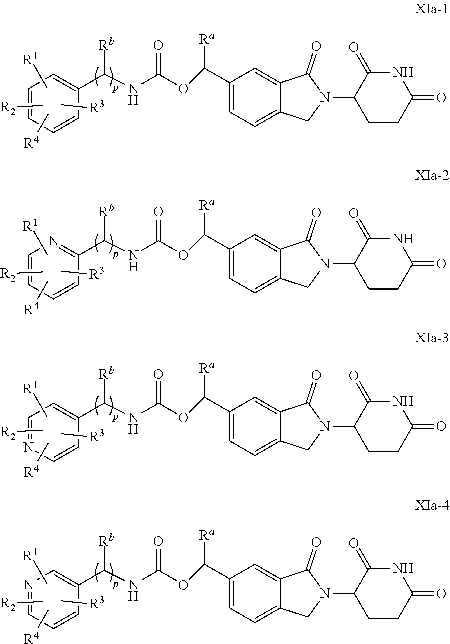

wherein
R¹, R², R³, R⁴ each are independently selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, or —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
p is 0 or 1, and R^{a}, R^{b} are independently of each other H or methyl.

In some embodiments of a compound of formula XIa-1, XIa-2, XIa-3, and XIa-4, p is 0. In some embodiments of a compound of formula XIa-1, XIa-2, XIa-3, and XIa-4, p is 1. In some embodiments of a compound of formula XIa-1, XIa-2, XIa-3, and XIa-4, p is 1 and R^{b} is H. In some embodiments of a compound of formula XIa-1, XIa-2, XIa-3, and XIa-4, p is 1 and R^{b} is methyl.

In some embodiments of a compound of formula XIa-1, XIa-2, XIa-3, and XIa-4, p is 0 and R^{a} is H. In some embodiments of a compound of formula XIa-1, XIa-2, XIa-3, and XIa-4, p is 0 and R^{a} is methyl. In some embodiments of a compound of formula XIa-1, XIa-2, XIa-3, and XIa-4, p is 1, R^{b} is H and R^{a} is H. In some embodiments of a compound of formula XIa-1, XIa-2, XIa-3, and XIa-4, p is 1, R^{b} is methyl and R^{a} is H.

In some embodiments of a compound of formula XIa-1, XIa-2, XIa-3, and XIa-4, R¹, R², R³, and R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, R¹, R², R³, and R⁴ are independently of each other selected from H, linear or branched —C₁₋₆ alkyl, —C₁₋₆ alkoxy, CF₃, CHF₂, CMeF₂, OCF₃, OCHF₂, —CN, and halogen, such as F, Cl or Br, e.g. F or Cl.

In some embodiments of a compound of formula XIa-1, XIa-2, XIa-3, and XIa-4, R¹ is H and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆ alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, R¹ is H and R², R³, R⁴ are independently of each other selected from H, linear or branched —C₁₋₆ alkyl, —C₁₋₆ alkoxy, CF₃, CHF₂, CMeF₂, OCF₃, OCHF₂, —CN, and halogen, such as F, Cl or Br, e.g. F or Cl.

In some embodiments of a compound of formula XIa-1, XIa-2, XIa-3, and XIa-4, n is 1, R^{a} is H or CH₃ and R¹, R², R³, and R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, n is 1, R^{a} is H or CH₃ and R¹, R², R³, and R⁴ are independently of each other selected from H, linear or branched —C₁₋₆ alkyl, —C₁₋₆ alkoxy, CF₃, CHF₂, CMeF₂, OCF₃, OCHF₂, —CN, and halogen, such as F, Cl or Br, e.g. F or Cl.

In some embodiments of a compound of formula XIa-1, XIa-2, XIa-3, and XIa-4, n is 1, R^{a} is H or CH₃, R¹ is H and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, n is 1, R^{a} is H or CH₃, R¹ is H and R², R³, R⁴ are independently of each other selected from H, linear or branched —C₁₋₆ alkyl, —C₁₋₆ alkoxy, CF₃, CHF₂, CMeF₂, OCF₃, OCHF₂, —CN, and halogen, such as F, Cl or Br, e.g. F or Cl.

In some embodiments of a compound of formula XIa-1, XIa-2, XIa-3, and XIa-4, p is 0 and R¹, R², R³, and R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆ alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, p is 0 and R¹, R², R³, and R⁴ are independently of each other selected from H, linear or branched —C₁₋₆ alkyl, —C₁₋₆ alkoxy, CF₃, CHF₂, CMeF₂, OCF₃, OCHF₂, —CN, and halogen, such as F, Cl or Br, e.g. F or Cl.

In some embodiments of a compound of formula XIa-1, XIa-2, XIa-3, and XIa-4, p is 0, R¹ is H and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, p is 0, R¹ is H and R², R³, R⁴ are independently of each other selected from H, linear or branched —C₁₋₆ alkyl, —C₁₋₆ alkoxy, CF₃, CHF₂, CMeF₂, OCF₃, OCHF₂, —CN, and halogen, such as F, Cl or Br, e.g. F or Cl.

In some embodiments of a compound of formula XIa-1, XIa-2, XIa-3, and XIa-4, p is 1, R^{b} is H or CH₃, R^{c} is H, and R¹, R², R³, and R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆ alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, p is 1, R^{b} is H or CH₃, R^{c} is H, and R¹, R², R³, and R⁴ are independently of each other selected from H, linear or branched —C₁₋₆ alkyl, —C₁₋₆ alkoxy, CF₃, CHF₂, CMeF₂, OCF₃, OCHF₂, —CN, and halogen, such as F, Cl or Br, e.g. F or Cl.

In some embodiments of a compound of formula XIa-1, XIa-2, XIa-3, and XIa-4, p is 1, R^{b} is H or CH₃, R^{c} is H, R¹ is H and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆ alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, p is 1, R^{b} is H or CH₃, R^{c} is H, R¹ is H and R², R³, R⁴ are independently of each other selected from H, linear or branched —C₁₋₆ alkyl, —C₁₋₆ alkoxy, CF₃, CHF₂, CMeF₂, OCF₃, OCHF₂, —CN, and halogen, such as F, Cl or Br, e.g. F or Cl.

In some embodiments of a compound of formula, XIa-1, XIa-2, XIa-3, and XIa-4, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe.

In some embodiments of a compound of formula XIa-1, XIa-2, XIa-3, and XIa-4, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀-aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is H; and p is 0.

In some embodiments of a compound of formula XIa-1, XIa-2, XIa-3, and XIa-4, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is H; and p is 0.

In some embodiments of a compound of formula XIa-1, XIa-2, XIa-3, and XIa-4, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is H; and p is 0.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is H; and p is 0.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is H; and p is 0.

In some embodiments of a compound of formula XIa-1, XIa-2, XIa-3, and XIa-4, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is CH₃; and p is 0.

In some embodiments of a compound of formula XIa-1, XIa-2, XIa-3, and XIa-4, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is CH₃; and p is 0.

In some embodiments of a compound of formula XIa-1, XIa-2, XIa-3, and XIa-4, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n is 1, R^{a} is CH₃; and p is 0.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is CH₃; and p is 0.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n is 1, R^{a} is CH₃; and p is 0.

In some embodiments of a compound of formula XIa-1, XIa-2, XIa-3, and XIa-4, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{b}, R^{c} are H.

In some embodiments of a compound of formula XIa-1, XIa-2, XIa-3, and XIa-4, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{b}, R^{c} are H.

In some embodiments of a compound of formula XIa-1, XIa-2, XIa-3, and XIa-4, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{b}, R^{c} are H.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n, p are 1, R^{a}, R^{b}, R^{c} are H.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n, p are 1, R^{a}, R^{b}, R^{c} are H.

In some embodiments of a compound of formula XIa-1, XIa-2, XIa-3, and XIa-4, R¹ is C₃₋₆ cycloalkyl, —C₁₋₄ alkyl-C₃₋₆ cycloalkyl, —O—C₃₋₆ cycloalkyl, —C₁₋₄ alkoxy-C₃₋₆ cycloalkyl, C₆₋₁₀ aryl, —C₁₋₄ alkyl-C₆₋₁₀ aryl, —O—C₆₋₁₀ aryl, —C₁₋₄ alkoxy-C₆₋₁₀ aryl, 5-10 membered heteroaryl, —C₁₋₄ alkyl-(5-10 membered heteroaryl), —O-(5-10 membered heteroaryl), —C₁₋₄ alkoxy-(5-10 membered heteroaryl), 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{c} are H, R^{b} is CH₃.

In some embodiments of a compound of formula XIa-1, XIa-2, XIa-3, and XIa-4, R¹ is C₃₋₆ cycloalkyl, —O—C₃₋₆ cycloalkyl, C₆₋₁₀ aryl, 5-10 membered heteroaryl, 4-8 membered heterocycloalkyl, —C₁₋₄ alkyl-(4-8 membered heterocycloalkyl), —O-(4-8 membered heterocycloalkyl), —C₁₋₄ alkoxy-(4-8 membered heterocycloalkyl), wherein R¹ is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{c} are H, R^{b} is CH₃.

In some embodiments of a compound of formula XIa-1, XIa-2, XIa-3, and XIa-4, R¹ is is a group of formula -L³-X², wherein L³ is a covalent bond, linear or branched C₁₋₄ alkyl, —O—, —C₁₋₄ alkoxy and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy; and R², R³, R⁴ are independently of each other selected from H, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —C₁₋₆ alkylC(O)OH, —C₁₋₆alkylC(O)O—C₁₋₆alkyl, NH₂, —C₁₋₆ alkylhydroxy, and halogen, such as F, Cl or Br, e.g. F or Cl; and n, p are 1, R^{a}, R^{c} are H, R^{b} is CH₃.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, cyclobutyl, C₆ aryl, pyridinyl, pyrrolidinyl, piperdinyl, morpholinyl, oxetanyl, piperazinyl, azetidinyl, 2-oxa-5-azabicyclo[2.2.1]heptanyl, 8-oxa-3-azabicyclo[3.2.1]octan-3-yl, wherein X² is unsubstituted or substituted with one or more of linear or branched C₁₋₄ alkyl, —C₁₋₄ alkoxy, e.g. —OMe, NMe₂, halogen, e.g. F; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n, p are 1, R^{a}, R^{c} are H, R^{b} is CH₃.

In some embodiments of a compound of formula XIa-1, XIa-2, XIa-3, and XIa-4, R¹ is is a group of formula -L³-X², wherein L³ is a covalent bond, —CH₂—, —O—, —OCH₂—, —O(CH₂)₂— and X² is cyclopropyl, methyl-cyclopropyl, fluoro-cyclopropyl, difluoro-cyclopropyl, cyclobutyl, C₆ aryl, methyl-C₆ aryl, fluoro-C₆ aryl, methoxy-C₆ aryl, pyridinyl, pyrrolidinyl, N-methyl-pyrrolidinyl, methyl-pyrrolidinyl, piperdinyl, N-methyl piperdinyl, methyl-piperdinyl, difluoro-piperidinyl, morpholinyl, N-methyl-morpholinyl, oxetanyl, methyl-oxetanyl, piperazinyl, N-methyl-piperazinyl, azetidinyl, methyl-azetidinyl, N-dimethyl-azetidinyl, 2-oxa-5-azabicyclo[2.2.1]heptanyl, 8-oxa-3-azabicyclo[3.2.1]octan-3-yl; and R², R³, R⁴ are independently of each other selected from H, halogen, e.g. Cl, F, linear or branched —C₁₋₄ alkyl, e.g., Me, Et, t-But, CF₃, CHF₂, CMeF₂, —OCF₃, OCHF₂, CN, and C₁₋₄ alkoxy, e.g. —OMe; and n, p are 1, R^{a}, R^{c} are H, R^{b} is CH₃.

In some embodiments of a compound or pharmaceutically acceptable salts or stereoisomers thereof of formula XIa-1, XIa-2, XIa-3, and XIa-4, R¹, R², R³ are defined as above and R⁴ is H such that the aromatic ring contains 4 substituents which are not H.

In some embodiments of a compound or pharmaceutically acceptable salts or stereoisomers thereof of formula XIa-1, XIa-2, XIa-3, and XIa-4, R¹ and R² are defined as above and R³ and R⁴ each are H, such that the aromatic ring contains 3 substituents which are not H.

More specific embodiments of the compound of formula XI are also provided by formula XIa-5, XIa-6, XIa-7, XIa-8, XIa-9, XIa-10, wherein two of w¹ to w⁵ are N, e.g., wherein w¹, w² are N, w³ to w⁵ are C; or w¹, w⁵ are N, w² to w⁴ are C; or w², w⁴ are N, w¹, w³, w⁵ are C; or w¹, w³ are N, w², w⁴, w⁵ are C; or w², w³ are N, w¹, w⁴, w⁵ are C; or w¹, w⁴ are N, w², w³, w⁵ are C

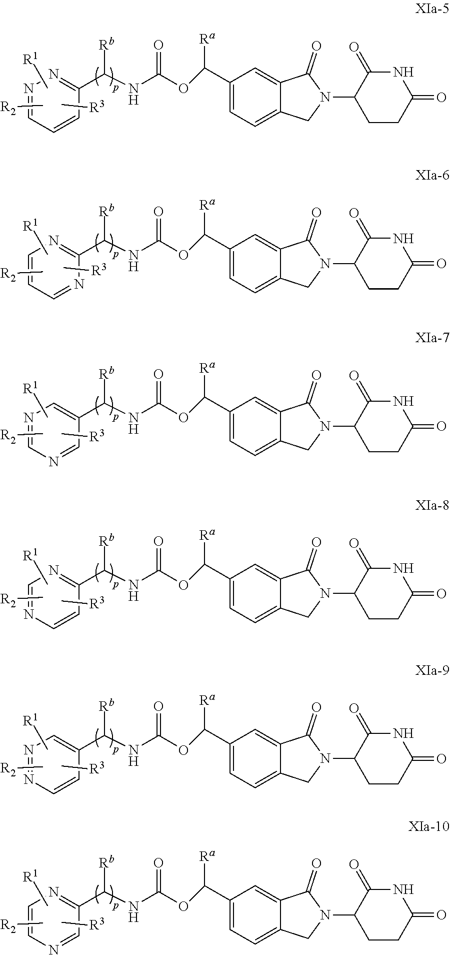

wherein
R¹, R², R³ each are independently selected from H, linear or branched C₁₋₄ alkyl, C₁₋₄ alkoxy, 4-8 membered heterocycloalkyl, —C₁₋₄ alkyl 4-8 membered heterocycloalkyl, —C₁₋₄ alkoxy 4-8 membered heterocycloalkyl, —O-(4-8 membered heterocycloalkyl), CF₃, CHF₂, CMeF₂, —O—(CH₂)₂—OMe, OCF₃, OCHF₂, C₁₋₆ alkylamino, —CN, —OC(O)—C₁₋₆alkyl, —N(H)C(O)—C₁₋₆ alkyl, —C(O)O—C₁₋₆alkyl, —COOH, —C₁₋₆alkylC(O)OH, —C₁₋₆alkylC(O)O—C₁₋₆alkyl, NH₂, C₁₋₆ alkoxy, —C₁₋₆ alkylhydroxy, and halogen, such as F, Cl or Br, e.g. F or Cl, and C₆ aryl, such as phenyl, wherein the 4-8 membered heterocycloalkyl may be unsubstituted or substituted with C₁₋₄ alkyl, such as methyl, ethyl, C₁₋₄ alkoxy, such as methoxy, ethoxy, N(Me)₂ and halogen, such as F, Cl or Br, e.g. F, Cl; R^{a}, R^{b} are independently of each other H or methyl and p is 0 or 1.

In some embodiments of a compound of formula XIa-5, XIa-6, XIa-7, XIa-8, XIa-9, XIa-10, p is 0. In some embodiments of a compound of formula XIa-5, XIa-6, XIa-7, XIa-8, XIa-9, XIa-10, p is 1.

In some embodiments of a compound of formula XIa-5, XIa-6, XIa-7, XIa-8, XIa-9, XIa-10, R^{a} is H. In some embodiments of a compound of formula XIa-5, XIa-6, XIa-7, XIa-8, XIa-9, XIa-10, R^{a} is methyl. In some embodiments of a compound of formula XIa-5, XIa-6, XIa-7, XIa-8, XIa-9, XIa-10, p is 0 and R^{a} is H. In some embodiments of a compound of formula XIa-5, XIa-6, XIa-7, XIa-8, XIa-9, XIa-10, p is 0 and R^{a} is methyl.

In some embodiments of a compound of formula XIa-5, XIa-6, XIa-7, XIa-8, XIa-9, XIa-10, p is 1 and R^{b} is H. In some embodiments of a compound of formula XIa-5, XIa-6, XIa-7, XIa-8, XIa-9, XIa-10, p is 1 and R^{b} is methyl. In some embodiments of a compound of formula XIa-5, XIa-6, XIa-7, XIa-8, XIa-9, XIa-10, p is 1, R^{b} is H and R^{a} is H. In some embodiments of a compound of formula XIa-5, XIa-6, XIa-7, XIa-8, XIa-9, XIa-10, p is 1, R^{b} is methyl and R^{a} is H.

In some embodiments of a compound of formula XIa-5, XIa-6, XIa-7, XIa-8, XIa-9, XIa-10, R¹ is selected from azetidinyl, NMe₂-azetidinyl, pyrrolidinyl, Me-pyrrolidinyl, piperidinyl, Me-piperidinyl, Di-F-piperidinyl, N-Me-piperazinyl, morpholinyl, 2-oxa-5-azabicyclo[2.2.1]heptanyl, 8-oxa-3-azabicyclo[3.2.1]octan-3-yl: and R², R³ each are independently selected from H, linear or branched C₁₋₄ alkyl, C₁₋₄ alkoxy, CF₃, CHF₂, CMeF₂, —O—(CH₂)₂—OMe, OCF₃, OCHF₂, C₁₋₆ alkylamino, —CN, —CNMe₂, —C₁₋₆ alkylhydroxy, and halogen, such as F, Cl or Br, e.g. F or Cl.

In some embodiments, the present disclosure is directed towards a compound containing a fused 6(saturated)-6(aromatic) ring system or a fused 5(saturated)-6(aromatic) ring system of formula XIb:

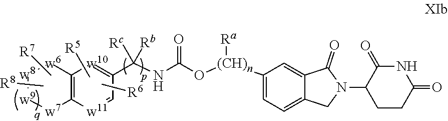

wherein
one or two of w⁶, w⁷, w⁸, w⁹ are selected from C and O and the remaining of w⁶, w⁷, w⁸, w⁹ are C; w¹⁰, w¹¹ are independently of each other selected from C and N;
R⁵, R⁶, R⁷ R⁸ are independently of each other selected from H, linear or branched C₁₋₄ alkyl, halogen, such as F or Cl, e.g. F; R^{a} is H, linear or branched C₁₋₄ alkyl, R^{b}, R^{c} are independently of each other H, linear or branched C₁₋₄ alkyl, q is 0, 1; n is 1, or 2; and p is 0, 1, or 2.

In some embodiments of a compound of formula XIb, R^{a} is H. In some embodiments of a compound of formula XIb, R^{a} is methyl. In some embodiments of a compound of formula XIb, n is 1. In some embodiments of a compound of formula XIb, n is 1 and R^{a} is H. In some embodiments of a compound of formula XIb, n is 1 and R^{a} is methyl.

In some embodiments of a compound of formula XIb, p is 0. In some embodiments of a compound of formula XIb, p is 0 and R^{a} is H. In some embodiments of a compound of formula XIb, p is 0 and R^{a} is methyl. In some embodiments of a compound of formula XIb, p is 1. In some embodiments of a compound of formula XIb, p is 1, and R^{b} and R^{c} are H. In some embodiments of a compound of formula XIb, p is 1, R^{b} is methyl and R^{c} is H.

In some embodiments of a compound of formula XIb, one of w¹⁰ and w¹¹ is C. In some embodiments of a compound of formula XIb, one of w¹⁰ and w¹¹ is C and the other is N.

In some embodiments of a compound of formula XIb, q is 0 and w⁸ is C. In some embodiments of a compound of formula XIb, q is 0, w⁸ is C and w⁶, w⁷ are selected from C and O. In some embodiments of a compound of formula XIb, q is 0, w⁸ is C and w⁶, w⁷ are O. In some embodiments of a compound of formula XIb, q is 0, w⁸ is C and one of w⁶, w⁷ is C and the other of w⁶, w⁷ is O.

In some embodiments of a compound of formula XIb, q is 1, and w⁶, w⁷, w⁸, w⁹ are C. In some embodiments of a compound of formula XIb, q is 1, and w⁶ is O and w⁷, w⁸, w⁹ are C. In some embodiments of a compound of formula XIb, q is 1, and w⁷ is O and w⁶, w⁸, w⁹ are C. In some embodiments of a compound of formula XIb, q is 1, and w is O and w⁶, w⁷, W⁹ are C. In some embodiments of a compound of formula XIb, q is 1, and w⁹ is O and w⁶, w⁷, w⁸ are C.

In some embodiments of a compound of formula XIb, R⁵, R⁶ are H.

In some embodiments of a compound of formula XIb, R⁷ R⁸ are independently of each other selected from H, linear or branched C₁₋₄ alkyl, halogen, such as F or Cl, e.g. F. R⁷ R⁸ may be attached to the same ring atom or to different ring atoms.

Some embodiments of a compound of formula XIb are provided by formula XIb′

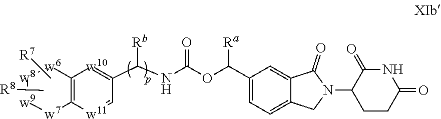

wherein
one or two of w⁶, w⁷, w⁸, w⁹ are selected from C and O and the remaining of w⁶, w⁷, w⁸, w⁹ are C; w¹⁰, w¹¹ are independently of each other selected from C and N;
R⁷, R⁸ are independently of each other selected from H, linear or branched C₁₋₄ alkyl, halogen, such as F or Cl, e.g. F; R^{a}, R^{b} are independently of each other H, linear or branched C₁₋₄ alkyl, and p is 0 or 1.

In some embodiments of a compound of formula XIb′, p is 0. In some embodiments of a compound of formula XIb′, p is 1.

In some embodiments of a compound of formula XIb′, R^{a} is H. In some embodiments of a compound of formula XIb′, R^{a} is methyl. In some embodiments of a compound of formula XIb′, p is 0 and R^{a} is H. In some embodiments of a compound of formula XIb′, p is 0 and R^{a} is methyl. In some embodiments of a compound of formula XIb′, p is 1 and R^{b} is H. In some embodiments of a compound of formula XIb′, p is 1 and R^{b} is methyl.

In some embodiments of a compound of formula XIb′, one of w¹⁰ and w¹¹ are C. In some embodiments of a compound of formula XIb′, one of w¹⁰ and w¹¹ is C and the other is N.

In some embodiments of a compound of formula XIb′, w⁶, w⁷, w⁸, w⁹ are C. In some embodiments of a compound of formula XIb′, w⁶ is O and w⁷, w⁸, w⁹ are C. In some embodiments of a compound of formula XIb′, w⁷ is O and w⁶, w⁸, w⁹ are C. In some embodiments of a compound of formula XIb′, w⁸ is O and w⁶, w⁷, w⁹ are C. In some embodiments of a compound of formula XIb′, w⁹ is O and w⁶, w⁷, w⁸ are C.

In some embodiments of a compound of formula XIb′, R⁷ R⁸ are independently of each other selected from H, linear or branched C₁₋₄ alkyl, halogen, such as F or Cl, e.g. F. R⁷ R⁸ may be attached to the same ring atom or to different ring atoms.

Some embodiments of a compound of formula XIb and XIb′ are provided by formula XIb-1, XIb-2

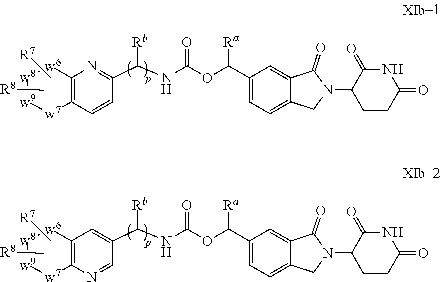

wherein
w⁶, w⁷, w⁸, w⁹ are independently of each other selected from C and O;
R⁷ R⁸ are independently of each other selected from H, linear or branched C₁₋₄ alkyl, halogen, such as F or Cl, e.g. F;
R^{a}, R^{b} are independently of each other H, linear or branched C₁₋₄ alkyl, and p is 0 or 1.

In some embodiments of a compound of formula XIb-1, XIb-2, p is 0. In some embodiments of a compound of formula XIb-1, XIb-2, p is 1.

In some embodiments of a compound of formula XIb-1, XIb-2, R^{a} is H. In some embodiments of a compound of formula XIb-1, XIb-2, R^{a} is methyl. In some embodiments of a compound of formula XIb-1, XIb-2, p is 0 and R^{a} is H. In some embodiments of a compound of formula XIb-1, XIb-2, p is 0 and R^{a} is methyl.

In some embodiments of a compound of formula XIb-1, XIb-2, p is 1 and R^{b} is H. In some embodiments of a compound of formula XIb-1, XIb-2, p is 1 and R^{b} is methyl. In some embodiments of a compound of formula XIb-1, XIb-2, p is 1, R^{b} is H and R^{a} is H. In some embodiments of a compound of formula XIb-1, XIb-2, p is 1, R^{b} is methyl and R^{a} is H.

In some embodiments of a compound of formula XIb-1 or XIb-2, w⁶, w⁷, w⁸, w⁹ are C. In some embodiments of a compound of formula XIb-1 or XIb-2, w⁶ is O and w⁷, w⁸, w⁹ are C. In some embodiments of a compound of formula XIb-1 or XIb-2, w⁷ is O and w⁶ w⁸, w⁹ are C. In some embodiments of a compound of formula XIb-1 or XIb-2, w⁸ is O and w⁶, w⁷, w⁹ are C. In some embodiments of a compound of formula XIb-1 or XIb-2, w⁹ is O and w⁶, w⁷, w⁸ are C.

In some embodiments of a compound of formula XIb-1 or XIb-2, R⁷ R⁸ are independently of each other selected from H, linear or branched C₁₋₄ alkyl, halogen, such as F or Cl, e.g. F. R⁷ R⁸ may be attached to the same ring atom or to different ring atoms.

Some embodiments of a compound of formula XIb-1 are provided by formula XIb-1a, XIb-1b, XIb-1c, and XIb-1d

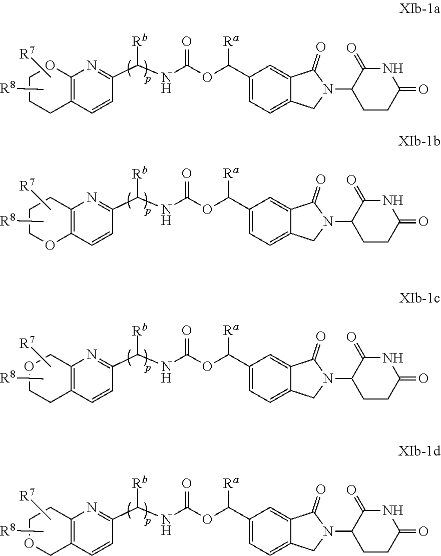

wherein
R⁷ R⁸ are independently of each other selected from H, linear or branched C₁₋₄ alkyl, halogen, such as F or Cl, e.g. F;
R^{a}, R^{b} are independently of each other H, linear or branched C₁₋₄ alkyl, and p is 0 or 1.

In some embodiments of a compound of formula XIb-1a, XIb-1b, XIb-1c, and XIb-1d, p is 0. In some embodiments of a compound of formula XIb-1a, XIb-1b, XIb-1c, and XIb-1d, p is 1.

In some embodiments of a compound of formula XIb-1a, XIb-1b, XIb-1c, and XIb-1d, R^{a} is H. In some embodiments of a compound of formula XIb-1a, XIb-1b, XIb-1c, and XIb-1d, R^{a} is methyl. In some embodiments of a compound of formula XIb-1a, XIb-1b, XIb-1c, and XIb-1d, p is 0 and R^{a} is H. In some embodiments of a compound of formula XIb-1a, XIb-1b, XIb-1c, and XIb-1d, p is 0 and R^{a} is methyl.

In some embodiments of a compound of formula XIb-1a, XIb-1b, XIb-1c, and XIb-1d, p is 1 and R^{b} is H. In some embodiments of a compound of formula XIb-1a, XIb-1b, XIb-1c, and XIb-1d, p is 1 and R^{b} is methyl. In some embodiments of a compound of formula XIb-1a, XIb-1b, XIb-1c, and XIb-1 d, p is 1, R^{b} is H and R^{a} is H. In some embodiments of a compound of formula XIb-1a, XIb-1b, XIb-1c, and XIb-1d, p is 1, R^{b} is methyl and R^{a} is H.

In some embodiments of a compound of formula XIb-1a, XIb-1b, XIb-1c, and XIb-1d, R⁷ R⁸ are independently of each other selected from H, linear or branched C₁₋₄ alkyl, halogen, such as F or Cl, e.g. F. R⁷ R⁸ may be attached to the same ring atom or to different ring atoms.

Some embodiments of a compound of formula XIb-2 are provided by formula XIb-2a, XIb-2b, XIb-2c, or XIb-2d

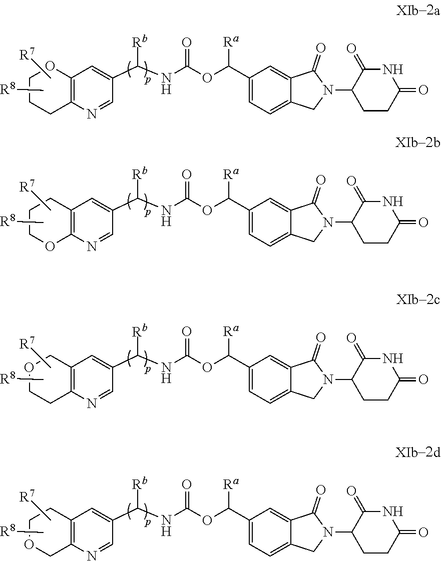

wherein
R⁷ R⁸ are independently of each other selected from H, linear or branched C₁₋₄ alkyl, halogen, such as F or Cl, e.g. F;
R^{a}, R^{b} are independently of each other H, linear or branched C₁₋₄ alkyl, and p is 0 or 1.

In some embodiments of a compound of formula XIb-2a, XIb-2b, XIb-2c, or XIb-2d, p is 0. In some embodiments of a compound of formula XIb-2a, XIb-2b, XIb-2c, or XIb-2d, p is 1.

In some embodiments of a compound of formula XIb-2a, XIb-2b, XIb-2c, or XIb-2d, R^{a} is H. In some embodiments of a compound of formula XIb-2a, XIb-2b, XIb-2c, or XIb-2d, R^{a} is methyl. In some embodiments of a compound of formula XIb-2a, XIb-2b, XIb-2c, or XIb-2d, p is 0 and R^{a} is H. In some embodiments of a compound of formula XIb-2a, XIb-2b, XIb-2c, or XIb-2d, p is 0 and R^{a} is methyl.

In some embodiments of a compound of formula XIb-2a, XIb-2b, XIb-2c, or XIb-2d, p is 1 and R^{b} is H. In some embodiments of a compound of formula XIb-2a, XIb-2b, XIb-2c, or XIb-2d, p is 1 and R^{b} is methyl. In some embodiments of a compound of formula XIb-2a, XIb-2b, XIb-2c, or XIb-2d, p is 1, R^{b} is H and R^{a} is H. In some embodiments of a compound of formula XIb-2a, XIb-2b, XIb-2c, or XIb-2d, p is 1, R^{b} is methyl and R^{a} is H.

In some embodiments of a compound of formula XIb-2a, XIb-2b, XIb-2c, or XIb-2d, R⁷ R⁸ are independently of each other selected from H, linear or branched C₁₋₄ alkyl, halogen, such as F or Cl, e.g. F. R⁷ R⁸ may be attached to the same ring atom or to different ring atoms.

In some embodiments the compound of formula XIa, q is 0 and is provided by a compound of formula XIb-3

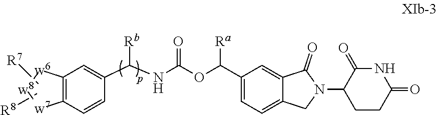

wherein
w⁶, w⁷, w⁸ are independently of each other selected from C and O;
R⁷ R⁸ are independently of each other selected from H, linear or branched C₁₋₄ alkyl, halogen, such as F or Cl, e.g. F;
R^{a}, R^{b} are independently of each other H, linear or branched C₁₋₄ alkyl and p is 0 or 1.

In some embodiments of a compound of formula XIb-3, p is 0. In some embodiments of a compound of formula XIb-3, p is 1.

In some embodiments of a compound of formula XIb-3, R^{a} is H. In some embodiments of a compound of formula XIb-3, R^{a} is methyl. In some embodiments of a compound of formula XIb-3, p is 0 and R^{a} is H. In some embodiments of a compound of formula XIb-3, p is 0 and R^{a} is methyl.

In some embodiments of a compound of formula XIb-3, p is 1 and R^{b} is H. In some embodiments of a compound of formula XIb-3, p is 1 and R^{b} is methyl. In some embodiments of a compound of formula XIb-3, p is 1, R^{b} is H and R^{a} is H. In some embodiments of a compound of formula XIb-3, p is 1, R^{b} is methyl and R^{a} is H.

In some embodiments of a compound of formula XIb-3, w⁸ is C. In some embodiments of a compound of formula XIb-3, w⁸ is C and w⁶, w⁷ are selected from C and O. In some embodiments of a compound of formula XIb-3, w⁸ is C and w⁶, w⁷ are O. In some embodiments of a compound of formula XIb-3, w⁸ is C and one of w⁶, w⁷ is C and the other of w⁶, w⁷ is O.

In some embodiments of a compound of formula XIb-3, R⁷ R⁸ are independently of each other selected from H, linear or branched C₁₋₄ alkyl, halogen, such as F or Cl, e.g. F. R⁷ R⁸ may be attached to the same ring atom or to different ring atoms.

In some embodiments the compound of formula XIb and XIb-3 is provided by formula XIb-3a, XIb-3b, XIb-3c, and XIb-3d

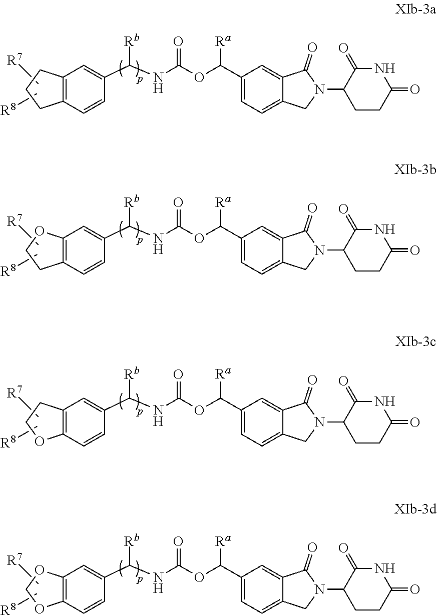

wherein
R⁷ R⁸ are independently of each other selected from H, linear or branched C₁₋₄ alkyl, halogen, such as F or Cl, e.g. F;
R^{a}, R^{b} are independently of each other H, linear or branched C₁₋₄ alkyl, and p is 0 or 1.

In some embodiments of a compound of formula XIb-3a, XIb-3b, XIb-3c, and XIb-3d, p is 0. In some embodiments of a compound of formula XIb-3a, XIb-3b, XIb-3c, and XIb-3d, p is 1.

In some embodiments of a compound of formula XIb-3a, XIb-3b, XIb-3c, and XIb-3d, R^{a} is H. In some embodiments of a compound of formula XIb-3a, XIb-3b, XIb-3c, and XIb-3d, R^{a} is methyl. In some embodiments of a compound of formula XIb-3a, XIb-3b, XIb-3c, and XIb-3d, p is 0 and R^{a} is H. In some embodiments of a compound of formula XIb-3a, XIb-3b, XIb-3c, and XIb-3d, p is 0 and R^{a} is methyl.

In some embodiments of a compound of formula XIb-3a, XIb-3b, XIb-3c, and XIb-3d, p is 1 and R^{b} is H. In some embodiments of a compound of formula XIb-3a, XIb-3b, XIb-3c, and XIb-3d, p is 1 and R^{b} is methyl. In some embodiments of a compound of formula XIb-3a, XIb-3b, XIb-3c, and XIb-3d, p is 1, R^{b} is H and R^{a} is H. In some embodiments of a compound of formula XIb-3a, XIb-3b, XIb-3c, and XIb-3d, p is 1, R^{b} is methyl and R^{a} is H.

In some embodiments of a compound of formula XIb-3a, XIb-3b, XIb-3c, and XIb-3d, R⁷ R⁸ are independently of each other selected from H, linear or branched C₁₋₄ alkyl, halogen, such as F or Cl, e.g. F. R⁷ R⁸ may be attached to the same ring atom or to different ring atoms.

In some embodiments, the present disclosure is directed towards a compound of formula XIc:

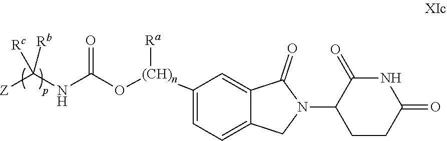

wherein
Z is H, linear or branched —C₁₋₆ alkyl, —C₃₋₆ cycloalkyl, 4-8 membered heterocycloalkyl, wherein Z is unsubstituted or substituted with linear or branched C₁₋₄ alkyl, C₆ aryl, C₆ aryloxy, 6 membered heteroaryl or CF₃;
or Z together with the N atom of the carbamate forms a 4-8 membered heterocycloalkyl, which is unsubstituted or substituted with linear or branched C₁₋₄ alkyl, C₆ aryl, C₆ aryloxy, 6 membered heteroaryl or CF₃;
R^{a} is H, linear or branched C₁₋₄ alkyl, such as methyl; R^{b}, R^{c} are independently of each other H, linear or branched C₁₋₄ alkyl, such as methyl;
n is 1, or 2; and p is 0, 1, or 2.

In some embodiments of a compound of formula XIc, R^{a} is H. In some embodiments of a compound of formula XIc, R^{a} is methyl. In some embodiments of a compound of formula XIc, n is 1. In some embodiments of a compound of formula XIc, n is 1 and R^{a} is H. In some embodiments of a compound of formula XIc, n is 1 and R^{a} is methyl.

In some embodiments of a compound of formula XIc, p is 0. In some embodiments of a compound of formula XIc, p is 1. In some embodiments of a compound of formula XIc, p is 1, and R^{b} and R^{c} are H. In some embodiments of a compound of formula XIc, p is 1, R^{b} is methyl and R^{c} is H.

In some embodiments of a compound of formula XIc, n is 1 and p is 1. In some embodiments of a compound of formula XIc, n is 1, p is 1, R^{a} is H and R^{b}, R^{c} are H. In some embodiments of a compound of formula XIc, n is 1, p is 1, R^{a} is methyl and R^{b}, R^{c} are H.

In some embodiments of a compound of formula XIc, n is 1 and p is 0. In some embodiments of a compound of formula XIc, n is 1, p is 0 and R^{a} is H. In some embodiments of a compound of formula XIc, n is 1, p is 0 and R^{a} is methyl.

In some embodiments of a compound of formula XIc, Z is linear or branched —C₁₋₆ alkyl, —C₃₋₆ cycloalkyl, 4-6 membered heterocycloalkyl, wherein Z is unsubstituted or substituted with linear or branched C₁₋₄ alkyl, C₆ aryl, C₆ aryloxy, 6 membered heteroaryl or CF₃; or Z together with the N atom of the carbamate forms a 4-6 membered heterocycloalkyl, which is unsubstituted or substituted with C₁₋₄ alkyl, C₆ aryl, C₆ aryloxy, 6 membered heteroaryl or CF₃.

In some embodiments of a compound of formula XIc, Z is linear or branched —C₁₋₆ alkyl, —C₃₋₆ cycloalkyl, 4-6 membered heterocycloalkyl, wherein Z is unsubstituted or substituted with linear or branched C₁₋₄ alkyl, C₆ aryl, C₆ aryloxy, 6 membered heteroaryl or CF₃; or Z together with the N atom of the carbamate forms a pyrrolidinyl, piperdinyl, morpholinyl, piperazinyl, N-methyl piperazinyl, which is unsubstituted or substituted with C₁₋₄ alkyl, C₆ aryl, C₆ aryloxy, 6 membered heteroaryl or CF₃.

In some embodiments of a compound of formula XIc, Z is linear or branched C₁₋₆ alkyl, C₃₋₆ cycloalkyl, pyrrolidinyl, piperdinyl, wherein Z is unsubstituted or substituted with C₁₋₄ alkyl, phenyl, phenoxy, pyridinyl or CF₃; or Z together with the N atom of the carbamate forms a pyrrolidinyl, piperdinyl, morpholinyl, piperazinyl, N-methyl piperazinyl, which is unsubstituted or substituted with C₁₋₄ alkyl, phenyl, phenoxy, pyridinyl or CF₃.

In some embodiments of a compound of formula XIc, n is 1 and p is 0. Thus, in some embodiments a compound of formula XIc is a compound or pharmaceutically acceptable salts or stereoisomers thereof of formula XIc-1 and XIc-1a:

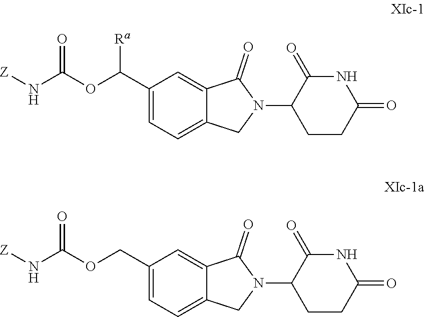

wherein
Z is H, —C₃₋₆ cycloalkyl, 4-8 membered heterocycloalkyl, wherein Z is unsubstituted or substituted with linear or branched C₁₋₄ alkyl, C₆ aryl, C₆ aryloxy, 6 membered heteroaryl or CF₃; and R^{a} is H, linear or branched C₁₋₄ alkyl, such as methyl.

In some embodiments of a compound of formula XIc-1 and XIc-1a, Z is C₃₋₆ cycloalkyl, 4-6 membered heterocycloalkyl, wherein Z is unsubstituted or substituted with C₁₋₄ alkyl, C₆ aryl, C₆ aryloxy, 6 membered heteroaryl or CF₃.

In some embodiments of a compound of formula XIc-1 and XIc-1a, Z is C₃₋₆ cycloalkyl, 5-6 membered heterocycloalkyl, wherein Z is unsubstituted or substituted with C₁₋₄ alkyl, C₆ aryl, C₆ aryloxy, 6 membered heteroaryl or CF₃.

In some embodiments of a compound of formula XIc-1 and XIc-1a, Z is cyclopropyl, cyclobutyl, cyclopentyl, cycohexyl, pyrrolidinyl, wherein Z is unsubstituted or substituted with linear or branched C₁₋₄ alkyl, such as methyl, ethyl, t-butyl, phenyl, pyridinyl, pyrazinyl or CF₃.

In some embodiments of a compound of formula XIc, n is 1 and p is 1. Thus, in some embodiments a compound of formula XIc is a compound or a pharmaceutically acceptable salt or stereoisomer thereof of formula XIc-2 and XIc-2a:

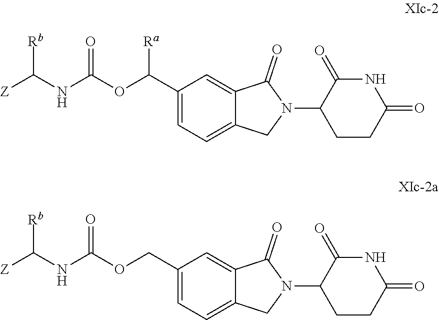

wherein
Z is H, linear or branched —C₁₋₆ alkyl, —C₃₋₆ cycloalkyl, 4-8 membered heterocycloalkyl, wherein Z is unsubstituted or substituted with C₁₋₄ alkyl, C₆ aryl, C₆ aryloxy, 6 membered heteroaryl or CF₃; or Z together with the N atom of the carbamate forms a 4-8 membered heterocycloalkyl, which is unsubstituted or substituted with C₁₋₄ alkyl, C₆ aryl, C₆ aryloxy, 6 membered heteroaryl or CF₃;
R^{a} is H, linear or branched C₁₋₄ alkyl, such as methyl;
R^{b} is H, linear or branched C₁₋₄ alkyl, such as methyl.

In some embodiments of a compound of formula XIc-1, R^{a} is H. In some embodiments of a compound of formula XIc-1, R^{a} is methyl.

In some embodiments of a compound of formula XIc-1, R^{b} is H. In some embodiments of a compound of formula XIc-1, R^{b} is methyl.

In some embodiments of a compound of formula XIc-1, Z is linear or branched —C₁₋₆ alkyl, —C₃₋₆ cycloalkyl, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the carbamate forms a 4-6 membered heterocycloalkyl, which is unsubstituted or substituted with C₁₋₄ alkyl, C₆ aryl, C₆ aryloxy, 6 membered heteroaryl or CF₃.

In some embodiments of a compound of formula XIc-1, Z is linear or branched —C₁₋₆ alkyl, —C₃₋₆ cycloalkyl, 4-6 membered heterocycloalkyl, wherein Z is unsubstituted or substituted with C₁₋₄ alkyl, C₆ aryl, C₆ aryloxy, 6 membered heteroaryl or CF₃; or Z together with the N atom of the carbamate forms a pyrrolidinyl, piperdinyl, morpholinyl, piperazinyl, N-methyl piperazinyl, which is unsubstituted or substituted with C₁₋₄ alkyl, C₆ aryl, C₆ aryloxy, 6 membered heteroaryl or CF₃.

In some embodiments of a compound of formula XIc-1, Z is linear or branched C₁₋₆ alkyl, C₃₋₆ cycloalkyl, pyrrolidinyl, piperdinyl, wherein Z is unsubstituted or substituted with C₁₋₄ alkyl, phenyl, phenoxy, pyridinyl or CF₃; or Z together with the N atom of the carbamate forms a pyrrolidinyl, piperdinyl, morpholinyl, piperazinyl, N-methyl piperazinyl, which is unsubstituted or substituted with C₁₋₄ alkyl, phenyl, phenoxy, pyridinyl or CF₃.

In some embodiments, the present disclosure is directed towards a compound or a pharmaceutically acceptable salt or stereoisomer thereof of formula XIIa or XIIb

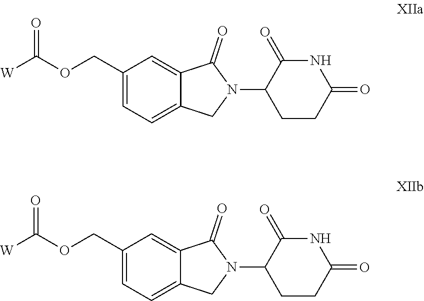

wherein W is selected from

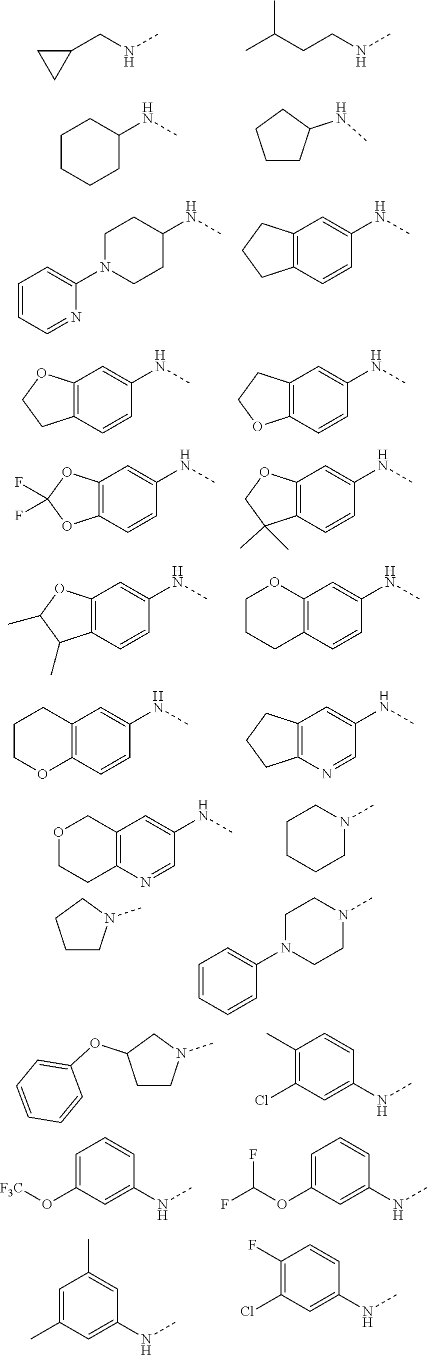

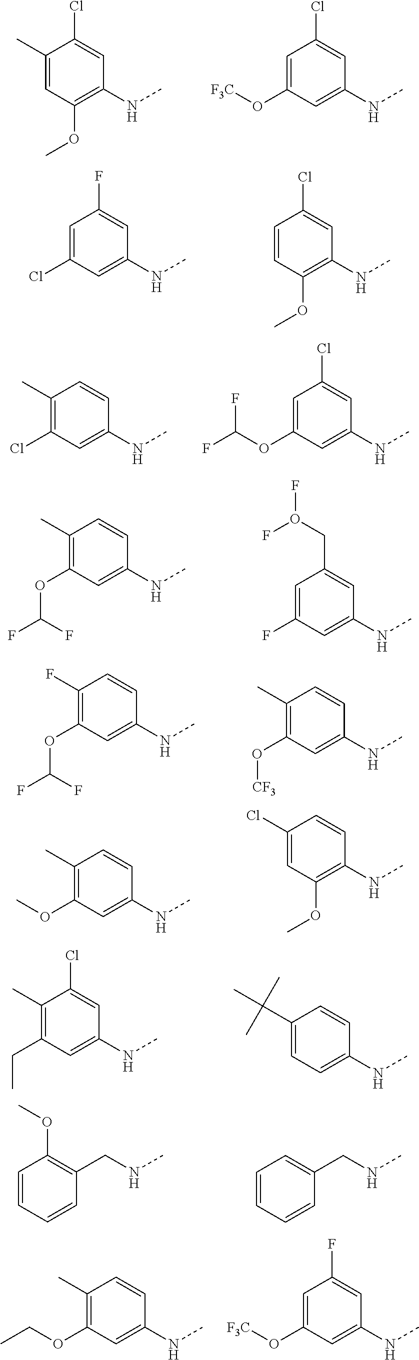

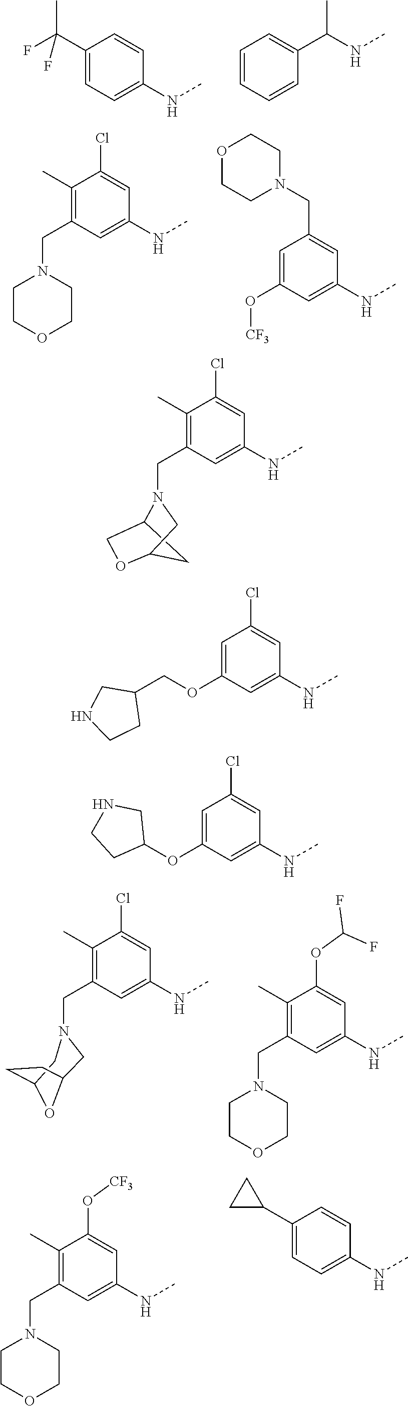

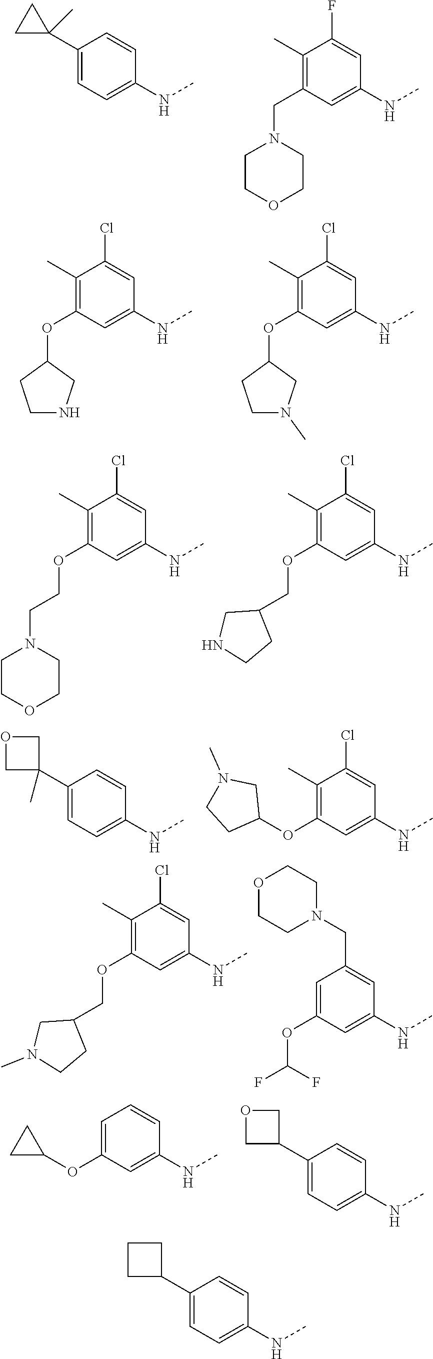

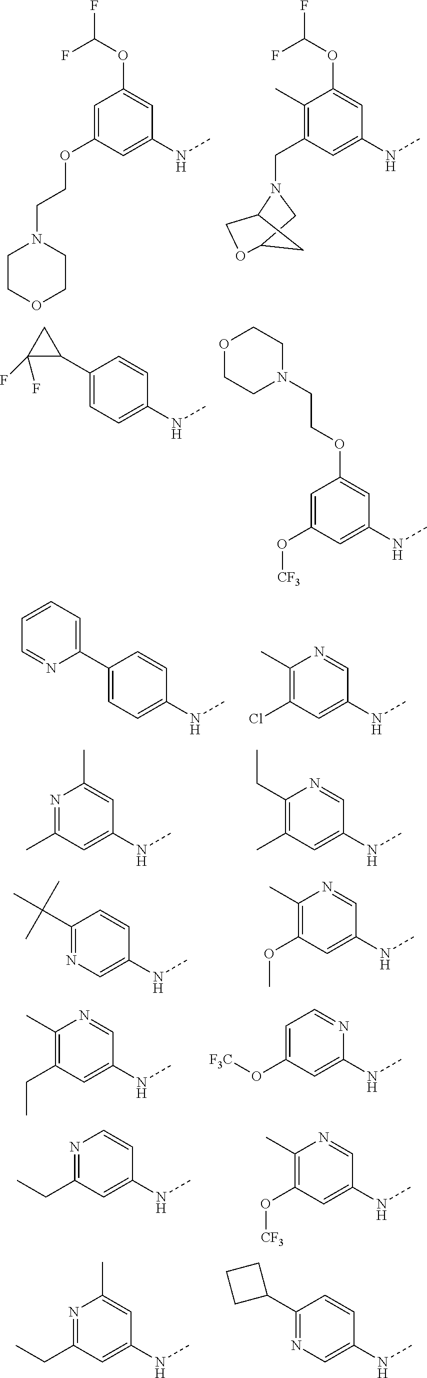

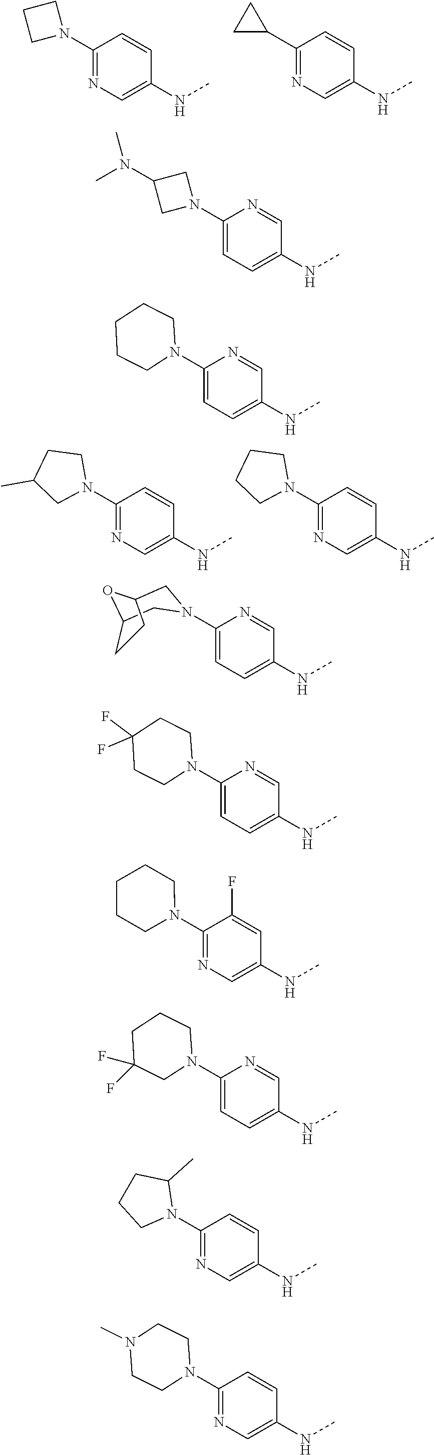

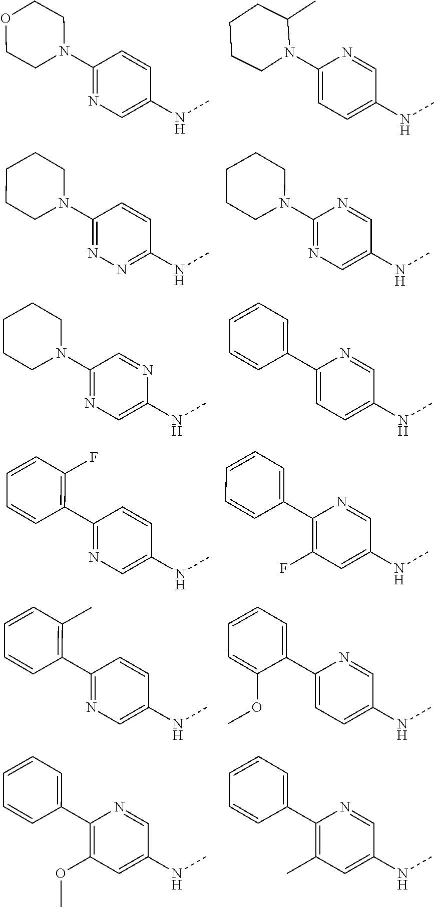

In some embodiments, the disclosure is directed to the (S) enantiomer of the compounds of any of formula I-XII. In some embodiments, the disclosure is directed to the (R) enantiomer of the compounds of any of formula I-XII. In some embodiments, the disclosure is directed to the racemate of the compounds of any of formula I-XII.

It is understood that the compounds of the disclosure may contain one or more asymmetric centers in the molecule. A compound without designation of the stereochemistry is to be understood to include all the optical isomers (e.g., diastereomers, enantiomers, etc.) in pure or substantially pure form, as well as mixtures thereof (e.g. a racemic mixture, or an enantiomerically enriched mixture). It is well known in the art how to prepare such optically active forms (e.g. by resolution of the racemic form by recrystallization techniques, by synthesis from optically-active starting materials, by chiral synthesis, by chromatographic separation using a chiral stationary phase, and other methods).

The compounds can be isotopically-labeled compounds, for example, compounds including various isotopes of hydrogen, carbon, nitrogen, oxygen, phosphorous, fluorine, iodine, or chlorine. The disclosed compounds may exist in tautomeric forms and mixtures and separate individual tautomers are contemplated. In addition, some compounds may exhibit polymorphism.

The compounds of the disclosure include the free form as well as the pharmaceutically acceptable salts and stereoisomers thereof. The pharmaceutically acceptable salts include all pharmaceutically acceptable salts known in the art and commonly used. The pharmaceutically acceptable salts of the present compounds can be synthesized from the compounds of this disclosure which contain a basic or acidic moiety by conventional chemical methods, see e.g. Berge et al, “Pharmaceutical Salts,” J. Pharm. ScL, 1977:66:1-19. Furthermore, the compounds of the disclosure also include lyophilized and polymorphs of the free form.

For example, conventional pharmaceutically acceptable salts for a basic compound include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Conventional pharmaceutically acceptable salts for an acidic compound include those derived from inorganic bases include aluminum, ammonium, calcium, copper, ferric, ferrous, lithium, magnesium, manganic salts, manganous, potassium, sodium, zinc and the like. Salts derived from pharmaceutically acceptable organic bases include salts of primary, secondary and tertiary amines, substituted amines including naturally occurring substituted amines, cyclic amines and basic ion exchange resins, such as arginine, betaine caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The compounds of the disclosure may exist in solid, i.e. crystalline or noncrystalline form (optionally as solvates) or liquid form. In the solid state, it may exist in, or as a mixture thereof. In crystalline solvates, solvent molecules are incorporated into the crystalline lattice during crystallization. The formation of solvates may include non-aqueous solvents such as, but not limited to, ethanol, isopropanol, DMSO, acetic acid, ethanolamine, or ethyl acetate, or aqueous solvents such as water (also called “hydrates”). It is common knowledge that crystalline forms (and solvates thereof) may exhibit polymorphism, i.e. exist in different crystalline structures known as “polymorphs”, that have the same chemical composition but differ in packing, geometrical arrangement, and other descriptive properties of the crystalline solid state. Polymorphs, therefore, may have different physical properties such as shape, density, hardness, deformability, stability, and dissolution properties, and may display different melting points, IR spectra, and X-ray powder diffraction patterns, which may be used for identification. Such different polymorphs may be produced, for example, by changing or adjusting the reaction conditions or reagents, during preparation of the compound of the disclosure.

In a further aspect, the disclosure also provides methods of preparation of the compounds of the disclosure. Typically, the compounds of the present disclosure may be prepared from readily available starting materials using the general methods and procedures disclosed in the Examples. It will be appreciated that the disclosed experimental conditions (e.g., reaction temperatures, time, moles of reagents, solvents, etc.) may vary with the particular reactants or solvents used, but such conditions can be determined by one skilled in the art by routine optimisation procedures.

In another aspect, the disclosure provides a pharmaceutical composition comprising a therapeutically-effective amount of one (or more) of the compounds of the disclosure or pharmaceutically acceptable salt thereof and one (or more) pharmaceutically acceptable carriers and/or excipients (also referred to as diluents) for use in a monotherapy in the treatment or the prevention of a disease or disorder caused by or associated with one or more premature termination codons. In some embodiments the pharmaceutical composition comprising a therapeutically-effective amount of one (or more) of the compounds of the disclosure or pharmaceutically acceptable salt thereof and one (or more) pharmaceutically acceptable carriers and/or excipients (also referred to as diluents) is used in a combined therapy with a therapeutically-effective amount of one (or more) of the aminoglycoside or pharmaceutically acceptable salt thereof in the treatment or the prevention of a disease or disorder caused by or associated with one or more premature termination codons.

For the combined therapy according to the disclosure a compound of formula I and an aminoglycoside of the disclosure may be in form of a separate pharmaceutical compositions (or dosage forms) each pharmaceutical composition (or dosage form) comprising one or more pharmaceutically acceptable excipient(s), which compositions are administered simultaneously or sequentially. Both pharmaceutical compositions (or dosage forms) provide a compound of formula I and an aminoglycoside in synergistically effective amounts for the treatment or the prevention of a disease or disorder caused by or associated with one or more premature termination codons.

The excipients for use in pharmaceutical compositions (or dosage forms) are acceptable in the sense of being compatible with the active agents of the disclosure and not deleterious to the recipient thereof (i.e., the patient). The term “therapeutically-effective amount” is defined as above and refers to the amount of a compound of the present disclosure and the amount of an aminoglycoside, which are effective for producing the desired synergistic therapeutic effect.

Pharmaceutical compositions may be in unit dosage forms containing a predetermined amount of a compound of the disclosure per unit dose and optionally a predetermined amount of an aminoglycoside of the disclosure per unit dose. Such a unit may contain a therapeutically effective dose of a compound of the disclosure or salt thereof and optionally a therapeutically effective dose of an aminoglycoside of the disclosure, or a fraction of one or both therapeutically effective doses such that multiple unit dosage forms might be administered at a given time to achieve the desired therapeutically effective dose. Preferred unit dosage forms are those containing a daily dose or sub-dose, or an appropriate fraction thereof, of each a compound of the disclosure or salt thereof and of an aminoglycoside of the disclosure.

The compounds of the disclosure may be administered by any acceptable means in solid or liquid form, including (1) oral administration, for example, drenches (aqueous or non-aqueous solutions or suspensions), tablets, capsules, e.g., those targeted for buccal, sublingual, and systemic absorption, boluses, powders, granules, syrups, pastes for application to the tongue; (2) parenteral administration, for example, by subcutaneous, intramuscular, intravenous or epidural injection as, for example, a sterile solution or a dispersion or suspension, or sustained-release formulation; (3) topical application, for example, as a cream, ointment, gel, emulsion or a controlled-release patch or spray applied to the skin; (4) intravaginally or intrarectally, for example, as a pessary, suppository, cream or foam; (5) sublingually; (6) ocularly; (7) transdermally; (8) nasally, for example, as a spray; (9) pulmonary; or (10) intrathecally.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical compositions.

Pharmaceutical compositions of the disclosure may contain further components conventional in pharmaceutical preparations, e.g. wetting agents, emulsifiers and lubricants, such as sodium lauryl sulfate and magnesium stearate, as well as coloring agents, release agents, coating agents, sweetening, flavoring and perfuming agents, preservatives and antioxidants, pH modifiers, bulking agents, and further active agents. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical compositions of the disclosure may be prepared by any method known in the art, for example, by bringing into association each of the active ingredients with one or more carriers and/or excipients. Different compositions and examples of carriers and/or excipients are well known to the skilled person and are described in detail in, e.g., Remington: The Science and Practice of Pharmacy. Pharmaceutical Press, 2013; Rowe, Sheskey, Quinn: Handbook of Pharmaceutical Excipients, Pharmaceutical Press, 2009. Excipients that may be used in the preparation of the pharmaceutical compositions of the disclosure may include one or more of buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide a composition suitable for an administration of choice.

In solid dosage forms of the disclosure for oral administration (capsules, tablets, pills, dragees, powders, granules, trouches and the like), each active agent of the present disclosure is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions of the disclosure may also comprise buffering agents. Solid compositions of a similar type may also be employed as fillers in soft and hard-shelled gelatin capsules using such excipients as lactose or milk sugars, as well as high molecular weight polyethylene glycols and the like. A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. Each of the active ingredients of the disclosure can also be in micro-encapsulated form, if appropriate, with one or more of the above-described excipients.

Liquid dosage forms for oral administration of one or both active agents of the disclosure include pharmaceutically acceptable emulsions, microemulsions, solutions, suspensions, syrups and elixirs. In addition to an active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. An oral composition can also include adjuvants such as wetting agents, emulsifying and suspending agents, sweetening, flavoring, coloring, perfuming and preservative agents.

In form of suspensions, an active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for rectal or vaginal administration of one or both active agents of the disclosure include a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Other suitable forms include pessaries, tampons, creams, gels, pastes, foams or spray formulations containing such carriers as are known in the art to be appropriate.

Dosage forms for the topical or transdermal administration of one or both active agents of the disclosure include powders, sprays, ointments, pastes, creams, lotions, gels, solutions, patches and inhalants. An active agent may be mixed under sterile conditions with a pharmaceutically-acceptable carrier, and with any preservatives, buffers, or propellants which may be required. Such ointments, pastes, creams and gels may contain, in addition to a compound of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Dosage forms such as powders and sprays for administration of one or both active agents of the disclosure, may contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Dosage forms such as transdermal patches for administration of one or both active agents of the disclosure may include absorption enhancers or retarders to increase or decrease the flux of the compound across the skin. The rate of such flux can be controlled by either providing a rate controlling membrane or dispersing the compound in a polymer matrix or gel. Other dosage forms contemplated include ophthalmic formulations, eye ointments, powders, solutions and the like. It is understood that all contemplated compositions must be stable under the conditions of manufacture and storage, and preserved against the contaminating action of microorganisms, such as bacteria and fungi.

The dosage levels of one or both active agents of the disclosure and the ratio of the total amounts of the active agents to be administered in the combined therapy may be adjusted in order to obtain an amount of each of the active agents of the disclosure which is effective to achieve the desired therapeutic response for a particular patient, composition, and mode of administration, without being deleterious to the patient. The dosage and ratio of choice will depend upon a variety of factors including the nature of the particular compound of the present disclosure and of the aminoglycoside used, the route of administration, the time of administration, the rate of excretion or metabolism of the particular compound used, the rate and extent of absorption, the duration of the treatment, other drugs, compounds and/or materials used in combination with the particular compound, the age, sex, weight, condition, general health and prior medical history of the patient being treated, and like factors well known in the medical arts. A medical practitioner having ordinary skill in the art can readily determine and prescribe the effective amount of the pharmaceutical compositions of the disclosure required.

Typically, a suitable (daily) dose of one or both active agents of the disclosure (and their ratio) will be that amount of one or both active agent, which is the lowest dose effective to produce the desired synergistic therapeutic effect, i.e. that the efficacy of the combined therapy is higher than the effect which would be obtained by use of only one of the active agents and may be determined according to standard methods. These suitable doses (and ratios) of a combined therapy to achieve a synergistic effect may be lower (than an individual administration).

A combined administration according to the disclosure may result not only in a beneficial effect, e.g., a synergistic therapeutic effect, e.g., with regard to alleviating, delaying progression of or inhibiting the symptoms, but also in further surprising beneficial effects, e.g., fewer side-effects, a more durable response, higher tolerability, an improved quality of life or a decreased morbidity, compared with an administration applying only one of the active agents of the invention. A further benefit is that lower doses of one or both of the active agents of a pharmaceutical combination as disclosed herein may be used (in particular if one (or both) of the agents is known to generate side effects), namely dosages, which may be non-effective in a use of a treatment with the individual active agents. A further benefit is that the active agents may need to be applied less frequently.

Generally, oral, intravenous, intracerebroventricular and subcutaneous doses of one or both active agents of this disclosure for a patient, when used for the indicated analgesic effects, will range from about 0.0001 to about 100 mg, more usual 0.1 to 100 mg/kg per kilogram of body weight of recipient (patient, mammal) per day. Acceptable daily dosages may be from about 1 to about 1000 mg/day, and for example, from about 1 to about 100 mg/day. Generally, the ratio of the compound of formula I to the aminoglycoside is between 100:1 to 1:100 (w/w), such as 50:1 to 1:50 (w/w), e.g. 10:1 to 1:10 (w/w).

The effective dose of one or both active agents of the disclosure may be administered as two, three, four, five, six or more sub-doses administered sequentially at appropriate intervals throughout a specified period (per day or per week or per month), e.g. in unit dosage forms. Preferred dosing also depends on factors as indicated above, e.g. on the administration route, particulars of the patient, nature and severity of the disease and the like and can be readily arrived at by one skilled in medicine or the pharmacy art.

The use or administration of a combined therapy (using a pharmaceutical combination of a compound of the disclosure and an aminoglycoside) as defined herein does not restrict the order or the timing (i.e. the time interval between administration of a compound of formula I and an aminoglycoside of the disclosure or pharmaceutical compositions of each) in which treatments or compounds are administered to a subject with a disease or disorder as defined herein. In a combined administration, the timing of an administration of the two (or more) active agents may be chosen such that an optimal synergistic therapeutic effect is achieved. Thus, in some embodiments the active agents of the disclosure may be administered (in form of pharmaceutical compositions or unit dosage forms) in a simultaneous or sequential manner. In some embodiments, a compound of formula I and an aminoglycoside are administered simultaneously. In some embodiments, a compound of formula I and an aminoglycoside are administered sequentially. The two or more active agents may be in form of one pharmaceutical composition or unit dosage form and thus are administered simultaneously. Simultaneous use or administration may also take place by simultaneously administering the two or more active agents in form of separate pharmaceutical compositions or unit dosage forms. Sequential use or administration refers to a use or administration of one (or more) compounds of the disclosure of a combined therapy at a first time point and one (or more) aminoglycosides of the disclosure at a second and different time point, such that the pharmaceutical combination shows a higher efficacy (i.e. a synergistic efficacy) than use or administration of only one of the active agents. The time interval between administration of each active agent, which allows to obtain the desired synergistic therapeutic effect, may be determined according to standard methods. For example, a first treatment (e.g. a compound of formula I as defined herein) may be administered prior to (e.g., 5 minutes, 15 minutes, 30 minutes, 45 minutes, 1 hour, 2 hours, 4 hours, 6 hours, 12 hours, 24 hours before), simultaneously with, or subsequent to (e.g., 5 minutes, 15 minutes, 30 minutes, 45 minutes, 1 hour, 2 hours, 4 hours, 6 hours, 12 hours, 24 hours after) the administration of a second treatment (e.g., an aminoglycoside compound as defined herein) to a subject in need thereof.

The compounds of the disclosure, optionally in combination with an aminoglycoside of the disclosure, are capable of a translational readthrough of a premature termination codon (i.e. achieving a nonsense suppression), which allows to restore functional protein expression and to reduce disease symptoms associated with the production of otherwise (partially) non-functional protein(s). Thus, the combined therapy of active agents of the disclosure or pharmaceutical compositions of the disclosure can be useful in the prevention and treatment of a disease or disorder caused by or associated with one or more premature termination codons, more specifically in the treatment of cancer.

Therefore, in a further aspect, the present disclosure provides a method of prevention and treatment of a disease or disorder caused by or associated with one or more premature termination codons (more specifically cancer) in a subject in need thereof, e.g. a human. In some embodiments, the use or method of treatment of a subject comprises administering to a subject in need of such treatment, e.g. a human, a therapeutically effective amount of a compound of formula I or a pharmaceutical composition thereof, wherein the composition further comprises one or more pharmaceutically acceptable vehicles, excipients or diluents as defined herein. In some embodiments, the use or method of treatment of a subject comprises administering to a subject in need of such treatment, e.g. a human, a therapeutically effective amount of a compound of formula I in combination with a therapeutically effective amount of an aminoglycoside or pharmaceutically acceptable salts thereof, or separate pharmaceutical compositions thereof, each composition further comprising one or more pharmaceutically acceptable vehicles, excipients or diluents as defined herein, which compositions may be administered, simultaneously or sequentially at defined time intervals as defined herein.

As used herein, the term “disorder” or “disease” or “pathologic condition” refers to a nonsense-mutation-mediated (or PTC-mediated) disorder or disease, i.e. a disorder or a disease associated with, characterized by or caused by premature translation termination and/or nonsense-mediated mRNA decay leading to inhibition of expression of protein having a normal function. In some embodiments, the genetic disease or disorder is a central nervous system disease, autoimmune disease, inflammatory disease, primary immunodeficiency disease, blood disease, DNA repair disorder, collagen disease, neuromuscular disease, cancer, etc.

In some embodiments, the genetic disease or disorder is selected from the group consisting of, but not limited to, beta-thalassemia, Ehlers-Danlos syndrome, severe myoclonic epilepsy of infancy, achromatopsia, retinitis pigmentosa, Usher Syndrome Type 1C, adducted thumb-clubfoot syndrome, Alagille syndrome, Alstroem syndrome, antithrombin deficiency, Carney complex, Currarino syndrome, Diamond-Blackfan anemia, erythropoietic protoporphyria, Fabry disease, factor XIII deficiency, Fanconi-Bickel syndrome, fish odor syndrome, Gaucher disease, hereditary hemorrhagic telangiectasia, homocystinuria, Joubert syndrome and related disorders, Krabbe disease, L-2-hydroxyglutaric aciduria, methylmalonic academia, Niemann-Pick disease, Peters plus syndrome, Townes-Brocks disease, von Willebrand disease, Wiskott-Aldrich syndrome, Kabuki syndrome, Chanarin-Dorfman syndrome, lecithin:cholesterol acyltransferase deficiency/fish-eye disease, Marfan Syndrome, mucopolysaccharidiosis, amyloidiosis, Late Infantile Neuronal Ceroid Lipofuscinosis, coenzyme Q10 Deficiency, peroxisome biogenesis disorders, lysosomal storage disorders, colorectal cancer, congenital enteropeptidase deficiency, cystic fibrosis, Hungarian Peutz-Jeghers Syndrome, Jervell and Lange-Nielsen syndrome, Lynch syndrome, microvillus inclusion disease, Peutz-Jeghers syndrome, xanthinuria, acidosis, Alport syndrome, Bardet-Biedl syndrome, Birt-Hogg-Dube syndrome, Dent's disease, Gitelman syndrome, hereditary leiomyomatosis and renal cell cancer, hereditary spherocytosis, leber congenital amaurosis, lysinuric protein intolerance, nephronophthisis, polycystic kidney disease, pseudohypoaldosteronism, renal hypodysplasia, sporadic clear cell renal cell carcinoma, type 2 papillary renal cell cancers, urofacial syndrome, von Hippel-Lindau disease, Wilms' tumor, X-linked Alport syndrome, X-linked hypophosphatemic rickets, hyperuricaemic nephropathy (juvenile/medullary cystic kidney disease), tuberous sclerosis, nephrotic syndrome/congenital nephrotic syndrome, Finnish type nephrotic syndrome, steroid resistant nephrotic syndrome 3, early onset nephrotic syndrome/Pierson syndrome, Denys-Drash syndrome, nephrotic syndrome/Schimke immuno-osseous dysplasia, primary glucocorticoid resistance, X-linked hypophosphatemia, primary hyperoxaluria type 1, pseudohypoaldosteronism type 1, proximal renal tubular acidosis, abetalipoproteinemia and homozygous familial hypobetalipoproteinemia, Alpers syndrome, carbamyl phosphate synthetase I deficiency, cholesteryl ester storage disease, citrin deficiency, Dubin-Johnson syndrome, erythropoietic protoporphyria, factor V deficiency, glycogen storage disease, Hemophilia A (factor VIII Deficiency), Hemophilia B (factor IX Deficiency), hepatocellular carcinoma, hepatoerythropoietic porphyria, hereditary spastic paraplegias, hypobetalipoproteinemia, inherited factor XI deficiency, diabetes mellitus (Type 1 and Type 2), microcytic anemia and iron deficiency, mitochondrial DNA depletion, mitochondrial DNA depletion syndrome, phenylketonuria, polycystic liver disease, porphyria cutanea tarda, progressive familial intrahepatic cholestasis, Wilson Disease, autosomal dominant hypercholesterolemia, factor XII deficiency, factor X deficiency, hypofibrinogenaemia, afibrinogenaemia, factor VII deficiency, agammaglobulinemia, amegakaryocytic thrombocytopenia, dyserythropoietic anemia type II, Duchenne and Becker muscular dystrophy, centronuclear myopathies, limb girdle muscular dystrophy or Miyoshi myopathy, Ullrich disease, spinal muscular atrophy, dystrophic epidermolysis bullosa, Hailey-Hailey Disease, Herlitz junctional epidermolysis bullosa, Netherton syndrome, ataxia-telangiectasia, Dravet syndrome, myotonic dystrophy, infantile neuronal ceroid lipofuscinosis, Alzheimer's disease, Tay-Sachs disease, neural tissue degeneration, Parkinson's disease, lupus erythematosus, graft-versus-host disease, severe combined immunodeficiency, DNA Ligase IV deficiency, Nijmegen breakage disorders, xeroderma pigmentosum (XP), familial erythrocytosis, nephrolithiasis, osteogenesis imperfect, cirrhosis, neurofibroma, bullous disease, lysosomal storage disease, Hurler's disease, familial cholesterolemia; cerebellar ataxia; lung disease; cystic fibrosis; pigmentary retinopathy; amyloidosis, atherosclerosis, gigantism, dwarfism, hypothyroidism, hyperthyroidism, obesity; and any other genetic disorder caused by nonsense mutation(s).

The term “cancer” when associated with, characterized by or caused by premature translation termination and/or nonsense-mediated mRNA decay, e.g. cancer associated with a nonsense mutation of a suppressor gene such as the p53 gene, includes all types of cancer, for example lung cancer, colon and rectal cancer, stomach cancer, esophageal cancer, kidney cancer, pancreatic cancer, prostate cancer, breast cancer, uterine cancer, ovarian cancer, skin cancer, sarcoid, leukemia, lymphoma, and brain tumor.

The present disclosure further contemplates administration of a compound of the disclosure in a combined therapy with an aminoglycoside of the disclosure and further in combination with one or more additional therapeutic agents. The pharmaceutical combination for use according to the disclosure may also be combined with other treatments or therapies, e.g., surgical intervention, irradiation therapies, chemotherapies, radiotherapies, hormonal therapies, if suitable for added clinical effectiveness.

The active agents of a combined therapy may further be in form of a kit together with instructions for simultaneous or sequential use thereof in the prevention or treatment of a disorder or disease as defined herein.

In the following the present disclosure shall be illustrated by means of some examples which are not construed to be viewed as limiting the scope of the disclosure.

### EXAMPLES

### General Synthetic Procedures

### General Procedure A:

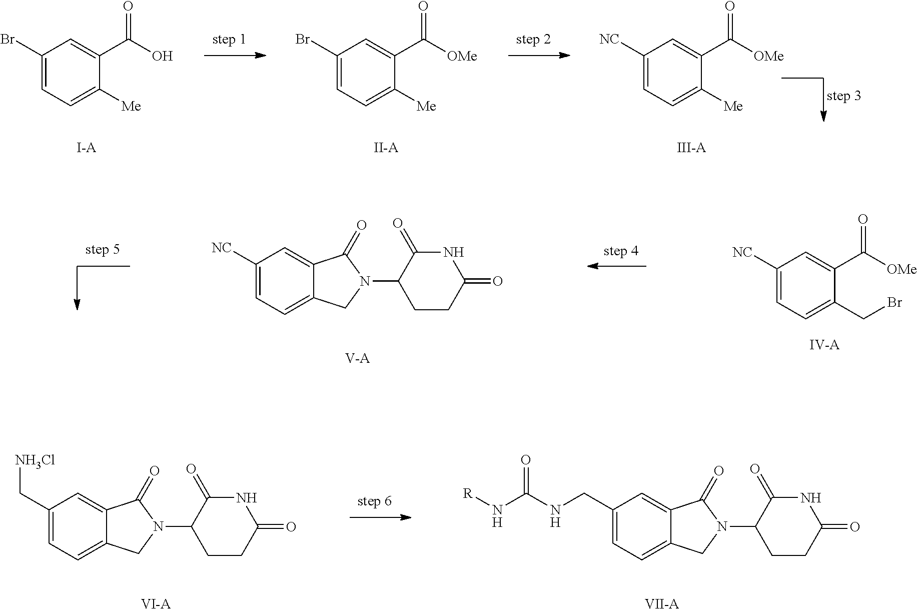

II-A: To a solution of 5-bromo-2-methyl-benzoic acid (100 g, 465 mmol, 1.00 eq) in methanol (700 mL) was added sulfuric acid (31.3 g, 313 mmol, 0.672 eq) dropwise at 0° C. The mixture was stirred at 25° C. for 1 h and then heat to 70° C. for 12 h. The mixture was cooled to 25° C. and concentrated under reduced pressure. The residue was poured into ice-water (200 mL) and basified with solid sodium carbonate to pH=8. The aqueous phase was extracted with ethyl acetate (3×300 mL). The combined organic phase was washed with brine (2×100 mL), dried over anhydrous sodium sulfate, filtered and concentrated in vacuum to afford methyl 5-bromo-2-methyl-benzoate II.

III-A: To a solution of methyl 5-bromo-2-methyl-benzoate II-A (113 g, 493 mmol, 1.00 eq) in N,N-dimethylformamide (300 mL) was added copper(I)cyanide (66.3 g, 1.50 eq). The mixture was stirred at 150° C. for 12 h. The mixture was quenched by ice slowly and then extracted with ethyl acetate (2×500 mL). The aqueous phase was extracted with ethyl acetate (2×300 mL). The combined organic phase was washed with brine (2×100 mL), dried over anhydrous sodium sulfate, filtered and concentrated in vacuum. The residue was purified by silica gel column chromatography (petroleum ether/ethyl acetate=0/1 to 100/1) to afford methyl 5-cyano-2-methyl-benzoate III-A. ¹H NMR (400 MHz, CDCl₃-d) δ=8.22 (d, J=1.5 Hz, 1H), 7.67 (dd, J=1.7, 7.9 Hz, 1H), 7.38 (d, J=7.9 Hz, 1H), 3.93 (s, 3H), 2.68 (s, 3H).

IV-A: To a solution of methyl 5-cyano-2-methyl-benzoate III-A (61.0 g, 348 mmol, 1.00 eq) and N-bromosuccinimide (75.8 g, 383 mmol, 1.10 eq) in carbon tetrachloride (650 mL) was added benzoyl peroxide (8.43 g, 34.8 mmol, 0.100 eq). The mixture was stirred at 80° C. for 12 h. The mixture was concentrated in vacuum, suspended in water (500 mL) and then extracted with ethyl acetate (2×500 mL). The aqueous phase was extracted with ethyl acetate (2×300 mL). The combined organic phase was washed with brine (2×100 mL), dried over anhydrous sodium sulfate, filtered and concentrated in vacuum. The residue was purified by silica gel chromatography (petroleum ether/ethyl acetate=0/1 to 5/1) to afford methyl 2-(bromomethyl)-5-cyano-benzoate IV-A. ¹H NMR (400 MHz, CDCl₃-d) δ=8.20 (d, J=1.6 Hz, 1H), 7.70 (dd, J=1.8, 8.0 Hz, 1H), 7.54 (d, J=8.0 Hz, 1H), 4.89 (s, 2H), 3.91 (s, 3H).

V-A: To a solution of methyl 2-(bromomethyl)-5-cyano-benzoate IV-A (40 g, 157 mmol, 1.00 eq) and 3-aminopiperidine-2,6-dione (25.9 g, 157 mmol, 1.00 eq, hydrochloric acid) in dimethylsulfoxide (200 mL) was added triethylamine (65.7 mL, 3.00 eq). The mixture was stirred at 100° C. for 2 h. The crude product was triturated with water (300 mL). The mixture was filtered, and the filtrate was concentrated in vacuum, and then the solid was triturated with methanol (30.0 mL). The residue was filtered, and the filtrate was concentrated in vacuum to afford 2-(2,6-dioxo-3-piperidyl)-3-oxo-isoindoline-5-carbonitrile V-A. ¹H NMR (400 MHz, DMSO-d6) δ=11.01 (s, 1H), 8.20 (s, 1H), 8.09 (dd, J=1.5, 7.9 Hz, 1H), 7.85 (d, J=7.9 Hz, 1H), 5.15 (dd, J=5.1, 13.3 Hz, 1H), 4.64-4.40 (m, 2H), 2.99-2.85 (m, 1H), 2.65-2.56 (m, 1H), 2.44-2.34 (m, 1H), 2.03 (dtd, J=2.0, 5.2, 12.5 Hz, 1H).

VI-A: To a solution of 2-(2,6-dioxo-3-piperidyl)-3-oxo-isoindoline-5-carbonitrile V-A (5.00 g, 18.6 mmol, 1.00 eq) and hydrochloric acid (12 M, 5.00 mL, 3.23 eq) in methanol (500 mL) was added platinum(IV)oxide (1.05 g, 4.64 mmol, 0.250 eq) under nitrogen. The suspension was degassed under vacuum and purged with hydrogen several times. The mixture was stirred under hydrogen (15 psi) at 25° C. for 12 h. The reaction mixture was filtered, and the filtrate was concentrated in vacuum. The crude product was triturated with ethyl alcohol (10.0 mL), the mixture was filtered and the filtrate was dried in vacuum to afford 3-(6-(aminomethyl)-1-oxoisoindolin-2-yl)piperidine-2,6-dione hydrochloride VI-A. ¹H NMR (400 MHz, DMSO-d6) δ=10.99 (s, 1H), 8.54 (br s, 3H), 7.89 (d, J=0.7 Hz, 1H), 7.75 (dd, J=1.6, 7.8 Hz, 1H), 7.65 (d, J=7.8 Hz, 1H), 5.12 (dd, J=5.1, 13.3 Hz, 1H), 4.54 -4.30 (m, 2H), 4.14 (q, J=5.3 Hz, 2H), 2.97-2.85 (m, 1H), 2.65-2.57 (m, 1H), 2.44-2.38 (m, 1H), 2.01 (dtd, J=2.0, 5.2, 12.5 Hz, 1H).

### VII-A:

Variant i): To a solution of 3-(6-(aminomethyl)-1-oxoisoindolin-2-yl)piperidine-2,6-dione hydrochloride VI-A (1.00 eq, hydrochloride) and triethylamine (3.0 eq) in N,N-dimethylformamide (0.15 M) was added the isocyanate R—NCO (1 eq) in N,N-dimethylformamide (0.5 M) at 0° C. The mixture was stirred at rt for 45 min. Polymer supported trisamine (2.0 eq) was added and stirred for 30 min. The mixture was filtered, washed with N,N-dimethylformamide, and the filtrate concentrated in vacuum. The residue was purified by standard methods to afford the final urea-compounds VII-A.

Variant ii): To a solution of 3-(6-(aminomethyl)-1-oxoisoindolin-2-yl)piperidine-2,6-dione hydrochloride VI-A (1.00 eq, hydrochloride) and triethylamine (1.10 eq) in N,N-dimethylformamide (0.1 M) was added the isocyanate R—NCO (1.10 eq) at 0° C. The mixture was stirred at 25° C. for 1 h. The mixture was quenched by the addition of methanol and concentrated in vacuum. The residue was purified by standard methods and lyophilized to afford the final urea-compounds VII-A.

### General Procedure B

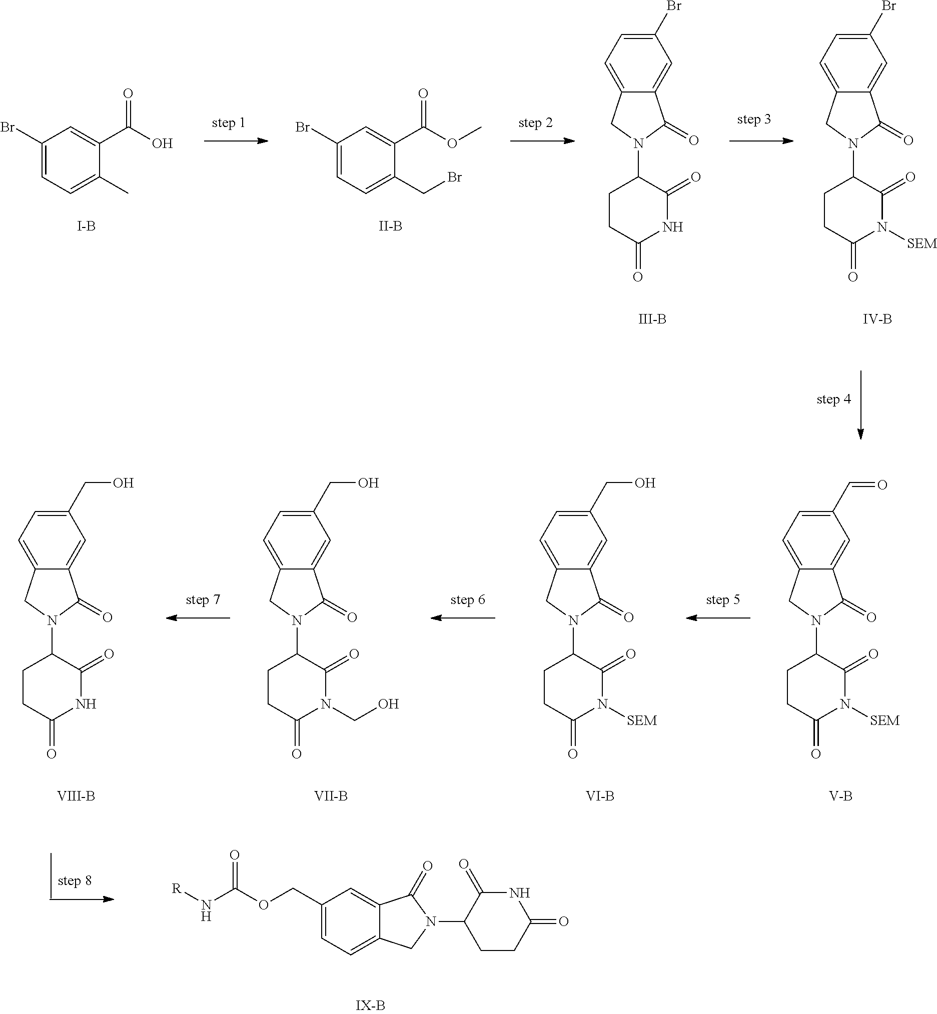

II-B: To a solution of methyl 5-bromo-2-methylbenzoate I (100 g, 436 mmol, 1.00 eq) in trichloromethane (800 mL) was added N-bromosuccinimide (77.6 g, 436 mmol, 1.00 eq) and (E)-2,2′-(diazene-1,2-diyl)bis(2-methylpropanenitrile) (10.5 g, 43.3 mmol, 0.10 eq). The solution was degassed by purging with nitrogen, and the reaction was stirred at 80° C. for 12 h under nitrogen atmosphere. The mixture was concentrated under reduced pressure to give a residue. The residue was purified by silica gel column chromatography (petroleum ether/ethyl acetate=1/0 to 20/1) to afford methyl 5-bromo-2-(bromomethyl)benzoate II-B. ¹H NMR (400 MHz, DMSO-d₆) δ=7.98 (d, J=2.0 Hz, 1H), 7.81-7.79 (m, 1H), 7.57-7.55 (m, 1H), 4.97 (s, 2H), 3.87 (m, 3H).

III-B: To a solution of methyl 5-bromo-2-(bromomethyl)benzoate II-B (72.5 g, 235 mmol 1.00 eq) and 3-aminopiperidine-2,6-dione hydrochloride (46.6 g, 283 mmol, 1.20 eq, hydrochloride) in acetonitrile (600 mL) was added diisopropylethylamine (123 mL, 706 mmol, 3.00 eq) in one portion under nitrogen. The reaction was stirred at 80° C. for 4 h. After cooling to room temperature, the mixture was concentrated under reduced pressure to give a residue. The residue was triturated with hydrochloric acid (1 M)/ethyl acetate (300 mL/200 mL) at 25° C. for 30 min. The mixture was filtered, and the filter cake was washed with ethyl acetate (100 mL) and dried under reduced pressure to afford 3-(6-bromo-1-oxoisoindolin-2-yl)piperidine-2,6-dione III-B. ¹H NMR (400 MHz, DMSO-d₆) δ=11.04 (s, 1H), 7.86 (d, J=1.6 Hz, 1H), 7.82 (dd, J=1.8, 8.1 Hz, 1H), 7.60 (d, J=8.1 Hz, 1H), 5.12 (dd, J=5.1, 13.3 Hz, 1H), 4.49-4.28 (m, 2H), 2.99-2.84 (m, 1H), 2.60 (br d, J=17.5 Hz, 1H), 2.39 (dq, J=4.4, 13.2 Hz, 1H), 2.13-1.91 (m, 1H).

IV-B: To a solution of 3-(6-bromo-1-oxoisoindolin-2-yl)piperidine-2,6-dione III-B (40.0 g, 123 mmol, 1.00 eq) in dimethylformamide (200 mL) was added 1,8-Diazabicyclo[5.4.0]-7-undecene (48.0 mL, 318 mmol, 2.57 eq) and 2-(chloromethoxy)ethyltrimethylsilane (stabilized with diisopropylethylamine) (40.0 mL, 226 mmol, 1.83 eq) dropwise at 0° C. The reaction was stirred at 25° C. for 6 h. The mixture was diluted with water (500 mL) and ethyl acetate (800 mL). The organic phase was washed with brine (200 mL), dried over anhydrous sodium sulfate, filtered, and concentrated under reduced pressure to give a residue. The residue was purified by silica gel column chromatography (petroleum ether/ethyl acetate=4/1 to 1/1) to afford 3-(6-bromo-1-oxoisoindolin-2-yl)-1-((2-(trimethylsilyl)ethoxy)methyl)piperidine-2,6-dione IV-B. ¹H NMR (400 MHz, DMSO-d₆) δ=7.90 (d, J=1.6 Hz, 1H), 7.86 (dd, J=2.0, 8.1 Hz, 1H), 7.63 (d, J=8.1 Hz, 1H), 5.26 (dd, J=5.1, 13.4 Hz, 1H), 5.07 (q, J=9.8 Hz, 2H), 4.50 (d, J=17.6 Hz, 1H), 4.36-4.28 (m, 1H), 3.60-3.49 (m, 2H), 3.13-3.01 (m, 1H), 2.86-2.78 (m, 1H), 2.48-2.36 (m, 1H), 2.12-2.05 (m, 1H), 0.90-0.83 (m, 2H), 0.02-0.01 (m, 9H).

V-B: To a solution of 3-(6-bromo-1-oxoisoindolin-2-yl)-1-((2-(trimethylsilyl)ethoxy)methyl)piperidine-2,6-dione IV-B (53.0 g, 117 mmol, 1 eq) in dimethylformamide (300 mL) was added diisopropylethylamine (100 mL, 574 mmol, 4.91 eq), [1,1′-bis(diphenylphosphino)ferrocene]dichloropalladium(II) (8.48 g, 11.6 mmol, 0.10 eq) and triethylsilane (150 mL, 939 mmol, 8.00 eq). The reaction was stirred at 80° C. for 12 h under carbon monoxide atmosphere (50 Psi). The mixture was diluted with ethyl acetate (1.00 L) and water (1.00 L). The organic layer was separated and concentrated under reduced pressure to give a residue. The residue was purified by silica gel column chromatography (petroleum ether/ethyl acetate=1/1) to afford 2-(2,6-dioxo-1-((2-(trimethylsilyl)ethoxy)methyl)piperidin-3-yl)-3-oxoisoindoline-5-carbaldehyde V. ¹H NMR (400 MHz, DMSO-d₆) δ=10.16 (s, 1H), 8.29 (s, 1H), 8.19 (d, J=7.8 Hz, 1H), 7.87 (d, J=7.8 Hz, 1H), 5.30 (br dd, J=4.9, 13.4 Hz, 1H), 5.13-5.03 (m, 2H), 4.65 (br d, J=18.4 Hz, 1H), 4.50-4.40 (m, 1H), 3.62-3.51 (m, 2H), 3.17-3.06 (m, 1H), 2.83 (br d, J=15.8 Hz, 1H), 2.44 (br dd, J=4.1, 13.1 Hz, 1H), 2.15-2.07 (m, 1H), 0.86 (br t, J=7.6 Hz, 2H), 0.00 (s, 9H)

VI-B: To a solution of 2-(2,6-dioxo-1-((2-(trimethylsilyl)ethoxy)methyl)piperidin-3-yl)-3-oxoisoindoline-5-carbaldehyde V-B (38.0 g, 94.4 mmol, 1.00 eq) in dimethylformamide (100 mL) and dichloromethane (500 mL) was added sodium triacetoxyborohydride (64.0 g, 302 mmol, 3.20 eq) and acetic acid (27.9 g, 465 mmol, 4.93 eq). The reaction was stirred at 50° C. for 2 h. The mixture was diluted with water (500 mL) and dichloromethane (500 mL). The organic layer was separated and concentrated under reduced pressure to give a residue. The residue was purified by silica gel column chromatography (petroleum ether/ethyl acetate=5/1 to 0/1) to give 3-(6-(hydroxymethyl)-1-oxoisoindolin-2-yl)-1-((2-(trimethylsilyl)ethoxy)methyl) piperidine-2,6-dione VI-B. ¹H NMR (400 MHz, DMSO-d₆) δ=7.70 (s, 1H), 7.62-7.52 (m, 2H), 5.35 (t, J=5.8 Hz, 1H), 5.29-5.20 (m, 1H), 5.06 (q, J=9.8 Hz, 2H), 4.61 (d, J=5.8 Hz, 2H), 4.47 (br d, J=17.1 Hz, 1H), 4.28 (d, J=16.9 Hz, 1H), 3.61-3.49 (m, 2H), 3.11-3.02 (m, 1H), 2.84-2.77 (m, 1H), 2.40 (br d, J=8.7 Hz, 1H), 2.11-2.03 (m, 1H), 0.89-0.81 (m, 2H), 0.01-0.03 (m, 9H).

VII-B: A solution of 3-(6-(hydroxymethyl)-1-oxoisoindolin-2-yl)-1-((2-(trimethylsilyl)ethoxy)methyl)piperidine-2,6-dione VI-B (17.0 g, 42.0 mmol, 1.00 eq) in hydrochloric acid/dioxane (150 mL) (6 M) was stirred at 50° C. for 2 h. The mixture was concentrated under reduced pressure to afford 1-(hydroxymethyl)-3-(6-(hydroxymethyl)-1-oxoisoindolin-2-yl)piperidine-2,6-dione VII-B.

VIII-B: To a solution of 1-(hydroxymethyl)-3-(6-(hydroxymethyl)-1-oxoisoindolin-2-yl)piperidine-2,6-dione VII-B (13.0 g, 42.7 mmol, 1 eq) in acetonitrile (50.0 mL) was added ammonium hydroxide 30% (0.500 mL, 0.09 eq). The reaction was stirred at 25° C. for 1 h. The pH was adjusted to pH=5 by addition of 0.5 M hydrochloric acid, and the mixture was concentrated under reduced pressure to give a residue. The residue was purified by reversed phase preparative HPLC to afford 3-(6-(hydroxymethyl)-1-oxoisoindolin-2-yl)piperidine-2,6-dione VIII-B. ¹H NMR (400 MHz, DMSO-d₆) δ=11.24-10.78 (m, 1H), 7.69 (s, 1H), 7.60-7.53 (m, 2H), 5.35 (t, J=5.8 Hz, 1H), 5.12 (dd, J=5.1, 13.4 Hz, 1H), 4.61 (d, J=5.5 Hz, 2H), 4.49-4.40 (m, 1H), 4.35-4.26 (m, 1H), 2.98-2.86 (m, 1H), 2.66-2.57 (m, 1H), 2.40 (dd, J=4.4, 13.1 Hz, 1H), 2.01 (dtd, J=2.2, 5.2, 12.6 Hz, 1H).

IX-B: Variant i): To a solution of 3-(6-(hydroxymethyl)-1-oxoisoindolin-2-yl)piperidine-2,6-dione VIII-B (1.00 eq) in dimethylformamide or acetonitrile (0.04-0.73 M reaction) was added the phenyl carbamate (0.66-1.70 eq) and sodium hydride (60% dispersion in mineral oil) (1.70-3.00 eq) at 0° C. The reaction was stirred at a temperature range of 0 to 25° C. for 0.5-4 h. If necessary, upon completion the reaction was acidified with HCl. If the product precipitated, it was collected by filtration to afford the final carbamate compounds as white solids. Otherwise, the mixture was either concentrated under reduced pressure to give a residue, or it was extracted (ethyl acetate/water) and the organic layers were combined, dried over sodium sulfate, filtered, and concentrated to give a residue. The residue was purified by a standard method to afford the final carbamate compounds.

IX-B: Variant ii): To a solution of 3-(6-(hydroxymethyl)-1-oxoisoindolin-2-yl)piperidine-2,6-dione VIII-B (1.50 g, 5.47 mmol, 1.00 eq) in dimethylformamide (15.0 mL) was added pyridine (2.21 mL, 27.3 mmol, 5.00 eq) and phenyl chloroformate (1.37 mL, 10.9 mmol, 2.00 eq) at 0° C. The reaction was stirred at 25° C. for 12 h. The mixture was concentrated under reduced pressure to give a residue. The residue was purified by reversed phase preparative HPLC to afford (2-(2,6-dioxopiperidin-3-yl)-3-oxoisoindolin-5-yl)methyl phenyl carbonate. To a solution of (2-(2,6-dioxopiperidin-3-yl)-3-oxoisoindolin-5-yl)methyl phenyl carbonate (1.00 eq) in dimethylformamide (0.1-0.14 M reaction) was added the amine (1.50-5.00 eq). The reaction was stirred at 25° C. for 1-36 h. The mixture was either: diluted to 0.07 M with dimethylformamide and purified by a standard method, or: diluted to 0.10 M with formic acid to give a precipitate which was filtered and dried, or: diluted to 0.10 M with formic acid and purified by a standard method, to afford the final carbamate compounds IX-B.

### Example 1: Preparation of Compounds 1 to 160 and 200-307

TABLE 1
Specific examples 1-160

| Compound | No. |
|---|---|
| 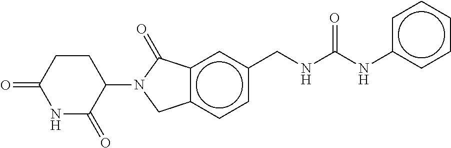 | 1 |
| 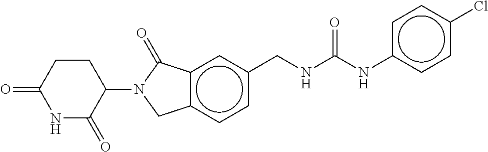 | 2 |
| 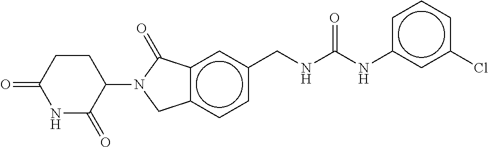 | 3 |
| 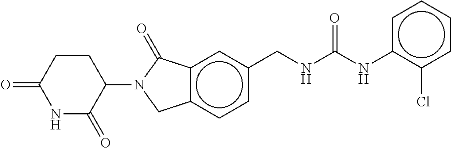 | 4 |
| 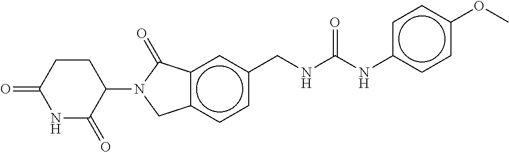 | 5 |
| 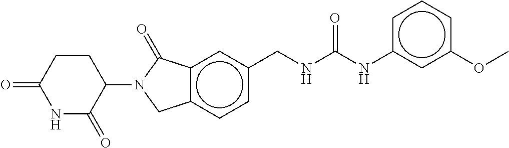 | 6 |
| 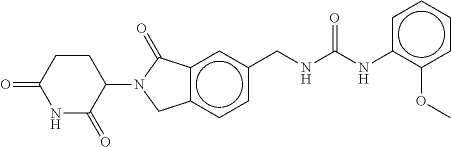 | 7 |
| 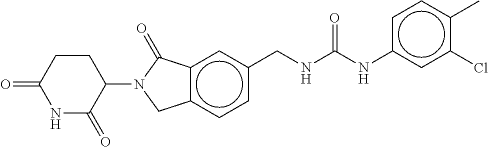 | 8 |
| 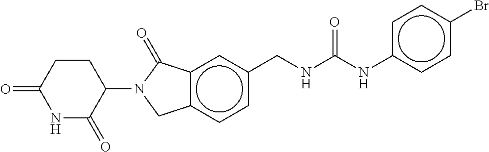 | 9 |
| 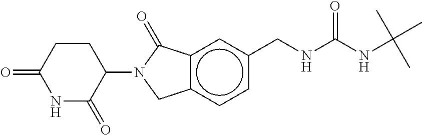 | 10 |
| 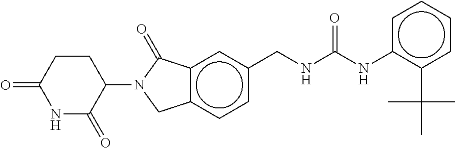 | 11 |
| 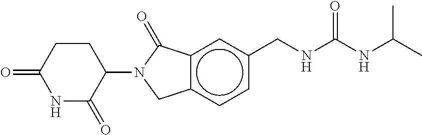 | 12 |
| 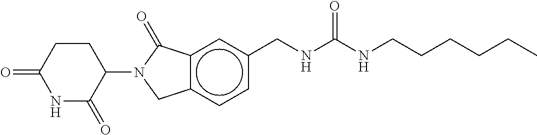 | 13 |
| 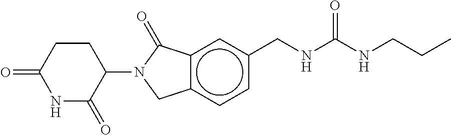 | 14 |
| 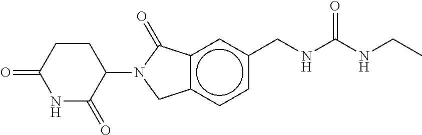 | 15 |
| 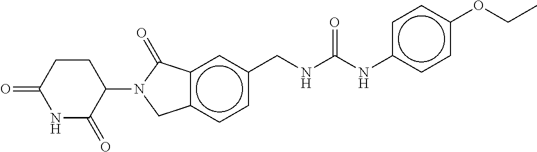 | 16 |
| 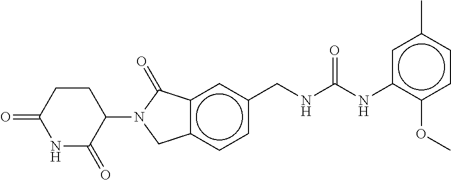 | 17 |
| 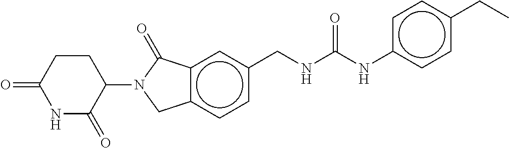 | 18 |
| 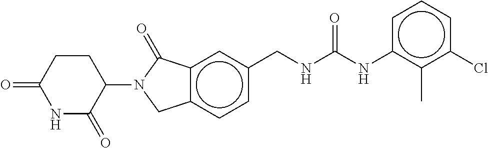 | 19 |
| 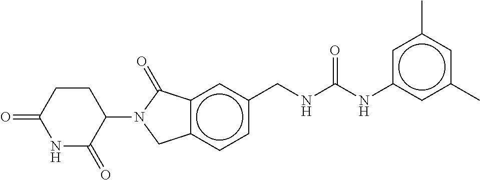 | 20 |
| 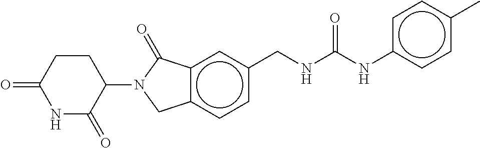 | 21 |
| 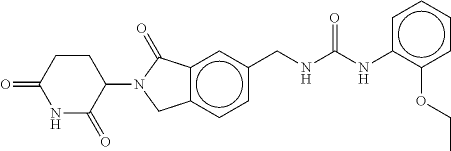 | 22 |
| 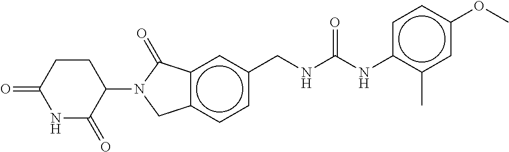 | 23 |
| 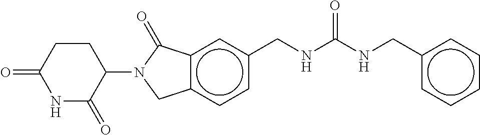 | 24 |
| 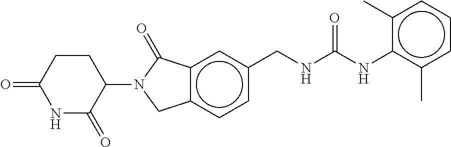 | 25 |
| 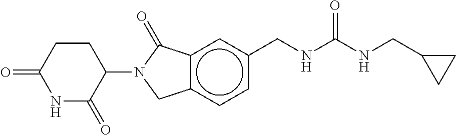 | 26 |
| 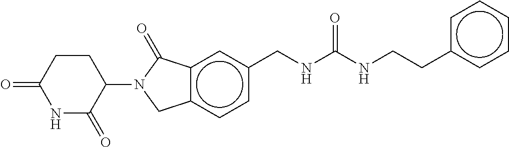 | 27 |
| 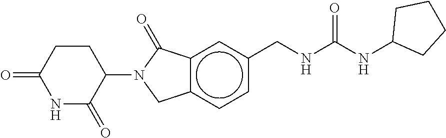 | 28 |
| 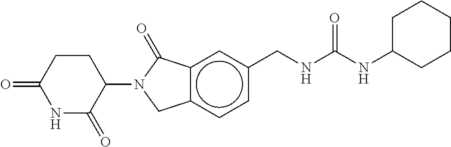 | 29 |
| 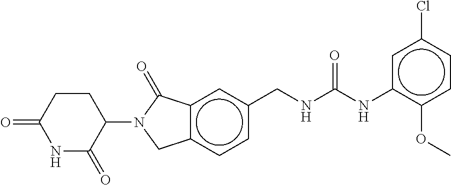 | 30 |
| 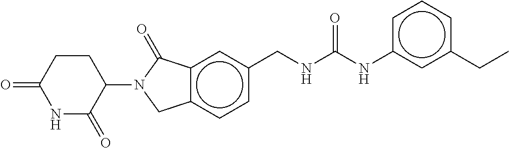 | 31 |
| 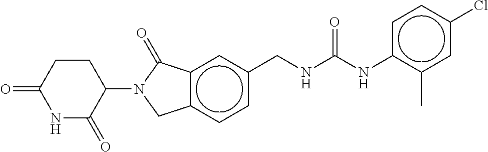 | 32 |
| 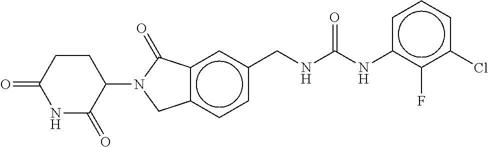 | 33 |
| 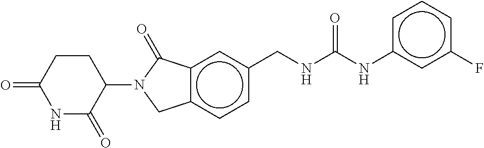 | 34 |
| 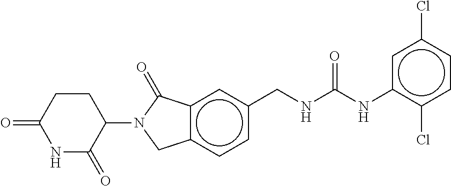 | 35 |
| 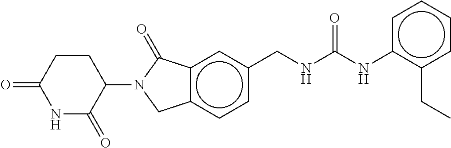 | 36 |
| 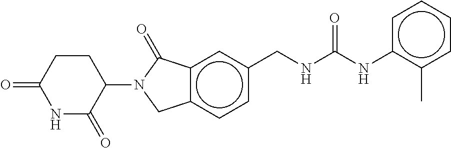 | 37 |
| 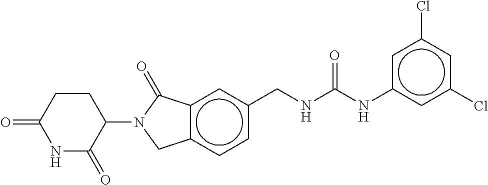 | 38 |
| 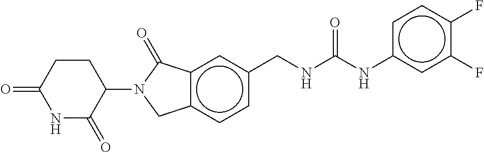 | 39 |
| 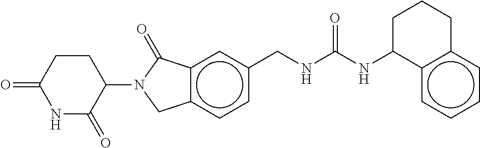 | 40 |
| 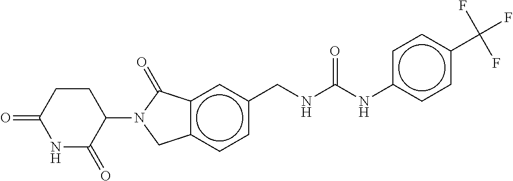 | 41 |
| 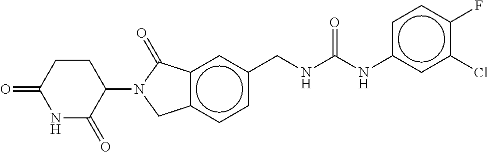 | 42 |
| 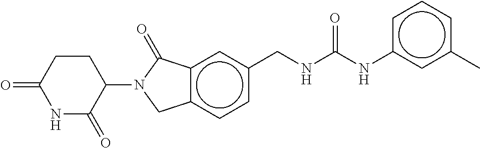 | 43 |
| 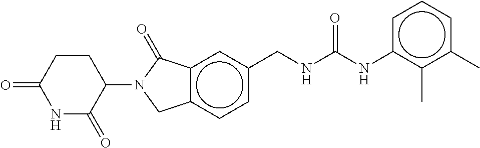 | 44 |
| 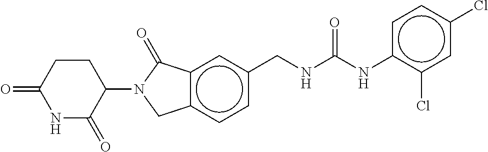 | 45 |
| 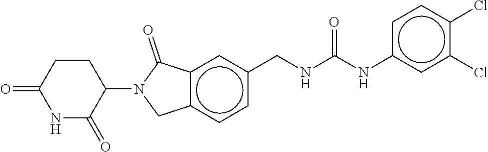 | 46 |
| 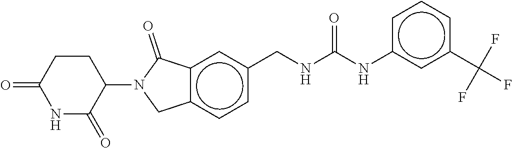 | 47 |
| 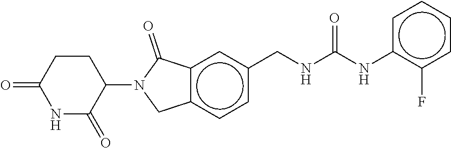 | 48 |
| 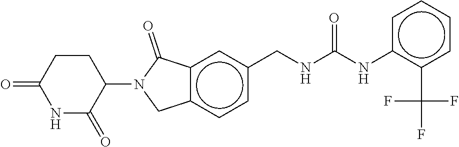 | 49 |
| 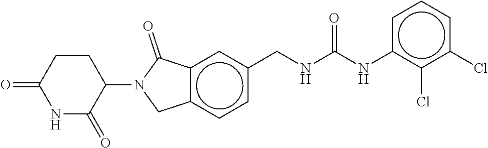 | 50 |
| 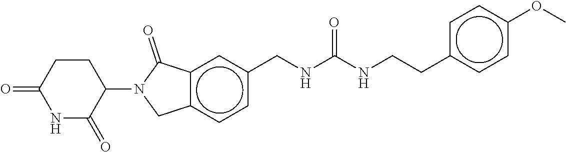 | 51 |
| 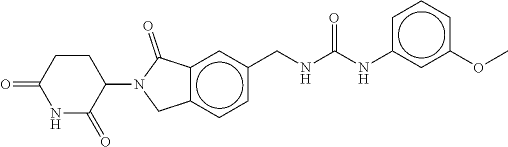 | 52 |
| 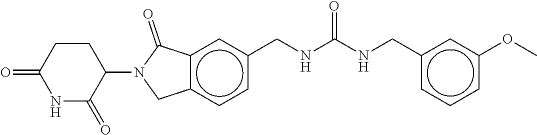 | 53 |
| 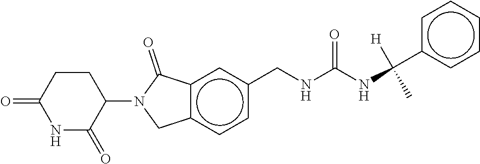 | 54 |
| 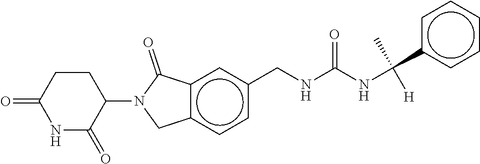 | 55 |
| 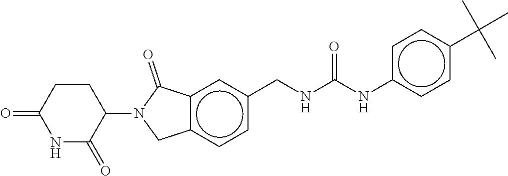 | 56 |
| 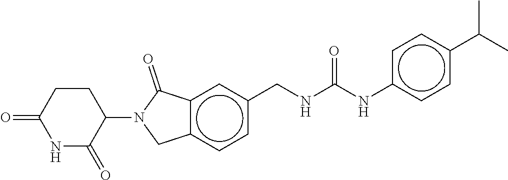 | 57 |
| 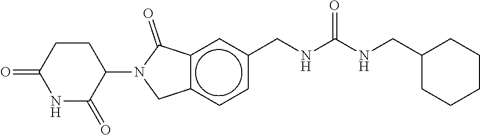 | 58 |
| 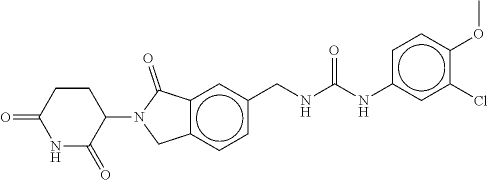 | 59 |
| 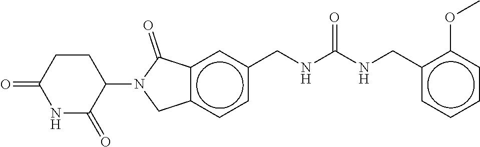 | 60 |
| 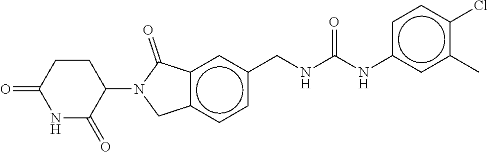 | 61 |
| 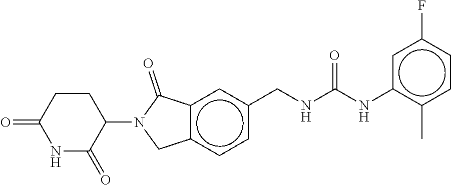 | 62 |
| 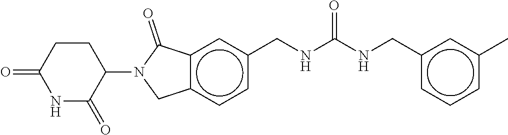 | 63 |
| 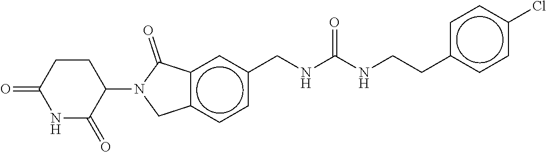 | 64 |
| 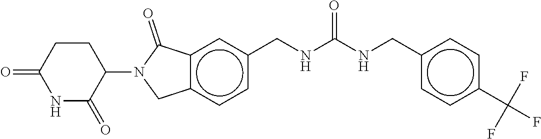 | 65 |
| 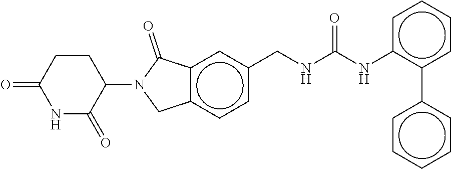 | 66 |
| 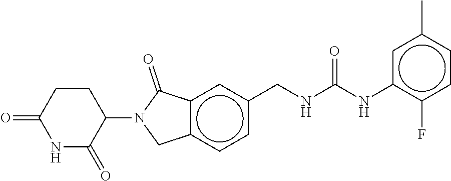 | 67 |
| 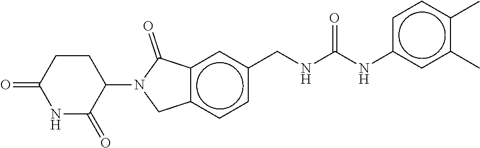 | 68 |
| 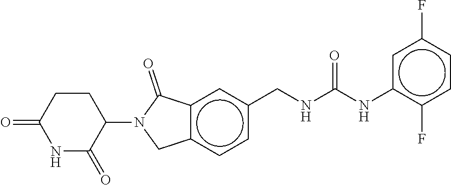 | 69 |
| 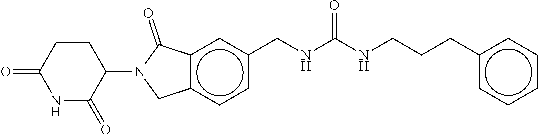 | 70 |
| 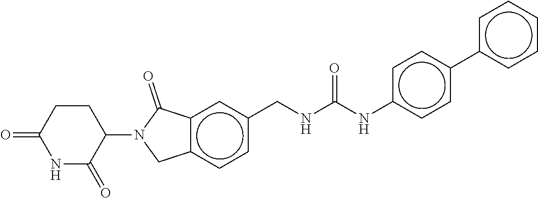 | 71 |
| 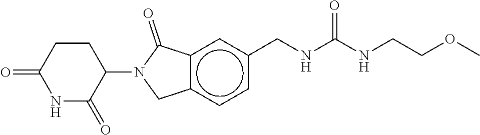 | 72 |
| 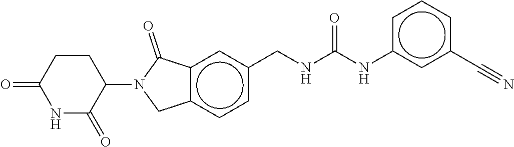 | 73 |
| 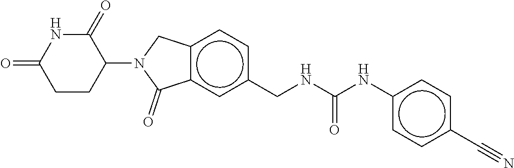 | 74 |
| 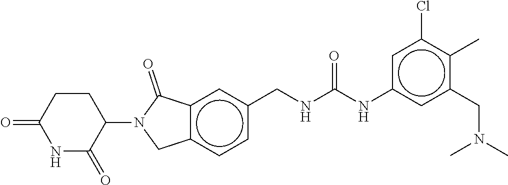 | 75 |
| 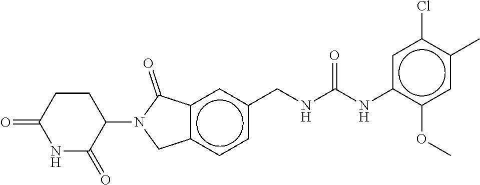 | 76 |
| 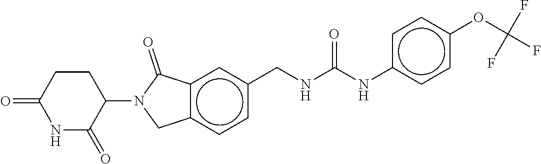 | 77 |
| 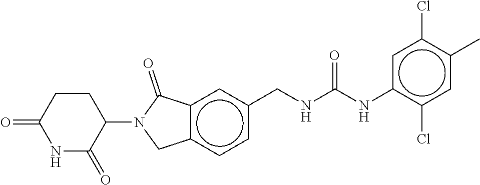 | 78 |
| 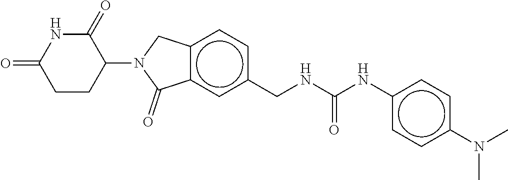 | 79 |
| 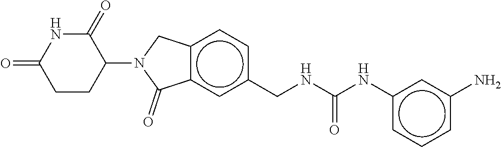 | 80 |
| 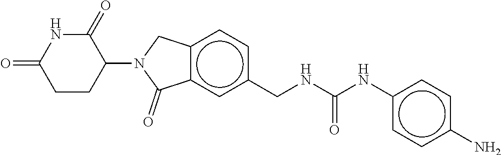 | 81 |
| 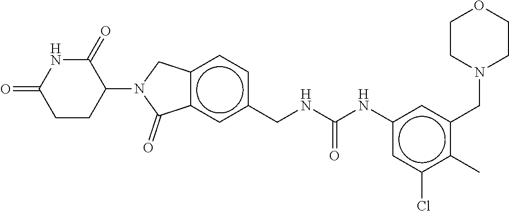 | 82 |
| 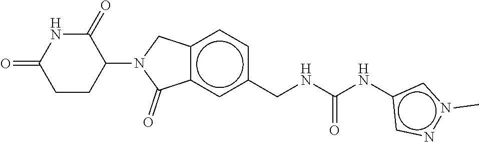 | 83 |
| 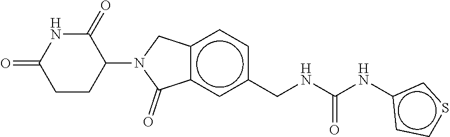 | 84 |
| 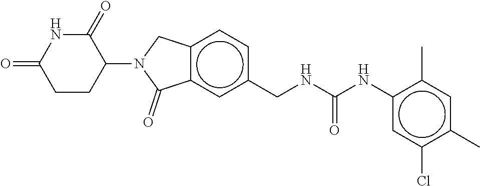 | 85 |
| 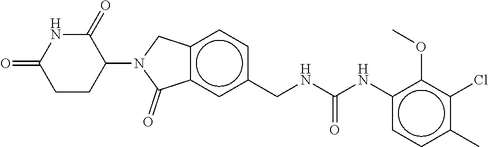 | 86 |
| 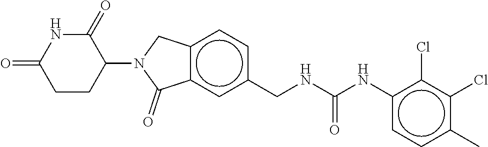 | 87 |
| 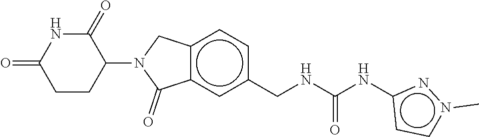 | 88 |
| 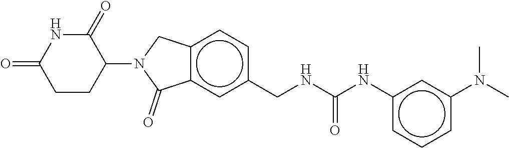 | 89 |
| 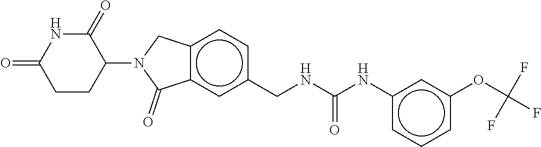 | 90 |
| 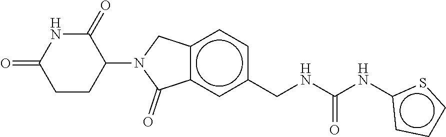 | 91 |
| 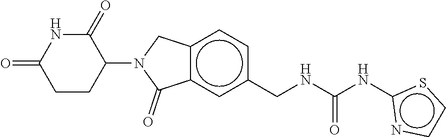 | 92 |
| 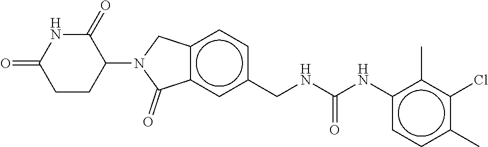 | 93 |
| 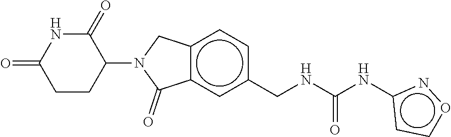 | 94 |
| 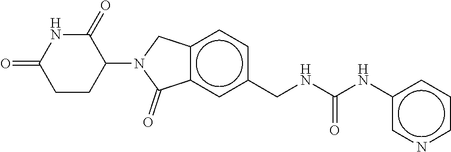 | 95 |
| 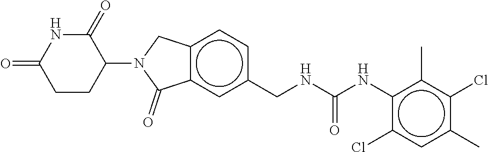 | 96 |
| 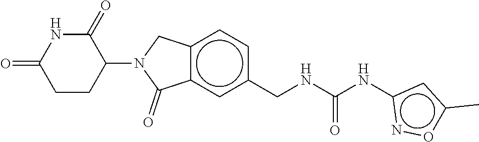 | 97 |
| 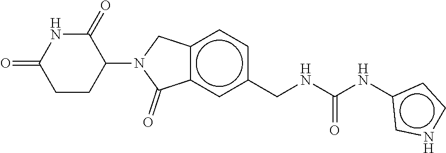 | 98 |
| 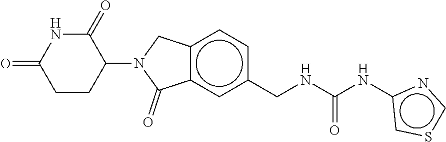 | 99 |
| 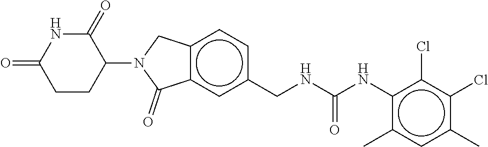 | 100 |
| 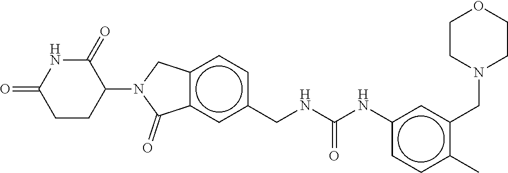 | 101 |
| 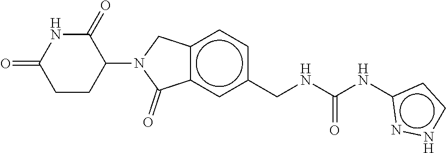 | 102 |
| 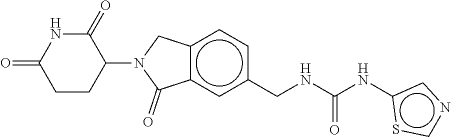 | 103 |
| 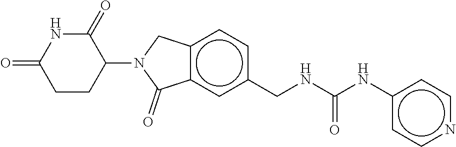 | 104 |
| 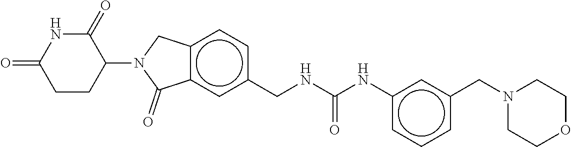 | 105 |
| 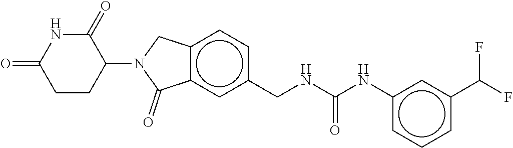 | 106 |
| 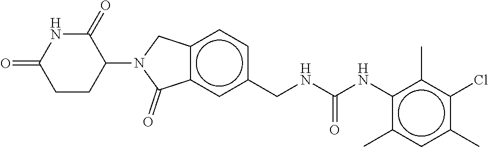 | 107 |
| 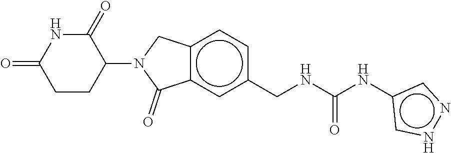 | 108 |
| 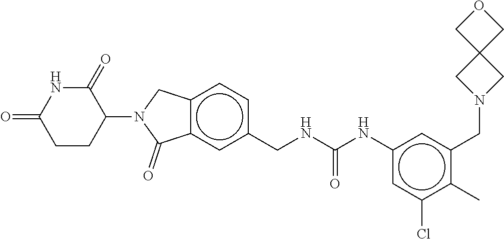 | 109 |
| 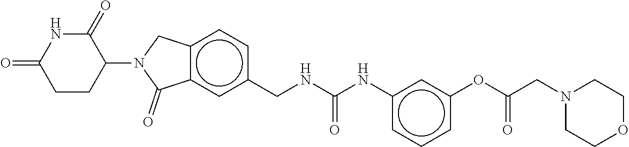 | 110 |
| 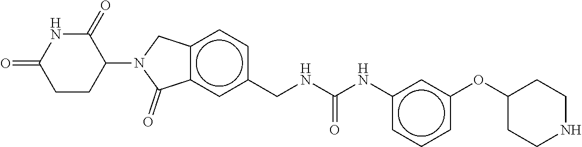 | 111 |
| 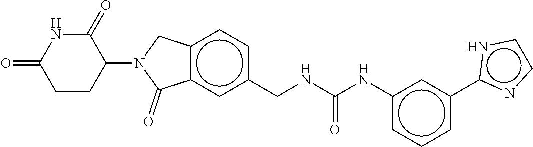 | 112 |
| 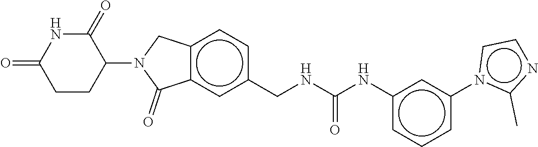 | 113 |
| 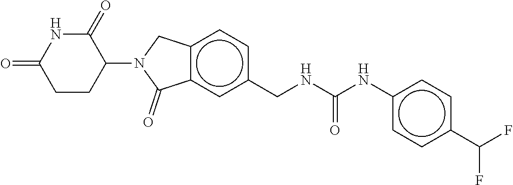 | 114 |
| 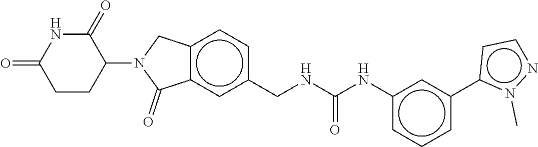 | 115 |
| 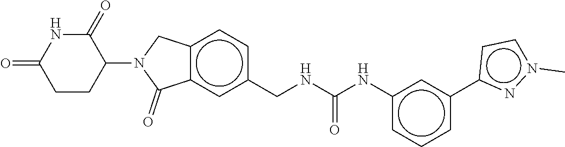 | 116 |
| 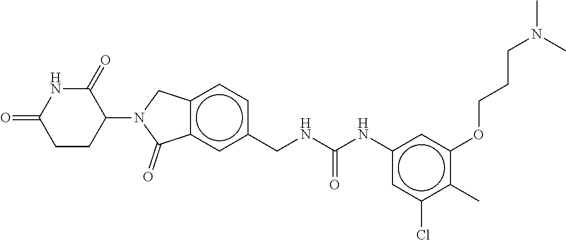 | 117 |
| 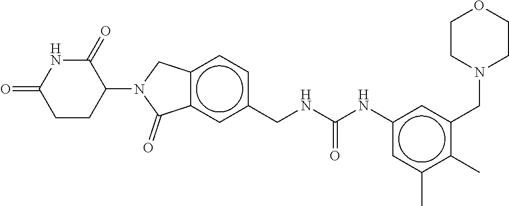 | 118 |
| 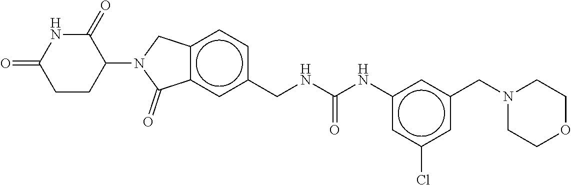 | 119 |
| 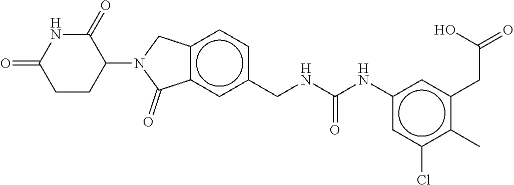 | 120 |
| 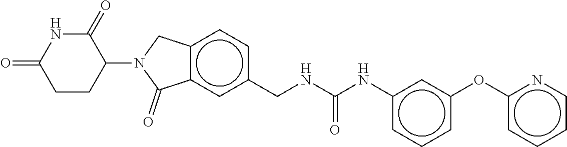 | 121 |
| 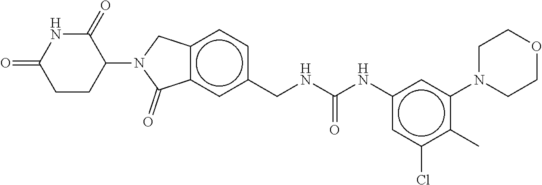 | 122 |
| 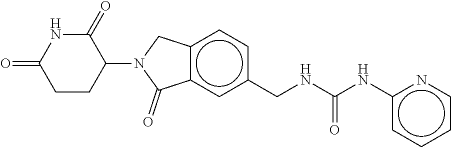 | 123 |
| 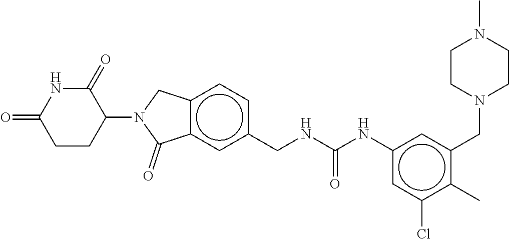 | 124 |
| 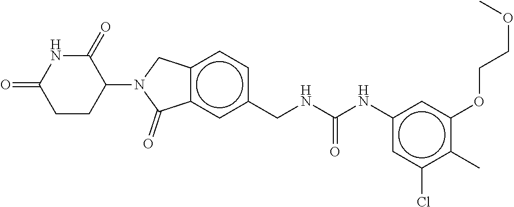 | 125 |
| 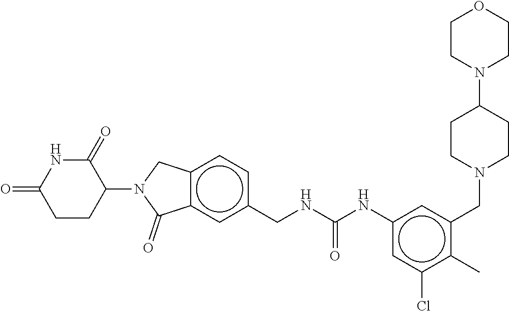 | 126 |
| 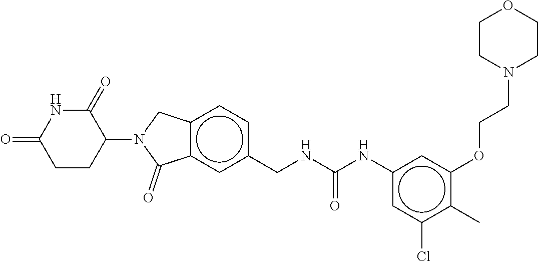 | 127 |
| 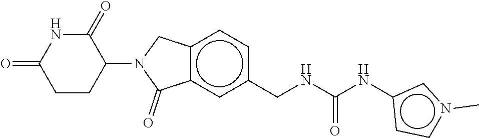 | 128 |
| 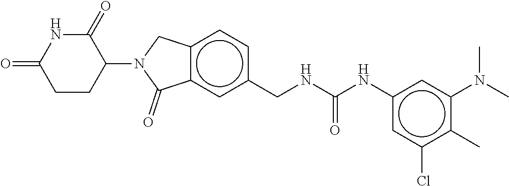 | 129 |
| 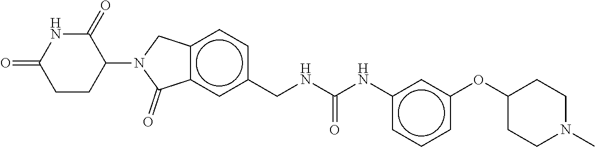 | 130 |
| 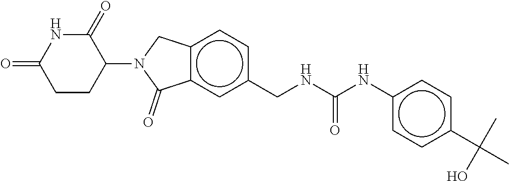 | 131 |
| 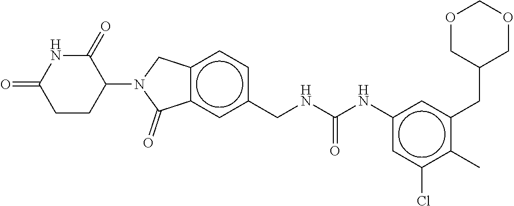 | 132 |
| 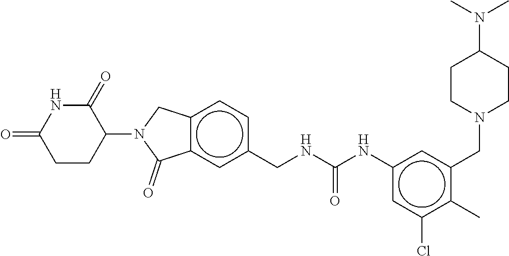 | 133 |
| 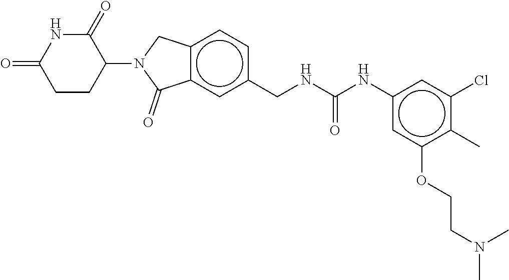 | 134 |
| 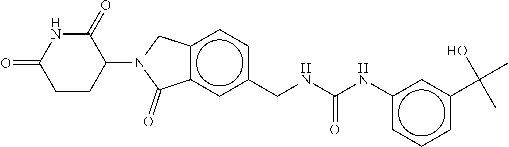 | 135 |
| 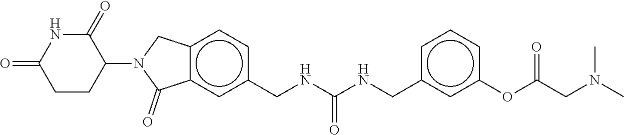 | 136 |
| 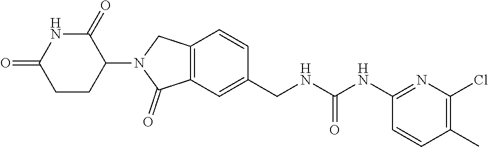 [SMILES: Cc1ccc(NC(=O)NCc2ccc3c(c2)C(=O)N(C2CCC(=O)NC2=O)C3)nc1Cl] | 137 |
| 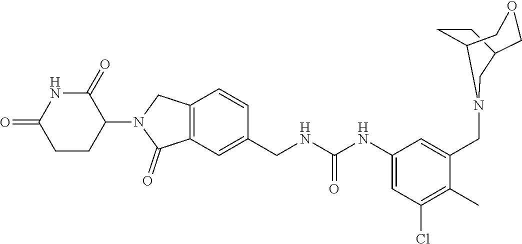 [SMILES: Cc1c(Cl)cc(NC(=O)NCc2ccc3c(c2)C(=O)N(C2CCC(=O)NC2=O)C3)cc1CN1CC2CCC1COC2] | 138 |
| 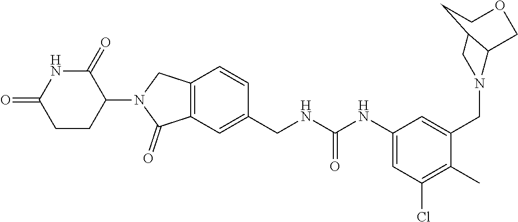 [SMILES: Cc1c(Cl)cc(NC(=O)NCc2ccc3c(c2)C(=O)N(C2CCC(=O)NC2=O)C3)cc1CN1CC2CCOCC21] | 139 |
| 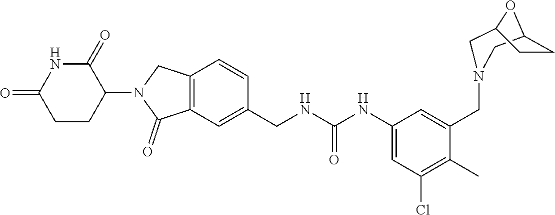 [SMILES: Cc1c(Cl)cc(NC(=O)NCc2ccc3c(c2)C(=O)N(C2CCC(=O)NC2=O)C3)cc1CN1CC2CCC(C1)O2] | 140 |
| 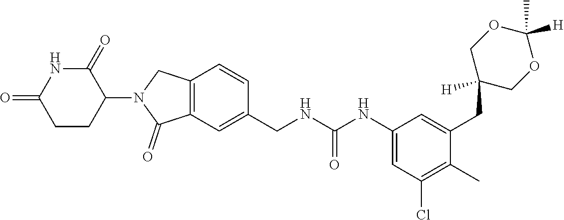 [SMILES: Cc1c(Cl)cc(NC(=O)NCc2ccc3c(c2)C(=O)N(C2CCC(=O)NC2=O)C3)cc1C[C@H]1CO[C@H](C)OC1] | 141 |
| 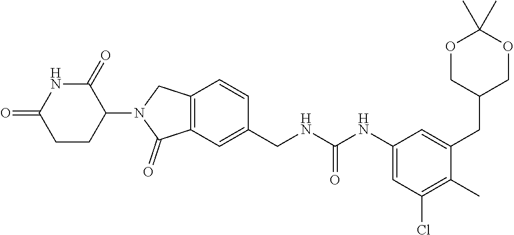 [SMILES: Cc1c(Cl)cc(NC(=O)NCc2ccc3c(c2)C(=O)N(C2CCC(=O)NC2=O)C3)cc1CC1COC(C)(C)OC1] | 142 |
| 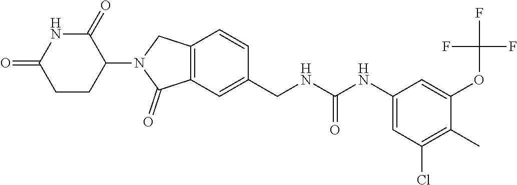 [SMILES: Cc1c(Cl)cc(NC(=O)NCc2ccc3c(c2)C(=O)N(C2CCC(=O)NC2=O)C3)cc1OC(F)(F)F] | 143 |
| 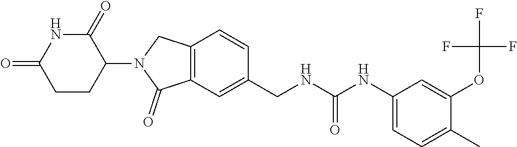 [SMILES: Cc1ccc(NC(=O)NCc2ccc3c(c2)C(=O)N(C2CCC(=O)NC2=O)C3)cc1OC(F)(F)F] | 144 |
| 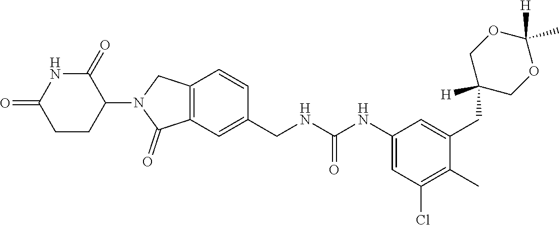 [SMILES: Cc1c(Cl)cc(NC(=O)NCc2ccc3c(c2)C(=O)N(C2CCC(=O)NC2=O)C3)cc1C[C@H]1CO[C@@H](C)OC1] | 145 |
| 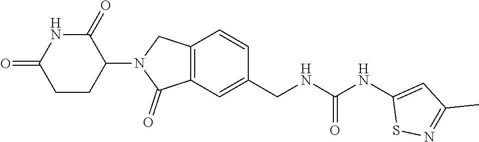 [SMILES: Cc1cc(NC(=O)NCc2ccc3c(c2)C(=O)N(C2CCC(=O)NC2=O)C3)sn1] | 146 |
| 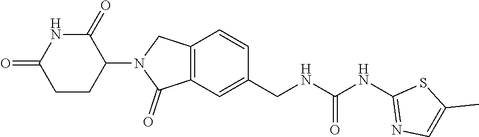 [SMILES: Cc1cnc(NC(=O)NCc2ccc3c(c2)C(=O)N(C2CCC(=O)NC2=O)C3)s1] | 147 |
| 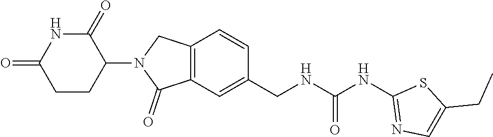 [SMILES: CCc1cnc(NC(=O)NCc2ccc3c(c2)C(=O)N(C2CCC(=O)NC2=O)C3)s1] | 148 |
| 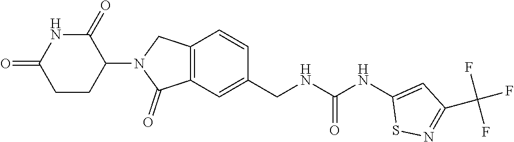 [SMILES: O=C1CCC(N2Cc3ccc(CNC(=O)Nc4cc(C(F)(F)F)ns4)cc3C2=O)C(=O)N1] | 149 |
| 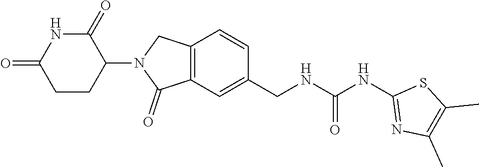 [SMILES: Cc1nc(NC(=O)NCc2ccc3c(c2)C(=O)N(C2CCC(=O)NC2=O)C3)sc1C] | 150 |
| 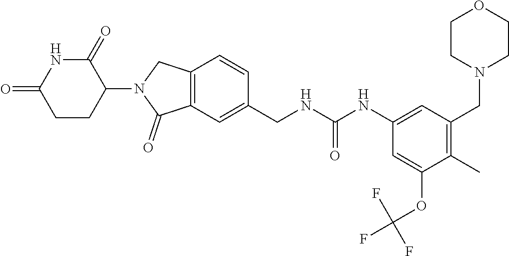 [SMILES: Cc1c(CN2CCOCC2)cc(NC(=O)NCc2ccc3c(c2)C(=O)N(C2CCC(=O)NC2=O)C3)cc1OC(F)(F)F] | 151 |
| 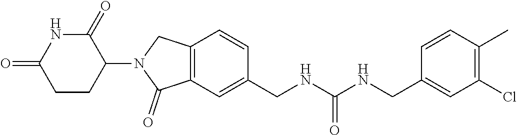 [SMILES: Cc1ccc(CNC(=O)NCc2ccc3c(c2)C(=O)N(C2CCC(=O)NC2=O)C3)cc1Cl] | 152 |
| 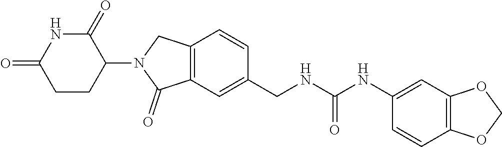 [SMILES: O=C1CCC(N2Cc3ccc(CNC(=O)Nc4ccc5c(c4)OCO5)cc3C2=O)C(=O)N1] | 153 |
| 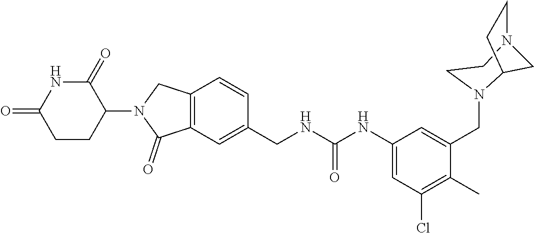 [SMILES: Cc1c(Cl)cc(NC(=O)NCc2ccc3c(c2)C(=O)N(C2CCC(=O)NC2=O)C3)cc1CN1CCN2CCC1C2] | 154 |
| 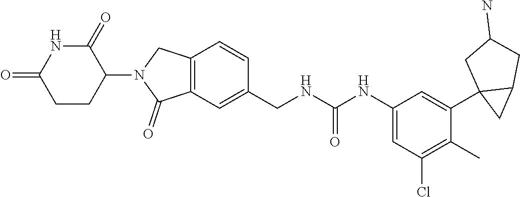 [SMILES: Cc1c(Cl)cc(NC(=O)NCc2ccc3c(c2)C(=O)N(C2CCC(=O)NC2=O)C3)cc1C12CC(N)CC1C2] | 155 |
| 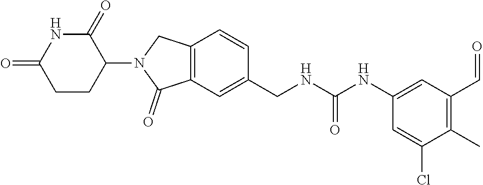 [SMILES: Cc1c(Cl)cc(NC(=O)NCc2ccc3c(c2)C(=O)N(C2CCC(=O)NC2=O)C3)cc1C=O] | 156 |
| 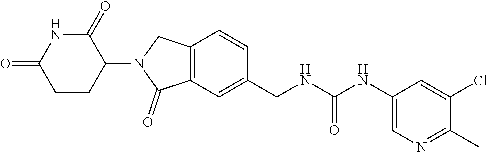 [SMILES: Cc1ncc(NC(=O)NCc2ccc3c(c2)C(=O)N(C2CCC(=O)NC2=O)C3)cc1Cl] | 157 |
| 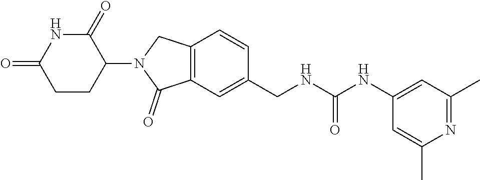 [SMILES: Cc1cc(NC(=O)NCc2ccc3c(c2)C(=O)N(C2CCC(=O)NC2=O)C3)cc(C)n1] | 158 |
| 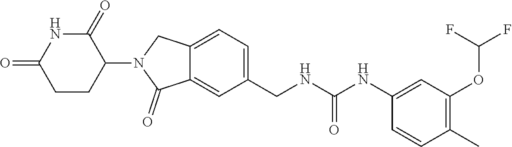 [SMILES: Cc1ccc(NC(=O)NCc2ccc3c(c2)C(=O)N(C2CCC(=O)NC2=O)C3)cc1OC(F)F] | 159 |
| 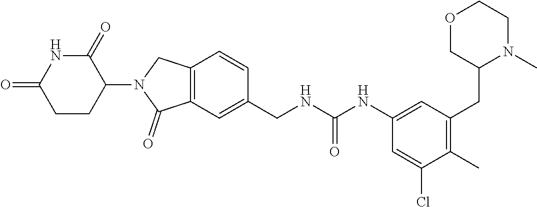 [SMILES: Cc1c(Cl)cc(NC(=O)NCc2ccc3c(c2)C(=O)N(C2CCC(=O)NC2=O)C3)cc1CC1COCCN1C] | 160 |

TABLE 2
Specific examples 200-307

| Compound | No. |
|---|---|
| 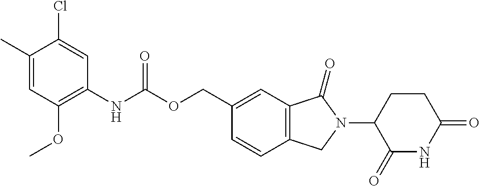 [SMILES: COc1cc(C)c(Cl)cc1NC(=O)OCc1ccc2c(c1)C(=O)N(C1CCC(=O)NC1=O)C2] | 201 |
| 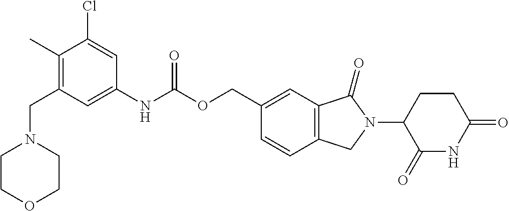 [SMILES: Cc1c(Cl)cc(NC(=O)OCc2ccc3c(c2)C(=O)N(C2CCC(=O)NC2=O)C3)cc1CN1CCOCC1] | 202 |
| 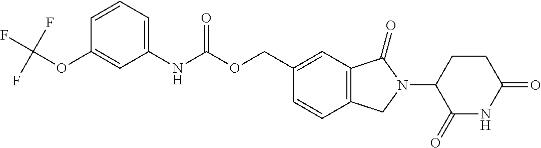 [SMILES: O=C1CCC(N2Cc3ccc(COC(=O)Nc4cccc(OC(F)(F)F)c4)cc3C2=O)C(=O)N1] | 203 |
| 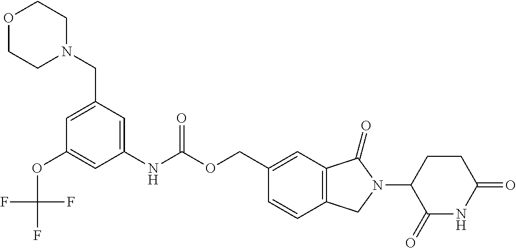 [SMILES: O=C1CCC(N2Cc3ccc(COC(=O)Nc4cc(CN5CCOCC5)cc(OC(F)(F)F)c4)cc3C2=O)C(=O)N1] | 204 |
| 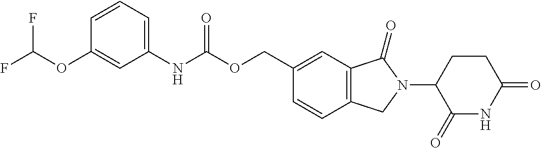 [SMILES: O=C1CCC(N2Cc3ccc(COC(=O)Nc4cccc(OC(F)F)c4)cc3C2=O)C(=O)N1] | 205 |
| 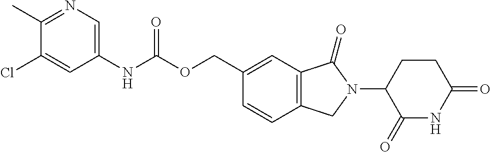 [SMILES: Cc1ncc(NC(=O)OCc2ccc3c(c2)C(=O)N(C2CCC(=O)NC2=O)C3)cc1Cl] | 206 |
| 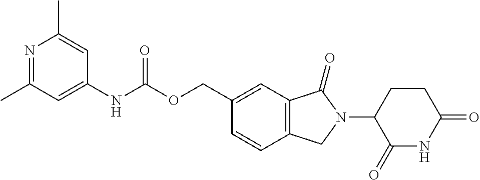 [SMILES: Cc1cc(NC(=O)OCc2ccc3c(c2)C(=O)N(C2CCC(=O)NC2=O)C3)cc(C)n1] | 207 |
| 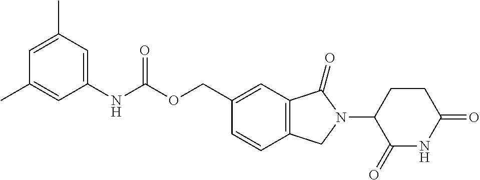 [SMILES: Cc1cc(C)cc(NC(=O)OCc2ccc3c(c2)C(=O)N(C2CCC(=O)NC2=O)C3)c1] | 208 |
| 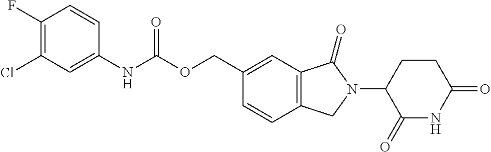 [SMILES: O=C1CCC(N2Cc3ccc(COC(=O)Nc4ccc(F)c(Cl)c4)cc3C2=O)C(=O)N1] | 209 |
| 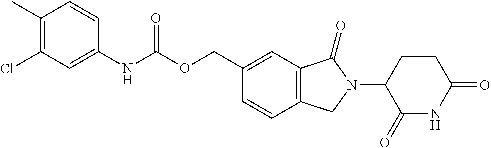 [SMILES: Cc1ccc(NC(=O)OCc2ccc3c(c2)C(=O)N(C2CCC(=O)NC2=O)C3)cc1Cl] | 210 |
| 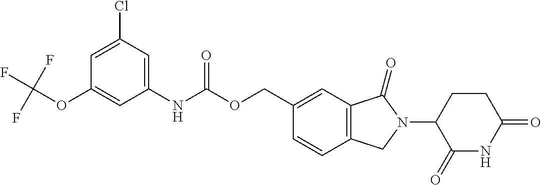 [SMILES: O=C1CCC(N2Cc3ccc(COC(=O)Nc4cc(Cl)cc(OC(F)(F)F)c4)cc3C2=O)C(=O)N1] | 211 |
| 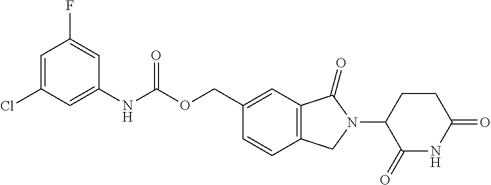 [SMILES: O=C1CCC(N2Cc3ccc(COC(=O)Nc4cc(F)cc(Cl)c4)cc3C2=O)C(=O)N1] | 212 |
| 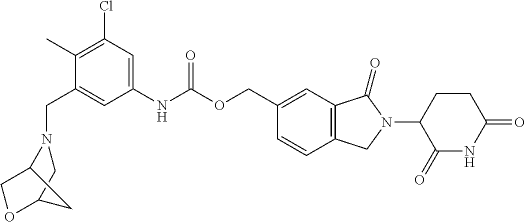 [SMILES: Cc1c(Cl)cc(NC(=O)OCc2ccc3c(c2)C(=O)N(C2CCC(=O)NC2=O)C3)cc1CN1CC2CC1CO2] | 213 |
| 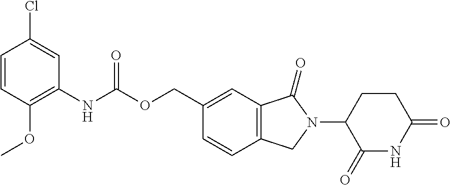 [SMILES: COc1ccc(Cl)cc1NC(=O)OCc1ccc2c(c1)C(=O)N(C1CCC(=O)NC1=O)C2] | 214 |
| 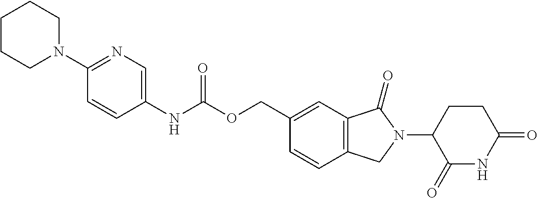 [SMILES: O=C1CCC(N2Cc3ccc(COC(=O)Nc4ccc(N5CCCCC5)nc4)cc3C2=O)C(=O)N1] | 215 |
| 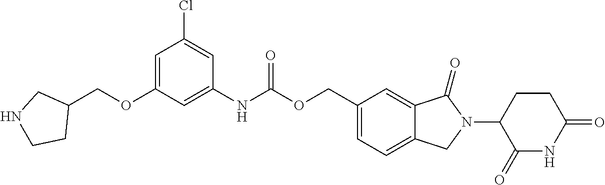 [SMILES: O=C1CCC(N2Cc3ccc(COC(=O)Nc4cc(Cl)cc(OCC5CCNC5)c4)cc3C2=O)C(=O)N1] | 216 |
| 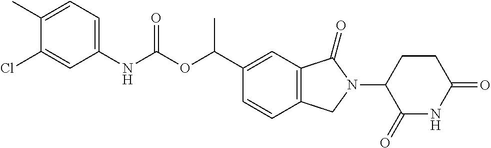 [SMILES: Cc1ccc(NC(=O)OC(C)c2ccc3c(c2)C(=O)N(C2CCC(=O)NC2=O)C3)cc1Cl] | 217 |
| 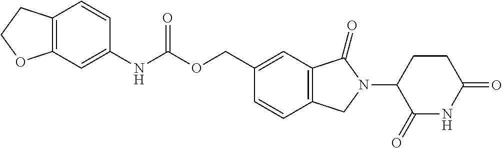 [SMILES: O=C1CCC(N2Cc3ccc(COC(=O)Nc4ccc5c(c4)OCC5)cc3C2=O)C(=O)N1] | 218 |
| 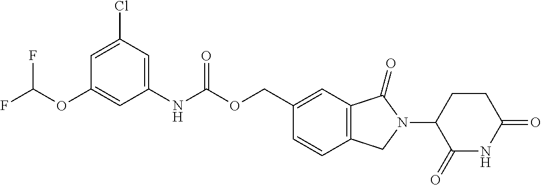 [SMILES: O=C1CCC(N2Cc3ccc(COC(=O)Nc4cc(Cl)cc(OC(F)F)c4)cc3C2=O)C(=O)N1] | 219 |
| 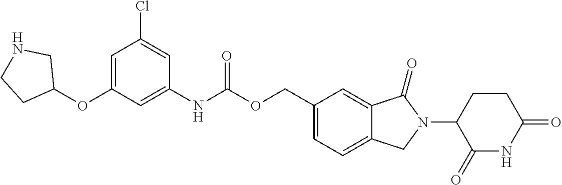 [SMILES: O=C1CCC(N2Cc3ccc(COC(=O)Nc4cc(Cl)cc(OC5CCNC5)c4)cc3C2=O)C(=O)N1] | 220 |
| 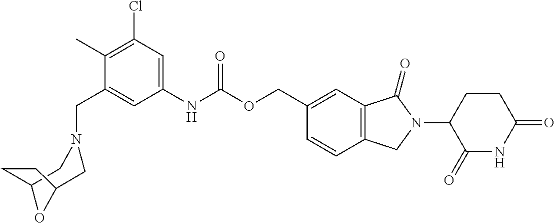 [SMILES: Cc1c(Cl)cc(NC(=O)OCc2ccc3c(c2)C(=O)N(C2CCC(=O)NC2=O)C3)cc1CN1CC2CCC(C1)O2] | 221 |
| 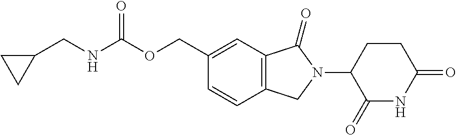 [SMILES: O=C1CCC(N2Cc3ccc(COC(=O)NCC4CC4)cc3C2=O)C(=O)N1] | 222 |
| 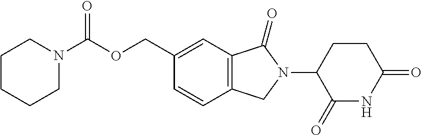 [SMILES: O=C1CCC(N2Cc3ccc(COC(=O)N4CCCCC4)cc3C2=O)C(=O)N1] | 223 |
| 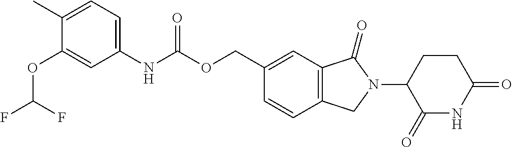 [SMILES: Cc1ccc(NC(=O)OCc2ccc3c(c2)C(=O)N(C2CCC(=O)NC2=O)C3)cc1OC(F)F] | 224 |
| 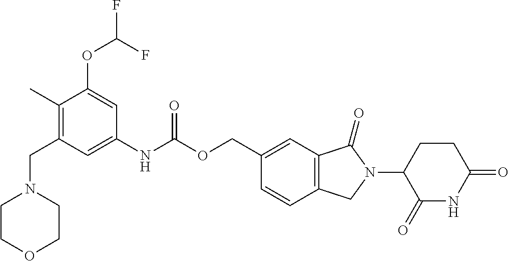 [SMILES: Cc1c(CN2CCOCC2)cc(NC(=O)OCc2ccc3c(c2)C(=O)N(C2CCC(=O)NC2=O)C3)cc1OC(F)F] | 225 |
| 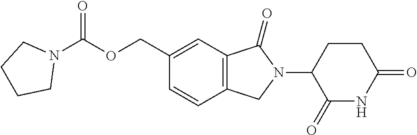 [SMILES: O=C1CCC(N2Cc3ccc(COC(=O)N4CCCC4)cc3C2=O)C(=O)N1] | 226 |
| 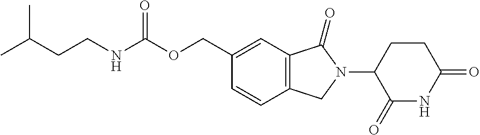 [SMILES: CC(C)CCNC(=O)OCc1ccc2c(c1)C(=O)N(C1CCC(=O)NC1=O)C2] | 227 |
| 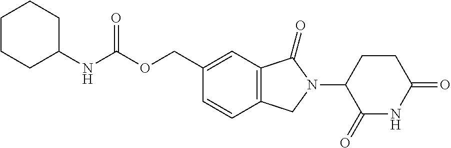 [SMILES: O=C1CCC(N2Cc3ccc(COC(=O)NC4CCCCC4)cc3C2=O)C(=O)N1] | 228 |
| 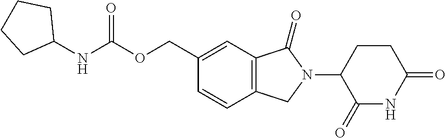 [SMILES: O=C1CCC(N2Cc3ccc(COC(=O)NC4CCCC4)cc3C2=O)C(=O)N1] | 229 |
| 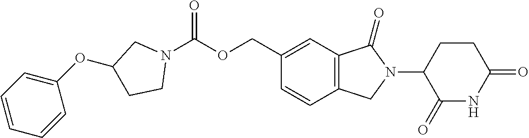 [SMILES: O=C1CCC(N2Cc3ccc(COC(=O)N4CCC(Oc5ccccc5)C4)cc3C2=O)C(=O)N1] | 230 |
| 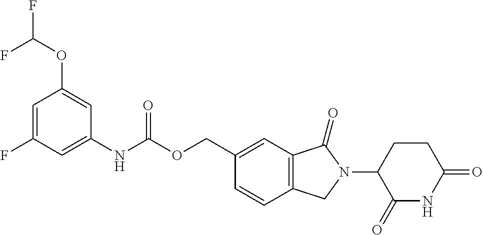 [SMILES: O=C1CCC(N2Cc3ccc(COC(=O)Nc4cc(F)cc(OC(F)F)c4)cc3C2=O)C(=O)N1] | 231 |
| 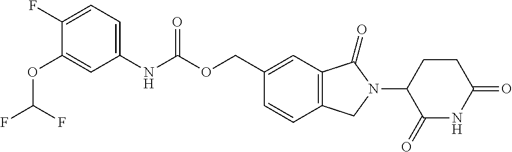 [SMILES: O=C1CCC(N2Cc3ccc(COC(=O)Nc4ccc(F)c(OC(F)F)c4)cc3C2=O)C(=O)N1] | 232 |
| 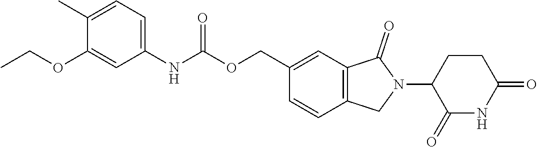 [SMILES: CCOc1cc(NC(=O)OCc2ccc3c(c2)C(=O)N(C2CCC(=O)NC2=O)C3)ccc1C] | 233 |
| 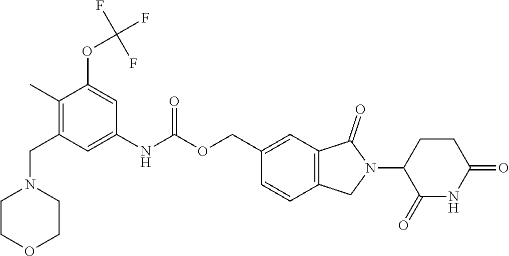 [SMILES: Cc1c(CN2CCOCC2)cc(NC(=O)OCc2ccc3c(c2)C(=O)N(C2CCC(=O)NC2=O)C3)cc1OC(F)(F)F] | 234 |
| 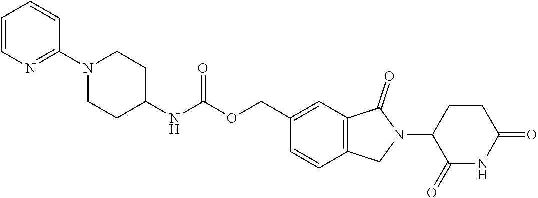 [SMILES: O=C1CCC(N2Cc3ccc(COC(=O)NC4CCN(c5ccccn5)CC4)cc3C2=O)C(=O)N1] | 235 |
| 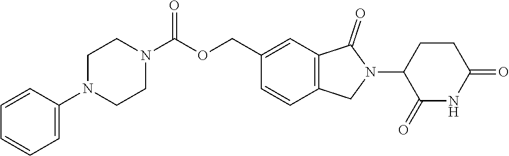 [SMILES: O=C1CCC(N2Cc3ccc(COC(=O)N4CCN(c5ccccc5)CC4)cc3C2=O)C(=O)N1] | 236 |
| 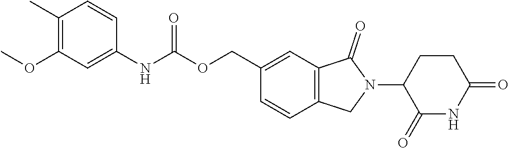 [SMILES: COc1cc(NC(=O)OCc2ccc3c(c2)C(=O)N(C2CCC(=O)NC2=O)C3)ccc1C] | 237 |
| 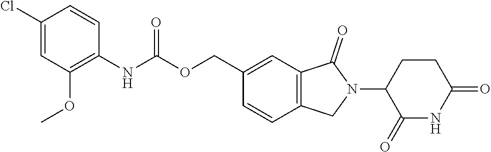 [SMILES: COc1cc(Cl)ccc1NC(=O)OCc1ccc2c(c1)C(=O)N(C1CCC(=O)NC1=O)C2] | 238 |
| 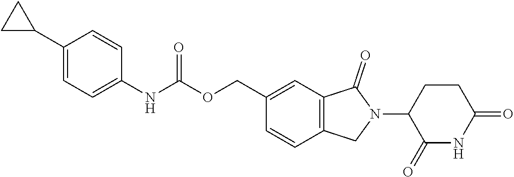 [SMILES: O=C1CCC(N2Cc3ccc(COC(=O)Nc4ccc(C5CC5)cc4)cc3C2=O)C(=O)N1] | 239 |
| 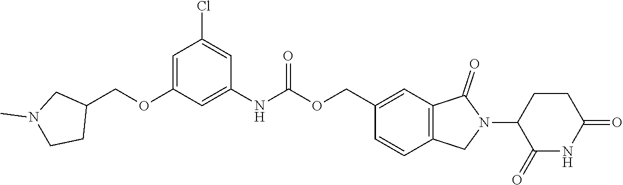 [SMILES: CN1CCC(COc2cc(Cl)cc(NC(=O)OCc3ccc4c(c3)C(=O)N(C3CCC(=O)NC3=O)C4)c2)C1] | 240 |
| 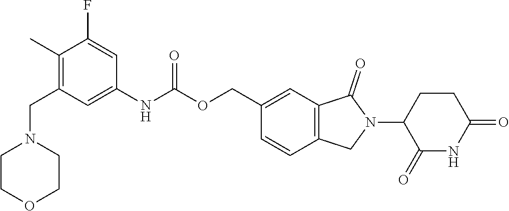 [SMILES: Cc1c(F)cc(NC(=O)OCc2ccc3c(c2)C(=O)N(C2CCC(=O)NC2=O)C3)cc1CN1CCOCC1] | 241 |
| 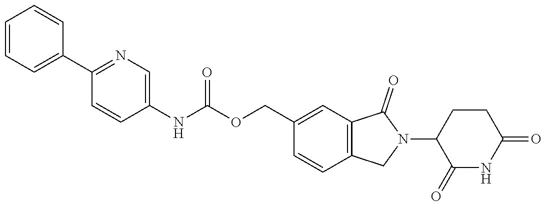 [SMILES: O=C1CCC(N2Cc3ccc(COC(=O)Nc4ccc(-c5ccccc5)nc4)cc3C2=O)C(=O)N1] | 242 |
| 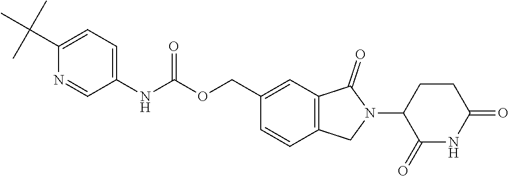 [SMILES: CC(C)(C)c1ccc(NC(=O)OCc2ccc3c(c2)C(=O)N(C2CCC(=O)NC2=O)C3)cn1] | 243 |
| 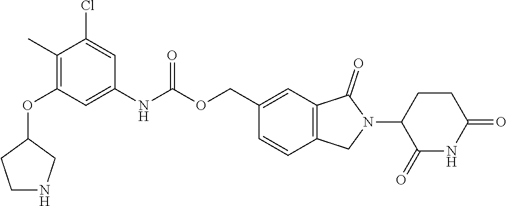 [SMILES: Cc1c(Cl)cc(NC(=O)OCc2ccc3c(c2)C(=O)N(C2CCC(=O)NC2=O)C3)cc1OC1CCNC1] | 244 |
| 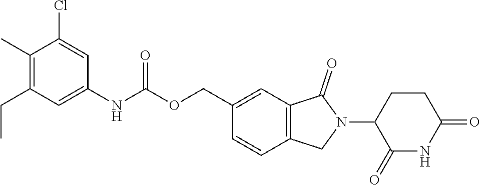 [SMILES: CCc1cc(NC(=O)OCc2ccc3c(c2)C(=O)N(C2CCC(=O)NC2=O)C3)cc(Cl)c1C] | 245 |
| 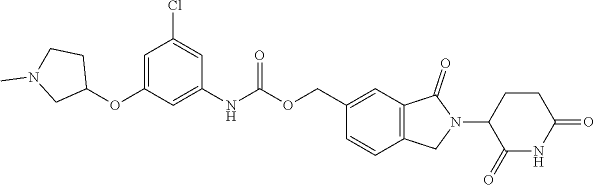 [SMILES: CN1CCC(Oc2cc(Cl)cc(NC(=O)OCc3ccc4c(c3)C(=O)N(C3CCC(=O)NC3=O)C4)c2)C1] | 246 |
| 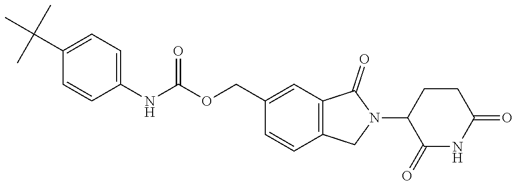 [SMILES: CC(C)(C)c1ccc(NC(=O)OCc2ccc3c(c2)C(=O)N(C2CCC(=O)NC2=O)C3)cc1] | 247 |
| 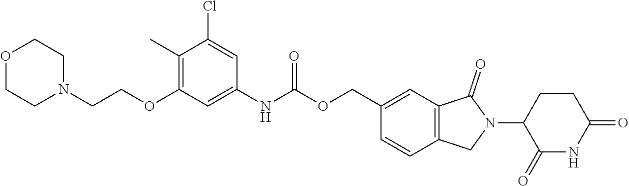 [SMILES: Cc1c(Cl)cc(NC(=O)OCc2ccc3c(c2)C(=O)N(C2CCC(=O)NC2=O)C3)cc1OCCN1CCOCC1] | 248 |
| 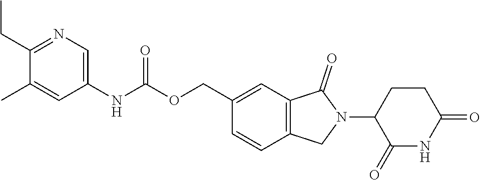 [SMILES: CCc1ncc(NC(=O)OCc2ccc3c(c2)C(=O)N(C2CCC(=O)NC2=O)C3)cc1C] | 249 |
| 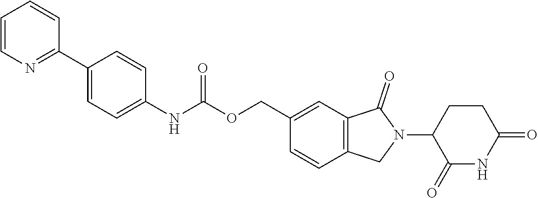 [SMILES: O=C1CCC(N2Cc3ccc(COC(=O)Nc4ccc(-c5ccccn5)cc4)cc3C2=O)C(=O)N1] | 250 |
| 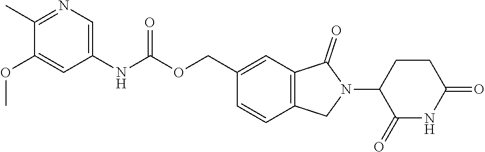 [SMILES: COc1cc(NC(=O)OCc2ccc3c(c2)C(=O)N(C2CCC(=O)NC2=O)C3)cnc1C] | 251 |
| 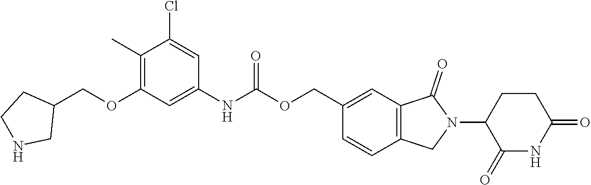 [SMILES: Cc1c(Cl)cc(NC(=O)OCc2ccc3c(c2)C(=O)N(C2CCC(=O)NC2=O)C3)cc1OCC1CCNC1] | 252 |
| 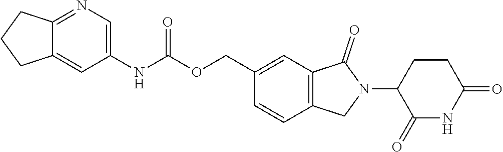 [SMILES: O=C1CCC(N2Cc3ccc(COC(=O)Nc4cnc5c(c4)CCC5)cc3C2=O)C(=O)N1] | 253 |
| 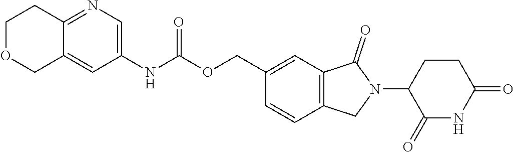 [SMILES: O=C1CCC(N2Cc3ccc(COC(=O)Nc4cnc5c(c4)COCC5)cc3C2=O)C(=O)N1] | 254 |
| 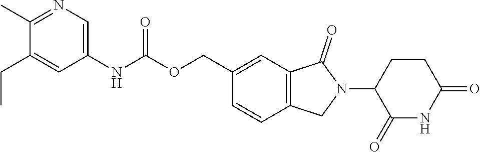 [SMILES: CCc1cc(NC(=O)OCc2ccc3c(c2)C(=O)N(C2CCC(=O)NC2=O)C3)cnc1C] | 255 |
| 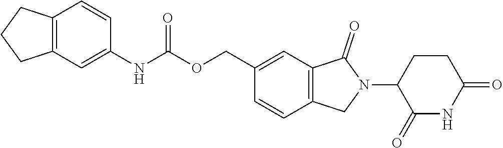 [SMILES: O=C1CCC(N2Cc3ccc(COC(=O)Nc4ccc5c(c4)CCC5)cc3C2=O)C(=O)N1] | 256 |
| 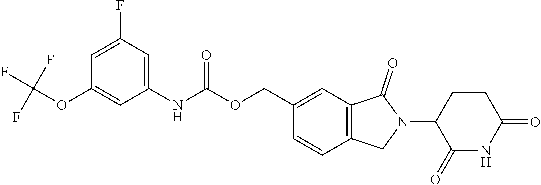 [SMILES: O=C1CCC(N2Cc3ccc(COC(=O)Nc4cc(F)cc(OC(F)(F)F)c4)cc3C2=O)C(=O)N1] | 257 |
| 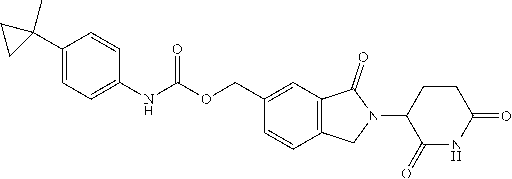 [SMILES: CC1(c2ccc(NC(=O)OCc3ccc4c(c3)C(=O)N(C3CCC(=O)NC3=O)C4)cc2)CC1] | 258 |
| 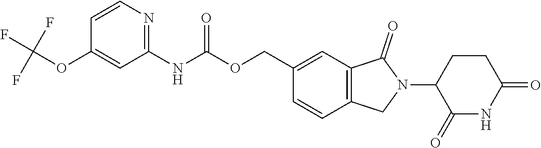 [SMILES: O=C1CCC(N2Cc3ccc(COC(=O)Nc4cc(OC(F)(F)F)ccn4)cc3C2=O)C(=O)N1] | 259 |
| 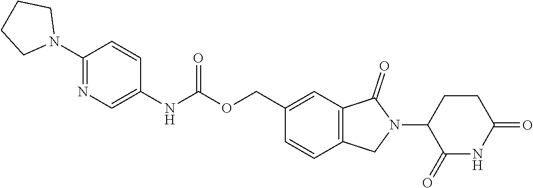 [SMILES: O=C1CCC(N2Cc3ccc(COC(=O)Nc4ccc(N5CCCC5)nc4)cc3C2=O)C(=O)N1] | 260 |
| 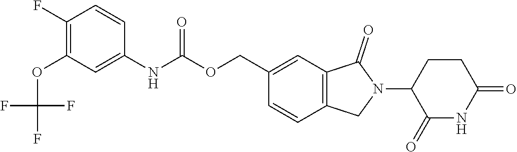 [SMILES: O=C1CCC(N2Cc3ccc(COC(=O)Nc4ccc(F)c(OC(F)(F)F)c4)cc3C2=O)C(=O)N1] | 261 |
| 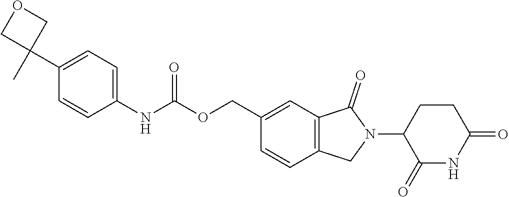 [SMILES: CC1(c2ccc(NC(=O)OCc3ccc4c(c3)C(=O)N(C3CCC(=O)NC3=O)C4)cc2)COC1] | 262 |
| 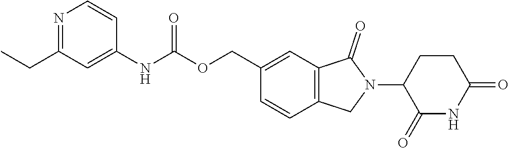 [SMILES: CCc1cc(NC(=O)OCc2ccc3c(c2)C(=O)N(C2CCC(=O)NC2=O)C3)ccn1] | 263 |
| 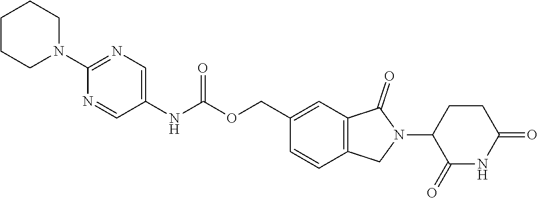 [SMILES: O=C1CCC(N2Cc3ccc(COC(=O)Nc4cnc(N5CCCCC5)nc4)cc3C2=O)C(=O)N1] | 264 |
| 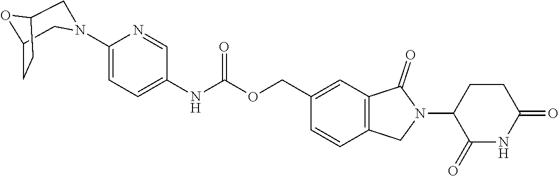 [SMILES: O=C1CCC(N2Cc3ccc(COC(=O)Nc4ccc(N5CC6CCC(C5)O6)nc4)cc3C2=O)C(=O)N1] | 265/ |
| 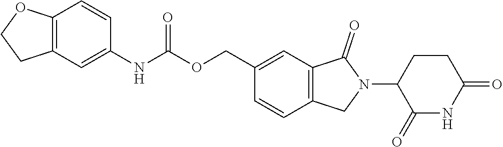 [SMILES: O=C1CCC(N2Cc3ccc(COC(=O)Nc4ccc5c(c4)CCO5)cc3C2=O)C(=O)N1] | 266 |
| 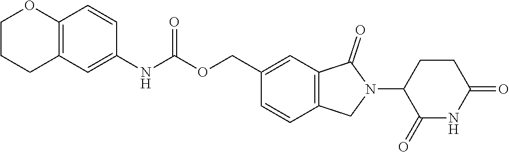 [SMILES: O=C1CCC(N2Cc3ccc(COC(=O)Nc4ccc5c(c4)CCCO5)cc3C2=O)C(=O)N1] | 267 |
| 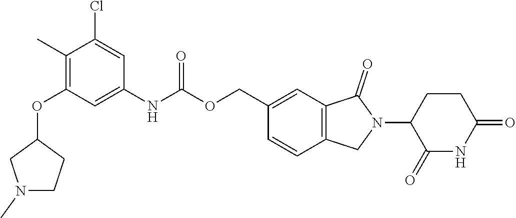 [SMILES: Cc1c(Cl)cc(NC(=O)OCc2ccc3c(c2)C(=O)N(C2CCC(=O)NC2=O)C3)cc1OC1CCN(C)C1] | 268 |
| 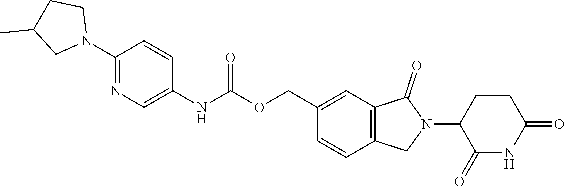 [SMILES: CC1CCN(c2ccc(NC(=O)OCc3ccc4c(c3)C(=O)N(C3CCC(=O)NC3=O)C4)cn2)C1] | 269 |
| 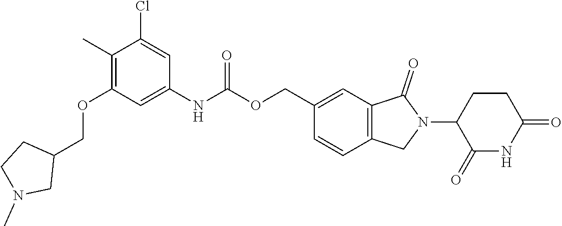 [SMILES: Cc1c(Cl)cc(NC(=O)OCc2ccc3c(c2)C(=O)N(C2CCC(=O)NC2=O)C3)cc1OCC1CCN(C)C1] | 270 |
| 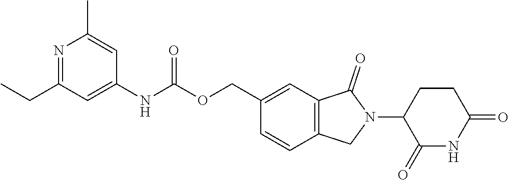 [SMILES: CCc1cc(NC(=O)OCc2ccc3c(c2)C(=O)N(C2CCC(=O)NC2=O)C3)cc(C)n1] | 271 |
| 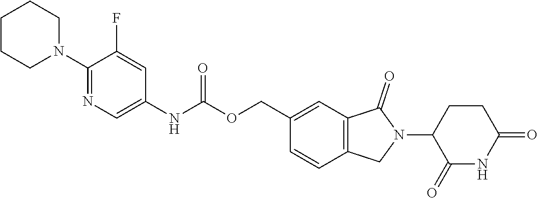 [SMILES: O=C1CCC(N2Cc3ccc(COC(=O)Nc4cnc(N5CCCCC5)c(F)c4)cc3C2=O)C(=O)N1] | 272 |
| 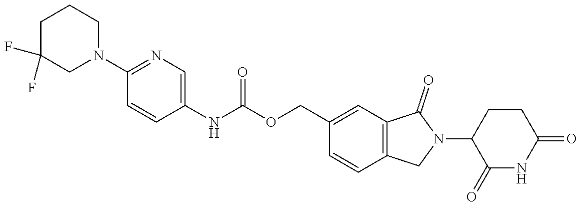 [SMILES: O=C1CCC(N2Cc3ccc(COC(=O)Nc4ccc(N5CCCC(F)(F)C5)nc4)cc3C2=O)C(=O)N1] | 273 |
| 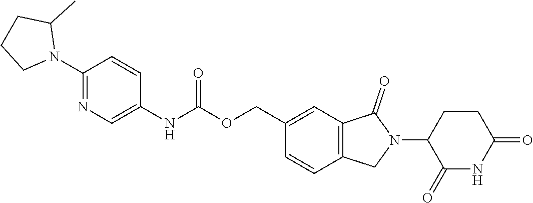 [SMILES: CC1CCCN1c1ccc(NC(=O)OCc2ccc3c(c2)C(=O)N(C2CCC(=O)NC2=O)C3)cn1] | 274 |
| 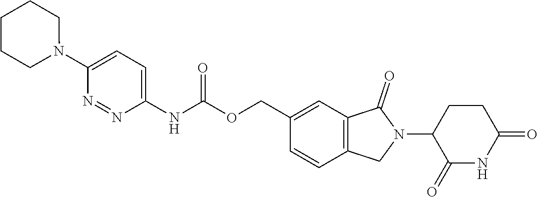 [SMILES: O=C1CCC(N2Cc3ccc(COC(=O)Nc4ccc(N5CCCCC5)nn4)cc3C2=O)C(=O)N1] | 275 |
| 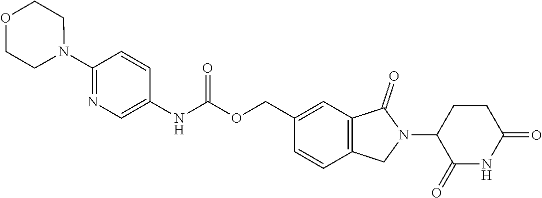 [SMILES: O=C1CCC(N2Cc3ccc(COC(=O)Nc4ccc(N5CCOCC5)nc4)cc3C2=O)C(=O)N1] | 276 |
| 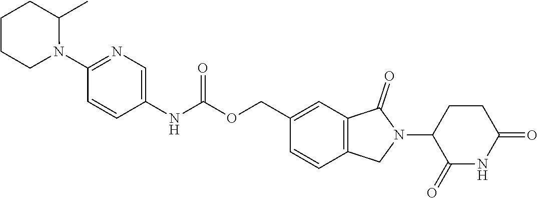 [SMILES: CC1CCCCN1c1ccc(NC(=O)OCc2ccc3c(c2)C(=O)N(C2CCC(=O)NC2=O)C3)cn1] | 277 |
| 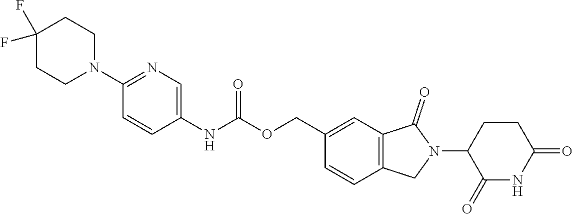 [SMILES: O=C1CCC(N2Cc3ccc(COC(=O)Nc4ccc(N5CCC(F)(F)CC5)nc4)cc3C2=O)C(=O)N1] | 278 |
| 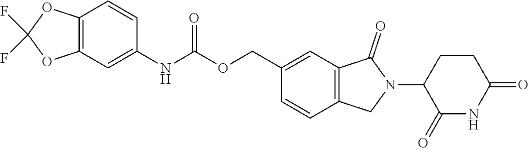 [SMILES: O=C1CCC(N2Cc3ccc(COC(=O)Nc4ccc5c(c4)OC(F)(F)O5)cc3C2=O)C(=O)N1] | 279 |
| 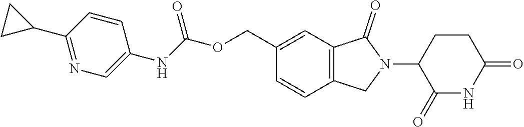 [SMILES: O=C1CCC(N2Cc3ccc(COC(=O)Nc4ccc(C5CC5)nc4)cc3C2=O)C(=O)N1] | 280 |
| 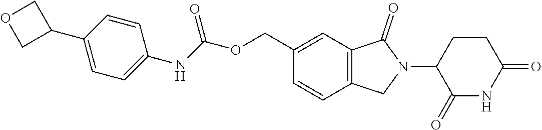 [SMILES: O=C1CCC(N2Cc3ccc(COC(=O)Nc4ccc(C5COC5)cc4)cc3C2=O)C(=O)N1] | 281 |
| 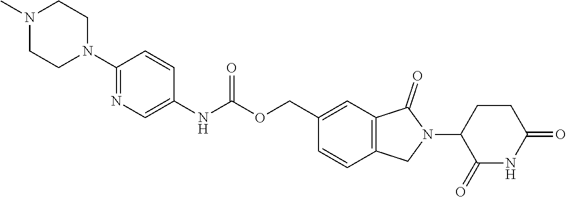 [SMILES: CN1CCN(c2ccc(NC(=O)OCc3ccc4c(c3)C(=O)N(C3CCC(=O)NC3=O)C4)cn2)CC1] | 282 |
| 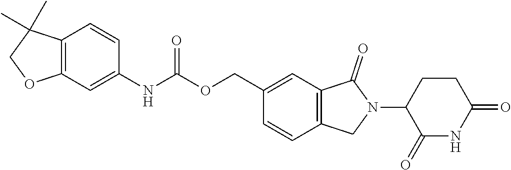 [SMILES: CC1(C)COc2cc(NC(=O)OCc3ccc4c(c3)C(=O)N(C3CCC(=O)NC3=O)C4)ccc21] | 283 |
| 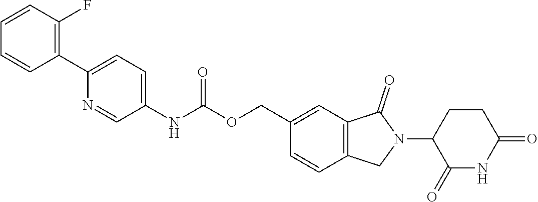 [SMILES: O=C1CCC(N2Cc3ccc(COC(=O)Nc4ccc(-c5ccccc5F)nc4)cc3C2=O)C(=O)N1] | 284 |
| 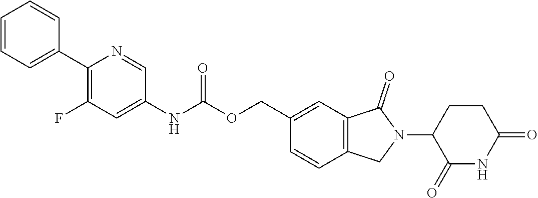 [SMILES: O=C1CCC(N2Cc3ccc(COC(=O)Nc4cnc(-c5ccccc5)c(F)c4)cc3C2=O)C(=O)N1] | 285 |
| 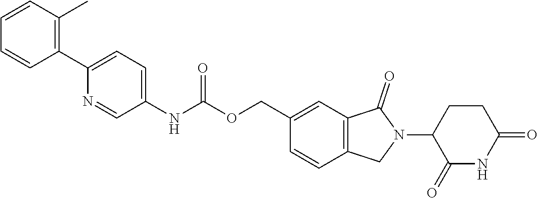 [SMILES: Cc1ccccc1-c1ccc(NC(=O)OCc2ccc3c(c2)C(=O)N(C2CCC(=O)NC2=O)C3)cn1] | 286 |
| 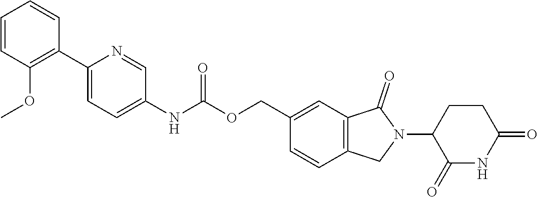 [SMILES: COc1ccccc1-c1ccc(NC(=O)OCc2ccc3c(c2)C(=O)N(C2CCC(=O)NC2=O)C3)cn1] | 287 |
| 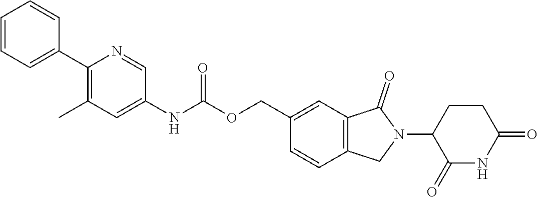 [SMILES: Cc1cc(NC(=O)OCc2ccc3c(c2)C(=O)N(C2CCC(=O)NC2=O)C3)cnc1-c1ccccc1] | 288 |
| 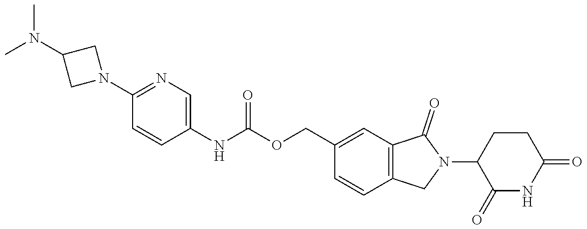 [SMILES: CN(C)C1CN(c2ccc(NC(=O)OCc3ccc4c(c3)C(=O)N(C3CCC(=O)NC3=O)C4)cn2)C1] | 289 |
| 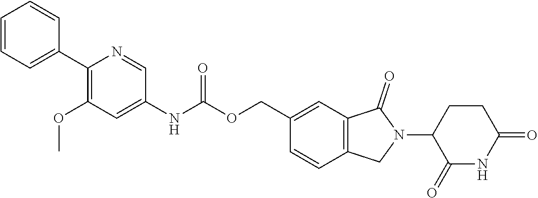 [SMILES: COc1cc(NC(=O)OCc2ccc3c(c2)C(=O)N(C2CCC(=O)NC2=O)C3)cnc1-c1ccccc1] | 290 |
| 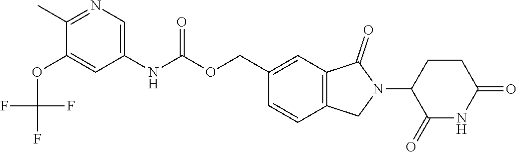 [SMILES: Cc1ncc(NC(=O)OCc2ccc3c(c2)C(=O)N(C2CCC(=O)NC2=O)C3)cc1OC(F)(F)F] | 291 |
| 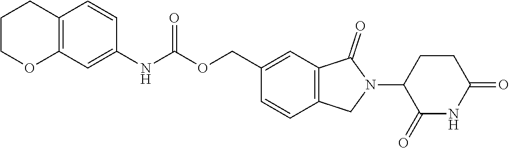 [SMILES: O=C1CCC(N2Cc3ccc(COC(=O)Nc4ccc5c(c4)OCCC5)cc3C2=O)C(=O)N1] | 292 |
| 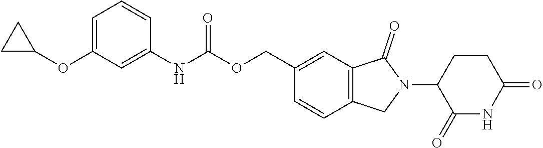 [SMILES: O=C1CCC(N2Cc3ccc(COC(=O)Nc4cccc(OC5CC5)c4)cc3C2=O)C(=O)N1] | 293 |
| 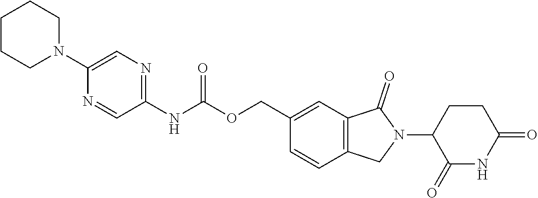 [SMILES: O=C1CCC(N2Cc3ccc(COC(=O)Nc4cnc(N5CCCCC5)cn4)cc3C2=O)C(=O)N1] | 294 |
| 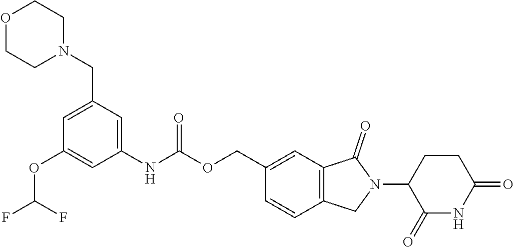 [SMILES: O=C1CCC(N2Cc3ccc(COC(=O)Nc4cc(CN5CCOCC5)cc(OC(F)F)c4)cc3C2=O)C(=O)N1] | 295 |
| 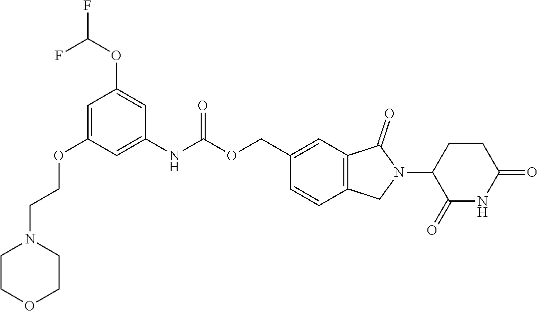 [SMILES: O=C1CCC(N2Cc3ccc(COC(=O)Nc4cc(OCCN5CCOCC5)cc(OC(F)F)c4)cc3C2=O)C(=O)N1] | 296 |
| 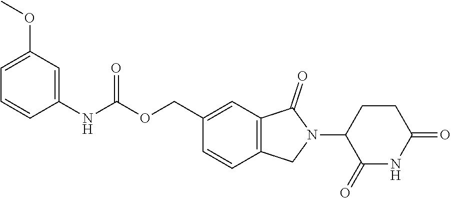 [SMILES: COc1cccc(NC(=O)OCc2ccc3c(c2)C(=O)N(C2CCC(=O)NC2=O)C3)c1] | 297 |
| 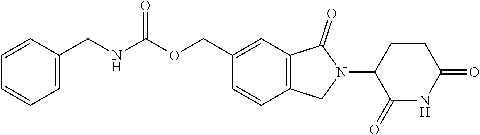 [SMILES: O=C1CCC(N2Cc3ccc(COC(=O)NCc4ccccc4)cc3C2=O)C(=O)N1] | 298 |
| 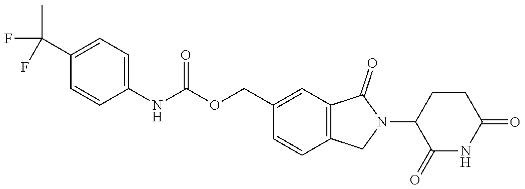 [SMILES: CC(F)(F)c1ccc(NC(=O)OCc2ccc3c(c2)C(=O)N(C2CCC(=O)NC2=O)C3)cc1] | 299 |
| 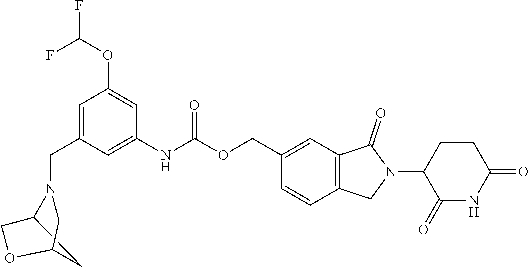 [SMILES: O=C1CCC(N2Cc3ccc(COC(=O)Nc4cc(CN5CC6CC5CO6)cc(OC(F)F)c4)cc3C2=O)C(=O)N1] | 300 |
| 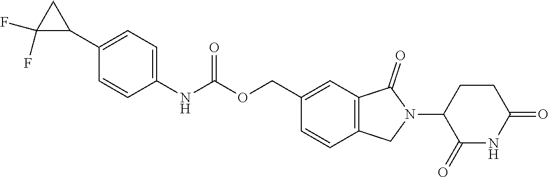 [SMILES: O=C1CCC(N2Cc3ccc(COC(=O)Nc4ccc(C5CC5(F)F)cc4)cc3C2=O)C(=O)N1] | 301 |
| 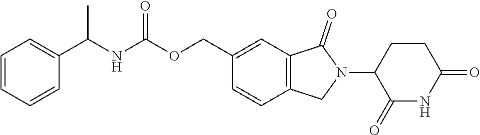 [SMILES: CC(NC(=O)OCc1ccc2c(c1)C(=O)N(C1CCC(=O)NC1=O)C2)c1ccccc1] | 302 |
| 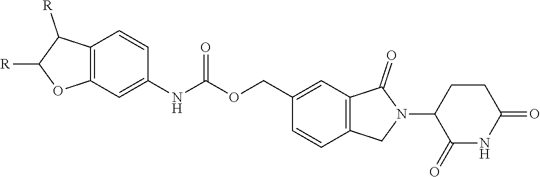 | 303 |
| R = CH₃ or H | |
| 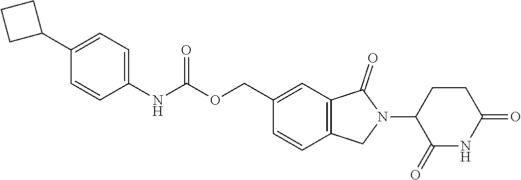 [SMILES: O=C1CCC(N2Cc3ccc(COC(=O)Nc4ccc(C5CCC5)cc4)cc3C2=O)C(=O)N1] | 304 |
| 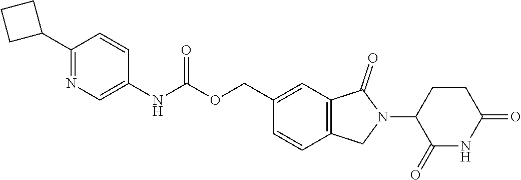 [SMILES: O=C1CCC(N2Cc3ccc(COC(=O)Nc4ccc(C5CCC5)nc4)cc3C2=O)C(=O)N1] | 305 |
| 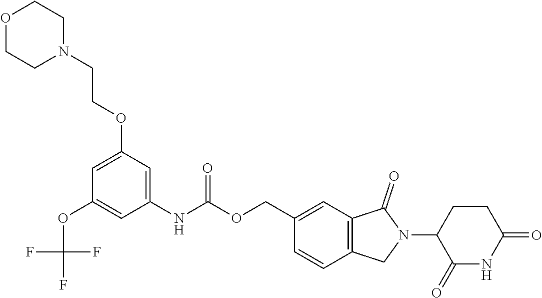 [SMILES: O=C1CCC(N2Cc3ccc(COC(=O)Nc4cc(OCCN5CCOCC5)cc(OC(F)(F)F)c4)cc3C2=O)C(=O)N1] | 306 |
| 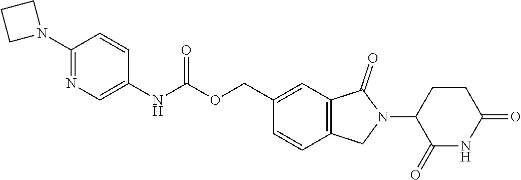 [SMILES: O=C1CCC(N2Cc3ccc(COC(=O)Nc4ccc(N5CCC5)nc4)cc3C2=O)C(=O)N1] | 307 |

Compound 1: General procedure A with variant i) was used for the preparation from compound VI-A employing phenyl-isocyanate. ¹H NMR (600 MHz, DMSO-d₆) δ=10.98 (s, 1H), 8.61 (s, 1H), 7.67 (s, 1H), 7.60-7.53 (m, 2H), 7.43-7.37 (m, 2H), 7.25-7.18 (m, 2H), 6.93-6.86 (m, 1H), 6.74 (t, J=6.1 Hz, 1H), 5.11 (dd, J=13.3, 5.1 Hz, 1H), 4.43 (d, J=17.1 Hz, 1H), 4.40 (d, J=6.0 Hz, 2H), 4.31 (d, J=17.1 Hz, 1H), 2.96-2.86 (m, 1H), 2.64-2.56 (m, 1H), 2.45-2.33 (m, 1H), 2.04-1.96 (m, 1H). MS (ESI) m/z 393.1 [M+H]⁺

Compound 2: General procedure A with variant i) was used for the preparation from compound VI-A employing 4-Chloro-phenyl-isocyanate. ¹H NMR (600 MHz, DMSO-d₆) δ 10.98 (s, 1H), 8.79 (s, 1H), 7.66 (s, 1H), 7.58-7.54 (m, 2H), 7.47-7.41 (m, 2H), 7.29-7.22 (m, 2H), 6.81 (t, J=6.0 Hz, 1H), 5.11 (dd, J=13.3, 5.1 Hz, 1H), 4.43 (d, J=17.1 Hz, 1H), 4.40 (d, J=6.0 Hz, 2H), 4.30 (d, J=17.1 Hz, 1H), 2.96-2.85 (m, 1H), 2.64-2.55 (m, 1H), 2.45-2.33 (m, 1H), 2.04-1.95 (m, 1H). MS (ESI) m/z 427.1 [M+H, Cl³⁵]⁺

Compound 3: General procedure A with variant i) was used for the preparation from compound VI-A employing 3-Chloro-phenyl-isocyanate. ¹H NMR (600 MHz, DMSO-d₆) δ=10.98 (s, 1H), 8.85 (s, 1H), 7.68-7.67 (m, 1H), 7.67-7.66 (m, 1H), 7.58-7.54 (m, 2H), 7.26-7.19 (m, 2H), 6.95-6.93 (m, 1H), 6.84 (t, J=6.1 Hz, 1H), 5.11 (dd, J=13.3, 5.1 Hz, 1H), 4.43 (d, J=17.1 Hz, 1H), 4.40 (d, J=6.0 Hz, 2H), 4.31 (d, J=17.1 Hz, 1H), 2.96-2.86 (m, 1H), 2.64-2.56 (m, 1H), 2.45-2.34 (m, 1H), 2.04-1.96 (m, 1H). MS (ESI) m/z 427.1 [M+H, Cl³⁵]⁺

Compound 4: General procedure A with variant i) was used for the preparation from compound VI-A employing 2-Chloro-phenyl-isocyanate. ¹H NMR (600 MHz, DMSO-d₆) δ=10.99 (s, 1H), 8.19-8.13 (m, 2H), 7.69 (d, J=1.2 Hz, 1H), 7.61-7.54 (m, 3H), 7.44-7.38 (m, 1H), 7.28-7.21 (m, 1H), 7.00-6.93 (m, 1H), 5.13 (dd, J=13.3, 5.1 Hz, 1H), 4.48-4.42 (m, 3H), 4.32 (d, J=17.1 Hz, 1H), 2.97-2.87 (m, 1H), 2.65-2.57 (m, 1H), 2.46-2.35 (m, 1H), 2.05-1.97 (m, 1H). MS (ESI) m/z 427.1 [M+H, Cl³⁵]⁺

Compound 5: General procedure A with variant i) was used for the preparation from compound VI-A employing 4-Methoxy-phenyl-isocyanate. ¹H NMR (600 MHz, DMSO-d₆) δ=10.98 (s, 1H), 8.41 (s, 1H), 7.68-7.64 (m, 1H), 7.58-7.54 (m, 2H), 7.35-7.27 (m, 2H), 6.86-6.78 (m, 2H), 6.64 (t, J=6.1 Hz, 1H), 5.11 (dd, J=13.3, 5.1 Hz, 1H), 4.43 (d, J=17.1 Hz, 1H), 4.38 (d, J=6.0 Hz, 2H), 4.30 (d, J=17.1 Hz, 1H), 3.69 (s, 3H), 2.96-2.86 (m, 1H), 2.64-2.56 (m, 1H), 2.45-2.34 (m, 1H), 2.04-1.96 (m, 1H). MS (ESI) m/z 423.1 [M+H]⁺

Compound 6: General procedure A with variant i) was used for the preparation from compound VI-A employing 3-Methoxy-phenyl-isocyanate. ¹H NMR (600 MHz, DMSO-d₆) δ=10.98 (s, 1H), 8.64 (s, 1H), 7.68-7.64 (m, 1H), 7.58-7.54 (m, 2H), 7.18-7.08 (m, 2H), 6.92-6.86 (m, 1H), 6.74 (t, J=6.0 Hz, 1H), 6.51-6.45 (m, 1H), 5.11 (dd, J=13.3, 5.1 Hz, 1H), 4.43 (d, J=17.1 Hz, 1H), 4.40 (d, J=6.0 Hz, 2H), 4.31 (d, J=17.1 Hz, 1H), 3.70 (s, 3H), 2.96-2.86 (m, 1H), 2.64-2.56 (m, 1H), 2.45-2.33 (m, 1H), 2.04-1.96 (m, 1H). MS (ESI) m/z 423.1 [M+H]⁺

Compound 7: General procedure A with variant i) was used for the preparation from compound VI-A employing 2-Methoxy-phenyl-isocyanate. ¹H NMR (600 MHz, DMSO-d₆) δ=10.98 (s, 1H), 8.11-8.07 (m, 1H), 8.05 (s, 1H), 7.67-7.64 (m, 1H), 7.58-7.54 (m, 2H), 7.41 (t, J=5.9 Hz, 1H), 6.98-6.95 (m, 1H), 6.90-6.86 (m, 1H), 6.85-6.81 (m, 1H), 5.12 (dd, J=13.3, 5.1 Hz, 1H), 4.44 (d, J=17.1 Hz, 1H), 4.41 (d, J=5.9 Hz, 2H), 4.31 (d, J=17.1 Hz, 1H), 3.84 (s, 3H), 2.95-2.85 (m, 1H), 2.64-2.56 (m, 1H), 2.44-2.35 (m, 1H), 2.03-1.97 (m, 1H). MS (ESI) m/z 423.1 [M+H]⁺

Compound 8: General procedure A with variant ii) was used for the preparation from compound VI-A employing 4-Chloro-3-methyl-phenyl-isocyanate. ¹H NMR (400 MHz, DMSO-d₆) δ=10.99 (s, 1H), 8.75 (s, 1H), 7.67 (s, 2H), 7.57 (s, 2H), 7.22-7.11 (m, 2H), 6.82 (br t, J=5.9 Hz, 1H), 5.12 (dd, J=5.1, 13.3 Hz, 1H), 4.48-4.36 (m, 3H), 4.36-4.26 (m, 1H), 2.98-2.85 (m, 1H), 2.60 (br dd, J=2.0, 15.5 Hz, 1H), 2.46-2.37 (m, 1H), 2.24 (s, 3H), 2.06-1.95 (m, 1H). MS (ESI) m/z 441.2 [M+H]⁺

Compound 9: General procedure A with variant ii) was used for the preparation from compound VI-A employing 4-Bromo-3-methyl-phenyl-isocyanate. ¹H NMR (400 MHz, DMSO-d₆) δ=10.98 (s, 1H), 8.52 (s, 1H), 7.66 (s, 1H), 7.56 (d, J=0.9 Hz, 2H), 7.34-7.27 (m, 2H), 7.26-7.20 (m, 2H), 6.68 (t, J=6.0 Hz, 1H), 5.11 (dd, J=5.1, 13.3 Hz, 1H), 4.48-4.34 (m, 3H), 4.34-4.26 (m, 1H), 2.96-2.84 (m, 1H), 2.59 (td, J=1.9, 15.4 Hz, 1H), 2.43-2.36 (m, 1H), 2.04-1.94 (m, 1H), 2.04-1.94 (m, 1H), 1.24 (s, 9H). MS (ESI) m/z 449.3 [M+H]⁺

Compound 10: General procedure A with variant ii) was used for the preparation from compound VI-A employing tert-Butyl-isocyanate. ¹H NMR (400 MHz, DMSO-d₆) δ=10.98 (s, 1H), 7.59 (s, 1H), 7.56-7.52 (m, 1H), 7.51-7.47 (m, 1H), 6.20 (t, J=6.1 Hz, 1H), 5.77 (s, 1H), 5.10 (dd, J=5.0, 13.2 Hz, 1H), 4.46-4.30 (m, 2H), 4.27 (d, J=6.1 Hz, 2H), 2.96-2.86 (m, 1H), 2.62 (br s, 1H), 2.43-2.34 (m, 1H), 2.04-1.96 (m, 1H), 1.23 (s, 9H). MS (ESI) m/z 373.3 [M+H]⁺

Compound 11: General procedure A with variant ii) was used for the preparation from compound VI-A employing 2-tert-Butyl-phenyl-isocyanate. ¹H NMR (400 MHz, DMSO-d₆) δ=10.99 (s, 1H), 7.69 (s, 1H), 7.56 (s, 2H), 7.38 (s, 1H), 7.33 (dd, J=1.2, 7.8 Hz, 1H), 7.28-7.23 (m, 1H), 7.18-7.12 (m, 1H), 7.08 (br t, J=6.7 Hz, 2H), 5.12 (dd, J=5.1, 13.3 Hz, 1H), 4.52-4.38 (m, 3H), 4.35-4.27 (m, 1H), 2.98-2.85 (m, 1H), 2.60 (br d, J=17.5 Hz, 1H), 2.40 (br dd, J=4.3, 13.1 Hz, 1H), 2.05-1.96 (m, 1H), 1.35 (s, 9H). MS (ESI) m/z 449.1 [M+H]⁺

Compound 12: General procedure A with variant ii) was used for the preparation from compound VI-A employing iso-Propyl-isocyanate. ¹H NMR (400 MHz, DMSO-d₆) δ=10.98 (s, 1H), 7.60 (s, 1H), 7.52 (q, J=7.7 Hz, 2H), 6.29 (br t, J=6.0 Hz, 1H), 5.82 (d, J=7.8 Hz, 1H), 5.11 (dd, J=5.1, 13.4 Hz, 1H), 4.49-4.37 (m, 1H), 4.34-4.25 (m, 3H), 3.75-3.63 (m, 1H), 2.98-2.83 (m, 1H), 2.60 (br d, J=16.9 Hz, 1H), 2.39 (br dd, J=4.5, 13.1 Hz, 1H), 2.05-1.96 (m, 1H), 1.04 (d, J=6.5 Hz, 6H). MS (ESI) m/z 359.1 [M+H]⁺

Compound 13: General procedure A with variant ii) was used for the preparation from compound VI-A employing n-Hexyl-isocyanate. ¹H NMR (400 MHz, DMSO-d₆) δ=10.98 (s, 1H), 7.60 (s, 1H), 7.54-7.48 (m, 2H), 6.38 (br t, J=5.9 Hz, 1H), 5.95 (br s, 1H), 5.11 (dd, J=5.1, 13.3 Hz, 1H), 4.46-4.38 (m, 1H), 4.33-4.26 (m, 3H), 3.03-2.96 (m, 2H), 2.96-2.86 (m, 1H), 2.60 (br d, J=17.7 Hz, 1H), 2.39 (br dd, J=4.5, 13.1 Hz, 1H), 2.04-1.95 (m, 1H), 1.41-1.32 (m, 2H), 1.24 (br s, 6H), 0.88-0.83 (m, 3H). MS (ESI) m/z 401.1 [M+H]⁺

Compound 14: General procedure A with variant ii) was used for the preparation from compound VI-A employing n-Propyl-isocyanate. ¹H NMR (400 MHz, DMSO-d₆) δ=10.98 (s, 1H), 7.60 (s, 1H), 7.56-7.48 (m, 2H), 6.40 (br t, J=5.9 Hz, 1H), 5.98 (br t, J=5.6 Hz, 1H), 5.11 (dd, J=5.1, 13.3 Hz, 1H), 4.47-4.38 (m, 1H), 4.33-4.26 (m, 3H), 3.00-2.94 (m, 2H), 2.93-2.89 (m, 1H), 2.60 (br d, J=15.7 Hz, 1H), 2.39 (br dd, J=4.5, 13.0 Hz, 1H), 2.06-1.96 (m, 1H), 1.42-1.35 (m, 2H), 0.83 (t, J=7.3 Hz, 3H). MS (ESI) m/z 359.2 [M+H]⁺

Compound 15: General procedure A with variant ii) was used for the preparation from compound VI-A employing Ethyl-isocyanate. ¹H NMR (400 MHz, DMSO-d₆) δ=10.97 (s, 1H), 7.60 (s, 1H), 7.56-7.52 (m, 1H), 7.51-7.47 (m, 1H), 6.41 (t, J=6.1 Hz, 1H), 5.93 (t, J=5.6 Hz, 1H), 5.10 (dd, J=5.1, 13.3 Hz, 1H), 4.46-4.38 (m, 1H), 4.34-4.25 (m, 3H), 3.03 (dq, J=5.7, 7.1 Hz, 2H), 2.91 (ddd, J=5.4, 13.7, 17.5 Hz, 1H), 2.60 (br dd, J=2.2, 15.4 Hz, 1H), 2.45-2.36 (m, 1H), 2.04-1.95 (m, 1H), 1.00 (t, J=7.2 Hz, 3H). MS (ESI) m/z 345.3 [M+H]⁺

Compound 16: General procedure A with variant ii) was used for the preparation from compound VI-A employing 4-Ethoxy-phenyl-isocyanate. ¹H NMR (400 MHz, DMSO-d₆) (=10.98 (br s, 1H), 8.40 (d, J=2.4 Hz, 1H), 7.65 (s, 1H), 7.55 (d, J=1.0 Hz, 2H), 7.33-7.26 (m, 2H), 6.82-6.76 (m, 2H), 6.64 (br d, J=3.3 Hz, 1H), 5.11 (dd, J=5.1, 13.3 Hz, 1H), 4.48-4.34 (m, 3H), 4.33-4.26 (m, 1H), 3.94 (q, J=6.9 Hz, 2H), 2.97-2.85 (m, 1H), 2.59 (td, J=2.0, 15.3 Hz, 1H), 2.45-2.36 (m, 1H), 2.05-1.95 (m, 1H), 1.29 (t, J=7.0 Hz, 3H). MS (ESI) m/z 437.3 [M+H]⁺

Compound 17: General procedure A with variant ii) was used for the preparation from compound VI-A employing 2-Methoxy-5-Methylphenyl-isocyanate. ¹H NMR (400 MHz, DMSO-d₆) δ=10.97 (s, 1H), 7.98 (s, 1H), 7.93 (d, J=2.0 Hz, 1H), 7.65 (s, 1H), 7.56 (s, 2H), 7.40 (br t, J=5.9 Hz, 1H), 6.83 (d, J=8.2 Hz, 1H), 6.67 (dd, J=1.5, 8.3 Hz, 1H), 5.11 (dd, J=5.0, 13.3 Hz, 1H), 4.48-4.36 (m, 3H), 4.35-4.26 (m, 1H), 3.79 (s, 3H), 2.97-2.85 (m, 1H), 2.64-2.56 (m, 1H), 2.39 (br dd, J=4.3, 13.1 Hz, 1H), 2.19 (s, 3H), 2.04-1.96 (m, 1H). MS (ESI) m/z 437.3 [M+H]⁺

Compound 18: General procedure A with variant ii) was used for the preparation from compound VI-A employing 4-Ethylphenyl-isocyanate. ¹H NMR (400 MHz, DMSO-d₆) δ=10.98 (s, 1H), 8.50 (s, 1H), 7.66 (s, 1H), 7.56 (s, 2H), 7.30 (d, J=8.4 Hz, 2H), 7.05 (br d, J=8.4 Hz, 2H), 6.67 (br t, J=5.9 Hz, 1H), 5.11 (dd, J=4.9, 13.2 Hz, 1H), 4.47-4.36 (m, 3H), 4.34-4.26 (m, 1H), 2.97-2.83 (m, 1H), 2.64-2.53 (m, 3H), 2.39 (br dd, J=4.4, 13.3 Hz, 1H), 2.06-1.94 (m, 1H), 1.13 (t, J=7.6 Hz, 3H). MS (ESI) m/z 421.3 [M+H]⁺

Compound 19: General procedure A with variant ii) was used for the preparation from compound VI-A employing 3-Chloro-2-methylphenyl-isocyanate. ¹H NMR (400 MHz, DMSO-d₆) δ=10.99 (br s, 1H), 8.02 (s, 1H), 7.74 (br d, J=7.4 Hz, 1H), 7.68 (s, 1H), 7.57 (s, 2H), 7.16-7.05 (m, 3H), 5.12 (br dd, J=4.5, 13.1 Hz, 1H), 4.50-4.38 (m, 3H), 4.35-4.27 (m, 1H), 2.99-2.82 (m, 1H), 2.62 (br s, 1H), 2.39 (br s, 1H), 2.24 (s, 3H), 2.05-1.95 (m, 1H). MS (ESI) m/z 441.2 [M+H]⁺

Compound 20: General procedure A with variant ii) was used for the preparation from compound VI-A employing 3,5-Dimethylphenyl-isocyanate. ¹H NMR (400 MHz, DMSO-d₆) δ=10.97 (br s, 1H), 8.43 (s, 1H), 7.66 (s, 1H), 7.56 (s, 2H), 7.03 (s, 2H), 6.69 (br t, J=5.9 Hz, 1H), 6.54 (s, 1H), 5.11 (dd, J=5.0, 13.3 Hz, 1H), 4.47-4.36 (m, 3H), 4.34-4.27 (m, 1H), 2.97-2.85 (m, 1H), 2.59 (br dd, J=2.1, 15.3 Hz, 1H), 2.45-2.36 (m, 1H), 2.19 (s, 6H), 2.05-1.95 (m, 1H). MS (ESI) m/z 421.3 [M+H]⁺

Compound 21: General procedure A with variant ii) was used for the preparation from compound VI-A employing 4-Methylphenyl-isocyanate. ¹H NMR (400 MHz, DMSO-d₆) (=10.97 (s, 1H), 8.47 (s, 1H), 7.66 (s, 1H), 7.56 (s, 2H), 7.28 (d, J=8.4 Hz, 2H), 7.02 (d, J=8.2 Hz, 2H), 6.67 (t, J=6.0 Hz, 1H), 5.11 (dd, J=5.0, 13.3 Hz, 1H), 4.46-4.36 (m, 3H), 4.34-4.27 (m, 1H), 2.97-2.85 (m, 1H), 2.63-2.56 (m, 1H), 2.46-2.35 (m, 1H), 2.21 (s, 3H), 2.06-1.95 (m, 1H). MS (ESI) m/z 407.3 [M+H]⁺

Compound 22: General procedure A with variant ii) was used for the preparation from compound VI-A employing 2-Ethoxyphenyl-isocyanate. ¹H NMR (400 MHz, DMSO-d₆) (=8.04 (br d, J=7.3 Hz, 1H), 7.65 (s, 1H), 7.57 (s, 2H), 6.96-6.91 (m, 1H), 6.89-6.79 (m, 2H), 5.07 (br dd, J=4.5, 13.4 Hz, 1H), 4.49-4.36 (m, 3H), 4.35-4.26 (m, 1H), 4.06 (q, J=6.9 Hz, 2H), 2.95-2.82 (m, 1H), 2.65-2.57 (m, 1H), 2.45-2.35 (m, 1H), 2.06-1.94 (m, 1H), 1.36 (t, J=7.0 Hz, 3H). MS (ESI) m/z 437.2 [M+H]⁺

Compound 23: General procedure A with variant ii) was used for the preparation from compound VI-A employing 4-Methoxy-2-methylphenyl-isocyanate. ¹H NMR (400 MHz, DMSO-d₆) δ=10.98 (s, 1H), 7.65 (br d, J=13.2 Hz, 2H), 7.56 (s, 2H), 7.47 (br d, J=8.7 Hz, 1H), 6.84 (br t, J=5.5 Hz, 1H), 6.74 (d, J=2.4 Hz, 1H), 6.68 (dd, J=2.7, 8.7 Hz, 1H), 5.11 (br dd, J=5.0, 13.2 Hz, 1H), 4.49-4.36 (m, 3H), 4.34-4.27 (m, 1H), 3.69 (s, 3H), 2.98-2.84 (m, 1H), 2.60 (br d, J=17.5 Hz, 1H), 2.46-2.36 (m, 1H), 2.16 (s, 3H), 2.05-1.92 (m, 1H). MS (ESI) m/z 437.3 [M+H]⁺

Compound 24: General procedure A with variant ii) was used for the preparation from compound VI-A employing Benzyl-isocyanate. ¹H NMR (400 MHz, DMSO-d₆) δ=¹H NMR (400 MHz, DMSO-d₆) δ=10.97 (s, 1H), 7.64 (s, 1H), 7.56-7.48 (m, 2H), 7.33-7.18 (m, 5H), 6.57 (br t, J=6.1 Hz, 1H), 6.51 (br t, J=5.9 Hz, 1H), 5.12 (dd, J=5.1, 13.3 Hz, 1H), 4.48-4.39 (m, 1H), 4.36-4.26 (m, 3H), 4.24 (d, J=6.0 Hz, 2H), 2.97-2.86 (m, 1H), 2.64-2.56 (m, 1H), 2.46-2.36 (m, 1H), 2.05-1.96 (m, 1H). MS (ESI) m/z 407.1 [M+H]⁺

Compound 25: General procedure A with variant ii) was used for the preparation from compound VI-A employing 2,6-Dimethylphenyl-isocyanate. ¹H NMR (400 MHz, DMSO-d₆) δ=10.98 (s, 1H), 7.68 (s, 1H), 7.61 (s, 1H), 7.55 (s, 2H), 7.07-6.99 (m, 3H), 6.65 (br s, 1H), 5.11 (dd, J=5.1, 13.3 Hz, 1H), 4.48-4.40 (m, 1H), 4.39-4.27 (m, 3H), 2.96-2.86 (m, 1H), 2.60 (br dd, J=2.1, 15.3 Hz, 1H), 2.41 (dt, J=4.4, 13.2 Hz, 1H), 2.17 (s, 6H), 2.06-1.95 (m, 1H). MS (ESI) m/z 421.3 [M+H]⁺

Compound 26: General procedure A with variant ii) was used for the preparation from compound VI-A employing Methylcyclopropyl-isocyanate. ¹H NMR (400 MHz, DMSO-d₆) δ=10.98 (s, 1H), 7.61 (s, 1H), 7.56-7.47 (m, 2H), 6.43 (br t, J=5.9 Hz, 1H), 6.04 (br t, J=5.6 Hz, 1H), 5.11 (dd, J=5.0, 13.2 Hz, 1H), 4.47-4.37 (m, 1H), 4.35-4.25 (m, 3H), 2.90 (br t, J=6.2 Hz, 3H), 2.60 (br d, J=17.2 Hz, 1H), 2.39 (br dd, J=4.3, 13.0 Hz, 1H), 2.05-1.95 (m, 1H), 0.93-0.82 (m, 1H), 0.42-0.33 (m, 2H), 0.13 (br d, J=4.6 Hz, 2H). MS (ESI) m/z 371.3 [M+H]⁺

Compound 27: General procedure A with variant ii) was used for the preparation from compound VI-A employing 2-Phenylethyl-isocyanate. ¹H NMR (400 MHz, DMSO-d₆) (=10.98 (s, 1H), 7.61 (s, 1H), 7.55-7.47 (m, 2H), 7.33-7.26 (m, 2H), 7.23-7.16 (m, 3H), 6.50 (br t, J=5.9 Hz, 1H), 5.99 (br t, J=5.6 Hz, 1H), 5.12 (dd, J=5.1, 13.3 Hz, 1H), 4.47-4.38 (m, 1H), 4.34-4.26 (m, 3H), 3.25 (q, J=6.8 Hz, 2H), 2.97-2.86 (m, 1H), 2.69 (t, J=7.3 Hz, 2H), 2.60 (br d, J=17.5 Hz, 1H), 2.39 (br dd, J=4.4, 13.2 Hz, 1H), 2.04-1.96 (m, 1H). MS (ESI) m/z 421.2 [M+H]⁺

Compound 28: General procedure A with variant ii) was used for the preparation from compound VI-A employing Cyclopentyl-isocyanate. ¹H NMR (400 MHz, DMSO-d₆) (=10.98 (s, 1H), 7.60 (s, 1H), 7.56-7.48 (m, 2H), 6.27 (br t, J=6.0 Hz, 1H), 5.98 (d, J=7.3 Hz, 1H), 5.11 (dd, J=5.1, 13.3 Hz, 1H), 4.46-4.37 (m, 1H), 4.33-4.26 (m, 3H), 3.92-3.81 (m, 1H), 2.97-2.85 (m, 1H), 2.60 (br d, J=17.6 Hz, 1H), 2.39 (br dd, J=4.4, 13.1 Hz, 1H), 2.04-1.96 (m, 1H), 1.84-1.73 (m, 2H), 1.64-1.55 (m, 2H), 1.52-1.44 (m, 2H), 1.35-1.25 (m, 2H). MS (ESI) m/z 385.3 [M+H]⁺

Compound 29: General procedure A with variant ii) was used for the preparation from compound VI-A employing Cyclohexyl-isocyanate. ¹H NMR (400 MHz, DMSO-d₆) δ=10.99 (s, 1H), 7.60 (s, 1H), 7.56-7.47 (m, 2H), 6.30 (br t, J=5.9 Hz, 1H), 5.89 (d, J=8.1 Hz, 1H), 5.11 (dd, J=5.1, 13.3 Hz, 1H), 4.47-4.37 (m, 1H), 4.33-4.25 (m, 3H), 3.44-3.35 (m, 1H), 2.98-2.85 (m, 1H), 2.60 (br d, J=17.6 Hz, 1H), 2.45-2.37 (m, 1H), 2.05-1.94 (m, 1H), 1.81-1.71 (m, 2H), 1.69-1.59 (m, 2H), 1.57-1.47 (m, 1H), 1.32-1.19 (m, 2H), 1.18-1.04 (m, 3H). MS (ESI) m/z 399.3 [M+H]⁺

Compound 30: General procedure A with variant ii) was used for the preparation from compound VI-A employing 5-Chloro-2-methoxyphenyl-isocyanate. ¹H NMR (400 MHz, DMSO-d₆) δ=10.97 (s, 1H), 8.22 (br d, J=15.0 Hz, 2H), 7.66 (s, 1H), 7.61-7.48 (m, 3H), 7.01-6.95 (m, 1H), 6.94-6.87 (m, 1H), 5.12 (dd, J=5.0, 13.2 Hz, 1H), 4.49-4.38 (m, 3H), 4.36-4.26 (m, 1H), 3.84 (s, 3H), 2.97-2.85 (m, 1H), 2.64-2.56 (m, 1H), 2.47-2.36 (m, 1H), 2.07-1.96 (m, 1H). MS (ESI) m/z 457.0 [M+H]⁺

Compound 31: General procedure A with variant ii) was used for the preparation from compound VI-A employing 3-Ethylphenyl-isocyanate. ¹H NMR (400 MHz, DMSO-d₆) δ=10.97 (s, 1H), 8.54 (s, 1H), 7.66 (s, 1H), 7.56 (s, 2H), 7.27 (s, 1H), 7.23-7.17 (m, 1H), 7.15-7.09 (m, 1H), 6.77-6.67 (m, 2H), 5.11 (br dd, J=5.0, 13.2 Hz, 1H), 4.49-4.37 (m, 3H), 4.35-4.27 (m, 1H), 2.98-2.85 (m, 1H), 2.63-2.52 (m, 3H), 2.44-2.36 (m, 1H), 2.05-1.94 (m, 1H), 1.15 (t, J=7.6 Hz, 3H). MS (ESI) m/z 421.0 [M+H]⁺

Compound 32: General procedure A with variant ii) was used for the preparation from compound VI-A employing 4-Chloro-2-Methylphenyl-isocyanate. ¹H NMR (400 MHz, DMSO-d₆) δ=10.97 (br s, 1H), 7.91-7.83 (m, 2H), 7.68 (s, 1H), 7.57 (s, 2H), 7.21 (d, J=2.3 Hz, 1H), 7.19-7.11 (m, 2H), 5.11 (dd, J=5.1, 13.3 Hz, 1H), 4.48-4.38 (m, 3H), 4.35-4.28 (m, 1H), 2.97-2.86 (m, 1H), 2.64-2.56 (m, 1H), 2.44-2.37 (m, 1H), 2.19 (s, 3H), 2.05-1.97 (m, 1H). MS (ESI) m/z 441.0 [M+H]⁺

Compound 33: General procedure A with variant ii) was used for the preparation from compound VI-A employing 3-Chloro-2-Flourophenyl-isocyanate. ¹H NMR (400 MHz, DMSO-d₆) δ=10.97 (s, 1H), 8.63 (br s, 1H), 8.15-8.04 (m, 1H), 7.67 (s, 1H), 7.57 (s, 2H), 7.23 (br t, J=5.7 Hz, 1H), 7.16-7.05 (m, 2H), 5.11 (dd, J=5.1, 13.3 Hz, 1H), 4.50-4.38 (m, 3H), 4.37-4.26 (m, 1H), 2.97-2.85 (m, 1H), 2.60 (br d, J=17.4 Hz, 1H), 2.40 (br dd, J=4.3, 13.2 Hz, 1H), 2.05-1.96 (m, 1H). MS (ESI) m/z 444.9 [M+H]⁺

Compound 34: General procedure A with variant ii) was used for the preparation from compound VI-A employing 3-Flourophenyl-isocyanate. ¹H NMR (400 MHz, DMSO-d₆) (=10.97 (s, 1H), 8.87 (s, 1H), 7.66 (s, 1H), 7.56 (d, J=0.7 Hz, 2H), 7.45 (td, J=2.3, 12.2 Hz, 1H), 7.29-7.20 (m, 1H), 7.06 (dd, J=1.2, 8.1 Hz, 1H), 6.83 (t, J=5.9 Hz, 1H), 6.70 (dt, J=2.0, 8.5 Hz, 1H), 5.11 (dd, J=5.0, 13.2 Hz, 1H), 4.48-4.38 (m, 3H), 4.35-4.27 (m, 1H), 2.97-2.85 (m, 1H), 2.59 (br dd, J=2.1, 15.5 Hz, 1H), 2.39 (br dd, J=4.4, 13.0 Hz, 1H), 2.04-1.96 (m, 1H). MS (ESI) m/z 411.0 [M+H]⁺

Compound 35: General procedure A with variant ii) was used for the preparation from compound VI-A employing 2,5-Dichlorophenyl-isocyanate. ¹H NMR (400 MHz, DMSO-d₆) δ=10.97 (br s, 1H), 8.32 (s, 2H), 7.74-7.64 (m, 2H), 7.58 (s, 2H), 7.44 (d, J=8.6 Hz, 1H), 7.02 (dd, J=2.4, 8.6 Hz, 1H), 5.12 (dd, J=5.1, 13.3 Hz, 1H), 4.49-4.40 (m, 3H), 4.37-4.27 (m, 1H), 2.97-2.85 (m, 1H), 2.60 (br d, J=17.5 Hz, 1H), 2.40 (br dd, J=4.2, 13.1 Hz, 1H), 2.07-1.95 (m, 1H). MS (ESI) m/z 460.9 [M+H]⁺

Compound 36: General procedure A with variant ii) was used for the preparation from compound VI-A employing 2-Ethylphenyl-isocyanate. ¹H NMR (400 MHz, DMSO-d₆) δ=10.97 (br s, 1H), 7.76 (br d, J=6.7 Hz, 2H), 7.68 (s, 1H), 7.57 (s, 2H), 7.17-7.06 (m, 3H), 6.94 (dt, J=1.1, 7.4 Hz, 1H), 5.12 (dd, J=5.1, 13.2 Hz, 1H), 4.48-4.37 (m, 3H), 4.35-4.26 (m, 1H), 2.96-2.85 (m, 1H), 2.64-2.58 (m, 1H), 2.58-2.53 (m, 2H), 2.46-2.37 (m, 1H), 2.05-1.95 (m, 1H), 1.14 (t, J=7.5 Hz, 3H). MS (ESI) m/z 421.3 [M+H]⁺

Compound 37: General procedure A with variant ii) was used for the preparation from compound VI-A employing 2-Methylphenyl-isocyanate. ¹H NMR (400 MHz, DMSO-d₆) (=10.97 (br s, 1H), 7.84-7.76 (m, 2H), 7.69 (s, 1H), 7.57 (s, 2H), 7.16-7.05 (m, 3H), 6.88 (dt, J=0.9, 7.4 Hz, 1H), 5.12 (dd, J=5.1, 13.3 Hz, 1H), 4.49-4.38 (m, 3H), 4.36-4.26 (m, 1H), 2.98-2.85 (m, 1H), 2.64-2.55 (m, 1H), 2.47-2.36 (m, 1H), 2.19 (s, 3H), 2.05-1.96 (m, 1H). MS (ESI) m/z 407.3 [M+H]⁺

Compound 38: General procedure A with variant ii) was used for the preparation from compound VI-A employing 3,5-Dichlorophenyl-isocyanate. ¹H NMR (400 MHz, DMSO-d₆) δ=10.97 (br s, 1H), 9.13 (s, 1H), 7.66 (s, 1H), 7.56 (d, J=0.7 Hz, 2H), 7.50 (d, J=1.8 Hz, 2H), 7.07 (br d, J=1.7 Hz, 2H), 5.11 (dd, J=5.1, 13.3 Hz, 1H), 4.48-4.36 (m, 3H), 4.35-4.27 (m, 1H), 2.97-2.83 (m, 1H), 2.64-2.56 (m, 1H), 2.45-2.37 (m, 1H), 2.04-1.96 (m, 1H). MS (ESI) m/z 461.2 [M+H]⁺

Compound 39: General procedure A with variant ii) was used for the preparation from compound VI-A employing 3,4-Diflourophenyl-isocyanate. ¹H NMR (400 MHz, DMSO-d₆) δ=10.97 (s, 1H), 8.90 (s, 1H), 7.66 (s, 1H), 7.62 (dt, J=2.6, 6.8 Hz, 1H), 7.56 (d, J=0.9 Hz, 2H), 7.33-7.22 (m, 1H), 7.09-7.03 (m, 1H), 6.93-6.82 (m, 1H), 5.11 (dd, J=5.1, 13.3 Hz, 1H), 4.48-4.37 (m, 3H), 4.34-4.26 (m, 1H), 2.98-2.85 (m, 1H), 2.64-2.56 (m, 1H), 2.46-2.36 (m, 1H), 2.04-1.95 (m, 1H). MS (ESI) m/z 429.2 [M+H]⁺

Compound 40: General procedure A with variant ii) was used for the preparation from compound VI-A employing 1,2,3,4-Tetrahydronaphth-1-yl-isocyanate. ¹H NMR (400 MHz, DMSO-d₆) δ=10.98 (s, 1H), 7.65 (s, 1H), 7.57-7.51 (m, 2H), 7.26-7.20 (m, 1H), 7.17-7.11 (m, 2H), 7.09-7.03 (m, 1H), 6.41-6.29 (m, 2H), 5.12 (dd, J=5.1, 13.3 Hz, 1H), 4.85-4.77 (m, 1H), 4.47-4.40 (m, 1H), 4.39-4.28 (m, 3H), 2.97-2.85 (m, 1H), 2.77-2.66 (m, 2H), 2.64-2.57 (m, 1H), 2.40 (dd, J=4.6, 13.1 Hz, 1H), 2.06-1.97 (m, 1H), 1.92-1.64 (m, 4H). MS (ESI) m/z 447.3 [M+H]⁺

Compound 41: General procedure A with variant ii) was used for the preparation from compound VI-A employing 4-Triflouromethylphenyl-isocyanate. ¹H NMR (400 MHz, DMSO-d₆) δ=10.98 (s, 1H), 9.26-9.05 (m, 1H), 7.70-7.61 (m, 3H), 7.61-7.54 (m, 4H), 7.00 (br d, J=6.1 Hz, 1H), 5.12 (dd, J=5.1, 13.3 Hz, 1H), 4.52-4.38 (m, 3H), 4.37-4.27 (m, 1H), 2.92 (ddd, J=5.4, 13.6, 17.4 Hz, 1H), 2.65-2.57 (m, 1H), 2.47-2.36 (m, 1H), 2.10-1.95 (m, 1H). MS (ESI) m/z 461.0 [M+H]⁺

Compound 42: General procedure A with variant ii) was used for the preparation from compound VI-A employing 3-Chloro-4-Fluorophenyl-isocyanate. ¹H NMR (400 MHz, DMSO-d₆) δ=10.97 (s, 1H), 8.85 (s, 1H), 7.80-7.72 (m, 1H), 7.66 (s, 1H), 7.56 (d, J=0.9 Hz, 2H), 7.31-7.21 (m, 2H), 6.86 (t, J=5.9 Hz, 1H), 5.11 (dd, J=5.1, 13.3 Hz, 1H), 4.49-4.37 (m, 3H), 4.35-4.26 (m, 1H), 2.98-2.84 (m, 1H), 2.60 (br d, J=17.6 Hz, 1H), 2.45-2.36 (m, 1H), 2.05-1.95 (m, 1H). MS (ESI) m/z 445.2 [M+H]⁺

Compound 43: General procedure A with variant ii) was used for the preparation from compound VI-A employing 3-Methylphenyl-isocyanate. ¹H NMR (400 MHz, DMSO-d₆) (=10.97 (br s, 1H), 8.62-8.50 (m, 1H), 7.66 (s, 1H), 7.56 (s, 2H), 7.25 (s, 1H), 7.18 (br d, J=8.3 Hz, 1H), 7.14-7.05 (m, 1H), 6.81-6.68 (m, 2H), 5.11 (dd, J=5.1, 13.3 Hz, 1H), 4.49-4.36 (m, 3H), 4.35-4.26 (m, 1H), 2.97-2.85 (m, 1H), 2.64-2.56 (m, 1H), 2.45-2.36 (m, 1H), 2.24 (s, 3H), 2.06-1.95 (m, 1H). MS (ESI) m/z 407.2 [M+H]⁺

Compound 44: General procedure A with variant ii) was used for the preparation from compound VI-A employing 2,3-Dimethylphenyl-isocyanate. ¹H NMR (400 MHz, DMSO-d₆) δ=10.97 (s, 1H), 7.80 (s, 1H), 7.68 (s, 1H), 7.57 (s, 2H), 7.50 (d, J=8.1 Hz, 1H), 7.03-6.94 (m, 2H), 6.84 (d, J=7.2 Hz, 1H), 5.11 (dd, J=5.1, 13.3 Hz, 1H), 4.49-4.37 (m, 3H), 4.36-4.27 (m, 1H), 2.98-2.85 (m, 1H), 2.64-2.57 (m, 1H), 2.46-2.37 (m, 1H), 2.23 (s, 3H), 2.08 (s, 3H), 2.04-1.97 (m, 1H). MS (ESI) m/z 421.3 [M+H]⁺

Compound 45: General procedure A with variant ii) was used for the preparation from compound VI-A employing 2,4-Dichlorophenyl-isocyanate. ¹H NMR (400 MHz, DMSO-d₆) δ=10.97 (s, 1H), 8.26 (s, 1H), 8.19 (d, J=8.9 Hz, 1H), 7.68 (s, 1H), 7.58 (s, 3H), 7.56 (d, J=2.4 Hz, 1H), 7.32 (dd, J=2.5, 9.0 Hz, 1H), 5.11 (dd, J=5.1, 13.4 Hz, 1H), 4.51-4.40 (m, 3H), 4.37-4.27 (m, 1H), 2.98-2.85 (m, 1H), 2.64-2.56 (m, 1H), 2.46-2.37 (m, 1H), 2.06-1.97 (m, 1H). MS (ESI) m/z 460.9 [M+H]⁺

Compound 46: General procedure A with variant ii) was used for the preparation from compound VI-A employing 3,4-Dichlorophenyl-isocyanate. ¹H NMR (400 MHz, DMSO-d₆) δ=10.98 (s, 1H), 9.08 (br s, 1H), 7.85 (d, J=2.4 Hz, 1H), 7.66 (s, 1H), 7.56 (s, 2H), 7.45 (d, J=8.8 Hz, 1H), 7.28 (dd, J=2.4, 8.8 Hz, 1H), 7.02 (br d, J=5.7 Hz, 1H), 5.11 (dd, J=5.0, 13.3 Hz, 1H), 4.48-4.36 (m, 3H), 4.34-4.27 (m, 1H), 2.97-2.85 (m, 1H), 2.59 (br d, J=17.6 Hz, 1H), 2.42-2.38 (m, 1H), 2.04-1.96 (m, 1H). MS (ESI) m/z 460.9 [M+H]⁺

Compound 47: General procedure A with variant ii) was used for the preparation from compound VI-A employing 3-Trifluoromethylphenyl-isocyanate. ¹H NMR (400 MHz, DMSO-d₆) δ=10.98 (s, 1H), 9.08 (s, 1H), 7.98 (s, 1H), 7.67 (s, 1H), 7.57 (s, 2H), 7.54 (br d, J=8.8 Hz, 1H), 7.45 (t, J=7.9 Hz, 1H), 7.23 (d, J=7.5 Hz, 1H), 6.95 (br t, J=5.9 Hz, 1H), 5.11 (dd, J=5.1, 13.3 Hz, 1H), 4.48-4.37 (m, 3H), 4.35-4.26 (m, 1H), 2.98-2.84 (m, 1H), 2.59 (br d, J=17.5 Hz, 1H), 2.39 (br dd, J=4.5, 13.1 Hz, 1H), 2.04-1.95 (m, 1H). MS (ESI) m/z 461.2 [M+H]⁺

Compound 48: General procedure A with variant ii) was used for the preparation from compound VI-A employing 2-Fluorophenyl-isocyanate. ¹H NMR (400 MHz, DMSO-d₆) (=10.99 (s, 1H), 8.47 (br s, 1H), 8.16-8.08 (m, 1H), 7.66 (s, 1H), 7.57 (s, 2H), 7.24-7.14 (m, 2H), 7.08 (t, J=7.6 Hz, 1H), 6.97-6.90 (m, 1H), 5.12 (dd, J=5.1, 13.3 Hz, 1H), 4.50-4.38 (m, 3H), 4.36-4.25 (m, 1H), 2.98-2.84 (m, 1H), 2.62-2.57 (m, 1H), 2.44-2.36 (m, 1H), 2.05-1.95 (m, 1H). MS (ESI) m/z 411.2 [M+H]⁺

Compound 49: General procedure A with variant ii) was used for the preparation from compound VI-A employing 2-Triflouromethylphenyl-isocyanate. ¹H NMR (400 MHz, DMSO-d₆) δ=10.99 (s, 1H), 8.00-7.90 (m, 2H), 7.68 (s, 1H), 7.62 (d, J=7.9 Hz, 1H), 7.60-7.55 (m, 4H), 7.20 (t, J=7.6 Hz, 1H), 5.12 (dd, J=5.1, 13.3 Hz, 1H), 4.49-4.39 (m, 3H), 4.35-4.27 (m, 1H), 2.97-2.85 (m, 1H), 2.64-2.56 (m, 1H), 2.42-2.37 (m, 1H), 2.05-1.96 (m, 1H). MS (ESI) m/z 461.2 [M+H]⁺

Compound 50: General procedure A with variant ii) was used for the preparation from compound VI-A employing 2,3-Dichlorophenyl-isocyanate. ¹H NMR (400 MHz, DMSO-d₆) δ=10.97 (s, 1H), 8.33 (s, 1H), 8.16 (dd, J=1.5, 8.2 Hz, 1H), 7.72-7.60 (m, 2H), 7.58 (s, 2H), 7.32-7.16 (m, 2H), 5.12 (dd, J=5.0, 13.2 Hz, 1H), 4.50-4.37 (m, 3H), 4.36-4.28 (m, 1H), 2.97-2.85 (m, 1H), 2.60 (br dd, J=2.1, 15.5 Hz, 1H), 2.40 (br dd, J=4.5, 13.1 Hz, 1H), 2.05-1.94 (m, 1H). MS (ESI) m/z 461.1 [M+H]⁺

Compound 51: General procedure A with variant ii) was used for the preparation from compound VI-A employing 2-(4-Methoxyphenyl)-ethyl-isocyanate. ¹H NMR (400 MHz, DMSO-d₆) δ=10.99 (s, 1H), 7.61 (s, 1H), 7.56-7.45 (m, 2H), 7.11 (br d, J=8.6 Hz, 2H), 6.85 (d, J=8.6 Hz, 2H), 6.49 (br t, J=5.7 Hz, 1H), 5.96 (br t, J=5.6 Hz, 1H), 5.11 (dd, J=5.0, 13.3 Hz, 1H), 4.48-4.37 (m, 1H), 4.35-4.24 (m, 3H), 3.71 (s, 3H), 3.25-3.16 (m, 2H), 2.98-2.84 (m, 1H), 2.65-2.55 (m, 3H), 2.39 (br dd, J=4.3, 13.0 Hz, 1H), 2.05-1.95 (m, 1H). MS (ESI) m/z 451.0 [M+H]⁺

Compound 52: General procedure A with variant ii) was used for the preparation from compound VI-A employing 4-Methoxyphenylmethyl-isocyanate. ¹H NMR (400 MHz, DMSO-d₆) δ=10.99 (s, 1H), 7.63 (s, 1H), 7.57-7.47 (m, 2H), 7.17 (d, J=8.6 Hz, 2H), 6.87 (d, J=8.7 Hz, 2H), 6.52 (br t, J=6.0 Hz, 1H), 6.42 (br t, J=5.9 Hz, 1H), 5.12 (dd, J=5.0, 13.3 Hz, 1H), 4.47-4.38 (m, 1H), 4.36-4.26 (m, 3H), 4.16 (d, J=5.9 Hz, 2H), 3.72 (s, 3H), 2.98-2.84 (m, 1H), 2.64-2.56 (m, 1H), 2.44-2.36 (m, 1H), 2.05-1.94 (m, 1H). MS (ESI) m/z 437.3 [M+H]⁺

Compound 53: General procedure A with variant ii) was used for the preparation from compound VI-A employing 3-Methoxyphenylmethyl-isocyanate. ¹H NMR (400 MHz, DMSO-d₆) δ=10.99 (s, 1H), 7.64 (s, 1H), 7.57-7.47 (m, 2H), 7.22 (t, J=7.8 Hz, 1H), 6.86-6.75 (m, 3H), 6.58 (br t, J=6.1 Hz, 1H), 6.50 (br t, J=6.0 Hz, 1H), 5.12 (dd, J=5.1, 13.3 Hz, 1H), 4.49-4.38 (m, 1H), 4.36-4.26 (m, 3H), 4.21 (d, J=5.9 Hz, 2H), 3.71 (s, 3H), 2.98-2.85 (m, 1H), 2.64-2.56 (m, 1H), 2.40 (br dd, J=4.4, 13.1 Hz, 1H), 2.05-1.95 (m, 1H). MS (ESI) m/z 437.3 [M+H]⁺

Compound 54: General procedure A with variant ii) was used for the preparation from compound VI-A employing (R)-1-Phenylethyl-isocyanate. ¹H NMR (400 MHz, DMSO-d₆) δ=10.99 (s, 1H), 7.61 (s, 1H), 7.55-7.45 (m, 2H), 7.34-7.27 (m, 4H), 7.20 (dt, J=2.6, 5.7 Hz, 1H), 6.50 (d, J=8.1 Hz, 1H), 6.41 (br t, J=5.9 Hz, 1H), 5.11 (dd, J=5.1, 13.3 Hz, 1H), 4.76 (quin, J=7.3 Hz, 1H), 4.47-4.38 (m, 1H), 4.34-4.25 (m, 3H), 2.98-2.85 (m, 1H), 2.63-2.56 (m, 1H), 2.39 (br dd, J=4.5, 13.2 Hz, 1H), 2.05-1.95 (m, 1H), 1.33 (d, J=7.0 Hz, 3H). MS (ESI) m/z 421.0 [M+H]⁺

Compound 55: General procedure A with variant ii) was used for the preparation from compound VI-A employing (S)-1-Phenylethyl-isocyanate. ¹H NMR (400 MHz, DMSO-d₆) δ=10.99 (s, 1H), 7.61 (s, 1H), 7.55-7.45 (m, 2H), 7.34-7.26 (m, 4H), 7.20 (dt, J=2.7, 5.6 Hz, 1H), 6.50 (d, J=8.1 Hz, 1H), 6.40 (br t, J=5.9 Hz, 1H), 5.11 (dd, J=5.0, 13.3 Hz, 1H), 4.76 (quin, J=7.2 Hz, 1H), 4.46-4.38 (m, 1H), 4.35-4.23 (m, 3H), 2.98-2.85 (m, 1H), 2.60 (br d, J=17.6 Hz, 1H), 2.39 (br dd, J=4.4, 13.1 Hz, 1H), 2.05-1.95 (m, 1H), 1.33 (d, J=7.0 Hz, 3H). MS (ESI) m/z 421.0 [M+H]⁺

Compound 56: General procedure A with variant ii) was used for the preparation from compound VI-A employing 4-tert-Butylphenyl-isocyanate. ¹H NMR (400 MHz, DMSO-d₆) δ=10.98 (s, 1H), 8.52 (s, 1H), 7.66 (s, 1H), 7.56 (d, J=0.9 Hz, 2H), 7.34-7.27 (m, 2H), 7.26-7.20 (m, 2H), 6.68 (t, J=6.0 Hz, 1H), 5.11 (dd, J=5.1, 13.3 Hz, 1H), 4.48-4.34 (m, 3H), 4.34-4.26 (m, 1H), 2.96-2.84 (m, 1H), 2.59 (td, J=1.9, 15.4 Hz, 1H), 2.43-2.36 (m, 1H), 2.04-1.94 (m, 1H), 2.04-1.94 (m, 1H), 1.24 (s, 9H). MS (ESI) m/z 449.3 [M+H]⁺

Compound 57: General procedure A with variant ii) was used for the preparation from compound VI-A employing 4-iso-Propylphenyl-isocyanate. ¹H NMR (400 MHz, DMSO-d₆) δ=10.98 (s, 1H), 8.50 (s, 1H), 7.66 (s, 1H), 7.56 (s, 2H), 7.31 (d, J=8.6 Hz, 2H), 7.09 (d, J=8.6 Hz, 2H), 6.67 (br t, J=5.9 Hz, 1H), 5.11 (dd, J=5.0, 13.3 Hz, 1H), 4.52-4.37 (m, 3H), 4.35-4.25 (m, 1H), 2.97-2.85 (m, 1H), 2.84-2.74 (m, 1H), 2.59 (br d, J=17.5 Hz, 1H), 2.46-2.36 (m, 1H), 2.05-1.92 (m, 1H), 1.16 (d, J=6.8 Hz, 6H). MS (ESI) m/z 435.3 [M+H]⁺

Compound 58: General procedure A with variant ii) was used for the preparation from compound VI-A employing Cyclohexylmethyl-isocyanate. ¹H NMR (400 MHz, DMSO-d₆) δ=10.98 (s, 1H), 7.60 (s, 1H), 7.51 (q, J=7.7 Hz, 2H), 6.36 (br t, J=6.1 Hz, 1H), 6.00 (t, J=5.7 Hz, 1H), 5.11 (dd, J=5.1, 13.3 Hz, 1H), 4.48-4.37 (m, 1H), 4.34-4.25 (m, 3H), 2.98-2.83 (m, 3H), 2.63-2.56 (m, 1H), 2.44-2.36 (m, 1H), 2.06-1.92 (m, 1H), 1.74-1.56 (m, 5H), 1.42-1.25 (m, 1H), 1.24-1.07 (m, 3H), 0.91-0.78 (m, 2H). MS (ESI) m/z 413.3 [M+H]⁺

Compound 59: General procedure A with variant ii) was used for the preparation from compound VI-A employing 3-Chloro-4-methoxyphenyl-isocyanate. ¹H NMR (400 MHz, DMSO-d₆) δ=10.97 (br s, 1H), 8.73-8.59 (m, 1H), 7.68-7.60 (m, 2H), 7.56 (s, 2H), 7.21 (dd, J=2.3, 8.9 Hz, 1H), 7.02 (d, J=8.9 Hz, 1H), 6.80 (br d, J=3.4 Hz, 1H), 5.11 (dd, J=5.0, 13.3 Hz, 1H), 4.49-4.35 (m, 3H), 4.34-4.26 (m, 1H), 3.78 (s, 3H), 2.97-2.84 (m, 1H), 2.59 (br d, J=17.1 Hz, 1H), 2.39 (br dd, J=4.2, 13.0 Hz, 1H), 2.05-1.95 (m, 1H). MS (ESI) m/z 457.2 [M+H]⁺

Compound 60: General procedure A with variant ii) was used for the preparation from compound VI-A employing 2-Methoxyphenylmethyl-isocyanate. ¹H NMR (400 MHz, DMSO-d₆) δ=10.98 (br s, 1H), 7.63 (s, 1H), 7.57-7.48 (m, 2H), 7.25-7.15 (m, 2H), 6.96 (d, J=8.1 Hz, 1H), 6.90 (t, J=7.3 Hz, 1H), 6.60 (t, J=6.0 Hz, 1H), 6.31 (t, J=5.9 Hz, 1H), 5.12 (dd, J=5.1, 13.4 Hz, 1H), 4.47-4.38 (m, 1H), 4.36-4.26 (m, 3H), 4.19 (d, J=6.0 Hz, 2H), 3.80 (s, 3H), 3.01-2.83 (m, 1H), 2.60 (br d, J=17.6 Hz, 1H), 2.40 (br dd, J=4.4, 13.1 Hz, 1H), 2.04-1.96 (m, 1H). MS (ESI) m/z 437.3 [M+H]⁺

Compound 61: General procedure A with variant ii) was used for the preparation from compound VI-A employing 4-Chloro-3-methylphenyl-isocyanate. ¹H NMR (400 MHz, DMSO-d₆) δ=10.98 (s, 1H), 8.71 (s, 1H), 7.66 (s, 1H), 7.56 (s, 2H), 7.40 (d, J=1.8 Hz, 1H), 7.30-7.25 (m, 1H), 7.24-7.19 (m, 1H), 6.87-6.75 (m, 1H), 5.11 (dd, J=5.0, 13.3 Hz, 1H), 4.48-4.35 (m, 3H), 4.34-4.25 (m, 1H), 2.98-2.84 (m, 1H), 2.60 (br dd, J=1.6, 17.4 Hz, 1H), 2.46-2.36 (m, 1H), 2.26 (s, 3H), 2.06-1.94 (m, 1H). MS (ESI) m/z 441.3 [M+H]⁺

Compound 62: General procedure A with variant ii) was used for the preparation from compound VI-A employing 5-Fluoro-2-methylphenyl-isocyanate. ¹H NMR (400 MHz, DMSO-d₆) δ=10.98 (s, 1H), 7.96-7.79 (m, 2H), 7.68 (s, 1H), 7.58 (s, 2H), 7.31 (t, J=5.8 Hz, 1H), 7.12 (t, J=7.6 Hz, 1H), 6.66 (dt, J=2.8, 8.3 Hz, 1H), 5.12 (dd, J=5.1, 13.3 Hz, 1H), 4.51-4.36 (m, 3H), 4.36-4.27 (m, 1H), 3.02-2.80 (m, 1H), 2.65-2.56 (m, 1H), 2.46-2.36 (m, 1H), 2.16 (s, 3H), 2.05-1.95 (m, 1H). MS (ESI) m/z 425.3 [M+H]⁺

Compound 63: General procedure A with variant ii) was used for the preparation from compound VI-A employing 3-Methylphenylmethyl-isocyanate. ¹H NMR (400 MHz, DMSO-d₆) δ=10.98 (s, 1H), 7.64 (s, 1H), 7.57-7.49 (m, 2H), 7.23-7.15 (m, 1H), 7.11-6.96 (m, 3H), 6.56 (br t, J=5.9 Hz, 1H), 6.48 (br t, J=5.9 Hz, 1H), 5.12 (dd, J=5.1, 13.3 Hz, 1H), 4.48-4.39 (m, 1H), 4.36-4.27 (m, 3H), 4.20 (d, J=6.0 Hz, 2H), 2.98-2.84 (m, 1H), 2.60 (br dd, J=1.6, 17.4 Hz, 1H), 2.44-2.36 (m, 1H), 2.27 (s, 3H), 2.05-1.96 (m, 1H). MS (ESI) m/z 421.3 [M+H]⁺

Compound 64: General procedure A with variant ii) was used for the preparation from compound VI-A employing 2-(4-Chlorophenyl)-ethyl-isocyanate. ¹H NMR (400 MHz, DMSO-d₆) δ=10.98 (s, 1H), 7.61 (s, 1H), 7.57-7.51 (m, 1H), 7.50-7.45 (m, 1H), 7.34 (d, J=8.3 Hz, 2H), 7.22 (d, J=8.4 Hz, 2H), 6.48 (br t, J=6.0 Hz, 1H), 5.99 (t, J=5.6 Hz, 1H), 5.12 (dd, J=5.1, 13.3 Hz, 1H), 4.48-4.37 (m, 1H), 4.35-4.25 (m, 3H), 3.24 (q, J=6.8 Hz, 2H), 2.98-2.85 (m, 1H), 2.69 (t, J=7.0 Hz, 2H), 2.65-2.56 (m, 1H), 2.45-2.37 (m, 1H), 2.05-1.94 (m, 1H). MS (ESI) m/z 455.3 [M+H]⁺

Compound 65: General procedure A with variant ii) was used for the preparation from compound VI-A employing 4-Trifluoromethoylphenylmethyl-isocyanate. ¹H NMR (400 MHz, DMSO-d₆) δ=10.98 (s, 1H), 7.71-7.62 (m, 3H), 7.56-7.50 (m, 2H), 7.47 (br d, J=8.1 Hz, 2H), 6.74-6.60 (m, 2H), 5.12 (dd, J=5.1, 13.3 Hz, 1H), 4.48-4.39 (m, 1H), 4.37-4.27 (m, 5H), 2.99-2.85 (m, 1H), 2.65-2.56 (m, 1H), 2.45-2.36 (m, 1H), 2.05-1.95 (m, 1H). MS (ESI) m/z 475.3 [M+H]⁺

Compound 66: General procedure A with variant ii) was used for the preparation from compound VI-A employing 2-Biphenyl-isocyanate. ¹H NMR (400 MHz, DMSO-d₆) δ=10.98 (s, 1H), 7.85 (d, J=7.9 Hz, 1H), 7.63 (s, 1H), 7.58-7.54 (m, 1H), 7.53-7.46 (m, 4H), 7.42-7.34 (m, 3H), 7.31-7.25 (m, 1H), 7.17 (dd, J=1.4, 7.5 Hz, 1H), 7.15-7.06 (m, 2H), 5.12 (dd, J=5.0, 13.3 Hz, 1H), 4.49-4.39 (m, 1H), 4.38-4.26 (m, 3H), 2.98-2.85 (m, 1H), 2.64-2.56 (m, 1H), 2.40 (br dd, J=4.5, 13.1 Hz, 1H), 2.05-1.94 (m, 1H). MS (ESI) m/z 469.3 [M+H]⁺

Compound 67: General procedure A with variant ii) was used for the preparation from compound VI-A employing 2-Fluoro-5-methylphenyl-isocyanate. ¹H NMR (400 MHz, DMSO-d₆) δ=10.98 (s, 1H), 8.36 (d, J=2.1 Hz, 1H), 7.95 (dd, J=1.6, 7.9 Hz, 1H), 7.66 (s, 1H), 7.57 (s, 2H), 7.16 (t, J=5.8 Hz, 1H), 7.04 (dd, J=8.3, 11.5 Hz, 1H), 6.77-6.67 (m, 1H), 5.11 (dd, J=5.1, 13.3 Hz, 1H), 4.48-4.39 (m, 3H), 4.35-4.28 (m, 1H), 2.97-2.86 (m, 1H), 2.64-2.56 (m, 1H), 2.39 (dq, J=5.0, 13.4 Hz, 1H), 2.23 (s, 3H), 2.04-1.96 (m, 1H). MS (ESI) m/z 425.0 [M+H]⁺

Compound 68: General procedure A with variant ii) was used for the preparation from compound VI-A employing 3,4-Dimethylphenyl-isocyanate. ¹H NMR (400 MHz, DMSO-d₆) δ=10.98 (s, 1H), 8.40 (s, 1H), 7.65 (s, 1H), 7.56 (s, 2H), 7.18 (s, 1H), 7.11 (dd, J=2.0, 8.0 Hz, 1H), 6.96 (d, J=8.2 Hz, 1H), 6.66 (t, J=5.9 Hz, 1H), 5.11 (dd, J=5.0, 13.2 Hz, 1H), 4.49-4.26 (m, 4H), 2.96-2.86 (m, 1H), 2.63-2.56 (m, 1H), 2.45-2.33 (m, 1H), 2.15 (s, 3H), 2.12 (s, 3H), 2.04-1.96 (m, 1H). MS (ESI) m/z 421.0 [M+H]⁺

Compound 69: General procedure A with variant ii) was used for the preparation from compound VI-A employing 2,5-Difluorophenyl-isocyanate. ¹H NMR (400 MHz, DMSO-d₆) δ=10.98 (s, 1H), 8.69 (br s, 1H), 8.02 (ddd, J=3.2, 6.6, 11.3 Hz, 1H), 7.66 (s, 1H), 7.57 (s, 2H), 7.34-7.18 (m, 2H), 6.83-6.67 (m, 1H), 5.11 (dd, J=5.1, 13.3 Hz, 1H), 4.49-4.39 (m, 3H), 4.36-4.27 (m, 1H), 2.98-2.85 (m, 1H), 2.64-2.56 (m, 1H), 2.45-2.34 (m, 1H), 2.05-1.95 (m, 1H). MS (ESI) m/z 429.0 [M+H]⁺

Compound 70: General procedure A with variant ii) was used for the preparation from compound VI-A employing 3-Phenyl-n-propyl-isocyanate. ¹H NMR (400 MHz, DMSO-d₆) δ=10.98 (s, 1H), 7.62 (s, 1H), 7.56-7.47 (m, 2H), 7.31-7.24 (m, 2H), 7.22-7.12 (m, 3H), 6.44 (br t, J=6.1 Hz, 1H), 6.07 (t, J=5.6 Hz, 1H), 5.11 (dd, J=5.1, 13.3 Hz, 1H), 4.46-4.38 (m, 1H), 4.35-4.25 (m, 3H), 3.02 (q, J=6.6 Hz, 2H), 2.96-2.86 (m, 1H), 2.64-2.58 (m, 1H), 2.58-2.54 (m, 2H), 2.45-2.33 (m, 1H), 2.05-1.95 (m, 1H), 1.67 (quin, J=7.3 Hz, 2H). MS (ESI) m/z 435.0 [M+H]⁺

Compound 71: General procedure A with variant ii) was used for the preparation from compound VI-A employing 4-Biphenyl-isocyanate. ¹H NMR (400 MHz, DMSO-d₆) δ=10.98 (s, 1H), 8.77 (s, 1H), 7.68 (s, 1H), 7.63-7.49 (m, 8H), 7.42 (t, J=7.7 Hz, 2H), 7.32-7.27 (m, 1H), 6.82 (br t, J=5.9 Hz, 1H), 5.11 (dd, J=5.1, 13.3 Hz, 1H), 4.50-4.37 (m, 3H), 4.35-4.27 (m, 1H), 2.97-2.85 (m, 1H), 2.64-2.57 (m, 1H), 2.45-2.34 (m, 1H), 2.05-1.95 (m, 1H). MS (ESI) m/z 469.0 [M+H]⁺

Compound 72: General procedure A with variant ii) was used for the preparation from compound VI-A employing 2-Methoxyethyl-isocyanate. ¹H NMR (400 MHz, DMSO-d₆) δ=10.96 (br s, 1H), 7.60 (s, 1H), 7.56-7.45 (m, 2H), 6.52 (br t, J=5.7 Hz, 1H), 6.05 (br t, J=5.3 Hz, 1H), 5.11 (br dd, J=5.0, 13.2 Hz, 1H), 4.47-4.38 (m, 1H), 4.35-4.25 (m, 3H), 3.32-3.31 (m, 2H), 3.25 (s, 3H), 3.18 (q, J=5.5 Hz, 2H), 2.97-2.85 (m, 1H), 2.63-2.56 (m, 1H), 2.45-2.33 (m, 1H), 2.05-1.94 (m, 1H). MS (ESI) m/z 375.0 [M+H]⁺.

Compound 73: General procedure with variant ii) was used for the preparation from compound VI-A employing 3-isocyanatobenzonitrile. ¹H NMR (400 MHz, DMSO-d₆) δ=10.98 (s, 1H), 9.10 (s, 1H), 7.95 (t, J=1.7 Hz, 1H), 7.67 (s, 1H), 7.64-7.59 (m, 1H), 7.56 (s, 2H), 7.43 (t, J=7.9 Hz, 1H), 7.34 (td, J=1.2, 7.7 Hz, 1H), 7.04 (br t, J=5.9 Hz, 1H), 5.11 (dd, J=5.1, 13.3 Hz, 1H), 4.48-4.37 (m, 3H), 4.34-4.27 (m, 1H), 2.97-2.84 (m, 1H), 2.62-2.57 (m, 1H), 2.43-2.29 (m, 1H), 2.05-1.94 (m, 1H). MS (ESI) m/z 418.2 [M+H]⁺

Compound 74: General procedure with variant ii) was used for the preparation from compound VI-A employing 4-isocyanatobenzonitrile. ¹H NMR (400 MHz, DMSO-d₆) δ=10.97 (br s, 1H), 9.23 (s, 1H), 7.70-7.63 (m, 3H), 7.62-7.54 (m, 4H), 7.02 (t, J=5.9 Hz, 1H), 5.11 (dd, J=5.1, 13.3 Hz, 1H), 4.49-4.25 (m, 4H), 2.98-2.82 (m, 1H), 2.64-2.55 (m, 1H), 2.43-2.31 (m, 1H), 2.05-1.92 (m, 1H). MS (ESI) m/z 418.2 [M+H]⁺

Compound 75: General procedure with variant ii) was used for the preparation from compound VI-A employing 1-(3-chloro-5-isocyanato-2-methylphenyl)-N,N-dimethylmethanamine. ¹H NMR (400 MHz, DMSO-d₆) δ=10.97 (br s, 1H), 8.82 (s, 1H), 7.72-7.50 (m, 4H), 7.13 (d, J=2.2 Hz, 1H), 6.83 (t, J=5.9 Hz, 1H), 5.12 (dd, J=5.1, 13.3 Hz, 1H), 4.52-4.25 (m, 4H), 3.30 (s, 2H), 2.98-2.84 (m, 1H), 2.65-2.56 (m, 1H), 2.43-2.32 (m, 1H), 2.25 (s, 3H), 2.14 (s, 6H), 2.05-1.95 (m, 1H). MS (ESI) m/z 498.4 [M+H]⁺

### Preparation of 1-(3-chloro-5-isocyanato-2-methylphenyl)-N,N-dimethylmethanamine

Step 1: 3-chloro-2-methyl-5-nitrobenzoic acid. To a solution of 3-chloro-2-methylbenzoic acid (10.0 g, 58.6 mmol, 1.00 eq) in sulfuric (50.0 mL) was added nitric acid (4.19 g, 64.5 mmol, 2.99 mL, 1.10 eq) dropwise at −10° C. Then the mixture was stirred at −10° C. for 1 h. The reaction mixture was poured into ice water (about 200 ml) and stirred, the precipitated solid was collected by filtration and washed with water. 3-chloro-2-methyl-5-nitrobenzoic acid.

Step 2: (3-chloro-2-methyl-5-nitrophenyl)methanol. To a solution of 3-chloro-2-methyl-5-nitrobenzoic acid (14.0 g, 64.9 mmol, 1.00 eq) in tetrahydrofuran (100 mL) was added borane dimethyl sulfide complex (10.0 M, 13.0 mL, 2.00 eq) at 0° C. Then the mixture was stirred at 25° C. for 10 h. The reaction mixture was quenched by addition of methanol (15.0 mL) at 0° C., and then filtered and concentrated under reduced pressure to give a residue. The residue was purified by column chromatography (petroleum ether/ethyl acetate=5/1) to afford (3-chloro-2-methyl-5-nitrophenyl)methanol.

Step 3: 1-chloro-3-(chloromethyl)-2-methyl-5-nitrobenzene. To a solution of (3-chloro-2-methyl-5-nitrophenyl)methanol (2.00 g, 9.92 mmol, 1.00 eq) in dichloromethane (20.0 mL) was added thionyl chloride (5.90 g, 49.6 mmol, 3.60 mL, 5.00 eq) at 0° C. Then the mixture was stirred at 25° C. for 2 h. The reaction mixture was concentrated under reduced pressure to afford 1-chloro-3-(chloromethyl)-2-methyl-5-nitrobenzene.

Step 4: 1-(3-chloro-2-methyl-5-nitrophenyl) —N,N-dimethylmethanamine. To a solution of 1-chloro-3-(chloromethyl)-2-methyl-5-nitrobenzene (2.18 g, 9.92 mmol, 1.00 eq) and triethylamine (2.51 g, 24.8 mmol, 3.45 mL, 2.50 eq) in acetonitrile (20.0 mL) was added dimethylamine hydrochloride (1.01 g, 12.4 mmol, 1.25 eq). Then the mixture was stirred at 25° C. for 10 h. The reaction mixture was concentrated under reduced pressure to give a residue. The residue was purified by column chromatography (petroleum ether/ethyl acetate=3/1) to afford 1-(3-chloro-2-methyl-5-nitrophenyl)-N,N-dimethylmethanamine.

Step 5: 3-chloro-5-((dimethylamino)methyl)-4-methylaniline. A mixture of 1-(3-chloro-2-methyl-5-nitrophenyl)-N,N-dimethylmethanamine (0.450 g, 1.97 mmol, 1.00 eq), ammonium chloride (105 mg, 1.97 mmol, 1.00 eq) and ferrous powder (549 mg, 9.84 mmol, 5.00 eq) in ethyl alcohol (6.00 mL) and water (3.00 mL) was stirred at 90° C. for 10 h. The reaction mixture was filtered, and then the filtrate was extracted with ethyl acetate (3×25.0 mL). The combined organic layers were washed with brine (10.0 mL), dried over sodium sulfate, filtered and concentrated under reduced pressure to give 3-chloro-5-((dimethylamino)methyl)-4-methylaniline.

Step 6: 1-(3-chloro-5-isocyanato-2-methylphenyl)-N,N-dimethylmethanamine. A mixture of 3-chloro-5-((dimethylamino)methyl)-4-methylaniline (0.350 g, 1.76 mmol, 1.00 eq) and triphosgene (261 mg, 881 μmol, 0.50 eq) in toluene (5.00 mL) was stirred at 110° C. for 2 h. The reaction mixture was concentrated to give 1-(3-chloro-5-isocyanato-2-methylphenyl)-N,N-dimethylmethanamine.

Compound 76: General procedure with variant ii) was used for the preparation from compound VI-A employing 1-chloro-5-isocyanato-4-methoxy-2-methylbenzene. ¹H NMR (400 MHz, DMSO-d₆) δ=10.97 (s, 1H), 8.15 (s, 1H), 8.12 (s, 1H), 7.64 (s, 1H), 7.59-7.51 (m, 2H), 7.43 (t, J=5.9 Hz, 1H), 6.95 (s, 1H), 5.11 (dd, J=5.1, 13.3 Hz, 1H), 4.48-4.22 (m, 4H), 3.83 (s, 3H), 2.96-2.82 (m, 1H), 2.62-2.54 (m, 1H), 2.38 (br dd, J=4.5, 12.9 Hz, 1H), 2.24 (s, 3H), 2.05-1.91 (m, 1H). MS (ESI) m/z 471.2 [M+H]⁺

### Preparation of 1-chloro-5-isocyanato-4-methoxy-2-methylbenzene

Step 1: 1-chloro-4-methoxy-2-methyl-5-nitrobenzene. To a solution of 4-chloro-5-methyl-2-nitrophenol (4.60 g, 24.5 mmol, 1.00 eq) in acetonitrile (50.0 mL) was added dimethyl sulfate (3.71 g, 29.4 mmol, 2.79 mL, 1.20 eq) and potassium carbonate (6.78 g, 49.1 mmol, 2.00 eq). The reaction was stirred at 80° C. for 12 h. The reaction mixture was quenched with water (50.0 mL), extracted with ethyl acetate (3×100 mL). The combined extracts were washed with water (2×50.0 mL), dried over sodium sulfate, filtered and concentrated in vacuo to give 1-chloro-4-methoxy-2-methyl-5-nitrobenzene.

Step 2: 5-chloro-2-methoxy-4-methylaniline. To a solution of 1-chloro-4-methoxy-2-methyl-5-nitrobenzene (5.00 g, 24.8 mmol, 1.00 eq) in ethanol (30.0 mL) and water (10.0 mL) was added iron powder (4.15 g, 74.4 mmol, 3.00 eq) and ammonium chloride (6.63 g, 124 mmol, 5.00 eq). The reaction was stirred at 80° C. for 12 h. The reaction mixture was filtered and concentrated in vacuo. The residue was suspended in water (50.0 mL) and extracted with ethyl acetate (3×100 mL). The combined extracts washed with water (2×50.0 mL), dried over sodium sulfate, filtered and concentrated in vacuo to give 5-chloro-2-methoxy-4-methylaniline.

Step 3: 1-chloro-5-isocyanato-4-methoxy-2-methylbenzene. To a solution of 5-chloro-2-methoxy-4-methylaniline (1.00 g, 5.83 mmol, 1.00 eq) in toluene (10.0 mL) was added triphosgene (1.73 g, 5.83 mmol, 1.00 eq). The reaction was stirred at 100° C. for 2 h. The reaction mixture was concentrated in vacuo to give 1-chloro-5-isocyanato-4-methoxy-2-methylbenzene.

Compound 77: General procedure with variant ii) was used for the preparation from compound VI-A employing 1-isocyanato-4-(trifluoromethoxy)benzene. ¹H NMR (400 MHz, DMSO-d₆) δ=10.97 (br s, 1H), 8.90 (s, 1H), 7.66 (s, 1H), 7.56 (d, J=0.8 Hz, 2H), 7.53-7.48 (m, 2H), 7.22 (d, J=8.4 Hz, 2H), 6.87 (br t, J=5.6 Hz, 1H), 5.11 (dd, J=5.2, 13.6 Hz, 1H), 4.47-4.38 (m, 3H), 4.34-4.27 (m, 1H), 2.99-2.85 (m, 1H), 2.64-2.55 (m, 1H), 2.43-2.35 (m, 1H), 2.04-1.96 (m, 1H). MS (ESI) m/z 477.2 [M+H]⁺

Compound 78: General procedure with variant ii) was used for the preparation from compound VI-A employing 1,4-dichloro-2-isocyanato-5-methylbenzene. ¹H NMR (400 MHz, DMSO-d₆) δ=10.99 (s, 1H), 8.28 (s, 1H), 8.23 (s, 1H), 7.68 (s, 1H), 7.64-7.56 (m, 3H), 7.45 (s, 1H), 5.12 (dd, J=5.1, 13.3 Hz, 1H), 4.52-4.39 (m, 3H), 4.38-4.26 (m, 1H), 3.00-2.81 (m, 1H), 2.64-2.58 (m, 1H), 2.39 (br dd, J=7.9, 12.2 Hz, 1H), 2.25 (s, 3H), 2.05-1.95 (m, 1H). MS (ESI) m/z 475.1 [M+H]⁺

Preparation of 1,4-dichloro-2-isocyanato-5-methylbenzene: To a solution of 2,5-dichloro-4-methyl-benzoic acid (1.00 g, 4.88 mmol, 1.00 eq) and triethylamine (523 mg, 5.17 mmol, 720 uL, 1.06 eq) in toluene (50.0 mL) was added diphenyl phosphorazidate (1.37 g, 4.97 mmol, 1.08 mL, 1.02 eq) at 20° C. The reaction mixture was stirred 120° C. for 2 h. The reaction mixture was concentrated and the obtained residue was suspended in dichloromethane (4.00 mL) to give 1,4-dichloro-2-isocyanato-5-methylbenzene.

Compound 79: General procedure A with variant iv) was used for the preparation from compound VI-A employing 4-isocyanato-N,N-dimethylaniline. ¹H NMR (400 MHz, DMSO-d₆+D₂O) δ=7.72-7.46 (m, 7H), 5.06 (dd, J=5.1, 13.3 Hz, 1H), 4.47-4.25 (m, 4H), 3.10 (s, 6H), 2.93-2.81 (m, 1H), 2.65-2.55 (m, 1H), 2.37 (dq, J=4.3, 13.2 Hz, 1H), 2.05-1.93 (m, 1H). MS (ESI) m/z 436.2 [M+H]⁺

Preparation of 4-isocyanato-N,N-dimethylaniline: To a solution of 4-(dimethylamino)benzoic acid (1.00 g, 6.05 mmol, 1.00 eq) in toluene (50.0 mL) was added triethylamine (0.89 mL, 6.42 mmol, 1.06 eq) and diphenylphosphoryl azide (1.34 mL, 6.17 mmol, 1.02 eq). The reaction was stirred at 20° C. for 0.5 h, then at 120° C. for 2 h. The mixture was concentrated to give a residue. Dichloromethane (4.00 mL) was added to the residue to afford the 4-isocyanato-N,N-dimethylaniline.

### Compound 80.

Step 1: To a solution of 3-((tert-butoxycarbonyl)amino)benzoic acid (500 mg, 2.11 mmol, 1.00 eq) in toluene (25.0 mL) was added triethylamine (0.31 mL, 2.23 mmol, 1.06 eq) and diphenyl phosphorazidate (0.47 mL, 2.15 mmol, 1.02 eq). The reaction was stirred at 20° C. for 0.5 h, then at 120° C. for 2 h. The mixture was concentrated under reduced pressure to afford tert-butyl-(3-isocyanatophenyl)carbamate.

Step 2: To a solution of 3-(6-(aminomethyl)-1-oxoisoindolin-2-yl)piperidine-2,6-dione hydrochloride VI-A (1.00 eq, hydrochloride) in dimethylformamide (2.00 mL) was added triethylamine (0.11 mL, 0.77 mmol, 1.20 eq) and tert-butyl (3-isocyanatophenyl)carbamate (182 mg, 0.77 mmol, 1.20 eq) at 0° C. The reaction was stirred at 20° C. for 1 h. The mixture was concentrated under reduced pressure to give a residue, which was purified by reversed phase column chromatography and lyophilized to afford tert-butyl (3-(3-((2-(2,6-dioxopiperidin-3-yl)-3-oxoisoindolin-5-yl)methyl)ureido)phenyl)carbamate.

Step 3: To a solution of tert-butyl (3-(3-((2-(2,6-dioxopiperidin-3-yl)-3-oxoisoindolin-5-yl)methyl)ureido) phenyl)carbamate (150 mg, 0.30 mmol, 1.00 eq) in methanol (1.00 mL) was added 4N of hydrochloric acid in methanol (1.00 mL). The reaction was stirred at 20° C. for 0.5 h. The mixture was concentrated and the obtained residue was purified by a standard method to afford Compound 80.

¹H NMR (400 MHz, DMSO-d₆) δ=7.83-7.71 (m, 1H), 7.68-7.61 (m, 1H), 7.56 (d, J=0.9 Hz, 2H), 7.39-7.29 (m, 1H), 7.28-7.19 (m, 1H), 7.00-6.76 (m, 1H), 5.07 (dd, J=5.1, 13.3 Hz, 1H), 4.45-4.37 (m, 3H), 4.34-4.20 (m, 1H), 2.93-2.80 (m, 1H), 2.59 (td, J=2.0, 15.3 Hz, 1H), 2.40-2.40 (m, 1H), 2.04-1.94 (m, 1H). MS (ESI) m/z 408.1 [M+H]⁺

### Compound 81:

Step 1: To a solution of 3-(6-(aminomethyl)-1-oxoisoindolin-2-yl)piperidine-2,6-dione hydrochloride VI-A (1.00 eq, hydrochloride) in dimethylformamide (2.00 mL) was added triethylamine (0.09 ml, 645 μmol, 1.00 eq) and 1-isocyanato-4-nitro-benzene (106 mg, 0.65 mmol, 1.00 eq) at 0° C. The reaction was stirred 20° C. for 1 h. The mixture was quenched with water (10.0 mL) and extracted with ethyl acetate (2×50 mL). The combined organic layers were washed with brine (30.0 mL), dried over anhydrous sodium sulfate, filtered, and the filtrate was concentrated under reduced pressure to afford 1-[[2-(2,6-dioxo-3-piperidyl)-3-oxo-isoindolin-5-yl]methyl]-3-(4-nitrophenyl)urea.

Step 2: To a solution of 1-[[2-(2,6-dioxo-3-piperidyl)-3-oxo-isoindolin-5-yl]methyl]-3-(4-nitrophenyl)urea (150 mg, 343 μmol, 1.00 eq) in methanol (3.00 mL) was added Pd/C 10.0% weight on C (5 mg,) and hydrochloric acid 4M (8.57 μL, 0.10 eq). The reaction was stirred at 20° C. for 2 h under hydrogen atmosphere (15 psi). The mixture was filtered, and the filtrate was concentrated and the obtained residue was purified by standard methods to afford Compound 81.

¹H NMR (400 MHz, DMSO-d₆) δ=11.0 (s, 1H), 10.52-9.67 (b, 3H), 9.24 (s, 1H), 7.66 (s, 1H), 7.57 (s, 2H), 7.53 (d, J=8.9 Hz, 2H), 7.25 (d, J=8.9 Hz, 2H), 7.08 (br s, 1H), 5.11 (dd, J=5.0, 13.2 Hz, 1H), 4.48-4.27 (m, 4H), 2.98-2.81 (m, 1H), 2.60 (br d, J=17.4 Hz, 1H), 2.45-2.36 (m, 1H), 2.05-1.95 (m, 1H). MS (ESI) m/z 408.4 [M+H]⁺

### Compound 82:

Step 1: To a solution of (3-chloro-2-methyl-5-nitrophenyl)methanol (2.00 g, 9.92 mmol, 1.00 eq) in dichloromethane (20.0 mL) was added thionyl chloride (3.60 mL, 49.6 mmol, 5.00 eq). The reaction was stirred at 25° C. for 2 h. The mixture was concentrated under reduced pressure to afford 1-chloro-3-(chloromethyl)-2-methyl-5-nitro-benzene.

Step 2: To a solution of 1-chloro-3-(chloromethyl)-2-methyl-5-nitrobenzene (2.20 g, 10.0 mmol, 1.00 eq) and triethylamine (3.48 mL, 25.0 mmol, 2.50 eq) in acetonitrile (20.0 mL) was added morpholine (1.14 mL, 13.0 mmol, 1.30 eq). The reaction was stirred at 25° C. for 10 h. The mixture was concentrated under reduced pressure to give a residue, which was purified by silica gel column chromatography (petroleum ether/ethyl acetate=3/1) to afford 4-(3-chloro-2-methyl-5-nitrobenzyl) morpholine.

Step 3: To a mixture of ethanol (8.00 mL) and water (4.00 mL) was added 4-(3-chloro-2-methyl-5-nitrobenzyl)morpholine (0.500 g, 1.85 mmol, 1.00 eq), ammonium chloride (98.8 mg, 1.85 mmol, 1.00 eq) and ferrous powder (516 mg, 9.23 mmol, 5.00 eq). The reaction was stirred at 90° C. for 10 h. The mixture was filtered and washed with ethyl acetate (20.0 mL). The filtrate was extracted with ethyl acetate (3×25.0 mL), and the combined organic layers were washed with brine (20.0 mL), dried over sodium sulfate, filtered, and concentrated under reduced pressure to afford 3-chloro-4-methyl-5-(morpholinomethyl)aniline.

Step 4: To a solution of 3-chloro-4-methyl-5-(morpholinomethyl)aniline (0.100 g, 0.41 mol, 1.00 eq) in tetrahydrofuran (1.00 mL) was added 1,1′-carbonyldiimidazole (74.1 mg, 0.46 mmol, 1.10 eq). The mixture was stirred at 25° C. for 2 h, then 3-(6-(aminomethyl)-1-oxoisoindolin-2-yl)piperidine-2,6-dione hydrochloride VI-A (129 mg, 0.41 mmol, 1.00 eq, hydrochloride) was added. The reaction was stirred at 25° C. for 10 h. The mixture was adjusted to pH=5 by addition of HCl (1N), then it was concentrated and the obtained residue was purified by standard methods to afford Compound.

¹H NMR (400 MHz, DMSO-d₆) δ=11.0 (s, 1H), 8.82 (s, 1H), 8.21 (s, 1H), 7.68-7.61 (m, 2H), 7.56 (d, J=0.6 Hz, 2H), 7.12 (d, J=2.0 Hz, 1H), 6.83 (br t, J=5.6 Hz, 1H), 5.12 (dd, J=5.0, 13.1 Hz, 1H), 4.46-4.27 (m, 4H), 3.54 (br s, 4H), 3.38 (br s, 2H), 2.97-2.85 (m, 1H), 2.63-2.59 (m, 1H), 2.42-2.38 (m, 1H), 2.35 (br s, 4H), 2.26 (s, 3H), 2.04-1.94 (m, 1H). MS (ESI) m/z 540.1 [M+H]⁺

Compound 83: General procedure A with variant iv) was used for the preparation from compound VI-A employing 4-isocyanato-1-methyl-1H-pyrazole. ¹H NMR (400 MHz, DMSO-d₆) δ=10.98 (br s, 1H), 8.85 (s, 1H), 7.64 (s, 1H), 7.58-7.52 (m, 2H), 7.47 (d, J=2.4 Hz, 1H), 7.28 (br s, 1H), 6.05 (d, J=2.0 Hz, 1H), 5.11 (dd, J=4.8, 13.2 Hz, 1H), 4.49-4.26 (m, 4H), 3.68 (s, 3H), 2.98-2.85 (m, 1H), 2.64-2.55 (m, 1H), 2.46-2.36 (m, 1H), 2.06-1.94 (m, 1H). MS (ESI) m/z 397.1 [M+H]⁺

Preparation of 4-isocyanato-1-methyl-1H-pyrazole: To a mixture of 1-methyl-1H-pyrazole-4-carboxylic acid (400 mg, 3.17 mmol, 1.00 eq) and triethylamine (0.55 mL, 3.96 mmol, 1.25 eq) in toluene (5.00 mL) was added diphenylphosphoryl azide (0.83 mL, 3.81 mmol, 1.20 eq) in one portion at 20° C. The mixture was stirred at 105° C. under nitrogen for 3 h. The mixture was cooled to 20° C., then concentrated under reduced pressure to afford 4-isocyanato-1-methyl-1H-pyrazole.

Compound 84: General procedure A with variant iv) was used for the preparation from compound VI-A employing 3-isocyanatothiophene. ¹H NMR (400 MHz, DMSO-d₆) δ=11.04 (s, 1H), 8.99 (s, 1H), 7.71 (s, 1H), 7.61 (s, 2H), 7.42 (dd, J=3.2, 5.2 Hz, 1H), 7.22 (dd, J=1.2, 3.2 Hz, 1H), 7.04 (dd, J=1.2, 5.2 Hz, 1H), 6.79 (t, J=6.0 Hz, 1H), 5.17 (dd, J=5.0, 13.2 Hz, 1H), 4.51-4.33 (m, 4H), 3.02-2.90 (m, 1H), 2.75-2.60 (m, 1H), 2.51-2.37 (m, 1H), 2.10-2.00 (m, 1H). MS (ESI) m/z 399.0 [M+H]⁺

Preparation of 3-isocyanatothiophene: To a suspension of thiophene-3-carboxylic acid (300 mg, 2.34 mmol, 1.00 eq) and triethylamine (407 μL, 2.93 mmol, 1.25 eq) in dry toluene (5.00 mL) was added diphenylphosphoryl azide (609 μL, 2.81 mmol, 1.20 eq) at 25° C. under nitrogen. The reaction was stirred at 25° C. for 30 min, then heated to 100° C. for 2 h. The mixture was concentrated under reduced pressure to afford 3-isocyanatothiophene.

Compound 85: General procedure A with variant iv) was used for the preparation from compound VI-A employing 1-chloro-5-isocyanato-2,4-dimethylbenzene. ¹H NMR (400 MHz, DMSO-d₆) δ=10.98 (br s, 1H), 7.98 (s, 1H), 7.84 (br d, J=3.1 Hz, 1H), 7.67 (s, 1H), 7.58 (d, J=0.9 Hz, 2H), 7.25-7.15 (m, 1H), 7.09 (s, 1H), 5.12 (dd, J=5.0, 13.3 Hz, 1H), 4.50-4.24 (m, 4H), 3.00-2.81 (m, 1H), 2.62-2.55 (m, 1H), 2.43-2.36 (m, 1H), 2.21 (s, 3H), 2.14 (s, 3H), 2.01 (ddd, J=2.4, 5.3, 10.0 Hz, 1H). MS (ESI) m/z 455.2 [M+H]⁺

### Preparation of 1-chloro-5-isocyanato-2,4-dimethylbenzene

Step 1: To a solution of 2,4-dimethyl-5-nitroaniline (500 mg, 3.01 mmol, 1.00 eq) in concentrated hydrochloric acid (9.00 mL) was added a solution of sodium nitrite (208 mg, 3.01 mmol, 1.00 eq) in water (0.60 mL) at 0° C. The reaction was stirred at 0° C. for 1 h, then cuprous chloride (477 mg, 4.81 mmol, 1.60 eq) was added at 0° C. The reaction was stirred at 20° C. for 11 h. Water (50.0 mL) was added, followed by potassium carbonate until pH=7: The mixture was extracted with ethyl acetate (3×100 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=1/0 to 10/1) to afford 1-chloro-2,4-dimethyl-5-nitrobenzene.

Step 2: To a solution of 1-chloro-2,4-dimethyl-5-nitrobenzene (350 mg, 1.89 mmol, 1.00 eq) in ethanol (12.0 mL) and water (3.00 mL) was added iron powder (315 mg, 5.66 mmol, 3 eq) and ammonium chloride (504 mg, 9.43 mmol, 5.00 eq). The reaction was stirred at 80° C. for 2 h. The mixture was filtered and concentrated under reduced pressure to give a residue. The residue was purified by silica gel column chromatography (petroleum ether/ethyl acetate=1/0 to 10/1) to afford 5-chloro-2,4-dimethylaniline.

Step 3: To a solution of 5-chloro-2,4-dimethylaniline (270 mg, 1.73 mmol, 1.00 eq) in toluene (10.0 mL) was added triphosgene (515 mg, 1.73 mmol, 1.00 eq). The reaction was stirred at 100° C. for 2 h. The mixture was concentrated to afford 1-chloro-5-isocyanato-2,4-dimethylbenzene.

Compound 86: General procedure A with variant iv) was used for the preparation from compound VI-A employing 2-chloro-4-isocyanato-3-methoxy-1-methylbenzene. ¹H NMR (400 MHz, DMSO-d₆) δ=10.9 (br s, 1H), 8.24 (s, 1H), 8.02 (d, J=8.4 Hz, 1H), 7.68 (s, 1H), 7.57 (d, J=0.7 Hz, 2H), 7.44 (br t, J=5.8 Hz, 1H), 6.99 (d, J=8.7 Hz, 1H), 5.12 (dd, J=5.1, 13.3 Hz, 1H), 4.48-4.37 (m, 3H), 4.36-4.25 (m, 1H), 3.74 (s, 3H), 2.96-2.83 (m, 1H), 2.65-2.55 (m, 1H), 2.42-2.34 (m, 1H), 2.25 (s, 3H), 2.05-1.91 (m, 1H). MS (ESI) m/z 471.2 [M+H]⁺

Preparation of 2-chloro-4-isocyanato-3-methoxy-1-methylbenzene: To a solution of 3-chloro-2-methoxy-4-methylbenzoic acid (300 mg, 1.50 mmol, 1.00 eq) in toluene (15.0 mL) was added triethylamine (0.22 mL, 1.59 mmol, 1.06 eq) and diphenyl phosphorazidate (0.33 mL, 1.53 mmol, 1.02 eq). The reaction was stirred at 20° C. for 0.5 h, then at 120° C. for 2 h. The mixture was concentrated under reduced pressure to give a residue. Dichloromethane (4.00 mL) was added to afford 2-chloro-4-isocyanato-3-methoxy-1-methylbenzene.

Compound 87: General procedure A with variant iv) was used for the preparation from compound VI-A employing 2,3-dichloro-1-isocyanato-4-methylbenzene. ¹H NMR (400 MHz, DMSO-d₆) δ=10.9 (br s, 1H), 8.24 (s, 1H), 8.07-7.97 (m, 1H), 7.68 (s, 1H), 7.63-7.50 (m, 3H), 7.24 (d, J=8.7 Hz, 1H), 5.12 (dd, J=5.1, 13.3 Hz, 1H), 4.49-4.38 (m, 3H), 4.37-4.27 (m, 1H), 2.98-2.85 (m, 1H), 2.65-2.56 (m, 1H), 2.40 (br dd, J=4.5, 13.1 Hz, 1H), 2.32 (s, 3H), 2.05-1.97 (m, 1H). MS (ESI) m/z 475.2 [M+H]⁺

Preparation of 2,3-dichloro-1-isocyanato-4-methylbenzene: To a solution of 3-chloro-4-methylaniline (5.00 g, 35.3 mmol, 1.00 eq) in N,N-dimethyformamide (50.0 mL) at 0° C. was added 1-chloropyrrolidine-2, 5-dione (5.00 g, 37.4 mmol, 1.06 eq) in dimethyformamide (20.0 mL) dropwise. The reaction was stirred at 20° C. for 12 h. Water (50.0 mL) was added and the mixture was extracted with ethyl acetate (2×100 mL). The combined organic layers were washed with brine (2×50.0 mL), dried over sodium sulfate, and concentrated under reduced pressure to give a residue. The residue was purified by silica gel column chromatography (petroleum ether/ethyl acetate=1/0 to 3/1) to give 2,3-dichloro-4-methylaniline.

Compound 88: General procedure A with variant iii) was used for the preparation from compound VI-A employing phenyl (1-methyl-1H-pyrazol-3-yl)carbamate. ¹H NMR (400 MHz, DMSO-d₆) δ=10.98 (br s, 1H), 8.85 (s, 1H), 7.64 (s, 1H), 7.58-7.52 (m, 2H), 7.47 (d, J=2.4 Hz, 1H), 7.28 (br s, 1H), 6.05 (d, J=2.0 Hz, 1H), 5.11 (dd, J=4.8, 13.2 Hz, 1H), 4.49-4.26 (m, 4H), 3.68 (s, 3H), 2.98-2.85 (m, 1H), 2.64-2.55 (m, 1H), 2.46-2.36 (m, 1H), 2.06-1.94 (m, 1H). MS (ESI) m/z 397.1 [M+H]⁺

### Preparation of phenyl (1-methyl-1H-pyrazol-3-yl)carbamate

Step 1: To a solution of 1-methyl-1H-pyrazole-3-carboxylic acid (500 mg, 3.96 mmol, 1.00 eq), triethylamine (0.55 mL, 3.96 mmol, 1.00 eq) in toluene (5.00 mL) was added diphenyl phosphorazidate (0.86 mL, 3.96 mmol, 1.00 eq) at 25° C. The reaction was stirred at 25° C. for 30 min. The mixture was concentrated under reduced pressure to afford 1-methyl-1H-pyrazole-3-carbonyl azide.

Step 2: To a solution of phenol (2.96 mL, 33.7 mmol, 11.3 eq) in toluene (22.0 mL) was added 1-methyl-1H-pyrazole-3-carbonyl azide (450 mg, 2.98 mmol, 1.00 eq) at 100° C. The reaction was stirred at 100° C. for 3 hr. The mixture was concentrated under reduced pressure to give a residue. The residue was purified by silica gel column chromatography (petroleum ether/ethyl acetate=0/1 to 3/1) to give phenyl (1-methyl-1H-pyrazol-3-yl)carbamate.

Compound 89: General procedure A with variant iv) was used for the preparation from compound VI-A employing 3-isocyanato-N,N-dimethylaniline. ¹H NMR (400 MHz, DMSO-d₆) δ=11.00 (br s, 1H), 8.52 (s, 1H), 8.32 (s, 1H), 7.66 (s, 1H), 7.56 (d, J=0.9 Hz, 2H), 7.04-6.97 (m, 1H), 6.88 (t, J=2.1 Hz, 1H), 6.79-6.65 (m, 2H), 6.30 (dd, J=2.1, 8.2 Hz, 1H), 5.12 (dd, J=5.1, 13.3 Hz, 1H), 4.46-4.26 (m, 4H), 3.02-2.89 (m, 1H), 2.85 (s, 6H), 2.60 (td, J=1.9, 15.4 Hz, 1H), 2.39 (br dd, J=4.5, 13.0 Hz, 1H), 2.05-1.95 (m, 1H). MS (ESI) m/z 436.2 [M+H]⁺

Preparation of 3-isocyanato-N,N-dimethylaniline: To a solution of 3-(dimethylamino)benzoic acid (1.00 g, 6.05 mmol, 1.00 eq) in toluene (50.0 mL) was added triethylamine (0.89 mL, 6.42 mmol, 1.06 eq) and diphenylphosphoryl azide (1.34 mL, 6.17 mmol, 1.02 eq). The reaction was stirred at 20° C. for 0.5 h, then at 120° C. for 2 h. The mixture was concentrated under reduced pressure to give a residue. Dichloromethane (4.00 mL) was added to the residue to afford 4-isocyanato-N,N-dimethylaniline.

Compound 90: General procedure A with variant iv) was used for the preparation from compound VI-A employing 1-isocyanato-3-(trifluoromethoxy)benzene. ¹H NMR (400 MHz, DMSO-d₆) δ=10.98 (br s, 1H), 9.02 (s, 1H), 7.67 (s, 2H), 7.57 (d, J=0.7 Hz, 2H), 7.37-7.30 (m, 1H), 7.29-7.22 (m, 1H), 6.91 (br t, J=5.9 Hz, 1H), 6.88-6.83 (m, 1H), 5.12 (dd, J=5.1, 13.3 Hz, 1H), 4.49-4.36 (m, 3H), 4.36-4.26 (m, 1H), 2.98-2.85 (m, 1H), 2.60 (td, J=2.0, 15.4 Hz, 1H), 2.40 (br dd, J=4.5, 13.0 Hz, 1H), 2.05-1.96 (m, 1H). MS (ESI) m/z 477.2 [M+H]⁺

Compound 91: General procedure A with variant iv) was used for the preparation from compound VI-A employing 2-isocyanatothiophene. ¹H NMR (400 MHz, DMSO-d₆) δ=10.96 (br s, 1H), 9.64 (s, 1H), 7.65 (s, 1H), 7.56 (s, 2H), 6.86 (br t, J=6.0 Hz, 1H), 6.79-6.72 (m, 2H), 6.44 (dd, J=1.6, 3.2 Hz, 1H), 5.11 (dd, J=5.2, 13.2 Hz, 1H), 4.49-4.24 (m, 4H), 2.97-2.84 (m, 1H), 2.64-2.55 (m, 1H), 2.44-2.36 (m, 1H), 2.04-1.95 (m, 1H). MS (ESI) m/z 399.0 [M+H]⁺

### Preparation of 2-isocyanatothiophene

Step 1: To a solution of thiophene-2-carboxylic acid (300 mg, 2.34 mmol, 1.00 eq), triethylamine (0.33 mL, 2.34 mmol, 1.00 eq) in toluene (1.50 mL) was added diphenyl phosphorazidate (0.51 mL, 2.34 mmol, 1.00 eq) at 25° C. The reaction was stirred at 25° C. for 30 min. Water (1.00 mL) was added, and the mixture was extracted with ethyl acetate (3×5.00 ml). The organic layers were combined, washed with saturated sodium bicarbonate (1.00 ml), dried over magnesium sulfate, filtered, and concentrated under reduced pressure to give thiophene-2-carbonyl azide.

Step 2: Thiophene-2-carbonyl azide (400 mg, 2.61 mmol, 1.00 eq) in toluene (4.00 mL) was stirred at 120° C. for 30 min to afford 2-isocyanatothiophene.

Compound 92: General procedure A with variant iii) was used for the preparation from compound VI-A employing phenyl thiazol-2-ylcarbamate. ¹H NMR (400 MHz, DMSO-d₆) δ=11.0 (br s, 1H), 10.8-10.5 (m, 1H), 8.38 (s, 1H), 7.65 (s, 1H), 7.57 (d, J=0.6 Hz, 2H), 7.31 (d, J=3.6 Hz, 1H), 7.27 (br s, 1H), 7.02 (d, J=3.6 Hz, 1H), 5.11 (dd, J=5.1, 13.3 Hz, 1H), 4.51-4.37 (m, 3H), 4.34-4.25 (m, 1H), 3.02-2.84 (m, 1H), 2.69-2.55 (m, 1H), 2.46-2.28 (m, 1H), 2.06-1.91 (m, 1H). MS (ESI) m/z 400.2 [M+H]⁺

Preparation of phenyl thiazol-2-ylcarbamate: To a solution of thiazol-2-amine (1.00 g, 9.99 mmol, 1.00 eq) in dichloromethane (5.00 mL) was added pyridine (4.84 mL, 60.0 mmol, 6.00 eq) and phenyl chloroformate (1.50 mL, 12.0 mmol, 1.20 eq). The reaction was stirred at 20° C. for 12 h. The mixture was concentrated under reduced pressure to give a residue. The residue was purified by silica gel column chromatography (petroleum ether/ethyl acetate=1/0 to 1/1) to give phenyl thiazol-2-ylcarbamate.

Compound 93: General procedure A with variant iv) was used for the preparation from compound VI-A employing 2-chloro-4-isocyanato-1,3-dimethylbenzene. ¹H NMR (400 MHz, DMSO-d₆) δ=11.0 (s, 1H), 7.97 (s, 1H), 7.67 (s, 1H), 7.57 (d, J=0.7 Hz, 2H), 7.54 (d, J=8.3 Hz, 1H), 7.09 (d, J=8.3 Hz, 1H), 7.03 (br t, J=5.9 Hz, 1H), 5.12 (dd, J=5.0, 13.3 Hz, 1H), 4.50-4.36 (m, 3H), 4.35-4.26 (m, 1H), 2.98-2.83 (m, 1H), 2.60 (br d, J=18.0 Hz, 1H), 2.40 (br dd, J=4.4, 13.1 Hz, 1H), 2.27 (s, 3H), 2.24 (s, 3H), 2.05-1.95 (m, 1H). MS (ESI) m/z 455.2 [M+H]⁺

### Preparation of 2-chloro-4-isocyanato-1,3-dimethylbenzene

Step 1: Nitric acid (1.50 mL, 33.4 mmol, 1.00 eq) was added dropwise to a solution of 2-chloro-1,3-dimethylbenzene (4.43 mL, 33.4 mmol, 1.00 eq) in sulfuric acid (20.0 mL) at 0° C. The reaction was stirred at 20° C. for 2 h. The mixture was poured into ice water (20.0 mL) and extracted with ethyl acetate (2×20.0 mL). The combined organic layers were washed with brine (30.0 mL), dried over anhydrous sodium sulfate, filtered, and the filtrate was concentrated under reduced pressure to give a residue. The residue was purified by silica gel column chromatography (petroleum ether/ethyl acetate=1/0) to afford 2-chloro-1,3-dimethyl-4-nitrobenzene.

Step 2: To a solution of 2-chloro-1,3-dimethyl-4-nitrobenzene (2.10 g, 11.3 mmol, 1.00 eq) in ethanol (24.0 mL) and water (8.00 mL) was added ammonium chloride (6.05 g, 113 mmol, 10.0 eq) and Fe (3.79 g, 67.9 mmol, 6.00 eq). The reaction was stirred at 80° C. for 2 h. The mixture was filtered, and the filtrate was concentrated under reduced pressure to give a residue. The residue was purified by silica gel column chromatography (petroleum ether/ethyl acetate=1/0 to 3/1) to afford 3-chloro-2,4-dimethylaniline.

Step 3: To a solution of 3-chloro-2,4-dimethylaniline (300 mg, 1.93 mmol, 1.00 eq) in toluene (12.0 mL) was added bis(trichloromethyl) carbonate (572 mg, 1.93 mmol, 1.00 eq). The reaction was stirred at 100° C. for 3 h. The mixture was concentrated under reduced pressure to give a residue. Dichloromethane (1.00 mL) was added to the residue to afford 2-chloro-4-isocyanato-1,3-dimethylbenzene.

Compound 94: General procedure A with variant iii) was used for the preparation from compound VI-A employing phenyl isoxazol-3-ylcarbamate. ¹H NMR (400 MHz, DMSO-d₆) δ=11.0 (s, 1H), 9.63 (s, 1H), 8.67 (d, J=1.6 Hz, 1H), 7.65 (s, 1H), 7.59-7.51 (m, 2H), 7.12 (t, J=6.0 Hz, 1H), 6.72 (d, J=1.6 Hz, 1H), 5.12 (dd, J=4.2, 13.2 Hz, 1H), 4.51-4.24 (m, 4H), 3.00-2.80 (m, 1H), 2.71-2.58 (m, 1H), 2.39-2.25 (m, 1H), 2.06-1.91 (m, 1H). LCMS m/z=384.0 [M+H]⁺

Preparation of phenyl isoxazol-3-ylcarbamate: To a solution of isoxazol-3-amine (500 mg, 5.95 mmol, 1.00 eq) in tetrahydrofuran (20.0 mL) were added phenyl chloroformate (1.02 g, 6.54 mmol, 1.10 eq) and triethylamine (1.20 g, 11.9 mmol, 2.00 eq) dropwise at 20° C. The reaction was stirred at 25° C. for 12 h. The mixture was concentrated under reduced pressure to give a residue. The residue was purified by reversed phase column chromatography to afford phenyl isoxazol-3-ylcarbamate.

Compound 95: To a mixture of nicotinic acid (500 mg, 4.06 mmol, 1.00 eq) and triethylamine (534 mg, 5.28 mmol, 1.30 eq) in toluene (5 mL) was added diphenylphosphoryl azide (1.68 g, 6.09 mmol, 1.50 eq) dropwise at 20° C. The reaction was stirred at 20° C. for 1 h. Triethylamine (1.64 g, 16.3 mmol, 4.00 eq) and 3-(6-(aminomethyl)-1-oxoisoindolin-2-yl)piperidine-2,6-dione hydrochloride VI-A (555 mg, 1.79 mmol, 0.44 eq, hydrochloride) were added in one portion. The reaction was stirred at 100° C. for 2 h. The mixture was diluted with ethyl acetate (50 mL) and poured into saturated aqueous sodium bicarbonate (50 mL). The organic layer was separated, and the aqueous layer was extracted with ethyl acetate (10×50 mL). The combined organic layers were dried over anhydrous sodium sulfate, filtered, and the filtrate was concentrated and the obtained residue was purified by standard methods to afford Compound 95. ¹H NMR (400 MHz, DMSO-d₆) δ=11.0 (s, 1H), 8.94 (s, 1H), 8.56 (d, J=2.4 Hz, 1H), 8.15-8.08 (m, 1H), 7.94-7.86 (m, 1H), 7.68 (s, 1H), 7.57 (s, 2H), 7.29-7.20 (m, 1H), 7.02 (m, 1H), 5.17-5.06 (m, 1H), 4.48-4.28 (m, 4H), 2.94-2.86 (m, 1H), 2.65-2.58 (m, 1H), 2.46-2.37 (m, 1H), 2.05-1.96 (m, 1H). LCMS m/z 394.1 [M+H]⁺

Compound 96: General procedure A with variant iv) was used for the preparation from compound VI-A employing 1,4-dichloro-2-isocyanato-3,5-dimethylbenzene. ¹H NMR (400 MHz, DMSO-d₆) δ=11.0 (br s, 1H), 8.06 (s, 1H), 7.69 (s, 1H), 7.55 (s, 2H), 7.40 (s, 1H), 6.94 (br s, 1H), 5.11 (dd, J=5.1, 13.3 Hz, 1H), 4.49-4.27 (m, 4H), 2.97-2.83 (m, 1H), 2.60 (td, J=2.0, 15.3 Hz, 1H), 2.40 (br dd, J=4.5, 13.0 Hz, 1H), 2.32 (s, 3H), 2.28-2.21 (m, 3H), 2.04-1.96 (m, 1H). MS (ESI) m/z 489.2 [M+H]⁺

### Preparation of 1,4-dichloro-2-isocyanato-3,5-dimethylbenzene

Step 1: To a solution of 3-chloro-2,4-dimethylaniline (780 mg, 5.01 mmol, 1.00 eq) in dimethylformamide (10.5 mL) was added a solution of 1-chloropyrrolidine-2,5-dione (709 mg, 5.31 mmol, 1.06 eq) in dimethylformamide (7.00 mL). The reaction was stirred at 20° C. for 12 h, then it was concentrated under reduced pressure to give a residue. The residue was purified by silica gel column chromatography (petroleum ether/ethyl acetate=10/1) to give 3,6-dichloro-2,4-dimethylaniline.

Step 2: To a solution of 3,6-dichloro-2,4-dimethylaniline (290 mg, 1.53 mmol, 1.00 eq) in toluene (12.0 mL) was added bis(trichloromethyl) carbonate (453 mg, 1.53 mmol, 1.00 eq).

The reaction was stirred at 100° C. for 3 h. The mixture was concentrated under reduced pressure to give a residue. Dichloromethane (1.00 mL) was added to the residue to afford 1,4-dichloro-2-isocyanato-3,5-dimethylbenzene.

### Compound 97:

Step 1: To a mixture of 5-methylisoxazole-3-carboxylic acid (700 mg, 5.51 mmol, 1.00 eq) and triethylamine (725 mg, 7.16 mmol, 1.30 eq) in toluene (5 mL) was added diphenylphosphoryl azide (2.58 g, 9.36 mmol, 1.70 eq) dropwise at 20° C. The mixture was stirred at 20° C. for 1 h. Triethylamine (836 mg, 8.26 mmol, 1.5 eq) and 3-(6-(aminomethyl)-1-oxo-isoindolin-2-yl)piperidine-2,6-dione hydrochloride VI-A (500 mg, 1.61 mmol, 0.293 eq, hydrochloride) were added, and the reaction was stirred at 100° C. for 2 h. The mixture was cooled to 20° C. and diluted with ethyl acetate (50.0 mL). The mixture was poured into saturated aqueous sodium bicarbonate (100 mL), the organic layer was separated, and the aqueous layer was extracted with ethyl acetate (5×50 mL). The combined organic layers were dried over anhydrous sodium sulfate, filtered, and the filtrate was concentrated and the obtained residue was purified by standard methods to afford Compound 97. ¹H NMR (400 MHz, DMSO-d₆) δ=10.9 (br s, 1H), 9.57-9.43 (m, 1H), 7.64 (s, 1H), 7.56 (s, 2H), 7.13 (br s, 1H), 6.40 (s, 1H), 5.19-5.04 (m, 1H), 4.49-4.37 (m, 3H), 4.35-4.24 (m, 1H), 2.98-2.83 (m, 1H), 2.64-2.55 (m, 1H), 2.43-2.36 (m, 1H), 2.32 (s, 3H), 2.05-1.93 (m, 1H). LCMS m/z 398.1 [M+H]⁺

Compound 98: General procedure A with variant iii) was used for the preparation from compound VI-A employing phenyl 1H-pyrrol-3-ylcarbamate. ¹H NMR (400 MHz, DMSO-d₆) δ=11.0 (br s, 1H), 10.3 (br s, 1H), 7.98 (s, 1H), 7.63 (s, 1H), 7.56-7.51 (m, 2H), 6.73 (s, 1H), 6.52 (d, J=2.4 Hz, 1H), 6.43 (t, J=6.0 Hz, 1H), 5.85 (d, J=1.6 Hz, 1H), 5.11 (dd, J=7.6, 13.2 Hz, 1H), 4.49-4.23 (m, 4H), 2.97-2.84 (m, 1H), 2.64-2.55 (m, 1H), 2.47-2.35 (m, 1H), 2.03-1.95 (m, 1H). MS (ESI) m/z 382.1 [M+H]⁺

### Preparation of phenyl 1H-pyrrol-3-ylcarbamate

Step 1: To a solution of 1H-pyrrole-3-carboxylic acid (500 mg, 4.50 mmol, 1.00 eq), triethylamine (0.63 mL, 4.50 mmol, 1.00 eq) in toluene (1.00 mL) was added diphenyl phosphorazidate (0.97 mL, 4.50 mmol, 1.00 eq) at 25° C. The reaction was stirred at 25° C. for 1 hr. The mixture was used in the next step directly.

Step 2: To a solution of phenol (4.39 mL, 49.9 mmol, 11.3 eq) in toluene (15.0 mL) was added 1H-pyrrole-3-carbonyl azide (600 mg, 4.41 mmol, 1.00 eq) at 100° C. The mixture was stirred at 100° C. for 1 hr. The mixture was concentrated under reduced pressure to give a residue. The residue was purified by silica gel column chromatography (petroleum ether/ethyl acetate=0/1 to 4/1) to give phenyl 1H-pyrrol-3-ylcarbamate.

Compound 99: General procedure A with variant iii) was used for the preparation from compound VI-A employing phenyl thiazol-4-ylcarbamate. ¹H NMR (400 MHz, DMSO-d₆) δ=11.0 (s, 1H), 9.46 (s, 1H), 8.89 (d, J=2.0 Hz, 1H), 7.66 (s, 1H), 7.57 (s, 2H), 7.21 (d, J=2.4 Hz, 1H), 6.94 (br t, J=5.6 Hz, 1H), 5.12 (dd, J=5.2, 13.2 Hz, 1H), 4.49-4.26 (m, 4H), 2.98-2.85 (m, 1H), 2.65-2.56 (m, 1H), 2.45-2.37 (m, 1H), 2.06-1.94 (m, 1H). MS (ESI) m/z 400.2 [M+H]⁺

### Preparation of phenyl thiazol-4-ylcarbamate

Step 1: To a solution of thiazole-4-carboxylic acid (0.50 g, 3.87 mmol, 1.00 eq), triethylamine (0.54 mL, 3.87 mmol, 1.00 eq) in toluene (4.00 mL) was added diphenyl phosphorazidate (0.84 mL, 1.07 g, 3.87 mmol, 1.00 eq) at 25° C. The mixture was stirred at 25° C. for 30 min. The mixture was used in the next step directly.

Step 2: To a solution of phenol (4.39 mL, 49.9 mmol, 11.3 eq) in toluene (15.0 mL) was added thiazole-4-carbonyl azide (0.60 g, 4.41 mmol, 1.00 eq) at 100° C. The reaction was stirred at 100° C. for 1 hr. The mixture was concentrated under reduced pressure to give a residue. The residue was purified by standard methods to give phenyl thiazol-4-ylcarbamate.

Compound 100: General procedure A with variant iv) was used for the preparation from compound VI-A employing 2,3-dichloro-4-isocyanato-1,5-dimethylbenzene.

¹H NMR (400 MHz, DMSO-d₆) δ=11.0 (s, 1H), 8.01 (s, 1H), 7.69 (s, 1H), 7.55 (s, 2H), 7.22 (s, 1H), 6.91 (br t, J=5.4 Hz, 1H), 5.11 (dd, J=5.1, 13.3 Hz, 1H), 4.52-4.24 (m, 4H), 2.91 (ddd, J=5.4, 13.7, 17.3 Hz, 1H), 2.63-2.57 (m, 1H), 2.44-2.38 (m, 1H), 2.34 (s, 3H), 2.17 (s, 3H), 2.05-1.93 (m, 1H). MS (ESI) m/z 489.2 [M+H]⁺

### Preparation of 2,3-dichloro-4-isocyanato-1,5-dimethylbenzene

Step 1: To a solution of 5-chloro-2,4-dimethylaniline (1.50 g, 9.64 mmol, 1.00 eq) in dimethyformamide (15.00 mL) was added a solution of 1-chloropyrrolidine-2,5-dione (1.36 g, 10.2 mmol, 1.06 eq) in dimethyformamide (6.00 mL). The reaction was stirred at 20° C. for 12 h. Water (20.0 mL) was added to the mixture and it was extracted with ethyl acetate (2×100 mL). The organic layer was washed with brine (2×50.0 mL), dried over with anhydrous sodium sulfate, filtered, and concentrated under reduced pressure. The residue was purified by silica gel column chromatography (petroleum ether/ethyl acetate=1/0 to 10/1) to give 2,3-dichloro-4,6-dimethylaniline.

Step 2: To a solution of 2,3-dichloro-4,6-dimethylaniline (500 mg, 2.63 mmol, 1.00 eq) in toluene (10.0 mL) was added triphosgene (781 mg, 2.63 mmol, 1.00 eq). The reaction was stirred at 100° C. for 2 h. The mixture was concentrated to give 2,3-dichloro-4-isocyanato-1,5-dimethylbenzene.

### Compound 101:

Step 1: To a mixture of 2-methyl-5-nitrobenzoic acid (5.00 g, 27.6 mmol, 1.00 eq) in tetrahydrofuran (138 mL) was added borane dimethyl sulfide complex (10.0 M, 5.52 mL, 2.00 eq) dropwise at 20° C. The reaction was stirred at 75° C. under nitrogen atmosphere for 4 h. The mixture was cooled to 5° C. and methanol/water (25.0 mL, v/v=1:1) was added, followed by 5N hydrochloric acid (50.0 mL). The mixture was concentrated under reduced pressure to give a slurry, which was poured into water (50.0 ml) and extracted with ethyl acetate (4×50.0 mL). The combined organic layers were washed with brine (50.0 mL), dried over sodium sulfate, filtered, and the filtrate was concentrated under reduced pressure to give a residue. The residue was purified by silica gel column chromatography (petroleum ether/ethyl acetate=100/1 to 80/20) to afford (2-methyl-5-nitrophenyl)methanol.

Step 2: To a mixture of (2-methyl-5-nitrophenyl)methanol (1.00 g, 5.98 mmol, 1.00 eq) in dichloromethane (10.0 mL) were added thionyl chloride (4.34 mL, 59.8 mmol, 10.0 eq) and N-methyl pyrrolidone (0.58 mL, 5.98 mmol, 1.00 eq) dropwise at 20° C. The reaction was stirred at 25° C. for 4 h. The reaction was carefully quenched with water (50.0 ml) and extracted with ethyl acetate (4×20.0 mL). The combined organic layers were washed with brine (20.0 mL), dried over sodium sulfate, filtered, and the filtrate was concentrated under reduced pressure to give a residue. The residue was purified by silica gel column chromatography (petroleum ether/ethyl acetate=70/30 to 60/40) to give 2-(chloromethyl)-1-methyl-4-nitrobenzene.

Step 3: To a mixture of 2-(chloromethyl)-1-methyl-4-nitrobenzene (500 mg, 2.69 mmol, 1.00 eq) in dimethylformamide (1.50 mL) and acetonitrile (1.50 mL) were added N,N-diisopropylethylamine (1.04 g, 8.08 mmol, 3.00 eq) and morpholine (258 mg, 2.96 mmol, 1.10 eq) dropwise at 20° C. The reaction was stirred at 60° C. for 10 h. The mixture was cooled to 20° C. and concentrated under reduced pressure to give a slurry, which was poured into water (50.0 mL) and the mixture was extracted with ethyl acetate (4×50.0 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=10/90 to 0/100) to afford 4-(2-methyl-5-nitrobenzyl)morpholine

Step 4: To a mixture of 4-(2-methyl-5-nitrobenzyl)morpholine (584 mg, 2.47 mmol, 1.00 eq) in ethanol (5.00 mL) and water (5.00 mL) were added ammonium chloride (132 mg, 2.47 mmol, 1.00 eq) and ferrous powder (690 mg, 12.4 mmol, 5.00 eq). The reaction was stirred at 90° C. for 10 h. The mixture was cooled to 20° C. and filtered. The filter cake was washed with methanol (2×20.0 mL). The filtrate was concentrated under reduced pressure to give a slurry. The slurry was poured into saturated aqueous sodium bicarbonate (50.0 mL) and extracted with ethyl acetate (4×50.0 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=10/90 to 0/100) to afford 4-methyl-3-(morpholinomethyl)aniline.

Step 5: To a mixture of 4-methyl-3-(morpholinomethyl)aniline (200 mg, 0.969 mmol, 1.50 eq) and triethylamine (98.0 mg, 0.969 mmol, 1.50 eq) in tetrahydrofuran (10.0 mL) was added 1,1′-carbonyldiimidazole (157 mg, 0.969 mmol, 1.50 eq). The reaction was stirred at 20° C. for 1 h. A mixture of 3-(6-(aminomethyl)-1-oxoisoindolin-2-yl)piperidine-2,6-dione hydrochloride $IV$ (200 mg, 0.646 mmol, 1.00 eq, hydrochloride) and triethylamine (196 mg, 1.94 mmol, 3.00 eq) in tetrahydrofuran (2.00 mL) was added. The reaction was stirred at 25° C. for 14 h. The mixture was quenched with water (50.0 ml) and extracted with ethyl acetate (4×50.0 mL). The combined organic layers were washed with brine (20.0 ml), dried over sodium sulfate, filtered, and concentrated and the obtained residue was purified by standard methodso afford 1-((2-(2,6-dioxopiperidin-3-yl)-3-oxoisoindolin-5-yl)methyl)-3-(4-methyl-3-(morpholinomethyl)phenyl)urea.

¹H NMR (400 MHz, DMSO-d₆) δ=10.98 (br s, 1H), 8.56 (s, 1H), 7.66 (s, 1H), 7.56 (d, J=0.8 Hz, 2H), 7.31-7.20 (m, 2H), 6.99 (d, J=8.0 Hz, 1H), 6.71 (t, J=6.0 Hz, 1H), 5.11 (dd, J=4.8, 13.2 Hz, TH), 4.38 (d, J=5.6 Hz, 2H), 4.37 (dd, J=17.2, 51.6 Hz, 2H), 3.55 (t, J=4.4 Hz, 4H), 3.34 (s, 2H), 2.91 (dddd, J=5.6, 9.6, 17.6, 31.2 Hz, 1H), 2.64-2.56 (m, 1H), 2.45-2.38 (m, 1H), 2.37-2.30 (m, 4H), 2.22 (s, 3H), 2.04-1.95 (m, 1H). LCMS m/z 506.5 [M+H]⁺

### Compound 102:

Step 1: To a solution of 3-nitro-1H-pyrazole (5.00 g, 44.2 mmol, 1.00 eq) in tetrahydrofuran (100 mL) was added sodium hydride 60% purity (2.13 g, 53.3 mmol, 1.20 eq) at 0° C. The reaction was stirred for 10 min, and 2-(trimethylsilyl)ethoxymethyl chloride (8.60 mL, 48.6 mmol, 1.10 eq) was added dropwise. The reaction was stirred at 20° C. for 1 h. The mixture was quenched with ice water (150 mL) and extracted with ethyl acetate (2×200 mL). The combined organic layers were washed with brine (200 mL), dried over sodium sulfate, filtered, and concentrated under reduced pressure to give a residue. The residue was purified by silica gel column chromatography (petroleum ether/ethyl acetate=10/1 to 1/1) to afford trimethyl-[2-[(3-nitropyrazol-1-yl)methoxy]ethyl]silane.

Step 2: To a solution of trimethyl-[2-[(3-nitropyrazol-1-yl)methoxy]ethyl]silane (3.00 g, 12.3 mmol, 1.00 eq) in methanol (50.0 mL) was added Pd/C 10% weight on C (0.50 g). The reaction was stirred at 30° C. for 4 h under hydrogen atmosphere (15 psi). The mixture was filtered, and the filtrate was concentrated under reduced pressure to afford 1-(2-trimethylsilylethoxymethyl)pyrazol-3-amine.

Step 3: To a solution of 1-(2-trimethylsilylethoxymethyl)pyrazol-3-amine (1.00 g, 4.69 mmol, 1.00 eq) and pyridine (0.76 mL, 9.37 mmol, 2.00 eq) in acetonitrile (8.00 mL) was added phenyl chloroformate (0.70 mL, 5.62 mmol, 1.20 eq) in acetonitrile (2.00 mL) at 0° C. The reaction was stirred at 25° C. for 3 h. The mixture was diluted with water (20.0 mL) and extracted with ethyl acetate (2×20.0 mL). The combined organic layers were dried over sodium sulfate, filtered, and concentrated under reduced pressure to give a residue. The residue was purified by silica gel column chromatography (petroleum ether/ethyl acetate=6/1) and concentrated under reduced pressure to afford phenyl (1-((2-(trimethylsilyl)ethoxy)methyl)-1H-pyrazol-3-yl)carbamate.

Step 4: To a solution of 3-[6-(aminomethyl)-1-oxo-isoindolin-2-yl]piperidine-2,6-dione hydrochloride VI-A (200 mg, 645 μmol, 1.00 eq, hydrochloride) and triethylamine (196 mg, 1.94 mmol, 267 μL, 3.00 eq) in dimethylformamide (3.00 mL) was added phenyl (1-((2-(trimethylsilyl)ethoxy)methyl)-1H-pyrazol-3-yl)carbamate (258 mg, 775 μmol, 1.20 eq). The reaction was stirred at 25° C. for 12 h. The mixture was diluted with water (20.0 mL) and extracted with dichloromethane/isopropyl alcohol=3/1 (3×15.0 mL). The combined organic layers were washed with brine (2×20.0 mL), dried over sodium sulfate, filtered, and concentrated under reduced pressure to afford 1-((2-(2,6-dioxopiperidin-3-yl)-3-oxoisoindolin-5-yl)methyl)-3-(1-((2-(trimethylsilyl)ethoxy)methyl)-1H-pyrazol-3-yl)urea.

Step 5: A mixture of 1-((2-(2,6-dioxopiperidin-3-yl)-3-oxoisoindolin-5-yl)methyl)-3-(1-((2-(trimethylsilyl)ethoxy) methyl)-1H-pyrazol-3-yl)urea (360 mg, 702 μmol, 1.00 eq) in trifluoroacetic acid (1.00 mL) and dichloromethane (1.00 mL) was stirred at 20° C. for 12 h. The mixture was concentrated and the obtained residue was purified by standard methods to afford. ¹H NMR (400 MHz, DMSO-d₆) δ=12.12 (br s, 1H), 10.99 (br s, 1H), 8.91 (s, 1H), 8.30 (br s, 1H), 7.64 (s, 1H), 7.56 (s, 2H), 7.53 (d, J=2.0 Hz, 1H), 7.44 (br s, 1H), 6.06 (s, 1H), 5.11 (dd, J=5.2, 13.2 Hz, 1H), 4.46-4.40 (m, 3H), 4.33-4.27 (m, 1H), 2.97-2.85 (m, 1H), 2.64-2.55 (m, 1H), 2.45-2.32 (m, 1H), 2.05-1.94 (m, 1H). MS (ESI) m/z 383.3 [M+H]⁺

Compound 103: General procedure A with variant iii) was used for the preparation from compound VI-A employing phenyl N-thiazol-5-ylcarbamate.

¹H NMR (400 MHz, DMSO-d₆) δ=1.96-2.03 (m, 1H) 2.39 (br dd, J=13.39, 4.71 Hz, 1H) 2.55-2.64 (m, 1H) 2.83-2.97 (m, 1H) 4.26-4.48 (m, 4H) 5.11 (dd, J=13.2, 5.2 Hz, 1H) 7.09-7.16 (m, 1H) 7.39 (d, J=0.8 Hz, 1H) 7.56 (s, 2H) 7.65 (s, 1H) 8.40 (s, 1H) 9.92 (br s, 1H) 10.91-11.05 (m, 1H). LCMS m/z 400.0 [M+H]⁺

### Preparation of phenyl N-thiazol-5-ylcarbamate

Step 1: To a vigorously stirred suspension of ethyl thiazole-5-carboxylate (1.84 g, 11.7 mmol, 1.00 eq) in methanol (11.0 mL) was added sodium hydroxide (1.40 g, 35.1 mmol, 3.00 eq). The reaction was stirred at 20° C. for 12 h. The mixture was concentrated under reduced pressure, and the remaining aqueous suspension was acidified with 6 M aqueous solution of hydrogen chloride to pH=1-2: The solid was filtered to afford thiazole-5-carboxylic acid.

Step 2: To a solution of thiazole-5-carboxylic acid (1.31 g, 10.2 mmol, 1.00 eq) in 1,4-dioxane (33.0 mL) was added triethylamine (1.61 mL, 11.59 mmol, 1.14 eq) and diphenylphosphoryl azide (2.51 mL, 11.6 mmol, 1.14 eq). The reaction was stirred at 20° C. for 3 h. Phenol (10.2 mL, 116.21 mmol, 11.43 eq) was added dropwise, and the reaction was stirred at 100° C. for 3 h. After cooling to 20° C., the mixture was concentrated under reduced pressure to give a residue. The residue was purified by silica gel column chromatography (petroleum ether/ethyl acetate=1/0 to 1/1) to afford phenyl N-thiazol-5-ylcarbamate.

Compound 104: General procedure A with variant iii) was used for the preparation from compound VI-A employing phenyl pyridin-4-ylcarbamate. ¹H NMR (400 MHz, DMSO-d₆) δ=10.98 (s, 1H), 9.14 (s, 1H), 8.29 (d, J=6.4 Hz, 2H), 8.14 (s, 1H), 7.67 (s, 1H), 7.57 (d, J=0.8 Hz, 2H), 7.43-7.37 (m, 2H), 7.02 (t, J=6.0 Hz, 1H), 5.11 (dd, J=4.8, 13.2 Hz, 1H), 4.415 (d, J=5.6 Hz, 2H), 4.37 (dd, J=17.2, 52.4 Hz, 2H), 2.96-2.84 (m, 1H), 2.65-2.56 (m, 1H), 2.43-2.34 (m, 1H), 2.05-1.95 (m, 1H). LCMS m/z 394.1 [M+H]⁺

Preparation of phenyl pyridin-4-ylcarbamate: A mixture of pyridin-4-amine (3.57 mL, 21.3 mmol, 1.00 eq), phenyl chloroformate (2.93 mL, 23.4 mmol, 1.10 eq) and triethylamine (5.92 mL, 42.5 mmol, 2.00 eq) in tetrahydrofuran (30.0 mL) was stirred at 20° C. for 0.5 h. The mixture was concentrated under reduced pressure to give a residue. The residue was purified by reversed phase column chromatography and lyophilized to afford phenyl pyridin-4-ylcarbamate.

Compound 105: To a solution of 3-(morpholinomethyl)aniline (279 mg, 1.45 mmol, 1.50 eq) and triethylamine (196 mg, 1.94 mmol, 2.00 eq) in dimethylformamide (5.00 mL) was added di(1H-imidazol-1-yl)methanone (236 mg, 1.45 mmol, 1.50 eq). The reaction was stirred at 20° C. for 2 h, then a solution of 3-(6-(aminomethyl)-1-oxo-isoindolin-2-yl)piperidine-2,6-dione hydrochloride VI-A (300 mg, 969 μmol, 1.00 eq, hydrochloride) and triethylamine (294 mg, 2.91 mmol, 3.00 eq) in dimethylformamide (5.00 mL) was added. The reaction was stirred at 20° C. for 12 h. The mixture was concentrated and the obtained residue was purified by standard methods to afford Compound 105. ¹H NMR (400 MHz, DMSO-d₆) δ=10.98 (s, 1H), 8.69 (s, 1H), 8.19 (s, 1H), 7.66 (s, 1H), 7.56 (d, J=0.8 Hz, 2H), 7.37 (s, 1H), 7.33-7.28 (m, 1H), 7.15 (t, J=7.6 Hz, 1H), 6.83 (d, J=7.6 Hz, 1H), 6.77 (t, J=6.0 Hz, 1H), 5.11 (dd, J=5.2, 13.2 Hz, 1H), 4.39 (d, J=5.6 Hz, 2H), 4.37 (dd, J=16.8, 52.0 Hz, 2H), 3.58-3.54 (m, 4H), 3.38 (s, 2H), 2.91 (ddd, J=5.2, 13.6, 17.2 Hz, 1H), 2.65-2.55 (m, 1H), 2.43-2.36 (m, 1H), 2.36-2.30 (m, 4H), 2.04-1.96 (m, 1H). LCMS m/z 492.2 [M+H]⁺

Compound 106: To a solution of 3-(difluoromethyl)aniline (416 mg, 2.91 mmol, 3.00 eq) and triethylamine (392 mg, 3.87 mmol, 4.00 eq) in dimethylformamide (10.0 mL) was added di(1H-imidazol-1-yl)methanone (518 mg, 3.20 mmol, 3.30 eq) in one portion at 20° C. The reaction was stirred at 20° C. for 1 h, then a solution of 3-[6-(aminomethyl)-1-oxo-isoindolin-2-yl]piperidine-2,6-dione hydrochloride VI-A (300 mg, 969 μmol, 1.00 eq, hydrochloride) and triethylamine (196 mg, 1.94 mmol, 2.00 eq) in dimethylformamide (1.00 mL) was added. The reaction was stirred at 20° C. for 12 h. The mixture was poured into saturated aqueous sodium bicarbonate (100 mL) and extracted with ethyl acetate (5×100 mL). The combined organic layers were dried over anhydrous sodium sulfate, filtered, and the filtrate was concentrated and the obtained residue was purified by standard methods to afford Compound 106. ¹H NMR (400 MHz, DMSO-d₆) δ=11.0 (s, 1H), 8.94 (s, 1H), 7.76 (s, 1H), 7.67 (s, 1H), 7.60-7.53 (m, 2H), 7.46 (dd, J=0.8, 8.0 Hz, 1H), 7.35 (t, J=8.0 Hz, 1H), 7.10-7.06 (m, 1H), 6.96 (s, 1H), 6.88 (t, J=6.0 Hz, 1H), 5.11 (dd, J=4.8, 13.2 Hz, 1H), 4.41 (s, 2H), 4.36 (dd, J=17.2, 52.0 Hz, 2H), 2.97-2.85 (m, 1H), 2.64-2.55 (m, 1H), 2.45-2.36 (m, 1H), 2.05-1.95 (m, 1H). LCMS m/z 443.1 [M+H]⁺

Compound 107: General procedure A with variant iv) was used for the preparation from compound VI-A employing 2-chloro-4-isocyanato-1,3,5-trimethylbenzene. ¹H NMR (400 MHz, DMSO-d₆) δ=11.0 (br s, 1H), 7.79 (s, 1H), 7.66 (s, 1H), 7.58-7.45 (m, 2H), 7.05 (s, 1H), 6.86-6.60 (m, 1H), 5.10 (dd, J=5.1, 13.3 Hz, 1H), 4.50-4.20 (m, 4H), 2.99-2.82 (m, 1H), 2.65-2.56 (m, 1H), 2.39 (dq, J=4.3, 13.2 Hz, 1H), 2.27 (s, 3H), 2.20 (s, 3H), 2.12 (s, 3H), 2.05-1.93 (m, 1H). MS (ESI) m/z 469.2 [M+H]⁺

### Preparation of 2-chloro-4-isocyanato-1,3,5-trimethylbenzene

Step 1: To a solution of 2-chloro-1,3,5-trimethylbenzene (2.9 g, 18.8 mmol, 1.00 eq) in acetic anhydride (21.0 mL) was added a solution of nitric acid (2.53 mL, 56.3 mmol, 3.00 eq) in acetic anhydride (10.5 mL) at −78° C. The reaction was stirred at −78° C. for 2 h. Water (50.0 mL) was added to quench the reaction, and the pH was adjusted to pH=7 with an aqueous solution of sodium bicarbonate. The mixture was extracted with ethyl acetate (2×100 mL). The combined organic layers were washed with brine (2×50.0 mL), dried over sodium sulfate, filtered, and concentrated under reduced pressure to give a residue. The residue was purified by silica gel column chromatography (petroleum ether/ethyl acetate=1/0 to 10/1) to give 2-chloro-1,3,5-trimethyl-4-nitrobenzene.

Step 2: To a solution of 2-chloro-1,3,5-trimethyl-4-nitrobenzene (950 mg, 4.76 mmol, 1.00 eq) in ethanol (10.0 mL) and water (3.00 mL) was added iron powder (797 mg, 14.3 mmol, 3.00 eq) and ammonium chloride (1.27 g, 23.8 mmol, 5.00 eq). The reaction was stirred at 80° C. for 12 h. The mixture was filtered and concentrated under reduced pressure to give a residue. Water (50.0 mL) was added, and the pH was adjusted to pH=7 with an aqueous solution of sodium bicarbonate. The mixture was extracted with ethyl acetate (2×100 mL). The combined organic layers were washed with brine (2×50.0 mL), dried over sodium sulfate, filtered, and concentrated under reduced pressure to afford 3-chloro-2,4,6-trimethylaniline.

Step 3: To a solution of 3-chloro-2,4,6-trimethylaniline (300 mg, 1.77 mmol, 1.00 eq) in toluene (10.0 mL) was added triphosgene (525 mg, 1.77 mmol, 1.00 eq). The reaction was stirred at 100° C. for 2 h. The mixture was concentrated to afford 2-chloro-4-isocyanato-1,3,5-trimethylbenzene.

### Compound 108:

Step 1: To a solution of tert-butyl 4-aminopyrazole-1-carboxylate (500 mg, 2.73 mmol, 1.00 eq), pyridine (0.44 mL, 5.46 mmol, 2.00 eq) in acetonitrile (5.00 mL) was added phenyl chloroformate (0.41 mL, 3.27 mmol, 1.20 eq) at 0° C. The reaction was stirred at 30° C. for 2 h. The mixture was diluted with water (15.0 mL) and extracted with ethyl acetate (2×20.0 mL). The combined organic layers were washed with brine (20 mL), dried over sodium sulfate, filtered, and concentrated under reduced pressure to give a residue. The residue was purified by silica gel column chromatography (petroleum ether/ethyl acetate=10/1 to 5/1) and concentrated under reduced pressure to afford tert-butyl 4-(phenoxycarbonylamino)pyrazole-1-carboxylate.

Step 2: To a mixture of 3-[6-(aminomethyl)-1-oxo-isoindolin-2-yl]piperidine-2,6-dione hydrochloride VI-A (500 mg, 1.61 mmol, 1.00 eq, hydrochloride) and triethylamine (674 μL, 4.84 mmol, 3.00 eq) in dimethylformamide (5.00 mL) was added tert-butyl 4-(phenoxycarbonylamino)pyrazole-1-carboxylate (587 mg, 1.94 mmol, 1.20 eq). The reaction was stirred at 30° C. for 6 h. The mixture was diluted with water (20.0 mL) and extracted with dichloromethane/isopropyl alcohol=3/1 (3×30.0 mL). The combined organic layers were washed with brine (20.0 mL), dried over sodium sulfate, filtered, and concentrated under reduced pressure to give a residue. The residue was purified by silica gel column chromatography (petroleum ether/ethyl acetate=3/1 to dichloromethane/methanol=10/1) and concentrated under reduced pressure to afford tert-butyl 4-[[2-(2,6-dioxo-3-piperidyl)-3-oxo-isoindolin-5-yl]methylcarbamoylamino]pyrazole-1-carboxylate.

Step 3: To a solution of tert-butyl 4-[[2-(2,6-dioxo-3-piperidyl)-3-oxo-isoindolin-5-yl]methylcarbamoylamino]pyrazole-1-carboxylate (500 mg, 1.04 mmol, 1.00 eq) in dioxane (5.00 mL) was added hydrochloric acid/dioxane (4 M, 2.5 mL, 9.65 eq). The reaction was stirred at 25° C. for 1 h. The mixture was concentrated and the obtained residue was purified by standard methods to afford Compound 108.

¹H NMR (400 MHz, DMSO-d₆) δ=12.37 (br s, 1H), 10.99 (br s, 1H), 8.28 (s, 1H), 7.64 (s, 1H), 7.54 (s, 2H), 7.50 (br s, 1H), 6.68 (br t, J=5.6 Hz, 1H), 5.11 (dd, J=5.2, 13.3 Hz, 1H), 4.47-4.26 (m, 4H), 2.97-2.85 (m, 1H), 2.64-2.55 (m, 1H), 2.45-2.33 (m, 1H), 2.04-1.94 (m, 1H). MS (ESI) m/z 383.0 [M+H]⁺

Compound 109: General procedure A with variant iii) was used for the preparation from compound VI-A employing phenyl (3-(2-oxa-6-azaspiro[3.3]heptan-6-ylmethyl)-5-chloro-4-methylphenyl)carbamate. ¹H NMR (400 MHz, DMSO-d₆) δ=11.0 (br s, 1H), 8.91-8.62 (m, 1H), 7.66 (s, 1H), 7.62 (d, J=2.1 Hz, 1H), 7.56 (s, 2H), 7.06 (d, J=2.0 Hz, 1H), 6.88-6.69 (m, 1H), 5.11 (dd, J=5.0, 13.2 Hz, 1H), 4.60 (s, 4H), 4.48-4.27 (m, 4H), 3.44 (s, 2H), 3.30 (s, 4H), 2.97-2.84 (m, 1H), 2.59 (td, J=2.0, 15.2 Hz, 1H), 2.41 (br dd, J=8.9, 13.3 Hz, 1H), 2.17 (s, 3H), 2.04-1.93 (m, 1H). MS (ESI) m/z 552.2 [M+H]⁺

### Preparation of phenyl (3-(2-oxa-6-azaspiro[3.3]heptan-6-ylmethyl)-5-chloro-4-methylphenyl)carbamate

Step 1: To a solution of (3-chloro-2-methyl-5-nitrophenyl)methanol (1.50 g, 7.44 mmol, 1.00 eq) in dichloromethane (20.0 mL) was added thionyl chloride (2.70 mL, 37.2 mmol, 5.00 eq) at 0° C. The reaction was stirred at 25° C. for 12 h. The mixture was concentrated under reduced pressure to give a residue. The residue was purified by silica gel column chromatography (petroleum ether/ethyl acetate=1/0 to 10/1) to afford 1-chloro-3-(chloromethyl)-2-methyl-5-nitrobenzene.

Step 2: To a solution of 1-chloro-3-(chloromethyl)-2-methyl-5-nitrobenzene (900 mg, 4.09 mmol, 1.00 eq) in dimethylformamide (2.00 mL) was added potassium carbonate (1.13 g, 8.18 mmol, 2.00 eq), potassium iodide (67.9 mg, 0.41 mmol, 0.100 eq) and 2-oxa-6-azaspiro[3.3]heptane (811 mg, 8.18 mmol, 2.00 eq). The reaction was stirred at 20° C. for 12 h. The mixture was filtered and concentrated under reduced pressure to give a residue. The residue was purified by silica gel column chromatography (petroleum ether/ethyl acetate=1/0 to 3/1) to afford 6-(3-chloro-2-methyl-5-nitrobenzyl)-2-oxa-6-azaspiro[3.3]heptane.

Step 3: To a solution of 6-(3-chloro-2-methyl-5-nitrobenzyl)-2-oxa-6-azaspiro[3.3]heptane (600 mg, 2.12 mmol, 1.00 eq) in ethanol (6.00 mL) and water (2.00 mL) was added iron powder (356 mg, 6.37 mmol, 3.00 eq) and ammonium chloride (568 mg, 10.6 mmol, 5.00 eq). The reaction was stirred at 80° C. for 2 h. The mixture was filtered and concentrated under reduced pressure to give a residue. Ethyl acetate (50.0 mL) was added to the residue, and the mixture was washed with a solution of saturated sodium bicarbonate (2×30.0 mL) and water (2×30.0 mL). The organic layer was dried over sodium sulfate, filtered, and concentrated under reduced pressure to afford 3-(2-oxa-6-azaspiro[3.3]heptan-6-ylmethyl)-5-chloro-4-methylaniline.

Step 4: To a solution of 3-(2-oxa-6-azaspiro[3.3]heptan-6-ylmethyl)-5-chloro-4-methylaniline (140 mg, 554 μmol, 1.00 eq) in dichloromethane (2.00 mL) was added pyridine (0.27 mL, 3.32 mmol, 6.00 eq) and a solution of phenyl chloroformate (69.4 μL, 554 μmol, 1.00 eq) in dichloromethane (2.00 mL) dropwise at 0° C. The reaction was stirred at 0° C. for 1 h. The mixture was concentrated under reduced pressure to give a residue. The residue was purified by silica gel column chromatography (dichloromethane/methanol=1/0 to 10/1) to afford phenyl (3-(2-oxa-6-azaspiro[3.3]heptan-6-ylmethyl)-5-chloro-4-methylphenyl)carbamate.

### Compound 110:

Step 1: To a mixture of 3-aminophenol (1.00 g, 9.16 mmol, 1.00 eq) and sodium bicarbonate (0.43 mL, 11.0 mmol, 1.20 eq) in tetrahydrofuran (10.0 mL) and water (1.00 mL) was added phenyl chloroformate (1.21 mL, 9.62 mmol, 1.05 eq) at 0° C. The reaction was stirred at 0° C. for 2 h. The mixture was quenched with water (10.0 mL) and extracted with ethyl acetate (10.0 mL). The organic phase was dried over sodium sulfate, filtered, and concentrated under reduced pressure to afford phenyl N-(3-hydroxyphenyl)carbamate.

Step 2: A mixture of 3-[6-(aminomethyl)-1-oxo-isoindolin-2-yl]piperidine-2,6-dione hydrochloride VI-A (0.800 g, 2.58 mmol, 1.00 eq, hydrochloride), phenyl N-(3-hydroxyphenyl)carbamate (651 mg, 2.84 mmol, 1.10 eq) and triethylamine (1.08 mL, 7.75 mmol, 3.00 eq) in dimethyl formamide (10.0 mL) was heated to 50° C. for 2 h. The mixture was added dropwise to ethyl acetate (50.0 mL) at 0° C., and the resulting solid was filtered, washed with ethyl acetate (2×3 mL) and dried to afford 1-[[2-(2,6-dioxo-3-piperidyl)-3-oxo-isoindolin-5-yl]methyl]-3-(3-hydroxyphenyl)urea.

Step 3: To a mixture of 2-morpholinoacetic acid (89 mg, 612 μmol, 1.00 eq) and N,N-dimethylpyridin-4-amine (7.5 mg, 61.2 μmol, 0.10 eq) in dimethyl formamide (3.00 mL) was added N,N′-methanediylidenedicyclohexanamine (136 μL, 673 μmol, 1.10 eq) at 0° C. The reaction was stirred at 0° C. for 30 min, then 1-[[2-(2,6-dioxo-3-piperidyl)-3-oxo-isoindolin-5-yl]methyl]-3-(3-hydroxyphenyl) urea (250 mg, 612 μmol, 1.00 eq) was added.

The reaction was stirred at 20° C. for 16 h. The mixture was filtered, and the filtrate was concentrated and the obtained residue was purified by standard methods to afford Compound 110.

1H NMR (400 MHz, DMSO-d₆) δ=10.99 (s, 1H), 8.83 (s, 1H), 7.67 (s, 1H), 7.57 (s, 2H), 7.40 (s, 1H), 7.29-7.21 (m, 1H), 7.14 (br d, J=8.1 Hz, 1H), 6.81 (br t, J=5.6 Hz, 1H), 6.66 (br d, J=7.9 Hz, 1H), 5.12 (br dd, J=13.3, 5.0 Hz, 1H), 4.49-4.36 (m, 3H), 4.35-4.26 (m, 1H), 3.64-3.56 (m, 4H), 3.51 (s, 2H), 2.97-2.85 (m, 1H), 2.62 (br s, 1H), 2.58 (br d, J=4.0 Hz, 4H), 2.46-2.35 (m, 1H), 2.05-1.95 (m, 1H). MS (ESI) m/z 536.2 [M+H]⁺

### Compound 111:

Step 1: A mixture of tert-butyl 4-((methylsulfonyl)oxy)piperidine-1-carboxylate (2.41 g, 8.63 mmol, 1.20 eq), 3-nitrophenol (1.43 mL, 7.19 mmol, 1.00 eq), potassium carbonate (1.29 g, 9.35 mmol, 1.30 eq) in anhydrous dimethylformamide (20.0 mL) was stirred at 80° C. for 8 h under nitrogen. The mixture was diluted with water (20.0 mL) and extracted with ethyl acetate (3×10.0 mL). The combined organic layers were washed with brine (10.0 mL), dried over anhydrous sodium sulfate, filtered, and concentrated under reduced pressure to give a residue. The residue was purified by silica gel column chromatography (petroleum ether/ethyl acetate=10/1 to 7/1) to afford tert-butyl 4-(3-nitrophenoxy)piperidine-1-carboxylate.

Step 2: To a solution of tert-butyl 4-(3-nitrophenoxy)piperidine-1-carboxylate (500 mg, 1.55 mmol, 1.00 eq) in ethanol (10.0 mL) was added Pd/C 10% weight on C (400 mg, 1.00 eq) under hydrogen atmosphere (15 psi). The reaction was stirred at 25° C. for 6 h. The mixture was filtered, and the filtrate was concentrated under reduced pressure to afford tert-butyl 4-(3-aminophenoxy)piperidine-1-carboxylate.

Step 3: To a solution of tert-butyl 4-(3-aminophenoxy)piperidine-1-carboxylate (428 mg, 1.46 mmol, 1.00 eq) in dichloromethane (5.00 mL) was added pyridine (0.15 mL, 1.90 mmol, 1.30 eq) and phenyl chloroformate (0.20 mL, 1.61 mmol, 1.10 eq) at 0° C. The reaction was stirred at 25° C. for 3 h. The mixture was diluted with water (5.00 mL) and extracted with ethyl acetate (3×5.00 mL). The combined organic layers were washed with brine (5.00 mL), dried over anhydrous sodium sulfate, filtered, and concentrated under reduced pressure to afford tert-butyl 4-(3-((phenoxycarbonyl)amino)phenoxy) piperidine-1-carboxylate.

Step 4: To a solution of tert-butyl 4-(3-((phenoxycarbonyl)amino)phenoxy)piperidine-1-carboxylate (399 mg, 968 μmol, 1.20 eq) in dimethyl formamide (4.00 mL) was added 3-[6-(aminomethyl)-1-oxo-isoindolin-2-yl]piperidine-2,6-dione hydrochloride VI-A (250 mg, 807 μmol, 1.00 eq, hydrochloride) and triethylamine (727 mg, 7.18 mmol, 1.00 mL, 8.90 eq). The reaction was stirred at 40° C. for 2 h under nitrogen. The mixture was diluted with water (10.0 mL) and extracted with ethyl acetate (3×15.0 mL). The combined organic layers were washed with brine (10.0 mL), dried over anhydrous sodium sulfate, filtered, and concentrated under reduced pressure to give a residue. The residue was purified by silica gel column chromatography (dichloromethane/methanol=25/1 to 20/1) to give tert-butyl 4-(3-(3-((2-(2,6-dioxopiperidin-3-yl)-3-oxoisoindolin-5-yl)methyl)ureido)phenoxy)piperidine-1-carboxylate.

Step 5: A solution of tert-butyl 4-(3-(3-((2-(2,6-dioxopiperidin-3-yl)-3-oxoisoindolin-5-yl)methyl)ureido)phenoxy)piperidine-1-carboxylate (210 mg, 354 μmol, 1.00 eq) in hydrochloric acid/ethyl acetate (4.00 mL) was stirred at 20° C. for 0.5 h. The mixture was concentrated and the obtained residue was purified by standard methods to afford Compound 111.

¹H NMR (400 MHz, DMSO-d₆) δ=10.98 (s, 1H), 8.82 (br s, 1H), 8.68 (br s, 2H), 7.64 (s, 1H), 7.55 (s, 2H), 7.27 (s, 1H), 7.12 (t, J=8.1 Hz, 1H), 6.89 (br s, 1H), 6.84 (br d, J=7.9 Hz, 1H), 6.54 (dd, J=2.1, 8.2 Hz, 1H), 5.11 (dd, J=5.0, 13.3 Hz, 1H), 4.56 (br d, J=3.3 Hz, 1H), 4.46-4.36 (m, 3H), 4.33-4.26 (m, 1H), 3.24-3.15 (m, 2H), 3.11-3.01 (m, 2H), 2.96-2.84 (m, 1H), 2.59 (br d, J=18.3 Hz, 1H), 2.42-2.30 (m, 1H), 2.10-1.95 (m, 3H), 1.83 (br d, J=3.5 Hz, 2H). MS (ESI) m/z 492.2 [M+H]⁺

Compound 112: General procedure A with variant iii) was used for the preparation from compound VI-A employing phenyl (3-(1H-imidazol-2-yl)phenyl)carbamate. ¹H NMR (400 MHz, DMSO-d₆) δ=11.00 (br s, 1H), 8.80 (s, 1H), 8.18 (s, 1H), 7.98 (s, 1H), 7.68 (s, 1H), 7.61-7.52 (m, 2H), 7.46-7.36 (m, 2H), 7.31-7.23 (m, 1H), 7.09 (br s, 2H), 6.86 (t, J=6.0 Hz, 1H), 5.12 (dd, J=5.1, 13.3 Hz, 1H), 4.48-4.37 (m, 3H), 4.34-4.25 (m, 1H), 2.97-2.83 (m, 1H), 2.58 (br dd, J=2.2, 15.3 Hz, 1H), 2.42-2.33 (m, 1H), 2.03-1.94 (m, 1H). MS (ESI) m/z 459.1[M+H]⁺

### Preparation of phenyl (3-(1H-imidazol-2-yl)phenyl)carbamate

Step 1: To a mixture of 2-(3-nitrophenyl)-1H-imidazole (900 mg, 4.76 mmol, 1.00 eq), di-tert-butyl dicarbonate (1.64 mL, 7.14 mmol, 1.50 eq), and 4-dimethylaminopyridine (58.1 mg, 475 μmol, 0.10 eq) in dichloromethane (9.00 mL) was added triethylamine (993 μL, 7.14 mmol, 1.50 eq) and the reaction was stirred for 16 h at 25° C. The mixture was diluted with water (10.0 mL) and extracted with dichloromethane (3×10.0 mL). The combined organic layers were washed with brine (10.0 mL), dried over anhydrous sodium sulfate, filtered, and concentrated under reduced pressure to give a residue. The residue was purified by silica gel column chromatography (petroleum ether/ethyl acetate=10/1 to 1/1) to give tert-butyl 2-(3-nitrophenyl)-1H-imidazole-1-carboxylate.

Step 2: To a solution of tert-butyl 2-(3-nitrophenyl)-1H-imidazole-1-carboxylate (500 mg, 1.73 mmol, 1.00 eq) in ethyl alcohol (10.0 mL) was added Pd/C 10% weight on C (400 mg, 1.73 mmol, 1.00 eq), and the mixture was stirred at 25° C. for 3 h under hydrogen atmosphere (15 psi). The mixture was filtered, and the filtrate was concentrated under reduced pressure to give tert-butyl 2-(3-aminophenyl)-1H-imidazole-1-carboxylate.

Step 3: To a solution of tert-butyl 2-(3-aminophenyl)-1H-imidazole-1-carboxylate (790 mg, 3.05 mmol, 1.00 eq) in dichloromethane (10.0 mL) was added pyridine (0.32 mL, 3.96 mmol, 1.30 eq) and phenyl chloroformate (524 mg, 3.35 mmol, 419 μL, 1.10 eq) at 0° C. The reaction was stirred for 3 h at 25° C. The mixture was diluted with water (50.0 mL) and extracted with dichloromethane (3×50.0 mL). The combined organic layers were washed with brine (20.0 mL), dried over anhydrous sodium sulfate, filtered, and concentrated under reduced pressure to give phenyl (3-(1H-imidazol-2-yl)phenyl)carbamate.

Compound 113: General procedure A with variant iii) was used for the preparation from compound VI-A employing phenyl (3-(2-methyl-1H-imidazol-1-yl)phenyl)carbamate. ¹H NMR (400 MHz, DMSO-d₆) δ=10.99 (s, 1H), 8.96 (s, 1H), 8.15 (s, 1H), 7.67 (s, 1H), 7.60 (s, 1H), 7.59-7.54 (m, 2H), 7.37 (d, J=4.6 Hz, 2H), 7.26 (s, 1H), 6.99-6.88 (m, 3H), 5.11 (dd, J=5.1, 13.5 Hz, 1H), 4.46-4.38 (m, 3H), 4.33-4.28 (m, 1H), 2.97-2.85 (m, 1H), 2.60 (br d, J=17.2 Hz, 1H), 2.39 (dq, J=4.3, 13.3 Hz, 1H), 2.28 (s, 3H), 2.05-1.94 (m, 1H). MS (ESI) m/z 473.1 [M+H]⁺

Preparation of phenyl (3-(2-methyl-1H-imidazol-1-yl)phenyl)carbamate: To a solution of 3-(2-methylimidazol-1-yl)aniline (90.0 mg, 520 μmol, 1.00 eq) in dichloromethane (3.00 mL) was added pyridine (84 μL, 1.04 mmol, 2.00 eq). Phenyl chloroformate (72 μL, 572 μmol, 1.10 eq) was added to the mixture portion-wise. The reaction was stirred at 20° C. for 2 h. The mixture was poured into water (5.00 mL) and extracted with dichloromethane (2×5.00 m). The combined organic layers were concentrated and the obtained residue was purified by standard methods to afford phenyl (3-(2-methyl-1H-imidazol-1-yl)phenyl)carbamate.

Compound 114: General procedure A with variant iii) was used for the preparation from compound VI-A employing phenyl (4-(difluoromethyl)phenyl)carbamate. ¹H NMR (400 MHz, DMSO-d₆) δ=10.98 (s, 1H), 8.93 (s, 1H), 7.67 (s, 1H), 7.58-7.48 (m, 4H), 7.42 (d, J=8.4 Hz, 2H), 7.07-6.73 (m, 2H), 5.11 (dd, J=5.1, 13.3 Hz, 1H), 4.48-4.37 (m, 3H), 4.36-4.25 (m, 1H), 2.91 (ddd, J=5.4, 13.5, 17.5 Hz, 1H), 2.62-2.57 (m, 1H), 2.39 (br dd, J=4.5, 13.1 Hz, 1H), 2.04-1.95 (m, 1H). MS (ESI) m/z 423.3 [M−20]⁺

Preparation of phenyl (4-(difluoromethyl)phenyl)carbamate: To a solution of 4-(difluoromethyl)aniline hydrochloride (400 mg, 2.23 mmol, 1.00 eq) in tetrahydrofuran (8.00 mL) was added triethylamine (0.62 mL, 4.45 mmol, 2.00 eq) and phenyl chloroformate (0.31 mL, 2.45 mmol, 1.10 eq). The reaction was stirred at 20° C. for 1 h. The mixture was filtered and concentrated and the obtained residue was purified by standard methods to give phenyl (4-(difluoromethyl)phenyl)carbamate.

Compound 115: General procedure A with variant iii) was used for the preparation from compound VI-A employing phenyl (3-(1-methyl-1H-pyrazol-5-yl)phenyl)carbamate. ¹H NMR (400 MHz, DMSO-d₆) δ=10.98 (s, 1H), 8.81 (s, 1H), 7.67 (s, 1H), 7.61 (s, 1H), 7.59-7.55 (m, 2H), 7.46-7.41 (m, 2H), 7.37-7.31 (m, 1H), 7.05 (d, J=7.9 Hz, 1H), 6.84 (t, J=6.1 Hz, 1H), 6.35 (d, J=1.8 Hz, 1H), 5.11 (dd, J=5.1, 13.2 Hz, 1H), 4.47-4.39 (m, 3H), 4.34-4.27 (m, 1H), 3.83 (s, 3H), 2.97-2.86 (m, 1H), 2.60 (br d, J=15.4 Hz, 1H), 2.44-2.31 (m, 1H), 2.04-1.96 (m, 1H). MS (ESI) m/z 473.1 [M+H]⁺

### Preparation of phenyl (3-(1-methyl-1H-pyrazol-5-yl)phenyl)carbamate

Step 1: To a mixture of 5-bromo-1-methyl-pyrazole (0.500 g, 3.11 mmol, 1.00 eq) and (3-aminophenyl)boronic acid (510 mg, 3.73 mmol, 1.20 eq) in dioxane (10.0 mL) and water (1.00 mL) was added tetrakis(triphenylphosphine)palladium (359 mg, 311 μmol, 0.10 eq) and potassium phosphate (1.98 g, 9.32 mmol, 3.0 eq). The reaction was stirred at 110° C. for 16 h. The mixture was poured into water (20.0 mL), and the product was extracted with ethyl acetate (2×20.0 mL). The combined organic layers were washed with brine (10.0 mL) and concentrated under reduced pressure to give a residue. The residue was purified by silica gel column chromatography (petroleum ether/ethyl acetate=5/1 to 1/1) to afford 3-(2-methylpyrazol-3-yl)aniline.

Step 2: To a solution of 3-(2-methylpyrazol-3-yl)aniline (0.400 g, 2.31 mmol, 1.00 eq) in dichloromethane (5.00 mL) was added pyridine (0.34 mL, 4.62 mmol, 2.00 eq). The mixture was cooled to 0° C., and phenyl chloroformate (0.32 mL, 2.54 mmol, 1.10 eq) was added dropwise. The reaction was stirred at 20° C. for 1 h. Water (5.00 mL) was added to the mixture. The organic layer was separated and washed with brine (5.00 mL), dried over anhydrous sodium sulfate, filtered, and concentrated to give phenyl (3-(1-methyl-1H-pyrazol-5-yl)phenyl)carbamate (650 mg, crude).

Compound 116: General procedure A with variant iii) was used for the preparation from compound VI-A employing phenyl N-[3-(1-methylpyrazol-3-yl)phenyl]carbamate. ¹H NMR (400 MHz, DMSO-d₆) δ ppm 10.98 (br s, 1H), 8.70 (br s, 1H), 7.89 (br s, 1H), 7.76-7.65 (m, 2H), 7.58 (br s, 2H), 7.39-7.18 (m, 3H), 6.74 (br s, 1H), 6.58 (br d, J=2.1 Hz, 1H), 5.12 (br dd, J=13.1, 4.9 Hz, 1H), 4.49-4.26 (m, 4H), 3.88 (s, 3H), 2.99-2.83 (m, 1H), 2.60 (br d, J=17.4 Hz, 1H), 2.45-2.36 (m, 1H), 2.07-1.94 (m, 1H). MS (ESI) m/z 473.2 [M+H]⁺

### Preparation of phenyl N-[3-(1-methylpyrazol-3-yl)phenyl]carbamate

Step 1: A mixture of 3-bromo-1-methyl-pyrazole (0.550 g, 3.42 mmol, 1.00 eq), (3-aminophenyl)boronic acid (561 mg, 4.10 mmol, 1.20 eq), potassium phosphate (2.18 g, 10.3 mmol, 3.00 eq) and tetrakis(triphenylphosphine)palladium (197 mg, 171 μmol, 0.05 eq) in dioxane (10.0 mL) and water (1.00 mL) was heated to 110° C. for 16 h under nitrogen. Water (10.0 mL) was added to the mixture, and it was extracted with ethyl acetate (2×10.0 mL). The combined organic layers were washed with brine (10.0 mL), dried over sodium sulfate, filtered, and concentrated under reduced pressure to give a residue. The residue was purified by reversed phase preparative HPLC to give 3-(1-methylpyrazol-3-yl)aniline.

Step 2: To a solution of 3-(1-methylpyrazol-3-yl)aniline (0.380 g, 2.19 mmol, 1.00 eq) and pyridine (0.53 mL, 6.58 mmol, 3.00 eq) in dichloromethane (10.0 mL) at 0° C. was added phenyl chloroformate (0.30 mL, 2.41 mmol, 1.10 eq). The mixture was allowed to warm to 20° C. and stirred for 2 h. Water (10.0 mL) was added, and the organic layer was separated, dried over sodium sulfate, filtered and concentrated to give phenyl N-[3-(1-methylpyrazol-3-yl)phenyl]carbamate.

Compound 117: General procedure A with variant iii) was used for the preparation from compound VI-A employing (3-chloro-5-(3-(dimethylamino)propoxy)-4-methylphenyl)carbamate. ¹H NMR (400 MHz, DMSO-d₆) δ=10.99 (s, 1H), 8.91 (s, 1H), 8.23 (s, 1H), 7.66 (s, 1H), 7.56 (d, J=1.0 Hz, 2H), 7.15 (d, J=1.9 Hz, 1H), 7.05 (d, J=1.8 Hz, 1H), 7.03-6.89 (m, 1H), 5.12 (dd, J=5.1, 13.3 Hz, 1H), 4.42-4.30 (m, 4H), 3.98-3.94 (m, 2H), 2.95-2.88 (m, 1H), 2.63-2.58 (m, 1H), 2.47-2.44 (m, 2H), 2.42-2.36 (m, 1H), 2.21 (s, 6H), 2.11 (s, 3H), 2.03-1.97 (m, 1H), 1.89 (t, J=6.8 Hz, 2H). MS (ESI) m/z 542.2 [M+H]⁺

### Preparation of (3-chloro-5-(3-(dimethylamino)propoxy)-4-methylphenyl)carbamate

Step 1: A mixture of methyl 3-chloro-5-hydroxy-4-methyl-benzoate (1.00 g, 4.98 mmol, 1.00 eq), 3-chloro-N,N-dimethyl-propan-1-amine hydrochloride (709 mg, 4.49 mmol, 0.90 eq, hydrochloride) and potassium carbonate (2.07 g, 14.9 mmol, 3.00 eq) in acetonitrile (20.0 mL) was stirred at 85° C. for 12 h. The mixture was diluted with water (50.0 mL) and extracted with ethyl acetate (3×20.0 mL). The combined organic layers were washed with brine (3×10.0 mL), dried over sodium sulfate, filtered, and concentrated under reduced pressure to give a residue. The residue was purified by reversed phase preparative HPLC to give methyl 3-chloro-5-(3-(dimethylamino)propoxy)-4-methylbenzoate.

Step 2: To a solution of methyl 3-chloro-5-(3-(dimethylamino)propoxy)-4-methylbenzoate (900 mg, 3.15 mmol, 1.00 eq) in water (10.0 mL) and methanol (20.0 mL) was added sodium hydroxide (252 mg, 6.30 mmol, 2.00 eq). The reaction was stirred at 60° C. for 2 h. The mixture was concentrated under reduced pressure to give a residue. The residue was purified by reversed phase preparative HPLC to afford 3-chloro-5-(3-(dimethylamino)propoxy)-4-methylbenzoic acid.

Step 3: To a mixture of 3-chloro-5-(3-(dimethylamino)propoxy)-4-methylbenzoic acid (600 mg, 2.21 mmol, 1.00 eq) and triethylamine (614 μL, 4.42 mmol, 2.00 eq) in 2-methylpropan-2-ol (10.0 mL) was added diphenyl phosphoryl azide (0.96 mL, 4.42 mmol, 2.00 eq). The reaction was stirred at 100° C. for 12 h. The mixture was concentrated under reduced pressure to give a residue. The residue was purified by reversed phase preparative HPLC to afford tert-butyl (3-chloro-5-(3-(dimethylamino)propoxy)-4-methylphenyl)carbamate.

Step 4: A solution of tert-butyl (3-chloro-5-(3-(dimethylamino)propoxy)-4-methylphenyl)carbamate (350 mg, 1.02 mmol, 1.00 eq) in hydrogen chloride/ethyl acetate (4.00 M, 10.0 mL, 39.2 eq) was stirred at 25° C. for 0.5 h. The mixture was concentrated under reduced pressure to give 3-chloro-5-(3-(dimethylamino)propoxy)-4-methylaniline.

Step 5: To a solution of 3-chloro-5-(3-(dimethylamino)propoxy)-4-methylaniline (300 mg, 1.07 mmol, 1.00 eq, hydrogen chloride) and triethylamine (326 mg, 3.22 mmol, 3.00 eq) in dichloromethane (5.00 mL) was added phenyl chloroformate (252 mg, 1.61 mmol, 1.50 eq) at 25° C. The mixture was stirred at 25° C. for 0.5 h. The mixture was concentrated and the obtained residue was purified by standard methods to give phenyl (3-chloro-5-(3-(dimethylamino)propoxy)-4-methylphenyl)carbamate.

Compound 118: General procedure A with variant iii) was used for the preparation from compound VI-A employing phenyl N-[3,4-dimethyl-5-(morpholinomethyl) phenyl]carbamate. ¹H NMR (400 MHz, DMSO-d₆) δ=10.98 (s, 1H), 8.43 (s, 1H), 8.15 (s, 1H), 7.65 (s, 1H), 7.55 (d, J=0.8 Hz, 2H), 7.17 (d, J=2.0 Hz, 1H), 7.10 (d, J=2.0 Hz, 1H), 6.65 (t, J=6.0 Hz, 1H), 5.11 (dd, J=5.2, 13.2 Hz, 1H), 4.46-4.28 (m, 4H), 3.54 (br t, J=4.0 Hz, 4H), 3.34 (s, 2H), 2.97-2.85 (m, 1H), 2.59 (br d, J=17.4 Hz, 1H), 2.45-2.38 (m, 1H), 2.34 (br s, 4H), 2.17 (s, 3H), 2.12 (s, 3H), 2.04-1.95 (m, 1H). MS (ESI) m/z 520.2 [M+H]⁺

### Preparation of phenyl N-[3,4-dimethyl-5-(morpholinomethyl) phenyl]carbamate

Step 1: To a solution of sodium hydroxide (2.19 g, 54.8 mmol, 3.00 eq) in water (10.0 mL) and ethanol (10.0 mL) was added methyl 2,3-dimethylbenzoate (3.00 g, 18.3 mmol, 1.00 eq) in one portion. The reaction was stirred at 20° C. for 12 h. The mixture was concentrated under reduced pressure and poured into water (50.0 mL). The pH was adjusted to pH=1-2 with 36% aqueous hydrochloric acid, and the mixture was filtered. The filter cake was washed with water (2×20.0 mL) and dried under reduced pressure to afford 2,3-dimethylbenzoic acid.

Step 2: To a solution of 2,3-dimethylbenzoic acid (2.50 g, 16.6 mmol, 1.00 eq) in sulfuric acid (25.0 mL) was added potassium nitrate (2.02 g, 19.98 mmol, 1.20 eq) in portions at 0° C. The reaction was warmed to 15° C. and stirred for 12 h. The mixture was poured into ice-water (200 mL), filtered, and the filter cake was washed with water (2×50.0 mL). The filter cake was dried under reduced pressure to afford 2,3-dimethyl-5-nitro-benzoic acid.

Step 3: To a solution of 2,3-dimethyl-5-nitro-benzoic acid (4.50 g, 23.1 mmol, 1.00 eq) in tetrahydrofuran (100 mL) was added dimethyl sulfide borane (10.0 M, 4.61 mL, 2.00 eq) dropwise at 20° C. The reaction was heated to 60° C. and stirred for 5 h. The mixture was cooled to 0° C., then quenched with methanol (5.00 mL) and water (5.00 mL), and stirred at 20° C. for 0.5 h. The mixture was concentrated under reduced pressure and poured into saturated aqueous sodium bicarbonate (50.0 mL). The mixture was extracted with ethyl acetate (4×50.0 mL), and the combined organic layers were dried over anhydrous sodium sulfate, filtered, and concentrated under reduced pressure to give a residue. The residue was purified by silica gel column chromatography (petroleum ether/ethyl acetate=1/0 to 7/3) to afford (2,3-dimethyl-5-nitro-phenyl)methanol.

Step 4: To a solution of (2,3-dimethyl-5-nitro-phenyl)methanol (2.50 g, 13.8 mmol, 1.00 eq) in dichloromethane (20.0 mL) was added thionyl chloride (10.0 mL, 138 mmol, 10.0 eq) and N-methyl pyrrolidone (1.34 mL, 13.8 mmol, 1.00 eq) dropwise at 0° C. The reaction was stirred at 20° C. for 12 h. The mixture was concentrated under reduced pressure to give a residue. Acetonitrile (20.0 mL) was added, followed by triethylamine (5.76 mL, 41.4 mmol, 3.00 eq) and morpholine (1.46 mL, 16.5 mmol, 1.20 eq). The reaction was stirred at 20° C. for 12 h. The mixture was concentrated under reduced pressure to give a residue. The residue was purified by silica gel column chromatography (petroleum ether/ethyl acetate=1/0 to 0/1) to afford 4-[(2,3-dimethyl-5-nitro-phenyl)methyl]morpholine.

Step 5: To a solution of 4-[(2,3-dimethyl-5-nitro-phenyl)methyl]morpholine (2.50 g, 10.0 mmol, 1.00 eq) in ethanol (30.0 mL) and water (15.0 mL) were added ammonium chloride (534 mg, 10.0 mmol, 1 eq) and iron powder (2.79 g, 50.0 mmol, 5.00 eq) in portions at 20° C. The reaction was stirred at 90° C. for 12 h. The mixture was filtered, and the filter cake was washed with methanol (50.0 mL). The filtrate was concentrated under reduced pressure to give a residue. The residue was poured into saturated aqueous sodium bicarbonate (50.0 mL) and extracted with ethyl acetate (4×50.0 mL). The combined organic layers were dried over sodium sulfate, filtered, and the filtrate was concentrated under reduced pressure to afford 3,4-dimethyl-5-(morpholinomethyl)aniline.

Step 6: To a solution of 3,4-dimethyl-5-(morpholinomethyl)aniline (1.00 g, 4.54 mmol, 1.00 eq) and triethylamine (1.26 mL, 9.08 mmol, 2.00 eq) in tetrahydrofuran (10.0 mL) was added phenyl chloroformate (0.68 mL, 5.45 mmol, 1.20 eq) dropwise at 20° C. The reaction was stirred at 20° C. for 12 h. The mixture was concentrated and the obtained residue was purified by standard methods to afford phenyl N-[3,4-dimethyl-5-(morpholinomethyl)phenyl]carbamate.

Compound 119: General procedure A with variant iii) was used for the preparation from compound VI-A employing phenyl N-[3-chloro-5-(morpholinomethyl)phenyl]carbamate. ¹H NMR (400 MHz, DMSO-d₆) δ=10.98 (s, 1H), 8.92 (s, 1H), 8.15 (s, 1H), 7.69-7.53 (m, 4H), 7.19 (s, 1H), 6.92-6.81 (m, 2H), 5.11 (dd, J=5.2, 13.2 Hz, 1H), 4.48-4.27 (m, 4H), 3.60-3.53 (m, 4H), 3.39 (s, 2H), 2.98-2.82 (m, 1H), 2.64-2.56 (m, 1H), 2.45-2.37 (m, 1H), 2.34 (br s, 4H), 2.05-1.95 (m, 1H). MS (ESI) m/z 526.1 [M+H]⁺

### Preparation of phenyl N-[3-chloro-5-(morpholinomethyl)phenyl]carbamate

Step 1: To a solution of (3-chloro-5-nitro-phenyl)methanol (880 mg, 4.69 mmol, 1.00 eq) and N-methyl pyrrolidone (1.50 mL) in dichloromethane (10.0 mL) was added thionyl chloride (3.4 mL, 46.9 mmol, 10.0 eq) at 0° C. The reaction was stirred at 25° C. for 2 h. The mixture was concentrated under reduced pressure to give a residue. The residue was diluted with saturated aqueous sodium bicarbonate (60.0 mL) and extracted with ethyl acetate (4×50.0 mL). The combined organic layers were dried over sodium sulfate, filtered, and concentrated under reduced pressure to give a residue. The residue was purified by silica gel column chromatography (petroleum ether/ethyl acetate=3/1) to afford 1-chloro-3-(chloromethyl)-5-nitro-benzene.

Step 2: To a solution of 1-chloro-3-(chloromethyl)-5-nitro-benzene (1.40 g, 6.80 mmol, 1.00 eq) in acetonitrile (17.0 mL) were added triethylamine (2.36 mL, 17.0 mmol, 2.50 eq) and morpholine (0.78 mL, 8.83 mmol, 1.30 eq). The reaction was stirred at 25° C. for 12 h. The mixture was concentrated under reduced pressure to give a residue. The residue was purified by silica gel column chromatography (petroleum ether/ethyl acetate=7/1) to afford 4-[(3-chloro-5-nitro-phenyl)methyl]morpholine.

Step 3: To a solution of 4-[(3-chloro-5-nitro-phenyl)methyl]morpholine (980 mg, 3.82 mmol, 1.00 eq) in ethanol (16.0 mL) and water (8.00 mL) were added ammonium chloride (204 mg, 3.82 mmol, 1.00 eq) and iron powder (1.07 g, 19.1 mmol, 5.00 eq). The reaction was stirred at 90° C. for 12 h. The mixture was filtered and washed with ethyl acetate (20.0 mL), and the filtrate was extracted with ethyl acetate (3×20.0 mL). The combined organic layers were washed with brine (25.0 mL), dried over anhydrous sodium sulfate, filtered, and concentrated under reduced pressure to afford 3-chloro-5-(morpholinomethyl)aniline.

Step 4: To a solution of phenyl chloroformate (0.21 mL, 1.70 mmol, 1.10 eq) in tetrahydrofuran (20.0 mL) was added triethylamine (0.43 mL, 3.09 mmol, 2.00 eq) and 3-chloro-5-(morpholinomethyl)aniline (350 mg, 1.54 mmol, 1.00 eq). The mixture was stirred at 25° C. for 0.5 h. The mixture was filtered and concentrated and the obtained residue was purified by standard methods to afford phenyl N-[3-chloro-5-(morpholinomethyl)phenyl]carbamate.

### Compound 120:

Step 1: To a solution of 3-chloro-4-methyl-benzoic acid (20.0 g, 117 mmol, 1.00 eq) in sulfuric acid (80.0 mL) was added 1-iodopyrrolidine-2,5-dione (29.0 g, 129 mmol, 1.10 eq). The reaction was stirred at 25° C. for 1 h. The mixture was poured slowly into stirred ice water (300 mL). The resulting suspension was filtered, and the filter cake was washed with water (100 mL) and dried under reduced pressure. Methanol (200 mL) was added, and the mixture was concentrated under reduced pressure to afford 3-chloro-5-iodo-4-methyl-benzoic acid.

Step 2: To a solution of 3-chloro-5-iodo-4-methyl-benzoic acid (34.8 g, 117 mmol, 1.00 eq) in methanol (500 mL) was added thionyl chloride (27.9 g, 234 mmol, 17.0 mL, 2.00 eq) dropwise at 0° C. The reaction was stirred at 60° C. for 12 h. The mixture was concentrated to 100 mL under reduced pressure, and the resulting suspension was filtered. The filter cake was washed with methanol (30.0 mL) and dried under reduced pressure to afford methyl 3-chloro-5-iodo-4-methyl-benzoate.

Step 3: A solution of methyl 3-chloro-5-iodo-4-methyl-benzoate (10.0 g, 32.2 mmol, 1.00 eq), 4,4,5,5-tetramethyl-2-(4,4,5,5-tetramethyl-1,3,2-dioxaborolan-2-yl)-1,3,2-dioxaborolane (16.4 g, 64.4 mmol, 2.00 eq), potassium acetate (9.48 g, 96.6 mmol, 3.00 eq) and (1,1-bis(diphenylphosphino)ferrocene) dichloropalladium(II) (2.36 g, 3.22 mmol, 0.10 eq) in dioxane (200 mL) was stirred at 110° C. for 12 h. The mixture was concentrated under reduced pressure to give a residue. The residue was diluted with water (200 mL) and extracted with ethyl acetate (2×200 mL). The combined organic layers were washed with water (100 mL), dried over anhydrous sodium sulfate, filtered, and the filtrate was concentrated to afford a residue. The residue was purified by silica gel column chromatography (petroleum ether/ethyl acetate=1/0 to 100/5) to afford a residue. The residue was triturated with petroleum ether (100 mL), filtered, and the filter cake was washed with petroleum ether (50.0 mL) and dried under reduced pressure to afford methyl 3-chloro-4-methyl-5-(4,4,5,5-tetramethyl-1,3,2-dioxaborolan-2-yl)benzoate.

Step 4: To a solution of tert-butyl 2-bromoacetate (0.79 mL, 5.37 mmol, 1.00 eq), palladium acetate (36.1 mg, 161 μmol, 0.03 eq), potassium phosphate (5.70 g, 26.8 mmol, 5.00 eq) and tris-o-tolylphosphane (147 mg, 483 μmol, 0.09 eq) in tetrahydrofuran (40.0 mL) was added methyl 3-chloro-4-methyl-5-(4,4,5,5-tetramethyl-1,3,2-dioxaborolan-2-yl)benzoate (2.00 g, 6.44 mmol, 1.20 eq). The reaction was stirred at 25° C. for 12 h under nitrogen. The mixture was filtered, and the filtrate was concentrated under reduced pressure to afford a residue. The residue was purified by silica gel column chromatography (petroleum ether/ethyl acetate=1/0 to 5/1) to afford a residue. The residue was triturated with petroleum ether (10.0 mL), filtered, and the filtrate was concentrated to afford methyl 3-(2-tert-butoxy-2-oxo-ethyl)-5-chloro-4-methyl-benzoate.

Step 5: To a solution of methyl 3-(2-tert-butoxy-2-oxo-ethyl)-5-chloro-4-methyl-benzoate (400 mg, 1.34 mmol, 1.00 eq) in methanol (3.00 mL) was added a solution of sodium hydroxide (107 mg, 2.68 mmol, 2.00 eq) in water (3.00 mL). The reaction was stirred at 25° C. for 12 h. The mixture was concentrated under reduced pressure to give a residue. The residue was diluted with 1 M hydrochloric acid (3.00 mL) and extracted with ethyl acetate (50.0 mL). The organic layer was dried over anhydrous sodium sulfate, filtered, and the filtrate was concentrated to afford 3-(2-(tert-butoxy)-2-oxoethyl)-5-chloro-4-methylbenzoic acid.

Step 6: To a solution of 3-(2-tert-butoxy-2-oxo-ethyl)-5-chloro-4-methyl-benzoic acid (300 mg, 1.05 mmol, 1.00 eq) and triethylamine (0.16 mL, 1.16 mmol, 1.10 eq) in toluene (3.00 mL) was added diphenylphosphoryl azide (0.25 mL, 1.16 mmol, 1.10 eq). The reaction was stirred at 20° C. for 10 min, then phenol (0.46 mL, 5.27 mmol, 5.00 eq) was added. The reaction was stirred at 100° C. for 30 min. The mixture was concentrated under reduced pressure to give a residue. The residue was purified by silica gel column chromatography (petroleum ether/ethyl acetate=1/0 to 5/1) to afford tert-butyl 2-(3-chloro-2-methyl-5-((phenoxycarbonyl)amino)phenyl)acetate.

Step 7: To a solution of 3-(6-(aminomethyl)-1-oxoisoindolin-2-yl)piperidine-2,6-dione hydrochloride VI-A (150 mg, 484 μmol, 1.00 eq, hydrochloride) and triethylamine (135 μL, 969 μmol, 2.00 eq) in dimethylformamide (2.00 mL) was added tert-butyl 2-(3-chloro-2-methyl-5-((phenoxycarbonyl)amino)phenyl)acetate (182 mg, 484 μmol, 1.00 eq). The reaction was stirred at 25° C. for 12 h. The mixture was purified by reversed phase column chromatography and lyophilized to afford tert-butyl 2-(3-chloro-5-(3-((2-(2,6-dioxopiperidin-3-yl)-3-oxoisoindolin-5-yl)methyl)ureido)-2-methylphenyl)acetate.

Step 8: To a solution of tert-butyl 2-(3-chloro-5-(3-((2-(2,6-dioxopiperidin-3-yl)-3-oxoisoindolin-5-yl)methyl)ureido)-2-methylphenyl)acetate (240 mg, 432 μmol, 1.00 eq) in dichloromethane (5.00 mL) was added trifluoroacetic acid (5.00 mL). The reaction was stirred at 20° C. for 1 h. The mixture was concentrated and the obtained residue was purified by standard methods to afford Compound 120.

¹H NMR (400 MHz, CDCl₃) δ=12.42 (br s, 1H), 10.98 (s, 1H), 8.71 (s, 1H), 7.66 (s, 1H), 7.59 (d, J=2.0 Hz, 1H), 7.56 (s, 2H), 7.08 (d, J=1.8 Hz, 1H), 6.79 (br t, J=5.7 Hz, 1H), 5.11 (dd, J=5.3, 13.4 Hz, 1H), 4.48-4.27 (m, 4H), 3.60 (s, 2H), 2.97-2.84 (m, 1H), 2.62-2.58 (m, 1H), 2.41-2.37 (m, 1H), 2.17 (s, 3H), 2.04-1.93 (m, 1H). MS (ESI) m/z 499.1 [M+H]⁺

Compound 121: General procedure A with variant iii) was used for the preparation from compound VI-A employing phenyl (3-(pyridin-2-yloxy)phenyl)carbamate. ¹H NMR (400 MHz, DMSO-d₆) δ=11.00 (s, 1H), 8.80 (s, 1H), 8.17 (dd, J=4.9, 1.8 Hz, 1H), 7.79-7.89 (m, 1H), 7.66 (s, 1H), 7.56 (s, 2H), 7.34 (t, J=2.0 Hz, 1H), 7.20-7.29 (m, 1H), 7.10-7.16 (m, 2H), 7.00 (d, J=8.3 Hz, 1H), 6.75-6.82 (m, 1H), 6.65 (dd, J=8.0, 1.7 Hz, 1H), 5.12 (dd, J=13.3, 5.1 Hz, 1H), 4.28-4.47 (m, 4H), 2.85-2.98 (m, 1H), 2.60 (br d, J=16.5 Hz, 1H), 2.35-2.47 (m, 1H), 1.94-2.05 (m, 1H). MS (ESI) m/z 486.1 [M+H]⁺

Preparation of phenyl (3-(pyridin-2-yloxy)phenyl)carbamate: To a solution of 3-(pyridin-2-yloxy)aniline (0.250 g, 1.34 mmol, 1.00 eq) and triethylamine (0.56 mL, 4.03 mmol, 3.00 eq) in dichloromethane (5.00 mL) was added phenyl chloroformate (185 μL, 1.48 mmol, 1.10 eq) dropwise at 0° C. The reaction was stirred at 20° C. for 3 h. Water (5.00 mL) was added, and the organic layer was separated, dried over sodium sulfate, filtered, and concentrated to give phenyl (3-(pyridin-2-yloxy)phenyl)carbamate.

Compound 122: General procedure A with variant iii) was used for the preparation from compound VI-A employing phenyl (3-chloro-4-methyl-5-morpholinophenyl)carbamate. ¹H NMR (400 MHz, DMSO-d₆) δ=10.98 (br s, 1H), 8.76 (s, 1H), 7.65 (s, 1H), 7.56 (s, 2H), 7.37 (d, J=2.0 Hz, 1H), 7.00 (d, J=1.8 Hz, 1H), 6.80 (t, J=6.1 Hz, 1H), 5.11 (dd, J=5.1, 13.3 Hz, 1H), 4.48-4.35 (m, 3H), 4.34-4.25 (m, 1H), 3.76-3.66 (m, 4H), 2.98-2.84 (m, 1H), 2.82-2.71 (m, 4H), 2.68-2.55 (m, 1H), 2.39-2.31 (m, 1H), 2.19 (s, 3H), 2.06-1.91 (m, 1H). MS (ESI) m/z 526.3 [M+H]⁺

### Preparation of phenyl (3-chloro-4-methyl-5-morpholinophenyl)carbamate

Step 1: To a solution of methyl 3-chloro-5-iodo-4-methylbenzoate (4.00 g, 12.9 mmol, 1.00 eq) in toluene (40.0 mL) under nitrogen was added morpholine (1.36 mL, 15.5 mmol, 1.20 eq) and cesium carbonate (21.0 g, 64.4 mmol, 5.00 eq). A separate solution of palladium acetate (289 mg, 1.29 mmol, 0.10 eq) and 2,2′-bis(diphenylphosphino)-1,1′-binaphthalene (802 mg, 1.29 mmol, 0.10 eq) in toluene (20.0 mL) was added. The reaction was stirred at 120° C. for 12 hours. The mixture was filtered and concentrated under reduced pressure to give a residue. The residue was purified by silica gel column chromatography (petroleum ether/ethyl acetate=1/0 to 10/1) to afford methyl 3-chloro-4-methyl-5-morpholinobenzoate.

Step 2: To a solution of methyl 3-chloro-4-methyl-5-morpholinobenzoate (1.55 g, 5.75 mmol, 1.00 eq) in tetrahydrofuran (15.0 mL) and water (5.00 mL) was added lithium hydroxide (275 mg, 11.5 mmol, 2.00 eq). The reaction was stirred at 20° C. for 12 h. Water (20.0 mL) was added, and the mixture was extracted with ethyl acetate (2×25.0 mL). The pH of the aqueous layer was adjusted to pH=7 by addition of 1M hydrochloric acid, and it was extracted with ethyl acetate (2×30.0 mL). The combined organic layers were dried over anhydrous sodium sulfate, filtered, and concentrated under reduced pressure to give 3-chloro-4-methyl-5-morpholinobenzoic acid.

Step 3: To a solution of 3-chloro-4-methyl-5-morpholinobenzoic acid (600 mg, 2.35 mmol, 1.00 eq) and triethylamine (0.36 mL, 2.58 mmol, 1.10 eq) in toluene (6.00 mL) was added diphenylphosphoryl azide (0.56 mL, 2.58 mmol, 1.10 eq). The reaction was stirred at 20° C. for 10 min. Then phenol (1.03 mL, 11.7 mmol, 5.00 eq) was added, and the reaction was stirred at 100° C. for 30 min. The mixture was concentrated and the obtained residue was purified by standard methods to give phenyl (3-chloro-4-methyl-5-morpholinophenyl)carbamate.

Compound 123: General procedure A with variant iii) was used for the preparation from compound VI-A employing phenyl pyridin-2-ylcarbamate. ¹H NMR (400 MHz, DMSO-d₆) δ=11.20 (s, 1H), 10.97 (s, 1H), 8.30 (s, 1H), 8.23 (dd, J=1.2, 5.6 Hz, 1H), 8.09 (t, J=8.0 Hz, 1H), 7.69 (s, 1H), 7.62-7.55 (m, 2H), 7.41 (d, J=8.8 Hz, 1H), 7.22 (t, J=6.4 Hz, 1H), 5.10 (dd, J=4.8 Hz, 13.2 Hz, 1H), 4.50 (m, J=5.6 Hz, 2H), 4.37 (dd, J=17.2, 54.0 Hz, 2H), 2.90 (ddd, J=5.2, 13.6, 17.6 Hz, 1H), 2.63-2.55 (m, 1H), 2.45-2.33 (m, 1H), 2.03-1.94 (m, 1H). LCMS m/z 394.0 [M+H]⁺

Preparation of phenyl pyridin-2-ylcarbamate: To a solution of pyridin-2-amine (2.00 g, 21.3 mmol, 1.00 eq) in tetrahydrofuran (50.0 mL) was added phenyl chloroformate (3.66 g, 23.4 mmol, 1.10 eq) and triethylamine (4.30 g, 42.5 mmol, 2.00 eq). The reaction was stirred at 25° C. for 0.5 h. The mixture was concentrated and the obtained residue was purified by standard methods to afford phenyl pyridin-2-ylcarbamate.

Compound 124: General procedure A with variant iii) was used for the preparation from compound VI-A employing phenyl (3-chloro-4-methyl-5-((4-methylpiperazin-1-yl)methyl)phenyl)carbamate. ¹H NMR (400 MHz, DMSO-d₆) δ=10.99 (s, 1H), 8.80 (br s, 1H), 8.25-8.14 (m, 1H), 7.74-7.62 (m, 2H), 7.56 (s, 2H), 7.11 (d, J=1.8 Hz, 1H), 6.83 (br s, 1H), 5.12 (dd, J=5.0, 13.2 Hz, 1H), 4.55-4.23 (m, 4H), 3.38 (s, 2H), 3.00-2.85 (m, 1H), 2.69-2.56 (m, 2H), 2.47-2.29 (m, 8H), 2.25 (s, 3H), 2.19 (s, 3H), 2.07-1.94 (m, 1H). MS (ESI) m/z 553.3 [M+H]⁺

### Preparation of phenyl (3-chloro-4-methyl-5-((4-methylpiperazin-1-yl)methyl)phenyl)carbamate

Step 1: To a solution of (3-chloro-2-methyl-5-nitrophenyl)methanol (2.00 g, 9.92 mmol, 1.00 eq) in dichloromethane (20.0 mL) was added thionyl chloride (5.90 g, 49.6 mmol, 3.60 mL, 5.00 eq) dropwise. The reaction was stirred at 25° C. for 2 h. The mixture was concentrated under reduced pressure to afford 1-chloro-3-(chloromethyl)-2-methyl-5-nitrobenzene (2.20 g, 10.0 mmol, crude) as a gray solid. To a solution of 1-chloro-3-(chloromethyl)-2-methyl-5-nitrobenzene (2.20 g, 10.00 mmol, 1.00 eq) and triethylamine (3.48 mL, 25.0 mmol, 2.50 eq) in acetonitrile (20.0 mL) was added 1-methylpiperazine (1.44 mL, 13.0 mmol, 1.30 eq). The reaction was stirred at 25° C. for 10 h. The mixture was diluted with water (6.00 mL) and extracted with ethyl acetate (3×25.0 mL). The combined organic layers were washed with brine (10.0 mL), dried over sodium sulfate, filtered, and concentrated under reduced pressure to give a residue. The residue was purified by silica gel column chromatography (petroleum ether/ethyl acetate=1/0 to 1/1) to afford 1-(3-chloro-2-methyl-5-nitrobenzyl)-4-methylpiperazine.

Step 2: A mixture of 1-(3-chloro-2-methyl-5-nitrobenzyl)-4-methylpiperazine (0.70 g, 2.47 mmol, 1.00 eq), ammonium chloride (132 mg, 2.47 mmol, 1.00 eq) and ferrous powder (689 mg, 12.3 mmol, 5.00 eq) in ethanol (10.0 mL) and water (5.00 mL) was stirred at 90° C. for 10 h. The reaction was filtered and concentrated under reduced pressure to give a residue.

The residue was purified was by reversed phase column chromatography and lyophilized to afford 3-chloro-4-methyl-5-((4-methylpiperazin-1-yl)methyl)aniline.

Step 3: To a solution of 3-chloro-4-methyl-5-((4-methylpiperazin-1-yl)methyl)aniline (0.30 g, 1.18 mmol, 1.00 eq) and potassium carbonate (326 mg, 2.36 mmol, 2.00 eq) in acetone (5.00 mL) was added phenyl chloroformate (0.22 mL, 1.77 mmol, 1.50 eq) dropwise at 0° C. The reaction was stirred at 25° C. for 3 h. The mixture was concentrated and the obtained residue was purified by standard methods to afford phenyl (3-chloro-4-methyl-5-((4-methylpiperazin-1-yl)methyl)phenyl) carbamate.

Compound 125: General procedure A with variant iii) was used for the preparation from compound VI-A employing phenyl (3-chloro-5-(2-methoxyethoxy)-4-methylphenyl)carbamate. ¹H NMR (400 MHz, DMSO-d₆) δ=10.98 (br s, 1H), 8.80 (s, 1H), 7.66 (s, 1H), 7.57 (s, 2H), 7.19 (d, J=1.7 Hz, 1H), 7.01 (d, J=1.6 Hz, 1H), 6.88 (br t, J=6.0 Hz, 1H), 5.12 (dd, J=5.0, 13.3 Hz, 1H), 4.50-4.26 (m, 4H), 4.12-3.98 (m, 2H), 3.75-3.61 (m, 2H), 3.33 (br s, 3H), 3.01-2.83 (m, 1H), 2.68-2.59 (m, 1H), 2.44-2.38 (m, 1H), 2.11 (s, 3H), 2.06-1.96 (m, 1H). MS (ESI) m/z 515.1 [M+H]⁺

### Preparation of phenyl (3-chloro-5-(2-methoxyethoxy)-4-methylphenyl)carbamate

Step 1: A mixture of methyl 3-chloro-5-hydroxy-4-methyl-benzoate (1.00 g, 4.98 mmol, 1.00 eq), 1-bromo-2-methoxy-ethane (0.94 mL, 9.97 mmol, 2.00 eq) and potassium carbonate (2.76 g, 19.9 mmol, 4.00 eq) in acetonitrile (20.0 mL) was stirred at 85° C. for 12 h. The mixture was filtered, and the filtrate was concentrated under reduced pressure to give a residue. The residue was purified by silica gel column chromatography (petroleum ether/ethyl acetate=20/1 to 5/1) to give methyl 3-chloro-5-(2-methoxyethoxy)-4-methyl-benzoate.

Step 2: A mixture of methyl 3-chloro-5-(2-methoxyethoxy)-4-methyl-benzoate (1.00 g, 3.87 mmol, 1.00 eq) and sodium hydroxide (309 mg, 7.73 mmol, 2.00 eq) in methanol (10.0 mL) and water (10.0 mL) was stirred at 70° C. for 12 h. The mixture was concentrated under reduced pressure to give a residue. The residue was diluted with water (10.0 mL), and the pH was adjusted to pH=3 with hydrogen chloride (1N). The mixture was extracted with ethyl acetate (3×10.0 mL). The combined organic layers were washed with brine (3×10.0 mL), dried with anhydrous sodium sulfate, filtered, and concentrated under reduced pressure to afford 3-chloro-5-(2-methoxyethoxy)-4-methyl-benzoic acid.

Step 3: A solution of 3-chloro-5-(2-methoxyethoxy)-4-methyl-benzoic acid (650 mg, 2.66 mmol, 1.00 eq), diphenyl phosphoryl azide (0.86 mL, 3.98 mmol, 1.50 eq), and triethylamine (0.74 mL, 5.31 mmol, 2.00 eq) in 2-methylpropan-2-ol (10.0 mL) was stirred at 100° C. for 12 h. The mixture was diluted with water (50.0 mL) and extracted with ethyl acetate (3×20.0 mL). The combined organic layers were washed with brine (3×10.0 mL), dried with anhydrous sodium sulfate, filtered, and concentrated under reduced pressure to give a residue. The residue was purified by silica gel column chromatography (petroleum ether/ethyl acetate=10/1 to 3/1) to give tert-butyl (3-chloro-5-(2-methoxyethoxy)-4-methylphenyl)carbamate.

Step 4: To a solution of tert-butyl (3-chloro-5-(2-methoxyethoxy)-4-methylphenyl)carbamate (650 mg, 2.06 mmol, 1.00 eq) in hydrogen chloride/ethyl acetate (2.00 mL) was stirred at 25° C. for 1 h. The mixture was concentrated under reduced pressure to afford 3-chloro-5-(2-methoxyethoxy)-4-methyl-aniline hydrochloride.

Step 5: To a solution of 3-chloro-5-(2-methoxyethoxy)-4-methyl-aniline hydrochloride (430 mg, 1.99 mmol, 1.00 eq, hydrochloride) and triethylamine (555 μL, 3.99 mmol, 2.00 eq) in dichloromethane (10.0 mL) was added phenyl chloroformate (0.30 mL, 2.39 mmol, 1.20 eq) dropwise at 0° C. The reaction was stirred at 25° C. for 0.5 h. The mixture was concentrated and the obtained residue was purified by standard methods to afford phenyl (3-chloro-5-(2-methoxyethoxy)-4-methylphenyl)carbamate.

Compound 126: General procedure A with variant iii) was used for the preparation from compound VI-A employing phenyl (3-chloro-4-methyl-5-((4-morpholinopiperidin-1-yl)methyl)phenyl)carbamate. ¹H NMR (400 MHz, DMSO-d₆) δ=10.99 (s, 1H), 8.83 (s, 1H), 8.18 (s, 1H), 7.69-7.61 (m, 2H), 7.56 (d, J=0.8 Hz, 2H), 7.12 (d, J=2.0 Hz, 1H), 6.88-6.80 (m, 1H), 5.12 (dd, J=5.2, 13.2 Hz, 1H), 4.46-4.25 (m, 4H), 3.64-3.48 (m, 4H), 3.37 (s, 2H), 2.98-2.77 (m, 3H), 2.62-2.57 (m, 1H), 2.46 (br d, J=3.8 Hz, 4H), 2.40 (br dd, J=4.4, 13.2 Hz, 1H), 2.24 (s, 3H), 2.15 (br t, J=10.8 Hz, 1H), 2.05-1.89 (m, 3H), 1.74 (br d, J=11.2 Hz, 2H), 1.45-1.29 (m, 2H). MS (ESI) m/z 623.1 [M+H]⁺

### Preparation of phenyl (3-chloro-4-methyl-5-((4-morpholinopiperidin-1-yl)methyl)phenyl)carbamate

Step 1: To a solution of (3-chloro-2-methyl-5-nitrophenyl)methanol (1.00 g, 4.96 mmol, 1.00 eq) in dichloromethane (15.0 mL) was added thionyl chloride (2.95 g, 24.8 mmol, 1.80 mL, 5.00 eq) at 0° C. Then the reaction was stirred at 25° C. for 2 h. The mixture was concentrated under reduced pressure to give 1-chloro-3-(chloromethyl)-2-methyl-5-nitrobenzene (1.10 g, 4.96 mmol, crude).

Step 2: To a solution of 1-chloro-3-(chloromethyl)-2-methyl-5-nitrobenzene (1.10 g, 5.00 mmol, 1.00 eq) and triethylamine (1.26 g, 12.5 mmol, 1.74 mL, 2.50 eq) in acetonitrile (10.0 mL) was added 4-(piperidin-4-yl)morpholine (1.06 g, 6.25 mmol, 1.25 eq). The reaction was stirred at 25° C. for 10 h. The mixture was concentrated under reduced pressure to give a residue. The residue was purified was by reversed phase column chromatography and lyophilized to afford 4-(1-(3-chloro-2-methyl-5-nitrobenzyl)piperidin-4-yl)morpholine.

Step 3: A mixture of 4-(1-(3-chloro-2-methyl-5-nitrobenzyl)piperidin-4-yl)morpholine (1.00 g, 2.83 mmol, 1.00 eq), ammonium chloride (151 mg, 2.83 mmol, 1.00 eq) and ferrous powder (789 mg, 14.1 mmol, 5.00 eq) in ethanol (20.0 mL) and water (10.0 mL) was stirred at 90° C. for 10 h. The mixture was filtered and concentrated under reduced pressure to afford 3-chloro-4-methyl-5-((4-morpholinopiperidin-1-yl)methyl)aniline.

Step 4: To a solution of 3-chloro-4-methyl-5-((4-morpholinopiperidin-1-yl)methyl)aniline (0.92 g, 2.84 mmol, 1.00 eq) and potassium carbonate (785 mg, 5.68 mmol, 2.00 eq) in acetone (10.0 mL) was added phenyl chloroformate (0.53 mL, 4.26 mmol, 1.50 eq) dropwise. The reaction was stirred at 25° C. for 10 h. The mixture was diluted with water (10.0 mL) and extracted with ethyl acetate (3×35.0 mL). The combined organic layers were washed, dried, filtered, and concentrated to afford phenyl (3-chloro-4-methyl-5-((4-morpholinopiperidin-1-yl) methyl)phenyl)carbamate.

Compound 127: General procedure A with variant iii) was used for the preparation from compound VI-A employing phenyl (3-chloro-4-methyl-5-(2-morpholinoethoxy) phenyl)carbamate. ¹H NMR (400 MHz, DMSO-d₆) δ=11.0 (s, 1H), 10.8-10.6 (m, 1H), 9.03 (s, 1H), 7.66 (s, 1H), 7.57 (s, 2H), 7.20-7.11 (m, 2H), 6.99 (br t, J=6.0 Hz, 1H), 5.11 (dd, J=5.1, 13.3 Hz, 1H), 4.49-4.22 (m, 6H), 3.99 (br d, J=12.5 Hz, 2H), 3.76 (br t, J=11.9 Hz, 2H), 3.60 (br d, J=1.1 Hz, 2H), 3.50 (br d, J=12.6 Hz, 2H), 3.27-3.15 (m, 2H), 3.00-2.85 (m, 1H), 2.71-2.58 (m, 1H), 2.40-2.30 (m, 1H), 2.16 (s, 3H), 2.03-1.94 (m, 1H). MS (ESI) m/z 570.3 [M+H]⁺

### Preparation of phenyl (3-chloro-4-methyl-5-(2-morpholinoethoxy)phenyl)carbamate

Step 1: To a solution of methyl 3-chloro-5-hydroxy-4-methylbenzoate (600 mg, 3.00 mmol, 1.00 eq) in acetonitrile (6.00 mL) was added potassium iodide (49.7 mg, 0.30 mmol, 0.10 eq), potassium carbonate (1.65 g, 12.0 mmol, 4.00 eq) and 4-(2-chloroethyl)morpholine hydrochloric acid (1.11 g, 5.98 mmol, 2.00 eq, hydrochloric acid). The reaction was stirred at 80° C. for 12 h. The mixture was filtered and concentrated under reduced pressure to give a residue. The residue was purified by silica gel chromatography (dichloromethane/methanol=1/0 to 10/1) to afford methyl 3-chloro-4-methyl-5-(2-morpholinoethoxy)benzoate.

Step 2: To a solution of methyl 3-chloro-4-methyl-5-(2-morpholinoethoxy)benzoate (770 mg, 2.45 mmol, 1.00 eq) in tetrahydrofuran (3.00 mL) and water (1.00 mL) was added lithium hydroxide monohydrate (309 mg, 7.36 mmol, 3.00 eq). The reaction was stirred at 20° C. for 12 h. The mixture was concentrated under reduced pressure to give a residue. The residue was purified by reversed phase column chromatography and lyophilized to give a residue. Water (20 mL) and hydrochloric acid (0.20 mL) were added to the residue, and it was lyophilized to give 3-chloro-4-methyl-5-(2-morpholinoethoxy)benzoic acid

Step 3: To a solution of 3-chloro-4-methyl-5-(2-morpholinoethoxy)benzoic acid (600 mg, 2.00 mmol, 1.00 eq) in toluene (6.00 mL) was added triethylamine (446 mg, 4.40 mmol, 2.20 eq) and diphenylphosphoryl azide (606 mg, 2.20 mmol, 1.10 eq). The reaction was stirred at 20° C. for 10 min, then phenol (942 mg, 10.0 mmol, 5.00 eq) was added. The reaction was stirred at 100° C. for 30 min. The mixture was concentrated and the obtained residue was purified by standard methods to give phenyl (3-chloro-4-methyl-5-(2-morpholinoethoxy)phenyl)carbamate.

Compound 128: General procedure A with variant iii) was used for the preparation from compound VI-A employing phenyl (1-methyl-1H-pyrrol-3-yl)carbamate. ¹H NMR (400 MHz, DMSO-d₆) δ=10.97 (s, 1H), 7.99 (s, 1H), 7.63 (s, 1H), 7.53 (s, 2H), 6.71 (t, J=2.0 Hz, 1H), 6.46 (t, J=2.5 Hz, 1H), 6.42 (br t, J=5.9 Hz, 1H), 5.80-5.75 (m, 1H), 5.11 (dd, J=5.1, 13.3 Hz, 1H), 4.47-4.25 (m, 4H), 3.51 (s, 3H), 2.97-2.84 (m, 1H), 2.63-2.55 (m, 1H), 2.39 (dd, J=4.4, 13.1 Hz, 1H), 2.03-1.95 (m, 1H). MS (ESI) m/z 396.1 [M+H]⁺

Preparation of phenyl (1-methyl-1H-pyrrol-3-yl)carbamate: To a solution of 1-methyl-1H-pyrrole-3-carboxylic acid (700 mg, 5.59 mmol, 1.00 eq) in toluene (7.00 mL) was added diphenylphosphoryl azide (1.33 mL, 6.15 mmol, 1.10 eq) and triethylamine (0.86 mL, 6.15 mmol, 1.10 eq). The reaction was stirred at 20° C. for 10 min, then phenol (2.46 mL, 28.0 mmol, 5.00 eq) was added and the reaction was stirred at 100° C. for 30 min. The mixture was concentrated and the obtained residue was purified by standard methods to give phenyl (1-methyl-1H-pyrrol-3-yl)carbamate.

Compound 129: General procedure A with variant iii) was used for the preparation from compound VI-A employing phenyl (3-chloro-5-(dimethylamino)-4-methylphenyl)carbamate. ¹H NMR (400 MHz, DMSO-d₆) δ=10.97 (br s, 1H), 8.78 (br s, 1H), 7.65 (s, 1H), 7.56 (s, 2H), 7.32 (d, J=2.0 Hz, 1H), 7.00 (d, J=2.0 Hz, 1H), 6.82 (br d, J=2.9 Hz, 1H), 5.11 (dd, J=5.1, 13.3 Hz, 1H), 4.47-4.27 (m, 4H), 2.97-2.85 (m, 1H), 2.64-2.60 (m, 1H), 2.59 (s, 6H), 2.39 (dd, J=4.4, 13.0 Hz, 1H), 2.19 (s, 3H), 2.04-1.96 (m, 1H). MS (ESI) m/z 484.2 [M+H]⁺

### Preparation of phenyl (3-chloro-5-(dimethylamino)-4-methylphenyl)carbamate

Step 1: To a solution of methyl 3-chloro-5-iodo-4-methylbenzoate (9.00 g, 29.0 mmol, 1.00 eq) in toluene (180 mL) was added 2,2′-bis(diphenylphosphino)-1,1′-binaphthalene (5.41 g, 8.70 mmol, 0.30 eq), diphenylmethanimine (5.84 mL, 34.8 mmol, 1.20 eq), sodium tert-butoxide (3.90 g, 40.6 mmol, 1.40 eq) and tris(dibenzylideneacetone)dipalladium (2.65 g, 2.90 mmol, 0.10 eq). The reaction was stirred at 80° C. for 12 h under nitrogen. The mixture was filtered and concentrated under reduced pressure to give a residue. The residue was purified by silica gel column chromatography (petroleum ether/ethyl acetate=1/0 to 10/1) to give methyl 3-chloro-5-((diphenylmethylene)amino)-4-methylbenzoate.

Step 2: To a solution of methyl 3-chloro-5-((diphenylmethylene)amino)-4-methylbenzoate (6.50 g, 17.9 mmol, 1.00 eq) in tetrahydrofuran (65.0 mL) was added hydrochloric acid (1.00 M, 17.9 mL, 1.00 eq). The reaction was stirred at 20° C. for 2 h. Water (60.0 mL) was added, followed by saturated sodium bicarbonate until pH=8: The mixture was extracted with ethyl acetate (3×60.0 mL). The organic phases were gathered, dried over sodium sulfate, filtered, and concentrated under reduced pressure to give a residue. The residue was purified by silica gel column chromatography (petroleum ether/ethyl acetate=1/0 to 5/1) to give methyl 3-amino-5-chloro-4-methylbenzoate

Step 3: To a solution of methyl 3-amino-5-chloro-4-methylbenzoate (2.86 g, 14.3 mmol, 1.00 eq) in dimethylformamide (30.0 mL) was added iodomethane (2.68 mL, 43.0 mmol, 3.00 eq) and potassium carbonate (7.92 g, 57.3 mmol, 4.00 eq). The reaction was stirred at 80° C. for 5 h. Water (40.0 mL) was added, and the mixture was extracted with ethyl acetate (3×50.0 mL). The organic layers were gathered, dried over sodium sulfate, filtered, and concentrated under reduced pressure to give a residue. The residue was purified by silica gel column chromatography (petroleum ether/ethyl acetate=1/0 to 5/1) to give methyl 3-chloro-5-(dimethylamino)-4-methylbenzoate.

Step 4: To a solution of methyl 3-chloro-5-(dimethylamino)-4-methylbenzoate (2.30 g, 10.1 mmol, 1.00 eq) in tetrahydrofuran (18.0 mL) and water (6.00 mL) was added lithium hydroxide (484 mg, 20.2 mmol, 2.00 eq). The reaction was stirred at 20° C. for 12 h. Water (20.0 mL) was added, and the mixture was extracted with ethyl acetate (2×40.0 mL). 1M hydrochloric acid was added to the aqueous layer until pH=7, and it was extracted with ethyl acetate (2×40.0 mL). The organic layers were gathered, dried over sodium sulfate, filtered, and concentrated under reduced pressure to afford 3-chloro-5-(dimethylamino)-4-methylbenzoic acid.

Step 5: To a solution of 3-chloro-5-(dimethylamino)-4-methylbenzoic acid (700 mg, 3.28 mmol, 1.00 eq) and triethylamine (0.50 mL, 3.60 mmol, 1.10 eq) in toluene (7.00 mL) was added diphenylphosphoryl azide (0.78 mL, 3.60 mmol, 1.10 eq). The reaction was stirred at 20° C. for 10 min, then phenol (1.44 mL, 16.4 mmol, 5.00 eq) was added. The reaction was stirred at 100° C. for 30 min. The mixture was concentrated and the obtained residue was purified by standard methods to give phenyl (3-chloro-5-(dimethylamino)-4-methylphenyl)carbamate.

Compound 130: General procedure A with variant iii) was used for the preparation from compound VI-A employing phenyl N-[3-[(1-methyl-4-piperidyl)oxy]phenyl]carbamate. ¹H NMR (DMSO-d₆) δ=10.96 (s, 1H), 8.61 (br s, 1H), 8.16 (s, 1H), 7.65 (s, 1H), 7.55 (s, 2H), 7.17 (s, 1H), 7.12-7.05 (m, 1H), 6.84 (d, J=8.4 Hz, 1H), 6.75 (br s, 1H), 6.48 (dd, J=8.2, 2.2 Hz, 1H), 5.11 (dd, J=13.3, 5.2 Hz, 1H), 4.46-4.37 (m, 3H), 4.30 (br d, J=17.0 Hz, 2H), 2.96-2.85 (m, 1H), 2.67-2.55 (m, 3H), 2.39 (br dd, J=13.1, 4.5 Hz, 1H), 2.30-2.23 (m, 2H), 2.27-2.23 (m, 2H), 2.21 (br s, 3H), 2.03-1.95 (m, 1H), 1.90 (br s, 2H), 1.63 (br d, J=8.2 Hz, 2H). MS (ESI) m/z 506.4[M+H]⁺

### Preparation of phenyl N-[3-[(1-methyl-4-piperidyl)oxy]phenyl]carbamate

Step 1: A solution of tert-butyl 4-(3-nitrophenoxy)piperidine-1-carboxylate (800 mg, 2.48 mmol, 1.00 eq) in hydrochloric acid/ethyl acetate (10.0 mL) was stirred at 25° C. for 0.5 h. The mixture was concentrated under reduced pressure to afford 4-(3-nitrophenoxy)piperidine.

Step 2: To a solution of 4-(3-nitrophenoxy)piperidine (500 mg, 2.25 mmol, 1.00 eq) in methanol (20.0 mL) was added formaldehyde (37% purity, 0.50 mL, 6.75 mmol, 3.00 eq) and sodium cyanoborohydride (424 mg, 6.75 mmol, 3.00 eq). The reaction was stirred at 25° C. for 4 h. The mixture was concentrated under reduced pressure to give a residue. Water (10.0 mL) was added and the solution was extracted with dichloromethane (3×20.0 mL). The combined organic layers were washed with brine (3×10.0 mL), dried over anhydrous sodium sulfate, filtered, and concentrated under reduced pressure to give a residue. The residue was purified by preparative TLC (dichloromethane/methanol=10/1) to afford 1-methyl-4-(3-nitrophenoxy) piperidine.

Step 3: To a solution of 1-methyl-4-(3-nitrophenoxy)piperidine (320 mg, 1.35 mmol, 1.00 eq) in tetrahydrofuran (5.00 mL) was added Pd/C 10% weight on C (100 mg, 1.00 eq). The reaction was stirred under hydrogen atmosphere (15 psi) at 20° C. for 4 h. The mixture was filtered, and the filtrate was concentrated under reduced pressure to afford 3-[(1-methyl-4-piperidyl)oxy]aniline.

Step 4: To a solution of 3-[(1-methyl-4-piperidyl)oxy]aniline (270 mg, 1.31 mmol, 1.00 eq) in dichloromethane (10.0 mL) was added pyridine (0.32 mL, 3.93 mmol, 3.00 eq) at 0° C., then phenyl chloroformate (0.18 mL, 1.44 mmol, 1.10 eq) was added. The reaction was stirred at 20° C. for 6 h. The mixture was concentrated under reduced pressure to give a residue. The residue was diluted with water (15.0 mL) and extracted with dichloromethane (3×20.0 mL). The combined organic layers were washed with brine (3×10.0 mL), dried over anhydrous sodium sulfate, filtered, and concentrated and the obtained residue was purified by standard methods to afford phenyl N-[3-[(1-methyl-4-piperidyl)oxy]phenyl]carbamate.

Compound 131: General procedure A with variant iii) was used for the preparation from compound VI-A employing phenyl (4-(2-hydroxypropan-2-yl)phenyl)carbamate. ¹H NMR (400 MHz, DMSO-d₆) δ=10.97 (br s, 1H), 8.56 (s, 1H), 7.67 (s, 1H), 7.57 (s, 2H), 7.31 (s, 4H), 6.73 (br t, J=6.0 Hz, 1H), 5.12 (dd, J=5.0, 13.2 Hz, 1H), 4.85 (s, 1H), 4.50-4.23 (m, 4H), 3.02-2.84 (m, 1H), 2.66-2.56 (m, 1H), 2.44-2.34 (m, 1H), 2.05-1.96 (m, 1H), 1.39 (s, 6H). MS (ESI) m/z 449.5 [M−H]⁻

### Preparation of phenyl (4-(2-hydroxypropan-2-yl)phenyl)carbamate

Step 1: To a mixture of 1-(4-nitrophenyl)ethanone (2.00 g, 12.1 mmol, 1.00 eq) in ethanol (18.0 mL) and water (9.00 mL) was added ammonium chloride (648 mg, 12.1 mmol, 1.00 eq) and ferrous powder (3.38 g, 60.6 mmol, 5.00 eq). The reaction was stirred at 90° C. for 10 h. The mixture was filtered, and the filtrate was extracted with ethyl acetate (3×30.0 mL). The combined organic layers were washed with brine (10.0 mL), dried over sodium sulfate, filtered, and concentrated under reduced pressure to afford 1-(4-aminophenyl)ethanone.

Step 2: To a solution of 1-(4-aminophenyl)ethanone (1.60 g, 11.8 mmol, 1.00 eq) in tetrahydrofuran (20.0 mL) was added methylmagnesium bromide (3.00 M, 11.8 mL, 3.00 eq) dropwise at 0° C. Then the reaction was stirred at 25° C. for 10 h. The mixture was quenched by addition saturated ammonium chloride (15.0 mL), diluted with water (10.0 mL), and extracted with ethyl acetate (3×55.0 mL). The combined organic layers were washed with brine (25.0 mL), dried over sodium sulfate, filtered, and concentrated under reduced pressure to give a residue. The residue was purified by preparative reversed phase preparative HPLC to afford 2-(4-aminophenyl)propan-2-ol.

Step 3: To a solution of 2-(4-aminophenyl)propan-2-ol (0.45 g, 2.98 mmol, 1.00 eq) and 2,6-dimethylpyridine (0.38 mL, 3.27 mmol, 1.10 eq) in tetrahydrofuran (3.00 mL) and trichloromethane (3.00 mL) was added phenyl chloroformate (0.37 mL, 2.98 mmol, 1.00 eq) at 0° C. The reaction was stirred at 0° C. for 1 h. The mixture was diluted with water (10.0 mL) and extracted with dichloromethane (3×35.0 mL). The combined organic layers were washed with brine (10.0 mL), dried over sodium sulfate, filtered and concentrated under reduced pressure to afford phenyl (4-(2-hydroxypropan-2-yl)phenyl)carbamate.

Compound 132: General procedure A with variant iii) was used for the preparation from compound VI-A employing phenyl (3-((1,3-dioxan-5-yl)methyl)-5-chloro-4-methylphenyl)carbamate. ¹H NMR (400 MHz, DMSO-d₆) δ=10.98 (s, 1H), 8.77 (s, 1H), 7.66 (s, 1H), 7.57 (s, 3H), 7.02 (d, J=2.0 Hz, 1H), 6.86 (t, J=6.0 Hz, 1H), 5.12 (dd, J=5.2, 13.2 Hz, 1H), 4.83 (d, J=6.0 Hz, 1H), 4.69 (d, J=6.0 Hz, 1H), 4.49-4.26 (m, 4H), 3.86 (dd, J=3.8, 11.2 Hz, 2H), 3.51 (dd, J=7.6, 11.2 Hz, 2H), 2.99-2.83 (m, 1H), 2.66-2.54 (m, 3H), 2.40 (dq, J=4.4, 13.2 Hz, 1H), 2.22 (s, 3H), 2.05-1.97 (m, 1H), 1.93 (dt, J=3.6, 7.4 Hz, 1H). MS (ESI) m/z 541.3 [M+H]⁺

### Preparation of phenyl (3-((1,3-dioxan-5-yl)methyl)-5-chloro-4-methylphenyl)carbamate

Step 1: To a solution of 3-chloro-2-methylbenzoic acid (41.0 g, 240 mmol, 1.00 eq) in sulfuric acid (200 mL) was added nitric acid (12.3 mL, 264 mmol, 1.10 eq) dropwise at −10° C. The reaction was stirred at −10° C. for 1 h. The mixture was poured into stirred ice water (200 mL). The resulting precipitate was collected by filtration and washed with water to afford 3-chloro-2-methyl-5-nitrobenzoic acid.

Step 2: To a solution of 3-chloro-2-methyl-5-nitrobenzoic acid (52.0 g, 67.5 mmol, 1.00 eq) in tetrahydrofuran (400 mL) was added borane dimethyl sulfide complex (10.0 M, 13.5 mL, 2.00 eq) at 0° C. The reaction was stirred at 25° C. for 10 h. Water (25.0 mL) was added at 0° C., and the pH was adjusted to pH=10.0 by addition of 15% sodium hydroxide solution. The mixture was extracted with ethyl acetate (3×300 mL). The combined organic layers were washed with brine (2×100 mL), dried over sodium sulfate, filtered, and concentrated under reduced pressure to give a residue. The residue was purified by silica gel column chromatography (petroleum ether/ethyl acetate=3/1) to afford (3-chloro-2-methyl-5-nitrophenyl)methanol.

Step 3: To a solution of (3-chloro-2-methyl-5-nitrophenyl)methanol (1.10 g, 5.46 mmol, 1.00 eq) in dichloromethane (10.0 mL) was added thionyl chloride (3.25 g, 27.3 mmol, 1.98 mL, 5.00 eq) at 0° C. The reaction was stirred at 25° C. for 2 h. The mixture was concentrated under reduced pressure to give a residue. The residue was diluted with water (10.0 mL) and extracted with ethyl acetate (3×35.0 mL). The combined organic layers were washed with brine (2×10.0 mL), dried over sodium sulfate, filtered, and concentrated under reduced pressure to afford 1-chloro-3-(chloromethyl)-2-methyl-5-nitrobenzene.

Step 4: To a solution of sodium hydride 60% purity (473 mg, 11.8 mmol, 2.00 eq) in tetrahydrofuran (15.0 mL) was added diethyl malonate (1.79 mL, 11.8 mmol, 2.00 eq) slowly at 0° C. After 1 h, 1-chloro-3-(chloromethyl)-2-methyl-5-nitrobenzene (1.30 g, 5.91 mmol, 1.00 eq) was added, and the reaction was stirred at 25° C. for 10 h. Water (10.0 mL) was added, and the mixture was extracted with ethyl acetate (3×35.0 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1) to afford diethyl 2-(3-chloro-2-methyl-5-nitrobenzyl)malonate.

Step 5: To a solution of diethyl 2-(3-chloro-2-methyl-5-nitrobenzyl)malonate (2.00 g, 5.82 mmol, 1.00 eq) in tetrahydrofuran (16.0 mL) was added sodium borohydride (1.10 g, 29.1 mmol, 5.00 eq) in portions at 0° C. Methanol (4.00 mL) was added, and the reaction was stirred at 25° C. for 10 h. Water (10.0 mL) was added, and the organic solvents were removed under reduced pressure. The aqueous layer was adjusted to pH=2.00 by addition of 1 N hydrochloric acid, and it was extracted with ethyl acetate (3×50.0 mL). The combined organic layers were washed with brine (15.0 mL), dried over magnesium sulfate, filtered, and concentrated under reduced pressure to afford 2-(3-chloro-2-methyl-5-nitrobenzyl)propane-1,3-diol.

Step 6: To a solution of 2-(3-chloro-2-methyl-5-nitrobenzyl)propane-1,3-diol (1.40 g, 5.39 mmol, 1.00 eq) in dichloromethane (10.0 mL) were added dimethoxymethane (715 μL, 8.09 mmol, 1.50 eq) and boron trifluoride diethyl etherate (1 mL, 8.09 mmol, 1.50 eq). The reaction was stirred at 25° C. for 1.5 h. The mixture was diluted with water (10.0 mL) and extracted with dichloromethane (3×35.0 mL). The combined organic layers were washed with brine (10.0 mL), dried over sodium sulfate, filtered, and concentrated under reduced pressure to give a residue. The residue was purified by silica gel column chromatography (petroleum ether/ethyl acetate=3/1) to afford 5-(3-chloro-2-methyl-5-nitrobenzyl)-1,3-dioxane.

Step 7: A mixture of 5-(3-chloro-2-methyl-5-nitrobenzyl)-1,3-dioxane (1.00 g, 3.68 mmol, 1.00 eq), ammonium chloride (197 mg, 3.68 mmol, 1.00 eq) and ferrous powder (1.03 g, 18.4 mmol, 5.00 eq) in ethanol (15.0 mL) and water (7.00 mL) was stirred at 90° C. for 10 h. The mixture was filtered and concentrated under reduced pressure to give a residue. Water (10.0 mL) was added, and the mixture was extracted with ethyl acetate (3×35.0 mL). The combined organic layers were washed with brine (10.0 mL), dried over sodium sulfate, filtered, and concentrated under reduced pressure to afford 3-((1,3-dioxan-5-yl)methyl)-5-chloro-4-methylaniline.

Step 8: To a solution of 3-((1,3-dioxan-5-yl)methyl)-5-chloro-4-methylaniline (0.20 g, 827 μmol, 1.00 eq) and potassium carbonate (229 mg, 1.65 mmol, 2.00 eq) in acetone (5.00 mL) was added phenyl chloroformate (124 μL, 993 μmol, 1.20 eq). The reaction was stirred at 25° C. for 10 h. The mixture was diluted with water (10.0 mL) and extracted with ethyl acetate (3×30.0 mL). The combined organic layers were washed with brine (10.0 mL), dried over sodium sulfate, filtered, and concentrated under reduced pressure to afford phenyl (3-((1,3-dioxan-5-yl)methyl)-5-chloro-4-methylphenyl)carbamate.

Compound 133: General procedure A with variant iii) was used for the preparation from compound VI-A employing phenyl (3-chloro-5-((4-(dimethylamino)piperidin-1-yl)methyl)-4-methylphenyl) carbamate. ¹H NMR (400 MHz, DMSO-d₆) δ=10.98 (s, 1H), 8.98 (s, 1H), 8.24 (s, 2H), 7.69-7.61 (m, 2H), 7.57 (d, J=0.8 Hz, 2H), 7.14 (d, J=2.0 Hz, 1H), 7.08-6.96 (m, 1H), 5.12 (dd, J=5.2, 13.2 Hz, 1H), 4.47-4.28 (m, 4H), 3.37 (s, 2H), 2.99-2.57 (m, 3H), 2.47-2.39 (m, 2H), 2.37 (s, 6H), 2.34-2.26 (m, 1H), 2.25 (s, 3H), 2.05-1.92 (m, 3H), 1.80 (br d, J=11.4 Hz, 2H), 1.44 (br dd, J=3.2, 11.8 Hz, 2H). MS (ESI) m/z 581.4 [M+H]⁺

### Preparation of phenyl (3-chloro-5-((4-(dimethylamino)piperidin-1-yl)methyl)-4-methylphenyl) carbamate

Step 1: To a solution of (3-chloro-2-methyl-5-nitrophenyl)methanol (0.80 g, 3.97 mmol, 1.00 eq) in dichloromethane (10.0 mL) was added thionyl chloride (1.44 mL, 19.8 mmol, 5.00 eq) at 0° C. The reaction was stirred at 25° C. for 2 h. The mixture was concentrated under reduced pressure to afford 1-chloro-3-(chloromethyl)-2-methyl-5-nitrobenzene.

Step 2: To a solution of 1-chloro-3-(chloromethyl)-2-methyl-5-nitrobenzene (0.90 g, 4.09 mmol, 1.00 eq) and triethylamine (1.42 mL, 10.2 mmol, 2.50 eq) in acetonitrile (10.0 mL) was added N,N-dimethylpiperidin-4-amine (655 mg, 5.11 mmol, 1.25 eq). The reaction was stirred at 25° C. for 10 h. The mixture was filtered and concentrated under reduced pressure to afford 1-(3-chloro-2-methyl-5-nitrobenzyl)-N,N-dimethylpiperidin-4-amine.

Step 3: A mixture of 1-(3-chloro-2-methyl-5-nitrobenzyl)-N,N-dimethylpiperidin-4-amine (1.30 g, 4.17 mmol, 1.00 eq), ammonium chloride (223 mg, 4.17 mmol, 1.00 eq) and ferrous powder (1.16 g, 20.9 mmol, 5.00 eq) in ethanol (20.0 mL) and water (10.0 mL) was stirred at 90° C. for 10 h. The mixture was filtered and concentrated under reduced pressure to give a residue. The residue was purified was by reversed phase column chromatography and lyophilized to afford 1-(5-amino-3-chloro-2-methylbenzyl)-N,N-dimethylpiperidin-4-amine.

Step 4: To a solution of 1-(5-amino-3-chloro-2-methylbenzyl)-N,N-dimethylpiperidin-4-amine (0.60 g, 2.13 mmol, 1.00 eq) and potassium carbonate (588 mg, 4.26 mmol, 2.00 eq) in acetone (10.0 mL) was added phenyl chloroformate (400 mg, 2.55 mmol, 320 μL, 1.20 eq). The reaction was stirred at 25° C. for 1 h. The mixture was filtered and concentrated and the obtained residue was purified by standard methods to afford phenyl (3-chloro-5-((4-(dimethylamino)piperidin-1-yl)methyl)-4-methylphenyl)carbamate.

Compound 134: General procedure A with variant iii) was used for the preparation from compound VI-A employing phenyl (3-chloro-5-(2-(dimethylamino)ethoxy)-4-methylphenyl)carbamate. ¹H NMR (400 MHz, DMSO-d₆) δ=11.0 (s, 1H), 10.6 (br s, 1H), 9.28 (s, 1H), 7.65 (s, 1H), 7.57 (s, 2H), 7.25-7.07 (m, 3H), 5.12 (dd, J=5.1, 13.2 Hz, 1H), 4.49-4.37 (m, 3H), 4.35-4.26 (m, 3H), 3.57-3.49 (m, 2H), 2.97-2.88 (m, 1H), 2.86 (s, 3H), 2.85 (s, 3H), 2.60 (br d, J=16.7 Hz, 1H), 2.46-2.33 (m, 1H), 2.16 (s, 3H), 2.05-1.95 (m, 1H). MS (ESI) m/z 528.4 [M+H]⁺

### Preparation of phenyl (3-chloro-5-(2-(dimethylamino)ethoxy)-4-methylphenyl)carbamate

Step 1: To a solution of methyl 3-chloro-5-hydroxy-4-methylbenzoate (600 mg, 2.99 mmol, 1.00 eq) in acetonitrile (6.00 mL) was added potassium iodide (49.7 mg, 299 μmol, 0.10 eq), potassium carbonate (1.65 g, 12.0 mmol, 4.00 eq) and 2-chloro-N,N-dimethylethanamine (862 mg, 5.98 mmol, 2.00 eq, hydrochloric acid). The reaction was stirred at 80° C. for 12 h. The mixture was filtered and concentrated under reduced pressure to give a residue. The residue was purified by silica gel column chromatography (dichloromethane/methanol=1/0 to 10/1) to afford methyl 3-chloro-5-(2-(dimethylamino)ethoxy)-4-methylbenzoate.

Step 2: To a solution of methyl 3-chloro-5-(2-(dimethylamino)ethoxy)-4-methylbenzoate (500 mg, 1.84 mmol, 1.00 eq) in tetrahydrofuran (1.50 mL) and water (0.50 mL) was added lithium hydroxide monohydrate (232 mg, 5.52 mmol, 3.00 eq). The reaction was stirred at 20° C. for 12 h. The mixture was concentrated under reduced pressure to give a residue. The residue was purified by reversed phase column chromatography and lyophilized to give solid. Water (20.0 mL) was added followed by hydrochloric acid (0.20 mL), and the mixture was lyophilized to afford 3-chloro-5-(2-(dimethylamino)ethoxy)-4-methylbenzoic acid.

Step 3: To a solution of 3-chloro-5-(2-(dimethylamino)ethoxy)-4-methylbenzoic acid (300 mg, 1.16 mmol, 1.00 eq) in toluene (1.00 mL) was added triethylamine (259 mg, 2.56 mmol, 2.20 eq) and diphenylphosphoryl azide (352 mg, 1.28 mmol, 1.10 eq). The reaction was stirred at 20° C. for 10 min, then phenol (548 mg, 5.82 mmol, 5.00 eq) was added. The reaction was stirred at 100° C. for 30 min. The mixture was concentrated and the obtained residue was purified by standard methods to afford phenyl (3-chloro-5-(2-(dimethylamino)ethoxy)-4-methylphenyl)carbamate.

Compound 135: General procedure A with variant iii) was used for the preparation from compound VI-A employing phenyl (3-(2-hydroxypropan-2-yl)phenyl)carbamate. ¹H NMR (400 MHz, DMSO-d₆) δ=11.00 (s, 1H), 8.65 (s, 1H), 7.67 (s, 1H), 7.57 (s, 2H), 7.46 (s, 1H), 7.33 (br d, J=7.8 Hz, 1H), 7.14 (t, J=7.8 Hz, 1H), 6.99 (d, J=7.8 Hz, 1H), 6.73 (t, J=5.8 Hz, 1H), 5.12 (dd, J=5.0, 13.2 Hz, 1H), 4.94 (s, 1H), 4.52-4.24 (m, 4H), 2.99-2.85 (m, 1H), 2.62 (br d, J=2.4 Hz, 1H), 2.43-2.31 (m, 1H), 2.07-1.94 (m, 1H), 1.39 (s, 6H). MS (ESI) m/z 449.4 [M−H]⁻

### Preparation of phenyl (3-(2-hydroxypropan-2-yl)phenyl)carbamate

Step 1: A mixture of 1-(3-nitrophenyl)ethanone (2.00 g, 12.1 mmol, 1.00 eq), ammonium chloride (648 mg, 12.1 mmol, 1.00 eq) and ferrous powder (3.38 g, 60.6 mmol, 5.00 eq) in ethanol (20.0 mL) and water (10.0 mL) was stirred at 90° C. for 10 h. The mixture was filtered, and the filtrate was extracted with ethyl acetate (3×50.0 mL). The combined organic layers were washed with brine (20.0 mL), dried over sodium sulfate, filtered, and concentrated under reduced pressure to afford 1-(3-aminophenyl)ethanone.

Step 2: To a solution of 1-(3-aminophenyl)ethanone (1.50 g, 11.1 mmol, 1.00 eq) in tetrahydrofuran (15.0 mL) was added methylmagnesium bromide (3.00 M, 11.1 mL, 3.00 eq) dropwise at 0° C. The reaction was stirred at 25° C. for 2 h. The mixture was quenched by addition saturated ammonium chloride (8.00 mL), and then diluted with water (5.00 mL) and extracted with ethyl acetate (3×30.0 mL). The combined organic layers were washed with brine (10.0 mL), dried over sodium sulfate, filtered, and concentrated under reduced pressure to give a residue. The residue was purified by reversed phase preparative HPLC to afford 2-(3-aminophenyl)propan-2-ol.

Step 3: To a solution of 2-(3-aminophenyl)propan-2-ol (0.10 g, 661 μmol, 1.00 eq) and 2,6-dimethylpyridine (84.7 μL, 727 μmol, 1.10 eq) in tetrahydrofuran (0.50 mL) and trichloromethane (0.50 mL) was added phenyl chloroformate (82.8 μL, 661 μmol, 1.00 eq) slowly at 0° C. The reaction was stirred at 25° C. for 2 h. The mixture was diluted with water (5.00 mL) and extracted with dichloromethane (3×25.0 mL). The combined organic layers were washed with brine (8.00 mL), dried over sodium sulfate, filtered, and concentrated under reduced pressure to afford phenyl (3-(2-hydroxypropan-2-yl)phenyl)carbamate.

Compound 136: A mixture of 2-(dimethylamino)acetic acid (75.7 mg, 734 μmol, 1.50 eq), 4-dimethylaminopyridine (6.0 mg, 48.9 μmol, 0.10 eq) and N,N′-methanediylidenedicyclohexanamine (148 μL, 734 μmol, 1.50 eq) in dimethylformamide (5.00 mL) was stirred at 20° C. for 30 min, then 1-((2-(2,6-dioxopiperidin-3-yl)-3-oxoisoindolin-5-yl)methyl)-3-(3-hydroxyphenyl)urea (described for compound 110) (200 mg, 489 μmol, 1.00 eq) was added. The reaction was stirred at 20° C. for 12 h, then at 40° C. for 4 h. The mixture was filtered, and the filtrate was purified by standard methods to afford Compound 136. ¹H NMR (400 MHz, DMSO-d₆) δ=10.98 (s, 1H), 10.44 (br s, 1H), 9.38-9.32 (m, 1H), 7.66 (s, 1H), 7.58-7.57 (m, 3H), 7.32-7.28 (m, 1H), 7.17-7.12 (m, 2H), 6.75-6.73 (m, 1H), 5.13-5.09 (m, 1H), 4.49-4.33 (m, 6H), 2.95-2.87 (m, 7H), 2.62-2.58 (m, 1H), 2.42-2.38 (m, 1H), 2.02-2.00 (m, 1H). MS (ESI) m/z 494.2 [M+H]⁺

Compound 137: General procedure A with variant iii) was used for the preparation from compound VI-A employing phenyl (6-chloro-5-methylpyridin-2-yl)carbamate. ¹H NMR (400 MHz, DMSO-d₆) δ=10.98 (br s, 1H), 9.44 (s, 1H), 7.69-7.64 (m, 2H), 7.62-7.59 (m, 1H), 7.57 (s, 2H), 7.48 (br t, J=5.8 Hz, 1H), 5.11 (dd, J=5.1, 13.3 Hz, 1H), 4.48-4.45 (m, 2H), 4.41 (s, 1H), 4.35-4.27 (m, 1H), 2.96-2.86 (m, 1H), 2.63-2.56 (m, 1H), 2.45-2.36 (m, 1H), 2.22 (s, 3H), 2.04-1.96 (m, 1H). MS (ESI) m/z 442.1 [M+H]⁺

Preparation of phenyl (6-chloro-5-methylpyridin-2-yl)carbamate: To a solution of 6-chloro-5-methyl-pyridin-2-amine (300 mg, 2.10 mmol, 1.00 eq) in dichloromethane (15.0 mL) was added pyridine (0.25 mL, 3.16 mmol, 1.50 eq) and phenyl chloroformate (0.26 mL, 2.10 mmol, 1.00 eq) at 0° C. The reaction was stirred at 20° C. for 2 h. The mixture was diluted with water (20.0 mL) and extracted with ethyl acetate (3×30.0 mL). The combined organic layers were washed with brine (3×20.0 mL), dried over anhydrous sodium sulfate, filtered, and concentrated under reduced pressure to afford phenyl (6-chloro-5-methylpyridin-2-yl)carbamate d.

Compound 138: General procedure A with variant iii) was used for the preparation from compound VI-A employing phenyl (3-(3-oxa-8-azabicyclo[3.2.1]octan-8-ylmethyl)-5-chloro-4-methylphenyl)carbamate. ¹H NMR (400 MHz, DMSO-d₆) δ=10.98 (s, 1H), 8.74 (s, 1H), 7.66 (s, 1H), 7.64 (d, J=2.3 Hz, 1H), 7.56 (d, J=1.0 Hz, 2H), 7.20 (d, J=2.1 Hz, 1H), 6.76 (t, J=6.1 Hz, 1H), 5.11 (dd, J=5.1, 13.3 Hz, 1H), 4.48-4.25 (m, 4H), 3.55-3.49 (m, 2H), 3.43-3.39 (m, 2H), 3.35-3.35 (m, 2H), 2.97 (br s, 2H), 2.95-2.84 (m, 1H), 2.63-2.56 (m, 1H), 2.45-2.34 (m, 1H), 2.28 (s, 3H), 2.04-1.90 (m, 3H), 1.82-1.68 (m, 2H). MS (ESI) m/z 566.2 [M+H]⁺

### Preparation of phenyl (3-(3-oxa-8-azabicyclo[3.2.1]octan-8-ylmethyl)-5-chloro-4-methylphenyl)carbamate

Step 1: To a solution of 1-(bromomethyl)-3-chloro-2-methyl-5-nitrobenzene (500 mg, 1.89 mmol, 1.00 eq) in acetonitrile (10.0 mL) was added 3-oxa-8-azabicyclo[3.2.1]octane hydrochloride (283 mg, 1.89 mmol, 1.00 eq, hydrochloride), potassium carbonate (523 mg, 3.78 mmol, 2.00 eq) and potassium iodide (31.4 mg, 189 μmol, 0.10 eq). The reaction was stirred at 80° C. for 12 h. The mixture was concentrated under reduced pressure to afford a residue. The residue was diluted with water (10.0 mL) and extracted with ethyl acetate (20.0 mL). The organic layer was washed with water (10.0 mL), dried over sodium sulfate, filtered, and the filtrate was concentrated to afford a residue. The residue was purified by silica gel column chromatography (petroleum ether/ethyl acetate=1/0 to 0/1 then dichloromethane/methanol=10/1) to afford 8-(3-chloro-2-methyl-5-nitrobenzyl)-3-oxa-8-azabicyclo[3.2.1]octane.

Step 2: To a solution of 8-(3-chloro-2-methyl-5-nitrobenzyl)-3-oxa-8-azabicyclo[3.2.1]octane (390 mg, 1.31 mmol, 100 eq) and ammonium chloride (492 mg, 9.20 mmol, 7.00 eq) in methanol (5.00 mL) and water (5.00 mL) was added iron powder (514 mg, 9.20 mmol, 7.00 eq). The reaction was stirred at 80° C. for 1 h. The mixture was concentrated under reduced pressure to give a residue. The residue was diluted with water (20 mL) and basified to pH=10 with sodium hydroxide. The mixture was extracted with ethyl acetate (50.0 mL). The organic layer was washed with water (20.0 mL), dried over sodium sulfate, filtered, and the filtrate was concentrated under reduced pressure to afford 3-(3-oxa-8-azabicyclo[3.2.1]octan-8-ylmethyl)-5-chloro-4-methylaniline.

Step 3: To a solution of 3-(3-oxa-8-azabicyclo[3.2.1]octan-8-ylmethyl)-5-chloro-4-methylaniline (230 mg, 862 μmol, 1.00 eq) and potassium carbonate (119 mg, 862 μmol, 1.00 eq) in acetone (3.00 mL) was added phenyl chloroformate (0.11 mL, 862 μmol, 1.00 eq) dropwise. The reaction was stirred at 20° C. for 1 h. The mixture was filtered, and the filtrate was concentrated and the obtained residue was purified by standard methods to afford phenyl (3-(3-oxa-8-azabicyclo[3.2.1]octan-8-ylmethyl)-5-chloro-4-methylphenyl)carbamate.

Compound 139: General procedure A with variant iii) was used for the preparation from compound VI-A employing phenyl (3-(2-oxa-5-azabicyclo[2.2.1]heptan-5-ylmethyl)-5-chloro-4-methylphenyl) carbamate. ¹H NMR (400 MHz, DMSO-d₆+D₂O, T=80° C.) δ=7.68-7.62 (m, 2H), 7.58-7.51 (m, 3H), 5.00 (dd, J=5.3, 13.1 Hz, 1H), 4.67 (s, 1H), 4.50-4.32 (m, 7H), 4.26-4.12 (m, 1H), 3.73 (br d, J=9.8 Hz, 1H), 3.37-3.28 (m, 2H), 2.94-2.77 (m, 1H), 2.71-2.59 (m, 1H), 2.46-2.34 (m, 2H), 2.34 (s, 3H), 2.12-2.01 (m, 2H). MS (ESI) m/z 552.3 [M+H]⁺

### Preparation of phenyl (3-(2-oxa-5-azabicyclo[2.2.1]heptan-5-ylmethyl)-5-chloro-4-methylphenyl) carbamate

Step 1: To a solution of 2-oxa-5-azabicyclo[2.2.1]heptane hydrochloride (308 mg, 2.27 mmol, 1.00 eq, hydrochloride) in acetonitrile (10.0 mL) was added 1-(bromomethyl)-3-chloro-2-methyl-5-nitrobenzene (600 mg, 2.27 mmol, 1.00 eq), potassium carbonate (627 mg, 4.54 mmol, 2.00 eq) and potassium iodide (37.6 mg, 227 μmol, 0.10 eq). The reaction was stirred at 80° C. for 12 h. The mixture was diluted with water (50.0 mL) and extracted with ethyl acetate (3×40.0 mL). The combined organic layers were washed with brine (20.0 mL), dried over sodium sulfate, filtered, and concentrated under reduced pressure to give a residue. The residue was triturated with petroleum ether (2.00 mL) and filtered. The filter cake was dissolved in ethyl acetate (50.0 mL) and silica gel was added. The mixture was filtered, and the filtrate was concentrated under reduced pressure to afford 5-(3-chloro-2-methyl-5-nitrobenzyl)-2-oxa-5-azabicyclo[2.2.1]heptane.

Step 2: To a solution of 5-(3-chloro-2-methyl-5-nitrobenzyl)-2-oxa-5-azabicyclo[2.2.1]heptane (500 mg, 1.77 mmol, 1.00 eq), ammonium chloride (662 mg, 12.4 mmol, 7.00 eq) in methanol (8.00 mL) and water (2.00 mL) was added iron powder (691 mg, 12.4 mmol, 7.00 eq) in portions. The reaction was stirred at 70° C. for 2 h. The mixture was filtered, and the filtrate was concentrated under reduced pressure to give a residue. The residue was diluted with water (60.0 mL) and extracted with ethyl acetate (3×40.0 mL). The combined organic layers were washed with brine (30.0 mL), dried over sodium sulfate, filtered, and concentrated under reduced pressure to give a residue. The residue was purified by silica gel column chromatography (petroleum ether/ethyl acetate=2/1 to 0/1) to give 3-(2-oxa-5-azabicyclo[2.2.1]heptan-5-ylmethyl)-5-chloro-4-methylaniline.

Step 3: To a solution of 3-(2-oxa-5-azabicyclo[2.2.1]heptan-5-ylmethyl)-5-chloro-4-methylaniline (300 mg, 1.19 mmol, 1.00 eq) and phenyl chloroformate (0.15 mL, 1.19 mmol, 1.00 eq) in acetone (3.00 mL) was added potassium carbonate (492 mg, 3.56 mmol, 3.00 eq) in portions. The reaction was stirred at 15° C. for 2 h. Water (30.0 mL) was added, and the mixture was extracted with ethyl acetate (3×30.0 mL). The combined organic layers were washed with brine (20.0 mL), dried over anhydrous sodium sulfate, filtered, and concentrated and the obtained residue was purified by standard methods to afford phenyl (3-(2-oxa-5-azabicyclo[2.2.1]heptan-5-ylmethyl)-5-chloro-4-methylphenyl)carbamate.

Compound 140: General procedure A with variant iii) was used for the preparation from compound VI-A employing phenyl (3-(8-oxa-3-azabicyclo[3.2.1]octan-3-ylmethyl)-5-chloro-4-methylphenyl)carbamate. ¹H NMR (400 MHz, DMSO-d₆) δ=10.98 (br s, 1H), 8.77 (s, 1H), 8.30 (s, 1H), 7.69-7.60 (m, 2H), 7.56 (s, 2H), 7.11 (d, J=1.9 Hz, 1H), 6.85 (br t, J=5.9 Hz, 1H), 5.11 (dd, J=5.0, 13.3 Hz, 1H), 4.48-4.36 (m, 3H), 4.35-4.26 (m, 1H), 4.19 (br s, 2H), 2.98-2.85 (m, 1H), 2.68-2.52 (m, 3H), 2.47-2.33 (m, 3H), 2.26 (s, 3H), 2.19 (br d, J=10.6 Hz, 2H), 2.06-1.92 (m, 1H), 1.84-1.75 (m, 2H), 1.75-1.63 (m, 2H). MS (ESI) m/z 566.1 [M+H]⁺

### Preparation of phenyl (3-(8-oxa-3-azabicyclo[3.2.1]octan-3-ylmethyl)-5-chloro-4-methylphenyl)carbamate

Step 1: To a solution of 8-oxa-3-azabicyclo[3.2.1]octane (400 mg, 3.53 mmol, 1.00 eq) and triethylamine (0.74 mL, 5.30 mmol, 1.50 eq) in tetrahydrofuran (15.0 mL) was added 1-(bromomethyl)-3-chloro-2-methyl-5-nitrobenzene (842 mg, 3.18 mmol, 0.90 eq). The reaction was stirred at 20° C. for 6 h. The mixture was concentrated under reduced pressure to afford a residue. The residue was purified by preparative TLC (petroleum ether/ethyl acetate=5/1) to afford 3-(3-chloro-2-methyl-5-nitrobenzyl)-8-oxa-3-azabicyclo[3.2.1]octane.

Step 2: A mixture of 3-(3-chloro-2-methyl-5-nitrobenzyl)-8-oxa-3-azabicyclo[3.2.1]octane (750 mg, 2.53 mmol, 1.00 eq), ammonium chloride (135 mg, 2.53 mmol, 1.00 eq) and iron powder (706 mg, 12.6 mmol, 5.00 eq) in ethanol (20.0 mL) and water (10.0 mL) was stirred at 90° C. for 3 h. The mixture was filtered and concentrated under reduced pressure to give a residue. The residue was purified by preparative TLC (petroleum ether/ethyl acetate=5/1) to afford 3-(8-oxa-3-azabicyclo[3.2.1]octan-3-ylmethyl)-5-chloro-4-methylaniline.

Step 3: To a solution of 3-(8-oxa-3-azabicyclo[3.2.1]octan-3-ylmethyl)-5-chloro-4-methylaniline (300 mg, 1.12 mmol, 1.00 eq) and potassium carbonate (466 mg, 3.37 mmol, 3.00 eq) in acetone (3.00 mL) was added phenyl chloroformate (0.14 mL, 1.12 mmol, 1.00 eq) dropwise. The reaction was stirred at 15° C. for 2 h. Water (50.0 mL) was added, and the mixture was extracted with ethyl acetate (3×40.0 mL). The combined organic layers were washed with brine (30.0 mL), dried over anhydrous sodium sulfate, filtered, and concentrated under reduced pressure to afford phenyl (3-(8-oxa-3-azabicyclo[3.2.1]octan-3-ylmethyl)-5-chloro-4-methylphenyl)carbamate.

Compound 141: General procedure A with variant iii) was used for the preparation from compound VI-A employing phenyl (3-chloro-4-methyl-5-((2-methyl-1,3-dioxan-5-yl)methyl)phenyl)carbamate. ¹H NMR (400 MHz, DMSO-d₆) δ=10.98 (s, 1H), 8.73 (s, 1H), 7.66 (s, 1H), 7.60-7.49 (m, 3H), 7.01 (d, J=2.0 Hz, 1H), 6.85 (br s, 1H), 5.12 (dd, J=5.2, 13.3 Hz, 1H), 4.61 (q, J=5.0 Hz, 1H), 4.49-4.27 (m, 4H), 3.86 (dd, J=4.4, 11.6 Hz, 2H), 3.42 (t, J=11.2 Hz, 2H), 2.99-2.84 (m, 1H), 2.60 (td, J=2.0, 15.2 Hz, 1H), 2.40 (dd, J=4.4, 13.0 Hz, 1H), 2.34 (d, J=7.2 Hz, 2H), 2.20 (s, 3H), 2.06-1.95 (m, 2H), 1.16 (d, J=5.0 Hz, 3H). MS (ESI) m/z 555.3 [M+H]⁺

### Preparation of phenyl (3-chloro-4-methyl-5-((2-methyl-1,3-dioxan-5-yl)methyl)phenyl)carbamate

Step 1: To a solution of 2-(3-chloro-2-methyl-5-nitrobenzyl)propane-1,3-diol (1.00 g, 3.85 mmol, 1.00 eq) (described for compound 132) and 1,1-dimethoxyethane (611 μL, 5.78 mmol, 1.50 eq) in dichloromethane (10.0 mL) was added boron trifluoride diethyl etherate (713 μL, 5.78 mmol, 1.50 eq). The reaction was stirred at 25° C. for 1.5 h. The mixture was diluted with water (10.0 mL) and extracted with ethyl acetate (3×35.0 mL). The combined organic layers were washed with brine (10.0 mL), dried over magnesium sulfate, filtered, and concentrated under reduced pressure to afford 5-(3-chloro-2-methyl-5-nitrobenzyl)-2-methyl-1,3-dioxane.

Step 2: A mixture of 5-(3-chloro-2-methyl-5-nitrobenzyl)-2-methyl-1,3-dioxane (1.10 g, 3.85 mmol, 1.00 eq), ferrous powder (1.07 g, 19.3 mmol, 5.00 eq) and ammonium chloride (206 mg, 3.85 mmol, 1.00 eq) in ethanol (10.0 mL) and water (5.00 mL) was stirred at 90° C. for 10 h. The mixture was filtered and concentrated under reduced pressure to give a residue. The residue was purified by reversed phase column chromatography and lyophilized to afford 3-chloro-4-methyl-5-((2-methyl-1,3-dioxan-5-yl)methyl)aniline.

Step 3: To a solution of 3-chloro-4-methyl-5-((2-methyl-1,3-dioxan-5-yl)methyl)aniline (0.60 g, 2.35 mmol, 1.00 eq) and potassium carbonate (649 mg, 4.69 mmol, 2.00 eq) in acetone (10.0 mL) was added phenyl chloroformate (0.35 mL, 2.82 mmol, 1.20 eq). The reaction was stirred at 25° C. for 2 h. The mixture was diluted with water (10.0 mL) and extracted with ethyl acetate (3×35.0 mL). The combined organic layers were washed with brine (10.0 mL), dried over sodium sulfate, filtered, and concentrated under reduced pressure to afford phenyl (3-chloro-4-methyl-5-((2-methyl-1,3-dioxan-5-yl)methyl) phenyl)carbamate.

Compound 142: General procedure A with variant iii) was used for the preparation from compound VI-A employing phenyl (3-chloro-5-((2,2-dimethyl-1,3-dioxan-5-yl)methyl)-4-methylphenyl)carbamate. ¹H NMR (400 MHz, DMSO-d₆) δ=11.24-10.65 (m, 1H), 8.73 (s, 1H), 7.66 (s, 1H), 7.60-7.53 (m, 3H), 7.01 (d, J=2.0 Hz, 1H), 6.80 (t, J=6.2 Hz, 1H), 5.12 (dd, J=5.2, 13.2 Hz, 1H), 4.50-4.26 (m, 4H), 3.75 (dd, J=4.2, 11.6 Hz, 2H), 3.57 (dd, J=7.6, 11.6 Hz, 2H), 2.97-2.85 (m, 1H), 2.65-2.56 (m, 3H), 2.40 (dd, J=4.4, 12.8 Hz, 1H), 2.22 (s, 3H), 2.06-1.95 (m, 1H), 1.80 (td, J=3.8, 7.4 Hz, 1H), 1.37 (s, 3H), 1.30 (s, 3H). MS (ESI) m/z 569.2 [M+H]⁺

### Preparation of phenyl (3-chloro-5-((2,2-dimethyl-1,3-dioxan-5-yl)methyl)-4-methylphenyl)carbamate

Step 1: A mixture of 2-(3-chloro-2-methyl-5-nitrobenzyl)propane-1,3-diol (0.82 g, 3.16 mmol, 1.00 eq) (described for compound 132), 2,2-dimethoxypropane (0.43 mL, 3.47 mmol, 1.10 eq) and indium (III) trifluoromethanesulfonate (177 mg, 316 μmol, 0.10 eq) was stirred at 25° C. for 0.5 h. The mixture was diluted with water (20.0 mL) and extracted with ethyl acetate (3×55.0 mL). The combined organic layers were washed with brine (15.0 mL), dried over magnesium sulfate, filtered, and concentrated under reduced pressure to afford 5-(3-chloro-2-methyl-5-nitrobenzyl)-2,2-dimethyl-1,3-dioxane.

Step 2: A mixture of 5-(3-chloro-2-methyl-5-nitrobenzyl)-2,2-dimethyl-1,3-dioxane (1.00 g, 3.34 mmol, 1.00 eq), ferrous powder (932 mg, 16.7 mmol, 5.00 eq) and ammonium chloride (178 mg, 3.34 mmol, 1.00 eq) in ethanol (10.0 mL) and water (5.00 mL) was stirred at 50° C. for 10 h. The mixture was concentrated under reduced pressure to give a residue. The residue was diluted with water (15.0 mL) and extracted with ethyl acetate (3×35.0 mL). The combined organic layers were washed with brine (10.0 mL), dried over sodium sulfate, filtered, and concentrated under reduced pressure to give a residue. The residue was purified by silica gel column chromatography (petroleum ether/ethyl acetate=3/1) to afford 3-chloro-5-((2,2-dimethyl-1,3-dioxan-5-yl)methyl)-4-methylaniline.

Step 3: To a solution of 3-chloro-5-((2,2-dimethyl-1,3-dioxan-5-yl)methyl)-4-methylaniline (0.40 g, 1.48 mmol, 1.00 eq) and potassium carbonate (410 mg, 2.97 mmol, 2.00 eq) in acetone (5.00 mL) was added phenyl chloroformate (0.22 mL, 1.78 mmol, 1.20 eq). The reaction was stirred at 25° C. for 2 h. The mixture was diluted with water (15.0 mL) and extracted with ethyl acetate (3×35.0 mL). The combined organic layers were washed with brine (10.0 mL), dried over sodium sulfate, filtered, and concentrated under reduced pressure to afford phenyl (3-chloro-5-((2,2-dimethyl-1,3-dioxan-5-yl)methyl)-4-methylphenyl)carbamate.

Compound 143: General procedure A with variant iii) was used for the preparation from compound VI-A employing phenyl(3-chloro-4-methyl-5-(trifluoromethoxy)phenyl)carbamate. ¹H NMR (400 MHz, DMSO-d₆) δ=10.98 (s, 1H), 9.10 (s, 1H), 7.67 (s, 1H), 7.58-7.55 (m, 3H), 7.54 (d, J=1.2 Hz, 1H), 7.00 (t, J=6.0 Hz, 1H), 5.12 (dd, J=5.2, 13.6 Hz, 1H), 4.47-4.39 (m, 3H), 4.34-4.28 (m, 1H), 2.96-2.87 (m, 1H), 2.64-2.57 (m, 1H), 2.47-2.36 (m, 1H), 2.20 (s, 3H), 2.00 (dtd, J=2.0, 5.2, 12.4 Hz, 1H). MS (ESI) m/z 525.0 [M+H]⁺

### Preparation of phenyl(3-chloro-4-methyl-5-(trifluoromethoxy)phenyl)carbamate

Step 1: To a solution of 3-chloro-5-(trifluoromethoxy)aniline (1.60 g, 7.56 mmol, 1.00 eq) in dimethyl formamide (20.0 mL) was added N-bromosuccinimide (1.35 g, 7.56 mmol, 1.00 eq). The reaction was stirred at 25° C. for 12 h. The mixture was poured into water (120 mL) and stirred for 10 min. The aqueous phase was extracted with ethyl acetate (3×60.0 mL). The combined organic layers were washed with brine (100 mL), dried over anhydrous sodium sulfate, filtered, and concentrated under reduced pressure to afford 4-bromo-3-chloro-5-(trifluoromethoxy) aniline.

Step 2: To a mixture of 4-bromo-3-chloro-5-(trifluoromethoxy)aniline (1.00 g, 3.44 mmol, 1.00 eq), methylboronic acid (824 mg, 13.8 mmol, 4.00 eq) and caesium carbonate (3.37 g, 10.3 mmol, 3.00 eq) in dioxane (10.0 mL) was added [1,1′-bis(diphenylphosphino)ferrocene]dichloropalladium(II) (252 mg, 344 μmol, 0.100 eq) under nitrogen. The reaction was stirred at 100° C. for 3 h. The mixture was concentrated under reduced pressure to give a residue. The residue was purified by silica gel column chromatography (petroleum ether/ethyl acetate=20/1 to 2/1) to afford 3-chloro-4-methyl-5-(trifluoromethoxy)aniline.

Step 3: To a solution of 3-chloro-4-methyl-5-(trifluoromethoxy)aniline (600 mg, 2.66 mmol, 1.00 eq) and pyridine (0.64 mL, 7.98 mmol, 3.00 eq) in acetonitrile (10.0 mL) was added phenyl chloroformate (0.50 mL, 3.99 mmol, 1.50 eq). The reaction was stirred at 20° C. for 3 h. The mixture was concentrated under reduced pressure to give a residue. The residue was purified by silica gel column chromatography (petroleum ether/ethyl acetate=1/0 to 40/1) to afford phenyl (3-chloro-4-methyl-5-(trifluoromethoxy) phenyl)carbamate

Compound 144: General procedure A with variant iii) was used for the preparation from compound VI-A employing phenyl(4-methyl-3-(trifluoromethoxy)phenyl)carbamate. ¹H NMR (400 MHz, DMSO-d₆) δ=10.98 (s, 1H), 8.88 (s, 1H), 7.69-7.63 (m, 2H), 7.60-7.53 (m, 2H), 7.25-7.20 (m, 1H), 7.20-7.14 (m, 1H), 6.83 (t, J=6.0 Hz, 1H), 5.12 (dd, J=5.1, 13.3 Hz, 1H), 4.46-4.28 (m, 4H), 2.97-2.86 (m, 1H), 2.63-2.57 (m, 1H), 2.40 (dq, J=4.5, 13.2 Hz, 1H), 2.18 (s, 3H), 2.00 (dtd, J=2.1, 5.2, 12.6 Hz, 1H). MS (ESI) m/z 491.2 [M+H]⁺

### Preparation of phenyl(4-methyl-3-(trifluoromethoxy)phenyl)carbamate

Step 1: To a solution of 2-methyl-5-nitro-phenol (2.00 g, 13.1 mmol, 1.00 eq), silver trifluoromethanesulfonate (16.8 g, 65.3 mmol, 5.00 eq), 1-(chloromethyl)-4-fluoro-1,4-diazoniabicyclo[2.2.2]octane ditetrafluoroborate (9.25 g, 26.1 mmol, 2.00 eq), N-fluorobenzenesulfonimide (8.24 g, 26.1 mmol, 2.00 eq), and caesium fluoride (11.9 g, 78.4 mmol, 6.00 eq) in toluene (100 mL) was added trimethyl(trifluoromethyl)silane (9.29 g, 65.3 mmol, 5.00 eq) and 2-fluoropyridine (5.61 mL, 65.3 mmol, 5.00 eq) under nitrogen atmosphere. The reaction was stirred at 20° C. for 12 h under nitrogen. The mixture was filtered, and the filtrate was concentrated under reduced pressure to afford a residue. The residue was diluted with water (100 mL) and extracted with petroleum ether/ethyl acetate (10/1, 100 mL). The organic layer was washed with water (20.0 mL), dried over anhydrous sodium sulfate, filtered, and the filtrate was concentrated under reduced pressure to give a residue. The residue was purified by silica gel column chromatography (petroleum ether/ethyl acetate=1/0 to 20/1) to afford 1-methyl-4-nitro-2-(trifluoromethoxy)benzene.

Step 2: To a solution of 1-methyl-4-nitro-2-(trifluoromethoxy)benzene (1.60 g, 7.24 mmol, 1.00 eq) in methanol (20.0 mL) and water (20.0 mL) was added iron powder (2.83 g, 50.7 mmol, 7.00 eq) and ammonium chloride (2.71 g, 50.7 mmol, 7.00 eq). The reaction was stirred at 80° C. for 2 h. The mixture was concentrated under reduced pressure to give a residue. Ethyl acetate (50.0 mL) was added to the residue, and the mixture was washed with water (20.0 mL). The organic layer was dried over anhydrous sodium sulfate, filtered, and the filtrate was concentrated under reduced pressure to give a residue. The residue was purified by silica gel column chromatography (petroleum ether/ethyl acetate=1/0 to 5/1) to afford 4-methyl-3-(trifluoromethoxy)aniline.

Step 3: To a mixture of 4-methyl-3-(trifluoromethoxy)aniline (200 mg, 1.05 mmol, 1.00 eq) and potassium carbonate (174 mg, 1.26 mmol, 1.20 eq) in acetone (4.00 mL) was added phenyl chloroformate (180 mg, 1.15 mmol, 1.10 eq). The reaction was stirred at 20° C. for 1 h. The mixture was diluted with water (3.00 mL), extracted with ethyl acetate (5.00 mL), dried over anhydrous sodium sulfate, filtered, and the filtrate was concentrated under reduced pressure to afford phenyl(4-methyl-3-(trifluoromethoxy)phenyl)carbamate.

Compound 145: General procedure A with variant iii) was used for the preparation from compound VI-A employing phenyl (3-chloro-4-methyl-5-((2-methyl-1,3-dioxan-5-yl)methyl)phenyl)carbamate. ¹H NMR (400 MHz, DMSO-d₆) δ=10.99 (br s, 1H), 8.86 (s, 1H), 8.44 (s, 1H), 7.69-7.59 (m, 2H), 7.56 (d, J=0.8 Hz, 2H), 6.98 (d, J=2.0 Hz, 1H), 6.85 (t, J=6.0 Hz, 1H), 5.12 (dd, J=5.2, 13.4 Hz, 1H), 4.69 (q, J=5.0 Hz, 1H), 4.50-4.26 (m, 4H), 3.87-3.77 (m, 2H), 3.70 (d, J=11.0 Hz, 2H), 2.99-2.82 (m, 3H), 2.66-2.56 (m, 1H), 2.46-2.32 (m, 1H), 2.23 (s, 3H), 2.06-1.96 (m, 1H), 1.54 (br t, J=7.2 Hz, 1H), 1.22 (d, J=5.0 Hz, 3H). MS (ESI) m/z 555.1 [M+H]⁺

Compound 146: General procedure A with variant iii) was used for the preparation from compound VI-A employing phenyl (3-methylisothiazol-5-yl)carbamate. ¹H NMR (400 MHz, DMSO-d₆) δ=10.98 (s, 1H), 10.38 (s, 1H), 7.65 (s, 1H), 7.59-7.53 (m, 2H), 7.30 (br t, J=5.7 Hz, 1H), 6.51 (s, 1H), 5.11 (dd, J=5.1, 13.3 Hz, 1H), 4.47-4.43 (m, 1H), 4.41 (s, 2H), 4.34-4.27 (m, 1H), 2.97-2.84 (m, 1H), 2.59 (br d, J=16.8 Hz, 1H), 2.46-2.34 (m, 1H), 2.24 (s, 3H), 2.05-1.95 (m, 1H). MS (ESI) m/z 414.1 [M+H]⁺

Preparation of phenyl (3-methylisothiazol-5-yl)carbamate: To a solution of 3-methylisothiazol-5-amine hydrochloride (0.260 g, 1.73 mmol, 1.30 eq, hydrochloride) in pyridine (2.00 mL) was added phenyl chloroformate (166 μL, 1.33 mmol, 1.00 eq). The reaction was stirred for 2 h at 0° C. The mixture was concentrated under reduced pressure to give a residue. The residue was washed with water to afford phenyl (3-methylisothiazol-5-yl)carbamate.

Compound 147: General procedure A with variant iii) was used for the preparation from compound VI-A employing phenyl(5-methylthiazol-2-yl)carbamate. ¹H NMR (400 MHz, DMSO-d₆) δ=11.00 (s, 1H), 10.38 (br s, 1H), 7.64 (s, 1H), 7.59-7.52 (m, 2H), 7.11 (br s, 1H), 6.96 (d, J=1.2 Hz, 1H), 5.12 (dd, J=5.1, 13.4 Hz, 1H), 4.48-4.24 (m, 4H), 2.97-2.84 (m, 1H), 2.69-2.55 (m, 1H), 2.45-2.31 (m, 1H), 2.27 (d, J=1.1 Hz, 3H), 2.05-1.94 (m, 1H). MS (ESI) m/z 414.1 [M+H]⁺

Preparation of phenyl(5-methylthiazol-2-yl)carbamate: To a mixture of 5-methylthiazol-2-amine (500 mg, 4.38 mmol, 1.00 eq) and pyridine (1.06 mL, 13.1 mmol, 3.00 eq) in dichloromethane (5.00 mL) was added phenyl chloroformate (575 μL, 4.60 mmol, 1.05 eq) dropwise at 0° C. over 20 min under nitrogen. The reaction was stirred at 0° C. for 2 h. The mixture was filtered, and the filtrate was concentrated under reduced pressure to afford phenyl (5-methylthiazol-2-yl)carbamate.

Compound 148: General procedure A with variant iii) was used for the preparation from compound VI-A employing phenyl (5-ethylthiazol-2-yl)carbamate. ¹H NMR (400 MHz, DMSO-d₆) δ=10.98 (s, 1H), 10.39 (br s, 1H), 7.65 (s, 1H), 7.56 (s, 2H), 7.13 (br s, 1H), 6.99 (s, 1H), 5.11 (dd, J=13, 5.2 Hz, 1H), 4.48-4.43 (m, 1H), 4.42 (d, J=5.8 Hz, 2H), 4.34-4.27 (m, 1H), 2.98-2.84 (m, 1H), 2.67 (q, J=7.4 Hz, 2H), 2.59 (br d, J=18 Hz, 1H), 2.40 (br d, J=13 Hz, 1H), 2.05-1.96 (m, 1H), 1.18 (t, J=7.6 Hz, 3H). MS (ESI) m/z 428.2 [M+H]⁺

Preparation of phenyl (5-ethylthiazol-2-yl)carbamate: To a solution of 5-ethylthiazol-2-amine (200 mg, 1.56 mmol, 1.00 eq) and pyridine (5.00 mL) in dichloromethane (10.0 mL) was added phenyl chloroformate (235 μL, 1.87 mmol, 1.20 eq) under nitrogen at 0° C. The reaction was stirred at 25° C. for 12 h. The mixture was extracted with ethyl acetate (3×10.0 mL). The combined organic layers were washed with brine (10.0 mL), dried over anhydrous sodium sulfate, filtered, and concentrated under reduced pressure to afford phenyl (5-ethylthiazol-2-yl)carbamate.

Compound 149: General procedure A with variant iii) was used for the preparation from compound VI-A employing phenyl (3-(trifluoromethyl)isothiazol-5-yl)carbamate. ¹H NMR (400 MHz, DMSO-d₆) δ=10.88-10.60 (m, 2H), 7.69 (s, 1H), 7.63-7.47 (m, 3H), 7.06 (s, 1H), 5.08 (dd, J=5.2, 13.1 Hz, 1H), 4.55-4.27 (m, 4H), 2.99-2.80 (m, 1H), 2.70-2.58 (m, 1H), 2.44-2.33 (m, 1H), 2.12-1.92 (m, 1H). MS (ESI) m/z 468.0 [M+H]⁺

### Preparation of phenyl (3-(trifluoromethyl)isothiazol-5-yl)carbamate

Step 1: To a solution of potassium tert-butoxide (1 M, 84.3 mL, 1.60 eq) at 0° C. was added a solution of ethyl 2,2,2-trifluoroacetate (7.27 mL, 52.7 mmol, 1.00 eq) in acetonitrile (3.19 mL, 60.6 mmol, 1.15 eq) dropwise at 0° C. The reaction was stirred at 20° C. for 24 hours. The reaction was quenched with hydrochloric acid (50.0 mL, 1 M) and extracted with ethyl acetate (3×50.0 mL). The organic phases were gathered, washed with brine (50.0 mL), dried over sodium sulfate, filtered, and concentrated under reduced pressure to afford 4,4,4-trifluoro-3-oxobutanenitrile.

Step 2: A mixture of 4,4,4-trifluoro-3-oxobutanenitrile (6.00 g, 43.8 mmol, 1.00 eq), ammonium formate (8.28 g, 131 mmol, 3.00 eq) and acetic acid (0.25 mL, 4.38 mmol, 0.100 eq) in toluene (10.0 mL) was heated to reflux in a Dean-Stark apparatus for 18 hours. The mixture was concentrated under reduced pressure to afford (Z)-3-amino-4,4,4-trifluorobut-2-enenitrile.

Step 3: To a solution of (Z)-3-amino-4,4,4-trifluorobut-2-enenitrile (6.00 g, 44.1 mmol, 1.00 eq) in dimethylformamide (30.0 mL) was added magnesium chloride (1.81 mL, 44.1 mmol, 1.00 eq) and sodium hydrogen sulfide (4.94 g, 88.2 mmol, 2.00 eq) in portions. The reaction was stirred at 25° C. for 18 hours. The mixture was diluted with water (10.0 mL) and extracted with ethyl acetate (5×20.0 mL). The organic phases were gathered, washed with brine (20.0 mL), dried over sodium sulfate, filtered, and concentrated under reduced pressure to afford (Z)-3-amino-4,4,4-trifluoro-but-2-enethioamide.

Step 4: To an ice-cold mixture of (Z)-3-amino-4,4,4-trifluoro-but-2-enethioamide (1.50 g, 8.82 mmol, 1.00 eq) in pyridine (10.0 mL) was added hydrogen peroxide 30% purity (1.69 mL, 17.6 mmol, 2.00 eq) at 0° C. The mixture was stirred at 25° C. for 2 hours. The mixture was dried under nitrogen to give a residue. The residue was diluted with aqueous sodium sulfite solution (20.0 mL) and extracted with ethyl acetate (3×20.0 mL). The combined organic layers were washed with saturated aqueous citric acid solution (2×20.0 mL) and brine (20.0 mL), dried over sodium sulfate, filtered, and concentrated under reduced pressure to give a residue. The residue was purified by silica gel column chromatography (petroleum ether/ethyl acetate=5/1 to 1/1) to afford 3-(trifluoromethyl)isothiazol-5-amine.

Step 5: To a solution of 3-(trifluoromethyl)isothiazol-5-amine (250 mg, 1.49 mmol, 1.00 eq) in pyridine (2.00 mL) was added phenyl chloroformate (0.28 mL, 2.23 mmol, 1.50 eq) dropwise at 0° C. The reaction was stirred for 4 hours at 25° C. The mixture was diluted with water (5.00 mL) and extracted with ethyl acetate (2×10.0 mL). The organic phases were gathered, washed with brine (10.0 mL), dried over sodium sulfate, filtered, and concentrated and the obtained residue was purified by standard methods to afford phenyl (3-(trifluoromethyl)isothiazol-5-yl)carbamate.

Compound 150: General procedure A with variant iii) was used for the preparation from compound VI-A employing phenyl (4,5-dimethylthiazol-2-yl)carbamate. ¹H NMR (400 MHz, DMSO-d₆) δ=11.00 (s, 1H), 10.29 (br s, 1H), 7.64 (s, 1H), 7.60-7.52 (m, 2H), 7.13 (br s, 1H), 5.12 (dd, J=5.0, 13.2 Hz, 1H), 4.48-4.43 (m, 1H), 4.41 (s, 2H), 4.35-4.26 (m, 1H), 2.98-2.85 (m, 1H), 2.59 (br d, J=17.3 Hz, 1H), 2.43-2.35 (m, 1H), 2.17 (s, 3H), 2.08 (s, 3H), 2.04-1.94 (m, 1H). MS (ESI) m/z 428.1 [M+H]⁺

Preparation of phenyl (4,5-dimethylthiazol-2-yl)carbamate: Phenyl chloroformate (0.20 mL, 1.64 mmol, 1.05 eq) was added dropwise to solution of 4,5-dimethylthiazol-2-amine (0.200 g, 1.56 mmol, 1.00 eq) and pyridine (0.38 mL, 4.68 mmol, 3.00 eq) in dichloromethane (2.00 mL) at 0° C. The reaction was stirred at 0° C. for 2 h. Additional phenyl chloroformate (49.0 μL, 390 μmol, 0.25 eq) was added, and the reaction was stirred at 0° C. for 1 h. Water (2.00 mL) was added slowly over 30 min, and the mixture was diluted with dichloromethane (10 mL). The organic layer was washed with saturated aqueous sodium carbonate (3.00 mL) and brine (2.00 mL), dried over sodium sulfate, filtered, and concentrated and the obtained residue was purified by standard methods to afford phenyl (4,5-dimethylthiazol-2-yl)carbamate.

Compound 151: General procedure A with variant iii) was used for the preparation from compound VI-A employing phenyl (4-methyl-3-(morpholinomethyl)-5-(trifluoromethoxy)phenyl)carbamate. ¹H NMR (400 MHz, DMSO-d₆) δ=10.99 (s, 1H), 8.91 (s, 1H), 8.16 (s, 1H), 7.70-7.64 (m, 1H), 7.64-7.60 (m, 1H), 7.60-7.52 (m, 2H), 7.17 (d, J=2.2 Hz, 1H), 6.79 (t, J=6.0 Hz, 1H), 5.11 (dd, J=5.0, 13.3 Hz, 1H), 4.49-4.25 (m, 4H), 3.56 (br t, J=4.3 Hz, 4H), 3.40 (s, 2H), 2.96-2.85 (m, 1H), 2.63-2.56 (m, 1H), 2.46-2.33 (m, 5H), 2.16 (s, 3H), 2.03-1.96 (m, 1H). MS (ESI) m/z 590.3 [M+H]⁺

### Preparation of phenyl (4-methyl-3-(morpholinomethyl)-5-(trifluoromethoxy)phenyl)carbamate

Step 1: To a solution of 2-methyl-5-nitro-benzoic acid (10.0 g, 55.2 mmol, 1.00 eq) in sulfuric acid (20.0 mL) was added N-Iodosuccinimide (14.9 g, 66.3 mmol, 1.20 eq). The reaction was stirred at 60° C. for 2 h. The mixture was diluted with ice water (200 mL) and filtered. The filter cake was washed with water (100 mL) and dried under vacuum to afford 3-iodo-2-methyl-5-nitro-benzoic acid.

Step 2: To a solution of 3-iodo-2-methyl-5-nitro-benzoic acid (5.00 g, 16.3 mmol, 1.00 eq), copper iodide (310 mg, 1.63 mmol, 0.10 eq) and quinolin-8-ol (563 μL, 3.26 mmol, 0.20 eq) in water (3.00 mL) and dimethylsulfoxide (3.00 mL) was added a solution of potassium hydroxide (3.65 g, 65.1 mmol, 4.00 eq). The reaction was stirred at 100° C. for 12 h. The mixture was diluted with water (50.0 mL) and extracted with ethyl acetate (2×50.0 mL). The combined organic layers were washed with water (50.0 mL), dried over anhydrous sodium sulfate, filtered, and concentrated under reduced pressure to afford 3-hydroxy-2-methyl-5-nitro-benzoic acid.

Step 3: To a solution of 3-hydroxy-2-methyl-5-nitro-benzoic acid (3.20 g, 16.2 mmol, 1.00 eq) and morpholine (1.71 mL, 19.5 mmol, 1.20 eq) in dichloromethane (100 mL) was added triethylamine (2.26 mL, 16.2 mmol, 1.00 eq) and O-(7-azabenzotriazol-1-yl)-N,N,N,N-tetramethyluroniumhexafluorophosphate (7.41 g, 19.5 mmol, 1.20 eq) at 20° C. The reaction was stirred at 20° C. for 12 h. The mixture was concentrated under reduced pressure to give a residue. The residue was diluted with water (100 mL) and extracted with ethyl acetate (100 mL). The organic layer was washed with water (100 mL), dried over anhydrous sodium sulfate, filtered, and the filtrate was concentrated under reduced pressure to give a residue. The residue was purified by silica gel column chromatography (petroleum ether/ethyl acetate=1/1 to 0/1) to afford (3-hydroxy-2-methyl-5-nitro-phenyl)-morpholino-methanone.

Step 4: To a solution of (3-hydroxy-2-methyl-5-nitro-phenyl)-morpholino-methanone (1.30 g, 4.88 mmol, 1.00 eq), silver trifluoromethanesulfonate (6.27 g, 24.4 mmol, 5.00 eq), 1-(chloromethyl)-4-fluoro-1,4-diazoniabicyclo[2.2.2]octane bis(tetrafluoroborate) (3.46 g, 9.77 mmol, 2.00 eq), N-fluorobenzenesulfonimide (3.08 g, 9.77 mmol, 2.00 eq) and caesium fluoride (4.45 g, 29.3 mmol, 1.08 mL, 6.00 eq) in toluene (130 mL) was added trimethyl(trifluoromethyl)silane (3.47 g, 24.4 mmol, 5.00 eq) and 2-fluoropyridine (2.10 mL, 24.4 mmol, 5.00 eq) under nitrogen. The reaction was stirred at 20° C. for 12 h under nitrogen. The mixture was filtered, and the filtrate was concentrated under reduced pressure to afford a residue. The residue was diluted with water (100 mL) and extracted with ethyl acetate (100 mL). The organic layer was washed with water (20.0 mL), dried over anhydrous sodium sulfate, filtered, and the filtrate was concentrated under reduced pressure to give a residue. The residue was purified by silica gel column chromatography (petroleum ether/ethyl acetate=1/0 to 2/1) to afford (2-methyl-5-nitro-3-(trifluoromethoxy)phenyl)(morpholino)methanone.

Step 5: To a solution of (2-methyl-5-nitro-3-(trifluoromethoxy)phenyl)-morpholino-methanone (900 mg, 2.69 mmol, 1.00 eq) in tetrahydrofuran (15.0 mL) was added borane dimethyl sulfide complex (10.0 M, 539 μL, 2.00 eq) at 0° C. The reaction was stirred at 60° C. for 30 min. The mixture was quenched with methanol (2.00 mL) and concentrated under reduced pressure to afford a residue. The residue was purified by reversed phase column chromatography to afford 4-(2-methyl-5-nitro-3-(trifluoromethoxy) benzyl)morpholine.

Step 6: To a solution of 4-(2-methyl-5-nitro-3-(trifluoromethoxy)benzyl)morpholine (400 mg, 1.25 mmol, 1.00 eq) in methanol (5.00 mL) and water (5.00 mL) was added iron powder (488 mg, 8.74 mmol, 7.00 eq) and ammonium chloride (468 mg, 8.74 mmol, 7.00 eq). The reaction was stirred at 80° C. for 2 h. The mixture was diluted with saturated sodium carbonate (1.00 mL) and extracted with ethyl acetate (2×10.0 mL). The combined organic layers were washed with water (5.00 mL), dried over anhydrous sodium sulfate, filtered, and the filtrate was concentrated under reduced pressure to afford a residue. The residue was purified by silica gel column chromatography (petroleum ether/ethyl acetate=3/1 to 1/1) to afford 4-methyl-3-(morpholinomethyl)-5-(trifluoromethoxy)aniline.

Step 7: To a solution of 4-methyl-3-(morpholinomethyl)-5-(trifluoromethoxy)aniline (100 mg, 344 μmol, 1.00 eq) and potassium carbonate (57.1 mg, 413 μmol, 1.20 eq) in acetone (1.00 mL) was added phenyl chloroformate (47 μL, 379 μ, 1.10 eq) at 25° C. The reaction was stirred at 25° C. for 1 h. The mixture was diluted with water (6.00 mL) and extracted with ethyl acetate (10.0 mL). The organic layer was dried over anhydrous sodium sulfate, filtered, and the filtrate was concentrated to afford phenyl (4-methyl-3-(morpholinomethyl)-5-(trifluoromethoxy)phenyl)carbamate.

Compound 152: General procedure A with variant iii) was used for the preparation from compound VI-A employing phenyl 3-chloro-4-methylbenzylcarbamate. ¹H NMR (400 MHz, DMSO-d₆) δ=10.98 (br s, 1H), 8.49 (s, 1H), 7.63 (s, 1H), 7.56-7.48 (m, 2H), 7.29-7.23 (m, 2H), 7.13-7.08 (m, 1H), 6.68-6.53 (m, 2H), 5.15-5.07 (m, 1H), 4.46-4.38 (m, 1H), 4.35-4.26 (m, 3H), 4.19 (d, J=6.0 Hz, 2H), 2.98-2.84 (m, 1H), 2.63-2.58 (m, 1H), 2.43-2.31 (m, 1H), 2.28 (s, 3H), 2.05-1.93 (m, 1H). MS (ESI) m/z 455.2 [M+H]⁺

Preparation of phenyl 3-chloro-4-methylbenzylcarbamate: To a solution of (3-chloro-4-methyl-phenyl)methanamine (500 mg, 3.21 mmol, 1.00 eq) and pyridine (0.78 mL, 9.64 mmol, 3.00 eq) in acetonitrile (5.00 mL) was added phenyl chloroformate (0.60 mL, 4.82 mmol, 1.50 eq). The reaction was stirred at 25° C. for 12 h. The mixture was concentrated under reduced pressure to give a residue. The residue was diluted with ethyl acetate (30.0 mL) and water (50.0 mL). The organic layer was separated, and the aqueous phase was extracted with ethyl acetate (3×30.0 mL). The organic layers were gathered, dried over sodium sulfate, filtered, and concentrated and the obtained residue was purified by standard methods to afford phenyl 3-chloro-4-methylbenzylcarbamate.

Compound 153: General procedure A with variant iii) was used for the preparation from compound VI-A employing phenyl benzo[d][1,3]dioxol-5-ylcarbamate. ¹H NMR (400 MHz, DMSO-d₆) δ=10.97 (s, 1H), 8.49 (s, 1H), 7.65 (s, 1H), 7.55 (d, J=1.0 Hz, 2H), 7.17 (d, J=2.0 Hz, 1H), 6.80-6.74 (m, 1H), 6.72-6.68 (m, 1H), 6.65 (t, J=6.0 Hz, 1H), 5.93 (s, 2H), 5.14-5.08 (m, 1H), 4.47-4.27 (m, 4H), 2.97-2.84 (m, 1H), 2.64-2.56 (m, 1H), 2.43-2.31 (m, 1H), 2.05-1.95 (m, 1H). MS (ESI) m/z 437.3[M+H]⁺

Preparation of phenyl benzo[d][1,3]dioxol-5-ylcarbamate: To a solution of benzo[d][1,3]dioxol-5-amine (1.00 g, 7.29 mmol, 1.00 eq) and pyridine (1.77 mL, 21.9 mmol, 3.00 eq) in acetonitrile (10 mL) was added phenyl chloroformate (1.37 mL, 11.0 mmol, 1.50 eq). The reaction was stirred at 25° C. for 12 h. The mixture was concentrated under reduced pressure to give a residue. The residue was diluted with ethyl acetate (30 mL) and water (50 mL). The organic layer was separated, and the aqueous layer was extracted with ethyl acetate (3×30 mL). The combined organic layers were dried over sodium sulfate, filtered, and concentrated under reduced pressure to give a residue. The residue was purified by silica gel column chromatography (petroleum ether/ethyl acetate=3/1) to afford phenyl benzo[d][1,3]dioxol-5-ylcarbamate.

Compound 154: General procedure A with variant iii) was used for the preparation from compound VI-A employing phenyl (3-(1,4-diazabicyclo[3.2.1]octan-4-ylmethyl)-5-chloro-4-methylphenyl) carbamate. ¹H NMR (400 MHz, DMSO-d₆) δ=10.99 (br s, 1H), 8.95 (s, 1H), 8.28 (s, 1H), 7.66 (s, 1H), 7.62 (d, J=2.2 Hz, 1H), 7.56 (d, J=0.7 Hz, 2H), 7.15 (s, 1H), 7.08-6.96 (m, 1H), 5.12 (dd, J=5.1, 13.3 Hz, 1H), 4.47-4.27 (m, 4H), 3.40-3.35 (m, 1H), 3.30-3.27 (m, 1H), 3.18-3.14 (m, 1H), 2.96-2.81 (m, 5H), 2.67-2.53 (m, 4H), 2.46-2.35 (m, 2H), 2.26 (s, 3H), 2.11-1.95 (m, 2H), 1.60-1.46 (m, 1H). MS (ESI) m/z 565.3 [M+H]⁺

### Preparation of phenyl (3-(1,4-diazabicyclo[3.2.1]octan-4-ylmethyl)-5-chloro-4-methylphenyl) carbamate

Step 1: To a solution of 2-methyl-5-nitro-benzoic acid (5.00 g, 27.6 mmol, 1.00 eq) in sulfuric acid (10.0 mL) was added 1,3-dichloro-5,5-dimethyl-imidazolidine-2,4-dione (6.53 g, 33.1 mmol, 1.20 eq) dropwise at 80° C. The reaction was stirred at 80° C. for 12 h. The mixture was poured into ice water (100 mL) and filtered. The filter cake was dissolved into ethyl acetate (200 mL), and the organic layer was washed with water (100 mL), dried over anhydrous sodium sulfate, filtered, and concentrated under reduced pressure to afford 3-chloro-2-methyl-5-nitro-benzoic acid.

Step 2: To a solution of 3-chloro-2-methyl-5-nitro-benzoic acid (388 mg, 1.80 mmol, 1.00 eq) and 1,4-diazabicyclo[3.2.1]octane dihydrochloride (300 mg, 1.62 mmol, 0.90 eq, dihydrochloride) in dichloromethane (5.00 mL) was added triethylamine (0.75 mL, 5.40 mmol, 3.00 eq) and O-(7-azabenzotriazol-1-yl)-N,N,N,N-tetramethyluroniumhexafluorophosphate (822 mg, 2.16 mmol, 1.20 eq) at 20° C. The reaction was stirred at 20° C. for 2 h. The mixture was concentrated under reduced pressure to afford a residue. The residue was diluted with water (20.0 mL) and saturated aqueous sodium carbonate (3.00 mL), then extracted with ethyl acetate (50.0 mL). The organic layer was washed with water (10.0 mL), dried over anhydrous sodium sulfate, filtered, and the filtrate was concentrated under reduced pressure to afford (3-chloro-2-methyl-5-nitro-phenyl)-(1,4-diazabicyclo[3.2.1]octan-4-yl)methanone.

Step 3: To a solution of (3-chloro-2-methyl-5-nitro-phenyl)-(1,4-diazabicyclo[3.2.1]octan-4-yl)methanone (700 mg, 2.26 mmol, 1.00 eq) in tetrahydrofuran (20.0 mL) was added borane dimethyl sulfide complex (10.0 M, 0.45 mL, 2.00 eq) at 0° C. The reaction was stirred at 60° C. for 30 min. The mixture was quenched with methanol (0.500 mL) and concentrated under reduced pressure to give a residue. The residue was diluted with water (10.0 mL) and saturated aqueous sodium carbonate (10.0 mL), then extracted with ethyl acetate (30.0 mL). The organic layer was washed with water (10.0 mL), dried over anhydrous sodium sulfate, filtered, and the filtrate was concentrated under reduced pressure to give a residue. The residue was purified by silica gel column chromatography (petroleum ether/ethyl acetate=1/0 to 2/1) to afford 4-(3-chloro-2-methyl-5-nitrobenzyl)-1,4-diazabicyclo [3.2.1]octane.

Step 4: To a solution of 4-(3-chloro-2-methyl-5-nitrobenzyl)-1,4-diazabicyclo[3.2.1]octane (150 mg, 507 μmol, 1.00 eq) in methanol (1.50 mL) and water (1.50 mL) was added iron powder (198 mg, 3.55 mmol, 7.00 eq) and ammonium chloride (190 mg, 3.55 mmol, 7.00 eq). The reaction was stirred at 80° C. for 2 h. The mixture was diluted with saturated aqueous sodium carbonate (1.00 mL) and extracted with ethyl acetate (2×10.0 mL). The organic layers were washed with water (5.00 mL), dried over anhydrous sodium sulfate, filtered, and the filtrate was concentrated under reduced pressure to afford 3-(1,4-diazabicyclo[3.2.1]octan-4-ylmethyl)-5-chloro-4-methylaniline.

Step 5: To a solution of 3-(1,4-diazabicyclo[3.2.1]octan-4-ylmethyl)-5-chloro-4-methylaniline (100 mg, 376 μmol, 1.00 eq) and potassium carbonate (62.4 mg, 452 μmol, 1.20 eq) in acetone (2.00 mL) was added phenyl chloroformate (51.8 μL, 414 μmol, 1.10 eq) at 25° C. The reaction was stirred at 25° C. for 1 h. The mixture was diluted with water (3.00 mL) and extracted with ethyl acetate (5.00 mL). The organic layer was dried over anhydrous sodium sulfate, filtered, and the filtrate was concentrated to afford phenyl (3-(1,4-diazabicyclo[3.2.1] octan-4-ylmethyl)-5-chloro-4-methylphenyl)carbamate.

Compound 155: General procedure A with variant iii) was used for the preparation from compound VI-A employing phenyl (3-chloro-4-methyl-5-(3-methyl-3-azabicyclo[3.1.0]hexan-1-yl)phenyl)carbamate. ¹H NMR (400 MHz, DMSO-d₆) δ=10.99 (br s, 1H), 9.02 (s, 1H), 8.31 (s, 1H), 7.71-7.50 (m, 4H), 7.19 (d, J=2.0 Hz, 1H), 7.06 (br s, 1H), 5.12 (dd, J=5.2, 13.2 Hz, 1H), 4.52-4.25 (m, 4H), 3.12-2.85 (m, 4H), 2.63-2.57 (m, 1H), 2.40 (br dd, J=4.2, 13.2 Hz, 1H), 2.31 (s, 3H), 2.26 (s, 3H), 2.19 (d, J=8.6 Hz, 1H), 2.06-1.94 (m, 1H), 1.63 (td, J=3.6, 7.6 Hz, 1H), 1.32-1.13 (m, 1H), 0.59 (dd, J=3.6, 8.0 Hz, 1H). MS (ESI) m/z 536.3 [M+H]⁺

### Preparation of phenyl (3-chloro-4-methyl-5-(3-methyl-3-azabicyclo[3.1.0]hexan-1-yl)phenyl)carbamate

Step 1: A solution of 1-(bromomethyl)-3-chloro-2-methyl-5-nitrobenzene (25.0 g, 94.5 mmol, 1.00 eq) and sodium cyanide (6.02 g, 123 mmol, 1.30 eq) in acetonitrile (270 mL) and water (36.0 mL) was stirred at 80° C. for 10 h under nitrogen. The reaction was quenched by addition of saturated aqueous sodium carbonate (60.0 mL), and the mixture was extracted with ethyl acetate (3×200 mL). The combined organic layers were washed with brine (3×100 mL), dried over sodium sulfate, filtered, and concentrated under reduced pressure to give a residue. The residue was purified by silica gel column chromatography (petroleum ether/ethyl acetate=0/1 to 1/4) to afford 2-(3-chloro-2-methyl-5-nitrophenyl)acetonitrile.

Step 2: A mixture of 2-(3-chloro-2-methyl-5-nitrophenyl)acetonitrile (17.9 g, 85.0 mmol, 1.00 eq), ferrous powder (23.7 g, 425 mmol, 5.00 eq) and ammonium chloride (4.55 g, 85.0 mmol, 1.00 eq) in ethanol (160 mL) and water (80.0 mL) was stirred at 60° C. for 10 h. The mixture was concentrated under reduced pressure to give a residue. The residue was diluted with water (80.0 mL) and extracted with ethyl acetate (3×150 mL). The combined organic layers were washed with brine (3×50.0 mL), dried over sodium sulfate, filtered, and concentrated under reduced pressure to give a residue. The residue was purified by silica gel column chromatography (petroleum ether/ethyl acetate=3/1) to afford 2-(5-amino-3-chloro-2-methylphenyl)acetonitrile.

Step 3: A mixture of 2-(5-amino-3-chloro-2-methylphenyl)acetonitrile (14.7 g, 81.4 mmol, 1.00 eq), hexane-2,5-dione (9.29 g, 81.4 mmol, 9.55 mL, 1.00 eq) and p-toluenesulfonic acid monohydrate (0.16 g, 841 μmol, 0.002 eq) in toluene (150 mL) was stirred at 110° C. for 10 h. The mixture was concentrated under reduced pressure to give a residue. The residue was purified by silica gel column chromatography (petroleum ether/ethyl acetate=5/1) to afford 2-(3-chloro-5-(2,5-dimethyl-1H-pyrrol-1-yl)-2-methylphenyl)acetonitrile.

Step 4: To a solution of sodium hexamethyldisilazane (1.00 M, 34.8 mL, 1.80 eq) in tetrahydrofuran (50.0 mL) was added 2-(3-chloro-5-(2,5-dimethyl-1H-pyrrol-1-yl)-2-methylphenyl)acetonitrile (5.00 g, 19.3 mmol, 1.00 eq) at −10° C. After 12 mins, 2-(chloromethyl)oxirane (2.23 g, 24.2 mmol, 1.89 mL, 1.25 eq) was added, and the reaction was stirred at 25° C. for 12 h. Water (20.0 mL) was added to quench the reaction, and the mixture was extracted with ethyl acetate (3×50.0 mL). The combined organic layers were washed with brine (20.0 mL), dried over sodium sulfate, filtered, and concentrated under reduced pressure to give a residue. The residue was purified by silica gel column chromatography (petroleum ether/ethyl acetate=1/1) to afford 1-(3-chloro-5-(2,5-dimethyl-1H-pyrrol-1-yl)-2-methylphenyl)-2-(hydroxymethyl)cyclopropanecarbonitrile.

Step 5: To a solution of 1-(3-chloro-5-(2,5-dimethyl-1H-pyrrol-1-yl)-2-methylphenyl)-2-(hydroxymethyl) cyclopropanecarbonitrile (3.50 g, 11.1 mmol, 1.00 eq) in tetrahydrofuran (40.0 mL) was added borane dimethyl sulfide complex (10.0 M, 3.34 mL, 3.00 eq) at 0° C. The reaction was stirred at 50° C. for 0.5 h. The reaction was quenched by addition methanol (35.0 mL) and concentrated under reduced pressure to afford (2-(aminomethyl)-2-(3-chloro-5-(2,5-dimethyl-1H-pyrrol-1-yl)-2-methylphenyl) cyclopropyl)methanol.

Step 6: To a solution of (2-(aminomethyl)-2-(3-chloro-5-(2,5-dimethyl-1H-pyrrol-1-yl)-2-methylphenyl)cyclopropyl)methanol (3.50 g, 11.0 mmol, 1.00 eq) and triphenylphosphine (3.46 g, 13.2 mmol, 1.20 eq) in tetrahydrofuran (35.0 mL) was added diisopropyl azodicarboxylate (2.56 mL, 13.2 mmol, 1.20 eq) at 0° C. The reaction was stirred at 25° C. for 10 h. The mixture was concentrated under reduced pressure to give a residue. The residue was purified twice by reversed phase column chromatography and lyophilized to afford 1-(3-chloro-5-(2,5-dimethyl-1H-pyrrol-1-yl)-2-methylphenyl)-3-azabicyclo[3.1.0]hexane.

Step 7: To a solution of 1-(3-chloro-5-(2,5-dimethyl-1H-pyrrol-1-yl)-2-methylphenyl)-3-azabicyclo[3.1.0]hexane (0.20 g, 577 μmol, 1.00 eq), sodium cyanoborohydride (109 mg, 1.73 mmol, 3.00 eq) and formaldehyde 37% purity (1.67 mL, 22.4 mmol, 38.8 eq) in methanol (5.00 mL) was added acetic acid (33.0 μL, 577 μmol, 1.00 eq). The reaction was stirred at 25° C. for 2 h. The mixture was diluted with water (10.0 mL) and extracted with dichloromethane (3×30.0 mL). The combined organic layers were washed with brine (10.0 mL), dried over sodium sulfate, filtered, and concentrated under reduced pressure to afford 1-(3-chloro-5-(2,5-dimethyl-1H-pyrrol-1-yl)-2-methylphenyl)-3-methyl-3-azabicyclo[3.1.0]hexane.

Step 8: A solution of 1-(3-chloro-5-(2,5-dimethyl-1H-pyrrol-1-yl)-2-methylphenyl)-3-methyl-3-azabicyclo[3.1.0] hexane (0.10 g, 318 μmol, 1.00 eq) and hydroxylamine hydrochloride (221 mg, 3.18 mmol, 10.0 eq) in ethanol (1.00 mL) and water (0.50 mL) was stirred at 100° C. for 12 h. The mixture was concentrated under reduced pressure to give a residue. The residue was purified was by reversed phase column chromatography and lyophilized to afford 3-chloro-4-methyl-5-(3-methyl-3-azabicyclo[3.1.0]hexan-1-yl)aniline.

Step 9: To a solution of 3-chloro-4-methyl-5-(3-methyl-3-azabicyclo[3.1.0]hexan-1-yl)aniline (50.0 mg, 211 μmol, 1.00 eq) and potassium carbonate (58.4 mg, 422 μmol, 2.00 eq) in acetone (1.00 mL) was added phenyl chloroformate (31.7 μL, 253 μmol, 1.20 eq). The reaction was stirred at 25° C. for 10 h. The mixture was diluted with water (8.00 mL) and extracted with ethyl acetate (3×30.0 mL). The combined organic layers were washed with brine (10.0 mL), dried over sodium sulfate, filtered, and concentrated under reduced pressure to afford phenyl (3-chloro-4-methyl-5-(3-methyl-3-azabicyclo[3.1.0] hexan-1-yl)phenyl)carbamate.

### Compound 156:

Step 1: To a solution of (3-chloro-2-methyl-5-nitro-phenyl)methanol (1.00 g, 4.96 mmol, 1.00 eq) and imidazole (675 mg, 9.92 mmol, 2.00 eq) in dichloromethane (10.0 mL) was added tert-butyldimethylsilyl chloride (0.73 mL, 5.95 mmol, 1.20 eq) at 0° C. The reaction was stirred at 25° C. for 1 h. The mixture was concentrated under reduced pressure to give a residue. The residue was purified by silica gel column chromatography (petroleum ether/ethyl acetate=1/0 to 50/1) to afford tert-butyl((3-chloro-2-methyl-5-nitrobenzyl)oxy)dimethylsilane.

Step 2: A mixture of tert-butyl((3-chloro-2-methyl-5-nitrobenzyl)oxy)dimethylsilane (1.50 g, 4.75 mmol, 1.00 eq), iron powder (795 mg, 14.2 mmol, 3.00 eq) and ammonium chloride (1.27 g, 23.7 mmol, 5.00 eq) in methanol (10.0 mL) and water (5.00 mL) was stirred at 80° C. for 1 h. The mixture was filtered, and the filtrate was concentrated under reduced pressure to give a residue. The residue was added to water (100 mL) and stirred for 10 min. The mixture was extracted with ethyl acetate (3×60.0 mL). The combined organic phases were washed with brine (100 mL), dried over anhydrous sodium sulfate, filtered, and concentrated under reduced pressure to afford 3-(((tert-butyldimethylsilyl)oxy)methyl)-5-chloro-4-methylaniline.

Step 3: To a solution of 3-(((tert-butyldimethylsilyl)oxy)methyl)-5-chloro-4-methylaniline (900 mg, 3.15 mmol, 1.00 eq) and pyridine (0.76 mL, 9.44 mmol, 3.00 eq) in acetonitrile (10.0 mL) was added phenyl chloroformate (0.43 mL, 3.46 mmol, 1.10 eq) at 0° C. The reaction was stirred at 25° C. for 1 h. The mixture was concentrated to give a residue. Water (100 mL) was added, and the mixture was stirred for 10 min. The mixture was extracted with ethyl acetate (3×60.0 mL). The combined organic layers were washed with brine (100 mL), dried with anhydrous sodium sulfate, filtered, and concentrated under reduced pressure to give a residue. The residue was purified by silica gel column chromatography (petroleum ether/ethyl acetate=1/0 to 10/1) to afford phenyl (3-(((tert-butyldimethylsilyl)oxy) methyl)-5-chloro-4-methylphenyl)carbamate.

Step 4: To a solution of 3-(6-(aminomethyl)-1-oxoisoindolin-2-yl)piperidine-2,6-dione hydrochloride VI-A (359 mg, 1.31 mmol, 1.00 eq, hydrochloride) and triethylamine (0.73 mL, 5.25 mmol, 4.00 eq) in dimethylformamide (5.00 mL) was added phenyl (3-(((tert-butyldimethylsilyl)oxy)methyl)-5-chloro-4-methylphenyl)carbamate (800 mg, 1.97 mmol, 1.50 eq). The reaction was stirred at 25° C. for 1 h. The mixture was filtered, and the filtrate was concentrated under reduced pressure to give a residue. The residue was purified by reversed phase preparative HPLC to afford 1-(3-(((tert-butyldimethylsilyl)oxy)methyl)-5-chloro-4-methylphenyl)-3-((2-(2,6-dioxopiperidin-3-yl)-3-oxoisoindolin-5-yl)methyl)urea.

Step 5: To a solution of 1-(3-(((tert-butyldimethylsilyl)oxy)methyl)-5-chloro-4-methylphenyl)-3-((2-(2,6-dioxopiperidin-3-yl)-3-oxoisoindolin-5-yl)methyl)urea (350 mg, 598 μmol, 1.00 eq) in tetrahydrofuran (5.00 mL) was added tetrabutylammonium fluoride trihydrate (283 mg, 897 μmol, 1.50 eq). The reaction was stirred at 25° C. for 2 h. Saturated aqueous ammonium chloride (20.0 mL) was added to quench the reaction, and the mixture was poured into water (60.0 mL) and stirred for 10 min. The aqueous layer was extracted with ethyl acetate (3×50.0 mL). The combined organic layers were washed with brine (100 mL), dried over anhydrous sodium sulfate, filtered, and concentrated under reduced pressure to give a residue. The residue was purified by reversed phase preparative HPLC to afford 1-(3-chloro-5-(hydroxymethyl)-4-methylphenyl)-3-((2-(2,6-dioxopiperidin-3-yl)-3-oxoisoindolin-5-yl)methyl)urea.

Step 6: To a solution of 1-(3-chloro-5-(hydroxymethyl)-4-methylphenyl)-3-((2-(2,6-dioxopiperidin-3-yl)-3-oxoisoindolin-5-yl)methyl)urea (150 mg, 318° μmol, 1.00 eq) in dichloromethane (1.00 mL) was added manganese dioxide (110 mg, 1.27 mmol, 4.00 eq). The reaction was stirred at 25° C. for 12 h. The mixture was filtered, and the filtrate was concentrated and the obtained residue was purified by standard methods to afford Compound 156.

¹H NMR (400 MHz, DMSO-d₆) δ=10.98 (s, 1H), 10.20 (s, 1H), 9.21 (s, 1H), 7.94 (d, J=2.3 Hz, 1H), 7.78 (d, J=2.4 Hz, 1H), 7.70-7.64 (m, 1H), 7.62-7.52 (m, 2H), 7.16-7.02 (m, 1H), 5.12 (dd, J=5.1, 13.3 Hz, 1H), 4.52-4.36 (m, 3H), 4.36 (br s, 1H), 2.97-2.82 (m, 1H), 2.69-2.60 (m, 1H), 2.58-2.55 (m, 3H), 2.47-2.33 (m, 1H), 2.06-1.96 (m, 1H). MS (ESI) m/z 469.2 [M+H]⁺

Compound 157: General procedure A with variant iii) was used for the preparation from compound VI-A employing phenyl (5-chloro-6-methylpyridin-3-yl)carbamate. ¹H NMR (400 MHz, DMSO-d₆) δ=10.98 (s, 1H), 9.83 (s, 1H), 8.60 (d, J=2.2 Hz, 1H), 8.27 (d, J=2.2 Hz, 1H), 7.66 (s, 1H), 7.56 (s, 2H), 7.40 (br s, 1H), 5.16-5.06 (m, 1H), 4.41-4.27 (m, 4H), 2.96-2.84 (m, 1H), 2.59 (br d, J=18.2 Hz, 1H), 2.54 (s, 3H), 2.43-2.33 (m, 1H), 2.04-1.94 (m, 1H). MS (ESI) m/z 442.2 [M+H]⁺

### Preparation of phenyl (5-chloro-6-methylpyridin-3-yl)carbamate

Step 1: A mixture of 3-chloro-2-methyl-5-nitropyridine (500 mg, 2.90 mmol, 1.00 eq), iron powder (1.13 g, 20.3 mmol, 7.00 eq) and ammonium chloride (1.08 g, 20.3 mmol, 7.00 eq) in methanol (5.00 mL) and water (5.00 mL) was stirred at 80° C. for 2 h. The mixture was filtered over Celite, and the filtrate was concentrated under reduced pressure to afford 5-chloro-6-methylpyridin-3-amine.

Step 2: To a solution of 5-chloro-6-methylpyridin-3-amine (200 mg, 1.40 mmol, 1.00 eq) and pyridine (0.34 mL, 4.21 mmol, 3.00 eq) in acetonitrile (2.00 mL) was added phenyl chloroformate (264 μL, 2.10 mmol, 1.50 eq). The reaction was stirred at 25° C. for 12 h. The mixture was concentrated under reduced pressure to give a residue. Ethyl acetate (30.0 mL) and water (50.0 mL) were added, and the organic layer was separated. The aqueous phase was extracted with ethyl acetate (3×100 mL). The combined organic layers were dried over sodium sulfate, filtered, and concentrated and the obtained residue was purified by standard methods to afford phenyl (5-chloro-6-methylpyridin-3-yl)carbamate.

Compound 158: General procedure A with variant iii) was used for the preparation from compound VI-A employing phenyl (2,6-dimethylpyridin-4-yl)carbamate. ¹H NMR (400 MHz, DMSO-d₆) δ=10.99 (s, 1H), 9.16 (br s, 1H), 8.19 (s, 1H), 7.66 (s, 1H), 7.57 (s, 2H), 7.16 (br t, J=5.7 Hz, 1H), 7.11 (s, 2H), 5.12 (dd, J=5.1, 13.3 Hz, 1H), 4.45-4.31 (m, 4H), 2.98-2.85 (m, 1H), 2.64-2.56 (m, 1H), 2.46-2.37 (m, 1H), 2.32 (s, 6H), 2.05-1.95 (m, 1H). MS (ESI) m/z 422.1 [M+H]⁺

Preparation of phenyl (2,6-dimethylpyridin-4-yl)carbamate: To a solution of 2,6-dimethylpyridin-4-amine (1.00 g, 8.19 mmol, 1.00 eq) in acetonitrile (20.0 mL) was added pyridine (3.30 mL, 40.9 mmol, 5.00 eq) and phenyl chloroformate (1.54 mL, 12.2 mmol, 1.50 eq) at 0° C. The reaction was stirred at 25° C. for 0.5 h. The mixture was concentrated and the obtained residue was purified by standard methods to afford phenyl (2,6-dimethylpyridin-4-yl)carbamate.

Compound 159: General procedure A with variant iii) was used for the preparation from compound VI-A employing phenyl (3-(difluoromethoxy)-4-methylphenyl)carbamate. ¹H NMR (400 MHz, DMSO-d₆) δ=10.99 (br s, 1H), 8.82 (s, 1H), 7.66 (s, 1H), 7.57 (s, 2H), 7.43 (s, 1H), 7.09 (t, J=74.0 Hz, 1H), 7.14-7.07 (m, 2H), 6.83 (br t, J=6.0 Hz, 1H), 5.12 (dd, J=5.1, 13.2 Hz, 1H), 4.49-4.26 (m, 4H), 3.00-2.84 (m, 1H), 2.64-2.57 (m, 1H), 2.40 (dq, J=4.6, 13.2 Hz, 1H), 2.14 (s, 3H), 2.05-1.95 (m, 1H). MS (ESI) m/z 473.1 [M+H]⁺

### Preparation of phenyl (3-(difluoromethoxy)-4-methylphenyl)carbamate

Step 1: To a solution of 2-methyl-5-nitrophenol (5.00 g, 32.7 mmol, 1.00 eq) and sodium 2-chloro-2,2-difluoroacetate (12.4 g, 81.6 mmol, 2.50 eq) in dimethyl formamide (50.0 mL) was added caesium carbonate (21.3 g, 65.3 mmol, 2.00 eq) in portions. The reaction was stirred at 100° C. for 2 h. The mixture was diluted with water (800 mL) and extracted with ethyl acetate (3×100 mL). The combined organic layers were washed with brine (80.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 2-(difluoromethoxy)-1-methyl-4-nitrobenzene.

Step 2: To a solution of 2-(difluoromethoxy)-1-methyl-4-nitrobenzene (4.85 g, 23.8 mmol, 1.00 eq) and ammonium chloride (6.39 g, 119 mmol, 5.00 eq) in methanol (40.0 mL) and water (40.0 mL) was added iron powder (4.00 g, 71.6 mmol, 3.00 eq) in portions. The reaction was stirred at 80° C. for 2 h. The mixture was filtered, and the filtrate was concentrated under reduced pressure to give a residue. Water (100 mL) was added, and the mixture was extracted with ethyl acetate (3×50.0 mL). The combined organic layers were washed with brine (20 mL) and dried over anhydrous sodium sulfate, filtered, and concentrated to give 3-(difluoromethoxy)-4-methylaniline.

Step 3: To a solution of 3-(difluoromethoxy)-4-methylaniline (1.00 g, 5.78 mmol, 1.00 eq) and pyridine (1.40 mL, 17.3 mmol, 3.00 eq) in acetonitrile (10.0 mL) was added phenyl chloroformate (1.09 mL, 8.66 mmol, 1.50 eq) dropwise. The reaction was stirred at 25° C. for 12 h. The mixture was diluted with water (150 mL) and extracted with ethyl acetate (3×50.0 mL). The combined organic layers were washed with brine (20.0 mL), dried over anhydrous sodium sulfate, filtered, and concentrated and the obtained residue was purified by standard methods to afford phenyl (3-(difluoromethoxy)-4-methylphenyl)carbamate.

Compound 160: General procedure A with variant iii) was used for the preparation from compound VI-A employing phenyl (3-chloro-4-methyl-5-((4-methylmorpholin-3-yl)methyl)phenyl)carbamate. ¹H NMR (400 MHz, DMSO-d₆) δ=10.98 (br s, 1H), 8.74 (s, 1H), 7.66 (s, 1H), 7.57 (s, 3H), 7.02 (d, J=2.0 Hz, 1H), 6.82 (br t, J=5.6 Hz, 1H), 5.12 (dd, J=5.2, 13.2 Hz, 1H), 4.50-4.26 (m, 4H), 3.68-3.59 (m, 1H), 3.57-3.49 (m, 1H), 3.17 (dd, J=7.8, 11.2 Hz, 1H), 3.07 (br dd, J=3.4, 13.2 Hz, 1H), 2.98-2.84 (m, 1H), 2.72-2.57 (m, 3H), 2.43-2.37 (m, 2H), 2.34 (s, 3H), 2.29-2.17 (m, 5H), 2.01 (dt, J=1.8, 6.2 Hz, 1H). MS (ESI) m/z 554.2 [M+H]⁺

### Preparation of phenyl (3-chloro-4-methyl-5-((4-methylmorpholin-3-yl)methyl)phenyl)carbamate

Step 1: A mixture of 2-methyl-5-nitrobenzoic acid (20.0 g, 110 mmol, 1.00 eq) and 1,3-dichloro-5,5-dimethylimidazolidine-2,4-dione (21.8 g, 110 mmol, 1.00 eq) in sulfuric acid (20.0 mL) was stirred at 80° C. for 10 h. The mixture was poured into ice water (about 300 mL) under stirring. The resulting precipitate was collected by filtration and washed with water to afford 3-chloro-2-methyl-5-nitrobenzoic acid.

Step 2: To a solution of 3-chloro-2-methyl-5-nitrobenzoic acid (24.0 g, 111 mmol, 1.00 eq) in tetrahydrofuran (200 mL) was added borane dimethyl sulfide complex (10.0 M, 22.3 mL, 2.00 eq) at 0° C. The reaction was stirred at 25° C. for 10 h. Water (50.0 mL) was added to quench the reaction, and the mixture was extracted with ethyl acetate (3×200 mL). The combined organic layers were washed with brine (100 mL), dried over sodium sulfate, filtered, and concentrated under reduced pressure to afford (3-chloro-2-methyl-5-nitrophenyl)methanol.

Step 3: To a solution of (3-chloro-2-methyl-5-nitrophenyl)methanol (23.0 g, 114 mmol, 1.00 eq) in dichloromethane (200 mL) was added thionyl chloride (41.4 mL, 570 mmol, 5.00 eq). The reaction was stirred at 25° C. for 10 h. The mixture was poured into ice water (50.0 mL) and extracted with dichloromethane (3×150 mL). The combined organic layers were washed with saturated aqueous sodium bicarbonate (2×50.0 mL) and brine (50.0 mL), dried over magnesium sulfate, filtered, and concentrated under reduced pressure to afford 1-chloro-3-(chloromethyl)-2-methyl-5-nitrobenzene.

Step 4: To a solution of sodium hydride 60% purity (1.00 g, 25.0 mmol, 1.10 eq) in dimethylformamide (50.0 mL) was added diethyl 2-acetamidomalonate (5.92 g, 27.3 mmol, 1.20 eq) at 0° C. After 5 mins, 1-chloro-3-(chloromethyl)-2-methyl-5-nitrobenzene (5.00 g, 22.7 mmol, 1.00 eq) was added. The reaction was stirred at 25° C. for 10 h. Water (50.0 mL) was added to quench the reaction, and the mixture was extracted with ethyl acetate (3×100 mL). The combined organic layers were washed with brine (3×50.0 mL), dried over sodium sulfate, filtered, and concentrated under reduced pressure to afford diethyl 2-acetamido-2-(3-chloro-2-methyl-5-nitrobenzyl)malonate.

Step 5: To a solution of 2-amino-3-(3-chloro-2-methyl-5-nitrophenyl)propanoic acid (3.00 g, 11.6 mmol, 1.00 eq) in tetrahydrofuran (30.0 mL) was added borane dimethyl sulfide complex (10.0 M, 3.48 mL, 3.00 eq). The reaction was stirred at 70° C. for 10 h. Methanol (20.0 mL) was added at 0° C. to quench the reaction, and the mixture was concentrated under reduced pressure to afford 2-amino-3-(3-chloro-2-methyl-5-nitrophenyl) propan-1-ol.

Step 6: To a solution of 2-amino-3-(3-chloro-2-methyl-5-nitrophenyl)propan-1-ol (3.00 g, 12.3 mmol, 1.00 eq) and triethylamine (2.05 mL, 14.7 mmol, 1.20 eq) in tetrahydrofuran (30.0 mL) was added 2-chloroacetyl chloride (1.17 mL, 14.7 mmol, 1.20 eq) at 0° C. The reaction was stirred at 25° C. for 1 h. Water (10.0 mL) was added to quench the reaction, and the mixture was extracted with ethyl acetate (3×50.0 mL). The combined organic layers were washed with brine (20.0 mL), dried over sodium sulfate, filtered, and concentrated under reduced pressure to give a residue. The residue was purified by silica gel column chromatography (petroleum ether/ethyl acetate=1/1) to afford 2-chloro-N-(1-(3-chloro-2-methyl-5-nitrophenyl)-3-hydroxypropan-2-yl)acetamide.

Step 7: To a solution of 2-chloro-N-(1-(3-chloro-2-methyl-5-nitrophenyl)-3-hydroxypropan-2-yl)acetamide (1.20 g, 3.74 mmol, 1.00 eq) in tert-butyl alcohol (3.00 mL) was added potassium tert-butoxide (839 mg, 7.47 mmol, 2.00 eq). The reaction was stirred at 100° C. for 0.5 h. The mixture was concentrated under reduced pressure to give a residue. The residue was diluted with water (10.0 mL) and extracted with ethyl acetate (3×30.0 mL). The combined organic layers were washed with brine (20.0 mL), dried over sodium sulfate, filtered, and concentrated under reduced pressure to give a residue. The residue was purified by silica gel column chromatography (dichloromethane/methanol=5/1) to afford 5-(3-chloro-2-methyl-5-nitrobenzyl)morpholin-3-one.

Step 8: To a solution of 5-(3-chloro-2-methyl-5-nitrobenzyl)morpholin-3-one (240 mg, 843 μmol, 1.00 eq) in tetrahydrofuran (3.00 mL) was added borane dimethyl sulfide complex (10.0 M, 253 μL, 3.00 eq). The reaction was stirred at 70° C. for 10 h. Methanol (10.0 mL) was added to quench the reaction, and the mixture was concentrated under reduced pressure to afford 3-(3-chloro-2-methyl-5-nitrobenzyl)morpholine.

Step 9: To a solution of 3-(3-chloro-2-methyl-5-nitrobenzyl)morpholine (0.150 g, 554 μmol, 1.00 eq) and formaldehyde 37% purity (0.90 mL, 12.1 mmol, 21.8 eq) in methanol (2.00 mL) was added acetic acid (63.4 μL, 1.11 mmol, 2.00 eq). After 0.5 h, sodium cyanoborohydride (174 mg, 2.77 mmol, 5.00 eq) was added, and the reaction was stirred at 25° C. for 10 h. Water (15.0 mL) was added to quench the reaction, and the mixture was extracted with dichloromethane (3×30.0 mL). The combined organic layers were washed with brine (15.0 mL), dried over sodium sulfate, filtered, and concentrated under reduced pressure to give a residue. The residue was purified by reversed phase preparative HPLC to afford 3-(3-chloro-2-methyl-5-nitrobenzyl)-4-methylmorpholine.

Step 10: A mixture of 3-(3-chloro-2-methyl-5-nitrobenzyl)-4-methylmorpholine (0.140 g, 492 μmol, 1.00 eq), ferrous powder (137 mg, 2.46 mmol, 5.00 eq) and ammonium chloride (26.3 mg, 492 μmol, 1.00 eq) in ethanol (2.00 mL) and water (1.00 mL) was stirred at 60° C. for 10 h. The mixture was concentrated under reduced pressure to give a residue. Water (15.0 mL) was added and the mixture was extracted with ethyl acetate (3×25.0 mL). The combined organic layers were washed with brine (10.0 mL), dried over sodium sulfate, filtered, and concentrated under reduced pressure to afford 3-chloro-4-methyl-5-((4-methylmorpholin-3-yl)methyl)aniline.

Step 11: To a solution of 3-chloro-4-methyl-5-((4-methylmorpholin-3-yl)methyl)aniline (0.100 g, 393 μmol, 1.00 eq) and potassium carbonate (109 mg, 785 μmol, 2.00 eq) in acetone (2.00 mL) was added phenyl chloroformate (59.0 μL, 471 μmol, 1.20 eq) at 0° C. The reaction was stirred at 25° C. for 10 h. The mixture was diluted with water (10.0 mL) and extracted with ethyl acetate (3×25.0 mL). The combined organic layers were washed with brine (10.0 mL), dried over sodium sulfate, filtered, and concentrated under reduced pressure to afford phenyl (3-chloro-4-methyl-5-((4-methylmorpholin-3-yl) methyl)phenyl)carbamate 1.

Compound 201: General procedure B with variant i) was used for the preparation from compound VIII-B employing phenyl (5-chloro-2-methoxy-4-methylphenyl)carbamate. ¹H NMR (400 MHz, DMSO-d₆) δ=11.17-10.81 (m, 1H), 8.87 (s, 1H), 7.82 (s, 1H), 7.75-7.57 (m, 3H), 7.03 (s, 1H), 5.26 (s, 2H), 5.14 (br dd, J=5.1, 13.2 Hz, 1H), 4.53-4.40 (m, 1H), 4.39-4.29 (m, 1H), 3.80 (s, 3H), 2.92 (br s, 1H), 2.65-2.57 (m, 1H), 2.44-2.38 (m, 1H), 2.29 (s, 3H), 2.07-1.97 (m, 1H). MS (ESI) m/z 472.1 [M+H]⁺

Step 1: To a solution of 4-chloro-5-methyl-2-nitrophenol (500 mg, 2.67 mmol, 1.00 eq) in dimethylformamide (3.00 mL) were added potassium carbonate (740 mg, 5.35 mmol, 2.01 eq) and methyl iodide (0.17 mL, 2.73 mmol, 1.02 eq). The reaction was stirred at 25° C. for 2 h. The mixture was extracted with water/ethyl acetate (2.00 mL/2.00 mL). The organic layer was collected and concentrated to afford 1-chloro-4-methoxy-2-methyl-5-nitrobenzene (490 mg, crude) as a yellow solid.

Step 2: To a solution of 1-chloro-4-methoxy-2-methyl-5-nitrobenzene (490 mg, 2.43 mmol, 1.00 eq) in methanol (9.00 mL) and water (3.00 mL) were added iron power (680 mg, 12.1 mmol, 5.01 eq) and ammonium chloride (1.04 g, 19.4 mmol, 8.00 eq). The reaction was stirred at 80° C. for 2 h. The mixture was filtered, and the filtrate was concentrated under reduced pressure to give a concentrated aqueous solution. The solution was extracted with water/ethyl acetate. The organic layers were collected and concentrated under reduced pressure to afford 5-chloro-2-methoxy-4-methylaniline.

Step 3: To a solution of 5-chloro-2-methoxy-4-methylaniline (200 mg, 1.17 mmol, 1.00 eq) in acetonitrile (3.00 mL) was added pyridine (0.47 mL, 5.82 mmol, 4.99 eq), phenyl chloroformate (0.22 mL, 1.75 mmol, 1.50 eq) at 0° C. The reaction was stirred at 0° C. for 2 h. The mixture was concentrated and the obtained residue was purified by standard methods to afford phenyl (5-chloro-2-methoxy-4-methylphenyl)carbamate (690 mg, crude) as an off-white solid.

Compound 202: General procedure B with variant i) was used for the preparation from compound VIII-B employing phenyl (3-chloro-4-methyl-5-(morpholinomethyl) phenyl)carbamate. ¹H NMR (400 MHz, DMSO-d₆) δ=11.00 (s, 1H), 9.90 (br s, 1H), 8.14 (s, 1H), 7.80 (s, 1H), 7.70-7.62 (m, 2H), 7.55 (s, 1H), 7.43-7.28 (m, 1H), 5.28 (s, 2H), 5.13 (dd, J=5.1, 13.3 Hz, 1H), 4.51-4.44 (m, 1H), 4.39-4.31 (m, 1H), 3.57 (br s, 4H), 3.41 (br s, 2H), 2.96-2.87 (m, 1H), 2.63 (br s, 1H), 2.46-2.32 (m, 5H), 2.29 (s, 3H), 2.06-1.98 (m, 1H). MS (ESI) m/z 541.2 [M+H]⁺

Step 1: To a solution of 2-methyl-5-nitro-benzoic acid (10.0 g, 55.2 mmol, 1.00 eq) in concentrated sulfuric acid (30.0 mL, 98% purity) was added 1,3-dichloro-5,5-dimethyl-imidazolidine-2,4-dione (13.0 g, 66.0 mmol, 1.20 eq). The mixture was stirred at 80° C. for 12 h. After cooling to room temperature, the mixture was poured into ice-water. The resulting white precipitate was collected by filtration and dried under reduced pressure to afford 3-chloro-2-methyl-5-nitro-benzoic acid.

Step 2: To a solution of 3-chloro-2-methyl-5-nitro-benzoic acid (15.0 g, 69.6 mmol, 1.00 eq) in tetrahydrofuran (100 mL) was added borane dimethyl sulfide complex (10 M, 14.0 mL, 140.0 mmol, 2.01 eq). The mixture was stirred at 20° C. for 12 h. The mixture was quenched with methanol (10.0 mL) at 0° C. and then concentrated under reduced pressure to give a residue. The residue was diluted with ethyl acetate (200 mL) and adjusted pH=8 with aqueous sodium bicarbonate (10%, 200 mL). The organic layer was separated and concentrated under reduced pressure to afford (3-chloro-2-methyl-5-nitrophenyl)methanol.

Step 3: To a solution of (3-chloro-2-methyl-5-nitro-phenyl)methanol (11.0 g, 54.5 mmol, 1 eq) (crude) in dichloromethane (200 mL) was added carbon tetrabromide (21 g, 63.32 mmol, 1.16 eq) and triphenylphosphine (18.6 g, 70.9 mmol, 1.30 eq) at 0° C. The mixture was stirred at 20° C. for 1 h. The mixture was concentrated under reduced pressure to give a residue. The residue was triturated with petroleum ether/ethyl acetate=1/1 (200 mL) and filtered. The filtrate was concentrated under reduced pressure to give a residue. The residue was purified by silica gel column chromatography (petroleum ether/ethyl acetate=8/1) to afford 1-(bromomethyl)-3-chloro-2-methyl-5-nitrobenzene.

Step 4: To a solution of 1-(bromomethyl)-3-chloro-2-methyl-5-nitro-benzene (16.0 g, 60.5 mmol, 1.00 eq) in acetonitrile (150 mL) was added morpholine (7.98 mL, 90.7 mmol, 1.50 eq), potassium carbonate (25.0 g, 181 mmol, 2.99 eq) and potassium iodide (1.00 g, 6.05 mmol, 0.100 eq). The reaction was stirred at 80° C. for 12 h. After cooling to room temperature, the mixture was filtered, and the filtrate was concentrated under reduced pressure to give a residue. The residue was purified by silica gel column chromatography (petroleum ether/ethyl acetate=8/1) to give 4-(3-chloro-2-methyl-5-nitrobenzyl)morpholine.

Step 5: To a solution of 4-(3-chloro-2-methyl-5-nitrobenzyl)morpholine (9.70 g, 35.8 mmol, 1.00 eq) in methanol (100 mL) and water (30.0 mL) was added ammonium chloride (15.0 g, 280 mmol, 7.83 eq) and ferrous powder (10.0 g, 179 mmol, 5.00 eq). The reaction was stirred at 80° C. for 2 h. The mixture was filtered, and methanol was removed under reduced pressure. The remaining aqueous solution was extracted with ethyl acetate (2×100 mL). The combined organic layers were washed with brine (20.0 mL), dried over sodium sulfate, filtered, and concentrated under reduced pressure to afford 3-chloro-4-methyl-5-(morpholinomethyl)aniline.

Step 6: To a solution of 3-chloro-4-methyl-5-(morpholinomethyl)aniline (1.00 g, 4.15 mmol, 1.00 eq) and pyridine (1.01 mL, 12.4 mmol, 3.00 eq) in acetonitrile (20.0 mL) was added phenyl chloroformate (0.52 mL, 4.15 mmol, 1.00 eq) at 0° C. The reaction was stirred at 20° C. for 2 h. The mixture was diluted with water (20.0 mL). The resulting precipitate was collected by filtration and dried to afford phenyl (3-chloro-4-methyl-5-(morpholinomethyl)phenyl)carbamate.

Compound 203: General procedure B with variant i) was used for the preparation from compound VIII-B employing phenyl (3-(trifluoromethoxy)phenyl)carbamate. ¹H NMR (400 MHz, DMSO-d₆) δ=11.00 (s, 1H), 10.15 (s, 1H), 7.81 (s, 1H), 7.72-7.68 (m, 1H), 7.67-7.62 (m, 1H), 7.59 (s, 1H), 7.45-7.39 (m, 2H), 7.03-6.96 (m, 1H), 5.30 (s, 2H), 5.13 (dd, J=5.2, 13.3 Hz, 1H), 4.52-4.44 (m, 1H), 4.39-4.31 (m, 1H), 2.97-2.87 (m, 1H), 2.63 (br d, J=2.4 Hz, 1H), 2.47-2.35 (m, 1H), 2.02 (dtd, J=2.0, 5.1, 12.5 Hz, 1H). MS (ESI) m/z 478.1 [M+H]⁺

Step 1: To a solution of 1-nitro-3-(trifluoromethoxy)benzene (1.00 g, 4.83 mmol, 1.00 eq) in methanol (15.0 mL) and water (5.00 mL) was added ammonium chloride (2.07 g, 38.6 mmol, 8.00 eq) and iron power (1.35 g, 24.1 mmol, 5.00 eq). The reaction was stirred at 80° C. for 2 h. The mixture was filtered, and the solvents were mostly removed under reduced pressure to give a concentrated solution. The solution was triturated with ethyl acetate/water (20.0 ml/10.0 ml), and a drop of saturated sodium bicarbonate solution was added. The organic layer was collected and concentrated under reduced pressure to afford 3-(trifluoromethoxy)aniline.

Step 2: To a solution of 3-(trifluoromethoxy)aniline (0.15 mL, 1.10 mmol, 1.00 eq) in acetonitrile (5.00 mL) was added pyridine (0.44 mL, 5.50 mmol, 5.00 eq). The reaction was stirred for 0.5 h, then phenyl chloroformate (0.16 mL, 1.32 mmol, 1.20 eq) was added at 0° C. The reaction was stirred for 1.5 h at 0° C. The mixture was concentrated under reduced pressure to give a residue. The residue was diluted with water (10.0 ml) and ethyl acetate (30.0 mL). The organic layer was collected and concentrated and the obtained residue was purified by standard methods to afford phenyl (3-(trifluoromethoxy)phenyl)carbamate.

Compound 204: General procedure B with variant i) was used for the preparation from compound VIII-B employing phenyl (3-(morpholinomethyl)-5-(trifluoromethoxy)phenyl)carbamate. ¹H NMR (400 MHz, DMSO-d₆) δ=10.99 (s, 1H), 10.11 (s, 1H), 7.80 (s, 1H), 7.71-7.60 (m, 2H), 7.52-7.37 (m, 2H), 6.91 (s, 1H), 5.29 (s, 2H), 5.18-5.07 (m, 1H), 4.53-4.29 (m, 2H), 3.62-3.52 (m, 4H), 3.45 (s, 2H), 2.98-2.85 (m, 1H), 2.60 (br d, J=18.1 Hz, 1H), 2.43-2.38 (m, 1H), 2.34 (br s, 4H), 2.05-1.96 (m, 1H). MS (ESI) m/z 577.1 [M+H]⁺

Step 1: To a solution of 3-hydroxy-5-nitro-benzoic acid (4.50 g, 24.6 mmol, 1.00 eq) and morpholine (2.57 g, 29.5 mmol, 2.60 mL, 1.2 eq) in dichloromethane (100 mL) was added O-(7-azabenzotriazol-1-yl)-N,N,N,N-tetramethyluronium hexafluorophosphate (11.2 g, 29.5 mmol, 1.20 eq) and triethylamine (5.06 mL, 49.2 mmol, 2.00 eq). The reaction was stirred at 20° C. for 12 h. The mixture was diluted with water (150 mL) and extracted with ethyl acetate (3×100 mL). The combined organic layers were washed with brine (100 mL), dried over sodium sulfate, filtered, and concentrated under reduced pressure to give a residue. The residue was purified by silica gel column chromatography (petroleum ether/ethyl acetate=0/1) to afford (3-hydroxy-5-nitrophenyl)(morpholino) methanone.

Step 2: To a solution of (3-hydroxy-5-nitro-phenyl)-morpholino-methanone (2.40 g, 9.52 mmol, 1.00 eq) in toluene (200 mL) were added trimethyl(trifluoromethyl)silane (6.77 g, 47.6 mmol, 5.00 eq), silver trifluoromethanesulfonate (12.2 g, 47.6 mmol, 5.00 eq), 2-fluoropyridine (4.09 mL, 47.6 mmol, 5.00 eq), caesium fluoride (2.10 mL, 57.1 mmol, 6.00 eq), N-fluorobenzenesulfonimide (6.00 g, 19.0 mmol, 2.00 eq) and 1-(chloromethyl)-4-fluoro-1,4-diazoniabicyclo(2.2.2)octane bis(tetrafluoroborate) (6.74 g, 19.0 mmol, 2.00 eq). The reaction was stirred at 25° C. under a nitrogen atmosphere for 12 h. The mixture was filtered, and the filtrate was concentrated under reduced pressure to give a residue. The residue was diluted with water (100 mL) and extracted with ethyl acetate (3×100 mL). The combined organic layers were washed with brine (100 mL), dried over anhydrous sodium sulfate, filtered, and the filtrate was concentrated to give a residue. The residue was purified by silica gel column chromatography (petroleum ether/ethyl acetate=1/1) to afford morpholino(3-nitro-5-(trifluoromethoxy) phenyl)methanone.

Step 3: To a solution of morpholino(3-nitro-5-(trifluoromethoxy)phenyl)methanone (2.60 g, 8.12 mmol, 1.00 eq) in tetrahydrofuran (30.0 mL) was added borane dimethyl sulfide complex (10.0 M, 1.62 mL, 2.00 eq) dropwise at 0° C. under nitrogen atmosphere. The reaction was stirred at 60° C. for 4 h. The mixture was quenched with methanol (9.00 mL) and concentrated to give a residue. The residue was purified by reversed phase preparative HPLC to afford 4-(3-nitro-5-(trifluoromethoxy)benzyl)morpholine.

Step 4: To a solution of 4-(3-nitro-5-(trifluoromethoxy)benzyl)morpholine (1.10 g, 3.59 mmol, 1.00 eq) in methanol (50.0 mL) and water (50.0 mL) were added iron powder (1.40 g, 25.14 mmol, 7.00 eq) and ammonium chloride (1.34 g, 25.14 mmol, 7.00 eq). The reaction was stirred at 80° C. for 3 h. The mixture was filtered over Celite, and the filtrate was concentrated under reduced pressure to afford 3-(morpholinomethyl)-5-(trifluoromethoxy)aniline.

Step 5: To a solution of 3-(morpholinomethyl)-5-(trifluoromethoxy)aniline (580 mg, 2.10 mmol, 1.00 eq) in acetonitrile (3.00 mL) was added phenyl chloroformate (0.39 mL, 3.15 mmol, 1.50 eq) and pyridine (0.5 mL, 6.30 mmol, 3.00 eq) at 0° C. The reaction was stirred at 25° C. for 12 h. The mixture was diluted with water (100 mL) and extracted with ethyl acetate (3×30.0 mL). The combined organic layers were washed with brine (30 mL), dried over anhydrous sodium sulfate, filtered, and the filtrate was concentrated and the obtained residue was purified by standard methods to afford phenyl (3-(morpholinomethyl)-5-(trifluoromethoxy)phenyl)carbamate.

Compound 205: General procedure B with variant i) was used for the preparation from compound VIII-B employing phenyl (3-(difluoromethoxy)phenyl)carbamate. ¹H NMR (400 MHz, DMSO-d₆) δ=11.00 (br s, 1H), 10.05 (s, 1H), 7.80 (s, 1H), 7.71-7.67 (m, 1H), 7.67 -7.62 (m, 1H), 7.40 (br s, 1H), 7.37 (s, 1H), 7.36-7.28 (m, 2H), 7.19 (t, J=76 Hz, 1H), 6.81 (br d, J=7.5 Hz, 1H), 5.29 (s, 2H), 5.13 (dd, J=5.1, 13.3 Hz, 1H), 4.52-4.44 (m, 1H), 4.39-4.30 (m, 1H), 2.97-2.87 (m, 1H), 2.64-2.58 (m, 1H), 2.48-2.37 (m, 1H), 2.07-1.98 (m, 1H). MS (ESI) m/z 460.1 [M+H]⁺

Step 1: To a solution of 1-(difluoromethoxy)-3-nitrobenzene (2.00 g, 10.6 mmol, 1.00 eq) in methanol (15.0 mL) and water (5.00 mL) was added iron powder (2.95 g, 52.9 mmol, 5.00 eq) and ammonium chloride (4.53 g, 84.6 mmol, 8.00 eq). The reaction was stirred at 80° C. for 2 h. The reaction solution was filtered, and the filtrate was extracted with ethyl acetate. The combined organic layers were washed with brine, dried with anhydrous sodium sulfate, filtered, and concentrated under reduced pressure to afford 3-(difluoromethoxy)aniline.

Step 2: To a solution of 3-(difluoromethoxy)aniline (1.00 g, 6.28 mmol, 1.00 eq) and pyridine (2.54 mL, 31.4 mmol, 5.00 eq) in acetonitrile (5.00 mL) was added phenyl chloroformate (0.94 mL, 7.54 mmol, 1.20 eq). The mixture was stirred at 25° C. for 3 h. The mixture was concentrated and the obtained residue was purified by standard methods to afford phenyl (3-(difluoromethoxy)phenyl)carbamate.

Compound 206: General procedure B with variant i) was used for the preparation from compound VIII-B employing phenyl(5-chloro-6-methylpyridin-3-yl)carbamate. ¹H NMR (400 MHz, DMSO-d₆) δ=11.00 (s, 1H), 10.18 (br s, 1H), 8.50-8.42 (m, 1H), 7.99 (s, 1H), 7.80 (s, 1H), 7.72-7.66 (m, 1H), 7.66-7.62 (m, 1H), 5.30 (s, 2H), 5.17-5.08 (m, 1H), 4.52-4.43 (m, 1H), 4.38-4.30 (m, 1H), 2.98-2.85 (m, 1H), 2.65-2.56 (m, 1H), 2.46 (s, 3H), 2.44-2.34 (m, 1H), 2.05-1.95 (m, 1H). MS (ESI) m/z 443.2 [M+H]⁺

Step 1: To a solution of 3-chloro-2-methyl-5-nitropyridine (500 mg, 2.90 mmol, 1.00 eq) in methanol (5.00 mL) and water (5.00 mL) were added iron powder (1.13 g, 20.3 mmol, 7.00 eq) and ammonium chloride (1.08 g, 20.3 mmol, 7.00 eq). The reaction was stirred at 80° C. for 2 h. The mixture was filtered over Celite, and the filtrate was concentrated under reduced pressure to afford 5-chloro-6-methylpyridin-3-amine.

Step 2: To a solution of 5-chloro-6-methylpyridin-3-amine (200 mg, 1.40 mmol, 1.00 eq) in acetonitrile (2.00 mL) were added phenyl chloroformate (0.26 mL, 2.10 mmol, 1.50 eq) and pyridine (0.34 mL, 4.21 mmol, 3.00 eq). The reaction was stirred at 25° C. for 12 h. The mixture was diluted with ethyl acetate (30.0 mL) and water (50.0 mL). The organic layer was separated, and the aqueous phase was extracted with ethyl acetate (3×100 mL). The combined organic layers were dried over sodium sulfate, filtered, and concentrated and the obtained residue was purified by standard methods to afford phenyl (5-chloro-6-methylpyridin-3-yl)carbamate.

Compound 207: General procedure B with variant i) was used for the preparation from compound VIII-B employing phenyl (2,6-dimethylpyridin-4-yl)carbamate. ¹H NMR (400 MHz, DMSO-d₆) δ=10.99 (s, 1H), 10.10 (s, 1H), 8.18 (s, 1H), 7.80 (s, 1H), 7.71-7.60 (m, 2H), 7.12 (s, 2H), 5.29 (s, 2H), 5.12 (dd, J=5.1, 13.3 Hz, 1H), 4.51-4.43 (m, 1H), 4.39-4.30 (m, 1H), 2.91 (ddd, J=5.4, 13.7, 17.4 Hz, 1H), 2.64-2.57 (m, 1H), 2.40 (br dd, J=4.6, 13.1 Hz, 1H), 2.33 (s, 6H), 2.07-1.97 (m, 1H). MS (ESI) m/z 423.1 [M+H]⁺

To a solution of 2,6-dimethylpyridin-4-amine (1.00 g, 8.19 mmol, 1.00 eq) in acetonitrile (20.0 mL) was added pyridine (3.30 mL, 40.9 mmol, 5.00 eq) and phenyl chloroformate (1.54 mL, 12.2 mmol, 1.50 eq) at 0° C. The reaction was stirred at 25° C. for 0.5 h. The mixture was concentrated and the obtained residue was purified by standard methods to afford phenyl (2,6-dimethylpyridin-4-yl)carbamate.

Compound 208: General procedure B with variant i) was used for the preparation from compound VIII-B employing phenyl (3,5-dimethylphenyl)carbamate. ¹H NMR (400 MHz, DMSO-d₆) δ=10.9 (br s, 1H), 9.65 (s, 1H), 7.78 (s, 1H), 7.70-7.59 (m, 2H), 7.09 (s, 2H), 6.64 (s, 1H), 5.25 (s, 2H), 5.13 (dd, J=5.1, 13.4 Hz, 1H), 4.52-4.42 (m, 1H), 4.39-4.28 (m, 1H), 2.99-2.84 (m, 1H), 2.60 (td, J=2.1, 15.3 Hz, 1H), 2.40 (br dd, J=4.4, 13.1 Hz, 1H), 2.21 (s, 6H), 2.06-1.98 (m, 1H). MS (ESI) m/z 422.1 [M+H]⁺

To a solution of 3,5-dimethylaniline (0.51 mL, 4.13 mmol, 1.00 eq) in acetonitrile (10.00 mL) was added pyridine (1.67 mL, 20.6 mmol, 5.00 eq) and phenyl chloroformate (1.03 mL, 8.25 mmol, 2.00 eq) at 0° C. in portions. The reaction was stirred at 25° C. for 1 h. The mixture was concentrated under reduced pressure to give a residue, which was purified by silica gel column chromatography (petroleum ether/ethyl acetate=1/0 to 3/1) to afford phenyl (3,5-dimethylphenyl) carbamate.

Compound 209: General procedure B with variant i) was used for the preparation from compound VIII-B employing phenyl (3-chloro-4-fluorophenyl)carbamate. ¹H NMR (400 MHz, DMSO-d₆) δ=10.99 (br s, 1H), 10.05 (br s, 1H), 7.79 (s, 1H), 7.74-7.61 (m, 3H), 7.44-7.31 (m, 2H), 5.28 (s, 2H), 5.12 (dd, J=5.1, 13.3 Hz, 1H), 4.52-4.41 (m, 1H), 4.39-4.29 (m, 1H), 2.98-2.85 (m, 1H), 2.60 (br dd, J=2.1, 15.4 Hz, 1H), 2.42 (dt, J=4.4, 13.3 Hz, 1H), 2.05-1.96 (m, 1H). MS (ESI) m/z 446.1 [M+H]⁺

To a solution of 3-chloro-4-fluoroaniline (1.00 g, 6.87 mmol, 1.00 eq) in acetonitrile (10.0 mL) was added pyridine (2.77 mL, 34.4 mmol, 5.00 eq) and phenyl chloroformate (1.72 mL, 13.7 mmol, 2.00 eq) in portions at 0° C. The reaction was stirred at 25° C. for 0.5 h. The mixture was concentrated and the obtained residue was purified by standard methods to afford phenyl (3-chloro-4-fluorophenyl)carbamate.

Compound 210: General procedure B with variant i) was used for the preparation from compound VIII-B employing phenyl (3-chloro-4-methylphenyl)carbamate. ¹H NMR (400 MHz, DMSO-d₆) δ=11.00 (s, 1H), 9.93 (s, 1H), 7.80 (s, 1H), 7.74-7.54 (m, 3H), 7.37-7.19 (m, 2H), 5.28 (s, 2H), 5.13 (dd, J=5.1, 13.2 Hz, 1H), 4.53-4.44 (m, 1H), 4.38-4.31 (m, 1H), 2.98-2.87 (m, 1H), 2.61 (br d, J=17.6 Hz, 1H), 2.41 (br dd, J=4.4, 13.2 Hz, 1H), 2.26 (s, 3H), 2.06-1.97 (m, 1H). MS (ESI) m/z 442.1 [M+H]⁺

Preparation of phenyl (3-chloro-4-methylphenyl)carbamate: To a solution of 3-chloro-4-methylaniline (5.00 g, 35.3 mmol, 1.00 eq) in acetonitrile (50.0 mL) was added pyridine (5.70 mL, 70.6 mmol, 2.00 eq) and phenyl chloroformate (4.87 mL, 38.8 mmol, 1.10 eq) at 0° C. The mixture was stirred at 30° C. for 2 h. The mixture was concentrated and the obtained residue was purified by standard methods to afford phenyl (3-chloro-4-methylphenyl)carbamate.

Compound 211: General procedure B with variant i) was used for the preparation from compound VIII-B employing phenyl (3-chloro-5-(trifluoromethoxy)phenyl)carbamate. ¹H NMR (400 MHz, DMSO-d₆) δ=11.00 (s, 1H), 10.35 (s, 1H), 7.81 (s, 1H), 7.73-7.68 (m, 1H), 7.67-7.62 (m, 1H), 7.60-7.49 (m, 2H), 7.17 (s, 1H), 5.31 (s, 2H), 5.13 (dd, J=5.1, 13.3 Hz, 1H), 4.53-4.44 (m, 1H), 4.39-4.31 (m, 1H), 2.99-2.85 (m, 1H), 2.64-2.58 (m, 1H), 2.41 (dd, J=4.4, 13.0 Hz, 1H), 2.07-1.95 (m, 1H). MS (ESI) m/z 332.0 [M+H]⁺

To a solution of 3-chloro-5-(trifluoromethoxy)aniline (150 mg, 708 μmol, 1.00 eq) in acetonitrile (1.00 mL) were added pyridine (0.29 mL, 3.54 mmol, 5.00 eq) and phenyl chloroformate (0.11 mL, 850 μmol, 1.20 eq) at 0° C. The reaction was stirred at 0° C. for 2 h. The mixture was filtered to give a solution, which was purified by standard methods to afford phenyl (3-chloro-5-(trifluoromethoxy)phenyl)carbamate.

Compound 212: General procedure B with variant i) was used for the preparation from compound VIII-B employing phenyl (3-chloro-5-fluorophenyl)carbamate. ¹H NMR (400 MHz, DMSO-d₆) δ=10.99 (s, 1H), 10.25 (s, 1H), 7.80 (s, 1H), 7.74-7.56 (m, 2H), 7.44-7.28 (m, 2H), 7.10-6.96 (m, 1H), 5.30 (s, 2H), 5.18-5.06 (m, 1H), 4.54-4.42 (m, 1H), 4.40-4.31 (m, 1H), 2.98-2.86 (m, 1H), 2.62 (br d, J=2.6 Hz, 1H), 2.46-2.37 (m, 1H), 2.05-1.96 (m, 1H). MS (ESI) m/z 446.0 [M+H]⁺

To a solution of 3-chloro-5-fluoroaniline (1.00 g, 6.87 mmol, 1.00 eq) in acetonitrile (20.0 mL) were added pyridine (2.77 mL, 34.3 mmol, 5.00 eq) and phenyl chloroformate (1.29 mL, 10.3 mmol, 1.50 eq) at 0° C. The reaction was stirred at 25° C. for 1 h. The mixture was concentrated and the obtained residue was purified by standard methods to afford phenyl (3-chloro-5-fluorophenyl)carbamate.

Compound 213: General procedure B with variant i) was used for the preparation from compound VIII-B employing phenyl (3-(2-oxa-5-azabicyclo[2.2.1]heptan-5-ylmethyl)-5-chloro-4-methylphenyl)carbamate.

¹H NMR (400 MHz, DMSO-d₆) δ=11.00 (s, 1H), 9.89 (br s, 1H), 8.14 (s, 1H), 7.80 (s, 1H), 7.71-7.62 (m, 2H), 7.54 (br s, 1H), 7.41 (d, J=1.5 Hz, 1H), 5.27 (s, 2H), 5.13 (dd, J=5.0, 13.3 Hz, 1H), 4.46 (s, 1H), 4.37 (s, 2H), 3.91 (s, 1H), 3.72-3.64 (m, 2H), 3.54 (dd, J=1.6, 7.4 Hz, 1H), 3.46 (br s, 1H), 2.97-2.87 (m, 1H), 2.75 (br d, J=9.0 Hz, 1H), 2.61 (br d, J=17.0 Hz, 1H), 2.47 (br d, J=10.3 Hz, 1H), 2.41 (br dd, J=4.6, 13.0 Hz, 1H), 2.26 (s, 3H), 2.07-1.97 (m, 1H), 1.80 (br s, 1H), 1.61 (br d, J=9.3 Hz, 1H). MS (ESI) m/z 553.2 [M+H]⁺

To a solution of 3-(2-oxa-5-azabicyclo[2.2.1]heptan-5-ylmethyl)-5-chloro-4-methylaniline (350 mg, 1.38 mmol, 1.00 eq) in acetonitrile (2.00 mL) were added pyridine (0.56 mL, 6.92 mmol, 5.00 eq) and phenyl chloroformate (0.21 mL, 1.66 mmol, 1.20 eq) at 0° C. The reaction was stirred at 0° C. for 2 h. The mixture was concentrated and the obtained residue was purified by standard methods to afford phenyl (3-(2-oxa-5-azabicyclo[2.2.1]heptan-5-ylmethyl)-5-chloro-4-methylphenyl)carbamate.

Compound 214: General procedure B with variant i) was used for the preparation from compound VIII-B employing phenyl(5-chloro-2-methoxyphenyl)carbamate.

¹H NMR (400 MHz, DMSO-d₆) δ=10.99 (br s, 1H), 8.95 (s, 1H), 7.82 (s, 1H), 7.77 (br d, J=1.5 Hz, 1H), 7.72-7.66 (m, 1H), 7.65-7.60 (m, 1H), 7.13-7.08 (m, 1H), 7.06-7.01 (m, 1H), 5.27 (s, 2H), 5.13 (dd, J=5.1, 13.3 Hz, 1H), 4.51-4.43 (m, 1H), 4.37-4.30 (m, 1H), 3.80 (s, 3H), 2.97-2.86 (m, 1H), 2.60 (td, J=2.1, 15.3 Hz, 1H), 2.40 (br dd, J=4.5, 13.1 Hz, 1H), 2.05-1.97 (m, 1H). MS (ESI) m/z 458.1 [M+H]⁺

To a solution of 5-chloro-2-methoxy aniline (1.00 g, 6.35 mmol, 1.00 eq) in acetonitrile (10.0 mL) were added pyridine (2.56 mL, 31.7 mmol, 5.00 eq) and phenyl chloroformate (1.59 mL, 12.7 mmol, 2.00 eq) in portions at 0° C. The reaction was stirred at 25° C. for 1 h. The mixture was concentrated under reduced pressure to give a residue, which was diluted with water (50.0 mL) and exacted with ethyl acetate (3×30.0 mL). The combined organic layers were washed with water, dried over anhydrous sodium sulfate, filtered, and concentrated under reduced pressure to give a residue. The residue was purified by reversed phase preparative HPLC. The desired fraction was collected, and acetonitrile was removed under reduced pressure. The residual aqueous solution was exacted with ethyl acetate (3×50.0 mL). The combined organic layers were washed with brine, dried over anhydrous sodium sulfate, filtered, and concentrated to afford phenyl(5-chloro-2-methoxyphenyl)carbamate.

Compound 215: General procedure B with variant i) was used for the preparation from compound VIII-B employing phenyl(6-(piperidin-1-yl)pyridin-3-yl)carbamate.

¹H NMR (400 MHz, DMSO-d₆) δ=10.99 (s, 1H), 10.12 (br s, 1H), 8.19 (br s, 1H), 7.95-7.89 (m, 1H), 7.79 (s, 1H), 7.70-7.62 (m, 2H), 7.41 (br d, J=9.3 Hz, 1H), 5.29 (s, 2H), 5.12 (dd, J=5.1, 13.3 Hz, 1H), 4.52-4.44 (m, 1H), 4.39-4.31 (m, 1H), 3.64 (br s, 4H), 2.91 (ddd, J=5.4, 13.6, 17.5 Hz, 1H), 2.63-2.58 (m, 1H), 2.45-2.35 (m, 1H), 2.06-1.96 (m, 1H), 1.63 (br s, 6H). MS (ESI) m/z 478.1[M+H]⁺

Step 1: To a solution of 2-fluoro-5-nitro-pyridine (5.00 g, 35.2 mmol, 1.00 eq) in acetonitrile (50.0 mL) were added potassium carbonate (9.73 g, 70.4 mmol, 2.00 eq) and piperidine (4.17 mL, 42.2 mmol, 1.20 eq). The reaction was stirred at 25° C. for 1 h. The mixture was filtered and concentrated under reduced pressure to afford 5-nitro-2-(1-piperidyl)pyridine.

Step 2: To a solution of 5-nitro-2-(piperidin-1-yl)pyridine (5.20 g, 25.1 mmol, 1.00 eq) and ammonium chloride (6.71 g, 125 mmol, 5.00 eq) in methanol (40.0 mL) and water (10.0 mL) was added iron powder (7.01 g, 126 mmol, 5.00 eq) in portions. The reaction was stirred at 80° C. for 2 h. The mixture was filtered, and the filtrate was concentrated under reduced pressure to give a residue. The residue was diluted with water (150 mL) and extracted with ethyl acetate (3×60.0 mL). The combined organic layers were washed with brine (60.0 mL), dried over sodium sulfate, filtered, and concentrated under reduced pressure to give a residue. The residue was purified by reversed phase preparative HPLC. The desired fraction was collected, diluted with saturated sodium bicarbonate (150 mL), and extracted with ethyl acetate (3×60.0 mL). The combined organic layers were washed with brine (60.0 mL), dried over sodium sulfate, filtered, and concentrated under reduced pressure to afford 6-(piperidin-1-yl)pyridin-3-amine.

Step 3: To a solution of 6-(piperidin-1-yl)pyridin-3-amine (300 mg, 1.69 mmol, 1.00 eq) and pyridine (0.41 mL, 5.08 mmol, 3.00 eq) in acetonitrile (3.00 mL) was added phenyl chloroformate (0.32 mL, 2.54 mmol, 1.50 eq) dropwise at 0° C. The reaction was stirred at 25° C. for 12 h. The mixture was diluted with water (50.0 mL) and extracted with ethyl acetate (3×40.0 mL). The combined organic layers were washed with brine (20.0 mL), dried over sodium sulfate, filtered, and concentrated under reduced pressure to give a residue. The residue was purified by reversed phase preparative HPLC. The desired fraction was collected, diluted with saturated sodium bicarbonate (30 mL), and extracted with ethyl acetate (3×40.0 mL). The combined organic layers were washed with brine (20.0 mL), dried over sodium sulfate, filtered, and concentrated to afford phenyl (6-(piperidin-1-yl)pyridin-3-yl)carbamate.

### Compound 216:

Step 1: To a solution of tert-butyl 3-(hydroxymethyl)pyrrolidine-1-carboxylate (3.00 g, 14.9 mmol, 1.00 eq) and triethylamine (5.19 mL, 37.3 mmol, 2.50 eq) in dichloromethane (30.0 mL) at 0° C. was added methylsulfamoyl chloride (1.50 mL, 19.4 mmol, 1.30 eq) dropwise over 2 min under nitrogen atmosphere. The reaction was then stirred at 25° C. for 2 h. The mixture was diluted with ethyl acetate (100 mL) and water (150 mL). The organic layer was separated, and the aqueous phase was extracted with ethyl acetate (3×100 mL). The combined organic layers were dried over sodium sulfate, filtered, and concentrated under reduced pressure to afford tert-butyl 3-(((methylsulfonyl)oxy)methyl)pyrrolidine-1-carboxylate.

Step 2: A solution of tert-butyl 3-(((methylsulfonyl)oxy)methyl) pyrrolidine-1-carboxylate (3.00 g, 10.8 mmol, 1.00 eq), 3-chloro-5-nitrophenol (2.05 g, 11.8 mmol, 1.10 eq) and caesium carbonate (10.5 g, 32.2 mmol, 3.00 eq) in dimethylformamide (30.0 mL) was stirred at 80° C. for 12 h. The mixture was diluted with ethyl acetate (100 mL) and water (100 mL).

The organic layer was separated, and the aqueous phase was extracted with ethyl acetate (3×80.0 mL). The combined organic layers were dried over sodium sulfate, filtered, and concentrated under reduced pressure to give a residue. The residue was purified by silica gel column chromatography (petroleum ether/ethyl acetate=3/1) to afford tert-butyl 3-((3-chloro-5-nitrophenoxy)methyl)pyrrolidine-1-carboxylate.

Step 3: A solution of tert-butyl 3-((3-chloro-5-nitrophenoxy)methyl)pyrrolidine-1-carboxylate (2.00 g, 5.61 mmol, 1.00 eq), iron powder (2.19 g, 39.2 mmol, 7.00 eq) and ammonium chloride (2.10 g, 39.2 mmol, 7.00 eq) in methanol (30.0 mL) and water (30.0 mL) was stirred at 80° C. for 2 h. The mixture was filtered over Celite, and the filtrate was concentrated under reduced pressure to afford tert-butyl 3-((3-amino-5-chlorophenoxy) methyl)pyrrolidine-1-carboxylate.

Step 4: A solution of phenyl chloroformate (0.11 mL, 918 μmol, 1.50 eq), tert-butyl 3-((3-amino-5-chlorophenoxy)methyl)pyrrolidine-1-carboxylate (200 mg, 612 μmol, 1.00 eq) and pyridine (0.15 mL, 1.84 mmol, 3.00 eq) in acetonitrile (2.00 mL) was stirred at 25° C. for 12 h. The mixture was diluted with ethyl acetate (80.0 mL) and water (80.0 mL). The organic layer was separated, and the aqueous phase was extracted with ethyl acetate (3×50.0 mL). The combined organic layers were dried over sodium sulfate, filtered, and concentrated under reduced pressure to give a residue. The residue was purified by silica gel column chromatography (petroleum ether/ethyl acetate=3/1) to afford tert-butyl 3-((3-chloro-5-((phenoxycarbonyl)amino)phenoxy) methyl)pyrrolidine-1-carboxylate.

Step 5: A solution of 3-(6-(hydroxymethyl)-1-oxoisoindolin-2-yl)piperidine-2,6-dione VIII-B (80.0 mg, 292 μmol, 1.00 eq), tert-butyl 3-((3-chloro-5-((phenoxycarbonyl)amino)phenoxy)methyl) pyrrolidine-1-carboxylate (156 mg, 350 μmol, 1.20 eq) and sodium hydride (60% dispersion in mineral oil) (23.3 mg, 583 μmol, 2.00 eq) in dimethylformamide (2.00 mL) was stirred at 0° C. for 4 h. The mixture was quenched by addition by hydrochloric acid (1M, 5.00 mL) and the filtrate was concentrated under reduced pressure to afford tert-butyl 3-((3-chloro-5-((((2-(2,6-dioxopiperidin-3-yl)-3-oxoisoindolin-5-yl)methoxy)carbonyl)amino)phenoxy)methyl)pyrrolidine-1-carboxylate.

Step 6: A solution of tert-butyl 3-((3-chloro-5-((((2-(2,6-dioxopiperidin-3-yl)-3-oxoisoindolin-5-yl)methoxy)carbonyl)amino)phenoxy)methyl)pyrrolidine-1-carboxylate (200 mg, 319 μmol, 1.00 eq) and hydrochloric acid (12 M, 26.6 μL, 1.00 eq) in water (2.00 mL) was stirred at 25° C. for 12 h. The mixture was diluted with acetonitrile (2.00 mL) and filtered. The filtrate was purified by a standard method to afford Compound 216. ¹H NMR (400 MHz, DMSO-d₆) δ=10.99 (s, 1H), 10.04 (s, 1H), 9.12 (br d, J=2.7 Hz, 2H), 7.79 (s, 1H), 7.71-7.61 (m, 2H), 7.15-7.09 (m, 2H), 6.70 (t, J=2.0 Hz, 1H), 5.28 (s, 2H), 5.16-5.08 (m, 1H), 4.52-4.43 (m, 1H), 4.38-4.31 (m, 1H), 4.03-3.93 (m, 2H), 3.30-3.10 (m, 3H), 3.05-2.84 (m, 2H), 2.78-2.65 (m, 1H), 2.60 (br d, J=16.8 Hz, 1H), 2.47-2.35 (m, 1H), 2.13-1.96 (m, 2H), 1.80-1.68 (m, 1H). MS (ESI) m/z 527.3 [M+H]⁺

### Compound 217:

Step 1: To a solution of 2-(2,6-dioxo-1-((2-(trimethylsilyl)ethoxy)methyl)piperidin-3-yl)-3-oxoisoindoline-5-carbaldehyde V (750 mg, 1.86 mmol, 1.00 eq) in dichloromethane (10.0 mL) was added methylmagnesium bromide 3.00 M in diethyl ether (3.00 M, 0.75 mL, 1.20 eq) dropwise at −78° C. The reaction was stirred at −78° C. for 2 h. The reaction was quenched with ammonium chloride (50.0 mL) to reach pH=7, and the mixture was extracted with ethyl acetate (3×30.0 mL). The combined organic layers were washed with brine (20.0 mL), dried over anhydrous sodium sulfate, filtered, and concentrated under reduces pressure to give a residue. The residue was purified by silica gel column chromatography (petroleum ether/ethyl acetate=10/1 to 2/3) to afford 3-(6-(1-hydroxyethyl)-1-oxoisoindolin-2-yl)-1-((2-(trimethylsilyl) ethoxy)methyl)piperidine-2,6-dione.

Step 2: A solution of 3-(6-(1-hydroxyethyl)-1-oxoisoindolin-2-yl)-1-((2-(trimethylsilyl)ethoxy) methyl)piperidine-2,6-dione (600 mg, 1.43 mmol, 1.00 eq) in hydrochloric acid/dioxane (6 M, 6.67 mL, 27.9 eq) was stirred at 50° C. for 1 h. The mixture was concentrated under reduced pressure to afford 3-(6-(1-hydroxyethyl)-1-oxoisoindolin-2-yl)-1-(hydroxymethyl)piperidine-2,6-dione.

Step 3: To a solution of 3-(6-(1-hydroxyethyl)-1-oxoisoindolin-2-yl)-1-(hydroxymethyl) piperidine-2,6-dione (450 mg, 1.41 mmol, 1.00 eq) in acetonitrile (10.0 mL) was added ammonium hydroxide 30% (0.20 mL, 1.56 mmol, 1.10 eq) dropwise. The reaction was stirred at 25° C. for 0.5 h. The reaction was quenched with 1M HCl to reach pH=3 and concentrated under reduced pressure to give a residue. The residue was purified by reversed phase preparative HPLC to afford 3-(6-(1-hydroxyethyl)-1-oxoisoindolin-2-yl)piperidine-2,6-dione.

Step 4: To a solution of 3-(6-(1-hydroxyethyl)-1-oxoisoindolin-2-yl)piperidine-2,6-dione (85.0 mg, 295 μmol, 1.00 eq) and phenyl (3-chloro-4-methylphenyl)carbamate (described in example 1) (84.9 mg, 324 μmol, 1.10 eq) in dimethylformamide (3.00 mL) was added sodium hydride (60% dispersion in mineral oil) (23.6 mg, 590 μmol, 2.00 eq) in portions at 0° C. The reaction was stirred at 0° C. for 0.5 h. The reaction was quenched with 1M hydrochloric acid (2.00 mL), filtered, and concentrated to give a residue. The residue was purified by a standard method to afford Compound 217. ¹H NMR (400 MHz, DMSO-d₆) δ=11.00 (br s, 1H), 9.93 (br s, 1H), 7.77 (s, 1H), 7.71-7.60 (m, 2H), 7.58 (d, J=1.6 Hz, 1H), 7.35-7.18 (m, 2H), 5.91 (q, J=6.4 Hz, 1H), 5.13 (dd, J=5.0, 13.3 Hz, 1H), 4.56-4.26 (m, 2H), 2.98-2.86 (m, 1H), 2.65-2.56 (m, 1H), 2.45-2.35 (m, 1H), 2.24 (s, 3H), 2.06-1.96 (m, 1H), 1.58 (d, J=6.6 Hz, 3H). MS (ESI) m/z 456.1 [M+H]⁺

Compound 218: General procedure B with variant i) was used for the preparation from compound VIII-B employing phenyl (2,3-dihydrobenzofuran-6-yl)carbamate.

¹H NMR (400 MHz, DMSO-d₆) δ=10.99 (s, 1H), 9.72 (br s, 1H), 7.79 (s, 1H), 7.69-7.61 (m, 2H), 7.09 (d, J=8.0 Hz, 1H), 6.98 (s, 1H), 6.88 (br d, J=7.9 Hz, 1H), 5.25 (s, 2H), 5.13 (dd, J=5.1, 13.2 Hz, 1H), 4.54-4.43 (m, 3H), 4.38-4.30 (m, 1H), 3.08 (t, J=8.6 Hz, 2H), 2.98-2.85 (m, 1H), 2.63-2.57 (m, 1H), 2.43-2.33 (m, 1H), 2.06-1.96 (m, 1H). MS (ESI) m/z 436.2 [M+H]⁺

To a solution of 2,3-dihydrobenzofuran-6-amine (200 mg, 1.48 mmol, 1.00 eq) in acetonitrile (8.00 mL) were added pyridine (0.60 mL, 7.40 mmol, 5.00 eq) and phenyl chloroformate (0.37 mL, 2.96 mmol, 2.00 eq) at 0° C. in portions. The reaction was stirred at 25° C. for 1 h. The mixture was concentrated and the obtained residue was purified by standard methods to afford phenyl (2,3-dihydrobenzofuran-6-yl)carbamate.

Compound 219: General procedure B with variant i) was used for the preparation from compound VIII-B employing phenyl (3-chloro-5-(difluoromethoxy)phenyl)carbamate.

¹H NMR (400 MHz, DMSO-d₆) δ=10.99 (s, 1H), 10.23 (s, 1H), 7.79 (s, 1H), 7.72-7.61 (m, 2H), 7.42 (t, J=1.8 Hz, 1H), 7.31 (s, 1H), 7.06 (t, J=44 Hz, 1H), 6.94 (t, J=2.0 Hz, 1H), 5.29 (s, 2H), 5.12 (dd, J=5.1, 13.3 Hz, 1H), 4.51-4.43 (m, 1H), 4.38-4.30 (m, 1H), 2.97-2.85 (m, 1H), 2.60 (br d, J=17.5 Hz, 1H), 2.40 (dd, J=4.4, 13.1 Hz, 1H), 2.06-1.96 (m, 1H). MS (ESI) m/z 494.1 [M+H]⁺

Step 1: To a solution of 3-chloro-5-nitro-phenol (1.50 g, 8.64 mmol, 1.00 eq) and sodium 2-chloro-2,2-difluoroacetate (5.27 g, 34.6 mmol, 4.00 eq) in dimethylformamide (17.0 mL) and water (2.00 mL) was added potassium carbonate (2.39 g, 17.3 mmol, 2.00 eq). The reaction was stirred at 100° C. for 12 h. The mixture was filtered and concentrated under reduced pressure to give a residue. The residue was diluted with water (100 mL) and extracted with ethyl acetate (3×20.0 mL). The combined organic layers were washed with saturated sodium carbonate solution (50.0 mL) and brine (20.0 mL), dried over anhydrous sodium sulfate, filtered, and concentrated under reduced pressure to afford 1-chloro-3-(difluoromethoxy)-5-nitrobenzene.

Step 2: To a solution of 1-chloro-3-(difluoromethoxy)-5-nitro-benzene (1.70 g, 7.60 mmol, 1.00 eq) in methanol (10.0 mL) and water (10.0 mL) were added iron powder (2.12 g, 38.0 mmol, 5.00 eq) and ammonium chloride (2.03 g, 38.0 mmol, 5.00 eq). The reaction was stirred at 80° C. for 2 h. The mixture was concentrated under reduced pressure to give a residue. The residue was diluted with saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 3-chloro-5-(difluoromethoxy)aniline.

Step 3: To a solution 3-chloro-5-(difluoromethoxy)aniline (400 mg, 2.07 mmol, 1.00 eq) in acetonitrile (10.0 mL) were added pyridine (0.83 mL, 10.3 mmol, 5.00 eq) and phenyl chloroformate (0.39 mL, 3.10 mmol, 1.50 eq). The reaction was stirred at 25° C. for 1 h. The mixture was concentrated and the obtained residue was purified by standard methods to afford phenyl (3-chloro-5-(difluoromethoxy)phenyl)carbamate.

### Compound 220:

Step 1: To a solution of 3-(6-(hydroxymethyl)-1-oxoisoindolin-2-yl)piperidine-2,6-dione (120 mg, 437 μmol, 1.00 eq) and tert-butyl 3-(3-chloro-5-((phenoxycarbonyl)amino) phenoxy)pyrrolidine-1-carboxylate (284 mg, 656 μmol, 1.50 eq) in dimethylformamide (2.00 mL) was added sodium hydride (60% dispersion in mineral oil) (35.0 mg, 875 μmol, 2.00 eq). The reaction was stirred at 25° C. for 1 h.

Step 2: A solution of tert-butyl 3-(3-chloro-5-((((2-(2,6-dioxopiperidin-3-yl)-3-oxoisoindolin-5-yl)methoxy)carbonyl)amino)phenoxy)pyrrolidine-1-carboxylate (250 mg, 407 μmol, 1.00 eq) in hydrochloric acid (4 M, 5.00 mL, 49.0 eq) was stirred at 25° C. for 12 h. The mixture was concentrated under reduced pressure to give a residue. The residue was purified by a standard method to afford Compound 220. ¹H NMR (400 MHz, DMSO-d₆) δ=11.00 (s, 1H), 10.07 (s, 1H), 9.32-9.13 (m, 2H), 7.80 (s, 1H), 7.75-7.60 (m, 2H), 7.23-7.09 (m, 2H), 6.75 (t, J=2.0 Hz, 1H), 5.29 (s, 2H), 5.18-5.08 (m, 2H), 4.56-4.41 (m, 1H), 4.41-4.31 (m, 1H), 3.32-3.21 (m, 2H), 3.00-2.87 (m, 1H), 2.63 (br d, J=2.8 Hz, 2H), 2.43 (dt, J=4.5, 13.1 Hz, 2H), 2.25-2.10 (m, 2H), 2.06-1.97 (m, 1H). MS (ESI) m/z 513.0 [M+H]⁺

### Preparation of tert-butyl 3-(3-chloro-5-((phenoxycarbonyl)amino) phenoxy)pyrrolidine-1-carboxylate

Step 1: To a solution of 3-chloro-5-nitro-phenol (5.00 g, 28.8 mmol, 1.00 eq), tert-butyl 3-hydroxypyrrolidine-1-carboxylate (5.93 g, 31.7 mmol, 1.10 eq) and triphenylphosphine (8.31 g, 31.7 mmol, 1.10 eq) in tetrahydrofuran (2.00 mL) was added diisopropyl azodiformate (6.16 mL, 31.7 mmol, 1.10 eq) at 0° C. The reaction was stirred at 25° C. for 12 h. The mixture was concentrated under reduced pressure to give a residue. The residue was purified by reversed phase preparative HPLC to afford tert-butyl 3-(3-chloro-5-nitrophenoxy)pyrrolidine-1-carboxylate.

Step 2: A mixture of tert-butyl 3-(3-chloro-5-nitro-phenoxy)pyrrolidine-1-carboxylate (3.00 g, 8.75 mmol, 100 eq), iron powder (1.47 g, 26.2 mmol, 3.00 eq) and ammonium chloride (2.34 g, 43.8 mmol, 5.00 eq) in methanol (20.0 mL) and water (10.0 mL) was stirred at 80° C. for 2 h. The mixture was filtered, and the filtrate was concentrated under reduced pressure to give a residue. It was added to water (80.0 mL) and saturated sodium bicarbonate (40.0 mL) and stirred for 10 min. The aqueous layer was extracted with ethyl acetate (3×60.0 mL). The combined organic layers were washed with brine (100 mL), dried over anhydrous sodium sulfate, filtered, and concentrated under reduced pressure to afford tert-butyl 3-(3-amino-5-chlorophenoxy)pyrrolidine-1-carboxylate.

Compound 221: General procedure B with variant i) was used for the preparation from compound VIII-B employing phenyl (3-(8-oxa-3-azabicyclo[3.2.1]octan-3-ylmethyl)-5-chloro-4-methylphenyl)carbamate. ¹H NMR (400 MHz, DMSO-d₆) δ=11.05-10.93 (m, 1H), 9.95-9.84 (m, 1H), 7.79 (s, 1H), 7.71-7.61 (m, 2H), 7.51 (s, 1H), 7.33 (d, J=1.7 Hz, 1H), 5.27 (s, 2H), 5.12 (dd, J=5.2, 13.4 Hz, 1H), 4.51-4.42 (m, 1H), 4.39-4.29 (m, 1H), 4.21-4.15 (m, 2H), 2.95-2.86 (m, 1H), 2.60 (br dd, J=1.7, 16.8 Hz, 1H), 2.45 (br d, J=10.3 Hz, 4H), 2.41-2.36 (m, 1H), 2.29-2.25 (m, 3H), 2.19 (br d, J=9.8 Hz, 2H), 2.04-1.98 (m, 1H), 1.88-1.79 (m, 2H), 1.68 (br dd, J=4.1, 7.0 Hz, 2H). MS (ESI) m/z 567.2 [M+H]⁺

Step 1: To a solution of (3-chloro-2-methyl-5-nitro-phenyl)methanol (2.00 g, 9.92 mmol, 1.00 eq) in dichloromethane (30.0 mL) was added thionyl chloride (3.60 mL, 49.6 mmol, 5.00 eq) at 25° C. The reaction was stirred at 25° C. for 12 h. The reaction was quenched by addition of ice water (20.0 mL) at 0° C., and the aqueous layer was extracted with ethyl acetate (3×20.0 mL). The combined organic layers were washed with brine (30.0 mL), dried over anhydrous sodium sulfate, filtered, and concentrated under reduced pressure to afford 1-chloro-3-(chloromethyl)-2-methyl-5-nitrobenzene.

Step 2: To a solution of 1-chloro-3-(chloromethyl)-2-methyl-5-nitro-benzene (1.40 g, 6.36 mmol, 1.00 eq), 8-oxa-3-azabicyclo[3.2.1]octane (1.14 g, 7.63 mmol, 1.20 eq, HCl) and potassium carbonate (2.64 g, 19.1 mmol, 3.00 eq) in acetonitrile (20.0 mL) was added potassium iodide (106 mg, 0.64 mmol, 0.10 eq) at 25° C. The reaction was stirred at 80° C. for 12 h. The mixture was filtered and concentrated under reduced pressure to give a residue. The residue was purified by silica gel column chromatography (petroleum ether/ethyl acetate=10/1 to 2/1) to afford 3-(3-chloro-2-methyl-5-nitrobenzyl)-8-oxa-3-azabicyclo[3.2.1]octane.

Step 3: To a solution of 3-(3-chloro-2-methyl-5-nitrobenzyl)-8-oxa-3-azabicyclo[3.2.1]octane (1.00 g, 3.37 mmol, 1.00 eq) in methanol (20.0 mL) were added iron powder (941 mg, 16.9 mmol, 5.00 eq), ammonium chloride (901 mg, 16.9 mmol, 5.00 eq) and water (5.00 mL) at 25° C. The reaction was stirred at 80° C. for 2 h. The mixture was concentrated under reduced pressure to give a residue. Sodium bicarbonate (20.0 mL) was added, and the aqueous layer was extracted with ethyl acetate (3×20.0 mL). The combined organic layers were washed with brine (20.0 mL), dried over anhydrous sodium sulfate, filtered, and concentrated under reduced pressure to afford 3-(8-oxa-3-azabicyclo[3.2.1]octan-3-ylmethyl)-5-chloro-4-methylaniline.

Step 4: To a solution of 3-(8-oxa-3-azabicyclo[3.2.1]octan-3-ylmethyl)-5-chloro-4-methylaniline (200 mg, 749 μmol, 1.00 eq) in acetonitrile (5.00 mL) were added pyridine (0.30 mL, 3.75 mmol, 5.00 eq) and phenyl chloroformate (0.14 mL, 1.12 mmol, 1.50 eq). The reaction was stirred at 25° C. for 1 h. The mixture was concentrated and the obtained residue was purified by standard methods to afford phenyl (3-(8-oxa-3-azabicyclo[3.2.1]octan-3-ylmethyl)-5-chloro-4-methylphenyl)carbamate.

Compound 222: General procedure B with variant ii) was used for the preparation from compound VIII-B employing cyclopropylmethanamine. ¹H NMR (400 MHz, DMSO-d₆) δ=10.99 (s, 1H), 7.71 (s, 1H), 7.60 (s, 2H), 7.40 (br t, J=5.6 Hz, 1H), 5.16-5.06 (m, 3H), 4.49-4.42 (m, 1H), 4.36-4.29 (m, 1H), 2.96-2.85 (m, 3H), 2.65-2.56 (m, 1H), 2.43-2.37 (m, 1H), 2.06-1.96 (m, 1H), 0.96-0.85 (m, 1H), 0.43-0.35 (m, 2H), 0.19-0.09 (m, 2H). MS (ESI) m/z 372.1 [M+H]⁺

Compound 223: General procedure B with variant ii) was used for the preparation from compound VIII-B employing piperidine. ¹H NMR (400 MHz, DMSO-d₆) δ=11.06-10.92 (m, 1H), 7.69 (s, 1H), 7.64-7.54 (m, 2H), 5.17 (s, 2H), 5.11 (dd, J=5.1, 13.3 Hz, 1H), 4.51-4.41 (m, 1H), 4.38-4.28 (m, 1H), 3.37 (br s, 4H), 2.97-2.85 (m, 1H), 2.60 (br d, J=17.6 Hz, 1H), 2.40 (br dd, J=4.3, 13.0 Hz, 1H), 2.06-1.95 (m, 1H), 1.59-1.50 (m, 2H), 1.48-1.39 (m, 4H). MS (ESI) m/z 386.1 [M+H]⁺

Compound 224: General procedure B with variant i) was used for the preparation from compound VIII-B employing phenyl (3-(difluoromethoxy)-4-methylphenyl)carbamate. ¹H NMR (400 MHz, DMSO-d₆) δ=10.99 (br s, 1H), 9.92 (br s, 1H), 7.79 (s, 1H), 7.70-7.61 (m, 2H), 7.41 (s, 1H), 7.19 (s, 2H), 7.09 (t, J=74 Hz, 1H), 5.27 (s, 2H), 5.12 (dd, J=5.1, 13.2 Hz, 1H), 4.52-4.43 (m, 1H), 4.38-4.30 (m, 1H), 2.96-2.85 (m, 1H), 2.63-2.57 (m, 1H), 2.40 (br dd, J=4.3, 13.1 Hz, 1H), 2.15 (s, 3H), 2.04-1.96 (m, 1H). MS (ESI) m/z 474.1 [M+H]⁺

Step 1: To a solution of 2-methyl-5-nitrophenol (5.00 g, 32.7 mmol, 1.00 eq) and sodium 2-chloro-2,2-difluoroacetate (12.4 g, 81.6 mmol, 2.50 eq) in dimethylformamide (50.0 mL) was added caesium carbonate (21.3 g, 65.3 mmol, 2.00 eq) in portions. The reaction was stirred at 100° C. for 2 h. The mixture was diluted with water (800 mL) and extracted with ethyl acetate (3×100 mL). The combined organic layers were washed with brine (80.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 2-(difluoromethoxy)-1-methyl-4-nitrobenzene.

Step 2: To a solution of 2-(difluoromethoxy)-1-methyl-4-nitrobenzene (4.85 g, 23.8 mmol, 1.00 eq) and ammonium chloride (6.39 g, 119 mmol, 5.00 eq) in methanol (40.0 mL) and water (40.0 mL) was added iron powder (4.00 g, 71.6 mmol, 3.00 eq) in portions. The reaction was stirred at 80° C. for 2 h. The mixture was filtered, and the filtrate was concentrated under reduced pressure to give a residue. Water (100 mL) was added, and the mixture was extracted with ethyl acetate (3×50.0 mL). The combined organic layers were washed with brine (20 mL), dried over anhydrous sodium sulfate, filtered, and concentrated to afford 3-(difluoromethoxy)-4-methylaniline.

Step 3: To a solution of 3-(difluoromethoxy)-4-methylaniline (1.00 g, 5.78 mmol, 1.00 eq) and pyridine (1.40 mL, 17.3 mmol, 3.00 eq) in acetonitrile (10.0 mL) was added phenyl chloroformate (1.09 mL, 8.66 mmol, 1.50 eq) dropwise. The reaction was stirred at 25° C. for 12 h. The mixture was diluted with water (150 mL) and extracted with ethyl acetate (3×50.0 mL). The combined organic layers were washed with brine (20.0 mL), dried over anhydrous sodium sulfate, filtered, and concentrated and the obtained residue was purified by standard methods to afford phenyl (3-(difluoromethoxy)-4-methylphenyl)carbamate.

Compound 225: General procedure B with variant i) was used for the preparation from compound VIII-B employing phenyl (3-(difluoromethoxy)-4-methyl-5-(morpholinomethyl)phenyl)carbamate. ¹H NMR (400 MHz, DMSO-d₆) δ=10.99 (s, 1H), 9.89 (s, 1H), 8.21 (s, 1H), 7.78 (s, 1H), 7.70-7.60 (m, 2H), 7.35 (br s, 1H), 7.26 (d, J=1.8 Hz, 1H), 7.25-6.85 (m, 1H), 5.27 (s, 2H), 5.16-5.08 (m, 1H), 4.51-4.28 (m, 2H), 3.55 (br t, J=4.3 Hz, 4H), 3.39 (s, 2H), 2.98-2.84 (m, 1H), 2.65-2.56 (m, 1H), 2.45-2.38 (m, 1H), 2.35 (br s, 4H), 2.14 (s, 3H), 2.06-1.96 (m, 1H). MS (ESI) m/z 573.4 [M+H]⁺

Step 1: To a solution of 2-methyl-5-nitro-benzoic acid (10.0 g, 55.2 mmol, 1.00 eq) in sulfuric acid (20.0 mL) was added N-Iodosuccinimide (14.9 g, 66.3 mmol, 1.20 eq). The reaction was stirred at 60° C. for 2 h. The mixture was diluted with ice water (200 mL) and the resulting precipitate was collected by filtration. The filter cake was washed with water (100 mL) and dried under vacuum to afford 3-iodo-2-methyl-5-nitro-benzoic acid.

Step 2: To a solution of 3-iodo-2-methyl-5-nitro-benzoic acid (5.00 g, 16.3 mmol, 1.00 eq), copper iodide (310 mg, 1.63 mmol, 0.100 eq), and quinolin-8-ol (0.56 mL, 3.26 mmol, 0.200 eq) in water (3.00 mL) and dimethylsulfoxide (3.00 mL) was added potassium hydroxide (3.65 g, 65.1 mmol, 4.00 eq). The reaction was stirred at 100° C. for 12 h. The mixture was diluted with water (50.0 mL) and extracted with ethyl acetate (2×50.0 mL). The combined organic layers were washed with water (50.0 mL), dried over anhydrous sodium sulfate, filtered, and concentrated under reduced pressure to afford 3-hydroxy-2-methyl-5-nitro-benzoic acid.

Step 3: To a solution of 3-hydroxy-2-methyl-5-nitro-benzoic acid (3.20 g, 16.2 mmol, 1.00 eq) and morpholine (1.71 mL, 19.5 mmol, 1.20 eq) in dichloromethane (100 mL) were added triethylamine (2.26 mL, 16.2 mmol, 1.00 eq) and O-(7-azabenzotriazol-1-yl)-N,N,N,N-tetramethyluroniumhexafluorophosphate (7.41 g, 19.5 mmol, 1.20 eq) at 20° C. The reaction was stirred at 20° C. for 12 h. The mixture was concentrated under reduced pressure to afford a residue. The residue was diluted with water (100 mL) and extracted with ethyl acetate (100 mL). The organic layer was washed with water (100 mL), dried over anhydrous sodium sulfate, filtered, and the filtrate was concentrated under reduced pressure to give a residue. The residue was purified by silica gel column chromatography (petroleum ether/ethyl acetate=1/1 to 0/1) to afford (3-hydroxy-2-methyl-5-nitro-phenyl)-morpholino-methanone.

Step 4: A solution of (3-hydroxy-2-methyl-5-nitro-phenyl)-morpholino-methanone (2.00 g, 7.51 mmol, 1.00 eq), potassium carbonate (2.08 g, 15.0 mmol, 2.00 eq) and sodium 2-chloro-2,2-difluoroacetate (4.58 g, 30.0 mmol, 4.00 eq) in dimethylformamide (24.0 mL) and water (3.00 mL) was stirred at 100° C. for 12 h. The mixture was diluted with water (150 mL) and extracted with ethyl acetate (3×100 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=3/1) to afford (3-(difluoromethoxy)-2-methyl-5-nitrophenyl)(morpholino)methanone.

Step 5: To a solution of (3-(difluoromethoxy)-2-methyl-5-nitrophenyl)(morpholino)methanone (1.70 g, 5.38 mmol, 1.00 eq) in tetrahydrofuran (3.00 mL) was added borane dimethyl sulfide complex (10 M, 1.08 mL, 2.00 eq) dropwise at 0° C. under nitrogen atmosphere. The reaction was stirred at 60° C. for 4 h. The reaction was quenched by addition with methanol (5.00 mL) and the solvents were removed under reduced pressure to afford 4-(3-(difluoromethoxy)-2-methyl-5-nitrobenzyl)morpholine.

Step 6: A solution of 4-(3-(difluoromethoxy)-2-methyl-5-nitrobenzyl)morpholine (1.50 g, 4.96 mmol, 1.00 eq), iron powder (1.94 g, 34.7 mmol, 7.00 eq) and ammonium chloride (1.86 g, 34.7 mmol, 7.00 eq) in methanol (5.00 mL) and water (5.00 mL) was stirred at 80° C. for 2 h. The mixture was filtered, and the filtrate was concentrated under reduced pressure to afford 3-(difluoromethoxy)-4-methyl-5-(morpholinomethyl)aniline.

Step 7: To a solution of 3-(difluoromethoxy)-4-methyl-5-(morpholinomethyl)aniline (500 mg, 1.84 mmol, 1.00 eq) in acetonitrile (5.00 mL) were added pyridine (0.45 mL, 5.51 mmol, 3.00 eq) and phenyl chloroformate (0.28 mL, 2.20 mmol, 1.20 eq). The reaction was stirred at 25° C. for 2 h. The mixture was diluted with ethyl acetate (80.0 mL) and water (80.0 mL).

The organic layer was separated, and the aqueous phase was extracted with ethyl acetate (3×50.0 mL). The combined organic layers were dried over sodium sulfate, filtered, and concentrated and the obtained residue was purified by standard methods to afford phenyl (3-(difluoromethoxy)-4-methyl-5-(morpholinomethyl)phenyl) carbamate.

Compound 226: General procedure B with variant ii) was used for the preparation from compound VIII-B employing pyrrolidine. ¹H NMR (400 MHz, DMSO-d₆) δ=10.99 (s, 1H), 7.70 (s, 1H), 7.65-7.58 (m, 2H), 5.17 (s, 2H), 5.12 (dd, J=5.1, 13.3 Hz, 1H), 4.51-4.41 (m, 1H), 4.37-4.27 (m, 1H), 3.31-3.25 (m, 4H), 2.98-2.86 (m, 1H), 2.60 (br d, J=17.5 Hz, 1H), 2.43-2.32 (m, 1H), 2.05-1.97 (m, 1H), 1.86-1.76 (m, 4H). MS (ESI) m/z 372.2 [M+H]⁺

Compound 227: General procedure B with variant ii) was used for the preparation from compound VIII-B employing 3-methylbutan-1-amine. ¹H NMR (400 MHz, DMSO-d₆) δ=10.99 (s, 1H), 7.70 (s, 1H), 7.66-7.55 (m, 2H), 7.28 (br t, J=5.4 Hz, 1H), 5.22-5.06 (m, 3H), 4.52-4.42 (m, 1H), 4.38-4.28 (m, 1H), 3.06-2.98 (m, 2H), 2.97-2.86 (m, 1H), 2.64-2.57 (m, 1H), 2.44-2.36 (m, 1H), 2.06-1.97 (m, 1H), 1.63-1.50 (m, 1H), 1.30 (q, J=6.9 Hz, 2H), 0.86 (d, J=6.6 Hz, 6H). MS (ESI) m/z 388.2 [M+H]⁺

Compound 228: General procedure B with variant ii) was used for the preparation from compound VIII-B employing cyclohexanamine. ¹H NMR (400 MHz, DMSO-d₆) δ=11.00 (s, 1H), 7.72 (s, 1H), 7.61 (s, 2H), 7.25 (br d, J=7.9 Hz, 1H), 5.18-5.08 (m, 3H), 4.50-4.42 (m, 1H), 4.37-4.29 (m, 1H), 3.30-3.22 (m, 1H), 2.98-2.86 (m, 1H), 2.61 (br dd, J=2.1, 15.6 Hz, 1H), 2.44-2.34 (m, 1H), 2.06-1.95 (m, 1H), 1.76 (br d, J=11.7 Hz, 2H), 1.71-1.61 (m, 2H), 1.54 (br d, J=12.8 Hz, 1H), 1.31-1.07 (m, 5H). MS (ESI) m/z 400.2 [M+H]⁺

Compound 229: General procedure B with variant ii) was used for the preparation from compound VIII-B employing cyclopentanamine. ¹H NMR (400 MHz, DMSO-d₆) δ=10.98 (br s, 1H), 7.71 (s, 1H), 7.60 (s, 2H), 7.31 (br d, J=7.1 Hz, 1H), 5.19-5.07 (m, 3H), 4.50-4.41 (m, 1H), 4.37-4.28 (m, 1H), 3.85-3.74 (m, 1H), 2.98-2.85 (m, 1H), 2.65-2.57 (m, 1H), 2.43-2.34 (m, 1H), 2.05-1.95 (m, 1H), 1.83-1.71 (m, 2H), 1.65-1.54 (m, 2H), 1.52-1.35 (m, 4H). MS (ESI) m/z 386.1 [M+H]⁺

Compound 230: General procedure B with variant ii) was used for the preparation from compound VIII-B employing 3-phenoxypyrrolidine. ¹H NMR (400 MHz, DMSO-d₆) δ=11.00 (s, 1H), 7.77-7.68 (m, 1H), 7.68-7.56 (m, 2H), 7.37-7.20 (m, 2H), 7.05-6.84 (m, 3H), 5.30-5.08 (m, 3H), 5.07-4.98 (m, 1H), 4.54-4.40 (m, 1H), 4.38-4.27 (m, 1H), 3.72-3.57 (m, 1H), 3.55-3.41 (m, 3H), 2.97-2.87 (m, 1H), 2.65-2.58 (m, 1H), 2.41 (br dd, J=4.5, 12.8 Hz, 1H), 2.19-1.99 (m, 3H). MS (ESI) m/z 464.1 [M+H]⁺

Step 1: To a solution of tert-butyl 3-hydroxypyrrolidine-1-carboxylate (3.00 g, 16.0 mmol, 1.00 eq) and triethylamine (5.58 mL, 40.0 mmol, 2.50 eq) in dichloromethane (30.0 mL) was added methanesulfonyl chloride (1.74 mL, 22.4 mmol, 1.40 eq) dropwise at 0° C. The reaction was stirred at 25° C. for 2 h. The mixture was diluted with water (150 mL) and extracted with ethyl acetate (3×80.0 mL). The combined organic layers were washed with brine (60.0 mL), dried over sodium sulfate, filtered, and concentrated under reduced pressure to afford tert-butyl 3-((methylsulfonyl)oxy)pyrrolidine-1-carboxylate.

Step 2: To a solution of tert-butyl 3-((methylsulfonyl)oxy)pyrrolidine-1-carboxylate (1.00 g, 3.77 mmol, 1.00 eq) and phenol (0.40 mL, 4.52 mmol, 1.20 eq) in dimethylformamide (10.0 mL) was added caesium carbonate (3.68 g, 11.3 mmol, 3.00 eq) in one portion. The reaction was stirred at 80° C. for 12 h. The mixture was diluted with water (50.0 mL) and extracted with ethyl acetate (3×60.0 mL). The combined organic layers were washed with brine (20.0 mL), dried over sodium sulfate, filtered, and concentrated under reduced pressure to give a residue. The residue was purified by silica gel column chromatography (petroleum ether/ethyl acetate=20/1 to 5/1) and then by reversed phase preparative HPLC to afford tert-butyl 3-phenoxypyrrolidine-1-carboxylate.

Step 3: A solution of tert-butyl 3-phenoxypyrrolidine-1-carboxylate (460 mg, 1.75 mmol, 1.00 eq) in 4 M hydrochloric acid/dioxane (4.00 mL) was stirred at 25° C. for 1 h and concentrated to afford 3-phenoxypyrrolidine.

Compound 231: General procedure B with variant i) was used for the preparation from compound VIII-B employing phenyl (3-(difluoromethoxy)-5-fluorophenyl) carbamate. ¹H NMR (400 MHz, DMSO-d₆) δ=11.00 (br s, 1H), 10.25 (s, 1H), 7.80 (s, 1H), 7.72-7.62 (m, 2H), 7.24 (t, J=73.2 Hz, 1H), 7.23-7.19 (m, 1H), 7.16 (s, 1H), 6.76 (td, J=2.2, 9.7 Hz, 1H), 5.30 (s, 2H), 5.13 (dd, J=5.1, 13.3 Hz, 1H), 4.51-4.45 (m, 1H), 4.38-4.32 (m, 1H), 2.95-2.86 (m, 1H), 2.64-2.60 (m, 1H), 2.46-2.36 (m, 1H), 2.05-1.98 (m, 1H). MS (ESI) m/z 478.1 [M+H]⁺

Step 1: To a solution of methanol (3.00 mL, 74.1 mmol, 3.93 eq) in N-methyl-pyrrolidone (10.0 mL) was added sodium hydride (60% dispersion in mineral oil) (830 mg, 20.7 mmol, 1.10 eq). The reaction was stirred at 0° C. for 1 h, then 1,3-difluoro-5-nitro-benzene (3.00 g, 18.9 mmol, 1.00 eq) was added. The reaction was stirred at 25° C. for another 11 h. The reaction was quenched with 1M hydrochloric acid (40.0 mL) and extracted with ethyl acetate (3×30.0 mL). The combined organic layers were washed with brine (15.0 mL), dried over anhydrous sodium sulfate, filtered, and concentrated under reduced pressure give a residue. The residue was purified by silica gel column chromatography (petroleum ether/ethyl acetate=10/1 to 1/1) to afford 1-fluoro-3-methoxy-5-nitro-benzene.

Step 2: To a solution of 1-fluoro-3-methoxy-5-nitro-benzene (2.50 g, 14.6 mmol, 1.00 eq) in dichloromethane (15.0 mL) was added boron tribromide (11.0 g, 43.8 mmol, 4.22 mL, 3.00 eq) at −78° C. The reaction was stirred at −78° C. for 1 h, and then at 25° C. for 11 h. The reaction was quenched with methanol (30.0 mL) and concentrated under reduced pressure to give a residue. The residue was purified by silica gel column chromatography (petroleum ether/ethyl acetate=10/1 to 5/1) to afford 3-fluoro-5-nitro-phenol.

Step 3: A solution of 3-fluoro-5-nitro-phenol (0.500 g, 3.18 mmol, 1.00 eq), sodium 2-chloro-2,2-difluoro-acetate (1.46 g, 9.55 mmol, 3.00 eq) and potassium carbonate (879 mg, 6.37 mmol, 2.00 eq) in dimethylformamide (10.0 mL) and water (2.00 mL) was stirred at 100° C. for 12 h. The mixture was poured into water (20.0 mL). The aqueous phase was extracted with ethyl acetate (3×30 mL). The combined organic layers were washed with brine (30.0 mL), dried over anhydrous sodium sulfate, filtered, and concentrated under reduced pressure to give a residue. The residue was purified by silica gel column chromatography (petroleum ether/ethyl acetate=10/1 to 5/1) to afford 1-(difluoromethoxy)-3-fluoro-5-nitrobenzene.

Step 4: A mixture of 1-(difluoromethoxy)-3-fluoro-5-nitrobenzene (700 mg, 3.38 mmol, 1.00 eq), ferrous powder (566 mg, 10.1 mmol, 3.00 eq) and ammonium chloride (904 mg, 16.9 mmol, 5.00 eq) in methanol (6.00 mL) and water (3.00 mL) was stirred at 80° C. for 1 h. The mixture was filtered, and the filtrate was concentrated under reduced pressure to afford 3-(difluoromethoxy)-5-fluoroaniline.

Step 5: To a solution of 3-(difluoromethoxy)-5-fluoroaniline (300 mg, 1.69 mmol, 1.00 eq) in acetonitrile (20.0 mL) were added pyridine (0.68 mL, 8.42 mmol, 4.97 eq) and phenyl chloroformate (0.25 mL, 2.00 mmol, 1.18 eq) at 0° C. The reaction was stirred at 25° C. for 1 h. The mixture was concentrated and the obtained residue was purified by standard methods to afford phenyl (3-(difluoromethoxy)-5-fluorophenyl)carbamate.

Compound 232: General procedure B with variant i) was used for the preparation from compound VIII-B employing phenyl (3-(difluoromethoxy)-4-fluorophenyl) carbamate. ¹H NMR (400 MHz, DMSO-d₆) δ=11.00 (s, 1H), 10.04 (s, 1H), 7.80 (s, 1H), 7.71-7.67 (m, 1H), 7.67-7.63 (m, 1H), 7.58 (br d, J=6.6 Hz, 1H), 7.37-7.27 (m, 2H), 7.21 (t, J=73.2 Hz, 1H), 5.29 (s, 2H), 5.13 (br d, J=8.2 Hz, 1H), 4.46 (s, 1H), 4.40-4.30 (m, 1H), 2.92 (br d, J=1.6 Hz, 1H), 2.64-2.59 (m, 1H), 2.46-2.37 (m, 1H), 2.06-1.98 (m, 1H). MS (ESI) m/z 478.1 [M+H]⁺

Step 1: To a solution of 2-fluoro-5-nitrophenol (500 mg, 3.18 mmol, 1.00 eq) and methyl 2-chloro-2,2-difluoroacetate (2.43 g, 15.9 mmol, 5.00 eq) in dimethylformamide (20.0 mL) and water (3.00 mL) was added potassium carbonate (880 mg, 6.37 mmol, 2.00 eq). The reaction was stirred at 100° C. for 12 h. Water (50.0 mL) was added, and the aqueous layer was extracted with ethyl acetate (3×20.0 mL). The organic layers were gathered, dried over anhydrous sodium sulfate, filtered, and concentrated under reduced pressure to give a residue. The residue was purified by silica gel column chromatography (petroleum ether/ethyl acetate=20/1 to 10/1) to afford 2-(difluoromethoxy)-1-fluoro-4-nitrobenzene.

Step 2: To a solution of 2-(difluoromethoxy)-1-fluoro-4-nitrobenzene (400 mg, 1.93 mmol, 1.00 eq) in methanol (9.00 mL) and water (3.00 mL) were added ammonium chloride (516 mg, 9.66 mmol, 5.00 eq) and ferrous powder (539 mg, 9.66 mmol, 5.00 eq). The reaction was stirred at 80° C. for 2 h. The mixture was filtered and concentrated under reduced pressure to afford 3-(difluoromethoxy)-4-fluoroaniline.

Step 3: To a solution of 3-(difluoromethoxy)-4-fluoroaniline (110 mg, 621 μmol, 1.00 eq) in acetonitrile (2.00 mL) were added pyridine (0.25 mL, 3.11 mmol, 5.00 eq) and phenyl chloroformate (0.08 mL, 0.68 mmol, 1.10 eq) at 0° C. The reaction was stirred at 0° C. for 0.5 h. The mixture was filtered, and the filtrate was concentrated and the obtained residue was purified by standard methods to afford phenyl (3-(difluoromethoxy)-4-fluorophenyl)carbamate.

Compound 233: General procedure B with variant i) was used for the preparation from compound VIII-B employing phenyl (3-ethoxy-4-methylphenyl) carbamate. ¹H NMR (400 MHz, DMSO-d₆) δ=10.99 (br s, 1H), 9.68 (br s, 1H), 7.79 (s, 1H), 7.69-7.65 (m, 1H), 7.65-7.61 (m, 1H), 7.14 (br s, 1H), 7.00 (d, J=8.2 Hz, 1H), 6.90 (br d, J=7.9 Hz, 1H), 5.25 (s, 2H), 5.12 (dd, J=5.1, 13.3 Hz, 1H), 4.50-4.43 (m, 1H), 4.38-4.29 (m, 1H), 3.95 (q, J=6.8 Hz, 2H), 2.96-2.86 (m, 1H), 2.63-2.56 (m, 1H), 2.40 (br dd, J=4.5, 13.1 Hz, 1H), 2.07-2.05 (m, 3H), 2.01 (dt, J=2.1, 6.2 Hz, 1H), 1.33 (t, J=6.9 Hz, 3H). MS (ESI) m/z 452.2 [M+H]⁺

Step 1: To a solution of 2-methyl-5-nitrophenol (1.00 g, 6.53 mmol, 1.00 eq) and iodoethane (0.57 mL, 7.18 mmol, 1.10 eq) in dimethylformamide (10.0 mL) was added potassium carbonate (2.71 g, 19.6 mmol, 3.00 eq) under nitrogen. The reaction was stirred at 40° C. for 2 h under nitrogen atmosphere. The mixture was poured into water (20.0 mL) and extracted with ethyl acetate (3×20.0 mL). The combined organic layers were gathered, washed with brine (10.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 2-ethoxy-1-methyl-4-nitrobenzene.

Step 2: To a solution of 2-ethoxy-1-methyl-4-nitrobenzene (0.900 g, 4.97 mmol, 1.00 eq) in methanol (10.0 mL) and water (10.0 mL) were added iron powder (1.39 g, 24.8 mmol, 5.00 eq) and ammonium chloride (1.33 g, 24.8 mmol, 5.00 eq). The reaction was stirred at 80° C. for 2 h. The mixture was concentrated under reduced pressure to give a slurry. The slurry was poured into saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 3-ethoxy-4-methylaniline.

Step 3: To a solution of 3-ethoxy-4-methylaniline (500 mg, 3.31 mmol, 1.00 eq) in acetonitrile (5.00 mL) were added pyridine (1.33 mL, 16.5 mmol, 5.00 eq) and phenyl chloroformate (0.50 mL, 3.97 mmol, 1.20 eq) at 0° C. The reaction was stirred at 25° C. for 1 h. The mixture was concentrated and the obtained residue was purified by standard methods to afford phenyl (3-ethoxy-4-methylphenyl)carbamate.

Compound 234: General procedure B with variant i) was used for the preparation from compound VIII-B employing phenyl (4-methyl-3-(morpholinomethyl)-5-(trifluoromethoxy)phenyl) carbamate. ¹H NMR (400 MHz, DMSO-d₆) δ=11.00 (s, 1H), 10.26 (br s, 1H), 10.20 (br s, 1H), 7.80 (s, 1H), 7.74-7.62 (m, 3H), 7.58 (br s, 1H), 5.31 (s, 2H), 5.12 (dd, J=5.1, 13.3 Hz, 1H), 4.52-4.30 (m, 4H), 3.94 (br d, J=12.1 Hz, 2H), 3.75 (br t, J=11.6 Hz, 2H), 3.30-3.15 (m, 3H), 2.99-2.84 (m, 1H), 2.61 (br d, J=17.0 Hz, 1H), 2.43 (br dd, J=8.7, 13.3 Hz, 2H), 2.35-2.30 (m, 3H), 2.07-1.98 (m, 1H). MS (ESI) m/z 591.1 [M+H]⁺

Step 1: To a solution of 2-methyl-5-nitro-benzoic acid (10.0 g, 55.2 mmol, 1.00 eq) in sulfuric acid (20.0 mL) was added N-Iodosuccinimide (14.9 g, 66.3 mmol, 1.20 eq). The reaction was stirred at 60° C. for 2 h. The mixture was diluted with ice water (200 mL) and filtered. The filter cake was washed with water (100 mL) and dried under vacuum to afford 3-iodo-2-methyl-5-nitro-benzoic acid.

Step 2: To a solution of 3-iodo-2-methyl-5-nitro-benzoic acid (5.00 g, 16.3 mmol, 1.00 eq), copper iodide (310 mg, 1.63 mmol, 0.10 eq) and quinolin-8-ol (563 μL, 3.26 mmol, 0.20 eq) in water (3.00 mL) and dimethylsulfoxide (3.00 mL) was added a solution of potassium hydroxide (3.65 g, 65.1 mmol, 4.00 eq). The reaction was stirred at 100° C. for 12 h. The mixture was diluted with water (50.0 mL) and extracted with ethyl acetate (2×50.0 mL). The combined organic layers were washed with water (50.0 mL), dried over anhydrous sodium sulfate, filtered, and concentrated under reduced pressure to afford 3-hydroxy-2-methyl-5-nitro-benzoic acid.

Step 3: To a solution of 3-hydroxy-2-methyl-5-nitro-benzoic acid (3.20 g, 16.2 mmol, 1.00 eq) and morpholine (1.71 mL, 19.5 mmol, 1.20 eq) in dichloromethane (100 mL) were added triethylamine (2.26 mL, 16.2 mmol, 1.00 eq) and O-(7-azabenzotriazol-1-yl)-N,N,N,N-tetramethyluroniumhexafluorophosphate (7.41 g, 19.5 mmol, 1.20 eq) at 20° C. The reaction was stirred at 20° C. for 12 h. The mixture was concentrated under reduced pressure to give a residue. The residue was diluted with water (100 mL) and extracted with ethyl acetate (100 mL). The organic layer was washed with water (100 mL), dried over anhydrous sodium sulfate, filtered, and the filtrate was concentrated under reduced pressure to give a residue. The residue was purified by silica gel column chromatography (petroleum ether/ethyl acetate=1/1 to 0/1) to afford (3-hydroxy-2-methyl-5-nitro-phenyl)-morpholino-methanone.

Step 4: To a solution of (3-hydroxy-2-methyl-5-nitro-phenyl)-morpholino-methanone (1.30 g, 4.88 mmol, 1.00 eq), silver trifluoromethanesulfonate (6.27 g, 24.4 mmol, 5.00 eq), 1-(chloromethyl)-4-fluoro-1,4-diazoniabicyclo[2.2.2]octane bis(tetrafluoroborate) (3.46 g, 9.77 mmol, 2.00 eq), N-fluorobenzenesulfonimide (3.08 g, 9.77 mmol, 2.00 eq) and caesium fluoride (4.45 g, 29.3 mmol, 1.08 mL, 6.00 eq) in toluene (130 mL) were added trimethyl(trifluoromethyl)silane (3.47 g, 24.4 mmol, 5.00 eq) and 2-fluoropyridine (2.10 mL, 24.4 mmol, 5.00 eq) under nitrogen. The reaction was stirred at 20° C. for 12 h under nitrogen. The mixture was filtered, and the filtrate was concentrated under reduced pressure to give a residue. The residue was diluted with water (100 mL) and extracted with ethyl acetate (100 mL). The organic layer was washed with water (20.0 mL), dried over anhydrous sodium sulfate, filtered, and the filtrate was concentrated under reduced pressure to give a residue. The residue was purified by silica gel column chromatography (petroleum ether/ethyl acetate=1/0 to 2/1) to afford (2-methyl-5-nitro-3-(trifluoromethoxy)phenyl)(morpholino)methanone.

Step 5: To a solution of (2-methyl-5-nitro-3-(trifluoromethoxy)phenyl)-morpholino-methanone (900 mg, 2.69 mmol, 1.00 eq) in tetrahydrofuran (15.0 mL) was added borane dimethyl sulfide complex (10.0 M, 539 μL, 2.00 eq) at 0° C. The reaction was stirred at 60° C. for 30 min. The mixture was quenched with methanol (2.00 mL) and concentrated under reduced pressure to give a residue. The residue was purified by reversed phase preparative HPLC to afford 4-(2-methyl-5-nitro-3-(trifluoromethoxy)benzyl)morpholine.

Step 6: To a solution of 4-(2-methyl-5-nitro-3-(trifluoromethoxy)benzyl)morpholine (400 mg, 1.25 mmol, 1.00 eq) in methanol (5.00 mL) and water (5.00 mL) was added iron powder (488 mg, 8.74 mmol, 7.00 eq) and ammonium chloride (468 mg, 8.74 mmol, 7.00 eq). The reaction was stirred at 80° C. for 2 h. The mixture was diluted with saturated sodium carbonate (1.00 mL) and extracted with ethyl acetate (2×10.0 mL). The combined organic layers were washed with water (5.00 mL), dried over anhydrous sodium sulfate, filtered, and the filtrate was concentrated under reduced pressure to give a residue. The residue was purified by silica gel column chromatography (petroleum ether/ethyl acetate=3/1 to 1/1) to afford 4-methyl-3-(morpholinomethyl)-5-(trifluoromethoxy)aniline.

Step 7: To a solution of 4-methyl-3-(morpholinomethyl)-5-(trifluoromethoxy)aniline (100 mg, 344 μmol, 1.00 eq) and potassium carbonate (57.1 mg, 413 μmol, 1.20 eq) in acetone (1.00 mL) was added phenyl chloroformate (47 μL, 379 μ, 1.10 eq) at 25° C. The reaction was stirred at 25° C. for 1 h. The mixture was diluted with water (6.00 mL) and extracted with ethyl acetate (10.0 mL). The organic layer was dried over anhydrous sodium sulfate, filtered, and the filtrate was concentrated to afford phenyl (4-methyl-3-(morpholinomethyl)-5-(trifluoromethoxy)phenyl)carbamate.

Compound 235: General procedure B with variant ii) was used for the preparation from compound VIII-B employing 1-(pyridin-2-yl)piperidin-4-amine. ¹H NMR (400 MHz, DMSO-d₆) δ=11.00 (s, 1H), 8.35-8.27 (m, 1H), 8.09 (dd, J=1.4, 4.9 Hz, 1H), 7.72 (s, 1H), 7.61 (s, 2H), 7.53-7.47 (m, 1H), 7.36 (br d, J=7.5 Hz, 1H), 6.83 (d, J=8.4 Hz, 1H), 6.59 (dd, J=5.0, 6.8 Hz, 1H), 5.17-5.08 (m, 3H), 4.50-4.43 (m, 1H), 4.37-4.30 (m, 1H), 4.20 (br d, J=13.1 Hz, 2H), 3.58 (br s, 1H), 2.92 (br s, 3H), 2.63-2.58 (m, 1H), 2.43-2.38 (m, 1H), 2.04-1.98 (m, 1H), 1.80 (br d, J=10.3 Hz, 2H), 1.41-1.30 (m, 2H). MS (ESI) m/z 478.2 [M+H]⁺

Step 1: A solution of 2-fluoropyridine (1.79 mL, 20.8 mmol, 1.00 eq), tert-butyl piperidin-4-ylcarbamate (5.00 g, 25.0 mmol, 1.20 eq) and potassium carbonate (5.75 g, 41.6 mmol, 2.00 eq) in dimethylacetamide (20.0 mL) was stirred at 120° C. for 12 h. The mixture was poured into water (20.0 mL) and extracted with ethyl acetate (3×20.0 mL). The combined organic layers were washed with brine (10.0 mL), dried over anhydrous sodium sulfate, filtered, and concentrated under reduced pressure to give a residue. The residue was purified by silica gel column chromatography (petroleum ether/ethyl acetate=5/1 to 3/1) to afford tert-butyl (1-(pyridin-2-yl)piperidin-4-yl)carbamate.

Step 2: A solution of tert-butyl (1-(pyridin-2-yl)piperidin-4-yl)carbamate (1.27 g, 4.58 mmol, 1.00 eq) and hydrochloric acid/ethyl acetate (8.00 mL) in ethyl acetate (24.0 mL) was stirred at 25° C. for 2 h. The mixture was concentrated and the obtained residue was purified by standard methods and filtered to afford 1-(pyridin-2-yl)piperidin-4-amine.

Compound 236: General procedure B with variant ii) was used for the preparation from compound VIII-B employing 1-phenylpiperazine. ¹H NMR (400 MHz, DMSO-d₆) δ=11.00 (s, 1H), 7.74 (s, 1H), 7.69-7.59 (m, 2H), 7.27-7.18 (m, 2H), 6.95 (d, J=7.9 Hz, 2H), 6.81 (t, J=7.3 Hz, 1H), 5.22 (s, 2H), 5.12 (dd, J=5.1, 13.3 Hz, 1H), 4.52-4.42 (m, 1H), 4.39-4.29 (m, 1H), 3.55 (br s, 4H), 3.17-3.09 (m, 4H), 2.92 (ddd, J=5.4, 13.7, 17.4 Hz, 1H), 2.63-2.58 (m, 1H), 2.44-2.35 (m, 1H), 2.06-1.98 (m, 1H). MS (ESI) m/z 463.2 [M+H]⁺

Compound 237: General procedure B with variant i) was used for the preparation from compound VIII-B employing phenyl (3-methoxy-4-methylphenyl)carbamate. ¹H NMR (400 MHz, DMSO-d₆) δ=11.00 (br s, 1H), 9.71 (br s, 1H), 7.80 (s, 1H), 7.71-7.67 (m, 1H), 7.66-7.61 (m, 1H), 7.17 (s, 1H), 7.01 (d, J=8.2 Hz, 1H), 6.93 (s, 1H), 5.27 (s, 2H), 5.16-5.10 (m, 1H), 4.46 (s, 1H), 4.37 (s, 1H), 3.73 (s, 3H), 2.97-2.86 (m, 1H), 2.58 (br s, 1H), 2.43-2.35 (m, 1H), 2.07 (s, 3H), 2.03 (br s, 1H). MS (ESI) m/z 438.1 [M+H]⁺

To a solution of 3-methoxy-4-methylaniline (500 mg, 3.64 mmol, 1.00 eq) in acetonitrile (5.00 mL) were added pyridine (1.47 mL, 18.2 mmol, 5.00 eq) and phenyl chloroformate (0.91 mL, 7.29 mmol, 2.00 eq) at 0° C. The reaction was stirred at 0° C. for 1 h. The mixture was concentrated under reduced pressure to give a residue. The residue was purified by reversed phase preparative HPLC. The desired fraction was collected and concentrated under reduced pressure to give concentrated solution. The solution was diluted with water/ethyl acetate (40.0 ml/80.0 ml). The organic layer was separated, dried over anhydrous sodium sulfate, filtered, and concentrated by standard methods to afford phenyl (3-methoxy-4-methylphenyl)carbamate.

Compound 238: General procedure B with variant i) was used for the preparation from compound VIII-B employing phenyl (4-chloro-2-methoxyphenyl)carbamate. ¹H NMR (400 MHz, DMSO-d₆) δ=11.08-10.95 (m, 1H), 8.86 (s, 1H), 7.81 (s, 1H), 7.71-7.61 (m, 3H), 7.10 (d, J=2.3 Hz, 1H), 6.98 (dd, J=2.3, 8.6 Hz, 1H), 5.26 (s, 2H), 5.18-5.10 (m, 1H), 4.51-4.44 (m, 1H), 4.39-4.30 (m, 1H), 3.83 (s, 3H), 2.97-2.88 (m, 1H), 2.62-2.59 (m, 1H), 2.41 (br dd, J=4.4, 12.9 Hz, 1H), 2.06-1.98 (m, 1H). MS (ESI) m/z 458.1 [M+H]⁺

To a solution of 4-chloro-2-methoxyaniline (1.00 g, 6.35 mmol, 1.00 eq) in acetonitrile (5.00 mL) were added pyridine (2.57 mL, 31.8 mmol, 5.01 eq) and phenyl chloroformate (0.88 mL 7.03 mmol, 1.11 eq) at 0° C. The reaction was stirred at 0° C. for 1 h. The mixture was concentrated and the obtained residue was purified by standard methods to afford phenyl (4-chloro-2-methoxyphenyl)carbamate.

Compound 239: General procedure B with variant i) was used for the preparation with from compound VIII-B employing phenyl (4-cyclopropylphenyl)carbamate. ¹H NMR (400 MHz, DMSO-d₆) δ=10.99 (s, 1H), 9.68 (br s, 1H), 7.78 (s, 1H), 7.71-7.59 (m, 2H), 7.34 (br d, J=8.4 Hz, 2H), 6.98 (d, J=8.6 Hz, 2H), 5.25 (s, 2H), 5.18-5.08 (m, 1H), 4.53-4.43 (m, 1H), 4.37-4.29 (m, 1H), 2.99-2.85 (m, 1H), 2.65-2.56 (m, 1H), 2.47-2.34 (m, 1H), 2.05-1.97 (m, 1H), 1.89-1.79 (m, 1H), 0.92-0.84 (m, 2H), 0.62-0.55 (m, 2H). MS (ESI) m/z 434.3 [M+H]⁺

To a solution of 4-cyclopropylaniline (500 mg, 3.75 mmol, 1.00 eq) in acetonitrile (5.00 mL) was added pyridine (0.91 mL, 11.3 mmol, 3.00 eq) and phenyl chloroformate (0.56 mL, 4.50 mmol, 1.20 eq). The reaction was stirred at 25° C. for 2 h. The mixture was diluted with ethyl acetate (50.0 mL) and water (50.0 mL). The organic layer was separated, and the aqueous phase was extracted with ethyl acetate (3×100 mL). The combined organic layers were dried over sodium sulfate, filtered, and concentrated and the obtained residue was purified by standard methods to afford phenyl (4-cyclopropylphenyl)carbamate.

Compound 240: General procedure B with variant i) was used for the preparation from compound VIII-B employing phenyl (3-chloro-5-((1-methylpyrrolidin-3-yl)methoxy)phenyl)carbamate. ¹H NMR (400 MHz, DMSO-d₆) δ=10.99 (s, 1H), 9.99 (s, 1H), 8.15 (s, 1H), 7.79 (s, 1H), 7.70-7.61 (m, 2H), 7.13 (s, 1H), 7.06 (s, 1H), 6.67 (t, J=1.9 Hz, 1H), 5.28 (s, 2H), 5.12 (dd, J=5.2, 13.1 Hz, 1H), 4.52-4.43 (m, 1H), 4.38-4.30 (m, 1H), 3.85 (br dd, J=4.7, 7.0 Hz, 2H), 2.96-2.88 (m, 1H), 2.74-2.68 (m, 1H), 2.62 (br s, 1H), 2.58 (br s, 2H), 2.55-2.52 (m, 2H), 2.43-2.38 (m, 1H), 2.35 (s, 3H), 2.04-1.91 (m, 2H), 1.53 (br dd, J=6.3, 12.5 Hz, 1H). MS (ESI) m/z 541.2 [M+H]⁺

Step 1: To a solution of tert-butyl 3-(hydroxymethyl)pyrrolidine-1-carboxylate (3.00 g, 14.9 mmol, 1.00 eq) and triethylamine (3.77 g, 37.3 mmol, 5.19 mL, 2.50 eq) in dichloromethane (30.0 mL) at 0° C. was added methylsulfamoyl chloride (1.50 mL, 19.4 mmol, 1.30 eq) dropwise under nitrogen atmosphere. The reaction was stirred at 25° C. for 2 h. The mixture was diluted with ethyl acetate (100 mL) and water (150 mL). The organic layer was separated, and the aqueous layer was extracted with ethyl acetate (3×100 mL). The organic layers were gathered, dried over sodium sulfate, filtered, and concentrated under reduced pressure to afford tert-butyl 3-(((methylsulfonyl)oxy)methyl)pyrrolidine-1-carboxylate.

Step 2: To a solution of tert-butyl 3-(((methylsulfonyl)oxy)methyl) pyrrolidine-1-carboxylate (3.00 g, 10.8 mmol, 1.00 eq) in dimethylformamide (30.0 mL) was added 3-chloro-5-nitrophenol (2.05 g, 11.8 mmol, 1.10 eq) and cesium carbonate (10.5 g, 32.2 mmol, 3.00 eq). The reaction was stirred at 80° C. for 12 h. The mixture was diluted with ethyl acetate (100 mL) and water (100 mL). The organic layer was separated, and the aqueous layer was extracted with ethyl acetate (3×80.0 mL). The organic layers were gathered, dried over sodium sulfate, filtered, and concentrated under reduced pressure to give a residue. The residue was purified by silica gel column chromatography (petroleum ether/ethyl acetate=3/1) to afford tert-butyl 3-((3-chloro-5-nitrophenoxy)methyl) pyrrolidine-1-carboxylate.

Step 3: To a solution of tert-butyl 3-((3-chloro-5-nitrophenoxy)methyl)pyrrolidine-1-carboxylate (1.10 g, 3.08 mmol, 1.00 eq) in ethyl acetate (5.00 mL) was added hydrochloric acid in ethyl acetate (4 M, 10 mL). The reaction was stirred at 25° C. for 2 h. The mixture was concentrated under reduced pressure to afford 3-((3-chloro-5-nitrophenoxy) methyl)pyrrolidine.

Step 4: To a solution of 3-((3-chloro-5-nitrophenoxy)methyl)pyrrolidine (1.50 g, 5.84 mmol, 1.00 eq) in 2,2,2-trifluoroethanol (10.0 mL) was added paraformaldehyde (0.80 mL, 29.2 mmol, 5.00 eq). The reaction was stirred at 60° C. for 0.5 h. Sodium borohydride (442 mg, 11.7 mmol, 2.00 eq) was added in portions, and the reaction was stirred at 60° C. for 1 h. The reaction was quenched with saturated ammonium chloride solution (10.0 mL) and concentrated under reduced pressure to give a residue. The residue was purified by reversed phase preparative HPLC to afford 3-((3-chloro-5-nitrophenoxy)methyl)-1-methylpyrrolidine.

Step 5: To a solution of 3-((3-chloro-5-nitrophenoxy)methyl)-1-methylpyrrolidine (1.20 g, 4.43 mmol, 1.00 eq) in methanol (6.00 mL) and water (6.00 mL) was added ammonium chloride (1.66 g, 31.0 mmol, 7.00 eq) and iron powder (1.73 g, 31.0 mmol, 7.00 eq). The reaction was stirred at 80° C. for 2 h. The mixture was filtered over Celite, and the filtrate was concentrated under reduced pressure to afford 3-chloro-5-((1-methylpyrrolidin-3-yl)methoxy)aniline.

Step 6: To a solution of phenyl chloroformate (0.28 mL, 2.24 mmol, 1.20 eq) in acetonitrile (5.00 mL) was added pyridine (0.45 mL, 5.61 mmol, 3.00 eq) and 3-chloro-5-((1-methylpyrrolidin-3-yl) methoxy)aniline (450 mg, 1.87 mmol, 1.00 eq). The reaction was stirred at 25° C. for 2 h. The mixture was concentrated and the obtained residue was purified by standard methods to afford phenyl (3-chloro-5-((1-methylpyrrolidin-3-yl) methoxy)phenyl)carbamate.

Compound 241: General procedure B with variant i) was used for the preparation from compound VIII-B employing phenyl (3-fluoro-4-methyl-5-(morpholinomethyl)phenyl)carbamate. ¹H NMR (400 MHz, DMSO-d₆) δ=10.99 (s, 1H), 10.90 (br s, 1H), 10.12 (s, 1H), 7.79 (s, 1H), 7.71-7.61 (m, 2H), 7.50 (s, 1H), 7.36 (br d, J=11.7 Hz, 1H), 5.29 (s, 2H), 5.11 (dd, J=5.1, 13.3 Hz, 1H), 4.51-4.43 (m, 1H), 4.38-4.29 (m, 3H), 3.90-3.81 (m, 4H), 3.34-3.14 (m, 4H), 2.97-2.86 (m, 1H), 2.60 (br d, J=17.7 Hz, 1H), 2.46-2.36 (m, 1H), 2.29 (d, J=1.7 Hz, 3H), 2.09-1.94 (m, 1H). MS (ESI) m/z 525.3 [M+H]⁺

Step 1: To a solution of 3-fluoro-2-methylbenzoic acid (10.0 g, 64.9 mmol, 1.00 eq) in sulfuric acid (100 mL) was added potassium nitrate (7.22 g, 71.4 mmol, 1.10 eq) in portions at 0° C. The reaction was stirred at 0° C. for 1 h. The mixture was poured into water (100 mL) and the resulting precipitate was collected by filtration. The filter cake was dried under vacuum, then added to water (100 mL) and extracted with ethyl acetate (3×80.0 mL). The combined organic layers were washed with brine (60.0 mL), dried over sodium sulfate, filtered, and concentrated under reduced pressure to afford 3-fluoro-2-methyl-5-nitrobenzoic acid.

Step 2: To a solution of 3-fluoro-2-methyl-5-nitrobenzoic acid (11.0 g, 55.2 mmol, 1.00 eq) in tetrahydrofuran (100 mL) was added borane dimethyl sulfide complex (10 M, 11.0 mL, 2.00 eq) at 0° C. The reaction was stirred at 25° C. for 12 h. The mixture was poured into methanol (200 mL) and concentrated under reduced pressure to give a residue. Water (150 mL) was added, and the pH was adjusted to pH=10 by addition of 15% sodium hydroxide solution. The aqueous layer was extracted with ethyl acetate (3×80.0 mL). The combined organic layers were washed with brine (50.0 mL), dried over sodium sulfate, filtered, and concentrated under reduced pressure to give a residue. The residue was purified by silica gel column chromatography (petroleum ether/ethyl acetate=4/1) to afford (3-fluoro-2-methyl-5-nitrophenyl)methanol.

Step 3: To a solution of (3-fluoro-2-methyl-5-nitrophenyl)methanol (870 mg, 4.70 mmol, 1.00 eq) in dichloromethane (10.0 mL) was added thionyl chloride (1.70 mL, 23.5 mmol, 5.00 eq) dropwise at 0° C. The reaction was stirred at 25° C. for 12 h. The mixture was concentrated under reduced pressure to afford 1-(chloromethyl)-3-fluoro-2-methyl-5-nitrobenzene.

Step 4: To a solution of 1-(chloromethyl)-3-fluoro-2-methyl-5-nitrobenzene (950 mg, 4.67 mmol, 1.00 eq) and triethylamine (1.62 mL, 11.7 mmol, 2.50 eq) in acetonitrile (10.0 mL) was added morpholine (0.51 mL, 5.83 mmol, 1.25 eq) dropwise. The reaction was stirred at 25° C. for 12 h. The mixture was concentrated under reduced pressure to give a residue. The residue was diluted with water (80.0 mL) and extracted with ethyl acetate (3×60.0 mL). The combined organic layers were washed with brine (40.0 mL), dried over sodium sulfate, filtered, and concentrated under reduced pressure to give a residue. The residue was purified by silica gel column chromatography (petroleum ether/ethyl acetate=4/1) to afford 4-(3-fluoro-2-methyl-5-nitrobenzyl)morpholine.

Step 5: To a solution of 4-(3-fluoro-2-methyl-5-nitrobenzyl)morpholine (1.00 g, 3.93 mmol, 1.00 eq) and ammonium chloride (1.05 g, 19.7 mmol, 5.00 eq) in methanol (8.00 mL) and water (2.00 mL) was added iron powder (1.10 g, 19.7 mmol, 5.00 eq) in portions. The reaction was stirred at 80° C. for 2 h. The mixture was filtered, and the filtrate was diluted with water (80.0 mL) and extracted with ethyl acetate (3×60.0 mL). The combined organic layers were washed with brine (40.0 mL), dried over sodium sulfate, filtered, and concentrated under reduced pressure to afford 3-fluoro-4-methyl-5-(morpholinomethyl)aniline.

Step 6: To a solution of 3-fluoro-4-methyl-5-(morpholinomethyl)aniline (300 mg, 1.34 mmol, 1.00 eq) and pyridine (0.54 mL, 6.69 mmol, 5.00 eq) in acetonitrile (5.00 mL) was added phenyl chloroformate (0.20 mL, 1.61 mmol, 1.20 eq) dropwise at 0° C. The reaction was stirred at 25° C. for 12 h. The mixture was diluted with water (50.0 mL) and extracted with ethyl acetate (3×40.0 mL). The combined organic layers were washed with brine (30.0 mL), dried over sodium sulfate, filtered, and concentrated and the obtained residue was purified by standard methods to afford phenyl (3-fluoro-4-methyl-5-(morpholinomethyl)phenyl)carbamate.

Compound 242: General procedure B with variant i) was used for the preparation from compound VIII-B employing phenyl (6-phenylpyridin-3-yl)carbamate. ¹H NMR (400 MHz, DMSO-d₆) δ=10.99 (s, 1H), 10.23 (br s, 1H), 8.75 (s, 1H), 8.06-7.95 (m, 4H), 7.82 (s, 1H), 7.73-7.68 (m, 1H), 7.67-7.62 (m, 1H), 7.51-7.45 (m, 2H), 7.44-7.39 (m, 1H), 5.33 (s, 2H), 5.13 (dd, J=5.1, 13.2 Hz, 1H), 4.51-4.45 (m, 1H), 4.38-4.32 (m, 1H), 2.97-2.86 (m, 1H), 2.60 (br d, J=17.2 Hz, 1H), 2.41 (br dd, J=4.4, 12.9 Hz, 1H), 2.05-1.97 (m, 1H). MS (ESI) m/z 471.2 [M+H]⁺

To a solution of 6-phenylpyridin-3-amine (300 mg, 1.76 mmol, 1.00 eq) and pyridine (0.71 mL, 8.81 mmol, 5.00 eq) in acetonitrile (5.00 mL) was added phenyl chloroformate (0.26 mL, 2.12 mmol, 1.20 eq) in portions at 0° C. The reaction was stirred at 25° C. for 1 h. The mixture was concentrated and the obtained residue was purified by standard methods to afford phenyl (6-phenylpyridin-3-yl)carbamate.

Compound 243: General procedure B with variant i) was used for the preparation from compound VIII-B employing phenyl (6-(tert-butyl)pyridin-3-yl)carbamate. ¹H NMR (400 MHz, DMSO-d₆) δ=11.01 (s, 1H), 10.80-10.56 (m, 1H), 8.91-8.80 (m, 1H), 8.29 (br d, J=8.5 Hz, 1H), 7.87 (br d, J=8.6 Hz, 1H), 7.82 (s, 1H), 7.73-7.69 (m, 1H), 7.68-7.63 (m, 1H), 5.35 (s, 2H), 5.13 (dd, J=5.1, 13.3 Hz, 1H), 4.53-4.43 (m, 1H), 4.38-4.32 (m, 1H), 2.97-2.87 (m, 1H), 2.66-2.57 (m, 1H), 2.47-2.35 (m, 1H), 2.06-1.97 (m, 1H), 1.45-1.37 (m, 9H). MS (ESI) m/z 451.2 [M+H]⁺

To a solution of 6-(tert-butyl)pyridin-3-amine (150 mg, 1.00 mmol, 1.00 eq) and pyridine (0.40 mL, 5.00 mmol, 5.00 eq) in acetonitrile (5.00 mL) was added phenyl chloroformate (0.15 mL, 1.20 mmol, 1.20 eq). The reaction was stirred at 25° C. for 1 h. The mixture was concentrated and the obtained residue was purified by standard methods to afford phenyl (6-(tert-butyl)pyridin-3-yl)carbamate.

### Compound 244:

Step 1: A solution of 3-chloro-2-methyl-5-nitrophenol (260 mg, 1.39 mmol, 1.00 eq), tert-butyl 3-((methylsulfonyl)oxy)pyrrolidine-1-carboxylate (441 mg, 1.66 mmol, 1.20 eq) and potassium carbonate (575 mg, 4.16 mmol, 3.00 eq) in dimethylformamide (10.0 mL) was stirred at 80° C. for 4 h. The mixture was poured into water (20.0 mL) and extracted with ethyl acetate (3×20.0 mL). The combined organic layers were washed with brine (10.0 mL), dried over anhydrous sodium sulfate, filtered, and concentrated under reduced pressure to give a residue. The residue was purified by silica gel column chromatography (petroleum ether/ethyl acetate=1/0 to 5/1) to afford tert-butyl 3-(3-chloro-2-methyl-5-nitrophenoxy)pyrrolidine-1-carboxylate.

Step 2: To a mixture of tert-butyl 3-(3-chloro-2-methyl-5-nitrophenoxy)pyrrolidine-1-carboxylate (430 mg, 1.21 mmol, 1.00 eq), iron powder (337 mg, 6.03 mmol, 5.00 eq) and ammonium chloride (322 g, 6.03 mmol, 5.00 eq) in methanol (10.0 mL) was added water (10.0 mL) at 25° C. The reaction was stirred at 80° C. for 2 h. The mixture was concentrated under reduced pressure to give a residue. The residue was added to saturated sodium bicarbonate (20.0 mL) and extracted with ethyl acetate (3×20.0 mL). The combined organic layers were washed with brine (20.0 mL), dried over anhydrous sodium sulfate, filtered, and concentrated under reduced pressure to afford tert-butyl 3-(5-amino-3-chloro-2-methylphenoxy)pyrrolidine-1-carboxylate.

Step 3: To a solution of tert-butyl 3-(5-amino-3-chloro-2-methylphenoxy)pyrrolidine-1-carboxylate (170 mg, 520 μmol, 1.00 eq) and pyridine (0.21 mL, 2.60 mmol, 5.00 eq) in acetonitrile (5.00 mL) was added phenyl chloroformate (78.0 μL, 623 μmol, 1.20 eq) in portions at 0° C. The reaction was stirred at 25° C. for 1 h. The mixture was concentrated under reduced pressure to give a residue, which was purified by reversed phase preparative HPLC to afford tert-butyl 3-(3-chloro-2-methyl-5-((phenoxycarbonyl)amino)phenoxy)pyrrolidine-1-carboxylate.

Step 4: To a solution of 3-(6-(hydroxymethyl)-1-oxoisoindolin-2-yl)piperidine-2,6-dione VIII-B (100 mg, 365 μmol, 1.00 eq) and tert-butyl 3-(3-chloro-2-methyl-5-((phenoxycarbonyl)amino)phenoxy)pyrrolidine-1-carboxylate (179 mg, 401 μmol, 1.10 eq) in dimethylformamide (3.00 mL) was added sodium hydride (60% dispersion in mineral oil) (30.0 mg, 750 μmol, 2.06 eq) in portions at 0° C. The reaction was stirred at 25° C. for 1 h. The reaction was quenched with hydrochloric acid (1 M, 0.50 mL), and the solvents were removed under reduced pressure to give a residue. The residue was purified by reversed phase preparative HPLC to afford tert-butyl 3-(3-chloro-5-((((2-(2,6-dioxopiperidin-3-yl)-3-oxoisoindolin-5-yl)methoxy)carbonyl)amino)-2-methylphenoxy)pyrrolidine-1-carboxylate.

Step 5: To a solution of tert-butyl 3-(3-chloro-5-((((2-(2,6-dioxopiperidin-3-yl)-3-oxoisoindolin-5-yl)methoxy)carbonyl)amino)-2-methylphenoxy)pyrrolidine-1-carboxylate (120 mg, 191 μmol, 1.00 eq) in ethyl acetate (10.0 mL) was added hydrochloric acid/ethyl acetate (4 M, 4.62 mL, 96.5 eq). The reaction was stirred at 25° C. for 1 h. The mixture was concentrated to give a residue, and purified by a standard method to afford Compound 244 (hydrochloride). ¹H NMR (400 MHz, DMSO-d₆) δ=10.99 (s, 1H), 9.95 (br s, 1H), 9.54 (br s, 1H), 9.49-9.38 (m, 1H), 7.79 (s, 1H), 7.70-7.62 (m, 2H), 7.25-7.11 (m, 2H), 5.28 (s, 2H), 5.12 (dd, J=5.1, 13.2 Hz, 1H), 5.02 (br s, 1H), 4.52-4.44 (m, 1H), 4.38-4.31 (m, 1H), 3.51-3.43 (m, 2H), 3.37-3.31 (m, 2H), 3.27 (br dd, J=6.9, 9.9 Hz, 1H), 2.97-2.85 (m, 1H), 2.60 (br d, J=16.9 Hz, 1H), 2.40 (br dd, J=4.5, 13.1 Hz, 1H), 2.23-2.16 (m, 1H), 2.14 (s, 3H), 2.06-1.95 (m, 1H). MS (ESI) m/z 527.2 [M+H]⁺

Compound 245: General procedure B with variant i) was used for the preparation with from compound VIII-B employing phenyl (3-chloro-5-ethyl-4-methylphenyl)carbamate. ¹H NMR (400 MHz, DMSO-d₆) δ=10.99 (s, 1H), 9.85 (s, 1H), 7.79 (s, 1H), 7.70-7.61 (m, 2H), 7.46 (s, 1H), 7.22 (d, J=1.9 Hz, 1H), 5.27 (s, 2H), 5.12 (dd, J=5.1, 13.3 Hz, 1H), 4.50-4.44 (m, 1H), 4.37-4.31 (m, 1H), 2.99-2.86 (m, 1H), 2.65-2.61 (m, 1H), 2.60-2.56 (m, 2H), 2.46-2.36 (m, 1H), 2.22 (s, 3H), 2.06-1.98 (m, 1H), 1.11 (t, J=7.5 Hz, 3H). MS (ESI) m/z 470.2 [M+H]⁺

Step 1: To a solution of 2-chloro-1-methyl-4-nitrobenzene (0.60 mL, 2.91 mmol, 1.00 eq) in sulfuric acid (5.00 mL, 98% purity) was added N-iodosuccinimide (787 mg, 3.50 mmol, 1.20 eq) in portions. The reaction was stirred at 60° C. for 1 h. The mixture was quenched with sodium carbonate (10%, 100 mL) and extracted with ethyl acetate (3×30.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 1-chloro-3-iodo-2-methyl-5-nitrobenzene.

Step 2: To a solution of 1-chloro-3-iodo-2-methyl-5-nitrobenzene (760 mg, 2.55 mmol, 1.00 eq) in toluene (15.0 mL) were added diisopropylethylamine (1.34 mL, 7.66 mmol, 3.00 eq), [1,1′-bis(diphenylphosphino)ferrocene]dichloropalladium(II) (187 mg, 256 μmol, 0.10 eq) and potassium vinyltrifluoroborate (685 mg, 5.11 mmol, 2.00 eq) in portions under nitrogen. The reaction was stirred at 110° C. for 12 h. The mixture was concentrated under reduced pressure to give a residue, which was purified by silica gel column chromatography (petroleum ether/ethyl acetate=1/0 to 10/1) to afford 1-chloro-2-methyl-5-nitro-3-vinylbenzene.

Step 3: To a solution of 1-chloro-2-methyl-5-nitro-3-vinylbenzene (370 mg, 1.87 mmol, 1.00 eq) in tetrahydrofuran (6.00 mL) and ethyl acetate (6.00 mL) were added zinc chloride (12.7 μL, 271 μmol, 0.140 eq) and palladium on activated carbon (10%) (wetted with ca. 55% water) (50.0 mg) in portions under H₂ (15 Psi). The reaction was stirred at 25° C. for 1 h. The mixture was filtered, and the filtrate was concentrated under reduced pressure to give a residue. The residue was purified by silica gel column chromatography (petroleum ether/ethyl acetate=20/1 to 5/1) to afford 3-chloro-5-ethyl-4-methylaniline.

Step 4: To a solution of 3-chloro-5-ethyl-4-methylaniline (125 mg, 737 μmol, 1.00 eq) in acetonitrile (8.00 mL) were added pyridine (0.30 mL, 3.72 mmol, 5.04 eq) and phenyl chloroformate (0.11 mL, 886 μmol, 1.20 eq) in portions at 0° C. The reaction was stirred at 25° C. for 1 h. The mixture was concentrated and the obtained residue was purified by standard methods to afford phenyl (3-chloro-5-ethyl-4-methylphenyl)carbamate.

Compound 246: General procedure B with variant i) was used for the preparation from compound VIII-B employing phenyl (3-chloro-5-((1-methylpyrrolidin-3-yl)oxy)phenyl)carbamate. ¹H NMR (400 MHz, DMSO-d₆) δ=10.99 (s, 1H), 9.99 (s, 1H), 8.15 (s, 1H), 7.79 (s, 1H), 7.70-7.62 (m, 2H), 7.15 (s, 1H), 6.99 (s, 1H), 6.60 (s, 1H), 5.28 (s, 2H), 5.12 (dd, J=5.0, 13.2 Hz, 1H), 4.82 (br d, J=5.4 Hz, 1H), 4.50-4.44 (m, 1H), 4.37-4.31 (m, 1H), 2.97-2.88 (m, 2H), 2.77 (br d, J=5.5 Hz, 1H), 2.72 (br d, J=6.1 Hz, 2H), 2.62 (br d, J=1.5 Hz, 1H), 2.58 (br s, 2H), 2.29 (s, 3H), 2.04-1.98 (m, 1H), 1.80-1.71 (m, 1H). MS (ESI) m/z 527.2 [M+H]⁺

Step 1: To a solution of tert-butyl 3-(3-chloro-5-nitro-phenoxy)pyrrolidine-1-carboxylate (2.00 g, 5.83 mmol, 1.00 eq) in ethyl acetate (10.0 mL) was added hydrochloric acid in ethyl acetate (4 M, 20.0 mL, 13.7 eq). The reaction was stirred at 25° C. for 1 h. The mixture was concentrated under reduced pressure to afford 3-(3-chloro-5-nitrophenoxy)pyrrolidine.

Step 2: To a solution of 3-(3-chloro-5-nitrophenoxy)pyrrolidine (1.00 g, 4.12 mmol, 1.00 eq) in methanol (6.00 mL) was added paraformaldehyde 37% purity (6.00 mL, 80.6 mmol, 19.60 eq), acetic acid (0.23 mL, 4.11 mmol, 1.00 eq) and sodium cyanoborohydride (1.29 g, 20.6 mmol, 5.00 eq) in portions. The reaction was stirred at 25° C. for 1 h. The mixture was concentrated under reduced pressure to give a residue. The residue was purified first by silica gel column chromatography (petroleum ether/ethyl acetate to ethyl acetate/methanol=3/1 to 0/1), then by reversed phase preparative HPLC, to afford 3-(3-chloro-5-nitrophenoxy)-1-methylpyrrolidine.

Step 3: To a solution of 3-(3-chloro-5-nitrophenoxy)-1-methylpyrrolidine (460 mg, 1.79 mmol, 1.00 eq) in methanol (15.0 mL) and water (8.00 mL) was added iron powder (300 mg, 5.37 mmol, 3.00 eq) and ammonium chloride (479 mg, 8.95 mmol, 5.00 eq) in portions. The reaction was stirred at 80° C. for 1 h. The mixture was concentrated under reduced pressure to give a residue, which was diluted with water (50.0 mL) and extracted with ethyl acetate (3×20.0 mL). The combined organic layers were washed with water, dried over anhydrous sodium sulfate, filtered, and concentrated under reduced pressure to afford 3-chloro-5-((1-methylpyrrolidin-3-yl)oxy)aniline.

Step 4: To a solution of 3-chloro-5-((1-methylpyrrolidin-3-yl)oxy)aniline (290 mg, 1.28 mmol, 1.00 eq) in acetonitrile (5.00 mL) was added pyridine (0.52 mL, 6.39 mmol, 5.00 eq) and phenyl chloroformate (0.19 mL, 1.54 mmol, 1.20 eq) in portions. The reaction was stirred at 25° C. for 1 h. The mixture was concentrated and the obtained residue was purified by standard methods to afford phenyl (3-chloro-5-((1-methylpyrrolidin-3-yl)oxy) phenyl) carbamate.

Compound 247: General procedure B with variant i) was used for the preparation from compound VIII-B employing (4-(tert-butyl)phenyl)carbamate. ¹H NMR (400 MHz, DMSO-d₆) δ=10.99 (s, 1H), 9.70 (br s, 1H), 7.79 (s, 1H), 7.72-7.60 (m, 2H), 7.38 (br d, J=8.7 Hz, 2H), 7.32-7.25 (m, 2H), 5.26 (s, 2H), 5.16-5.08 (m, 1H), 4.52-4.43 (m, 1H), 4.39-4.28 (m, 1H), 2.98-2.85 (m, 1H), 2.64-2.63 (m, 1H), 2.65-2.56 (m, 1H), 2.47-2.33 (m, 1H), 2.07-1.97 (m, 1H), 1.24 (s, 9H). MS (ESI) m/z 450.2 [M+H]⁺

To a solution of phenyl carbamate (0.63 mL, 5.03 mmol, 1.50 eq) and pyridine (0.81 mL, 10.1 mmol, 3.00 eq) in acetonitrile (5.00 mL) was added 4-(tert-butyl)aniline (0.53 mL, 3.35 mmol, 1.00 eq). The reaction was stirred at 25° C. for 12 h. The mixture was diluted with ethyl acetate (50.0 mL) and water (50.0 mL). The organic layer was separated, and the aqueous phase was extracted with ethyl acetate (3×50.0 mL). The combined organic layers were dried over sodium sulfate, filtered, and concentrated and the obtained residue was purified by standard methods to afford phenyl (4-(tert-butyl)phenyl)carbamate.

Compound 248: General procedure B with variant i) was used for the preparation from compound VIII-B employing phenyl (3-chloro-4-methyl-5-(2-morpholinoethoxy)phenyl) carbamate. ¹H NMR (400 MHz, DMSO-d₆) δ=11.00 (s, 1H), 9.89 (s, 1H), 8.20 (s, 1H), 7.79 (s, 1H), 7.71-7.60 (m, 2H), 7.14 (br d, J=19.5 Hz, 2H), 5.27 (s, 2H), 5.13 (dd, J=5.1, 13.3 Hz, 1H), 4.51-4.43 (m, 1H), 4.38-4.30 (m, 1H), 4.03 (t, J=5.7 Hz, 2H), 3.58-3.55 (m, 4H), 2.96-2.87 (m, 1H), 2.72 (t, J=5.6 Hz, 2H), 2.60 (br d, J=17.5 Hz, 1H), 2.47 (br d, J=4.6 Hz, 4H), 2.35 (br d, J=4.5 Hz, 1H), 2.12 (s, 3H), 2.05-1.97 (m, 1H). MS (ESI) m/z 571.2 [M+H]⁺

Step 1: A solution of 2-chloro-1-methyl-4-nitrobenzene (12.1 mL, 58.3 mmol, 1.00 eq) and N-iodosuccinimide (14.4 g, 64.1 mmol, 1.10 eq) in sulfuric acid (100 mL) was stirred at 60° C. for 2 h. The reaction was quenched by addition of ice water (200 mL) at 0° C. The aqueous layer was extracted with ethyl acetate (3×50.0 mL). The combined organic layers were washed with brine (100 mL), dried over sodium sulfate, filtered, and concentrated under reduced pressure to give a residue. The residue was purified by silica gel column chromatography (petroleum ether/ethyl acetate=1/0) to afford 1-chloro-3-iodo-2-methyl-5-nitrobenzene.

Step 2: A solution of 1-chloro-3-iodo-2-methyl-5-nitrobenzene (7.80 g, 26.2 mmol, 1.00 eq), potassium hydroxide (4.41 g, 78.7 mmol, 3.00 eq), tris(dibenzylidenethyl acetatecetone)dipalladium(0) (1.20 g, 1.31 mmol, 0.05 eq) and 2-di-tert-butylphosphino-2′,4′,6′-triisopropylbiphenyl (557 mg, 1.31 mmol, 0.050 eq) in dioxane (80.0 mL) and water (16.0 mL) was stirred at 80° C. for 12 h under nitrogen atmosphere. The mixture was acidified to pH˜ 4 and concentrated under reduced pressure to give a residue. Brine (200 mL) was added, and the aqueous layer was extracted with ethyl acetate (3×50.0 mL). The combined organic layers were dried over sodium sulfate, filtered, and concentrated under reduced pressure to give a residue. The residue was purified by silica gel column chromatography (petroleum ether/ethyl acetate=1/0 to 17/3) to afford 3-chloro-2-methyl-5-nitrophenol.

Step 3: A suspension of 3-chloro-2-methyl-5-nitrophenol (500 mg, 2.67 mmol, 1.00 eq), 4-(2-chloroethyl) morpholine (479 mg, 3.20 mmol, 1.20 eq), potassium carbonate (553 mg, 4.00 mmol, 1.50 eq) and potassium iodide (133 mg, 0.80 mmol, 0.30 eq) in dimethylformamide (5.00 mL) was stirred at 80° C. for 1 h. The mixture was poured into brine (50.0 mL) and extracted with ethyl acetate (3×15.0 mL). The combined organic layers were dried over sodium sulfate, filtered, and concentrated under reduced pressure to afford 4-(2-(3-chloro-2-methyl-5-nitrophenoxy)ethyl)morpholine.

Step 4: To a solution of 4-(2-(3-chloro-2-methyl-5-nitrophenoxy)ethyl)morpholine (570 mg, 1.90 mmol, 1.00 eq) in methanol (5.00 mL) and water (1.00 mL) were added iron powder (529 mg, 9.48 mmol, 5.00 eq) and ammonium chloride (507 mg, 9.48 mmol, 5.00 eq). The reaction was stirred at 80° C. for 12 h under nitrogen. The mixture was filtered, and the filtrate was concentrated under reduced pressure to give a residue. The residue was treated with brine (5.00 mL) and saturated aqueous sodium bicarbonate (5.00 mL). The aqueous layer was extracted with ethyl acetate (3×10.0 mL), dried over sodium sulfate, filtered, and concentrated under reduced pressure to afford 3-chloro-4-methyl-5-(2-morpholinoethoxy)aniline.

Step 5: To a solution of 3-chloro-4-methyl-5-(2-morpholinoethoxy)aniline (300 mg, 1.11 mmol, 1.00 eq) in acetonitrile (10.0 mL) were added pyridine (0.45 mL, 5.54 mmol, 5.00 eq) and phenyl chloroformate (0.17 mL, 1.33 mmol, 1.20 eq) at 0° C. The reaction was stirred at 25° C. for 1 h. The mixture was concentrated and the obtained residue was purified by standard methods to afford phenyl (3-chloro-4-methyl-5-(2-morpholinoethoxy)phenyl)carbamate.

Compound 249: General procedure B with variant i) was used for the preparation from compound VIII-B employing phenyl (6-ethyl-5-methylpyridin-3-yl)carbamate. ¹H NMR (400 MHz, DMSO-d₆) δ=11.11-10.94 (m, 1H), 10.81-10.66 (m, 1H), 8.67 (d, J=1.6 Hz, 1H), 8.20 (br s, 1H), 7.82 (s, 1H), 7.74-7.59 (m, 2H), 5.35 (s, 2H), 5.19-5.02 (m, 1H), 4.53-4.44 (m, 1H), 4.40-4.27 (m, 1H), 3.04-2.90 (m, 3H), 2.61 (br d, J=16.5 Hz, 1H), 2.43 (s, 3H), 2.42-2.35 (m, 1H), 2.07-1.97 (m, 1H), 1.24 (t, J=7.6 Hz, 3H). MS (ESI) m/z 437.1 [M+H]⁺

Step 1: To a solution of 2-chloro-3-methyl-5-nitropyridine (2.00 g, 11.6 mmol, 1.00 eq), ethylboronic acid (2.14 g, 29.0 mmol, 2.50 eq) and potassium carbonate (4.81 g, 34.8 mmol, 3.00 eq) in dioxane (20.0 mL) was added tetrakis[triphenylphosphine]palladium(0) (1.34 g, 1.16 mmol, 0.100 eq) at 20° C. The reaction was stirred at 110° C. under nitrogen for 12 h. The mixture was concentrated under reduced pressure to give a residue. The residue was purified by silica gel column chromatography (petroleum ether/ethyl acetate=1/0 to 20/1) to afford 2-ethyl-3-methyl-5-nitropyridine.

Step 2: A mixture of 2-ethyl-3-methyl-5-nitropyridine (800 mg, 4.81 mmol, 1.00 eq), iron powder (806 mg, 14.4 mmol, 3.00 eq) and ammonium chloride (1.29 g, 24.0 mmol, 5.00 eq) in methanol (8.00 mL) and water (4.00 mL) was stirred at 80° C. for 1 h. The mixture was filtered, and the filtrate was concentrated under reduced pressure to give a residue. The residue was added to water (100 mL) and the solution was stirred for 10 min, then extracted with ethyl acetate (3×50.0 mL). The combined organic layers were washed with brine (100 mL), dried over anhydrous sodium sulfate, filtered, and concentrated under reduced pressure to afford 6-ethyl-5-methylpyridin-3-amine.

Step 3: To a solution of 6-ethyl-5-methyl-pyridin-3-amine (300 mg, 2.20 mmol, 1 eq) and pyridine (0.89 mL, 11.0 mmol, 5.00 eq) in acetonitrile (5.00 mL) was added phenyl chloroformate (0.41 mL, 3.30 mmol, 1.50 eq). The reaction was stirred at 25° C. for 1 h. The mixture was concentrated under reduced pressure to give a residue. The residue was added to water (80.0 mL) and stirred for 10 min, then extracted with ethyl acetate (3×40.0 mL). The combined organic layers were washed with brine (80.0 mL), dried over anhydrous sodium sulfate, filtered, and concentrated and the obtained residue was purified by standard methods to afford phenyl (6-ethyl-5-methylpyridin-3-yl)carbamate.

Compound 250: General procedure B with variant i) was used for the preparation from compound VIII-B employing phenyl (4-(pyridin-2-yl)phenyl)carbamate. ¹H NMR (400 MHz, DMSO-d₆) δ=11.00 (s, 1H), 10.01 (s, 1H), 8.62 (d, J=4.1 Hz, 1H), 8.29 (s, 1H), 8.03 (d, J=8.8 Hz, 2H), 7.93-7.88 (m, 1H), 7.87-7.79 (m, 2H), 7.73-7.69 (m, 1H), 7.67-7.63 (m, 1H), 7.60 (d, J=8.6 Hz, 2H), 7.29 (dd, J=5.3, 6.7 Hz, 1H), 5.30 (s, 2H), 5.13 (dd, J=5.0, 13.3 Hz, 1H), 4.53-4.44 (m, 1H), 4.41-4.30 (m, 1H), 2.96-2.86 (m, 1H), 2.65-2.58 (m, 1H), 2.41 (dq, J=4.4, 13.2 Hz, 1H), 2.06-1.99 (m, 1H). MS (ESI) m/z 471.2 [M+H]⁺

Step 1: To a solution of (4-nitrophenyl)boronic acid (1.41 g, 8.44 mmol, 1.00 eq) and 2-bromopyridine (1.20 mL, 12.7 mmol, 1.50 eq) in ethanol (35.0 mL) were added potassium carbonate (2.33 g, 16.9 mmol, 2.00 eq) and tetrakis[triphenylphosphine]palladium(0) (1.95 g, 1.69 mmol, 0.20 eq) in one portion. The reaction was stirred at 90° C. under nitrogen for 12 h. The mixture was diluted with water (30.0 mL) and exacted with ethyl acetate (3×30 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 2-(4-nitrophenyl)pyridine.

Step 2: To a solution of 2-(4-nitrophenyl)pyridine (600 mg, 3.00 mmol, 1.00 eq) in methanol (6.00 mL) and water (3.00 mL) were added ferrous powder (502 mg, 8.99 mmol, 3.00 eq) and ammonium chloride (802 mg, 15.0 mmol, 5.00 eq). The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over sodium sulfate, filtered, and concentrated under reduced pressure to afford 4-(2-pyridyl)aniline.

Step 3: To a solution of 4-(pyridin-2-yl)aniline (200 mg, 1.18 mmol, 1.00 eq) and pyridine (0.28 mL, 3.53 mmol, 3.00 eq) in acetonitrile (3.00 mL) was added phenyl chloroformate (0.22 mL, 1.76 mmol, 1.50 eq) dropwise at 0° C. The reaction was stirred at 25° C. for 12 h. The mixture was concentrated and the obtained residue was purified by standard methods to afford phenyl (4-(pyridin-2-yl)phenyl)carbamate.

Compound 251: General procedure B with variant i) was used for the preparation from compound VIII-B employing phenyl(5-methoxy-6-methylpyridin-3-yl)carbamate. ¹H NMR (400 MHz, DMSO-d₆) δ=10.99 (s, 1H), 10.70 (br s, 1H), 8.34 (s, 1H), 7.98 (s, 1H), 7.81 (s, 1H), 7.73-7.62 (m, 2H), 5.35 (s, 2H), 5.12 (dd, J=5.1, 13.3 Hz, 1H), 4.51-4.45 (m, 1H), 4.37-4.30 (m, 1H), 3.93 (s, 3H), 2.97-2.86 (m, 1H), 2.60 (br d, J=17.6 Hz, 1H), 2.50-2.49 (m, 3H), 2.40 (dq, J=4.4, 13.2 Hz, 1H), 2.07-1.95 (m, 1H). MS (ESI) m/z 439.1[M+H]⁺

Step 1: To a solution of 2-chloro-3-methoxy-5-nitro-pyridine (3.00 g, 15.9 mmol, 1.00 eq), methylboronic acid (1.90 g, 31.8 mmol, 2.00 eq) and potassium carbonate (6.60 g, 47.7 mmol, 3.00 eq) in dioxane (30.0 mL) was added tetrakis[triphenylphosphine]palladium(0) (1.84 g, 1.59 mmol, 0.10 eq) at 25° C. The reaction was stirred at 110° C. for 12 h under nitrogen. The mixture was concentrated under reduced pressure to give a residue. The residue was purified by silica gel column chromatography (petroleum ether/ethyl acetate=1/0 to 30/1) to afford 3-methoxy-2-methyl-5-nitropyridine.

Step 2: A mixture of 3-methoxy-2-methyl-5-nitropyridine (500 mg, 2.97 mmol, 1.00 eq), iron powder (498 mg, 8.92 mmol, 3.00 eq), and ammonium chloride (795 mg, 14.9 mmol, 5.00 eq) in methanol (4.00 mL) and water (2.00 mL) was stirred at 80° C. for 1 h. The mixture was filtered, and the filtrate was concentrated under reduced pressure to give a residue. The residue was added to water (80.0 mL) and stirred for 10 min. The solution was extracted with ethyl acetate (3×50.0 mL). The combined organic layers were washed with brine (80.0 mL), dried over anhydrous sodium sulfate, filtered, and concentrated under reduced pressure to afford 5-methoxy-6-methylpyridin-3-amine.

Step 3: To a solution of 5-methoxy-6-methylpyridin-3-amine (200 mg, 1.45 mmol, 1.00 eq), and pyridine (0.58 mL, 7.24 mmol, 5.00 eq) in acetonitrile (4.00 mL) was added phenyl chloroformate (0.22 mL, 1.74 mmol, 1.20 eq) dropwise at 0° C. The reaction was stirred at 25° C. for 12 h. The mixture was concentrated under reduced pressure to give a residue. The residue was diluted with water (50.0 mL) and extracted with ethyl acetate (3×40.0 mL). The combined organic layers were washed with brine (20.0 mL), dried over sodium sulfate, filtered, and concentrated and the obtained residue was purified by standard methods to afford phenyl (5-methoxy-6-methylpyridin-3-yl)carbamate.

### Compound 252:

Step 1: To a solution of 2-chloro-1-methyl-4-nitrobenzene (10.0 g, 58.3 mmol, 12.1 mL, 1.00 eq) in sulfuric acid (100 mL) was added N-iodosuccinimide (14.4 g, 64.1 mmol, 1.10 eq) in portions. The reaction was stirred at 60° C. for 2 h. The mixture was poured into water (300 mL) and extracted with ethyl acetate (3×80.0 mL). The combined organic layers were washed with brine (50 mL) and dried over anhydrous sodium sulfate, filtered, and concentrated under reduced pressure to afford 1-chloro-3-iodo-2-methyl-5-nitrobenzene.

Step 2: To a solution of 1-chloro-3-iodo-2-methyl-5-nitrobenzene (7.00 g, 23.5 mmol, 1.00 eq) and potassium hydroxide (3.96 g, 70.6 mmol, 3.00 eq) in dioxane (70.0 mL) and water (10.0 mL) were added tris(dibenzylideneacetone)dipalladium(0) (2.15 g, 2.35 mmol, 0.10 eq) and 2-di-tert-butylphosphino-2,4,6-triisopropylbiphenyl (999 mg, 2.35 mmol, 0.10 eq) under nitrogen. The reaction was stirred at 80° C. for 12 h. The mixture was diluted with water (300 mL) and extracted with ethyl acetate (3×80.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=1/0 to 10/1) to afford 3-chloro-2-methyl-5-nitrophenol.

Step 3: To a solution of 3-chloro-2-methyl-5-nitrophenol (1.00 g, 5.33 mmol, 1.00 eq) and tert-butyl 3-(((methylsulfonyl)oxy)methyl)pyrrolidine-1-carboxylate (1.64 g, 5.86 mmol, 1.10 eq) in dimethylformamide (10.0 mL) was added potassium carbonate (2.21 g, 15.9 mmol, 3.00 eq) in portions. The reaction was stirred at 80° C. for 12 h. The mixture was filtered, and the filtrate was concentrated under reduced pressure to give a residue. The residue was purified by reversed phase preparative HPLC to afford tert-butyl 3-((3-chloro-2-methyl-5-nitrophenoxy)methyl) pyrrolidine-1-carboxylate.

Step 4: To a solution of tert-butyl 3-((3-chloro-2-methyl-5-nitrophenoxy)methyl)pyrrolidine-1-carboxylate (400 mg, 1.08 mmol, 1.00 eq) and ammonium chloride (289 mg, 5.39 mmol, 5.00 eq) in methanol (4.00 mL) and water (4.00 mL) was added iron powder (181 mg, 3.24 mmol, 3.00 eq) in portions. The reaction was stirred at 80° C. for 2 h. The mixture was filtered, and the filtrate was concentrated under reduced pressure to give a residue. The residue was diluted with water (100 mL) and extracted with ethyl acetate (3×30.0 mL). The combined organic layers were washed with brine (20.0 mL), dried over anhydrous sodium sulfate, filtered, and concentrated under reduced pressure to afford tert-butyl 3-((5-amino-3-chloro-2-methylphenoxy)methyl)pyrrolidine-1-carboxylate.

Step 5: To a solution of tert-butyl 3-((5-amino-3-chloro-2-methylphenoxy)methyl)pyrrolidine-1-carboxylate (200 mg, 587 μmol, 1.00 eq) and pyridine (0.14 mL, 1.76 mmol, 3.00 eq) in acetonitrile (5.00 mL) was added phenyl chloroformate (0.11 mL, 880 μmol, 1.50 eq) dropwise at 0° C. The reaction was stirred at 25° C. for 12 h. The mixture was concentrated under reduced pressure to give a residue. The residue was purified by reversed phase preparative HPLC to afford tert-butyl 3-((3-chloro-2-methyl-5-((phenoxycarbonyl)amino)phenoxy)methyl) pyrrolidine-1-carboxylate.

Step 6: To a solution of 3-(6-(hydroxymethyl)-1-oxoisoindolin-2-yl)piperidine-2,6-dione VIII-B (100 mg, 365 μmol, 1.00 eq) and tert-butyl 3-((3-chloro-2-methyl-5-((phenoxycarbonyl)amino)phenoxy)methyl)pyrrolidine-1-carboxylate (168 mg, 365 μmol, 1.00 eq) in dimethylformamide (1.00 mL) was added sodium hydride (60% dispersion in mineral oil) (29.2 mg, 729 μmol, 2.00 eq) in portions at 0° C. The reaction was stirred at 25° C. for 1 h. The mixture was quenched slowly with 1M hydrochloric acid (2.00 mL) and concentrated under reduced pressure to afford tert-butyl 3-((3-chloro-5-((((2-(2,6-dioxopiperidin-3-yl)-3-oxoisoindolin-5-yl)methoxy)carbonyl)amino)-2-methylphenoxy)methyl)pyrrolidine-1-carboxylate.

Step 7: A solution of tert-butyl 3-((3-chloro-5-((((2-(2,6-dioxopiperidin-3-yl)-3-oxoisoindolin-5-yl)methoxy)carbonyl)amino)-2-methylphenoxy)methyl)pyrrolidine-1-carboxylate (200 mg, 312 μmol, 1.00 eq) in hydrochloric acid/ethyl acetate (3 M, 5.00 mL) was stirred at 25° C. for 1 h. The mixture was concentrated to give a residue. The residue was purified by a standard method to afford Compound 252. ¹H NMR (400 MHz, DMSO-d₆) δ=11.00 (s, 1H), 9.95 (s, 1H), 9.24 (br d, J=3.5 Hz, 2H), 7.79 (s, 1H), 7.71-7.62 (m, 2H), 7.17 (br d, J=13.4 Hz, 2H), 5.28 (s, 2H), 5.13 (dd, J=5.1, 13.3 Hz, 1H), 4.54-4.30 (m, 2H), 4.03-3.90 (m, 2H), 3.48-3.40 (m, 1H), 3.28-3.15 (m, 2H), 3.08-2.99 (m, 1H), 2.97-2.86 (m, 1H), 2.75 (td, J=7.2, 14.4 Hz, 1H), 2.61 (br d, J=17.6 Hz, 1H), 2.41 (dq, J=4.5, 13.2 Hz, 1H), 2.14 (s, 3H), 2.13-2.06 (m, 1H), 2.06-1.97 (m, 1H), 1.85-1.73 (m, 1H). MS (ESI) m/z 541.2 [M+H]⁺

Compound 253: General procedure B with variant i) was used for the preparation from compound VIII-B employing phenyl N-(6,7-dihydro-5H-cyclopenta[b]pyridin-3-yl)carbamate. ¹H NMR (400 MHz, DMSO-d₆) δ=11.01 (s, 1H), 10.59-10.34 (m, 1H), 8.67 -8.54 (m, 1H), 8.18-8.01 (m, 1H), 7.86-7.77 (m, 1H), 7.73-7.69 (m, 1H), 7.67-7.63 (m, 1H), 5.33 (s, 2H), 5.13 (dd, J=5.2, 13.4 Hz, 1H), 4.53-4.44 (m, 1H), 4.39-4.32 (m, 1H), 3.02 (br dd, J=7.5, 18.5 Hz, 4H), 2.96-2.89 (m, 1H), 2.66-2.57 (m, 1H), 2.41 (dd, J=4.4, 13.2 Hz, 1H), 2.23-2.11 (m, 2H), 2.06-1.98 (m, 1H). MS (ESI) m/z 435.1 [M+H]⁺

Step 1: To a solution of 3-nitro-6,7-dihydro-5H-cyclopenta[b]pyridine (600 mg, 3.65 mmol, 1.00 eq) in methanol (6.00 mL) and water (3.00 mL) were added ferrous powder (612 mg, 11.0 mmol, 3.00 eq) and ammonium chloride (977 mg, 18.3 mmol, 5.00 eq). The reaction was stirred at 80° C. for 2 h. The mixture was filtered, and the filtrate was concentrated under reduced pressure to give a residue. Water (30.0 mL) was added to the residue, and the mixture was exacted with ethyl acetate (3×30.0 mL). The combined organic phases were washed with brine (2×10.0 mL), dried over sodium sulfate, filtered, and concentrated under reduced pressure to afford 6,7-dihydro-5H-cyclopenta[b]pyridin-3-amine.

Step 2: To a solution of 6,7-dihydro-5H-cyclopenta[b]pyridin-3-amine (350 mg, 2.61 mmol, 1.00 eq) and phenyl chloroformate (0.49 mL, 3.91 mmol, 1.50 eq) in methanol (3.00 mL) was added pyridine (0.63 mL, 7.83 mmol, 3.00 eq) in one portion. The reaction was stirred at 25° C. for 2 h. The mixture was diluted with water (30.0 mL) and exacted with ethyl acetate (3×30.0 mL). The combined organic phases were washed with brine (2×10.0 mL), dried over sodium sulfate, filtered, and concentrated and the obtained residue was purified by standard methods to afford phenyl (6,7-dihydro-5H-cyclopenta[b]pyridin-3-yl)carbamate.

Compound 254: General procedure B with variant i) was used for the preparation from compound VIII-B employing phenyl (7,8-dihydro-5H-pyrano[4,3-b]pyridin-3-yl)carbamate. ¹H NMR (400 MHz, DMSO-d₆) δ=10.99 (s, 1H), 10.72 (s, 1H), 8.69 (s, 1H), 8.11 (s, 1H), 7.81 (s, 1H), 7.74-7.63 (m, 2H), 5.34 (s, 2H), 5.12 (dd, J=5.2, 13.2 Hz, 1H), 4.81 (s, 2H), 4.51-4.44 (m, 1H), 4.37-4.31 (m, 1H), 3.98 (t, J=5.6 Hz, 2H), 3.05 (s, 2H), 2.97-2.85 (m, 1H), 2.60 (d, J=17.6 Hz, 1H), 2.40 (dq, J=4.4, 13.2 Hz, 1H), 2.07-1.97 (m, 1H). MS (ESI) m/z 451.1 [M+H]⁺

To a solution of 7,8-dihydro-5H-pyrano[4,3-b]pyridin-3-amine (140 mg, 0.93 mmol, 1.00 eq) in acetonitrile (3.00 mL) were added phenyl chloroformate (0.13 mL, 1.03 mmol, 1.10 eq) and pyridine (0.23 mL, 2.80 mmol, 3.00 eq). The reaction was stirred at 25° C. for 2 h. The mixture was diluted with water 10.0 mL and ethyl acetate (10.0 mL). The aqueous layer was extracted with ethyl acetate (3×10.0 mL). The combined organic layers were dried over anhydrous sodium sulfate, filtered, and concentrated and the obtained residue was purified by standard methods to afford phenyl (7,8-dihydro-5H-pyrano[4,3-b] pyridin-3-yl)carbamate.

Compound 255: General procedure B with variant i) was used for the preparation from compound VIII-B employing phenyl (5-ethyl-6-methylpyridin-3-yl)carbamate. ¹H NMR (400 MHz, DMSO-d₆) δ=11.00 (s, 1H), 9.85 (br s, 1H), 8.34 (s, 1H), 8.15 (s, 1H), 7.80 (s, 1H), 7.74-7.61 (m, 3H), 5.28 (s, 2H), 5.13 (dd, J=5.2, 13.2 Hz, 1H), 4.53-4.43 (m, 1H), 4.40-4.30 (m, 1H), 2.96-2.88 (m, 1H), 2.63-2.59 (m, 1H), 2.57 (d, J=7.8 Hz, 2H), 2.46-2.41 (m, 1H), 2.38 (s, 3H), 2.06-1.98 (m, 1H), 1.14 (t, J=7.4 Hz, 3H). MS (ESI) m/z 437.3 [M+H]⁺

Step 1: To a solution of 3-bromo-2-methyl-5-nitropyridine (900 mg, 4.15 mmol, 1.00 eq), ethylboronic acid (919 mg, 12.4 mmol, 3.00 eq) and potassium carbonate (1.72 g, 12.4 mmol, 3.00 eq) in dioxane (10.0 mL) was added tetrakis[triphenylphosphine]palladium(0) (479 mg, 415 μmol, 0.100 eq) under nitrogen. The reaction was stirred at 110° C. for 12 h under nitrogen atmosphere. The mixture was concentrated under reduced pressure to give a residue. The residue was purified by silica gel column chromatography (petroleum ether/ethyl acetate=20/1 to 5/1) to afford 3-ethyl-2-methyl-5-nitropyridine.

Step 2: A mixture of 3-ethyl-2-methyl-5-nitropyridine (600 mg, 3.61 mmol, 1.00 eq), iron powder (605 mg, 10.8 mmol, 3.00 eq) and ammonium chloride (579 mg, 10.8 mmol, 3.00 eq) in methanol (10.0 mL) and water (10.0 mL) was stirred at 80° C. for 2 h. The mixture was concentrated under reduced pressure to give a residue. The residue was added to a saturated sodium bicarbonate solution (20.0 mL) and extracted with ethyl acetate (3×20.0 mL). The combined organic layers were washed with brine (20.0 mL), dried over anhydrous sodium sulfate, filtered, and concentrated under reduced pressure to afford 5-ethyl-6-methylpyridin-3-amine.

Step 3: To a solution of 5-ethyl-6-methylpyridin-3-amine (350 mg, 2.57 mmol, 1.00 eq) and pyridine (0.62 mL, 7.71 mmol, 3.00 eq) in acetonitrile (5.00 mL) was added phenyl chloroformate (0.48 mL, 3.85 mmol, 1.50 eq) at 25° C. The reaction was stirred at 25° C. for 2 h. The mixture was concentrated and the obtained residue was purified by standard methods to afford phenyl (5-ethyl-6-methylpyridin-3-yl)carbamate.

Compound 256: General procedure B with variant i) was used for the preparation from compound VIII-B employing phenyl (2,3-dihydro-1H-inden-5-yl)carbamate. ¹H NMR (400 MHz, DMSO-d₆) δ=10.99 (s, 1H), 9.66 (br s, 1H), 7.79 (s, 1H), 7.73-7.61 (m, 2H), 7.47-7.30 (m, 1H), 7.20 (br d, J=8.0 Hz, 1H), 7.14-7.06 (m, 1H), 5.26 (s, 2H), 5.13 (dd, J=5.1, 13.3 Hz, 1H), 4.55-4.43 (m, 1H), 4.39-4.31 (m, 1H), 2.92 (ddd, J=5.4, 13.6, 17.5 Hz, 1H), 2.80 (td, J=7.2, 12.7 Hz, 4H), 2.61 (br d, J=17.6 Hz, 1H), 2.41 (dd, J=4.3, 13.1 Hz, 1H), 2.06-1.89 (m, 3H). MS (ESI) m/z 434.1 [M+H]⁺

To a solution of 2,3-dihydro-1H-inden-5-amine (300 mg, 2.25 mmol, 1.00 eq) and pyridine (0.91 mL, 11.3 mmol, 5.00 eq) in acetonitrile (6.00 mL) was added phenyl chloroformate (0.42 mL, 3.38 mmol, 1.50 eq). The reaction was stirred at 25° C. for 1 h. The mixture concentrated under reduced pressure to give a residue. The residue was added to water (100 mL) and stirred for 10 min. The aqueous solution was extracted with ethyl acetate (3×40.0 mL). The combined organic layers were washed with brine (100 mL), dried over anhydrous sodium sulfate, filtered, and concentrated and the obtained residue was purified by standard methods to afford phenyl (2,3-dihydro-TH-inden-5-yl)carbamate.

Compound 257: General procedure B with variant i) was used for the preparation from compound VIII-B employing phenyl (3-fluoro-5-(trifluoromethoxy)phenyl)carbamate. ¹H NMR (400 MHz, DMSO-d₆) δ=10.99 (br s, 1H), 10.35 (s, 1H), 7.80 (s, 1H), 7.71-7.67 (m, 1H), 7.67-7.61 (m, 1H), 7.39-7.31 (m, 2H), 6.99 (br d, J=8.8 Hz, 1H), 5.30 (s, 2H), 5.13 (dd, J=5.1, 13.3 Hz, 1H), 4.52-4.42 (m, 1H), 4.38-4.30 (m, 1H), 2.98-2.85 (m, 1H), 2.63-2.57 (m, 1H), 2.43-2.35 (m, 1H), 2.06-1.97 (m, 1H). MS (ESI) m/z 496.3 [M+H]⁺

To a solution of 3-fluoro-5-(trifluoromethoxy)aniline (200 mg, 1.03 mmol, 1.00 eq) and pyridine (0.25 mL, 3.10 mmol, 3.02 eq) in acetonitrile (10.0 mL) was added phenyl chloroformate (0.15 mL, 1.23 mmol, 1.20 eq) in portions at 0° C. The reaction was stirred at 25° C. for 1 h. The mixture was concentrated and the obtained residue was purified by standard methods to afford phenyl (3-fluoro-5-(trifluoromethoxy)phenyl)carbamate.

Compound 258: General procedure B with variant i) was used for the preparation from compound VIII-B employing phenyl (4-(1-methylcyclopropyl)phenyl)carbamate. ¹H NMR (400 MHz, DMSO-d₆) δ=11.00 (s, 1H), 9.71 (br s, 1H), 7.79 (s, 1H), 7.70-7.66 (m, 1H), 7.66-7.60 (m, 1H), 7.37 (br d, J=8.4 Hz, 2H), 7.17-7.09 (m, 2H), 5.26 (s, 2H), 5.13 (dd, J=5.1, 13.3 Hz, 1H), 4.52-4.43 (m, 1H), 4.39-4.29 (m, 1H), 2.96-2.87 (m, 1H), 2.62 (br d, J=2.0 Hz, 1H), 2.43-2.35 (m, 1H), 2.05-1.98 (m, 1H), 1.34 (s, 3H), 0.80-0.73 (m, 2H), 0.73-0.66 (m, 2H). MS (ESI) m/z 448.2 [M+H]⁺

Step 1: To freshly distilled dichloromethane (50.0 mL) was added diethylzinc (1 M in toluene, 40.6 mL, 4.00 eq). The solution was cooled to −40° C., and diiodomethane (40.6 mL, 4.00 eq) in dichloromethane (10.0 mL) was added slowly. The mixture was stirred at −40° C. for 30 min, then trifluoroacetic acid (0.15 mL, 2.03 mmol, 0.20 eq) and N,N-dimethylacetamide (1.05 mL, 10.1 mmol, 1.00 eq) in dichloromethane (10.0 mL) were added. The reaction was stirred at −15° C. for 0.5 h, then 1-bromo-4-(prop-1-en-2-yl)benzene (2.00 g, 10.1 mmol, 1.00 eq) in dichloromethane (10.0 mL) was added at 0° C. The mixture was stirred at 25° C. for 12 h. The reaction was quenched with ice-water (50.0 mL) at 0° C. The organic layer was separated and concentrated under reduced pressure to give a residue. The residue was purified by silica gel column chromatography (petroleum ether 100%) to afford 1-bromo-4-(1-methylcyclopropyl)benzene.

Step 2: To a solution of 1-bromo-4-(1-methylcyclopropyl)benzene (2.00 g, 9.47 mmol, 1.00 eq) (crude) in tert-amyl alcohol (100 mL) were added tert-butyl carbamate (2.00 g, 17.1 mmol, 1.80 eq), methanesulfonato (2-di-tbutylphosphino-2,4,6-tri-ipropyl-1,1-biphenyl)(2-amino-1,1-biphenyl-2-yl)palladium(II) (600 mg, 755 μmol, 0.0800 eq) and sodium tert-butoxide (2 M in tetrahydrofuran, 14.0 mL, 2.96 eq). The reaction was stirred at 90° C. for 3 h under nitrogen. The mixture was diluted with ethyl acetate (200 mL) and water (200 mL). The organic layer was separated and concentrated under reduced pressure to give a residue. The residue was purified by reversed phase preparative HPLC to afford tert-butyl (4-(1-methylcyclopropyl)phenyl) carbamate.

Step 3: To a solution of tert-butyl (4-(1-methylcyclopropyl)phenyl) carbamate (520 mg, 2.10 mmol, 1.00 eq) in ethyl acetate (10.0 mL) was added hydrogen chloride/ethyl acetate (4 M, 10 mL, 19.0 eq). The reaction was stirred at 25° C. for 1 h. The mixture was concentrated under reduced pressure to afford 4-(1-methylcyclopropyl)aniline.

Step 4: To a solution of 4-(1-methylcyclopropyl)aniline (200 mg, 1.09 mmol, 1.00 eq, hydrochloric acid) in acetonitrile (50.0 mL) were added pyridine (0.50 mL, 6.19 mmol, 5.69 eq) and phenyl chloroformate (187 mg, 1.20 mmol, 1.10 eq) at 0° C. The reaction was stirred at 25° C. for 1 h. The mixture was concentrated and the obtained residue was purified by standard methods to afford phenyl (4-(1-methylcyclopropyl)phenyl)carbamate.

Compound 259: General procedure B with variant i) was used for the preparation from compound VIII-B employing phenyl (4-(trifluoromethoxy)pyridin-2-yl)carbamate. ¹H NMR (400 MHz, DMSO-d₆) δ=10.99 (br s, 1H), 10.80 (s, 1H), 8.38 (d, J=5.6 Hz, 1H), 7.82 (s, 2H), 7.74-7.59 (m, 2H), 7.08 (br d, J=5.5 Hz, 1H), 5.32 (s, 2H), 5.13 (br dd, J=5.0, 13.4 Hz, 1H), 4.50-4.43 (m, 1H), 4.38-4.31 (m, 1H), 2.95-2.88 (m, 1H), 2.62 (br d, J=2.1 Hz, 1H), 2.44-2.35 (m, 1H), 2.06-1.97 (m, 1H). MS (ESI) m/z 479.1 [M+H]⁺

To a solution of 4-(trifluoromethoxy)pyridin-2-amine (300 mg, 1.68 mmol, 1.00 eq) and pyridine (0.68 mL, 8.42 mmol, 5.00 eq) in acetonitrile (3.00 mL) was added phenyl chloroformate (0.25 mL, 2.02 mmol, 1.20 eq) dropwise at 0° C. The reaction was stirred at 25° C. for 3 h. The mixture was concentrated and the obtained residue was purified by standard methods to afford phenyl (4-(trifluoromethoxy)pyridin-2-yl)carbamate.

Compound 260: General procedure B with variant i) was used for the preparation from compound VIII-B employing phenyl (6-(pyrrolidin-1-yl)pyridin-3-yl)carbamate. ¹H NMR (400 MHz, DMSO-d₆) δ=11.00 (s, 1H), 9.43 (br s, 1H), 8.16 (s, 1H), 8.09 (br s, 1H), 7.77 (s, 1H), 7.64 (q, J=7.7 Hz, 2H), 7.57 (br d, J=9.4 Hz, 1H), 6.40 (d, J=8.9 Hz, 1H), 5.23 (s, 2H), 5.12 (dd, J=5.1, 13.3 Hz, 1H), 4.50-4.42 (m, 1H), 4.37-4.30 (m, 1H), 3.33 (br d, J=6.7 Hz, 4H), 2.93-2.86 (m, 1H), 2.63-2.58 (m, 1H), 2.43-2.36 (m, 1H), 2.04-1.98 (m, 1H), 1.94-1.89 (m, 4H). MS (ESI) m/z 464.2 [M+H]⁺

Step 1: To a solution of 2-chloro-5-nitropyridine (2.00 g, 12.6 mmol, 1.00 eq) in dimethylformamide (10.0 mL) were added pyrrolidine (1.58 mL, 18.9 mmol, 1.50 eq) and potassium carbonate (5.23 g, 37.8 mmol, 3.00 eq). The reaction was stirred at 60° C. for 1 h. After cooling to room temperature, the mixture was poured into ice-water (200 mL). The resulting yellow precipitate was collected by filtration and dried under reduced pressure to afford 5-nitro-2-(pyrrolidin-1-yl)pyridine.

Step 2: To a solution of 5-nitro-2-(pyrrolidin-1-yl)pyridine (2.40 g, 12.4 mmol, 1.00 eq) in methanol (240 mL) was added palladium on carbon (100 mg, 10% weight on C). The reaction was stirred at 25° C. for 1 h under hydrogen (15 Psi). The mixture was filtered, and the filtrate was concentrated under reduced pressure to afford 6-(pyrrolidin-1-yl)pyridin-3-amine.

Step 3: To a solution of 6-(pyrrolidin-1-yl)pyridin-3-amine (1.50 g, 9.19 mmol, 1.00 eq) in acetonitrile (15.0 mL) were added pyridine (3.71 mL, 45.9 mmol, 5.00 eq) and phenyl chloroformate (1.50 mL, 11.9 mmol, 1.30 eq). The reaction was stirred at 25° C. for 1 h. The mixture was concentrated and the obtained residue was purified by standard methods to afford phenyl (6-(pyrrolidin-1-yl)pyridin-3-yl)carbamate.

Compound 261: General procedure B with variant i) was used for the preparation from compound VIII-B employing phenyl (4-fluoro-3-(trifluoromethoxy)phenyl)carbamate. ¹H NMR (400 MHz, DMSO-d₆) δ=11.00 (br s, 1H), 10.16 (br s, 1H), 7.84-7.62 (m, 4H), 7.50-7.38 (m, 2H), 5.30 (br s, 2H), 5.14 (br dd, J=4.6, 13.2 Hz, 1H), 4.54-4.30 (m, 2H), 3.06-2.84 (m, 1H), 2.612 (br d, J=16.8 Hz, 1H), 2.46-2.38 (m, 1H), 2.08-1.96 (m, 1H). MS (ESI) m/z 496.1 [M+H]⁺

To a solution of 4-fluoro-3-(trifluoromethoxy)aniline (400 mg, 2.05 mmol, 1.00 eq) and phenyl chloroformate (0.28 mL, 2.26 mmol, 1.10 eq) in acetonitrile (3.00 mL) was added pyridine (0.50 mL, 6.15 mmol, 3.00 eq) in one portion. The reaction was stirred at 25° C. for 2 h. The mixture was diluted with water (30.0 mL) and exacted with ethyl acetate (3×30.0 mL). The combined organic phases were washed with brine (2×10.0 mL), dried over sodium sulfate, filtered, and concentrated and the obtained residue was purified by standard methods to afford phenyl (4-fluoro-3-(trifluoromethoxy)phenyl)carbamate.

Compound 262: General procedure B with variant i) was used for the preparation from compound VIII-B employing phenyl (4-(3-methyloxetan-3-yl)phenyl)carbamate. ¹H NMR (400 MHz, DMSO-d₆) δ=11.03 (s, 1H), 9.82 (br s, 1H), 7.83 (s, 1H), 7.74-7.65 (m, 2H), 7.48 (br d, J=8.5 Hz, 2H), 7.21 (d, J=8.6 Hz, 2H), 5.30 (s, 2H), 5.16 (dd, J=5.2, 13.3 Hz, 1H), 4.80 (d, J=5.5 Hz, 2H), 4.55-4.48 (m, 3H), 4.40 (s, 1H), 2.95 (s, 1H), 2.66 (br s, 1H), 2.44 (br dd, J=4.5, 12.9 Hz, 1H), 2.08-2.01 (m, 1H), 1.63 (s, 3H). MS (ESI) m/z 464.2 [M+H]⁺

Step 1: To a solution of diethyl 2-methylmalonate (13.6 g, 78.0 mmol, 13.3 mL, 1.10 eq) in dimethylformamide (80.0 mL) was added sodium hydride (60% dispersion in mineral oil) (3.40 g, 85.0 mmol, 1.20 eq) slowly at 0° C. The reaction was stirred at 0° C. for 0.5 h, then 1-fluoro-4-nitrobenzene (10.0 g, 70.8 mmol, 7.52 mL, 1.00 eq) was added. The mixture was stirred at 25° C. for 3 h. Water (200 mL) was added, and the mixture was extracted with ethyl acetate (3×50.0 mL). The combined organic layers were dried over sodium sulfate, filtered, and concentrated under reduced pressure to give a residue. The residue was purified by silica gel column chromatography (petroleum ether/ethyl acetate=10/1 to 8/1) to afford diethyl 2-methyl-2-(4-nitrophenyl)malonate.

Step 2: To a solution of diethyl 2-methyl-2-(4-nitrophenyl)malonate (7.00 g, 23.7 mmol, 1.00 eq) in tetrahydrofuran (15.0 mL) was added lithium aluminium hydride (953 mg, 25.1 mmol, 1.06 eq) slowly at 0° C. under nitrogen. The reaction was stirred at 0° C. for 3 h. The mixture was then partitioned between dichloromethane and 1 M hydrochloric acid. The organic layer was separated, and the aqueous layer was extracted with dichloromethane (3×50.0 mL). The combined organic layers were concentrated under reduced pressure to give a residue. The residue was purified by silica gel column chromatography (petroleum ether/ethyl acetate=10/1 to 0/1) to afford 2-methyl-2-(4-nitrophenyl)propane-1,3-diol.

Step 3: To a solution of 2-methyl-2-(4-nitrophenyl)propane-1,3-diol (200 mg, 947 μmol, 1.00 eq) in tetrahydrofuran (10.0 mL) was added n-butyllithium (2.5 M in hexane, 0.46 mL, 1.21 eq) and tosyl chloride (271 mg, 1.42 mmol, 1.50 eq) at 0° C. The reaction was stirred at 25° C. for 1 h. After cooling to 0° C., n-butyllithium (2.5 M in hexane, 0.46 mL, 1.21 eq) was added. The reaction was stirred at 65° C. for another 2 h. The reaction was quenched with saturated ammonium chloride (10 mL) and extracted with ethyl acetate (50.0 mL). The organic layer was separated and concentrated under reduced pressure to give a residue. The residue was purified by silica gel column chromatography (petroleum ether/ethyl acetate=3/1) to afford 3-methyl-3-(4-nitrophenyl)oxetane.

Step 4: To a solution of 3-methyl-3-(4-nitrophenyl)oxetane (56.0 mg, 290 μmol, 1.00 eq) in ethyl acetate (5.00 mL) was added wet palladium on carbon (10% weight on C) (20.0 mg). The reaction was stirred at 25° C. for 2 h under hydrogen (15.0 Psi). The mixture was filtered, and the filtrate was concentrated under reduced pressure to afford 4-(3-methyloxetan-3-yl)aniline.

Step 5: To a solution of 4-(3-methyloxetan-3-yl)aniline (60.0 mg, 368 μmol, 1.00 eq) in acetonitrile (3.00 mL) was added pyridine (0.15 mL, 1.84 mmol, 5.00 eq) and phenyl chloroformate (0.07 mL, 551 μmol, 1.50 eq) at 0° C. The reaction was stirred at 0° C. for 0.5 h. The mixture was filtered, and the filtrate was concentrated and the obtained residue was purified by standard methods to afford phenyl (4-(3-methyloxetan-3-yl)phenyl)carbamate.

Compound 263: General procedure B with variant i) was used for the preparation from compound VIII-B employing phenyl (2-ethylpyridin-4-yl)carbamate. ¹H NMR (400 MHz, DMSO-d₆) δ=11.37 (br d, J=10.6 Hz, 1H), 11.00 (s, 1H), 8.54 (d, J=6.7 Hz, 1H), 7.85-7.80 (m, 2H), 7.77-7.70 (m, 2H), 7.69-7.64 (m, 1H), 5.40 (s, 2H), 5.13 (dd, J=5.1, 13.3 Hz, 1H), 4.54-4.45 (m, 1H), 4.40-4.32 (m, 1H), 2.97-2.87 (m, 3H), 2.61 (br d, J=17.1 Hz, 1H), 2.41 (br dd, J=4.5, 13.0 Hz, 1H), 2.06-1.98 (m, 1H), 1.27 (t, J=7.5 Hz, 3H). MS (ESI) m/z 423.2 [M+H]⁺

To a solution of 2-ethylpyridin-4-amine (0.500 g, 4.09 mmol, 1.00 eq) and pyridine (1.65 mL, 20.4 mmol, 5.00 eq) in dimethylformamide (50.0 mL) was added phenyl chloroformate (0.77 mL, 6.19 mmol, 1.51 eq) at 0° C. The reaction was stirred at 0° C. for 12 h. The mixture was filtered and concentrated and the obtained residue was purified by standard methods to afford phenyl (2-ethylpyridin-4-yl)carbamate.

Compound 264: General procedure B with variant i) was used for the preparation from compound VIII-B employing phenyl (2-(piperidin-1-yl)pyrimidin-5-yl)carbamate. ¹H NMR (400 MHz, DMSO-d₆) δ=11.00 (s, 1H), 9.55 (br s, 1H), 8.39 (br s, 2H), 7.78 (s, 1H), 7.70-7.61 (m, 2H), 5.26 (s, 2H), 5.17-5.07 (m, 1H), 4.53-4.28 (m, 2H), 3.71-3.64 (m, 4H), 3.00-2.85 (m, 1H), 2.66-2.57 (m, 1H), 2.44-2.34 (m, 1H), 2.08-1.97 (m, 1H), 1.67-1.57 (m, 2H), 1.55-1.42 (m, 4H). MS (ESI) m/z 479.1 [M+H]⁺

Step 1: A solution of 2-chloro-5-nitro-pyrimidine (1.00 g, 6.27 mmol, 1.00 eq), piperidine (1.24 mL, 12.5 mmol, 2.00 eq) and potassium carbonate (2.60 g, 18.8 mmol, 3.00 eq) in dimethylformamide (5.00 mL) was stirred at 25° C. for 12 h. The mixture was diluted with ethyl acetate (100 mL) and water (100 mL). The organic layer was separated, and the aqueous layer was extracted with ethyl acetate (3×200 mL). The combined organic layers were dried over sodium sulfate, filtered, and concentrated under reduced pressure to afford 5-nitro-2-(piperidin-1-yl)pyrimidine.

Step 2: A mixture of 5-nitro-2-(1-piperidyl) pyrimidine (1.31 g, 6.27 mmol, 1.00 eq) and palladium on carbon (10% weight on C) (2.00 g, 6.27 mmol, 1.00 eq) in methanol (10.0 mL) was stirred at 25° C. for 12 h under hydrogen (15 Psi). The mixture was filtered over Celite, and the filtrate was concentrated under reduced pressure to afford 2-(piperidin-1-yl)pyrimidin-5-amine.

Step 3: To a solution of 2-(piperidin-1-yl)pyrimidin-5-amine (300 mg, 1.68 mmol, 1.00 eq), and pyridine (0.41 mL, 5.05 mmol, 3.00 eq) in acetonitrile (10.0 mL) was added phenyl chloroformate (0.32 mL, 2.52 mmol, 1.50 eq). The reaction was stirred at 25° C. for 12 h. The mixture was diluted with ethyl acetate (50.0 mL) and water (50.0 mL). The organic layer was separated, and the aqueous layer was extracted with ethyl acetate (3×100 mL). The combined organic layers were dried over sodium sulfate, filtered, and concentrated and the obtained residue was purified by standard methods to afford phenyl (2-(piperidin-1-yl)pyrimidin-5-yl)carbamate.

Compound 265: General procedure B with variant i) was used for the preparation from compound VIII-B employing phenyl (6-(8-oxa-3-azabicyclo[3.2.1]octan-3-yl)pyridin-3-yl)carbamate. ¹H NMR (400 MHz, DMSO-d₆) δ=11.00 (s, 1H), 9.57 (br s, 1H), 8.28 (s, 1H), 8.17 (br s, 1H), 7.78 (s, 1H), 7.72-7.58 (m, 3H), 6.69 (d, J=9.1 Hz, 1H), 5.25 (s, 2H), 5.13 (dd, J=5.1, 13.3 Hz, 1H), 4.50-4.32 (m, 4H), 3.71 (d, J=11.6 Hz, 2H), 2.97-2.89 (m, 1H), 2.88 (d, J=2.4 Hz, 1H), 2.85 (d, J=2.4 Hz, 1H), 2.65-2.58 (m, 1H), 2.46-2.35 (m, 1H), 2.06-1.98 (m, 1H), 1.88-1.78 (m, 2H), 1.77-1.70 (m, 2H). MS (ESI) m/z 506.3 [M+H]⁺

Step 1: To a solution of 2-chloro-5-nitropyridine (1.00 g, 6.31 mmol, 1.00 eq) and 8-oxa-3-azabicyclo[3.2.1]octane hydrochloride (1.00 g, 6.68 mmol, 1.06 eq, hydrochloride) in dimethylformamide (10.0 mL) was added potassium carbonate (2.62 g, 18.9 mmol, 3.00 eq) in portions. The reaction was stirred at 25° C. for 3 h. The mixture was diluted with water (100 mL) and extracted with ethyl acetate (3×30.0 mL). The combined organic layers were washed with brine (20.0 mL), dried over anhydrous sodium sulfate, filtered, and concentrated under reduced pressure to afford 3-(5-nitro-2-pyridyl)-8-oxa-3-azabicyclo[3.2.1]octane.

Step 2: To a solution of 3-(5-nitropyridin-2-yl)-8-oxa-3-azabicyclo[3.2.1]octane (800 mg, 3.40 mmol, 1.00 eq) in methanol (10.0 mL) was added palladium on carbon (10% weight on C) (80.0 mg) in one portion under nitrogen. The reaction was stirred at 25° C. for 1 h under hydrogen (15 Psi). The mixture was filtered, and the filtrate was concentrated under reduced pressure to afford 6-(8-oxa-3-azabicyclo[3.2.1]octan-3-yl)pyridin-3-amine.

Step 3: To a solution of 6-(8-oxa-3-azabicyclo[3.2.1]octan-3-yl)pyridin-3-amine (400 mg, 1.95 mmol, 1.00 eq) and pyridine (0.79 mL, 9.74 mmol, 5.00 eq) in acetonitrile (5.00 mL) was added phenyl chloroformate (0.32 mL, 2.53 mmol, 1.30 eq) at 0° C. The reaction was stirred at 25° C. for 12 h. The mixture was concentrated and the obtained residue was purified by standard methods to afford phenyl (6-(8-oxa-3-azabicyclo[3.2.1]octan-3-yl)pyridin-3-yl)carbamate.

Compound 266: General procedure B with variant i) was used for the preparation from compound VIII-B employing phenyl (2,3-dihydrobenzofuran-5-yl)carbamate. ¹H NMR (400 MHz, DMSO-d₆) δ=11.00 (s, 1H), 9.56 (br s, 1H), 7.80 (s, 1H), 7.74-7.58 (m, 2H), 7.36 (br s, 1H), 7.14 (br d, J=8.0 Hz, 1H), 6.68 (d, J=8.6 Hz, 1H), 5.26 (s, 2H), 5.14 (dd, J=5.0, 13.4 Hz, 1H), 4.54-4.44 (m, 3H), 4.42-4.30 (m, 1H), 3.18-3.10 (m, 2H), 2.98-2.86 (m, 1H), 2.62 (br d, J=16.8 Hz, 1H), 2.48-2.32 (m, 1H), 2.10-1.98 (m, 1H). MS (ESI) m/z 436.2 [M+H]⁺

To a mixture of 2,3-dihydrobenzofuran-5-amine (400 mg, 2.96 mmol, 1.00 eq) and phenyl chloroformate (0.41 mL, 3.26 mmol, 1.10 eq) in acetonitrile (3.00 mL) was added pyridine (0.72 mL, 8.88 mmol, 3.00 eq) in one portion. The reaction was stirred at 25° C. for 2 h. The mixture was diluted with water (30.0 mL) and exacted with ethyl acetate (3×30.0 mL). The combined organic phases were separated, washed with brine (2×10.0 mL), dried over sodium sulfate, filtered, and concentrated and the obtained residue was purified by standard methods to afford phenyl (2,3-dihydrobenzofuran-5-yl)carbamate.

Compound 267: General procedure B with variant i) was used for the preparation from compound VIII-B employing phenyl chroman-6-ylcarbamate. ¹H NMR (400 MHz, DMSO-d₆) δ=11.00 (s, 1H), 9.52 (br s, 1H), 7.78 (s, 1H), 7.70-7.58 (m, 2H), 7.17 (br s, 1H), 7.10 (br d, J=8.8 Hz, 1H), 6.64 (d, J=8.8 Hz, 1H), 5.24 (s, 2H), 5.13 (dd, J=5.2, 13.2 Hz, 1H), 4.51-4.42 (m, 1H), 4.39-4.30 (m, 1H), 4.11-4.03 (m, 2H), 2.97-2.86 (m, 1H), 2.69 (t, J=6.4 Hz, 2H), 2.61 (br d, J=17.6 Hz, 1H), 2.41 (dt, J=8.8, 13.2 Hz, 1H), 2.06-1.98 (m, 1H), 1.93-1.84 (m, 2H). MS (ESI) m/z 450.3 [M+H]⁺

Step 1: A solution of chromane (1.00 g, 7.45 mmol, 1.00 eq) in nitric acid (10.0 mL) was stirred at −10° C. for 1 h. The reaction mixture was diluted with ice water (50.0 mL) and then quenched by addition of sodium hydroxide aqueous solution (1 N, 50.0 mL) at 0° C. The mixture was extracted with dichloromethane (3×20.0 mL). The combined organic layers were washed with brine (20.0 mL), dried over anhydrous sodium sulfate, filtered, and concentrated under reduced pressure to give a residue. The residue was purified by silica gel column chromatography (petroleum ether/ethyl acetate=10/1 to 5/1) to afford 6-nitrochroman.

Step 2: To a solution of 6-nitrochromane (650 mg, 3.63 mmol, 1.00 eq) in ethyl acetate (6.00 mL) was added palladium on carbon (10% weight on C) (100 mg) under nitrogen atmosphere. The reaction mixture was stirred at 25° C. for 2 h under hydrogen. The mixture was filtered and concentrated under reduced pressure to afford chroman-6-amine.

Step 3: To a solution of chroman-6-amine (120 mg, 804 μmol, 1.00 eq) and pyridine (0.20 mL, 2.41 mmol, 3.00 eq) in acetonitrile (2.00 mL) was added phenyl chloroformate (0.12 mL, 965 μmol, 1.20 eq) at 25° C. The reaction was stirred at 25° C. for 12 h. The mixture was concentrated and the obtained residue was purified by standard methods to afford phenyl chroman-6-ylcarbamate.

Compound 268: General procedure B with variant i) was used for the preparation from compound VIII-B employing phenyl (3-chloro-4-methyl-5-((1-methylpyrrolidin-3-yl)oxy)phenyl)carbamate. ¹H NMR (400 MHz, DMSO-d₆) δ=11.70-11.08 (m, 1H), 10.70 (br s, 1H), 9.70 (s, 1H), 7.80 (s, 1H), 7.70-7.66 (m, 1H), 7.66-7.62 (m, 1H), 7.26 (br s, 1H), 7.12 (s, 1H), 5.30 (s, 2H), 5.08 (br dd, J=5.4, 13.0 Hz, 2H), 4.54-4.46 (m, 1H), 4.46-4.36 (m, 1H), 4.06-3.56 (m, 3H), 3.44-3.36 (m, 2H), 2.92-2.84 (m, 4H), 2.68-2.62 (m, 1H), 2.44 (dd, J=4.6, 13.2 Hz, 1H), 2.20 (s, 4H), 2.12-2.04 (m, 1H). MS (ESI) m/z 541.3 [M+H]⁺

Step 1: A solution of tert-butyl 3-(3-chloro-2-methyl-5-nitrophenoxy)pyrrolidine-1-carboxylate (730 mg, 2.05 μmol, 1.00 eq) and hydrochloric acid/ethyl acetate (10.0 mL) in ethyl acetate (20.0 mL) was stirred at 25° C. for 4 h. The mixture was concentrated under reduced pressure to give a residue. The residue was diluted with saturated sodium bicarbonate (50.0 mL) and extracted with ethyl acetate (3×20.0 mL). The combined organic layers were washed with brine (20.0 mL), dried over anhydrous sodium sulfate, filtered, and concentrated under reduced pressure to afford 3-(3-chloro-2-methyl-5-nitrophenoxy)pyrrolidine.

Step 2: To a solution of 3-(3-chloro-2-methyl-5-nitrophenoxy)pyrrolidine (400 mg, 1.56 mmol, 1.00 eq) and formaldehyde 37% (12.0 mL, 161 mmol, 103 eq) in methanol (3.00 mL) was added acetic acid (0.09 mL, 1.56 mmol, 1.00 eq) at 25° C. The reaction was stirred at 25° C. for 0.5 h. Then sodium cyanoborohydride (979 mg, 15.6 mmol, 10.0 eq) was added, and the reaction was stirred at 25° C. for another 1.5 h. The mixture was concentrated under reduced pressure to give a residue. The residue was diluted with water (50.0 mL) and extracted with ethyl acetate (3×20.0 mL). The combined organic layers were washed with brine (20.0 mL), dried over sodium sulfate, filtered, and concentrated under reduced pressure to give a residue. The residue was purified by reversed phase preparative HPLC to afford 3-(3-chloro-2-methyl-5-nitrophenoxy)-1-methylpyrrolidine.

Step 3: To a mixture of 3-(3-chloro-2-methyl-5-nitrophenoxy)-1-methylpyrrolidine (250 mg, 923 μmol, 1.00 eq), iron powder (258 mg, 4.62 mmol, 5.00 eq) and ammonium chloride (247 g, 4.62 mmol, 5.00 eq) in methanol (5.00 mL) was added water (5.00 mL) at 25° C. The reaction was stirred at 80° C. for 2 h. The mixture was concentrated under reduced pressure to give a residue. The residue was poured into saturated sodium bicarbonate (20.0 mL) and extracted with ethyl acetate (3×20.0 mL). The combined organic layers were washed with brine (20.0 mL), dried over anhydrous sodium sulfate, filtered, and concentrated under reduced pressure to afford 3-chloro-4-methyl-5-((1-methylpyrrolidin-3-yl)oxy)aniline.

Step 4: To a solution of 3-chloro-4-methyl-5-(1-methylpyrrolidin-3-yl)oxy-aniline (116 mg, 482 μmol, 1.00 eq) and phenyl chloroformate (0.66 mL, 530 μmol, 1.10 eq) in acetonitrile (5.00 mL) was added pyridine (0.12 mL, 1.45 mmol, 3.00 eq) in one portion at 25° C. The reaction was stirred at 25° C. for 2 h. The mixture was diluted with water (30.0 mL) and exacted with ethyl acetate (3×30.0 mL). The combined organic layers were washed with brine (2×10.0 mL), dried over sodium sulfate, filtered, and concentrated and the obtained residue was purified by standard methods to afford (3-chloro-4-methyl-5-((1-methylpyrrolidin-3-yl)oxy)phenyl)carbamate.

Compound 269: General procedure B with variant i) was used for the preparation from compound VIII-B employing phenyl (6-(3-methylpyrrolidin-1-yl)pyridin-3-yl)carbamate. ¹H NMR (400 MHz, DMSO-d₆) δ=11.00 (s, 1H), 9.42 (br s, 1H), 8.15 (s, 1H), 8.10 (br s, 1H), 7.78 (s, 1H), 7.70-7.62 (m, 2H), 7.58 (br d, J=7.7 Hz, 1H), 6.38 (d, J=8.9 Hz, 1H), 5.24 (s, 2H), 5.13 (dd, J=5.1, 13.3 Hz, 1H), 4.52-4.43 (m, 1H), 4.39-4.31 (m, 1H), 3.54 (dd, J=7.3, 9.9 Hz, 1H), 3.47-3.43 (m, 1H), 3.29-3.27 (m, 1H), 2.98-2.85 (m, 2H), 2.65-2.56 (m, 1H), 2.47-2.38 (m, 1H), 2.36-2.29 (m, 1H), 2.12-1.95 (m, 2H), 1.55 (qd, J=8.3, 12.1 Hz, 1H), 1.07 (d, J=6.6 Hz, 3H). MS (ESI) m/z 478.2 [M+H]⁺

Step 1: To a solution of 2-chloro-5-nitropyridine (2.00 g, 12.6 mmol, 1.00 eq) in dimethylformamide (20.0 mL) was added 3-methylpyrrolidine hydrochloride (2.30 g, 18.9 mmol, 1.50 eq) and potassium carbonate (5.23 g, 37.8 mmol, 3.00 eq). The reaction was stirred at 60° C. for 1 h. The mixture was poured into water (500 ml) at 0° C. The resulting precipitate was collected by filtration and dried under vacuum to afford 2-(3-methylpyrrolidin-1-yl)-5-nitropyridine.

Step 2: To a solution of 2-(3-methylpyrrolidin-1-yl)-5-nitropyridine (2.60 g, 12.5 mmol, 1.00 eq) in methanol (30.0 mL) was added palladium on carbon (10% weight on C) (50.0 mg). The reaction was stirred at 25° C. for 1 h under hydrogen (15 Psi). The mixture was filtered, and the filtrate was concentrated under reduced pressure to afford 6-(3-methylpyrrolidin-1-yl)pyridin-3-amine.

Step 3: To a solution of 6-(3-methylpyrrolidin-1-yl)pyridin-3-amine (1.60 g, 9.03 mmol, 1.00 eq) and pyridine (3.64 mL, 45.1 mmol, 5.00 eq) in acetonitrile (16.0 mL) was added phenyl chloroformate (1.47 mL, 11.7 mmol, 1.30 eq). The reaction was stirred at 25° C. for 1 h. The mixture was concentrated and the residue was purified by a standard method to afford phenyl(6-(3-methylpyrrolidin-1-yl)pyridin-3-yl)carbamate.

Compound 270: General procedure B with variant i) was used for the preparation from compound VIII-B employing phenyl (3-chloro-4-methyl-5-((1-methylpyrrolidin-3-yl)methoxy)phenyl)carbamate. ¹H NMR (400 MHz, DMSO-d₆) δ=11.00 (s, 1H), 10.85 (br d, J=3.7 Hz, 1H), 9.95 (s, 1H), 7.79 (s, 1H), 7.73-7.60 (m, 2H), 7.26-7.10 (m, 2H), 5.28 (s, 2H), 5.13 (dd, J=5.1, 13.3 Hz, 1H), 4.54-4.44 (m, 1H), 4.41-4.29 (m, 1H), 4.08-3.93 (m, 2H), 3.75-3.69 (m, 1H), 3.49-3.41 (m, 1H), 3.26-3.16 (m, 1H), 3.13-3.01 (m, 1H), 2.97-2.86 (m, 2H), 2.81 (t, J=4.8 Hz, 3H), 2.61 (br d, J=17.9 Hz, 1H), 2.56-2.52 (m, 1H), 2.41 (dq, J=4.4, 13.2 Hz, 1H), 2.32-2.15 (m, 1H), 2.14 (s, 3H), 2.06-1.97 (m, 1H), 1.96-1.71 (m, 1H). MS (ESI) m/z 555.2 [M+H]⁺

Step 1: A mixture of tert-butyl 3-((3-chloro-2-methyl-5-nitrophenoxy)methyl)pyrrolidine-1-carboxylate (1.26 g, 3.40 mmol, 1.00 eq) in hydrochloric acid/ethyl acetate (4.0 M, 1.13 mL) was stirred at 25° C. for 1 h. The mixture was concentrated under reduced pressure to afford 3-((3-chloro-2-methyl-5-nitrophenoxy)methyl)pyrrolidine.

Step 2: To a solution of 3-((3-chloro-2-methyl-5-nitrophenoxy)methyl)pyrrolidine (900 mg, 3.32 mmol, 1.00 eq) in 2,2,2-trifluoroethanol (10.0 mL) was added paraformaldehyde (0.46 mL, 16.6 mmol, 5.00 eq). The reaction was stirred at 60° C. for 0.5 h. Then sodium borohydride (252 mg, 6.65 mmol, 2.00 eq) was added in portions, and the reaction was stirred at 60° C. for 1 h. The mixture was diluted with water (100 mL) and extracted with ethyl acetate (3×30.0 mL). The combined organic layers were washed with brine (20.0 mL), dried over anhydrous sodium sulfate, filtered, and concentrated under reduced pressure to afford 3-((3-chloro-2-methyl-5-nitrophenoxy)methyl)-1-methylpyrrolidine.

Step 3: To a solution of 3-((3-chloro-2-methyl-5-nitrophenoxy)methyl)-1-methylpyrrolidine (850 mg, 2.99 mmol, 1.00 eq) and ammonium chloride (798 mg, 14.9 mmol, 5.00 eq) in methanol (5.00 mL) and water (5.00 mL) was added iron powder (500 mg, 8.96 mmol, 3.00 eq) in portions. The reaction was stirred at 80° C. for 2 h. The mixture was concentrated under reduced pressure to give a residue. The residue was purified by reversed phase preparative HPLC to afford 3-chloro-4-methyl-5-((1-methylpyrrolidin-3-yl)methoxy)aniline.

Step 4: To a solution of 3-chloro-4-methyl-5-((1-methylpyrrolidin-3-yl)methoxy)aniline (300 mg, 1.18 mmol, 1.00 eq) and pyridine (0.29 mL, 3.53 mmol, 3.00 eq) in acetonitrile (5.00 mL) was added phenyl chloroformate (0.22 mL, 1.77 mmol, 1.50 eq) dropwise at 0° C. The reaction was stirred at 25° C. for 12 h. The mixture was concentrated and the obtained residue was purified by standard methods to afford phenyl (3-chloro-4-methyl-5-((1-methylpyrrolidin-3-yl) methoxy)phenyl)carbamate.

Compound 271: General procedure B with variant i) was used for the preparation from compound VIII-B employing phenyl (2-ethyl-6-methylpyridin-4-yl)carbamate. ¹H NMR (400 MHz, DMSO-d₆) δ=11.00 (br s, 1H), 10.11 (s, 1H), 8.27 (br s, 1H), 7.80 (s, 1H), 7.72-7.59 (m, 2H), 7.14 (d, J=3.5 Hz, 2H), 5.30 (s, 2H), 5.13 (dd, J=5.1, 13.3 Hz, 1H), 4.51-4.31 (m, 2H), 2.98-2.87 (m, 1H), 2.61 (q, J=7.5 Hz, 3H), 2.41 (br dd, J=4.4, 13.1 Hz, 1H), 2.35 (s, 3H), 2.07-1.96 (m, 1H), 1.17 (t, J=7.6 Hz, 3H). MS (ESI) m/z 437.1[M+H]⁺

Step 1: To a mixture of 2-chloro-6-methyl-4-nitropyridine (2.00 g, 11.6 mmol, 1.00 eq), ethylboronic acid (2.57 g, 34.8 mmol, 3.00 eq) and potassium carbonate (4.81 g, 34.8 mmol, 3.00 eq) in dioxane (20.0 mL) was added tetrakis(triphenylphosphine)palladium(0) (1.34 g, 1.16 mmol, 0.100 eq) under nitrogen atmosphere. The reaction was stirred at 110° C. for 12 h. The mixture was concentrated under reduced pressure to give a residue. The residue was purified by silica gel column chromatography (petroleum ether/ethyl acetate=10/1) to afford 2-ethyl-6-methyl-4-nitropyridine.

Step 2: To a solution of 2-ethyl-6-methyl-4-nitropyridine (650 mg, 3.91 mmol, 1.00 eq) in methanol (50.0 mL) was added palladium on carbon (10% weight on C) (10.0 mg) in one portion. The reaction was stirred at 25° C. for 2 h under hydrogen (15 Psi). The mixture was filtered, and the filtrate was concentrated under reduced pressure to afford 2-ethyl-6-methylpyridin-4-amine.

Step 3: To a solution of 2-ethyl-6-methylpyridin-4-amine (450 mg, 3.30 mmol, 1.00 eq) in dimethylformamide (5.00 mL) was added sodium hydride (60% dispersion in mineral oil) (396 mg, 9.91 mmol, 3.00 eq) in portions at 0° C. The reaction was stirred at 0° C. for 0.5 h. Then phenyl chloroformate (0.50 mL, 3.96 mmol, 1.20 eq) was added dropwise at 0° C. The reaction was stirred at 25° C. for 12 h, then it was quenched with 1M hydrochloric acid and filtered. The filtrate was concentrated and the obtained residue was purified by standard methods to afford phenyl (2-ethyl-6-methylpyridin-4-yl)carbamate.

Compound 272: General procedure B with variant i) was used for the preparation from compound VIII-B employing phenyl (5-fluoro-6-(piperidin-1-yl)pyridin-3-yl)carbamate. ¹H NMR (400 MHz, DMSO-d₆) δ=11.00 (s, 1H), 9.92 (br s, 1H), 8.07 (s, 1H), 7.79 (s, 1H), 7.72-7.59 (m, 3H), 5.28 (s, 2H), 5.13 (dd, J=5.1, 13.3 Hz, 1H), 4.52-4.43 (m, 1H), 4.38-4.31 (m, 1H), 3.23 (br d, J=5.5 Hz, 4H), 2.97-2.87 (m, 1H), 2.65-2.57 (m, 1H), 2.46-2.35 (m, 1H), 2.07-1.97 (m, 1H), 1.66-1.54 (m, 6H). MS (ESI) m/z 496.2 [M+H]⁺

Step 1: To a solution of 2-chloro-3-fluoro-5-nitropyridine (0.500 g, 2.83 mmol, 1.00 eq) in dimethylformamide (5.00 mL) were added piperidine (250 mg, 2.94 mmol, 1.04 eq) and potassium carbonate (800 mg, 5.79 mmol, 2.04 eq). The reaction was stirred at 25° C. for 2 h. The mixture was poured into water (100 mL). The resulting precipitate was collected by filtration and dried under vacuum to afford 3-fluoro-5-nitro-2-(piperidin-1-yl)pyridine.

Step 2: To a solution of 3-fluoro-5-nitro-2-(piperidin-1-yl)pyridine (515 mg, 2.29 mmol, 1.00 eq) in methanol (10.0 mL) was added wet palladium on carbon (10% weight on C) (50.0 mg) under hydrogen. The mixture was stirred at 25° C. for 2 h under hydrogen (15.0 Psi). The mixture was filtered, and the filtrate was concentrated under reduced pressure to afford 5-fluoro-6-(piperidin-1-yl)pyridin-3-amine.

Step 3: To a solution of 5-fluoro-6-(piperidin-1-yl)pyridin-3-amine (440 mg, 2.25 mmol, 1.00 eq) in acetonitrile (10.0 mL) were added pyridine (0.11 mL, 11.3 mmol, 5.00 eq) and phenyl chloroformate (0.34 mL, 2.70 mmol, 1.20 eq) at 0° C. The reaction was stirred at 0° C. for 1 h. The pH was adjusted to pH=5 with formic acid (1.00 mL) and the solution was filtered. The filtrate was concentrated and the obtained residue was purified by standard methods to afford phenyl (5-fluoro-6-(piperidin-1-yl)pyridin-3-yl)carbamate.

Compound 273: General procedure B with variant i) was used for the preparation from compound VIII-B employing phenyl (6-(3,3-difluoropiperidin-1-yl)pyridin-3-yl)carbamate. ¹H NMR (400 MHz, DMSO-d₆) δ=11.00 (br s, 1H), 9.61 (br s, 1H), 8.32 (br s, 1H), 8.18 (br s, 1H), 7.79 (s, 1H), 7.72-7.60 (m, 3H), 6.91 (d, J=9.1 Hz, 1H), 5.26 (s, 2H), 5.13 (dd, J=5.1, 13.3 Hz, 1H), 4.52-4.44 (m, 1H), 4.39-4.30 (m, 1H), 3.80 (t, J=12.1 Hz, 2H), 3.51-3.47 (m, 2H), 2.98-2.86 (m, 1H), 2.64-2.58 (m, 1H), 2.47-2.36 (m, 1H), 2.11-1.99 (m, 3H), 1.77-1.69 (m, 2H). MS (ESI) m/z 514.2 [M+H]⁺

Step 1: To a solution of 2-chloro-5-nitropyridine (300 mg, 1.89 mmol, 1.00 eq) in dimethylformamide (6.00 mL) were added potassium carbonate (784 mg, 5.68 mmol, 3.00 eq) and 3,3-difluoropiperidine hydrochloride (447 mg, 2.84 mmol, 1.50 eq). The reaction was stirred at 50° C. for 2 h. The mixture was poured slowly into ice-water (40.0 mL). The resulting precipitate was collected by filtration and dried under vacuum to afford 2-(3,3-difluoropiperidin-1-yl)-5-nitropyridine.

Step 2: To a solution of 2-(3,3-difluoropiperidin-1-yl)-5-nitropyridine (452 mg, 1.86 mmol, 1.00 eq) in methanol (10.0 mL) was added wet palladium on carbon (10% weight on C) (50.0 mg) under hydrogen. The reaction was stirred at 25° C. for 3 h under hydrogen (15 Psi). The mixture was filtered, and the filtrate was concentrated under reduced pressure to afford 6-(3,3-difluoropiperidin-1-yl)pyridin-3-amine.

Step 3: To a solution of 6-(3,3-difluoropiperidin-1-yl)pyridin-3-amine (380 mg, 1.78 mmol, 1.00 eq) and pyridine (0.72 mL, 8.91 mmol, 5.00 eq) in acetonitrile (10.0 mL) was added phenyl chloroformate (0.34 mL, 2.67 mmol, 1.50 eq) at 0° C. The reaction was stirred at 0° C. for 1 h. The mixture was concentrated and the obtained residue was purified by standard methods to afford phenyl (6-(3,3-difluoropiperidin-1-yl)pyridin-3-yl)carbamate.

Compound 274: General procedure B with variant i) was used for the preparation from compound VIII-B employing phenyl (6-(2-methylpyrrolidin-1-yl)pyridin-3-yl)carbamate. ¹H NMR (400 MHz, DMSO-d₆) δ=11.00 (s, 1H), 9.42 (br s, 1H), 8.14 (s, 1H), 8.10 (br s, 1H), 7.78 (s, 1H), 7.65 (q, J=7.9 Hz, 2H), 7.57 (br d, J=7.8 Hz, 1H), 6.41 (d, J=9.1 Hz, 1H), 5.24 (s, 2H), 5.13 (dd, J=5.1, 13.3 Hz, 1H), 4.53-4.43 (m, 1H), 4.39-4.29 (m, 1H), 4.11-4.00 (m, 1H), 3.43 (ddd, J=2.4, 7.4, 9.8 Hz, 1H), 3.26-3.15 (m, 1H), 2.99-2.86 (m, 1H), 2.61 (br d, J=18.5 Hz, 1H), 2.41 (br dd, J=4.5, 13.1 Hz, 1H), 2.07-1.96 (m, 3H), 1.95-1.87 (m, 1H), 1.65 (br dd, J=2.9, 4.6 Hz, 1H), 1.13 (d, J=6.1 Hz, 3H). MS (ESI) m/z 478.3 [M+H]⁺

Step 1: To a solution of 2-chloro-5-nitropyridine (400 mg, 2.52 mmol, 1.00 eq) in dimethylformamide (6.00 mL) were added potassium carbonate (1.05 g, 7.57 mmol, 3.00 eq) and 2-methylpyrrolidine hydrochloride (460 mg, 3.78 mmol, 1.50 eq). The reaction was stirred at 50° C. for 1 h. The mixture was slowly poured into ice-water (40.0 ml). The resulting precipitate was collected by filtration and dried under vacuum to afford 2-(2-methylpyrrolidin-1-yl)-5-nitropyridine.

Step 2: To a solution of 2-(2-methylpyrrolidin-1-yl)-5-nitropyridine (512 mg, 2.47 mmol, 1.00 eq) in methanol (10.0 mL) was added wet palladium on carbon (10% weight on C) (50.0 mg) under hydrogen. The reaction was stirred at 25° C. for 3 h under hydrogen (15.0 psi). The mixture was filtered, and the filtrate was concentrated under reduced pressure to afford 6-(2-methylpyrrolidin-1-yl)pyridin-3-amine.

Step 3: To a solution of 6-(2-methylpyrrolidin-1-yl)pyridin-3-amine (371 mg, 2.09 mmol, 1.00 eq) and pyridine (0.85 mL, 10.5 mmol, 5.00 eq) in acetonitrile (10.0 mL) was added phenyl chloroformate (0.39 mL, 3.14 mmol, 1.50 eq) at 0° C. The reaction was stirred at 0° C. for 1 h. The mixture was concentrated and the obtained residue was purified by standard methods to afford phenyl (6-(2-methylpyrrolidin-1-yl)pyridine-3-yl)carbamate.

Compound 275: General procedure B with variant i) was used for the preparation from compound VIII-B employing phenyl (6-(piperidin-1-yl)pyridazin-3-yl)carbamate. ¹H NMR (400 MHz, DMSO-d₆) δ=11.08 (s, 1H), 11.00 (s, 1H), 8.24-8.15 (m, 1H), 8.13-8.04 (m, 1H), 7.82 (s, 1H), 7.76-7.60 (m, 2H), 5.34 (s, 2H), 5.13 (dd, J=5.1, 13.3 Hz, 1H), 4.52-4.45 (m, 1H), 4.39-4.33 (m, 1H), 3.70 (br s, 4H), 3.01-2.84 (m, 1H), 2.61 (br d, J=17.9 Hz, 1H), 2.45-2.36 (m, 1H), 2.07-1.97 (m, 1H), 1.65 (br s, 6H). MS (ESI) m/z 479.2 [M+H]⁺

Step 1: To a solution of 6-chloropyridazin-3-amine (500 mg, 3.86 mmol, 1.00 eq) and piperidine (0.76 mL, 7.72 mmol, 2.00 eq) in dimethylformamide (3.00 mL) was added N,N-diisopropylethylamine (1.34 mL, 7.72 mmol, 2.00 eq) dropwise. The reaction was stirred at 180° C. for 2 h under microwave irradiation. The mixture was filtered, and the filtrate was concentrated under reduced pressure to give a residue. The residue was purified by reversed phase preparative HPLC to afford 6-(piperidin-1-yl)pyridazin-3-amine.

Step 2: To a solution of 6-(piperidin-1-yl)pyridazin-3-amine (180 mg, 1.01 mmol, 1.00 eq) and pyridine (0.25 mL, 3.03 mmol, 3.00 eq) in dimethylformamide (0.50 mL) and acetonitrile (3.00 mL) was added phenyl chloroformate (0.16 mL, 1.31 mmol, 1.30 eq) dropwise at 0° C. The reaction was stirred at 25° C. for 12 h. The mixture was concentrated and the obtained residue was purified by standard methods to afford phenyl (6-(piperidin-1-yl)pyridazin-3-yl)carbamate.

Compound 276: General procedure B with variant i) was used for the preparation from compound VIII-B employing phenyl (6-morpholinopyridin-3-yl)carbamate. ¹H NMR (400 MHz, DMSO-d₆) δ=10.99 (br s, 1H), 9.61 (br s, 1H), 8.20 (br s, 1H), 7.78 (s, 1H), 7.70-7.61 (m, 3H), 6.81 (d, J=9.0 Hz, 1H), 5.25 (s, 2H), 5.12 (dd, J=5.1, 13.4 Hz, 1H), 4.51-4.43 (m, 1H), 4.37-4.30 (m, 1H), 3.70-3.66 (m, 4H), 3.35 (br s, 4H), 2.97-2.86 (m, 1H), 2.63-2.57 (m, 1H), 2.40 (br dd, J=4.6, 13.2 Hz, 1H), 2.05-1.97 (m, 1H). MS (ESI) m/z 480.2 [M+H]⁺

To a solution of 6-morpholinopyridin-3-amine (300 mg, 1.67 mmol, 1.00 eq) and pyridine (0.41 mL, 5.02 mmol, 3.00 eq) in acetonitrile (8.00 mL) was added phenyl chloroformate (0.25 mL, 2.01 mmol, 1.20 eq) in portions at 0° C. The reaction was stirred at 25° C. for 1 h. The mixture was concentrated under reduced pressure to give a residue which was diluted with ethyl acetate (6.00 mL) and water (4.00 mL). The resulting precipitate was collected by filtration and dried by standard methods to afford phenyl (6-morpholinopyridin-3-yl)carbamate.

Compound 277: General procedure B with variant i) was used for the preparation from compound VIII-B employing phenyl (6-(2-methylpiperidin-1-yl)pyridin-3-yl)carbamate. ¹H NMR (400 MHz, DMSO-d₆) δ=11.07-10.89 (m, 1H), 9.50 (br s, 1H), 8.14 (br s, 1H), 7.78 (s, 1H), 7.72-7.51 (m, 3H), 6.72 (d, J=9.3 Hz, 1H), 5.25 (s, 2H), 5.13 (dd, J=5.0, 13.3 Hz, 1H), 4.59-4.51 (m, 1H), 4.51-4.43 (m, 1H), 4.40-4.29 (m, 1H), 3.97 (br d, J=13.0 Hz, 1H), 2.99-2.86 (m, 1H), 2.79 (dt, J=2.8, 12.8 Hz, 1H), 2.65-2.57 (m, 1H), 2.48-2.38 (m, 1H), 2.07-1.96 (m, 1H), 1.73-1.54 (m, 5H), 1.47-1.31 (m, 1H), 1.02 (d, J=6.7 Hz, 3H). MS (ESI) m/z 492.2 [M+H]⁺

Step 1: To a solution of 2-chloro-5-nitropyridine (2.00 g, 12.6 mmol, 1.00 eq) in dimethylformamide (10.0 mL) were added 2-methylpiperidine (2.24 mL, 18.9 mmol, 1.50 eq) and potassium carbonate (5.23 g, 37.8 mmol, 3.00 eq). The reaction was stirred at 60° C. for 1 h. The mixture was poured into water (200 mL) and extracted with ethyl acetate (2×100 mL). The combined organic layers were dried over sodium sulfate, filtered, and concentrated under reduced pressure to afford 2-(2-methylpiperidin-1-yl)-5-nitropyridine.

Step 2: To a solution of 2-(2-methylpiperidin-1-yl)-5-nitropyridine (2.70 g, 12.2 mmol, 1.00 eq) in methanol (30.0 mL) was added palladium on carbon (50.0 mg, 10% weight on C). The reaction was stirred at 25° C. for 1 h under hydrogen (15 Psi). The mixture was filtered, and the filtrate was concentrated under reduced pressure to afford 6-(2-methylpiperidin-1-yl)pyridin-3-amine.

Step 3: To a solution of 6-(2-methylpiperidin-1-yl)pyridin-3-amine (2.20 g, 11.5 mmol, 1.00 eq) and pyridine (4.64 mL, 57.5 mmol, 5.00 eq) in acetonitrile (30.0 mL) was added phenyl chloroformate (1.73 mL, 13.8 mmol, 1.20 eq). The reaction was stirred at 25° C. for 1 h. The mixture was concentrated and the obtained residue was purified by standard methods to afford phenyl (6-(2-methylpiperidin-1-yl) pyridin-3-yl)carbamate.

Compound 278: General procedure B with variant i) was used for the preparation from compound VIII-B employing phenyl (6-(4,4-difluoropiperidin-1-yl)pyridin-3-yl) carbamate. ¹H NMR (400 MHz, DMSO-d₆) δ=11.00 (s, 1H), 9.63 (br s, 1H), 8.30-8.11 (m, 1H), 7.79 (s, 1H), 7.75-7.60 (m, 3H), 6.95 (d, J=9.2 Hz, 1H), 5.26 (s, 2H), 5.13 (dd, J=5.1, 13.3 Hz, 1H), 4.52-4.43 (m, 1H), 4.40-4.30 (m, 1H), 3.67-3.58 (m, 4H), 2.99-2.85 (m, 1H), 2.65-2.57 (m, 1H), 2.46-2.36 (m, 1H), 2.05-1.91 (m, 5H). MS (ESI) m/z 514.2 [M+H]⁺

Step 1: To a solution of 2-chloro-5-nitropyridine (2.00 g, 12.6 mmol, 1.00 eq) in dimethylformamide (10.0 mL) was added 4,4-difluoropiperidine hydrochloride (2.00 g, 12.6 mmol, 1.01 eq, HCl) and potassium carbonate (4.00 g, 28.9 mmol, 2.29 eq). The reaction was stirred at 60° C. for 2 h. The mixture was poured into water (100 mL). The resulting yellow precipitate was collected by filtration and dried under vacuum to afford 2-(4,4-difluoropiperidin-1-yl)-5-nitropyridine.

Step 2: To a solution of 2-(4,4-difluoropiperidin-1-yl)-5-nitropyridine (2.70 g, 11.1 mmol, 1.00 eq) in methanol (30.0 mL) was added palladium on carbon (50.0 mg, 10% weight on C). The reaction was stirred at 25° C. for 1 h under hydrogen (15 Psi). The mixture was filtered, and the filtrate was concentrated under reduced pressure to afford 6-(4,4-difluoropiperidin-1-yl)pyridin-3-amine.

Step 3: To a solution of 6-(4,4-difluoropiperidin-1-yl)pyridin-3-amine (2.30 g, 10.7 mmol, 1.00 eq) and pyridine (4.35 mL, 53.9 mmol, 5.00 eq) in acetonitrile (30.0 mL) was added phenyl chloroformate (1.62 mL, 12.9 mmol, 1.20 eq). The reaction was stirred at 25° C. for 1 h. The mixture was concentrated under reduced pressure to give a residue. Water (30.0 mL) was added, and the mixture was exacted with ethyl acetate (3×30.0 mL). The combined organic layers were dried, filtered, and concentrated by standard methods to afford phenyl(6-(4,4-difluoropiperidin-1-yl) pyridin-3-yl)carbamate.

Compound 279: General procedure B with variant i) was used for the preparation from compound VIII-B employing (2,2-difluorobenzo[d][1,3]dioxol-5-yl)carbamate. ¹H NMR (400 MHz, DMSO-d₆) δ=10.99 (br s, 1H), 10.06 (br s, 1H), 7.80 (s, 1H), 7.71-7.67 (m, 1H), 7.67-7.62 (m, 1H), 7.58 (d, J=1.6 Hz, 1H), 7.33 (d, J=8.8 Hz, 1H), 7.18 (dd, J=2.0, 8.8 Hz, 1H), 5.29 (s, 2H), 5.13 (dd, J=5.2, 13.2 Hz, 1H), 4.54-4.43 (m, 1H), 4.41-4.29 (m, 1H), 2.98-2.86 (m, 1H), 2.64-2.57 (m, 1H), 2.46-2.35 (m, 1H), 2.06-1.97 (m, 1H). MS (ESI) m/z 474.2 [M+H]⁺

To a solution of 2,2-difluorobenzo[d][1,3]dioxol-5-amine (500 mg, 2.57 mmol, 1.00 eq) and pyridine (0.70 mL, 8.66 mmol, 3.00 eq) in acetonitrile (5.00 mL) was added phenyl chloroformate (0.43 mL, 3.47 mmol, 1.20 eq) at 25° C. The reaction was stirred at 25° C. for 2 h. The mixture was concentrated and the obtained residue was purified by standard methods to afford phenyl (2,2-difluorobenzo[d][1,3]dioxol-5-yl)carbamate.

Compound 280: General procedure B with variant i) was used for the preparation from compound VIII-B employing phenyl (6-cyclopropylpyridin-3-yl)carbamate. ¹H NMR (400 MHz, DMSO-d₆) δ=10.99 (br s, 1H), 9.87 (br s, 1H), 8.44 (d, J=2.2 Hz, 1H), 8.30 (s, 1H), 7.79 (s, 1H), 7.74 (br d, J=7.6 Hz, 1H), 7.70-7.66 (m, 1H), 7.65-7.61 (m, 1H), 7.20 (d, J=8.6 Hz, 1H), 5.27 (s, 2H), 5.18-5.05 (m, 1H), 4.51-4.43 (m, 1H), 4.38-4.30 (m, 1H), 2.98-2.84 (m, 1H), 2.65-2.56 (m, 1H), 2.47-2.34 (m, 1H), 2.08-1.95 (m, 2H), 0.92-0.85 (m, 2H), 0.85-0.80 (m, 2H). MS (ESI) m/z 435.1 [M+H]⁺

To a solution of 6-cyclopropylpyridin-3-amine (0.500 g, 3.73 mmol, 1.00 eq) and pyridine (0.90 mL, 11.2 mmol, 3.00 eq) in acetonitrile (5.00 mL) was added phenyl chloroformate (0.56 mL, 4.47 mmol, 1.20 eq). The reaction was stirred at 25° C. for 12 h. The mixture was diluted with ethyl acetate (50.0 mL) and water (50.0 mL). The organic layer was separated, and the aqueous phase was extracted with ethyl acetate (3×100 mL). The combined organic layers were dried over sodium sulfate, filtered, and concentrated and the obtained residue was purified by standard methods to afford phenyl (6-cyclopropylpyridin-3-yl)carbamate.

Compound 281: General procedure B with variant i) was used for the preparation from compound VIII-B employing phenyl(4-(oxetan-3-yl)phenyl)carbamate. ¹H NMR (400 MHz, DMSO-d₆) δ=11.00 (br s, 1H), 9.82 (br s, 1H), 7.80 (s, 1H), 7.72-7.66 (m, 1H), 7.66-7.62 (m, 1H), 7.47 (br d, J=8.4 Hz, 2H), 7.32 (d, J=8.6 Hz, 2H), 5.28 (s, 2H), 5.20-5.07 (m, 1H), 4.97-4.85 (m, 2H), 4.58 (t, J=6.4 Hz, 2H), 4.51-4.44 (m, 1H), 4.39-4.30 (m, 1H), 4.24-4.14 (m, 1H), 2.97-2.87 (m, 1H), 2.65-2.57 (m, 1H), 2.48-2.34 (m, 1H), 2.06-1.96 (m, 1H). MS (ESI) m/z 450.2 [M+H]⁺

To a solution of 4-(oxetan-3-yl)aniline (200 mg, 1.34 mmol, 1.00 eq) and pyridine (0.32 mL, 4.02 mmol, 3.00 eq) in acetonitrile (10.0 mL) was added phenyl chloroformate (0.20 mL, 1.61 mmol, 1.20 eq). The reaction was stirred at 25° C. for 12 h. The mixture was diluted with ethyl acetate (50.0 mL) and water (50.0 mL). The organic layer was separated, and the aqueous phase was extracted with ethyl acetate (3×100 mL). The combined organic layers were dried over sodium sulfate, filtered, and concentrated and the obtained residue was purified by standard methods to afford phenyl(4-(oxetan-3-yl)phenyl)carbamate.

Compound 282: General procedure B with variant i) was used for the preparation from compound VIII-B employing phenyl (6-(4-methylpiperazin-1-yl)pyridin-3-yl)carbamate. ¹H NMR (400 MHz, DMSO-d₆) δ=10.99 (s, 1H), 9.57 (br s, 1H), 8.18 (s, 2H), 7.78 (s, 1H), 7.65 (q, J=7.8 Hz, 3H), 6.81 (d, J=9.2 Hz, 1H), 5.24 (s, 2H), 5.12 (dd, J=5.1, 13.3 Hz, 1H), 4.51-4.44 (m, 1H), 4.37-4.30 (m, 1H), 3.42-3.37 (m, 4H), 2.91 (ddd, J=5.3, 13.5, 17.5 Hz, 1H), 2.60 (br dd, J=2.0, 15.6 Hz, 1H), 2.46-2.42 (m, 4H), 2.38 (br d, J=4.4 Hz, 1H), 2.24 (s, 3H), 2.05-1.98 (m, 1H). MS (ESI) m/z 493.3 [M+H]⁺

To a solution of 6-(4-methylpiperazin-1-yl)pyridin-3-amine (500 mg, 2.60 mmol, 1.00 eq) and pyridine (1.05 mL, 13.0 mmol, 5.00 eq) in acetonitrile (5.00 mL) was added phenyl chloroformate (0.39 mL, 3.12 mmol, 1.20 eq) at 0° C. The reaction was stirred at 25° C. for 12 h. The mixture was concentrated and the obtained residue was purified by standard methods to afford phenyl (6-(4-methylpiperazin-1-yl)pyridin-3-yl)carbamate.

Compound 283: General procedure B with variant i) was used for the preparation from compound VIII-B employing phenyl (3,3-dimethyl-2,3-dihydrobenzofuran-6-yl)carbamate. ¹H NMR (400 MHz, DMSO-d₆) δ=11.00 (s, 1H), 9.74 (br s, 1H), 7.80 (s, 1H), 7.74-7.60 (m, 2H), 7.08 (d, J=8.0 Hz, 1H), 6.98 (s, 1H), 6.96-6.84 (m, 1H), 5.26 (s, 2H), 5.14 (dd, J=5.0, 13.4 Hz, 1H), 4.48 (d, J=17.4 Hz, 1H), 4.36 (d, J=17.4 Hz, 1H), 4.20 (s, 2H), 3.00-2.85 (m, 1H), 2.70-2.56 (m, 1H), 2.47-2.33 (m, 1H), 2.12-1.98 (m, 1H), 1.25 (s, 6H). MS (ESI) m/z 464.2 [M+H]⁺

Step 1: To a solution of 2-bromo-5-nitrophenol (1.00 g, 4.59 mmol, 1.00 eq) and 3-bromo-2-methylprop-1-ene (0.60 mL, 5.96 mmol, 1.30 eq) in acetone (5.00 mL) was added potassium carbonate (1.27 g, 9.17 mmol, 2.00 eq) in one portion at 25° C. The reaction was stirred for 12 h at 25° C. The mixture was diluted with water (30.0 mL) and exacted with ethyl acetate (3×30.0 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 1-bromo-2-((2-methylallyl)oxy)-4-nitrobenzene.

Step 2: To a solution of 1-bromo-2-((2-methylallyl)oxy)-4-nitrobenzene (900 mg, 3.31 mmol, 1.00 eq) in dimethylformamide (5.00 mL) were added sodium acetate (678 mg, 8.27 mmol, 2.50 eq), palladium acetate (149 mg, 662 μmol, 0.20 eq), tetraethylammonium iodide (936 mg, 3.64 mmol, 1.10 eq) and sodium formate (0.18 mL, 3.31 mmol, 1.00 eq) in one portion under nitrogen. The rection was stirred for 12 h at 100° C. The mixture was diluted with water (30.0 mL) and extracted with ethyl acetate (3×30.0 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 3,3-dimethyl-6-nitro-2H-benzofuran.

Step 3: To a solution of 3,3-dimethyl-6-nitro-2,3-dihydrobenzofuran (300 mg, 1.55 mmol, 1.00 eq) in water (3.00 mL) and methanol (6.00 mL) were added ammonium chloride (415 mg, 7.76 mmol, 5.00 eq) and iron powder (434 mg, 7.76 mmol, 5.00 eq). The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over sodium sulfate, filtered, and concentrated under reduced pressure to afford 3,3-dimethyl-2,3-dihydrobenzofuran-6-amine.

Step 4: To a solution of 3,3-dimethyl-2,3-dihydrobenzofuran-6-amine (162 mg, 993 μmol, 1.00 eq) and pyridine (0.24 mL, 2.98 mmol, 3.00 eq) in acetonitrile (5.00 mL) was added phenyl chloroformate (0.14 mL, 1.09 mmol, 1.10 eq) in one portion at 25° C. The reaction was stirred at 25° C. for 12 h. The mixture was diluted with water (30.0 mL) and extracted with ethyl acetate (3×30.0 mL). The combined organic layers were washed with brine (2×10.0 mL), dried over sodium sulfate, filtered, and concentrated and the obtained residue was purified by standard methods to afford phenyl (3,3-dimethyl-2,3-dihydrobenzofuran-6-yl)carbamate.

Compound 284: General procedure B with variant i) was used for the preparation from compound VIII-B employing phenyl (6-(2-fluorophenyl)pyridin-3-yl)carbamate. ¹H NMR (400 MHz, DMSO-d₆) δ=11.02 (s, 1H), 10.23 (br s, 1H), 8.78 (d, J=2.3 Hz, 1H), 8.07-8.00 (m, 1H), 7.92 (dt, J=1.8, 7.9 Hz, 1H), 7.83 (s, 1H), 7.80-7.74 (m, 1H), 7.74-7.69 (m, 1H), 7.68-7.63 (m, 1H), 7.45 (ddt, J=1.8, 5.3, 7.6 Hz, 1H), 7.36-7.23 (m, 2H), 5.33 (s, 2H), 5.14 (dd, J=5.1, 13.4 Hz, 1H), 4.53-4.44 (m, 1H), 4.39-4.32 (m, 1H), 2.99-2.86 (m, 1H), 2.65-2.56 (m, 1H), 2.46-2.37 (m, 1H), 2.06-1.97 (m, 1H). MS (ESI) m/z 489.2 [M+H]⁺

Step 1: To a solution of 2-chloro-5-nitropyridine (2.00 g, 12.6 mmol, 1.00 eq) in dimethylformamide (20.0 mL) were added potassium carbonate (5.23 g, 37.8 mmol, 3.00 eq), (2-fluorophenyl)boronic acid (1.77 g, 12.6 mmol, 1.00 eq) and tetrakis(triphenylphosphine)palladium (728 mg, 630 μmol, 0.05 eq) under nitrogen. The reaction was stirred at 110° C. for 12 h. The mixture was diluted with water (200 mL) and extracted with ethyl acetate (2×100 mL). The combined organic layers were washed with brine (2×30.0 mL), dried over sodium sulfate, filtered, and concentrated under reduced pressure to give a residue. The residue was purified by column chromatography (petroleum ether/ethyl acetate=10/1 to 5/1) to afford 2-(2-fluorophenyl)-5-nitropyridine.

Step 2: To a solution of 2-(2-fluorophenyl)-5-nitropyridine (2.00 g, 9.17 mmol, 1.00 eq) in methanol (15.0 mL) and water (5.00 mL) were added iron powder (2.56 g, 45.8 mmol, 5.00 eq) and ammonium chloride (3.92 g, 73.3 mmol, 8.00 eq). The reaction was stirred at 80° C. for 1 h. The mixture was filtered, and the filtrate was concentrated under reduced pressure to give a residue. The residue was diluted with water (30.0 mL) and extracted with ethyl acetate (3×30.0 mL). The combined organic layers were washed with brine (2×10.0 mL), dried over sodium sulfate, filtered, and concentrated under reduced pressure to afford 6-(2-fluorophenyl)pyridin-3-amine.

Step 3: To a solution of 6-(2-fluorophenyl)pyridin-3-amine (1.70 g, 9.03 mmol, 1.00 eq) and pyridine (3.65 mL, 45.1 mmol, 5.00 eq) in acetonitrile (20.0 mL) was added phenyl chloroformate (1.60 mL, 12.8 mmol, 1.41 eq). The reaction was stirred at 25° C. for 1 h. The mixture was concentrated under reduced pressure to give a residue. The residue was triturated with water (100 mL) and the resulting solid was collected by filtration. The filter cake was washed with water (5 ml) and dried by standard methods to afford phenyl (6-(2-fluorophenyl)pyridin-3-yl)carbamate.

Compound 285: General procedure B with variant i) was used for the preparation from compound VIII-B employing phenyl (5-fluoro-6-phenylpyridin-3-yl)carbamate. ¹H NMR (400 MHz, DMSO-d₆) δ=11.08-10.94 (m, 1H), 10.57-10.27 (m, 1H), 8.59 (d, J=1.6 Hz, 1H), 8.00-7.92 (m, 1H), 7.91-7.85 (m, 2H), 7.83 (s, 1H), 7.75-7.70 (m, 1H), 7.68-7.64 (m, 1H), 7.53-7.47 (m, 2H), 7.46-7.40 (m, 1H), 5.35 (s, 2H), 5.14 (dd, J=5.2, 13.2 Hz, 1H), 4.57-4.44 (m, 1H), 4.43-4.30 (m, 1H), 2.98-2.83 (m, 1H), 2.61 (br d, J=17.2 Hz, 1H), 2.46-2.36 (m, 1H), 2.05-1.97 (m, 1H). MS (ESI) m/z 489.3[M+H]⁺

Step 1: To a solution of 2-chloro-3-fluoro-5-nitropyridine (2.00 g, 11.3 mmol, 1.00 eq), phenylboronic acid (2.76 g, 22.6 mmol, 2.00 eq) and potassium carbonate (4.70 g, 34.0 mmol, 3.00 eq) in dioxane (20.0 mL) was added tetrakis[triphenylphosphine]palladium(0) (1.31 g, 1.13 mmol, 0.10 eq) at 25° C. The reaction was stirred at 110° C. for 12 h under nitrogen. The mixture was poured into water (120 mL) and stirred for 10 min. The aqueous phase was extracted with ethyl acetate (3×60.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=50/1) to afford 3-fluoro-5-nitro-2-phenylpyridine.

Step 2: To a solution of 3-fluoro-5-nitro-2-phenylpyridine (300 mg, 1.37 mmol, 1.00 eq), in methanol (4.00 mL) and water (2.00 mL) were added ammonium chloride (367 mg, 6.87 mmol, 5.00 eq) and iron powder (230 mg, 4.12 mmol, 3.00 eq). The reaction was stirred at 80° C. for 1 h. The mixture was filtered, and the filtrate was concentrated under reduced pressure to give a residue. Water (80.0 mL) was added, and the solution was stirred for 10 min. The aqueous layer was extracted with ethyl acetate (3×30.0 mL). The combined organic layers were washed with brine (50.0 mL), dried over anhydrous sodium sulfate, filtered, and concentrated under reduced pressure to afford 5-fluoro-6-phenylpyridin-3-amine.

Step 3: To a solution of 5-fluoro-6-phenylpyridin-3-amine (250 mg, 1.33 mmol, 1.00 eq) and pyridine (0.54 mL, 6.64 mmol, 5.00 eq) in acetonitrile (5.00 mL) was added phenyl chloroformate (0.25 mL, 1.99 mmol, 1.50 eq). The reaction was stirred at 25° C. for 1 h. The mixture was concentrated and the obtained residue was purified by standard methods to afford phenyl (5-fluoro-6-phenylpyridin-3-yl)carbamate.

Compound 286: General procedure B with variant i) was used for the preparation from compound VIII-B employing phenyl (6-(o-tolyl)pyridin-3-yl)carbamate. ¹H NMR (400 MHz, DMSO-d₆) δ=11.02 (s, 1H), 10.69-10.33 (m, 1H), 8.95-8.73 (m, 1H), 8.35-8.00 (m, 1H), 7.86-7.63 (m, 4H), 7.47-7.29 (m, 4H), 5.36 (br s, 2H), 5.14 (dd, J=5.0, 13.4 Hz, 1H), 4.52-4.45 (m, 1H), 4.39-4.32 (m, 1H), 2.96-2.88 (m, 1H), 2.64-2.58 (m, 1H), 2.45-2.37 (m, 1H), 2.32 (s, 3H), 2.06-1.98 (m, 1H). MS (ESI) m/z 485.2 [M+H]⁺

Step 1: To a solution of 2-chloro-5-nitropyridine (3.00 g, 18.9 mmol, 1.00 eq) in dimethylformamide (30.0 mL) were added O-tolylboronic acid (2.57 g, 18.9 mmol, 1.00 eq), potassium carbonate (7.85 g, 56.7 mmol, 3.00 eq) and tetrakis(triphenylphosphine)palladium (1.09 g, 946 μmol, 0.05 eq) under nitrogen. The reaction was stirred at 110° C. for 12 h. The mixture was diluted with water (300 mL) and exacted with ethyl acetate (3×300 mL). The combined organic layers were washed with brine (2×100 mL), dried over sodium sulfate, filtered, and concentrated under reduced pressure to give a residue. The residue was purified by silica gel column chromatography (petroleum ether/ethyl acetate=10/1) to afford 5-nitro-2-(o-tolyl)pyridine.

Step 2: To a solution of 5-nitro-2-(o-tolyl)pyridine (3.00 g, 14.0 mmol, 1.00 eq) in methanol (30.0 mL) and water (10.0 mL) were added iron powder (3.91 g, 70.0 mmol, 5.00 eq) and ammonium chloride (5.99 g, 112 mmol, 8.00 eq). The reaction was stirred at 80° C. for 1 h. The mixture was filtered, and the filtrate was concentrated under reduced pressure to give a residue. The residue was diluted with water (50.0 mL) and exacted with ethyl acetate (3×30.0 mL). The combined organic layers were washed with brine (2×10.0 mL), dried over sodium sulfate, filtered, and concentrated under reduced pressure to afford 6-(o-tolyl)pyridin-3-amine.

Step 3: To a solution of 6-(o-tolyl)pyridin-3-amine (2.50 g, 13.5 mmol, 1.00 eq) in acetonitrile (25.0 mL) were added pyridine (5.48 mL, 67.8 mmol, 5.00 eq) and phenyl chloroformate (2.21 mL, 17.6 mmol, 1.30 eq). The reaction was stirred at 25° C. for 1 h. The mixture was concentrated under reduced pressure to give a residue. The residue was triturated with water (100 mL) and the resulting solid was collected by filtration. The filter cake was washed with water (5.00 ml) and dried under standard methods to afford phenyl (6-(o-tolyl)pyridin-3-yl)carbamate.

Compound 287: General procedure B with variant i) was used for the preparation from compound VIII-B employing phenyl (6-(2-methoxyphenyl)pyridin-3-yl)carbamate. ¹H NMR (400 MHz, DMSO-d₆) δ=11.02 (s, 1H), 10.75 (br s, 1H), 8.91 (br d, J=1.6 Hz, 1H), 8.32 (br d, J=8.7 Hz, 1H), 8.11 (d, J=8.9 Hz, 1H), 7.84 (s, 1H), 7.76-7.62 (m, 3H), 7.58-7.50 (m, 1H), 7.25 (d, J=8.3 Hz, 1H), 7.15 (t, J=7.5 Hz, 1H), 5.37 (s, 2H), 5.13 (dd, J=5.0, 13.3 Hz, 1H), 4.53-4.46 (m, 1H), 4.38-4.31 (m, 1H), 3.87 (s, 3H), 2.97-2.88 (m, 1H), 2.61 (br d, J=17.1 Hz, 1H), 2.45-2.35 (m, 1H), 2.07-1.97 (m, 1H). MS (ESI) m/z 501.2 [M+H]⁺

Step 1: To a solution of 2-chloro-5-nitropyridine (2.00 g, 12.6 mmol, 1.00 eq) in dimethylformamide (5 mL) were added potassium carbonate (5.23 g, 37.8 mmol, 3.00 eq), (2-fluorophenyl)boronic acid (1.92 g, 12.6 mmol, 1.00 eq) and tetrakis(triphenylphosphine)palladium (1.46 g, 1.26 mmol, 0.10 eq) under nitrogen. The reaction was stirred at 100° C. for 12 h. The mixture was diluted with water (300 mL) and exacted with ethyl acetate (3×300 mL). The combined organic layers were washed with brine (2×100 mL), dried over sodium sulfate, filtered, and concentrated under reduced pressure to give a residue. The residue was purified by silica gel column chromatography (petroleum ether/ethyl acetate=10/1) to afford 2-(2-methoxyphenyl)-5-nitropyridine.

Step 2: To a solution of 2-(2-methoxyphenyl)-5-nitropyridine (2.90 g, 12.6 mmol, 1.00 eq) in methanol (30.0 mL) and water (10.0 mL) were added iron powder (3.52 g, 62.9 mmol, 5.00 eq) and ammonium chloride (5.39 g, 100 mmol, 8.00 eq). The reaction was stirred at 80° C. for 1 h. The mixture was filtered, and the filtrate was concentrated under reduced pressure to give a residue. The residue was diluted with water (50.0 mL) and extracted with ethyl acetate (3×50.0 mL). The combined organic layers were washed with brine (2×10.0 mL), dried over sodium sulfate, filtered, and concentrated under reduced pressure to afford 6-(2-methoxyphenyl)pyridin-3-amine.

Step 3: To a solution of 6-(2-methoxyphenyl)pyridin-3-amine (2.50 g, 12.4 mmol, 1.00 eq) in acetonitrile (25.0 mL) were added pyridine (3.02 mL, 37.4 mmol, 3.00 eq) and phenyl chloroformate (2.03 mL, 16.2 mmol, 1.30 eq). The reaction was stirred at 25° C. for 1 h. The mixture was concentrated under reduced pressure to give a residue. The residue was triturated with water (100 mL) and the resulting solid was collected by filtration. The filter cake was washed with water (5.00 mL) and dried by standard methods to afford phenyl (6-(2-methoxyphenyl)pyridin-3-yl)carbamate.

Compound 288: General procedure B with variant i) was used for the preparation from compound VIII-B employing phenyl (5-methyl-6-phenylpyridin-3-yl)carbamate. ¹H NMR (400 MHz, DMSO-d₆) δ=11.02 (s, 1H), 10.76 (br s, 1H), 8.80-8.76 (m, 1H), 8.26 (s, 1H), 7.84 (s, 1H), 7.76-7.72 (m, 1H), 7.68-7.66 (m, 1H), 7.64-7.62 (m, 2H), 7.60 (br s, 3H), 5.38 (s, 2H), 5.22-5.04 (m, 1H), 4.50 (d, J=17.6 Hz, 1H), 4.36 (d, J=17.6 Hz, 1H), 2.98-2.86 (m, 1H), 2.68-2.58 (m, 1H), 2.46-2.34 (m, 4H), 2.10-1.98 (m, 1H). MS (ESI) m/z 485.4 [M+H]⁺

Step 1: To a solution of 2-chloro-3-methyl-5-nitropyridine (5.00 g, 29.0 mmol, 1.00 eq) and phenylboronic acid (4.24 g, 34.8 mmol, 1.20 eq) in dioxane (50.0 mL) were added tetrakis[triphenylphosphine]palladium(0) (6.70 g, 5.79 mmol, 0.20 eq) and potassium carbonate (6.01 g, 43.5 mmol, 1.50 eq) in one portion at 25° C. under nitrogen. The reaction was stirred at 100° C. for 12 h. The mixture was diluted with water (30.0 mL) and exacted with ethyl acetate (3×30.0 mL). The combined organic layers were washed with brine (2×10.0 mL), dried over sodium sulfate, filtered, and concentrated under reduced pressure to give a residue. The residue was purified by silica gel column chromatography (petroleum ether/ethyl acetate=10/1 to 2/1) to afford 3-methyl-5-nitro-2-phenyl-pyridine.

Step 2: To a solution of 3-methyl-5-nitro-2-phenylpyridine (1.00 g, 4.67 mmol, 1.00 eq) in methanol (6.00 mL) and water (3.00 mL) were added iron powder (1.30 g, 23.3 mmol, 5.00 eq) and ammonium chloride (1.25 g, 23.3 mmol, 5.00 eq) in one portion at 25° C. The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over sodium sulfate, filtered, and concentrated under reduced pressure to afford 5-methyl-6-phenylpyridin-3-amine.

Step 3: To a solution of 5-methyl-6-phenylpyridin-3-amine (843 mg, 4.58 mmol, 1.00 eq) and pyridine (1.11 mL, 13.7 mmol, 3.00 eq) in acetonitrile (2.00 mL) was added phenyl chloroformate (0.63 mL, 5.03 mmol, 1.10 eq) at 25° C. The reaction was stirred at 25° C. for 12 h. The mixture was concentrated and the obtained residue was purified by standard methods to afford phenyl (5-methyl-6-phenylpyridin-3-yl)carbamate.

Compound 289: General procedure B with variant i) was used for the preparation from compound VIII-B employing phenyl (6-(3-(dimethylamino)azetidin-1-yl)pyridin-3-yl)carbamate. ¹H NMR (400 MHz, DMSO-d₆) δ=10.99 (s, 1H), 9.52 (br s, 1H), 8.17 (s, 1H), 8.12 (br s, 1H), 7.77 (s, 1H), 7.69-7.56 (m, 3H), 6.37 (d, J=8.9 Hz, 1H), 5.24 (s, 2H), 5.12 (dd, J=5.1, 13.3 Hz, 1H), 4.51-4.43 (m, 1H), 4.38-4.30 (m, 1H), 3.92 (t, J=7.4 Hz, 2H), 3.63 (dd, J=5.6, 8.1 Hz, 2H), 3.15 (quin, J=6.1 Hz, 1H), 2.97-2.86 (m, 1H), 2.64-2.56 (m, 1H), 2.45-2.38 (m, 1H), 2.10 (s, 6H), 2.04-1.98 (m, 1H). MS (ESI) m/z 493.2 [M+H]⁺

Step 1: To a solution of 2-fluoro-5-nitropyridine (2.00 g, 14.1 mmol, 1.00 eq) and N,N-dimethylazetidin-3-amine dihydrochloride (3.65 g, 21.1 mmol, 1.50 eq, 2 HCl) in dimethylformamide (20.0 mL) was added potassium carbonate (9.73 g, 70.4 mmol, 5.00 eq) in one portion. The reaction was stirred at 60° C. for 12 h. The mixture was diluted with water (150 mL) and extracted with ethyl acetate (3×80.0 mL). The combined organic layers were washed with brine (60.0 mL), dried over sodium sulfate, filtered, and concentrated under reduced pressure to afford N,N-dimethyl-1-(5-nitropyridin-2-yl)azetidin-3-amine.

Step 2: To a solution of N,N-dimethyl-1-(5-nitropyridin-2-yl)azetidin-3-amine (2.00 g, 9.00 mmol, 1.00 eq) in methanol (40.0 mL) was added palladium on carbon (200 mg, 10% weight on C) in one portion under hydrogen (15 Psi). The reaction was stirred at 25° C. for 2 h. The mixture was filtered, and the filtrate was concentrated under reduced pressure to afford 6-(3-(dimethylamino)azetidin-1-yl)pyridin-3-amine.

Step 3: To a solution of 6-(3-(dimethylamino)azetidin-1-yl)pyridin-3-amine (500 mg, 2.60 mmol, 1.00 eq) and pyridine (1.05 mL, 13.0 mmol, 5.00 eq) in acetonitrile (5.00 mL) was added phenyl chloroformate (0.39 mL, 3.12 mmol, 1.20 eq) dropwise at 0° C. The reaction was stirred at 20° C. for 12 h. The mixture was concentrated and the obtained residue was purified by standard methods to afford phenyl (6-(3-(dimethylamino)azetidin-1-yl)pyridin-3-yl)carbamate.

Compound 290: General procedure B with variant i) was used for the preparation from compound VIII-B employing phenyl (5-methoxy-6-phenylpyridin-3-yl)carbamate. ¹H NMR (400 MHz, DMSO-d₆) δ=11.02 (s, 1H), 10.80-10.36 (m, 1H), 8.60-8.38 (m, 1H), 8.12-7.92 (m, 1H), 7.88-7.78 (m, 3H), 7.76-7.64 (m, 2H), 7.50 (br d, J=7.2 Hz, 3H), 5.36 (s, 2H), 5.14 (dd, J=5.2, 13.2 Hz, 1H), 4.50 (d, J=17.6 Hz, 1H), 4.36 (d, J=17.6 Hz, 1H), 3.90 (s, 3H), 2.99-2.86 (m, 1H), 2.62 (br d, J=17.z Hz, 1H), 2.50-2.34 (m, 1H), 2.10-1.94 (m, 1H). MS (ESI) m/z 501.2 [M+H]⁺

Step 1: To a solution of 2-chloro-3-methoxy-5-nitropyridine (1.00 g, 5.30 mmol, 1.00 eq) and phenylboronic acid (776 mg, 6.36 mmol, 1.20 eq) in dioxane (50.0 mL) were added tetrakis[triphenylphosphine]palladium(0) (1.23 g, 1.06 mmol, 0.20 eq) and potassium carbonate (1.10 g, 7.95 mmol, 1.50 eq) in one portion at 25° C. under nitrogen. The reaction was stirred at 100° C. for 12 h. The mixture was diluted with water (30.0 mL) and exacted with ethyl acetate (3×30.0 mL). The combined organic layers were washed with brine (2×10.0 mL), dried over anhydrous sodium sulfate, filtered, and concentrated under reduced pressure to give a residue. The residue was purified by silica gel column chromatography (petroleum ether/ethyl acetate=10/1 to 2/1) to afford 3-methoxy-5-nitro-2-phenylpyridine.

Step 2: To a solution of 3-methoxy-5-nitro-2-phenylpyridine (1.00 g, 4.34 mmol, 1.00 eq) in methanol (6.00 mL) and water (3.00 mL) were added iron powder (1.21 g, 21.7 mmol, 5.00 eq) and ammonium chloride (1.16 g, 21.7 mmol, 5.00 eq) in one portion at 25° C. The reaction was stirred at 80° C. for 2 h. The mixture was filtered, and the filtrate was concentrated under reduced pressure to give a residue. The residue was diluted with water (30.0 mL) and exacted with ethyl acetate (3×30.0 mL). The combined organic layers were washed with brine (2×10.0 mL), dried over anhydrous sodium sulfate, filtered, and concentrated under reduced pressure to afford 5-methoxy-6-phenylpyridin-3-amine.

Step 3: To a solution of 5-methoxy-6-phenylpyridin-3-amine (700 mg, 3.50 mmol, 1.00 eq) and pyridine (0.85 mL, 10.5 mmol, 3.00 eq) in acetonitrile (2.00 mL) was added phenyl chloroformate (0.48 mL, 3.85 mmol, 1.10 eq) at 25° C. The reaction was stirred at 25° C. for 12 h. The mixture was concentrated and the obtained residue was purified by standard methods to afford phenyl (5-methoxy-6-phenylpyridin-3-yl)carbamate.

Compound 291: General procedure B with variant i) was used for the preparation from compound VIII-B employing phenyl (6-methyl-5-(trifluoromethoxy)pyridin-3-yl)carbamate. ¹H NMR (400 MHz, DMSO-d₆) δ=11.01 (br s, 1H), 10.30 (br s, 1H), 8.50 (d, J=2.0 Hz, 1H), 7.99 (br s, 1H), 7.80 (s, 1H), 7.72-7.67 (m, 1H), 7.66-7.62 (m, 1H), 5.30 (s, 2H), 5.16-5.09 (m, 1H), 4.52-4.43 (m, 1H), 4.38-4.30 (m, 1H), 2.97-2.85 (m, 1H), 2.65-2.63 (m, 1H), 2.65-2.58 (m, 1H), 2.40 (s, 3H), 2.40-2.33 (m, 1H), 2.05-1.97 (m, 1H). MS (ESI) m/z 493.2 [M+H]⁺

Step 1: To a solution of 2-methyl-5-nitropyridin-3-amine (10.0 g, 65.3 mmol, 1.00 eq) in sulfuric acid (2.50 M, 107 mL, 4.10 eq) was added sodium nitrite (5.41 g, 78.4 mmol, 1.20 eq) dissolved in water (20.0 mL) dropwise at 0° C. The reaction was stirred at 0° C. for 0.5 h. Then sulfuric acid (1 M, 53.6 mL, 0.820 eq) was added dropwise, and the reaction was stirred at 70° C. for 1 h. The mixture was diluted with water (200 mL) and extracted with ethyl acetate (3×50.0 mL). The combined organic layers were washed brine (40.0 mL), dried over sodium sulfate, filtered, and concentrated under reduced pressure to give a residue. The residue was purified by silica gel column chromatography (petroleum ether/ethyl acetate=10/1 to 2/1) to afford 2-methyl-5-nitropyridin-3-ol.

Step 2: To a solution of 2-methyl-5-nitropyridin-3-ol (800 mg, 5.19 mmol, 1.00 eq) in dimethylformamide (8.00 mL) was added sodium hydride (60% dispersion in mineral oil) (415 mg, 10.4 mmol, 2.00 eq) in portions at 0° C. The reaction was stirred at 0° C. for 0.5 h. Then dibromodifluoromethane (0.96 mL, 10.4 mmol, 2.00 eq) was added dropwise at 0° C., and the reaction was stirred at 25° C. for 2 h. The mixture was quenched with an ammonium chloride solution (100 mL) and extracted with ethyl acetate (3×50.0 mL). The combined organic layers were washed with brine (40.0 mL), dried over sodium sulfate, filtered, and concentrated under reduced pressure to give a residue. The residue was purified by silica gel column chromatography (petroleum ether/ethyl acetate=10/1 to 5/1) to afford 3-(bromodifluoromethoxy)-2-methyl-5-nitropyridine.

Step 3: To a solution of 3-(bromodifluoromethoxy)-2-methyl-5-nitropyridine (180 mg, 636 μmol, 1.00 eq) in dichloromethane (3.00 mL) was added silver tetrafluoroborate (186 mg, 954 μmol, 1.50 eq) in portions. The reaction was stirred at 25° C. for 1 h. The mixture was filtered, and the filtrate was concentrated under reduced pressure to afford 2-methyl-5-nitro-3-(trifluoromethoxy)pyridine.

Step 4: A mixture of 2-methyl-5-nitro-3-(trifluoromethoxy)pyridine (100 mg, 450 μmol, 1.00 eq) and palladium on carbon (10% weight on C) (10.0 mg) in methanol (300 mL) was stirred at 25° C. for 2 h under hydrogen atmosphere (15 psi). The mixture was filtered over Celite, and the filtrate was concentrated under reduced pressure to afford 6-methyl-5-(trifluoromethoxy)pyridin-3-amine.

Step 5: A solution of phenyl chloroformate (16.0 μL, 124 μmol, 1.20 eq), 6-methyl-5-(trifluoromethoxy)pyridin-3-amine (20.0 mg, 104 μmol, 1.00 eq) and pyridine (25.0 μL, 312 μmol, 3.00 eq) in acetonitrile (10.0 mL) was stirred at 25° C. for 12 h. The mixture was concentrated and the obtained residue was purified by standard methods to afford phenyl (6-methyl-5-(trifluoromethoxy) pyridin-3-yl)carbamate.

Compound 292: General procedure B with variant i) was used for the preparation from compound VIII-B employing phenyl chroman-7-ylcarbamate. ¹H NMR (400 MHz, DMSO-d₆) δ=11.00 (s, 1H), 9.66 (br s, 1H), 7.79 (s, 1H), 7.72-7.61 (m, 2H), 7.00-6.84 (m, 3H), 5.25 (s, 2H), 5.13 (dd, J=5.2, 13.2 Hz, 1H), 4.53-4.42 (m, 1H), 4.40-4.29 (m, 1H), 4.16-4.02 (m, 2H), 2.99-2.85 (m, 1H), 2.68-2.58 (m, 3H), 2.41 (dq, J=4.4, 13.2 Hz, 1H), 2.07-1.97 (m, 1H), 1.95-1.83 (m, 2H). MS (ESI) m/z 450.2 [M+H]⁺

Step 1: To a mixture of chroman-6-amine (1.20 g, 8.04 mmol, 1.00 eq) in dioxane (2.00 mL) was added acetic anhydride (1.51 mL, 16.1 mmol, 2.00 eq) and pyridine (0.65 mL, 8.04 mmol, 1.00 eq) at 0° C. The reaction was stirred at 25° C. for 16 h. The mixture was poured into water (20.0 mL) and extracted with ethyl acetate (3×20.0 mL). The combined organic layers were separated, washed with brine (10.0 mL), dried over anhydrous sodium sulfate, filtered, and concentrated under reduced pressure to afford N-(chroman-6-yl)acetamide.

Step 2: A solution of nitric acid (0.46 mL, 10.3 mmol, 1.40 eq) in acetic acid (2.00 mL) was added dropwise to a stirred solution of N-(chroman-6-yl)acetamide (1.40 g, 7.32 mmol, 1.00 eq) in acetic acid (10.0 mL) at 25° C. The reaction was stirred at 25° C. for 1 h. Then ice water (50.0 mL) was added, and the reaction was stirred at 25° C. for 0.5 h. The mixture was poured into water (20.0 mL) and extracted with ethyl acetate (3×20.0 mL). The combined organic layers were washed with brine (10.0 mL), dried over anhydrous sodium sulfate, filtered, and concentrated under reduced pressure to give a residue. The residue was purified by reversed phase preparative HPLC to afford N-(7-nitrochroman-6-yl)acetamide carbamate.

Step 3: A solution of N-(7-nitrochroman-6-yl)acetamide (410 mg, 1.74 mmol, 1.00 eq) and concentrated hydrochloric acid (2.60 mL) in ethanol (10.0 mL) was stirred at 80° C. for 2 h. The reaction mixture was neutralized with ammonium hydroxide solution and diluted with water (20.0 mL), then extracted with ethyl acetate (3×20.0 mL). The combined organic layers were washed with brine (10.0 mL), dried over anhydrous sodium sulfate, filtered, and concentrated under reduced pressure to afford 7-nitrochroman-6-amine.

Step 4: To a mixture of 7-nitrochroman-6-amine (330 mg, 1.70 mmol, 1.00 eq) in tetrahydrofuran (10.0 mL) was added isoamyl nitrite (0.69 mL, 5.10 mmol, 3.00 eq) dropwise at 0° C. The reaction was stirred at 0° C. for 30 min, then at 70° C. for 3 h. The mixture was poured into water (20.0 mL) and extracted with ethyl acetate (3×20.0 mL). The combined organic layers were separated, washed with brine (10.0 mL), dried over anhydrous sodium sulfate, filtered, and concentrated under reduced pressure to give a residue. The residue was purified by silica gel column chromatography (petroleum ether/ethyl acetate=1/0 to 15/1) to afford 7-nitrochromane.

Step 5: To a solution of 7-nitrochromane (130 mg, 726 μmol, 1.00 eq) in ethyl acetate (6.00 mL) was added palladium on carbon (10% weight on C) (13.0 mg) under nitrogen atmosphere. The reaction was stirred at 25° C. for 2 h under hydrogen. The mixture was filtered, and the filtrate was concentrated under reduced pressure to afford chroman-7-amine.

Step 6: To a solution of chroman-7-amine (110 mg, 737 μmol, 1.00 eq) and pyridine (0.18 mL, 2.21 mmol, 3.00 eq) in acetonitrile (5.00 mL) was added phenyl chloroformate (0.11 mL, 885 μmol, 1.20 eq) at 25° C. The reaction was stirred at 25° C. for 12 h. The mixture was concentrated and the obtained residue was purified by standard methods to afford phenyl chroman-7-ylcarbamate.

Compound 293: General procedure B with variant i) was used for the preparation from compound VIII-B employing phenyl (3-cyclopropoxyphenyl)carbamate. ¹H NMR (400 MHz, DMSO-d₆) δ=11.00 (s, 1H), 9.81 (s, 1H), 7.80 (s, 1H), 7.72-7.62 (m, 2H), 7.28 (s, 1H), 7.21-7.14 (m, 1H), 7.05 (br d, J=8.3 Hz, 1H), 6.76-6.63 (m, 1H), 5.27 (s, 2H), 5.13 (dd, J=5.1, 13.3 Hz, 1H), 4.54-4.40 (m, 1H), 4.40-4.30 (m, 1H), 3.77 (td, J=3.0, 5.9 Hz, 1H), 3.01-2.83 (m, 1H), 2.61 (br dd, J=2.1, 15.7 Hz, 1H), 2.44-2.35 (m, 1H), 2.06-1.97 (m, 1H), 0.80-0.70 (m, 2H), 0.70-0.61 (m, 2H). MS (ESI) m/z 450.1 [M+H]⁺

Step 1: To a mixture of 3-nitrophenol (0.71 mL, 3.59 mmol, 1.00 eq) and bromocyclopropane (0.86 mL, 10.8 mmol, 3.00 eq) in 1-methyl-2-pyrrolidinone (5.00 mL) was added cesium carbonate (2.34 g, 7.19 mmol, 2.00 eq) in portions. The reaction was stirred at 180° C. for 2 h under microwave irradiation. The mixture was concentrated under reduced pressure to give a residue. The residue was purified by silica gel column chromatography (petroleum ether/ethyl acetate=20/1 to 5/1) to afford 1-cyclopropoxy-3-nitrobenzene.

Step 2: To a solution of 1-cyclopropoxy-3-nitrobenzene (240 mg, 1.34 mmol, 1.00 eq) in tetrahydrofuran (5.00 mL) was added Pd/C (10% weight on C) (50.0 mg) under nitrogen. The reaction was stirred at 20° C. for 1 h under hydrogen (15 psi). The mixture was filtered, and the filtrate was concentrated under reduced pressure to afford 3-cyclopropoxyaniline.

Step 3: To a solution of 3-cyclopropoxyaniline (280 mg, 1.88 mmol, 1.00 eq) and pyridine (0.76 mL, 9.38 mmol, 5.00 eq) in acetonitrile (3.00 mL) was added phenyl chloroformate (0.35 mL, 2.82 mmol, 1.50 eq). The reaction was stirred at 20° C. for 12 h. The mixture was concentrated and the obtained residue was purified by standard methods to afford phenyl (3-cyclopropoxyphenyl)carbamate.

Compound 294: General procedure B with variant i) was used for the preparation from compound VIII-B employing phenyl (5-(piperidin-1-yl)pyrazin-2-yl)carbamate. ¹H NMR (400 MHz, DMSO-d₆) δ=11.00 (s, 1H), 10.03 (s, 1H), 8.47 (s, 1H), 8.03 (d, J=1.2 Hz, 1H), 7.80 (s, 1H), 7.69-7.61 (m, 2H), 5.28 (s, 2H), 5.13 (dd, J=5.1, 13.2 Hz, 1H), 4.54-4.44 (m, 1H), 4.39-4.30 (m, 1H), 3.49 (br s, 4H), 2.97-2.89 (m, 1H), 2.63-2.58 (m, 1H), 2.43-2.35 (m, 1H), 2.05-1.97 (m, 1H), 1.61-1.51 (m, 6H). MS (ESI) m/z 479.2 [M+H]⁺

Step 1: To a solution of 5-chloropyrazin-2-amine (1.00 g, 7.72 mmol, 1.00 eq) and di-tert-butyldicarbonate (1.95 mL, 8.49 mmol, 1.10 eq) in tetrahydrofuran (10.0 mL) was added 4-dimethylaminopyridine (94.3 mg, 772 μmol, 0.10 eq). The reaction was stirred at 20° C. for 2 h. The mixture was concentrated under reduced pressure to give a residue. The residue was purified by silica gel column chromatography (petroleum ether/ethyl acetate=10/1 to 5/1) to afford tert-butyl (5-chloropyrazin-2-yl)carbamate.

Step 2: To a solution of tert-butyl (5-chloropyrazin-2-yl)carbamate (800 mg, 3.48 mmol, 1.00 eq) and piperidine (1.72 mL, 17.4 mmol, 5.00 eq) in dimethylformamide (3.00 mL) was added cesium carbonate (2.27 g, 6.97 mmol, 2.00 eq). The reaction was stirred at 180° C. for 2 h under microwave irradiation. The mixture was diluted with water (50.0 mL) and extracted with ethyl acetate (3×30.0 mL). The combined organic layers were washed with brine (20.0 mL), dried over anhydrous sodium sulfate, filtered, and concentrated under reduced pressure to give a residue. The residue was purified by reversed phase preparative HPLC to afford 5-(piperidin-1-yl) pyrazin-2-amine.

Step 3: To a solution of 5-(piperidin-1-yl)pyrazin-2-amine (50.0 mg, 281 μmol, 1.00 eq) and pyridine (0.07 mL, 842 μmol, 3.00 eq) in acetonitrile (1.00 mL) was added phenyl chloroformate (0.05 mL, 421 μmol, 1.50 eq). The reaction was stirred at 20° C. for 3 h. The mixture was concentrated and the obtained residue was purified by standard methods to afford phenyl (5-(piperidin-1-yl)pyrazin-2-yl) carbamate.

Compound 295: General procedure B with variant i) was used for the preparation from compound VIII-B employing phenyl (3-(difluoromethoxy)-5-(morpholinomethyl)phenyl)carbamate. ¹H NMR (400 MHz, DMSO-d₆) δ=11.00 (s, 1H), 10.01 (s, 1H), 8.19 (s, 1H), 7.80 (s, 1H), 7.71-7.62 (m, 2H), 7.29 (s, 2H), 7.17 (d, J=74 Hz, 1H), 6.76 (s, 1H), 5.29 (s, 2H), 5.13 (dd, J=5.1, 13.3 Hz, 1H), 4.52-4.44 (m, 1H), 4.39-4.31 (m, 1H), 3.60-3.55 (m, 4H), 3.42 (s, 2H), 2.97-2.87 (m, 1H), 2.64-2.58 (m, 1H), 2.45-2.40 (m, 1H), 2.35 (br s, 4H), 2.06-1.98 (m, 1H). MS (ESI) m/z 559.2 [M+H]⁺

Step 1: To a solution of 3-hydroxy-5-nitrobenzoic acid (1.80 g, 9.83 mmol, 1.00 eq) in dimethylformamide (10.0 mL) were added morpholine (0.86 mL, 9.83 mmol, 1.00 eq), 0-(7-azabenzotriazol-1-yl)-N,N,N′,N′-tetramethyluronium hexafluorophosphate (7.45 g, 19.6 mmol, 2.00 eq) and diisopropylethylamine (5.16 mL, 29.6 mmol, 3.00 eq). The reaction was stirred at 25° C. for 2 h. The mixture was extracted with ethyl acetate/water (200 ml/100 ml). The organic layer was collected, and the solvents were partly removed under reduced pressure to give a concentrated solution. The solution was purified by reversed phase preparative HPLC to afford (3-hydroxy-5-nitrophenyl)(morpholino)methanone.

Step 2: To a solution of (3-hydroxy-5-nitrophenyl)(morpholino)methanone (1.77 g, 7.02 mmol, 1.00 eq) in dimethylformamide (15.0 mL) were added (2-chloro-2,2-difluoro-acetyl)oxysodium (2.67 g, 17.5 mmol, 2.50 eq) and cesium carbonate (4.57 g, 14.0 mmol, 2.00 eq). The reaction was stirred at 100° C. for 2 h. The mixture was extracted with water/ethyl acetate (100 ml/100 ml). The organic layer was collected, and the solvents were partly removed under reduced pressure to give a concentrated solution. The solution was purified by reversed phase preparative HPLC to afford (3-(difluoromethoxy)-5-nitrophenyl)(morpholino)methanone.

Step 3: To a solution of (3-(difluoromethoxy)-5-nitrophenyl)(morpholino)methanone (1.39 g, 4.60 mmol, 1.00 eq) in tetrahydrofuran (5.00 mL) was added borane dimethyl sulfide complex (2 M in THF) (0.92 mL, 2.00 eq) at 25° C. The mixture was stirred at 25° C. for 0.5 h, then at 60° C. for 1.5 h. Methanol (5.00 mL) was added, and the mixture was extracted with water/ethyl acetate (50.0 ml/50.0 ml). The organic layer was collected, and the solvents were partly removed under reduced pressure to give a concentrated solution. The solution was purified by reversed phase preparative HPLC to afford 4-(3-(difluoromethoxy)-5-nitrobenzyl)morpholine.

Step 4: To a solution of 4-(3-(difluoromethoxy)-5-nitrobenzyl)morpholine (795 mg, 2.76 mmol, 1.00 eq) in methanol (15.0 mL) and water (5.00 mL) were added iron power (770 mg, 13.8 mmol, 5.00 eq) and ammonium chloride (1.18 g, 22.0 mmol, 8.00 eq). The reaction was stirred at 80° C. for 2 h. The mixture was filtered, and the filtrate was concentrated under reduced pressure to give a concentrated solution. The solution was extracted with ethyl acetate/saturated sodium bicarbonate (40.0 ml/10.0 ml). The organic layer was collected, dried over sodium sulfate, filtered, and concentrated under reduced pressure to afford 3-(difluoromethoxy)-5-(morpholinomethyl)aniline.

Step 5: To a solution of 3-(difluoromethoxy)-5-(morpholinomethyl)aniline (100 mg, 387 μmol, 1.00 eq) in acetonitrile (2.00 mL) were added pyridine (0.10 mL, 1.18 mmol, 3.04 eq) and phenyl chloroformate (0.07 mL, 599 μmol, 1.55 eq). The reaction was stirred at 25° C. for 2 h. The mixture was filtered, and the filtrate was purified by standard methods to afford phenyl (3-(difluoromethoxy)-5-(morpholinomethyl)phenyl)carbamate.

Compound 296: General procedure B with variant i) was used for the preparation from compound VIII-B employing phenyl(3-(difluoromethoxy)-5-(2-morpholinoethoxy)phenyl)carbamate. ¹H NMR (400 MHz, DMSO-d₆) δ=10.99 (s, 1H), 9.97 (s, 1H), 7.79 (s, 1H), 7.70-7.66 (m, 1H), 7.65-7.61 (m, 1H), 7.39-6.99 (m, 1H), 6.97-6.89 (m, 2H), 6.43 (t, J=2.1 Hz, 1H), 5.28 (s, 2H), 5.19-5.05 (m, 1H), 4.51-4.42 (m, 1H), 4.39-4.30 (m, 1H), 4.04 (t, J=5.7 Hz, 2H), 3.62-3.52 (m, 4H), 2.98-2.85 (m, 1H), 2.67 (t, J=5.6 Hz, 2H), 2.60 (br d, J=17.7 Hz, 1H), 2.48-2.40 (m, 4H), 2.40-2.31 (m, 1H), 2.06-1.96 (m, 1H). MS (ESI) m/z 589.4 [M+H]⁺

Step 1: A mixture of 3-bromo-5-nitrophenol (5.00 g, 22.9 mmol, 1.00 eq), 4-(2-chloroethyl)morpholine (4.12 g, 27.5 mmol, 1.20 eq) and caesium carbonate (22.4 g, 68.8 mmol, 3.00 eq) in dimethylformamide (50.0 mL) was stirred at 60° C. for 12 h. The mixture was diluted with ethyl acetate (250 mL) and water (150 mL). The organic layer was separated, and the aqueous layer was extracted with ethyl acetate (3×150 mL). The combined organic layers were dried over sodium sulfate, filtered, and concentrated under reduced pressure to give a residue. The residue was purified by column chromatography on silica gel (petroleum ether/ethyl acetate=3/1) to afford 4-(2-(3-bromo-5-nitrophenoxy)ethyl)morpholine.

Step 2: To a solution of 4-(2-(3-bromo-5-nitro-phenoxy)ethyl)morpholine (5.00 g, 15.1 mmol, 1.00 eq) and potassium hydroxide (2.54 g, 45.3 mmol, 3.00 eq) in dioxane (10.0 mL) and water (10.0 mL) were added tris(dibenzylideneacetone)dipalladium(0) (1.38 g, 1.51 mmol, 0.10 eq) and di-tert-butyl-(2-(2,4,6-tri(propan-2-yl)phenyl)phenyl)phosphane (641 mg, 1.51 mmol, 0.10 eq) under nitrogen. The reaction was stirred at 80° C. for 12 h. The mixture was diluted with ethyl acetate (150 mL) and water (150 mL). The organic layer was separated, and the aqueous layer was extracted with ethyl acetate (3×150 mL). The combined organic layers were dried over sodium sulfate, filtered, and concentrated under reduced pressure to give a residue. The residue was purified by reversed phase preparative HPLC to afford 3-(2-morpholinoethoxy)-5-nitrophenol.

Step 3: A mixture of 3-(2-morpholinoethoxy)-5-nitrophenol (300 mg, 1.12 mmol, 1.00 eq), potassium carbonate (309 mg, 2.24 mmol, 2.00 eq) and sodium 2-chloro-2,2-difluoroacetate (682 mg, 4.47 mmol, 4.00 eq) in dimethylformamide (2.00 mL) and water (0.50 mL) was stirred at 100° C. for 12 h. The mixture was diluted with ethyl acetate (100 mL) and water (100 mL). The organic layer was separated, and the aqueous layer was extracted with ethyl acetate (3×80.0 mL). The combined organic layers were dried over sodium sulfate, filtered, and concentrated under reduced pressure to afford 4-(2-(3-(difluoromethoxy)-5-nitrophenoxy)ethyl)morpholine.

Step 4: A mixture of 4-(2-(3-(difluoromethoxy)-5-nitrophenoxy)ethyl)morpholine (0.50 g, 1.57 mmol, 1.00 eq) and palladium on carbon (10% weight on C) (50.0 mg) in methanol (5.00 mL) was stirred at 25° C. under hydrogen for 3 h. The mixture was filtered over Celite, and the filtrate was concentrated under reduced pressure to afford 3-(difluoromethoxy)-5-(2-morpholinoethoxy)aniline.

Step 5: A mixture of 3-(difluoromethoxy)-5-(2-morpholinoethoxy)aniline (130 mg, 451 μmol, 1.00 eq), phenyl chloroformate (68.0 μL, 541 μmol, 1.20 eq) and pyridine (0.11 mL, 1.35 mmol, 3.00 eq) in acetonitrile (2.00 mL) was stirred at 25° C. for 12 h. The mixture was diluted with ethyl acetate (50.0 mL) and water (50.0 mL). The organic layer was separated, and the aqueous layer was extracted with ethyl acetate (3×100 mL). The combined organic layers were dried over sodium sulfate, filtered, and concentrated and the obtained residue was purified by standard methods to afford phenyl (3-(difluoromethoxy)-5-(2-morpholinoethoxy)phenyl)carbamate.

Compound 297: General procedure B with variant ii) was used for the preparation from compound VIII-B employing (2-methoxyphenyl)methanamine. ¹H NMR (400 MHz, DMSO-d₆) δ=10.98 (br s, 1H), 7.73 (s, 1H), 7.70 (br t, J=6.0 Hz, 1H), 7.61 (s, 2H), 7.25-7.13 (m, 2H), 6.98-6.86 (m, 2H), 5.16 (s, 2H), 5.14-5.08 (m, 1H), 4.49-4.42 (m, 1H), 4.36-4.29 (m, 1H), 4.18 (d, J=6.0 Hz, 2H), 3.78 (s, 3H), 2.97-2.85 (m, 1H), 2.60 (td, J=2.0, 15.4 Hz, 1H), 2.40 (br dd, J=4.4, 13.1 Hz, 1H), 2.05-1.96 (m, 1H). MS (ESI) m/z 438.1 [M+H]⁺

Compound 298: General procedure B with variant ii) was used for the from compound VIII-B employing phenylmethanamine. ¹H NMR (400 MHz, DMSO-d₆) δ=10.98 (br d, J=2.6 Hz, 1H), 7.90 (br t, J=6.2 Hz, 1H), 7.73 (s, 1H), 7.61 (s, 1H), 7.46-7.15 (m, 6H), 5.17 (s, 2H), 5.13 (dd, J=5.1, 13.4 Hz, 1H), 4.50-4.43 (m, 1H), 4.36-4.30 (m, 1H), 4.22 (d, J=6.1 Hz, 2H), 2.92 (ddd, J=5.4, 13.5, 17.4 Hz, 1H), 2.63-2.58 (m, 1H), 2.40 (br dd, J=4.4, 13.2 Hz, 1H), 2.05-1.98 (m, 1H). MS (ESI) m/z 408.1 [M+H]⁺

Compound 299: General procedure B with variant i) was used for the preparation from compound VIII-B employing phenyl (4-(1,1-difluoroethyl)phenyl)carbamate. ¹H NMR (400 MHz, DMSO-d₆) δ=11.00 (br s, 1H), 10.04 (s, 1H), 7.81 (s, 1H), 7.72-7.67 (m, 1H), 7.67-7.62 (m, 1H), 7.61-7.53 (m, 2H), 7.53-7.45 (m, 2H), 5.29 (s, 2H), 5.13 (dd, J=5.1, 13.3 Hz, 1H), 4.54-4.43 (m, 1H), 4.39-4.29 (m, 1H), 2.98-2.86 (m, 1H), 2.61 (td, J=2.1, 15.3 Hz, 1H), 2.44-2.35 (m, 1H), 2.06-1.99 (m, 1H), 1.99-1.88 (m, 3H). MS (ESI) m/z 438.1 [M+H]⁺

To a solution of 4-(1,1-difluoroethyl)aniline·HCl (50.0 mg, 258 μmol, 1.00 eq, HCl salt) in acetonitrile (5.00 mL) were added pyridine (0.10 mL, 1.29 mmol, 5.00 eq) and phenyl chloroformate (42.0 μL, 335 μmol, 1.30 eq). The reaction was stirred at 25° C. for 0.5 h. The mixture was concentrated under reduced pressure to give a residue. The residue was purified by preparative silica gel TLC to afford phenyl (4-(1,1-difluoroethyl)phenyl)carbamate.

Compound 300: General procedure B with variant i) was used for the preparation from compound VIII-B employing phenyl (3-(2-oxa-5-azabicyclo[2.2.1]heptan-5-ylmethyl)-5-(difluoromethoxy)phenyl) carbamate. ¹H NMR (400 MHz, DMSO-d₆) δ=11.00 (s, 1H), 9.99 (s, 1H), 8.17 (s, 1H), 7.80 (s, 1H), 7.71-7.62 (m, 2H), 7.32 (s, 1H), 7.26 (s, 1H), 7.13 (d, J=74 Hz, 1H), 6.78 (s, 1H), 5.29 (s, 2H), 5.13 (dd, J=5.1, 13.2 Hz, 1H), 4.46 (s, 1H), 4.38-4.32 (m, 2H), 3.90 (d, J=7.5 Hz, 1H), 3.68 (d, J=4.5 Hz, 2H), 3.53 (dd, J=1.6, 7.4 Hz, 1H), 3.45 (s, 1H), 2.97-2.88 (m, 1H), 2.71 (s, 1H), 2.63 (br s, 1H), 2.41 (br d, J=9.4 Hz, 2H), 2.05-1.99 (m, 1H), 1.80 (br d, J=8.2 Hz, 1H), 1.60 (br d, J=9.5 Hz, 1H). MS (ESI) m/z 571.2 [M+H]⁺

Step 1: To a solution of 3-hydroxy-5-nitrobenzoic acid (2.80 g, 15.3 mmol, 1.00 eq) in dimethylformamide (5.00 mL) were added 2-oxa-5-azabicyclo[2.2.1]heptane·HCl (3.11 g, 22.9 mmol, 1.50 eq, HCl), O-(7-azabenzotriazol-1-yl)-N,N,N′,N′-tetramethyluronium hexafluorophosphate (11.6 g, 30.6 mmol, 2.00 eq) and diisopropylethylamine (8.00 mL, 45.8 mmol, 3.00 eq). The reaction was stirred at 25° C. for 2 h. The mixture was extracted with water/ethyl acetate (100 ml/200 ml). The organic layer was separated, and most of the solvent was removed under reduced pressure to give a concentrated solution. The solution was purified by reversed phase preparative HPLC to afford 2-oxa-5-azabicyclo[2.2.1]heptan-5-yl(3-hydroxyl-5-nitrophenyl)methanone.

Step 2: To a solution of 2-oxa-5-azabicyclo[2.2.1]heptan-5-yl(3-hydroxy-5-nitrophenyl)methanone (3.79 g, 14.3 mmol, 1.00 eq) in dimethylformamide (150 mL) were added (2-chloro-2,2-difluoro-acetyl)oxysodium (5.47 g, 35.8 mmol, 2.50 eq) and cesium carbonate (9.35 g, 28.7 mmol, 2.00 eq). The reaction was stirred at 100° C. for 2 h. The mixture was extracted with water/ethyl acetate (100 ml/100 ml). The organic layer was separated, and most of the solvent was removed under reduced pressure to give a concentrated solution. The solution was purified by reversed phase preparative HPLC to afford 2-oxa-5-azabicyclo[2.2.1]heptan-5-yl(3-(difluoromethoxy)-5-nitrophenyl)methanone.

Step 3: To a solution of 2-oxa-5-azabicyclo[2.2.1]heptan-5-yl(3-(difluoromethoxy)-5-nitrophenyl)methanone (1.82 g, 5.79 mmol, 1.00 eq) in tetrahydrofuran (5.00 mL) was added borane dimethyl sulfide complex (10 M) (1.16 mL, 2.00 eq) at 25° C. under nitrogen. The reaction was stirred at 25° C. for 0.5 h, then at 60° C. for 1.5 h. Methanol (5.00 ml) was added, and the mixture was extracted with water/ethyl acetate (50.0 ml/50.0 ml). The organic layer was separated, and most of the solvent was removed under reduced pressure to give a concentrated solution. The solution was purified by reversed phase preparative HPLC to afford 5-(3-(difluoromethoxy)-5-nitrobenzyl)-2-oxa-5-azabicyclo[2.2.1]heptane.

Step 4: To a solution of 5-(3-(difluoromethoxy)-5-nitrobenzyl)-2-oxa-5-azabicyclo[2.2.1]heptane (902 mg, 3.00 mmol, 1.00 eq) in methanol (15.0 mL) and water (5.00 mL) were added iron power (839 mg, 15.0 mmol, 5.00 eq) and ammonium chloride (1.29 g, 24.0 mmol, 8.00 eq). The reaction was stirred at 80° C. for 2 h. The mixture was filtered, and the filtrate was concentrated under reduced pressure to give a concentrated solution. The solution was extracted with ethyl acetate/saturated sodium bicarbonate (40.0 mL/10.0 mL). The organic layer was collected, dried over sodium sulfate, filtered, and concentrated under reduced pressure to afford 3-(2-oxa-5-azabicyclo[2.2.1]heptan-5-ylmethyl)-5-(difluoromethoxy)aniline.

Step 5: To a solution of 3-(2-oxa-5-azabicyclo[2.2.1]heptan-5-ylmethyl)-5-(difluoromethoxy)aniline (200 mg, 740 μmol, 1.00 eq) in acetonitrile (2.00 mL) were added pyridine (0.18 mL, 2.23 mmol, 3.00 eq) and phenyl chloroformate (0.14 mL, 1.12 mmol, 1.50 eq). The reaction was stirred at 25° C. for 2 h. The mixture was filtered, and the filtrate was purified by standard methods to afford phenyl (3-(2-oxa-5-azabicyclo[2.2.1]heptan-5-ylmethyl)-5-(difluoromethoxy)phenyl)carbamate.

Compound 301: General procedure B with variant i) was used for the preparation from compound VIII-B employing phenyl (4-(2,2-difluorocyclopropyl)phenyl)carbamate. ¹H NMR (400 MHz, DMSO-d₆) δ=10.99 (s, 1H), 9.82 (br s, 1H), 7.79 (s, 1H), 7.71-7.58 (m, 2H), 7.44 (br d, J=8.6 Hz, 2H), 7.18 (d, J=8.6 Hz, 2H), 5.27 (s, 2H), 5.13 (dd, J=5.0, 13.2 Hz, 1H), 4.52-4.42 (m, 1H), 4.39-4.30 (m, 1H), 2.96-2.86 (m, 2H), 2.62 (br s, 1H), 2.46-2.36 (m, 1H), 2.05-1.97 (m, 1H), 1.96-1.81 (m, 2H). MS (ESI) m/z 470.1 [M+H]⁺

Step 1: To a solution of 1-nitro-4-vinylbenzene (500 mg, 3.35 mmol, 1.00 eq) and sodium iodide (251 mg, 1.68 mmol, 0.50 eq) in 1,2-dimethoxyethane (5.00 mL) was added (trifluoromethyl)trimethylsilane (1.19 g, 8.38 mmol, 2.50 eq) dropwise under nitrogen. The reaction was stirred at 150° C. for 2 h under microwave irradiation. The mixture was concentrated under reduced pressure to give a residue. The residue was purified by silica gel column chromatography (petroleum ether/ethyl acetate=10/1) to afford 1-(2,2-difluorocyclopropyl)-4-nitrobenzene.

Step 2: To a solution of 1-(2,2-difluorocyclopropyl)-4-nitrobenzene (380 mg, 1.91 mmol, 1.00 eq) in ethanol (8.00 mL) and water (2.00 mL) were added iron power (533 mg, 9.54 mmol, 5.00 eq) and ammonium chloride (510 mg, 9.54 mmol, 5.00 eq) in one portion. The reaction was stirred at 80° C. for 1 h. The mixture was diluted with water (80.0 mL) and extracted with ethyl acetate (3×60.0 mL). The combined organic layers were washed with brine (30.0 mL), dried over sodium sulfate, filtered, and concentrated under reduced pressure to afford 4-(2,2-difluorocyclopropyl)aniline.

Step 3: To a solution of 4-(2,2-difluorocyclopropyl)aniline (300 mg, 1.77 mmol, 1.00 eq) and pyridine (0.72 mL, 8.87 mmol, 5.00 eq) in acetonitrile (5.00 mL) was added phenyl chloroformate (0.27 mL, 2.13 mmol, 1.20 eq) dropwise at 0° C. The reaction was stirred at 25° C. for 12 h. The mixture was concentrated and the obtained residue was purified by standard methods to afford phenyl (4-(2,2-difluorocyclopropyl) phenyl)carbamate.

Compound 302: General procedure B with variant ii) was used for the preparation from compound VIII-B employing phenylmethanamine. ¹H NMR (400 MHz, DMSO-d₆) δ=10.98 (br s, 1H), 7.89 (br d, J=8.2 Hz, 1H), 7.72 (s, 1H), 7.59 (s, 2H), 7.30 (d, J=4.4 Hz, 4H), 7.23-7.18 (m, 1H), 5.15-5.07 (m, 3H), 4.67 (quin, J=7.4 Hz, 1H), 4.48-4.41 (m, 1H), 4.35-4.28 (m, 1H), 2.96-2.86 (m, 1H), 2.63-2.57 (m, 1H), 2.39 (br dd, J=4.4, 13.2 Hz, 1H), 2.04-1.97 (m, 1H), 1.34 (d, J=7.0 Hz, 3H). MS (ESI) m/z 422.2 [M+H]⁺

Compound 303: General procedure B with variant i) was used for the preparation from compound VIII-B employing phenyl (3-methyl-2,3-dihydrobenzofuran-6-yl)carbamate. ¹H NMR (400 MHz, DMSO-d₆) δ=11.10-10.94 (m, 1H), 9.81-9.65 (m, 1H), 8.45 (s, 1H), 7.79 (s, 1H), 7.70-7.61 (m, 2H), 7.15-7.03 (m, 1H), 6.99-6.83 (m, 1H), 5.25 (s, 2H), 5.13 (dd, J=5.1, 13.2 Hz, 1H), 4.64 (t, J=8.8 Hz, 1H), 4.53-4.44 (m, 1H), 4.40-4.29 (m, 1H), 4.00 (dd, J=7.5, 8.5 Hz, 1H), 3.50-3.46 (m, 1H), 2.95-2.87 (m, 1H), 2.62 (br d, J=2.5 Hz, 1H), 2.41-2.31 (m, 1H), 2.04-1.98 (m, 1H), 1.22 (d, J=6.9 Hz, 3H). MS (ESI) m/z 450.1 [M+H]⁺

Step 1: To a solution of 2-bromo-5-nitro-phenol (1.00 g, 4.59 mmol, 1.00 eq) and potassium carbonate (1.27 g, 9.17 mmol, 2.00 eq) in acetone (10.0 mL) was added 3-bromoprop-1-ene (665 mg, 5.50 mmol, 1.20 eq) at 25° C. The reaction was stirred at 25° C. for 12 h. The mixture was concentrated under reduced pressure to give a residue. Water (80.0 mL) was added, and the mixture was stirred for 10 min. The aqueous layer was extracted with ethyl acetate (3×40.0 mL). The combined organic layers were washed with brine (100 mL), dried over anhydrous sodium sulfate, filtered, and concentrated under reduced pressure to give a residue. The residue was purified by silica gel column chromatography (petroleum ether/ethyl acetate=50/1) to afford 2-(allyloxy)-1-bromo-4-nitrobenzene.

Step 2: To a solution of 2-(allyloxy)-1-bromo-4-nitrobenzene (800 mg, 3.10 mmol, 1.00 eq), sodium acetate (635 mg, 7.75 mmol, 2.50 eq), tetraethylammonium iodide (877 mg, 3.41 mmol, 1.10 eq) and sodium formate (210 mg, 3.10 mmol, 1.00 eq) in dimethylformamide (2.00 mL) was added palladium acetate (139 mg, 620 μmol, 0.20 eq) at 25° C. The reaction was stirred at 100° C. for 12 h. The mixture was poured into water (80.0 mL) and stirred for 10 min. The aqueous layer was extracted with ethyl acetate (3×30.0 mL). The combined organic layers were washed with brine (50.0 mL), dried over anhydrous sodium sulfate, filtered, and concentrated under reduced pressure to give a residue. The residue was purified by silica gel column chromatography (petroleum ether/ethyl acetate=20/1) to afford 3-methyl-6-nitrobenzofuran.

Step 3: To a solution of 3-methyl-6-nitro-benzofuran (150 mg, 846 μmol, 1.00 eq) in methanol (2.00 mL) was added palladium on carbon (10% weight on C) (30.0 mg). The reaction was stirred at 25° C. for 12 h under hydrogen. The mixture was filtered, and the filtrate was concentrated under reduced pressure to afford 3-methyl-2,3-dihydrobenzofuran-6-amine.

Step 4: To a solution of 3-methyl-2,3-dihydrobenzofuran-6-amine (120 mg, 804 μmol, 1.00 eq) and pyridine (0.32 mL, 4.02 mmol, 5.00 eq) in acetonitrile (3.00 mL) was added phenyl chloroformate (0.15 mL, 1.21 mmol, 1.50 eq). The reaction was stirred at 25° C. for 12 h. The mixture was concentrated and the obtained residue was purified by standard methods to afford phenyl (3-methyl-2,3-dihydrobenzofuran-6-yl)carbamate.

Compound 304: General procedure B with variant i) was used for the preparation from compound VIII-B employing phenyl N-(4-cyclobutylphenyl)carbamate. ¹H NMR (400 MHz, DMSO-d₆) δ=11.00 (s, 1H), 9.72 (br s, 1H), 7.79 (s, 1H), 7.72-7.59 (m, 2H), 7.39 (br d, J=8.4 Hz, 2H), 7.15 (d, J=8.4 Hz, 2H), 5.26 (s, 2H), 5.13 (dd, J=5.2, 13.2 Hz, 1H), 4.55-4.43 (m, 1H), 4.40-4.26 (m, 1H), 3.47-3.44 (m, 1H), 2.97-2.83 (m, 1H), 2.63 (br s, 1H), 2.45-2.35 (m, 1H), 2.30-2.21 (m, 2H), 2.09-1.98 (m, 3H), 1.97-1.87 (m, 1H), 1.84-1.73 (m, 1H). MS (ESI) m/z 448.1 [M+H]⁺

Step 1: To a solution of 4-bromoaniline (10.0 g, 58.1 mmol, 1.00 eq) and triethylamine (24.0 mL, 172 mmol, 2.97 eq) in dichloromethane (70.0 mL) was added trifluoroacetic anhydride (12.1 mL, 87.2 mmol, 1.50 eq) dropwise at 0° C. The reaction was stirred at 25° C. for 12 h. The mixture was purified by silica gel column chromatography (petroleum ether/ethyl acetate=10/1) to afford N-(4-bromophenyl)-2,2,2-trifluoro-acetamide.

Step 2: To a solution of N-(4-bromophenyl)-2,2,2-trifluoro-acetamide (2.00 g, 7.46 mmol, 1.00 eq) in tetrahydrofuran (15.0 mL) was added n-Butyllithium (2.50 M, 6.27 mL, 2.10 eq) dropwise at −78° C. The reaction was stirred at −78° C. for 0.5 h. Then cyclobutanone (0.67 mL, 8.95 mmol, 1.20 eq) was added, and the reaction was stirred at −78° C. for 2.5 h. The reaction was quenched by addition of saturated aqueous ammonium chloride, and the mixture was extracted with ethyl acetate (2×20.0 mL). The combined organic layers were washed with brine (20.0 mL), dried over anhydrous sodium sulfate, filtered, and concentrated under reduced pressure to give a residue. The residue was purified by silica gel column chromatography (petroleum ether/ethyl acetate=10/1 to 2/1) to afford 2,2,2-trifluoro-N-(4-(1-hydroxycyclobutyl)phenyl)acetamide.

Step 3: To a solution of 2,2,2-trifluoro-N-(4-(1-hydroxycyclobutyl)phenyl)acetamide (1.60 g, 6.17 mmol, 1.00 eq) in methanol (10.0 mL) was added sodium hydroxide solution (1.00 M, 6.17 mL, 1.00 eq). The reaction was stirred at 25° C. for 12 h. The mixture was concentrated under reduced pressure to give a residue. Water (10.0 mL) was added, and the mixture was extracted with ethyl acetate (2×20.0 mL). The combined organic layers were washed with brine (20.0 mL), dried over anhydrous sodium sulfate, filtered, and concentrated under reduced pressure to give a residue. The residue was purified by silica gel column chromatography (petroleum ether/ethyl acetate=5/1 to 2/1) to afford 1-(4-aminophenyl)cyclobutanol.

Step 4: To a solution of 1-(4-aminophenyl)cyclobutanol (0.70 g, 4.29 mmol, 1.00 eq) in tetrahydrofuran (10.0 mL) were added sodium borohydride (923 mg, 24.4 mmol, 5.69 eq) and aluminium trichloride (1.72 g, 12.9 mmol, 3.00 eq) in portions. The reaction was stirred at 70° C. for 3 h. The mixture was poured into water (100 mL) in portions. The aqueous layer was extracted with ethyl acetate (2×20.0 mL). The combined organic layers were washed with brine (30.0 mL), dried over anhydrous sodium sulfate, filtered, and concentrated under reduced pressure to give a residue. The residue was purified by silica gel column chromatography (petroleum ether/ethyl acetate=10/1 to 2/1) to afford 4-cyclobutylaniline.

Step 5: To a solution of 4-cyclobutylaniline (200 mg, 1.36 mmol, 1.00 eq) and pyridine (0.55 mL, 6.79 mmol, 5.00 eq) in acetonitrile (3.00 mL) was added phenyl chloroformate (0.20 mL, 1.63 mmol, 1.20 eq). The reaction was stirred at 25° C. for 1 h. The mixture was concentrated and the obtained residue was purified by standard methods to afford phenyl N-(4-cyclobutylphenyl)carbamate.

Compound 305: General procedure B with variant i) was used for the preparation from compound VIII-B employing phenyl (6-cyclobutylpyridin-3-yl)carbamate. ¹H NMR (400 MHz, DMSO-d₆) δ=10.99 (s, 1H), 10.81 (s, 1H), 8.80 (d, J=2.1 Hz, 1H), 8.37 (dd, J=2.3, 8.8 Hz, 1H), 7.95 (d, J=8.8 Hz, 1H), 7.81 (s, 1H), 7.73-7.68 (m, 1H), 7.67-7.61 (m, 1H), 5.35 (s, 2H), 5.11 (dd, J=5.1, 13.3 Hz, 1H), 4.52-4.45 (m, 1H), 4.38-4.31 (m, 1H), 3.92 (br d, J=8.8 Hz, 1H), 2.96-2.87 (m, 1H), 2.60 (br d, J=17.5 Hz, 1H), 2.43-2.32 (m, 5H), 2.10-1.98 (m, 2H), 1.88 (dtd, J=3.2, 7.9, 11.1 Hz, 1H). MS (ESI) m/z 449.2 [M+H]⁺

Step 1: To a solution of zinc bromide (2.49 g, 11.0 mmol, 3.50 eq) in tetrahydrofuran (15.0 mL) was added cyclobutylmagnesium bromide (0.5 M, 17.6 mL, 2.80 eq) dropwise at −78° C. under nitrogen. The reaction was stirred at −78° C. for 0.5 h, then at 0° C. for 0.5 h. 2-chloro-5-nitropyridine (500 mg, 3.15 mmol, 1.00 eq) and tetrakis(triphenylphosphine)palladium(0) (364 mg, 315 μmol, 0.10 eq) were added successively. The reaction was stirred at 0° C. for 10 min. Then the reaction was warmed to 60° C. and stirred at 60° C. for 2 h. The mixture was diluted with saturated ammonium chloride (100 mL) and extracted with ethyl acetate (3×80.0 mL). The combined organic layers were washed with brine (60.0 mL), dried over sodium sulfate, filtered, and concentrated under reduced pressure to give a residue. The residue was purified by silica gel column chromatography (petroleum ether/ethyl acetate=5/1) to afford 2-cyclobutyl-5-nitro-pyridine.

Step 2: To a solution of 2-cyclobutyl-5-nitropyridine (320 mg, 1.80 mmol, 1.00 eq) in methanol (10.0 mL) was added palladium on carbon (10% weight on C) (50.0 mg) in one portion. The reaction was stirred at 25° C. for 2 h under hydrogen (15 Psi). The mixture was filtered, and the filtrate was concentrated under reduced pressure to afford 6-cyclobutylpyridin-3-amine.

Step 3: To a solution of 6-cyclobutylpyridin-3-amine (230 mg, 1.55 mmol, 1.00 eq) and pyridine (0.63 mL, 7.76 mmol, 5.00 eq) in acetonitrile (5.00 mL) was added phenyl chloroformate (0.23 mL, 1.86 mmol, 1.20 eq) dropwise at 0° C. The reaction was stirred at 25° C. for 12 h. The mixture was concentrated under reduced pressure to give a residue. Dimethylformamide (2.00 mL) was added, and the solution was filtered. The filtrate was concentrated by standard methods to afford phenyl (6-cyclobutylpyridin-3-yl)carbamate.

Compound 306: General procedure B with variant i) was used for the preparation from compound VIII-B employing phenyl (3-(2-morpholinoethoxy)-5-(trifluoromethoxy)phenyl)carbamate. ¹H NMR (400 MHz, DMSO-d₆) δ=11.09-10.90 (m, 1H), 10.08 (s, 1H), 8.23 (br s, 1H), 7.79 (s, 1H), 7.67 (br d, J=1.1 Hz, 1H), 7.66-7.62 (m, 1H), 7.14 (s, 1H), 7.07 (s, 1H), 6.59 (s, 1H), 5.28 (s, 2H), 5.17-5.04 (m, 1H), 4.51-4.43 (m, 1H), 4.38-4.30 (m, 1H), 4.06 (t, J=5.7 Hz, 2H), 3.60-3.53 (m, 4H), 2.97-2.86 (m, 1H), 2.67 (t, J=5.6 Hz, 2H), 2.64-2.57 (m, 1H), 2.48-2.42 (m, 4H), 2.40-2.31 (m, 1H), 2.05-1.97 (m, 1H). MS (ESI) m/z 607.4 [M+H]⁺

Step 1: To a solution of 3-(2-morpholinoethoxy)-5-nitrophenol (110 mg, 410 μmol, 1.00 eq) in dimethylformamide (3.00 mL) was added sodium hydride (60% dispersion in mineral oil) (32.8 mg, 820 μmol, 2.00 eq) in portions at 0° C. The reaction was stirred at 0° C. for 0.5 h, then dibromodifluoromethane (75.8 μL, 820 μmol, 2.00 eq) was added dropwise. The reaction was stirred at 25° C. for 1 h. Saturated aqueous ammonium chloride solution (20.0 mL) was added, and the mixture was extracted with ethyl acetate (2×20.0 mL). The combined organic layers were washed with brine (10.0 mL), dried over sodium sulfate, filtered, and concentrated under reduced pressure to give a residue. The residue was purified by preparative silica gel TLC (petroleum ether/ethyl acetate=1/1) to afford 4-(2-(3-(bromodifluoromethoxy)-5-nitrophenoxy)ethyl)morpholine.

Step 2: To a solution of 4-(2-(3-(bromodifluoromethoxy)-5-nitrophenoxy)ethyl)morpholine (90.0 mg, 227 μmol, 1.00 eq) in dichloromethane (3.00 mL) was added silver tetrafluoroborate (88.2 mg, 453 μmol, 2.00 eq) in portions. The reaction was stirred at 25° C. for 1 h. The mixture was filtered, and the filtrate was concentrated under reduced pressure to afford 4-(2-(3-nitro-5-(trifluoromethoxy)phenoxy)ethyl)morpholine.

Step 3: A mixture of 4-(2-(3-nitro-5-(trifluoromethoxy)phenoxy)ethyl)morpholine (300 mg, 892 μmol, 1.00 eq) and palladium on carbon (10% weight on C) (100 mg) in methanol (10.0 mL) was stirred at 25° C. under hydrogen for 3 h. The mixture was filtered over Celite, and the filtrate was concentrated under reduced pressure to afford 3-(2-morpholinoethoxy)-5-(trifluoromethoxy)aniline.

Step 4: A solution of 3-(2-morpholinoethoxy)-5-(trifluoromethoxy)aniline (110 mg, 359 μmol, 1.00 eq), phenyl chloroformate (67.5 μL, 539 μmol, 1.50 eq) and pyridine (87.0 μL, 1.08 mmol, 3.00 eq) in acetonitrile (10.0 mL) was stirred at 25° C. for 12 h. The mixture was diluted with ethyl acetate (30.0 mL) and water (30.0 mL). The organic layer was separated, and the aqueous phase was extracted with ethyl acetate (3×30.0 mL). The combined organic layers were dried over sodium sulfate, filtered, and concentrated and the obtained residue was purified by standard methods to afford phenyl (3-(2-morpholinoethoxy)-5-(trifluoromethoxy) phenyl)carbamate.

Compound 307: General procedure B with variant i) was used for the preparation from compound VIII-B employing phenyl (6-(azetidin-1-yl)pyridin-3-yl)carbamate. ¹H NMR (400 MHz, DMSO-d₆) δ=10.99 (s, 1H), 10.12-9.91 (m, 1H), 8.09 (br s, 1H), 7.84 (br d, J=7.8 Hz, 1H), 7.78 (s, 1H), 7.71-7.60 (m, 2H), 6.80 (br d, J=7.9 Hz, 1H), 5.28 (s, 2H), 5.12 (dd, J=5.1, 13.4 Hz, 1H), 4.51-4.41 (m, 1H), 4.38-4.29 (m, 1H), 4.27-4.06 (m, 4H), 2.96-2.87 (m, 1H), 2.63 (br s, 1H), 2.47-2.33 (m, 3H), 2.05-1.98 (m, 1H). MS (ESI) m/z 450.1 [M+H]⁺

Step 1: To a solution of azetidine·HCl (5.14 mL, 46.5 mmol, 1.50 eq, HCl) and 2-fluoro-5-nitropyridine (4.40 g, 30.9 mmol, 1.00 eq) in dimethylformamide (40.0 mL) was added potassium carbonate (12.8 g, 92.9 mmol, 3.00 eq) in one portion. The reaction was stirred at 60° C. for 12 h. The mixture was poured into water (60.0 mL) and filtered. The filtrate was concentrated under reduced pressure to afford 2-(azetidin-1-yl)-5-nitropyridine.

Step 2: To a solution of 2-(azetidin-1-yl)-5-nitropyridine (2.00 g, 11.2 mmol, 1.00 eq) in methanol (40.0 mL) was added palladium on carbon (10% weight on C) (300 mg). The reaction was stirred at 25° C. for 2 h under hydrogen (15 Psi). The mixture was filtered, and the filtrate was concentrated under reduced pressure to afford 6-(azetidin-1-yl)pyridin-3-amine.

Step 3: To a solution of 6-(azetidin-1-yl)pyridin-3-amine (700 mg, 4.69 mmol, 1.00 eq) and pyridine (1.89 mL, 23.5 mmol, 5.00 eq) in acetonitrile (10.0 mL) was added phenyl chloroformate (0.71 mL, 5.63 mmol, 1.20 eq) dropwise at 0° C. The reaction was stirred at 25° C. for 12 h. The mixture was concentrated and the obtained residue was purified by standard methods to afford phenyl (6-(azetidin-1-yl)pyridin-3-yl)carbamate.

### Example 2: IC50 Data—Compound Activity by Fluorescent Polarization Assay

Compound activity was monitored in a fluorescence polarization (FP) homogeneous assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Unless otherwise stated, all reagents were purchased from Sigma Aldrich. Enzymatic reactions were conducted in Perkin-Elmer Black 384 well ProxiPlate Plus (catalogue no. 6008269) in 10 μL total volume. Full length wild-type cereblon CRBN (80.0 nM, 10 μL) was incubated in assay buffer containing 20 mM HEPES (pH 8.0), 150 NaCl, 0.5 mM TCEP and 0.05% Tween 20 in the presence or absence of compound (300 nL). Inhibitors were stored as 10 mM DMSO stocks in an inert environment (low humidity, dark, low oxygen, room temperature) using the Storage Pod System. Compounds and DMSO were dispensed using the Echo E5XX (Labcyte Inc. USA) to give concentrations from 300 to 0.937 or 3000 to 9.3 nM in a 12 data point curve. Mutant YWAA CRBN (80.0 nM, 10 μL) which does not interact with the fluorescent probe was used as a negative control for the assay. Following incubation at room temperature for 30 min, the assay was initiated by dispensing the probe to a final concentration of 5 nM (2.5 nL of a 20 μM stock) using the Echo E5XX. FP was measured after a period of 12 hours using a Pherastar plate reader (BMG Labtech, Germany) exciting at 590 nm and measuring the amount of parallel and perpendicular light at 675 nm. The FP signal was subsequently normalized to the no-compound control (i.e., DMSO). Analysis and IC50 values were derived using Dotmatics (Dotmatics UK) software.

TABLE 3
IC50 values of compounds 1 to 165 and 201 to 307 determined in the
fluorescence polarization assay indicating the cereblon binding

| | rFP | | rFP | | rFP |
|---|---|---|---|---|---|
| | IC50 | | IC50 | | IC50 |
| Compound | [nM] | Compound | [nM] | Compound | [nM] |
| 1 | B | 2 | A | 3 | A |
| 4 | A | 5 | A | 6 | A |
| 7 | A | 8 | A | 9 | B |
| 10 | B | 11 | C | 12 | B |
| 13 | B | 14 | C | 15 | B |
| 16 | B | 17 | B | 18 | B |
| 19 | B | 20 | B | 21 | B |
| 22 | A | 23 | A | 24 | C |
| 25 | B | 26 | B | 27 | A |
| 28 | B | 29 | C | 30 | B |
| 31 | B | 32 | B | 33 | B |
| 34 | A | 35 | B | 36 | B |
| 37 | B | 38 | B | 39 | B |
| 40 | B | 41 | B | 42 | B |
| 43 | B | 44 | B | 45 | A |
| 46 | C | 47 | B | 48 | B |
| 49 | B | 50 | A | 51 | A |
| 52 | A | 53 | A | 54 | A |
| 55 | A | 56 | A | 57 | A |
| 58 | B | 59 | A | 60 | A |
| 61 | A | 62 | A | 63 | A |
| 64 | A | 65 | B | 66 | A |
| 67 | A | 68 | A | 69 | A |
| 70 | A | 71 | A | 72 | A |
| 73 | A | 74 | A | 75 | A |
| 76 | A | 77 | B | 78 | B |
| 79 | A | 80 | B | 81 | B |
| 82 | A | 83 | A | 84 | A |
| 85 | A | 86 | A | 87 | A |
| 88 | A | 89 | A | 90 | A |
| 91 | A | 92 | A | 93 | A |
| 94 | A | 95 | A | 96 | A |
| 97 | A | 98 | A | 99 | A |
| 100 | A | 101 | A | 102 | A |
| 103 | A | 104 | A | 105 | A |
| 106 | A | 107 | A | 108 | B |
| 109 | A | 110 | A | 111 | A |
| 112 | A | 113 | A | 114 | A |
| 115 | A | 116 | A | 117 | A |
| 118 | A | 119 | A | 120 | A |
| 121 | A | 122 | B | 123 | B |
| 124 | A | 125 | B | 126 | A |
| 127 | A | 128 | A | 129 | A |
| 130 | A | 131 | A | 132 | A |
| 133 | A | 134 | A | 135 | A |
| 136 | A | 137 | C | 138 | B |
| 139 | B | 140 | B | 141 | A |
| 142 | B | 143 | D | 144 | B |
| 145 | B | 146 | A | 147 | A |
| 148 | A | 149 | C | 150 | A |
| 151 | B | 152 | B | 153 | D |
| 154 | B | 155 | B | 156 | B |
| 157 | C | 158 | C | 159 | B |
| 160 | A | 161 | A | 162 | B |
| 163 | C | 164 | C | 165 | C |
| 201 | C | 202 | B | 203 | C |
| 204 | B | 205 | C | 206 | B |
| 207 | B | 208 | B | 209 | C |
| 210 | C | 211 | D | 212 | C |
| 213 | B | 214 | C | 215 | B |
| 216 | A | 217 | D | 218 | B |
| 219 | D | 220 | C | 221 | D |
| 222 | D | 223 | A | 224 | B |
| 225 | B | 226 | B | 227 | D |
| 228 | C | 229 | B | 230 | A |
| 231 | D | 232 | D | 233 | B |
| 234 | D | 235 | A | 236 | A |
| 237 | D | 238 | B | 239 | B |
| 240 | A | 241 | B | 242 | D |
| 243 | D | 244 | B | 245 | D |
| 246 | A | 247 | C | 248 | C |
| 249 | C | 250 | D | 251 | B |
| 252 | B | 253 | B | 254 | A |
| 255 | B | 256 | C | 257 | D |
| 258 | B | 259 | A | 260 | A |
| 261 | D | 262 | B | 263 | A |
| 264 | B | 265 | A | 266 | A |
| 267 | B | 268 | B | 269 | A |
| 270 | A | 271 | C | 272 | B |
| 273 | A | 274 | A | 275 | A |
| 276 | B | 277 | B | 278 | A |
| 279 | C | 280 | A | 281 | A |
| 282 | A | 283 | B | 284 | A |
| 285 | D | 286 | B | 287 | B |
| 288 | B | 289 | B | 290 | C |
| 291 | B | 292 | B | 293 | B |
| 294 | A | 295 | C | 296 | C |
| 297 | B | 298 | B | 299 | C |
| 300 | B | 301 | D | 302 | B |
| 303 | B | 304 | D | 305 | B |
| 306 | B | 307 | A | | |

In some embodiments, the disclosure is directed to compounds with an IC50 value of less than 1100 nM, i.e. directed to compounds 1, 2, 3, 4, 5, 6, 7, 8, 9, 10, 12, 13, 15, 16, 17, 18, 19, 20, 21, 22, 23, 26, 26, 27, 28, 30, 31, 32, 33, 34, 35, 36, 37, 38, 39, 40, 41, 42, 43, 44, 45, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 138, 139, 140, 141, 142, 144, 145, 146, 147, 148, 150, 151, 152, 154, 155, 156, 158, 159, 160, 161,162, 202, 204, 206, 207, 208, 213, 215, 216, 218, 223, 224, 225, 226, 229, 230, 233, 235, 236, 238, 239, 240, 241, 244, 246, 251, 252, 253, 254, 255, 258, 259, 260, 262, 263, 264, 265, 266, 267, 268, 269, 270, 272, 273, 274, 275, 276, 277, 278, 280, 281, 282, 283, 284, 286, 287, 288, 289, 291, 292, 293, 294, 297, 298, 300, 302, 303, 305, 306, and 307.

### Example 3: Compound Binding by Immunofluorescence Assay

In order to demonstrate the ability of the compounds to bind to degrade a specific protein of interest, GSPT1 was chosen and tested in an immunofluorescence assay. CAL-51 cells were purchased from DSMZ (cat. Number ACC302), sub-cultured in 90% Dulbecco's MEM (4.5 g/L glucose, Gibco 11965)+10% heat inactivated FBS (BioConcept, 2-01F1361) and incubated at 37° C., 5% CO₂. For the assay, imaging microtiterplate Cell Carrier 96 Ultra (Perkin Elmer 6055302) were pre-coated with Fibronectin (Sigma F085, 30 μl at 0.2 μg/ml) in PBS (100p, Gibco 14190) for 45 min at room temperature, rinsed with PBS and CAL-51 cells (30K cells/well) were plated and let to adhere overnight. Cells were treated with compounds typically using a serial dilution ranging from 30 μM to 0.1 nM for 6 hours. Compounds were stored at 10 mM DMSO stocks. Vehicle (DMSO), positive (CC-885, 10 μM) and rescue controls (positive control plus 0.2 μM bortezomib) were also included at this stage. Cells were subsequently rinsed with PBS and fixed in 10% Formalin solution (50p, Sigma HT5011)) for 20 mins at room temperature. Following three consecutive PBS washes (100 μl), cells were permeabilized in 0.1% Triton X-100 in PBS (Sigma 93443, 50 μl) for 15 mins at room temperature. Following three further PBS washes, 50 μl blocking buffer (1% BSA, Sigma A4503, in PBS) was added for 45 min for signal-to-noise reduction. Primary antibody (human GSPT1, Sigma HPA052488) was diluted in blocking buffer (dil.1/300, 35 μl/well) and incubated with the cells overnight at 4° C. After three PBS washes, Alexa-fluor 488 coupled secondary antibodies (Invitrogen, A32731, dil. 1/1000), Alexa-fluor 647-Phalloïdin (Invitrogen, A22287, dil. 1/200) and DAPI (Thermo, #62248, dil.1/1000) were diluted in blocking buffer and incubated with the samples for 2 hours at room temperature. After three final PBS washes, samples were conserved in 100 μl PBS in the dark, until measurement. Image acquisition was performed on the Operetta High-Content Imager (Perkin-Elmer). Fluorescence intensity of Alexa-Fluor 488 (GSPT1), Alexa-Fluor 647 (Actin) and DAPI (Nucleus) were measured. For the determination of GSPT1 DC₅₀ values, a custom algorithm implemented in the PerkinElmer image analysis software Harmony-Acapella@ was developed. After user-defined setting of adjustment parameters, the analysis was run identically without human intervention for all image fields. DAPI staining of the nuclei was used to determine the location of cells using standard nuclei detection modules. Segmentation artifacts were removed by threshold-based filters for area, roundness and intensity. The outline of the cells was determined analogously from the sum of the normalized, smoothed DAPI and Actin channel, starting from each nucleus.

The Alexa-Fluor 488 (GSPT1) signal intensity in each cell was finally measured, in order to obtain a Mean intensity per cell. GSPT1 degradation (DC₅₀) was calculated after normalization to controls and data import in CDD vault Database, using non-linear regression.

Table 4 assigns each compound a code indicating the ability for GSPT1 degradation: A, B or C. According to the code, A represents a DC50 value of ≤100 nM, B represents a DC50 value >100 nM and ≤300 nM and C represents a DC50 value of >300 nM.

TABLE 4
Activity for GSPT1 degradation for compounds 1 to 165

| Compound | Code | Compound | Code | Compound | Code |
|---|---|---|---|---|---|
| 1 | C | 2 | C | 3 | C |
| 4 | C | 5 | C | 6 | C |
| 7 | C | 8 | A | 9 | C |
| 10 | C | 11 | C | 12 | C |
| 13 | C | 14 | C | 15 | C |
| 16 | C | 17 | C | 18 | C |
| 19 | C | 20 | A | 21 | C |
| 22 | C | 23 | C | 24 | C |
| 25 | C | 26 | C | 27 | C |
| 28 | C | 29 | C | 30 | A |
| 31 | B | 32 | C | 33 | A |
| 34 | C | 35 | C | 36 | C |
| 37 | C | 38 | B | 39 | C |
| 40 | C | 41 | B | 42 | B |
| 43 | C | 44 | C | 45 | C |
| 46 | A | 47 | A | 48 | C |
| 49 | C | 50 | C | 51 | C |
| 52 | C | 53 | C | 54 | C |
| 55 | C | 56 | A | 57 | C |
| 58 | C | 59 | B | 60 | C |
| 61 | A | 62 | C | 63 | C |
| 64 | C | 65 | C | 66 | C |
| 67 | C | 68 | B | 69 | C |
| 70 | C | 71 | A | 72 | C |
| 73 | C | 74 | C | 75 | B |
| 76 | A | 77 | A | 78 | A |
| 79 | C | 80 | C | 81 | C |
| 82 | A | 83 | C | 84 | C |
| 85 | B | 86 | C | 87 | C |
| 88 | C | 89 | B | 90 | A |
| 91 | C | 92 | C | 93 | C |
| 94 | C | 95 | C | 96 | C |
| 97 | C | 98 | C | 99 | C |
| 100 | C | 101 | B | 102 | C |
| 103 | C | 104 | C | 105 | C |
| 106 | B | 107 | C | 108 | C |
| 109 | B | 110 | C | 111 | C |
| 112 | C | 113 | C | 114 | C |
| 115 | B | 116 | C | 117 | C |
| 118 | A | 119 | A | 120 | C |
| 121 | B | 122 | A | 123 | C |
| 124 | C | 125 | A | 126 | C |
| 127 | A | 128 | C | 129 | A |
| 130 | C | 131 | C | 132 | A |
| 133 | C | 134 | C | 135 | C |
| 136 | C | 137 | C | 138 | A |
| 139 | A | 140 | A | 141 | A |
| 142 | A | 143 | B | 144 | A |
| 145 | A | 146 | C | 147 | C |
| 148 | C | 149 | C | 150 | C |
| 151 | A | 152 | C | 153 | C |
| 154 | C | 155 | B | 156 | C |
| 157 | C | 158 | C | 159 | C |
| 160 | A | 161 | C | 162 | C |
| 163 | A | 164 | C | 165 | C |

Table 5 assigns each compound a code indicating the ability for GSPT1 degradation: A, B or C. According to the code, A represents a DC50 value of ≤30 nM, B represents a DC50 value >30 nM and ≤300 nM and C represents a DC50 value of >300 nM.

TABLE 5
Activity for GSPT1 degradation for compounds 201 to 307

| Compound | Code | Compound | Code | Compound | Code |
|---|---|---|---|---|---|
| 201 | A | 202 | A | 203 | l |
| 204 | A | 205 | A | 206 | A |
| 207 | B | 208 | A | 209 | B |
| 210 | A | 211 | A | 212 | B |
| 213 | A | 214 | C | 215 | A |
| 216 | C | 217 | C | 218 | B |
| 219 | B | 220 | C | 221 | A |
| 222 | C | 223 | C | 224 | A |
| 225 | A | 226 | C | 227 | C |
| 228 | C | 229 | C | 230 | C |
| 231 | B | 232 | B | 233 | B |
| 234 | A | 235 | C | 236 | C |
| 237 | B | 238 | C | 239 | A |
| 240 | B | 241 | A | 242 | A |
| 243 | A | 244 | B | 245 | B |
| 246 | A | 247 | A | 248 | A |
| 249 | A | 250 | A | 251 | B |
| 252 | C | 253 | B | 254 | C |
| 255 | A | 256 | B | 257 | A |
| 258 | A | 259 | B | 260 | B |
| 261 | A | 262 | B | 263 | C |
| 264 | B | 265 | C | 266 | B |
| 267 | B | 268 | A | 269 | A |
| 270 | B | 271 | B | 272 | B |
| 273 | B | 274 | A | 275 | B |
| 276 | B | 277 | A | 278 | B |
| 279 | A | 280 | B | 281 | B |
| 282 | C | 283 | A | 284 | A |
| 285 | A | 286 | A | 287 | A |
| 288 | A | 289 | C | 290 | A |
| 291 | A | 292 | A | 293 | B |
| 294 | B | 295 | A | 296 | A |
| 297 | C | 298 | C | 299 | B |
| 300 | B | 301 | B | 302 | C |
| 303 | B | 304 | B | 305 | B |
| 306 | B | 307 | C | | |

### Example 4: GSPT1 Degradation in Cancer Cell Lines Harboring the R213X Nonsense Mutation

HDQ-P1, ESS-1 and DMS-114 cancer cell lines harboring the R213X nonsense mutation were used to evaluate GSPT1 degradation by a representative compound of the invention, CC-885 and CC-90009. HDQ-P1, ESS-1 cell lines were purchased from the German Collection of Microorganisms and Cell Cultures (DSMZ) and DMS-114 from the ATCC. Cells were treated with the indicated compound for 24 hr, subsequently lysed and GSPT1 levels were measured by WES.

### Example 5: TP53 Readthrough Assay in Cancer Cell Lines Harboring the R213X Nonsense Mutation

Readthrough of the common R213X TP53 nonsense mutation was investigated in the HDQ-P1, ESS-1 and DMS-114 cancer cell lines. HDQ-P1, ESS-1 cell lines were purchased from the German Collection of Microorganisms and Cell Cultures (DSMZ) and DMS-114 from the ATCC. Cells were cultured as recommended by the provider and incubated at 37° C., 5% CO₂. For the readthrough experiment, cells were seeded at 1-2×10⁵ cells per well in six-well plates. The next day, the medium was replaced with fresh medium containing compounds to be tested (concentration range as indicated on the figures) and cells were incubated for 72 hr. The experiments were performed in the presence or absence of Geneticin (G418; typically used at 20 μg/mL). The medium was removed by aspiration, and cells were rinsed with 1 ml ice-cold PBS. Cells were lysed in lysis buffer (20 mM Tris-HCl pH 7.5, 150 mM NaCl, 1 mM EDTA, 1 mM EGTA, 1% (v/v) Triton X100, 2.5 mM sodium pyrophosphate, 1 mM lycerophosphate) supplemented with fresh 1 mM Na3VO4, 1 mM dithiothreitol and 1× complete protease inhibitor cocktail (Roche Molecular Biochemicals). For the western analysis assays, mixtures of cell lysates (0.1-1 mg/mL) and the fluorescent master mix were heated at 95° C. (p53) or 37° C. (GSPT1) for 5 min. The samples, blocking and chemiluminescent reagents, primary (DO-1 mice anti-p53 antibody (1:400; Santa Cruz sc-126), rabbit anti-GSPT1 (1:100, Thermo Fischer Scientific PA5-62621), mouse anti-vinculin antibody (1:600, R&D Systems MAB6896), secondary antibodies, and wash buffer were dispensed into the microplates and capillary electrophoresis western analysis was carried out with the ProteinSimple WES instrument. The data was acquired and analyzed with inbuilt Compass software (ProteinSimple) with the high dynamic range detection profile, which uses multiple substrates injections and exposure times. Electropherograms were converted to pseudo-blots and presented in figures for each visualization.

### Example 6: CFTR Readthrough Reporter Assay

Readthrough of the common G542X cystic fibrosis transmembrane conductance regulator (CFTR) nonsense mutation was carried out in the immortalized human bronchial epithelial cells HBE16. Briefly, a HaloTag G542X 5×5 NanoLuc reporter assay was established in that cell line containing the CFTR readthrough cassette bearing the G542X codon as well as 5 codons upstream and downstream from the human CFTR sequence. The cassette was inserted between the HaloTag and Nanoluc sequences. The construct was transiently transfected into the 16HBE cells and grown in the presence of the compounds in monotherapy or combination with G418 and ELX-02. For these experiments, the concentrations of the GSPT1 degraders used was typically spanning the DC₃₀ to DC₇₀ range (concentrations leading to the degradation of 30 to 70% GSPT1 after 24 hr). G418 and ELX-02 were used at EC10 (concentrations resulting in 10% killing activity after 72 hr; 15 and 85 μM respectively). These concentrations were set so that no significant effect on the HBE16 cell viability was observed during the timecourse of our studies. Readthrough was monitored by measuring the NanoLuc activity (fold-increase) following 48 hr treatment with the compounds alone or in combination with G418 and ELX-02. Graphs were generated using Prism (GraphPad).

1. A method of preventing or treating a disease or disorder caused by or associated with a premature termination codon, comprising administering to a subject a therapeutically effective amount of a compound of formula I 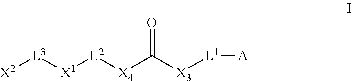
or pharmaceutically acceptable salts or stereoisomer thereof,
wherein A is 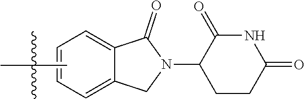
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆ alkyl, —OC(O)—C₁₋₄alkylamino, —C(O)O—C₁₋₆alkyl, —COOH, —CHO, —C₁₋₆alkylC(O)OH, —C₁₋₆ alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen.

2. The method of claim 1, wherein the compound, or pharmaceutically acceptable salt or stereoisomer thereof, is further comprising administering an aminoglycoside or pharmaceutically acceptable salts thereof.

3. The method of claim 1, wherein the compound of formula I, or pharmaceutically acceptable salt or stereoisomer thereof, is a compound of formula IVa, IVb, Va, Vb, VIa, VIb, VIIa or VIIb, 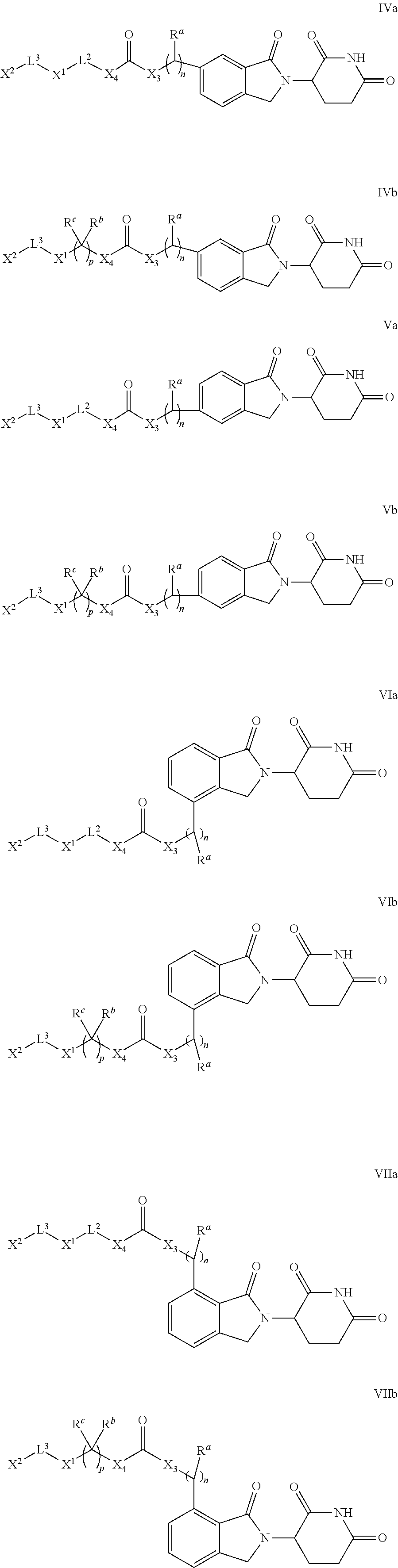
wherein
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆ alkyl, —OC(O)—C₁₋₄alkylamino, —C(O)O—C₁₋₆alkyl, —COOH, —CHO, —C₁₋₆alkylC(O)OH, —C₁₋₆ alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen;
odiesterase 10 inhibitor for the treatment of tourette syndrome”—Your Ref: NOE-001IL-OR^{a} is a H or C₁₋₄ alkyl;
R^{b}, R^{c} are independently of each other H, C₁₋₄ alkyl, such as methyl, ethyl, or halogen, such as F;
n is 0, 1, 2;
p is 0, 1, 2.

4. The method of claim 1, wherein the compound of formula I, or pharmaceutically acceptable salt or stereoisomer thereof, is a compound of formula IXa, IXb, or IXc 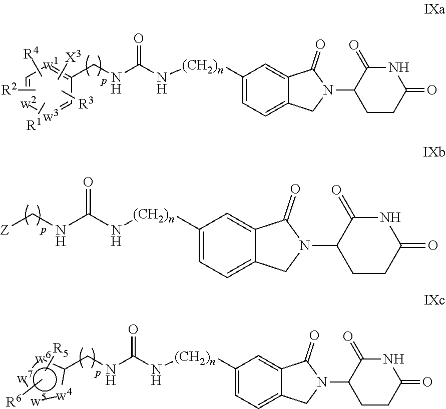
wherein
one of w¹, w² or w³ is selected from C and N, and the other two of w¹, w² or w³ are C;
one or two of w⁴, w⁵, w⁶, w⁷ is selected from C, O, N, NMe, NH, or S while two or three of w⁴, w⁵, w⁶ and w⁷ are C;
X³ is absent, hydrogen or 4-8 membered heterocycloalkyl, C₁₋₄ alkyl 4-8 membered heterocycloalkyl, —O-(4-8 membered heterocycloalkyl), —C₁₋₄ alkoxy-(4-8 membered heterocycloalkyl), 5-10 membered heteroaryl, —O-(5-10 membered heteroaryl), —OC(O)—C₁-4 alkyl-4-8 membered heterocycloalkyl, wherein X³ is unsubstituted or substituted with one or more of linear or branched C₁₋₆ alkyl, NH₂, NMe₂ or 5-6 membered heterocycloalkyl;
Z is linear or branched C₁₋₆ alkyl or C₃₋₆ cycloalkyl, C₁₋₄ alkoxy or C₁₋₄ alkyl-C₁₋₄ alkoxy, wherein Z is unsubstituted or substituted with C₁₋₄ alkyl
R1, R2, R3, R4 each are independently selected from hydrogen, linear or branched C₁₋₆ alkyl, linear or branched C₁₋₆ heteroalkyl, C₆ aryl, such as phenyl, CF₃, CHF₂, CMeF₂, —O—CHF₂—O—(CH₂)₂—OMe, OCF₃, —CN, —N(H)C(O)—C₁₋₆alkyl, —OC(O)—C₁₋₄alkylamino, —OC(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, C₁₋₄ alkylhydroxy, halogen, such as F, Cl, Br, e.g. F or Cl; and/or two of R1, R2, R3, R4 form together a 5-6 membered heterocycloalkyl or a 5-6 membered heteroaryl;
R5, R6 each are independently selected from hydrogen, linear or branched C₁₋₄ alkyl, CF₃, CHF₂, halogen, such as F, Cl, Br, e.g. F or Cl
n is 1 or 2
p is 0, 1 or 2.

5. The method of claim 1, wherein the compound of formula I, or pharmaceutically acceptable salt or stereoisomer thereof, is a compound of formula X, 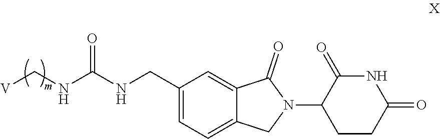
wherein
m is 0, 1, 2 or 3, and
V is selected from

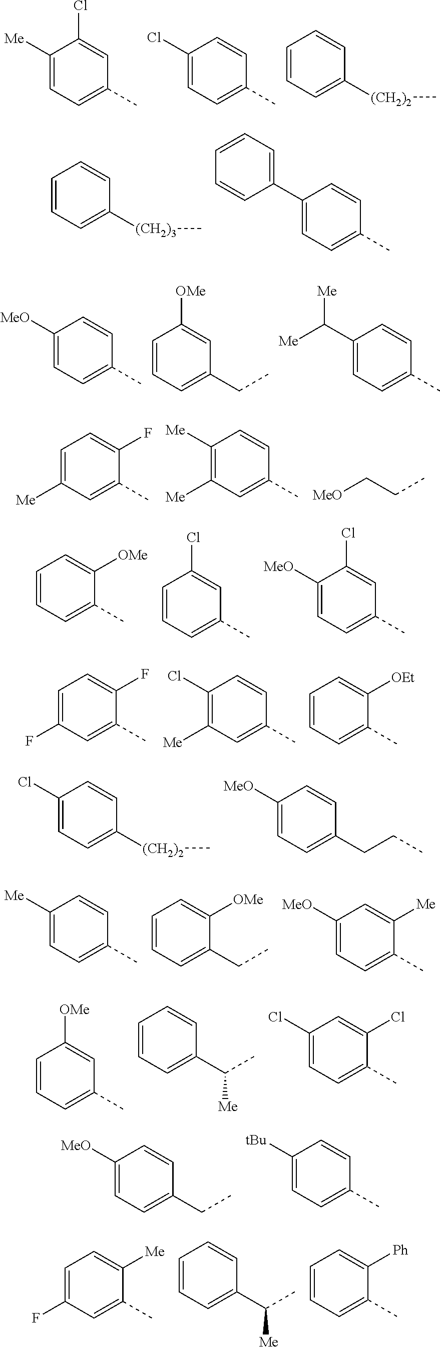

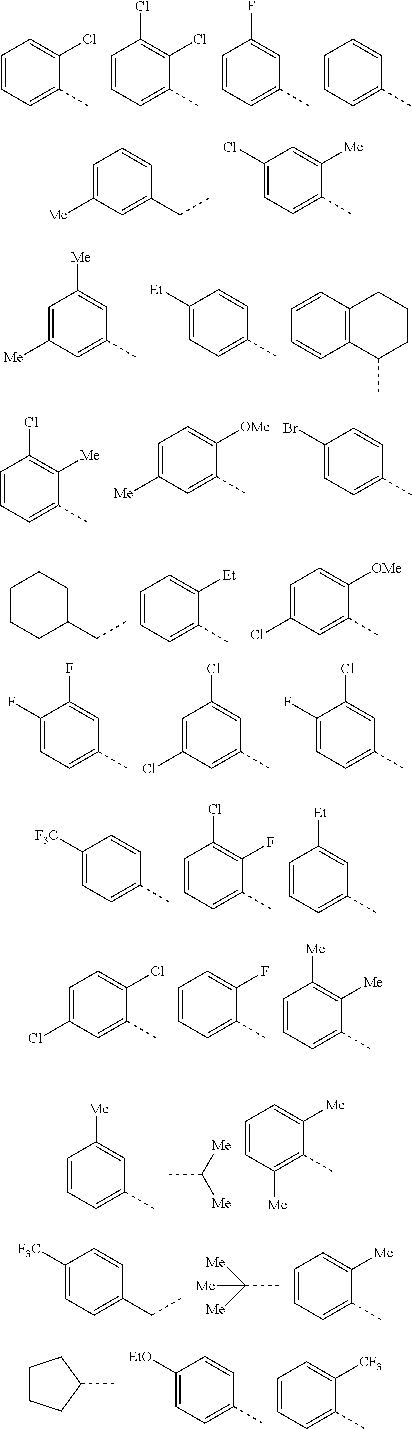

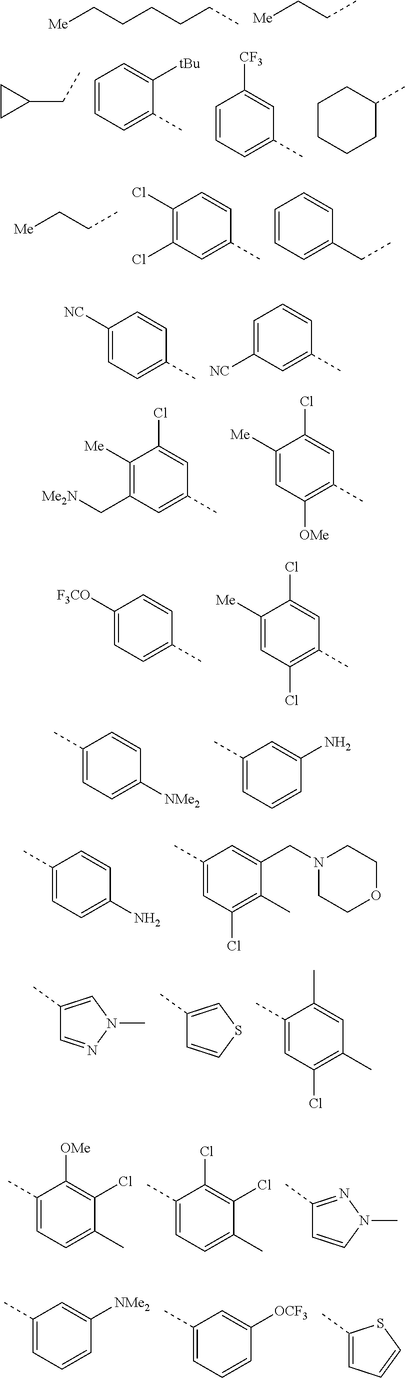

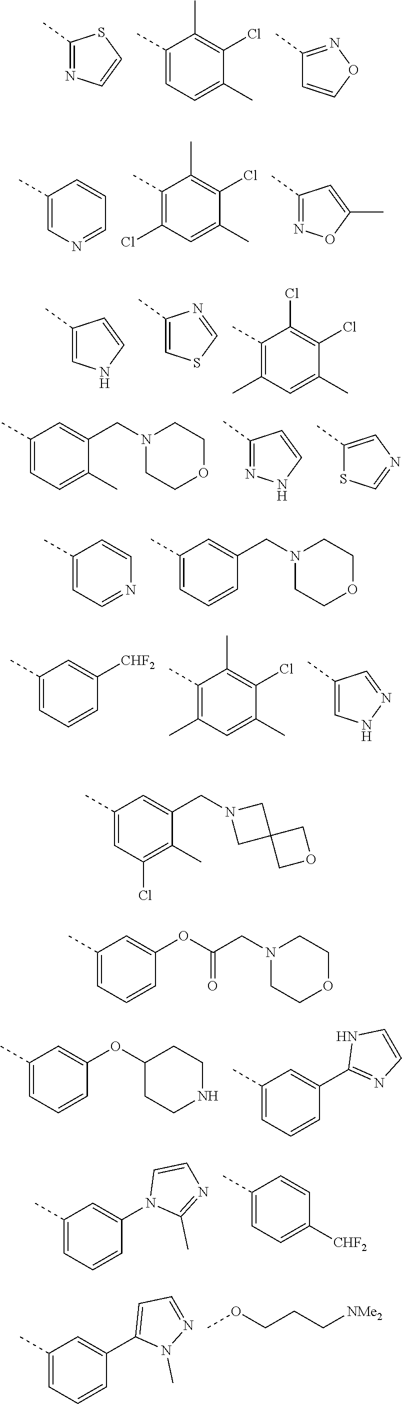

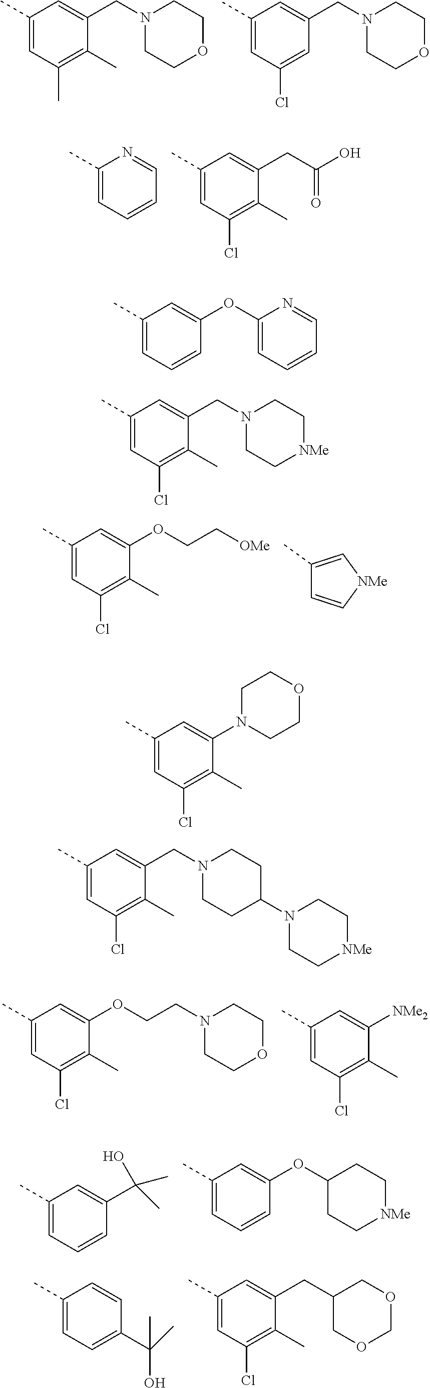

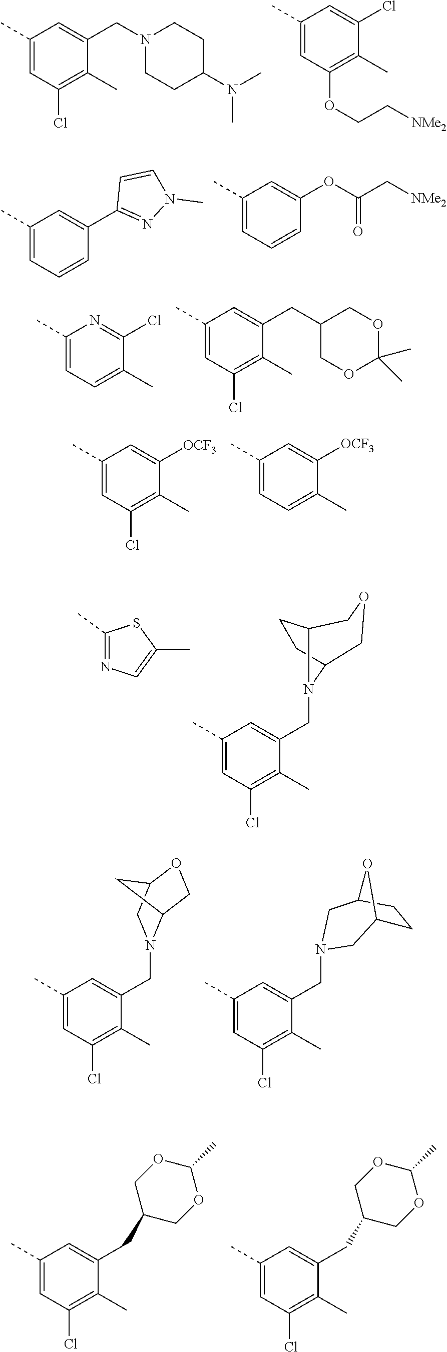

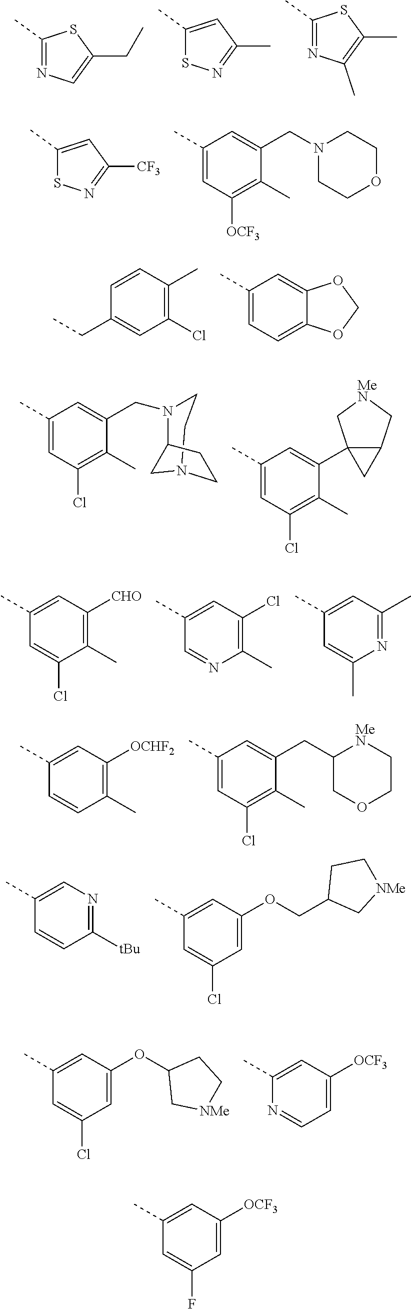

6. The method of claim 1, wherein the compound of formula I, or pharmaceutically acceptable salt or stereoisomer thereof, is a compound of formula of formula XIa, XIb, or XIc 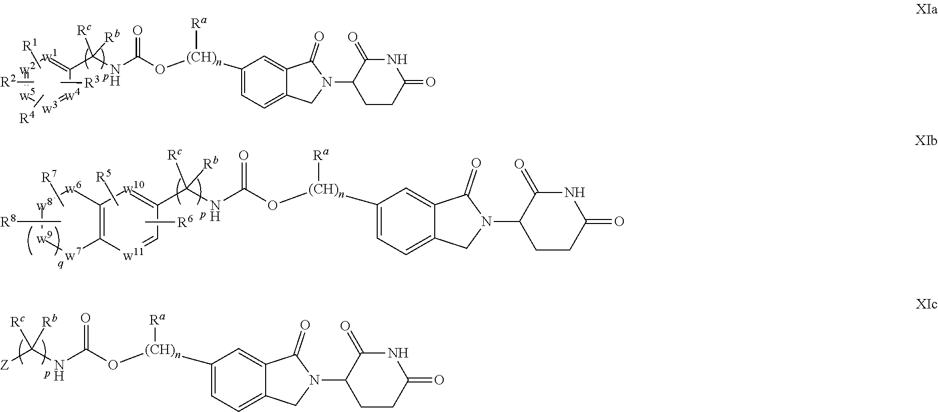
wherein
w¹, w², w³, w⁴, w⁵ are independently of each other selected from C and N, with the proviso that at least three of w¹, w², w³, w⁴, w⁵ are C;
one or two of w⁶, w⁷, w⁸, w⁹ are selected from C and O and the remaining of w⁶, w⁷, w⁸, w⁹ are C;
w¹⁰, w¹¹ are independently of each other selected from C and N;
Z is H, linear or branched —C₁₋₆ alkyl, —C₃₋₆ cycloalkyl, 4-8 membered heterocycloalkyl, wherein Z is unsubstituted or substituted with linear or branched C₁₋₄ alkyl, C₆ aryl, C₆ aryloxy, 6 membered heteroaryl or CF₃; or Z together with the N atom of the carbamate forms a 4-8 membered heterocycloalkyl, which is unsubstituted or substituted with linear or branched C₁₋₄ alkyl, C₆ aryl, C₆ aryloxy, 6 membered heteroaryl or CF₃;
R1, R2, R3, R4 are independently of each other selected from hydrogen, linear or branched —C₁₋₆ alkyl, linear or branched C₁₋₆ heteroalkyl, —C₁₋₆ alkoxy, CF₃, CHF₂, CMeF₂, —O—(CH₂)₂—OMe, OCF₃, OCHF₂, —C₁₋₆ alkylamino, —CN, —OC(O)—C₁₋₆alkyl, —N(H)C(O)—C₁₋₆alkyl, —C(O)O—C₁₋₆alkyl, —COOH, —CHO, —C₁₋₆alkylC(O)OH, —C₁₋₆alkylC(O)O—C₁₋₆alkyl, NH₂, —C₁₋₆ alkylhydroxy, and halogen, such as F, Cl or Br, e.g. F or Cl, or a group of formula -L³-X², wherein L³ is a covalent bond, linear or branched C₁₋₆ alkyl, —O—, —C₁₋₄ alkoxy and X² is C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, and —C₁₋₄ alkylhydroxy;
R5, R6, R7 R8 are independently of each other selected from H, linear or branched C₁₋₄ alkyl, halogen, such as F or Cl, e.g. F;
R^{a} is H, linear or branched C₁₋₄ alkyl,
R^{b}, R^{c} are independently of each other H, linear or branched C₁₋₄ alkyl,
q is 0 or 1,
n is 1 or 2, and
p is 0, 1, or 2.

7. The method of claim 1, wherein the compound of formula I, or pharmaceutically acceptable salt or stereoisomer thereof, is a compound of formula XIIa or XIIb 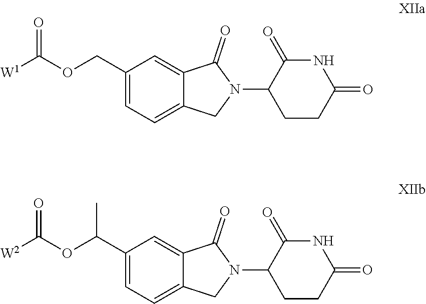
wherein W¹ and W² are selected from

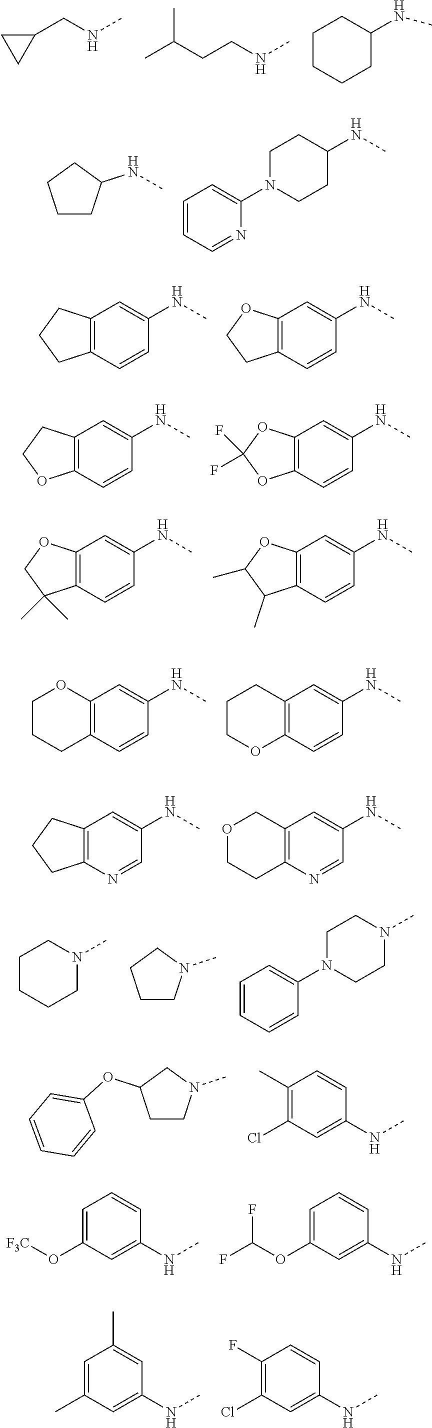

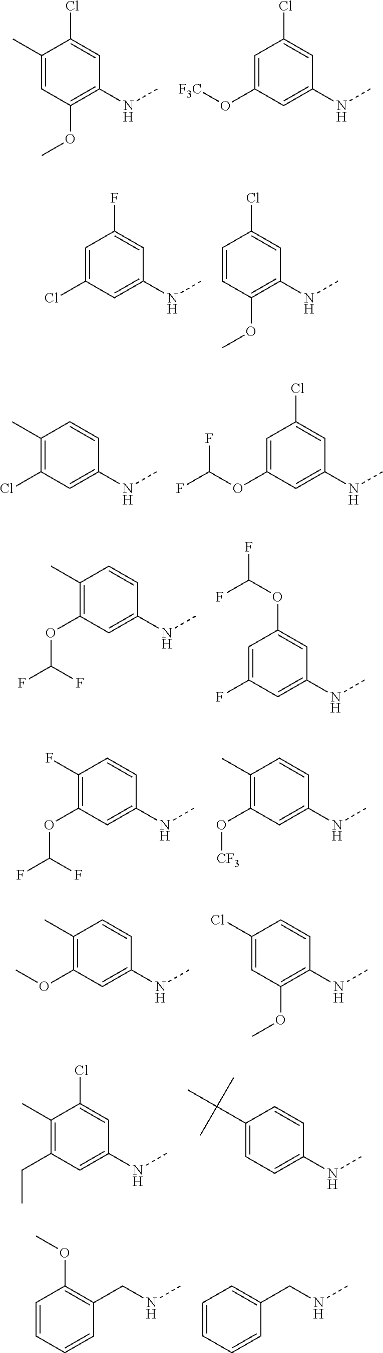

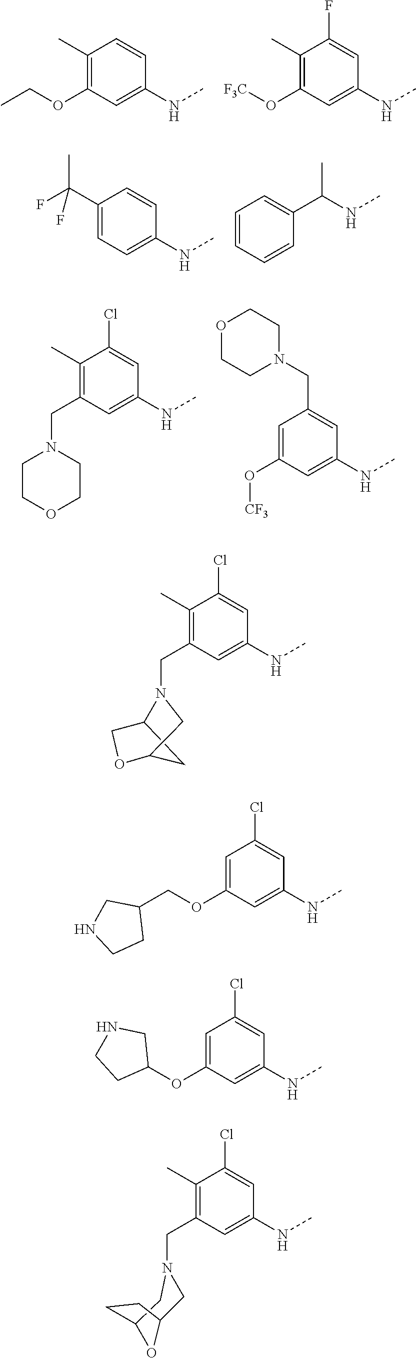

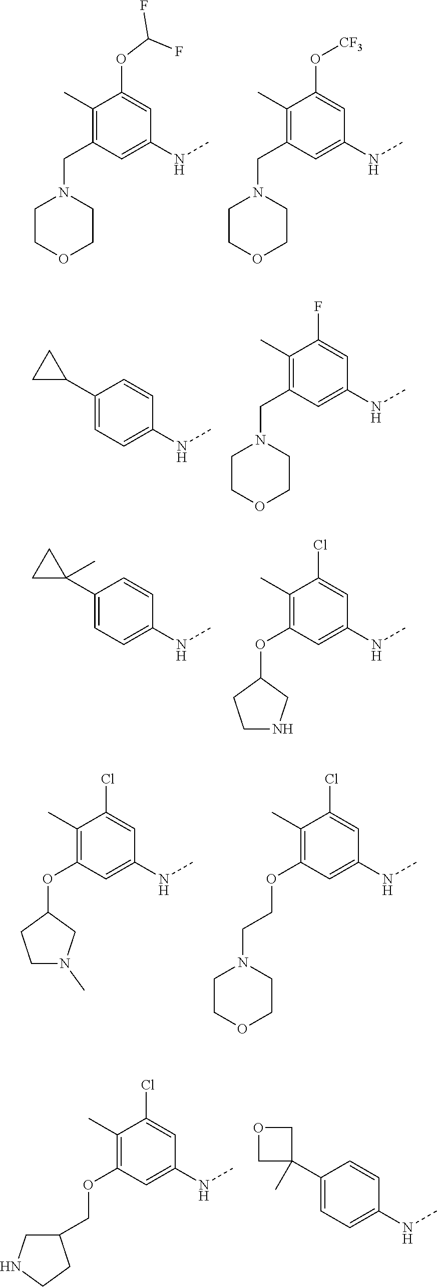

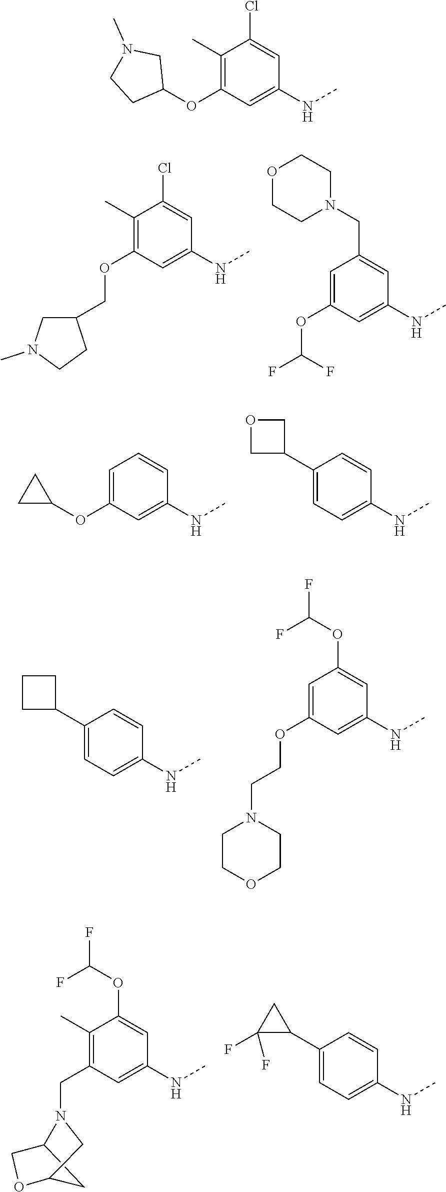

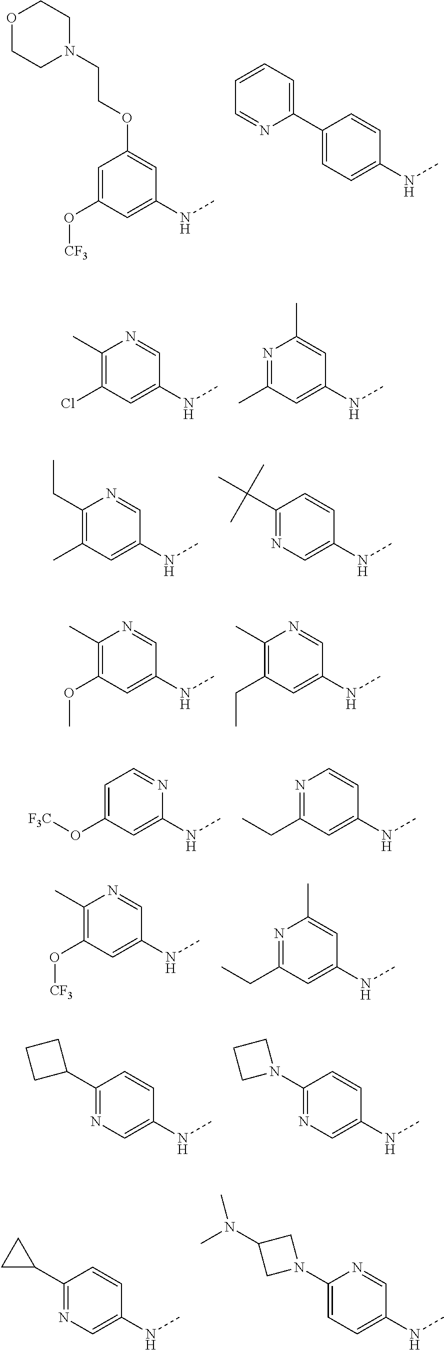

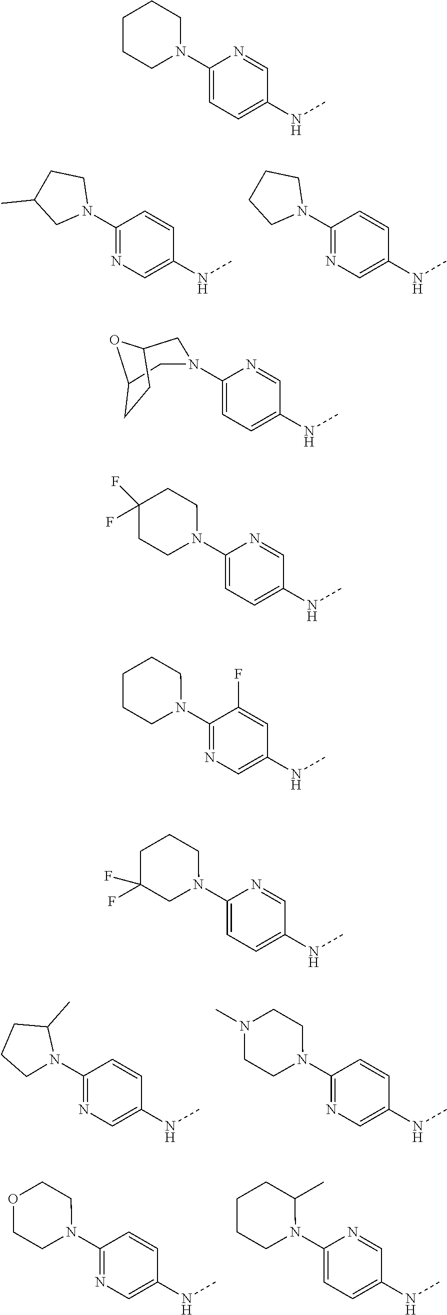

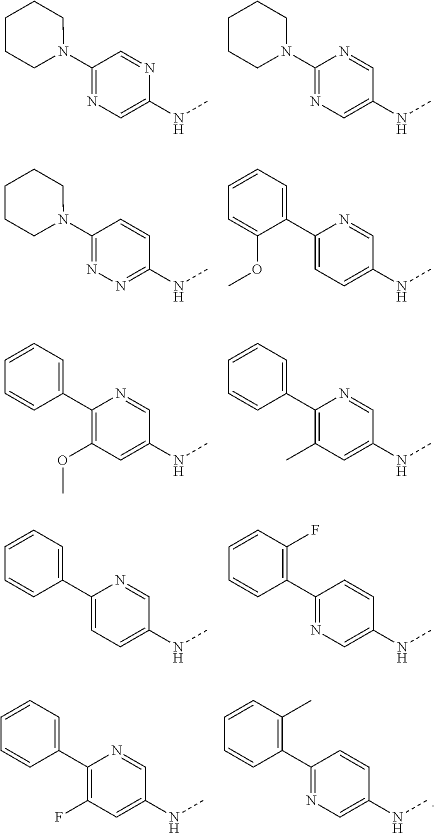

8. The method of claim 2, wherein the aminoglycoside is selected from geneticin, rhodostreptomycin, streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, paromomycin, lividomycin, apramycin, and derivatives thereof.

9. The method of claim 2, wherein the compound of formula I, or pharmaceutically acceptable salt or stereoisomer thereof, and the aminoglycoside are administered in a simultaneous or sequential manner.

10. The method of claim 2, wherein the compound of formula I, or pharmaceutically acceptable salt or stereoisomer thereof, and the aminoglycoside are in form of one single pharmaceutical composition or in form of separate pharmaceutical compositions.

11. The method of claim 1, wherein the premature termination codon is UGA or UAG or UAA.

12. The method of claim 1, wherein the disease or disorder is selected from the group consisting of beta-thalassemia, Ehlers-Danlos syndrome, severe myoclonic epilepsy of infancy, achromatopsia, retinitis pigmentosa, Usher Syndrome Type 1C, adducted thumb-clubfoot syndrome, Alagille syndrome, Alstroem syndrome, antithrombin deficiency, Carney complex, Currarino syndrome, Diamond-Blackfan anemia, erythropoietic protoporphyria, Fabry disease, factor XIII deficiency, Fanconi-Bickel syndrome, fish odor syndrome, Gaucher disease, hereditary hemorrhagic telangiectasia, homocystinuria, Joubert syndrome and related disorders, Krabbe disease, L-2-hydroxyglutaric aciduria, methylmalonic academia, Niemann-Pick disease, Peters plus syndrome, Townes-Brocks disease, von Willebrand disease, Wiskott-Aldrich syndrome, Kabuki syndrome, Chanarin-Dorfman syndrome, lecithin:cholesterol acyltransferase deficiency/fish-eye disease, Marfan Syndrome, mucopolysaccharidiosis, amyloidiosis, Late Infantile Neuronal Ceroid Lipofuscinosis, coenzyme Q10 Deficiency, peroxisome biogenesis disorders, lysosomal storage disorders, colorectal cancer, congenital enteropeptidase deficiency, cystic fibrosis, Hungarian Peutz-Jeghers Syndrome, Jervell and Lange-Nielsen syndrome, Lynch syndrome, microvillus inclusion disease, Peutz-Jeghers syndrome, xanthinuria, acidosis, Alport syndrome, Bardet-Biedl syndrome, Birt-Hogg-Dube syndrome, Dent's disease, Gitelman syndrome, hereditary leiomyomatosis and renal cell cancer, hereditary spherocytosis, leber congenital amaurosis, lysinuric protein intolerance, nephronophthisis, polycystic kidney disease, pseudohypoaldosteronism, renal hypodysplasia, sporadic clear cell renal cell carcinoma, type 2 papillary renal cell cancers, urofacial syndrome, von Hippel-Lindau disease, Wilms' tumor, X-linked Alport syndrome, X-linked hypophosphatemic rickets, hyperuricaemic nephropathy (juvenile/medullary cystic kidney disease), tuberous sclerosis, nephrotic syndrome/congenital nephrotic syndrome, Finnish type nephrotic syndrome, steroid resistant nephrotic syndrome 3, early onset nephrotic syndrome/Pierson syndrome, Denys-Drash syndrome, nephrotic syndrome/Schimke immuno-osseous dysplasia, primary glucocorticoid resistance, X-linked hypophosphatemia, primary hyperoxaluria type 1, pseudohypoaldosteronism type 1, proximal renal tubular acidosis, abetalipoproteinemia and homozygous familial hypobetalipoproteinemia, Alpers syndrome, carbamyl phosphate synthetase I deficiency, cholesteryl ester storage disease, citrin deficiency, Dubin-Johnson syndrome, erythropoietic protoporphyria, factor V deficiency, glycogen storage disease, Hemophilia A (factor VIII Deficiency), Hemophilia B (factor IX Deficiency), hepatocellular carcinoma, hepatoerythropoietic porphyria, hereditary spastic paraplegias, hypobetalipoproteinemia, inherited factor XI deficiency, diabetes mellitus (Type 1 and Type 2), microcytic anemia and iron deficiency, mitochondrial DNA depletion, mitochondrial DNA depletion syndrome, phenylketonuria, polycystic liver disease, porphyria cutanea tarda, progressive familial intrahepatic cholestasis, Wilson Disease, autosomal dominant hypercholesterolemia, factor XII deficiency, factor X deficiency, hypofibrinogenaemia, afibrinogenaemia, factor VII deficiency, agammaglobulinemia, amegakaryocytic thrombocytopenia, dyserythropoietic anemia type II, Duchenne and Becker muscular dystrophy, centronuclear myopathies, limb girdle muscular dystrophy or Miyoshi myopathy, Ullrich disease, spinal muscular atrophy, dystrophic epidermolysis bullosa, Hailey-Hailey Disease, Herlitz junctional epidermolysis bullosa, Netherton syndrome, ataxia-telangiectasia, Dravet syndrome, myotonic dystrophy, infantile neuronal ceroid lipofuscinosis, Alzheimer's disease, Tay-Sachs disease, neural tissue degeneration, Parkinson's disease, lupus erythematosus, graft-versus-host disease, severe combined immunodeficiency, DNA Ligase IV deficiency, Nijmegen breakage disorders, xeroderma pigmentosum (XP), familial erythrocytosis, nephrolithiasis, osteogenesis imperfect, cirrhosis, neurofibroma, bullous disease, lysosomal storage disease, Hurler's disease, familial cholesterolemia; cerebellar ataxia; lung disease; cystic fibrosis; pigmentary retinopathy; amyloidosis, atherosclerosis, gigantism, dwarfism, hypothyroidism, hyperthyroidism, and obesity.

13. The method of claim 1, wherein the disease or disorder is cancer.

14. The method of claim 1, wherein the disease or disorder is cystic fibrosis.

15. The method of claim 1, wherein the disease or disorder is DMD (dystrophin).

16-21. (canceled)

22. A method of promoting readthrough of a premature termination codon of an RNA molecule, comprising providing a compound of formula I or a pharmaceutically acceptable salt or stereoisomer thereof; optionally in combination with an aminoglycoside: 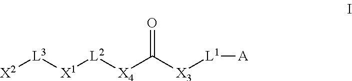
wherein
A is 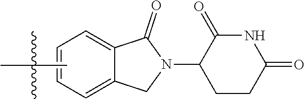
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆ alkyl, —OC(O)—C₁₋₄alkylamino, —C(O)O—C₁₋₆alkyl, —COOH, —CHO, —C₁₋₆alkylC(O)OH, —C₁₋₆ alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen

23. A pharmaceutical combination comprising or consisting of:
(a) a compound of formula I or a pharmaceutically acceptable salt or stereoisomer thereof 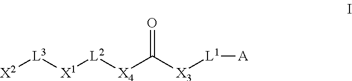
wherein A is 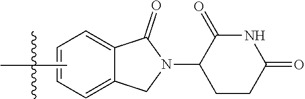
X¹ is linear or branched C₁₋₆ alkyl, C₃₋₆ cycloalkyl, C₆₋₁₀ aryl, 5-10 membered heteroaryl, 4-8 membered heterocycloalkyl, wherein X¹ is unsubstituted or substituted with one or more of halogen, linear or branched C₁₋₆ alkyl, linear or branched C₁₋₆ heteroalkyl, CF₃, CHF₂, CMeF₂, —O—CHF₂, —O—(CH₂)₂—OMe, OCF₃, C₁₋₆ alkylamino, —CN, —N(H)C(O)—C₁₋₆alkyl, —OC(O)—C₁₋₆ alkyl, —OC(O)—C₁₋₄alkylamino, —C(O)O—C₁₋₆alkyl, —COOH, —CHO, —C₁₋₆alkylC(O)OH, —C₁₋₆ alkylC(O)O—C₁₋₆alkyl, NH₂, C₁₋₆ alkoxy or C₁₋₆ alkylhydroxy;
or X¹ together with X⁴ forms a 4-8 membered heterocycloalkyl, which is unsubstituted or substituted with one or more of halogen, linear or branched —C₁₋₆ alkyl, CF₃, CHF₂, CMeF₂, —O—(CH₂)₂—OMe, OCF₃, OCHF₂, C₁₋₆ alkylamino, —CN, —N(H)C(O)—C₁₋₆alkyl, —OC(O)—C₁₋₆alkyl, —C(O)O—C₁₋₆alkyl, —COOH, —C₁₋₆alkylC(O)OH, —C₁₋₆alkylC(O)O—C₁₋₆alkyl, NH₂, C₁₋₄ alkylhydroxy, or C₁₋₆ alkoxy;
X² is hydrogen, C₃₋₆ cycloalkyl, C₆₋₁₀ aryl, 5-10 membered heteroaryl, 4-8 membered heterocycloalkyl, wherein X² is unsubstituted or substituted with one or more of linear or branched C₁₋₆ alkyl, —C₁₋₄ alkoxy, NH₂, NMe₂, halogen, CF₃, CHF₂, CMeF₂, —O—(CH₂)₂—OMe, OCF₃, OCHF₂, C₁₋₄ alkylhydroxy;
X³ is —NH—, —O—;
X⁴ is —NH—, —CH₂—;
L¹ is a covalent bond, C₁₋₆ alkyl, which is unsubstituted or substituted with one or more of C₁₋₄ alkyl, halogen;
L² is a covalent bond, C₁₋₆ alkyl, which is unsubstituted or substituted with one or more of C₁₋₄ alkyl, halogen;
L³ is a covalent bond, —O—, —C₁₋₄ alkoxy or C₁₋₆ alkyl, which is unsubstituted or substituted with one or more of C₁₋₄ alkyl, halogen, and
(b) an aminoglycoside or a pharmaceutically acceptable salt thereof

24. A pharmaceutical combination according to claim 23, wherein the ratio of the compound of formula I to the aminoglycoside is between 100:1 to 1:100 (w/w), such as 50:1 to 1:50 (w/w), e.g. 10:1 to 1:10 (w/w).

25-30. (canceled)

31. The method of claim 1, wherein the compound of formula I, or pharmaceutically acceptable salt or stereoisomer thereof, is a compound of formula IV 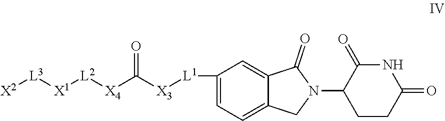
wherein:
L¹ is —CH₂—;
X³ is —O—;
X⁴ is —NH—;
L² is a covalent bond;
X¹ is phenyl substituted with F and OCF₃;
L³ is a covalent bond; and
X² is hydrogen.
